SEQUENCE LISTING (114 sequences)

SEQ ID NO: 1 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
gtggtggacttctctcaat

SEQ ID NO: 2 - RNA, 21 nt, synthetic construct
  source 1..21: mol_type = other RNA
  modified_base order(1,3..5,7..13,15,17..21): mod_base = OTHER; note = 2-prime-O-methyl RNA nucleotides
  modified_base order(2,6,14,16): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
antgagagaagtccaccacga

SEQ ID NO: 3 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
gtggtggacttctctcaat

SEQ ID NO: 4 - RNA, 21 nt, synthetic construct
  source 1..21: mol_type = other RNA
  modified_base order(1,3..5,7..13,15,17..21): mod_base = OTHER; note = 2-prime-O-methyl RNA nucleotides
  modified_base order(2,6,14,16): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
attgagagaagtcnaccacga

SEQ ID NO: 5 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
gtggtggacttctctcaat

SEQ ID NO: 6 - RNA, 21 nt, synthetic construct
  source 1..21: mol_type = other RNA
  modified_base order(1,3..5,7..13,15,17..21): mod_base = OTHER; note = 2-prime-O-methyl RNA nucleotides
  modified_base order(2,6,14,16): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
attgagagaagtccancacga

SEQ ID NO: 7 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
gtggtggacttctctcaat

SEQ ID NO: 8 - RNA, 21 nt, synthetic construct
  source 1..21: mol_type = other RNA
  modified_base order(1,3..5,7..13,15,17..21): mod_base = OTHER; note = 2-prime-O-methyl RNA nucleotides
  modified_base order(2,6,14,16): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
attgagagaagtcnancacga

SEQ ID NO: 9 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
gtggtggacttctctcaat

SEQ ID NO: 10 - RNA, 21 nt, synthetic construct
  source 1..21: mol_type = other RNA
  modified_base order(1,3..5,7..13,15,17..21): mod_base = OTHER; note = 2-prime-O-methyl RNA nucleotides
  modified_base order(2,6,14,16): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
abtgagagaagtccaccacga

SEQ ID NO: 11 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
gtggtggacttctctcaat

SEQ ID NO: 12 - RNA, 21 nt, synthetic construct
  source 1..21: mol_type = other RNA
  modified_base order(1,3..5,7..13,15,17..21): mod_base = OTHER; note = 2-prime-O-methyl RNA nucleotides
  modified_base order(2,6,14,16): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
attgagagaagtcbaccacga

SEQ ID NO: 13 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
gtggtggacttctctcaat

SEQ ID NO: 14 - RNA, 21 nt, synthetic construct
  source 1..21: mol_type = other RNA
  modified_base order(1,3..5,7..13,15,17..21): mod_base = OTHER; note = 2-prime-O-methyl RNA nucleotides
  modified_base order(2,6,14,16): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
attgagagaagtcbabcacga

SEQ ID NO: 15 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
gtggtggacttctctcaat

SEQ ID NO: 16 - RNA, 21 nt, synthetic construct
  source 1..21: mol_type = other RNA
  modified_base order(1,3..5,7..13,15,17..21): mod_base = OTHER; note = 2-prime-O-methyl RNA nucleotides
  modified_base order(2,6,14,16): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
attgagagaagtcnaccacga

SEQ ID NO: 17 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
ccgtgtgcacttcgcttca

SEQ ID NO: 18 - RNA, 21 nt, synthetic construct
  source 1..21: mol_type = other RNA
  modified_base order(1,3,4,6,7,9..13,15,16,18..21): mod_base = OTHER; note = 2-prime-O-methyl RNA nucleotides
  modified_base order(2,5,8,14,17): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
tgaagcgaagtgcacacggtc

SEQ ID NO: 19 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
ccgtgtgcacttcgcttca

SEQ ID NO: 20 - RNA, 21 nt, synthetic construct
  source 1..21: mol_type = other RNA
  modified_base order(1,3,4,6,7,9..13,15,16,18..21): mod_base = OTHER; note = 2-prime-O-methyl RNA nucleotides
  modified_base order(2,5,8,14,17): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
tgaagcgaagtgcacacggtc

SEQ ID NO: 21 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
ccgtgtgcacttcgcttca

SEQ ID NO: 22 - RNA, 21 nt, synthetic construct
  source 1..21: mol_type = other RNA
  modified_base order(1,3,4,6,7,9..13,15,16,18..21): mod_base = OTHER; note = 2-prime-O-methyl RNA nucleotides
  modified_base order(2,5,8,14,17): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
tgaagcgaagtgcacacggtc

SEQ ID NO: 23 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
ccgtgtgcacttcgcttca

SEQ ID NO: 24 - RNA, 21 nt, synthetic construct
  source 1..21: mol_type = other RNA
  modified_base order(1,3,4,6,7,9..13,15,16,18..21): mod_base = OTHER; note = 2-prime-O-methyl RNA nucleotides
  modified_base order(2,5,8,14,17): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
tgaagcgaagtgcacacggtc

SEQ ID NO: 25 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
ccgtgtgcacttcgcttca

SEQ ID NO: 26 - RNA, 21 nt, synthetic construct
  source 1..21: mol_type = other RNA
  modified_base order(1,3,4,6,7,9..13,15,16,18..21): mod_base = OTHER; note = 2-prime-O-methyl RNA nucleotides
  modified_base order(2,5,8,14,17): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
tgaagcgaagtgcacacggtc

SEQ ID NO: 27 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
ccgtgtgcacttcgcttca

SEQ ID NO: 28 - RNA, 21 nt, synthetic construct
  source 1..21: mol_type = other RNA
  modified_base order(1,3,4,6,7,9..13,15,16,18..21): mod_base = OTHER; note = 2-prime-O-methyl RNA nucleotides
  modified_base order(2,5,8,14,17): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
tgaagcgaagtgcacacggtc

SEQ ID NO: 29 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
ccgtgtgcacttcgcttca

SEQ ID NO: 30 - RNA, 21 nt, synthetic construct
  source 1..21: mol_type = other RNA
  modified_base order(1,3,4,6,7,9..13,15,16,18..21): mod_base = OTHER; note = 2-prime-O-methyl RNA nucleotides
  modified_base order(2,5,8,14,17): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
tgaagcgaagtgcacacggtc

SEQ ID NO: 31-32 - skipped (2 entries)

SEQ ID NO: 33 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
gtggtggacttctctcaat

SEQ ID NO: 34 - RNA, 21 nt, synthetic construct
  source 1..21: mol_type = other RNA
  modified_base order(1,3..5,7..13,15,17..21): mod_base = OTHER; note = 2-prime-O-methyl RNA nucleotides
  modified_base order(2,6,14,16): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
attgagagaagtccancacga

SEQ ID NO: 35 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
ccgtgtgcacttcgcttca

SEQ ID NO: 36 - RNA, 21 nt, synthetic construct
  source 1..21: mol_type = other RNA
  modified_base order(1,3,4,7,9..13,15,16,18..21): mod_base = OTHER; note = 2-prime-O-methyl RNA nucleotides
  modified_base order(2,5,8,14,17): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
  modified_base 6: mod_base = OTHER; note = 3-prime,4-prime seco modified nucleotide
tgaagcgaagtgcacacggtc

SEQ ID NO: 37 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
ccgtgtgcacttcgcttca

SEQ ID NO: 38 - RNA, 21 nt, synthetic construct
  source 1..21: mol_type = other RNA
  modified_base order(1,3,4,6,9..13,15,16,18..21): mod_base = OTHER; note = 2-prime-O-methyl RNA nucleotides
  modified_base order(2,5,8,14,17): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
  modified_base 7: mod_base = OTHER; note = 3-prime,4-prime seco modified nucleotide
tgaagcgaagtgcacacggtc

SEQ ID NO: 39 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
ccgtgtgcacttcgcttca

SEQ ID NO: 40 - RNA, 21 nt, synthetic construct
  source 1..21: mol_type = other RNA
  modified_base order(1,3,4,6,9..13,15,16,18..21): mod_base = OTHER; note = 2-prime-O-methyl RNA nucleotides
  modified_base order(2,5,8,14,17): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
  modified_base 7: mod_base = OTHER; note = 3-prime,4-prime seco modified nucleotide
tgaagcgaagtgcacacggtc

SEQ ID NO: 41 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
gtggtggacttctctcaat

SEQ ID NO: 42 - RNA, 21 nt, synthetic construct
  source 1..21: mol_type = other RNA
  modified_base order(1,3..5,7..13,15,17..21): mod_base = OTHER; note = 2-prime-O-methyl RNA nucleotides
  modified_base order(2,6,14,16): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
attgagagaagtccaccacga

SEQ ID NO: 43 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
ccgtgtgcacttcgcttca

SEQ ID NO: 44 - RNA, 21 nt, synthetic construct
  source 1..21: mol_type = other RNA
  modified_base order(1,3,4,6,7,9..13,15,16,18..21): mod_base = OTHER; note = 2-prime-O-methyl RNA nucleotides
  modified_base order(2,5,8,14,17): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
tgaagcgaagtgcacacggtc

SEQ ID NO: 45 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
ccgtgtgcacttcgcttca

SEQ ID NO: 46 - RNA, 21 nt, synthetic construct
  source 1..21: mol_type = other RNA
  modified_base order(1,3,4,6,7,9..13,15,16,18..21): mod_base = OTHER; note = 2-prime-O-methyl RNA nucleotides
  modified_base order(2,5,8,14,17): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
tgaagcgaagtgcacacggtc

SEQ ID NO: 47 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
ccgtgtgcacttcgcttca

SEQ ID NO: 48 - RNA, 21 nt, synthetic construct
  source 1..21: mol_type = other RNA
  modified_base order(1,3,4,6,7,9..13,15,16,18..21): mod_base = OTHER; note = 2-prime-O-methyl RNA nucleotides
  modified_base order(2,5,8,14,17): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
tgaagcgaagtgcacacggtc

SEQ ID NO: 49 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
ccgtgtgcacttcgcttca

SEQ ID NO: 50 - RNA, 21 nt, synthetic construct
  source 1..21: mol_type = other RNA
  modified_base order(1,3,4,6,7,9..13,15,16,18..21): mod_base = OTHER; note = 2-prime-O-methyl RNA nucleotides
  modified_base order(2,5,8,14,17): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
tgaagcgaagtgcacacggtc

SEQ ID NO: 51 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
ccgtgtgcacttcgcttca

SEQ ID NO: 52 - RNA, 21 nt, synthetic construct
  source 1..21: mol_type = other RNA
  modified_base order(1,3,4,6,7,9..13,15,16,18..21): mod_base = OTHER; note = 2-prime-O-methyl RNA nucleotides
  modified_base order(2,5,8,14,17): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
tgaagcgaagtgcacacggtc

SEQ ID NO: 53 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
ccgtgtgcacttcgcttca

SEQ ID NO: 54 - RNA, 21 nt, synthetic construct
  source 1..21: mol_type = other RNA
  modified_base order(1,3,4,6,7,9..13,15,16,18..21): mod_base = OTHER; note = 2-prime-O-methyl RNA nucleotides
  modified_base order(2,5,8,14,17): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
tgaagcgaagtgcacacggtc

SEQ ID NO: 55 - DNA, 3182 bp, Hepatitis B virus
  source 1..3182: mol_type = other DNA
aattccacaacctttcaccaaactctgcaagatcccagagtgagaggcctgtatttccctgctggtggctccagttcaggagcagtaaaccctgttccgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcctgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaaccaccagcacgggaccatgccgaacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctgaattttatgggttatgtcattggaagttatgggtccttgccacaagaacacatcatacaaaaaatcaaagaatgttttagaaaacttcctattaacaggcctattgattggaaagtatgtcaacgaattgtgggtcttttgggttttgctgccccatttacacaatgtggttatcctgcgttaatgcccttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcgtgcgtggaaccttttcggctcctctgccgatccatactgcggaactcctagccgcttgttttgctcgcagcaggtctggagcaaacattatcgggactgataactctgttgtcctctcccgcaaatatacatcgtatccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccgaatgttgcccaaggtcttacataagaggactcttggactctctgcaatgtcaacgaccgaccttgaggcatacttcaaagactgtttgtttaaagactgggaggagttgggggaggagattagattaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcatctagagacctagtagtcagttatgtcaacactaatatgggcctaaagttcaggcaactcttgtggtttcacatttcttgtctcacttttggaagagaaaccgttatagagtatttggtgtctttcggagtgtggattcgcactcctccagcttatagaccaccaaatgcccctatcctatcaacacttccggaaactactgttgttagacgacgaggcaggtcccctagaagaagaactccctcgcctcgcagacgaaggtctcaatcgccgcgtcgcagaagatctcaatctcgggaacctcaatgttagtattccttggactcataaggtggggaactttactggtctttattcttctactgtacctgtctttaatcctcattggaaaacaccatcttttcctaatatacatttacaccaagacattatcaaaaaatgtgaacagtttgtaggcccacttacagttaatgagaaaagaagattgcaattgattatgcctgctaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcatggggcagaatctttccaccagcaatcctctgggattctttcccgaccaccagttggatccagccttcagagcaaacacagcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagacaggaaggcagcctaccccgctgtctccacctttgagaaacactcatcctcaggccatgcagtgg

SEQ ID NO: 56 - DNA, 7459 bp, synthetic construct
  source 1..7459: mol_type = other DNA
agtcactgccgcggcgccttgagtctccgggccgccttgccatggctgcccgtggtgtcatcgctccagttggcgagagtttgcgctacgctgagtacttgcagccctcggccaaacggccagacgccgacgtcgaccagcagagactggtaagaagtttgatagctgtaggactgggtgttgcagctcttgcatttgcaggtcgctacgcatttcggatctggaaacctctagaacaagttatcacagaaactgcaaagaagatttcaactcctagcttttcatcctactataaaggaggatttgaacagaaaatgagtaggcgagaagctggtcttattttaggtgtaagcccatctgctggcaaggctaagattagaacagctcataggagagtcatgattttgaatcacccagataaaggtggatctccttacgtagcagccaaaataaatgaagcaaaagacttgctagaaacaaccaccaaacattgatgcttaaggaccacactgaaggaaaaaaaaagaggggacttcgaaaaaaaaaaaagccctgcaaaatattctaaaacatggtcttcttaattttctatatggattgaccacagtcttatcttccaccattaagctgtataacaataaaatgttaatagtcttgctttttattatcttttaaagatctccttaaattctataactgatcttttttcttattttgtttgtgacattcatacatttttaagatttttgttatgttctgaattcccccctacacacacacacacacacacacacacacacacgtgcaaaaaatatgatcaagaatgcaattgggatttgtgagcaatgagtagacctcttattgtttatatttgtaccctcattgtcaatttttttttagggaatttgggactctgcctatataaggtgttttaaatgtcttgagaacaagcactggctgatacctcttggagatatgatctgaaatgtaatggaatttattaaatggtgtttagtaaagtaggggttaaggacttgttaaagaaccccactatctctgagaccctatagccaaagcatgaggacttggagagctactaaaatgattcaggtttacaaaatgagccctgtgaggaaaggttgagagaagtctgaggagtttgtatttaattatagtcttccagtactgtatattcattcattactcattctacaaatatttattgaccccttttgatgtgcaaggcactatcgtgcgtcccctgagagttgcaagtatgaagcagtcatggatcatgaaccaaaggaacttatatgtagaggaaggataaatcacaaatagtgaatactgttagatacagatgatatattttaaaagttcaaaggaagaaaagaatgtgttaaacactgcatgagaggaggaataagtggcatagagctaggctttagaaaagaaaaatattccgataccatatgattggtgaggtaagtgttattctgagatgagaattagcagaaatagatatatcaatcggagtgattagagtgcagggtttctggaaagcaaggtttggacagagtggtcatcaaaggccagccctgtgacttacactgcattaaattaatttcttagaacatagtccctgatcattatcactttactattccaaaggtgagagaacagattcagatagagtgccagcattgtttcccagtattcctttacaaatcttgggttcattccaggtaaactgaactactgcattgtttctatcttaaaatactttttagatatcctagatgcatctttcaacttctaacattctgtagtttaggagttctcaaccttggcattattgacatgttaggccaaataattttttttgtgggaggtctcttgtgcgttttagatgattagcaataatccctgacctgttatctactaaagactagtcgtttctcatcagttgtgacaacaaaaatggttccagatattgccaaatgccctttagaggacagtaatcgcccccagttgagaaccatttcagtaaaactttaattactattttttcttttggtttataaaataatgatcctgaattaaattgatggaaccttgaagtcgataaaatatatttcttgctttaaagtccccatacgtgtcctactaattttctcatgctttagtgttttcacttttctcctgttatccttgtacctaagaatgccatcccaatccccagatgtccacctgcccaaagtctaggcatagctgaaggccaagctaaaatgtatccctctttttctggtacatgcagcaaaagtaatatgaattatcagctttctgagagcaggcattgtatctgtcttgtttggtgttacattggcacccaataaatatttgttgagtgaatgaataaattcccatagcactttattcttcacatggtacataactataggggctatagcttggtaccttgtgaagcaactcttggtgtaacataccttatttctcatactaaaatgcaagaacctttagagcaaggatcttgccattcatctttgtaacctctttactctggagcacttgcatttagcaggcatcataaagttttacgtaccaagaaaatgttgctgttttctgaatactatgcatcaaaaaatgttaccactaatttttaaagctctgctaaggaatattggggcaccctcagatgcaccttttaattgatgtcatattttcctaatccatactttattcatgagaatttgagtcaccccagcattagcttggaatttccttatttcccatttgctttgcaggtgccttggagtcagatctggttttgaatactatcttcctgttatgtgatcttgggcagttacttaattttctagtcaataacccgtatctataaaatagagaaaataatcctacacaccggggcctgttgtggggcggggagaggggggagggatcgcatttggagatatactaatgtaaatgacaagttaattggtgcagcacaccaacatggctcatgtctacatatgtaacaaacctgcacgttgtgcacatgtgccctagaacttaaagtataataaaaagaaattttaaaaaatcctgtcaaataaggttatagtagagaataaggatgtgtaaagcatttagtcacgtaaatgcttaaaaaaatgtaatttttacttctttcactgcctcatttaattagttttatctttaataataccttggattcagggtaaagtttcagttatgtcccagtaatcatttattttaccctcgaatctgcaatttggatagaacatggtggggacagctcgtctctattccttgcagcattaacaggctggaggcaccacttctctggccagcaagttgggcctggttgttggctgagagcctcagttcctttctgcacaggttcctctttacataggcttctcaacagggctactagagcatcgtcaccatagcagctgtcttataacagagagtggtcggtctgagagacaaaaaatggaagctgccaaattgttctgggtctggaaactgtcagggcatcacttgtgccatattcagttggcctaagaattacagagcctgcctcgattcaaagggagaggatagagaggactgaaggaatcagtgctcatctttaatatgcagcaggacaggtttgggattttttttcccccttgagtctgtgaaggcattacttaagaacaaagtcaggcatgtataattgaactacagttacttgaaatataagcccagaaagtttcagataataaatacaactatttttctgctgttacccttgtacctaaagatgccatcctaatccccagatctccacaactatacctacatagtagaaggttaaaatgtatccctctttttctggtgcatccagcaaaagtaatatcatgaattatgagctctctgagagcaaggatcatatcagtcttgtttattgttgcagtgaacaagtacagttgcagatattcaggagtaattatctaaatggcagtaggcttataaaactgaattttcaccagccacaccctccccccaactccttatctgtaaaaagcttatttgagtggttacctgtcttcagtaaagattgcgcttgcatatttgctgtcattgcatattctgcttaattaagctctgttgatattgcagtttctgtgcatacttacatcttagatgcaatctgagggcctaggaaggccttttaaaaataaaacaattccgattgcagagaaagtgtaagtcaaggacagttaattcaaggggaacatagaaagctatttagattttagttgatggtgccagtcttcagcgtaaagtcaaaagtggagggaagtttagtaaggaaaaaatgttgggcttggaatacattgtttagtcttcaaagcactttactttttatgaaatatattttagacattcagcaaatattgaatacttactatatcaggcagtaaagatataaattcattcttaaaatgtgcaacatgttcaaactgaaaaaaatacattcttaaacaggaaactttttccttcatactttttaattaacaagacatataagagttgcattaatgggcgtgcttatgattgatcacccagcagcatcattagaaataatatattttattcatgtgcagaaatcttttggttgtcctggggaaccttgaacacagaaaagagcttttattgataaggtaattgaacacacttgacaattagcttaatatggtttaataccatttgtgggagaagatgaatcagccaggctctttacgtcaagaatatgaagtttctcttgagtcaaccaacttaagatgagctacggagactgcagtgaaaagttaaatatccaagtacaccagccaatttcacacagtggaaccatgctgtcctcgggcaccctgcacctcgcccaacagtcatcaactagatggaggctcctggctgcaaggaggatttgatgggaatgagtaaatgtgtcagcatagtccgtcccttctaatggaaaagcaacccaaagagcaaatcctattaatggctggatcagtatcatctacttgtcaaaaacattccatgaattatgagtcaaaattttatttatggtggcattacacacattaagagatgaggacttctgttagcataatttattagctggaaaagttgagaaggttctctggactcatttttataggtggaacctaagtgatctggataattgcccaccagcaaaattgctgggcatggtggacaaagaaaatgttccttctaatgattttttatgagctgagtagctattgttcccagctgagtgctcttttcctctttttattgttgctgagcaaaagaatttataaaaagctctttcttttgtattaaaaaccctgctcaattgaaatgcaagttcattaagtaatcttcatttctcttcctgccataataaccctttccctctctgttcgattcaacagtatctagcagcactgctccaaattttaagtctgaacagactatattacatagatgtagagaaatactcaatcttcagcattaagagggagcttaatttcacacgggtggaatatgatcactcaggctagatgttggccataaatttcaaattagtatctcaacttagcaggggggatcaacagtggcaaacttcaattatgacaggataaaaatcacatagagatattggttcaatatggacatctaaactataatgctaaaagccaataattagaataagttcattttaagaaaagcattaataatattagctaacgtttagtacctgtgccaaacattctacctatgttaccttgattttcatagccagcctaagaggtactattatgtatccccattttacaggttaagaaacaggctcagaggagtttaggatcttttccaagattacatagccagtaagtggtggcactaggaaccaaattcagactctgaatcgcatgctgtttatattatattgcactcattctaaatatgtgggaatcagaatgaaggggcttgtatgacttttggctcattttttgatgcatgtgacctgggattataaatgtgaaattaggtttacgaaaggatccagtgtcattgtgcatcatgggcaaggagtacctaatctctttaattcttccctggaagcttacgatgtccatccaagtgcacatagcaaaagttctgttgtaaagtttagcagagtgactttctttgactcagagtgatgacggaggaagctttgataagattttatctgaaatgttcatggacaagagctttcaaggagaacatccagagcaaggttctgaagacagctcatgaaggtgaagcagcagacctggcacaagaaatgaagagagagctcagtgtattaaagatgaaaacaagaaaaccgaatatattgaaaggagcagagaggcaatgaaaacaagacaactgaaatgaggtaacttgcagcaattgaaagggaatttcagtacttttatagaattcttaaaaattgtttcctgctgtttattttcaattttgaacagggttatttgtccatgccatactttttttgccaaattccaaaattgtgtatagttctatagttgtctggtggagtcaatggaactttagttaccagtctaagaatgtgtctttgagattgtccagttaattctctatttccagtagctgtaataaatggtgaaaaggtttctgactcctggagaaagtttctaactccttatgactaatattcataacagacttgtgagttccttgaacatggatacacctatatgcaagagtgtattccaaagctaactcagtgatctttccatttatctattcttggattagtggtgcctttgctctttccttctgtaaatgtgaatagttaagagttgactgcagaagtgtttacactttggcttccatgcctctggaatgtttgtgctttggtggtgagatgtgagactatatttgtatagtctgcatctctcaggctgccccagaatgttgtacagtgcagtgctgaagaaagcagcaggtacacacagaaatgcagcctttcctggttaaccctgcttggatctgagttacactttgtttcctgacttcttgggacttaggtaatcagtttgccttctactctatctcattttgtactcgcttacatactacattcttgtttgggctttcgtttcttcttgtaagcagagattttttaaaatccaatatgtgaaaatacggatgcactacaattaaataaataaaatgctgttgtgtttgttttgctttaaaattgtaaaggataaacaataagatagttttatctatgtggttttcccgatgcagttaaaataaaacctaatctgctaaaattgaa

SEQ ID NO: 57 - DNA, 1906 bp, synthetic construct
  source 1..1906: mol_type = other DNA
gctttccggcggttgcaccgggccggggtgccagcgcccgccttcccgtttcctcccgttccgcagcgcgcccacggcctgtgaccccggcgaccgctccccagtgacgagagagcggggccgggcgctgctccggcctgacctgcgaagggacctcggtccagtcccctgttgcgccgcgcccccgtccgtccgtgcgcgggccagtcaggggccagtgtctcgagcggtcgaggtcgcagacctagaggcgccccacaggccggcccggggcgctgggagcgccggccgcgggccgggtggggatgcctctgcacgtgaagtggccgttccccgcggtgccgccgctcacctggaccctggccagcagcgtcgtcatgggcttggtgggcacctacagctgcttctggaccaagtacatgaaccacctgaccgtgcacaacagggaggtgctgtacgagctcatcgagaagcgaggcccggccacgcccctcatcaccgtgtccaatcaccagtcctgcatggacgaccctcatctctgggggatcctgaaactccgccacatctggaacctgaagttgatgcgttggacccctgcagctgcagacatctgcttcaccaaggagctacactcccacttcttcagcttgggcaagtgtgtgcctgtgtgccgaggagcagaatttttccaagcagagaatgaggggaaaggtgttctagacacaggcaggcacatgccaggtgctggaaaaagaagagagaaaggagatggcgtctaccagaaggggatggacttcattttggagaagctcaaccatggggactgggtgcatatcttcccagaagggaaagtgaacatgagttccgaattcctgcgtttcaagtggggaatcgggcgcctgattgctgagtgtcatctcaaccccatcatcctgcccctgtggcatgtcggaatgaatgacgtccttcctaacagtccgccctacttcccccgctttggacagaaaatcactgtgctgatcgggaagcccttcagtgccctgcctgtactcgagcggctccgggcggagaacaagtcggctgtggagatgcggaaagccctgacggacttcattcaagaggaattccagcatctgaagactcaggcagagcagctccacaaccacctccagcctgggagataggccttgcttgctgccttctggattcttggcccgcacagagctggggctgagggatggactgatgcttttagctcaaacgtggcttttagacagatttgttcatagaccctctcaagtgccctctccgagctggtaggcattccagctcctccgtgcttcctcagttacacaaaggacctcagctgcttctcccacttggccaagcagggaggaagaagcttaggcagggctctctttccttcttgccttcagatgttctctcccaggggctggcttcaggagggagcatagaaggcaggtgagcaaccagttggctaggggagcagggggcccaccagagctgtggagaggggaccctaagactcctcggcctggctcctacccaccgcccttgccgaaccaggagctgctcactacctcctcagggatggccgttggccacgtcttccttctgcctgagcttcccccccaccacaggccctttcctcaggcaaggtctggcctcaggtgggccgcaggcgggaaaagcagcccttggccagaagtcaagcccagccacgtggagcctagagtgagggcctgaggtctggctgcttgcccccatgctggcgccaacaacttctccatcctttctgcctctcaacatcacttgaatcctagggcctgggttttcatgtttttgaaacagaaccataaagcatatgtgttggcttgttgtaaaa

SEQ ID NO: 58 - DNA, 2926 bp, synthetic construct
  source 1..2926: mol_type = other DNA
agaagaaaacagttccacgttgcttgaaattgaaaatcaagataaaaatgttcacaattaagctccttctttttattgttcctctagttatttcctccagaattgatcaagacaattcatcatttgattctctatctccagagccaaaatcaagatttgctatgttagacgatgtaaaaattttagccaatggcctccttcagttgggacatggtcttaaagactttgtccataagacgaagggccaaattaatgacatatttcaaaaactcaacatatttgatcagtctttttatgatctatcgctgcaaaccagtgaaatcaaagaagaagaaaaggaactgagaagaactacatataaactacaagtcaaaaatgaagaggtaaagaatatgtcacttgaactcaactcaaaacttgaaagcctcctagaagaaaaaattctacttcaacaaaaagtgaaatatttagaagagcaactaactaacttaattcaaaatcaacctgaaactccagaacacccagaagtaacttcacttaaaacttttgtagaaaaacaagataatagcatcaaagaccttctccagaccgtggaagaccaatataaacaattaaaccaacagcatagtcaaataaaagaaatagaaaatcagctcagaaggactagtattcaagaacccacagaaatttctctatcttccaagccaagagcaccaagaactactccctttcttcagttgaatgaaataagaaatgtaaaacatgatggcattcctgctgaatgtaccaccatttataacagaggtgaacatacaagtggcatgtatgccatcagacccagcaactctcaagtttttcatgtctactgtgatgttatatcaggtagtccatggacattaattcaacatcgaatagatggatcacaaaacttcaatgaaacgtgggagaactacaaatatggttttgggaggcttgatggagaattttggttgggcctagagaagatatactccatagtgaagcaatctaattatgttttacgaattgagttggaagactggaaagacaacaaacattatattgaatattctttttacttgggaaatcacgaaaccaactatacgctacatctagttgcgattactggcaatgtccccaatgcaatcccggaaaacaaagatttggtgttttctacttgggatcacaaagcaaaaggacacttcaactgtccagagggttattcaggaggctggtggtggcatgatgagtgtggagaaaacaacctaaatggtaaatataacaaaccaagagcaaaatctaagccagagaggagaagaggattatcttggaagtctcaaaatggaaggttatactctataaaatcaaccaaaatgttgatccatccaacagattcagaaagctttgaatgaactgaggcaaatttaaaaggcaataatttaaacattaacctcattccaagttaatgtggtctaataatctggtattaaatccttaagagaaagcttgagaaatagattttttttatcttaaagtcactgtctatttaagattaaacatacaatcacataaccttaaagaataccgtttacatttctcaatcaaaattcttataatactatttgttttaaattttgtgatgtgggaatcaattttagatggtcacaatctagattataatcaataggtgaacttattaaataacttttctaaataaaaaatttagagacttttattttaaaaggcatcatatgagctaatatcacaactttcccagtttaaaaaactagtactcttgttaaaactctaaacttgactaaatacagaggactggtaattgtacagttcttaaatgttgtagtattaatttcaaaactaaaaatcgtcagcacagagtatgtgtaaaaatctgtaatacaaatttttaaactgatgcttcattttgctacaaaataatttggagtaaatgtttgatatgatttatttatgaaacctaatgaagcagaattaaatactgtattaaaataagttcgctgtctttaaacaaatggagatgactactaagtcacattgactttaacatgaggtatcactataccttatttgttaaaatatatactgtatacattttatatattttaacacttaatactatgaaaacaaataattgtaaaggaatcttgtcagattacagtaagaatgaacatatttgtggcatcgagttaaagtttatatttcccctaaatatgctgtgattctaatacattcgtgtaggttttcaagtagaaataaacctcgtaacaagttactgaacgtttaaacagcctgacaagcatgtatatatgtttaaaattcaataaacaaagacccagtccctaaattatagaaatttaaattattcttgcatgtttatcgacatcacaacagatccctaaatccctaaatccctaaagattagatacaaattttttaccacagtatcacttgtcagaatttatttttaaatatgattttttaaaactgccagtaagaaattttaaattaaacccatttgttaaaggatatagtgcccaagttatatggtgacctacctttgtcaatacttagcattatgtatttcaaattatccaatatacatgtcatatatatttttatatgtcacatatataaaagatatgtatgatctatgtgaatcctaagtaaatattttgttccagaaaagtacaaaataataaaggtaaaaataatctataattttcaggaccacagactaagctgtcgaaattaacgctgatttttttagggccagaataccaaaatggctcctctcttcccccaaaattggacaatttcaaatgcaaaataattcattatttaatatatgagttgcttcctctatttggtttcc

SEQ ID NO: 59 - DNA, 2336 bp, synthetic construct
  source 1..2336: mol_type = other DNA
tgccccgttgtgaggtgataaagtgttgcgctccgggacgccagcgccgcggctgccgcctctgctggggtctaggctgtttctctcgcgccaccactggccgccggccgcagctccaggtgtcctagccgcccagcctcgacgccgtcccgggacccctgtgctctgcgcgaagccctggccccgggggccggggcatgggccaggggcgcggggtgaagcggcttcccgcggggccgtgactgggcgggcttcagccatgaagaccctcatagccgcctactccggggtcctgcgcggcgagcgtcaggccgaggctgaccggagccagcgctctcacggaggacctgcgctgtcgcgcgaggggtctgggagatggggagtggcctgcagtgccatcctcatgtacatattctgcactgattgctggctcatcgctgtgctctacttcacttggctggtgtttgactggaacacacccaagaaaggtggcaggaggtcacagtgggtccgaaactgggctgtgtggcgctactttcgagactactttcccatccagctggtgaagacacacaacctgctgaccaccaggaactatatctttggataccacccccatggtatcatgggcctgggtgccttctgcaacttcagcacagaggccacagaagtgagcaagaagttcccaggcatacggccttacctggctacactggcaggcaacttccgaatgcctgtgttgagggagtacctgatgtctggaggtatctgccctgtcagccgggacaccatagactatttgctttcaaagaatgggagtggcaatgctatcatcatcgtggtcgggggtgcggctgagtctctgagctccatgcctggcaagaatgcagtcaccctgcggaaccgcaagggctttgtgaaactggccctgcgtcatggagctgacctggttcccatctactcctttggagagaatgaagtgtacaagcaggtgatcttcgaggagggctcctggggccgatgggtccagaagaagttccagaaatacattggtttcgccccatgcatcttccatggtcgaggcctcttctcctccgacacctgggggctggtgccctactccaagcccatcaccactgttgtgggagagcccatcaccatccccaagctggagcacccaacccagcaagacatcgacctgtaccacaccatgtacatggaggccctggtgaagctcttcgacaagcacaagaccaagttcggcctcccggagactgaggtcctggaggtgaactgagccagccttcggggccaattccctggaggaaccagctgcaaatcacttttttgctctgtaaatttggaagtgtcatgggtgtctgtgggttatttaaaagaaattataacaattttgctaaaccattacaatgttaggtcttttttaagaaggaaaaagtcagtatttcaagttctttcacttccagcttgccctgttctaggtggtggctaaatctgggcctaatctgggtggctcagctaacctctcttcttcccttcctgaagtgacaaaggaaactcagtcttcttggggaagaaggattgccattagtgacttggaccagttagatgattcactttttgcccctagggatgagaggcgaaagccacttctcatacaagcccctttattgccactaccccacgctcgtctagtcctgaaactgcaggaccagtttctctgccaaggggaggagttggagagcacagttgccccgttgtgtgagggcagtagtaggcatctggaatgctccagtttgatctcccttctgccacccctacctcacccctagtcactcatatcggagcctggactggcctccaggatgaggatgggggtggcaatgacaccctgcaggggaaaggactgccccccatgcaccattgcagggaggatgccgccaccatgagctaggtggagtaactggtttttcttgggtggctgatgacatggatgcagcacagactcagccttggcctggagcacatgcttactggtggcctcagtttaccttccccagatcctagattctggatgtgaggaagagatccctcttcagaaggggcctggccttctgagcagcagattagttccaaagcaggtggcccccgaacccaagcctcacttttctgtgccttcctgagggggttgggccggggaggaaacccaaccctctcctgtgtgttctgttatctcttgatgagatcattgcaccatgtcagacttttgtatatgccttgaaaataaatgaaagtgagaatcctctaaaaaaaaaaaa

SEQ ID NO: 60 - DNA, 3215 bp, synthetic construct
  source 1..3215: mol_type = other DNA
ctccaccactttccaccaaactcttcaagatcccagagtcagggccctgtactttcctgctggtggctcaagttccggaacagtaaaccctgctccgactactgcctctcccatatcgtcaatcttctcgaggactggggaccctgtaccgaatatggagagcaccacatcaggattcctaggacccctgctcgtgttacaggcggggtttttcttgttgacaagaatcctcacaataccacagagtctagactcgtggtggacttctctcaattttctagggggagcacccacgtgtcctggccaaaatttgcagtccccaacctccaatcactcaccaacctcttgtcctccaatttgtcctggttatcgctggatgtgtctgcggcgttttatcatcttcctcttcatcctgctgctatgcctcatcttcttgttggttcttctggactaccaaggtatgttgcccgtttgtcctctacttccaggaacatcaactaccagcaccggaccatgcaaaacctgcacaactactgctcaagggacctctatgtttccctcatgttgctgtacaaaacctacggacggaaactgcacctgtattcccatcccatcatcttgggctttcgcaaaatacctatgggagtgggcctcagtccgtttctcttggctcagtttactagtgccatttgttcagtggttcgtagggctttcccccactgtctggctttcagttatatggatgatgtggttttgggggccaagtctgtacaacatcttgagtccctttataccgctgttaccaattttcttttatctttgggtatacatttaaaccctcacaaaacaaaaagatggggatattcccttaacttcatgggatatgtaattgggagttggggcactttgcctcaggaacatattgtacaaaaaatcaagcaatgttttaggaaacttcctgtaaacaggcctattgattggaaagtatgtcaacraattgtgggtcttttggggtttgccgcccctttcacgcaatgtggatatcctgctttaatgcctttatatgcatgtatacaagctaagcaggcttttactttctcgccaacttacaaggcctttctgtgtaaacaatatctgaacctttaccccgttgctcggcaacggtcaggtctttgccaagtgtttgctgacgcaacccccactggttggggcttggccataggccatcagcgcatgcgtggaacctttgtggctcctctgccgatccatactgcggaactcctagcagcttgttttgctcgcagccggtctggagcaaaacttatcggcaccgacaactctgttgtcctctctcggaaatacacctcctttccatggctgctaggatgtgctgccaactggatcctgcgcgggacgtcctttgtctacgtcccgtcggcgctgaatcccgcggacgacccatctcggggccgtttgggactctaccgtccccttctgcgtctgccgttccgcccgaccacggggcgcacctctctttacgcggtctccccgtctgtgccttctcatctgccggaccgtgtgcacttcgcttcacctctgcacgtcgcatggagaccaccgtgaacgcccacgggaacctgcccaaggtcttgcataagaggactcttggactttcagcaatgtcaacgaccgaccttgaggcatacttcaaagactgtgtgtttactgagtgggaggagttgggggaggaggttaggttaaaggtctttgtactaggaggctgtaggcataaattggtgtgttcaccagcaccatgcaactttttcacctctgcctaatcatctcatgttcatgtcctactgttcaagcctccaagctgtgccttgggtggctttggggcatggacattgacccgtataaagaatttggagcttctgtggagttactctcttttttgccttctgacttctttccttctattcgagatctcctcgacaccgcctctgctctgtatcgggaggccttagagtctccggaacattgttcacctcaccatacggcactcaggcaagcaattctgtgttggggtgagttaatgaatctagccacctgggtgggaagtaatttggaagatccagcatccagggaattagtagtcagctatgtcaacgttaatatgggcctaaaaatcagacaactattgtggtttcacatttcctgtcttacttttgggagagaaactgttcttgaatatttggtgtcttttggagtgtggattcgcactcctcctgcatatagaccacaaaatgcccctatcttatcaacacttccggaaactactgttgttagacgaagaggcaggtcccctagaagaagaactccctcgcctcgcagacgaaggtctcaatcgccgcgtcgcagaagatctcaatctcgggaatctcaatgttagtattccttggacacataaggtgggaaactttacggggctttattcttctacggtaccttgctttaatcctaaatggcaaactccttcttttcctgacattcatttgcaggaggacattgttgatagatgtaagcaatttgtggggccccttacagtaaatgaaaacaggagacttaaattaattatgcctgctaggttttatcccaatgttactaaatatttgcccttagataaagggatcaaaccgtattatccagagtatgtagttaatcattacttccagacgcgacattatttacacactctttggaaggcggggatcttatataaaagagagtccacacgtagcgcctcattttgcgggtcaccatattcttgggaacaagatctacagcatgggaggttggtcttccaaacctcgaaaaggcatggggacaaatctttctgtccccaatcccctgggattcttccccgatcatcagttggaccctgcattcaaagccaactcagaaaatccagattgggacctcaacccacacaaggacaactggccggacgccaacaaggtgggagtgggagcattcgggccagggttcacccctcctcatgggggactgttggggtggagccctcaggctcagggcatattcacaacagtgccagcagctcctcctcctgcctccaccaatcggcagtcaggaaggcagcctactcccttctctccacctctaagagacactcatcctcaggccatgcagtggaa

SEQ ID NO: 61 - DNA, 18 bp, synthetic construct
  misc_feature 4: note = DNA
  misc_feature order(1..3,5..18): note = RNA
  source 1..18: mol_type = other DNA
  modified_base 1: mod_base = OTHER; note = 2-prime-O-methyl RNA nucleotide
  modified_base order(2..6,15..17): mod_base = OTHER; note = Locked nucleic acids
  modified_base 9: mod_base = OTHER; note = (5OH)C
  modified_base order(11,12,14): mod_base = OTHER; note = 5 methyl cytosine nucleotides
  modified_base 18: mod_base = OTHER; note = Cyclopropyl-5 methyl Cytosine nucleotide
  modified_base 1^2: mod_base = OTHER; note = phosphodiester
  modified_base 2^3: mod_base = OTHER; note = phosphorothioate
  modified_base 3^4: mod_base = OTHER; note = phosphorothioate
  modified_base 4^5: mod_base = OTHER; note = phosphorothioate
  modified_base 5^6: mod_base = OTHER; note = phosphorothioate
  modified_base 6^7: mod_base = OTHER; note = phosphorothioate
  modified_base 7^8: mod_base = OTHER; note = phosphorothioate
  modified_base 8^9: mod_base = OTHER; note = phosphorothioate
  modified_base 9^10: mod_base = OTHER; note = phosphorothioate
  modified_base 10^11: mod_base = OTHER; note = phosphorothioate
  modified_base 11^12: mod_base = OTHER; note = phosphorothioate
  modified_base 12^13: mod_base = OTHER; note = phosphorothioate
  modified_base 13^14: mod_base = OTHER; note = phosphorothioate
  modified_base 15^16: mod_base = OTHER; note = phosphorothioate
  modified_base 14^15: mod_base = OTHER; note = phosphorothioate
  modified_base 16^17: mod_base = OTHER; note = phosphorothioate
  modified_base 17^18: mod_base = OTHER; note = phosphorothioate
agataaaacgccgcagac

SEQ ID NO: 62 - DNA, 18 bp, synthetic construct
  misc_feature 4: note = DNA
  misc_feature order(1..3,5..18): note = RNA
  source 1..18: mol_type = other DNA
  modified_base 1: mod_base = OTHER; note = 2-prime-O-methyl RNA nucleotide
  modified_base order(2..6,15..17): mod_base = OTHER; note = Locked nucleic acids
  modified_base 9: mod_base = OTHER; note = (5OH)C
  modified_base order(11,12,14): mod_base = OTHER; note = 5 methyl cytosine nucleotides
  modified_base 18: mod_base = OTHER; note = Cyclopropyl-5 methyl Cytosine nucleotide
  modified_base 1^2: mod_base = OTHER; note = phosphodiester
  modified_base 2^3: mod_base = OTHER; note = phosphorothioate
  modified_base 3^4: mod_base = OTHER; note = phosphorothioate
  modified_base 4^5: mod_base = OTHER; note = phosphorothioate
  modified_base 5^6: mod_base = OTHER; note = phosphorothioate
  modified_base 6^7: mod_base = OTHER; note = phosphorothioate
  modified_base 7^8: mod_base = OTHER; note = phosphorothioate
  modified_base 8^9: mod_base = OTHER; note = phosphorothioate
  modified_base 9^10: mod_base = OTHER; note = phosphorothioate
  modified_base 10^11: mod_base = OTHER; note = phosphorothioate
  modified_base 11^12: mod_base = OTHER; note = phosphorothioate
  modified_base 12^13: mod_base = OTHER; note = phosphorothioate
  modified_base 13^14: mod_base = OTHER; note = phosphorothioate
  modified_base 15^16: mod_base = OTHER; note = phosphorothioate
  modified_base 14^15: mod_base = OTHER; note = phosphorothioate
  modified_base 16^17: mod_base = OTHER; note = phosphorothioate
  modified_base 17^18: mod_base = OTHER; note = phosphorothioate
agataaaacgccgcagac

SEQ ID NO: 63 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
ccgtgtgnacttcgcttca

SEQ ID NO: 64 - RNA, 21 nt, synthetic construct
  source 1..21: mol_type = other RNA
  modified_base order(1,3,4,6,7,9..13,15,16,18..21): mod_base = OTHER; note = 2-prime-O-methyl RNA nucleotides
  modified_base order(2,5,8,14,17): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
tgaagcgaagtgcacacggtc

SEQ ID NO: 65 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
ccgtgtgcacttcgcttca

SEQ ID NO: 66 - RNA, 21 nt, synthetic construct
  source 1..21: mol_type = other RNA
  modified_base order(1,3,4,7,9..13,15,16,18..21): mod_base = OTHER; note = 2-prime-O-methyl RNA nucleotides
  modified_base order(2,5,6,8,14,17): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
tgaagngaagtgcacacggtc

SEQ ID NO: 67 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
ccgtgtgcacttcgcttca

SEQ ID NO: 68 - RNA, 21 nt, synthetic construct
  source 1..21: mol_type = other RNA
  modified_base order(1,3,4,6,7,9..13,15,16,18..21): mod_base = OTHER; note = 2-prime-O-methyl RNA nucleotides
  modified_base order(2,5,8,14,17): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
tgaagcgaagtgcacanggtc

SEQ ID NO: 69 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
gtggtggacttctctcaat

SEQ ID NO: 70 - RNA, 21 nt, synthetic construct
  source 1..21: mol_type = other RNA
  modified_base order(1,3..5,7..13,15,17..21): mod_base = OTHER; note = 2-prime-O-methyl RNA nucleotides
  modified_base order(2,6,14,16): mod_base = OTHER; note = 2-prime-fluoro RNA nucleotides
  modified_base 1^2: mod_base = OTHER; note = mesyl phosphoramidate
  modified_base 2^3: mod_base = OTHER; note = mesyl phosphoramidate
  modified_base 19^20: mod_base = OTHER; note = phosphorothioate
  modified_base 20^21: mod_base = OTHER; note = phosphorothioate
attgagagaagtccaccacga

SEQ ID NO: 71 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
gtggtggacttctctcaat

SEQ ID NO: 72 - RNA, 21 nt, synthetic construct
  source 1..21: mol_type = other RNA
  modified_base order(1,3..5,7..13,15,17..21): mod_base = OTHER; note = 2-prime-O-methyl RNA nucleotides
  modified_base order(2,6,14,16): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = mesyl phosphoramidate
  modified_base 20^21: mod_base = OTHER; note = mesyl phosphoramidate
attgagagaagtccaccacga

SEQ ID NO: 73 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
gtggtggacttctctcaat

SEQ ID NO: 74 - RNA, 21 nt, synthetic construct
  source 1..21: mol_type = other RNA
  modified_base order(1,3..5,7..13,15,17..21): mod_base = OTHER; note = 2-prime-O-methyl RNA nucleotides
  modified_base order(2,6,14,16): mod_base = OTHER; note = 2-prime-fluoro RNA nucleotides
  modified_base 1^2: mod_base = OTHER; note = mesyl phosphoramidate
  modified_base 2^3: mod_base = OTHER; note = mesyl phosphoramidate
  modified_base 19^20: mod_base = OTHER; note = mesyl phosphoramidate
  modified_base 20^21: mod_base = OTHER; note = mesyl phosphoramidate
agcaccacctgaagagagtta

SEQ ID NO: 75 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
gtggtggacttctctcaat

SEQ ID NO: 76 - RNA, 21 nt, synthetic construct
  source 1..21: mol_type = other RNA
  modified_base order(1,3..5,7..13,15,17..21): mod_base = OTHER; note = 2-prime-O-methyl RNA nucleotides
  modified_base order(2,6,14,16): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
agcaccacctgaagagagtta

SEQ ID NO: 77 - RNA, 19 nt, synthetic construct
  source 1..19: mol_type = other RNA
  modified_base order(1..3,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 4: mod_base = OTHER; note = apU nucleotide
ccgtgtgcacttcgcttca

SEQ ID NO: 78 - RNA, 21 nt, synthetic construct
  source 1..21: mol_type = other RNA
  modified_base order(1,3,4,6,7,9..13,15,16,18..21): mod_base = OTHER; note = 2-prime-O-methyl RNA nucleotides
  modified_base order(2,5,8,14,17): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
tgaagcgaagtgcacacggtc

SEQ ID NO: 79 - RNA, 19 nt, synthetic construct
  source 1..19: mol_type = other RNA
  modified_base order(1..4,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 6: mod_base = OTHER; note = apU nucleotide
ccgtgtgcacttcgcttca

SEQ ID NO: 80 - RNA, 21 nt, synthetic construct
  source 1..21: mol_type = other RNA
  modified_base order(1,3,4,6,7,9..13,15,16,18..21): mod_base = OTHER; note = 2-prime-O-methyl RNA nucleotides
  modified_base order(2,5,8,14,17): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
tgaagcgaagtgcacacggtc

SEQ ID NO: 81 - RNA, 19 nt, synthetic construct
  source 1..19: mol_type = other RNA
  modified_base order(1..4,6,10,11,13..19): mod_base = OTHER; note = 2-prime-O-methyl RNA nucleotides
  modified_base order(5,7..9): mod_base = OTHER; note = 2-prime-fluoro RNA nucleotides
  modified_base 12: mod_base = OTHER; note = apU nucleotide
  modified_base 1^2: mod_base = OTHER; note = phosphorothioate
  modified_base 2^3: mod_base = OTHER; note = phosphorothioate
ccgtgtgcacttcgcttca

SEQ ID NO: 82 - RNA, 21 nt, synthetic construct
  source 1..21: mol_type = other RNA
  modified_base order(1,3,4,6,7,9..13,15,16,18..21): mod_base = OTHER; note = 2-prime-O-methyl RNA nucleotides
  modified_base order(2,5,8,14,17): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
tgaagcgaagtgcacacggtc

SEQ ID NO: 83 - RNA, 19 nt, synthetic construct
  source 1..19: mol_type = other RNA
  modified_base order(1..4,6,10..15,17..19): mod_base = OTHER; note = 2-prime-O-methyl RNA nucleotides
  modified_base order(5,7..9): mod_base = OTHER; note = 2-prime-fluoro RNA nucleotides
  modified_base 16: mod_base = OTHER; note = apU nucleotide
  modified_base 1^2: mod_base = OTHER; note = phosphorothioate
  modified_base 2^3: mod_base = OTHER; note = phosphorothioate
ccgtgtgcacttcgcttca

SEQ ID NO: 84 - RNA, 21 nt, synthetic construct
  source 1..21: mol_type = other RNA
  modified_base order(1,3,4,6,7,9..13,15,16,18..21): mod_base = OTHER; note = 2-prime-O-methyl RNA nucleotides
  modified_base order(2,5,8,14,17): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
tgaagcgaagtgcacacggtc

SEQ ID NO: 85 - RNA, 19 nt, synthetic construct
  source 1..19: mol_type = other RNA
  modified_base order(1..4,6,10..16,18,19): mod_base = OTHER; note = 2-prime-O-methyl RNA nucleotides
  modified_base order(5,7..9): mod_base = OTHER; note = 2-prime-fluoro RNA nucleotides
  modified_base 17: mod_base = OTHER; note = apU nucleotide
  modified_base 1^2: mod_base = OTHER; note = phosphorothioate
  modified_base 2^3: mod_base = OTHER; note = phosphorothioate
ccgtgtgcacttcgcttca

SEQ ID NO: 86 - RNA, 21 nt, synthetic construct
  source 1..21: mol_type = other RNA
  modified_base order(1,3,4,6,7,9..13,15,16,18..21): mod_base = OTHER; note = 2-prime-O-methyl RNA nucleotides
  modified_base order(2,5,8,14,17): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
tgaagcgaagtgcacacggtc

SEQ ID NO: 87 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
ccgtgtgcacttcgcttca

SEQ ID NO: 88 - RNA, 21 nt, synthetic construct
  source 1..21: mol_type = other RNA
  modified_base 11: mod_base = OTHER; note = apU nucleotide
  modified_base order(1,3,4,6,7,9,10,12,13,15,16,18..21): mod_base = OTHER; note = 2-prime-O-methyl RNA nucleotides
  modified_base order(2,5,8,14,17): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
tgaagcgaagtgcacacggtc

SEQ ID NO: 89 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
ccgtgtgcacttcgcttca

SEQ ID NO: 90 - RNA, 21 nt, synthetic construct
  source 1..21: mol_type = other RNA
  modified_base order(1,3,4,6,7,9..13,15,16,18,19,21): mod_base = OTHER; note = 2-prime-O-methyl RNA nucleotides
  modified_base order(2,5,8,14,17): mod_base = OTHER; note = 2-prime-fluoro RNA nucleotides
  modified_base 20: mod_base = OTHER; note = apU nucleotide
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
tgaagcgaagtgcacacggtc

SEQ ID NO: 91 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
ccgtgtgcacttcgcttca

SEQ ID NO: 92 - RNA, 21 nt, synthetic construct
  source 1..21: mol_type = other RNA
  modified_base order(3,4,6,7,9..13,15,16,18..21): mod_base = OTHER; note = 2-prime-O-methyl RNA nucleotides
  modified_base order(2,5,8,14,17): mod_base = OTHER; note = 2-prime-fluoro RNA nucleotides
  modified_base 1: mod_base = OTHER; note = apU nucleotide
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
tgaagcgaagtgcacacggtc

SEQ ID NO: 93 - RNA, 19 nt, synthetic construct
  source 1..19: mol_type = other RNA
  modified_base order(1,2,4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 3: mod_base = OTHER; note = 3-prime,4-prime seco modified nucleotide
ccgtgtgcacttcgcttca

SEQ ID NO: 94 - RNA, 22 nt, synthetic construct
  source 1..22: mol_type = other RNA
  modified_base order(3,4,6,7,9..13,15,16,18..21): mod_base = OTHER; note = 2-prime-O-methyl RNA nucleotides
  modified_base 1: mod_base = OTHER; note = Nucleotide Phosphate mimic
  modified_base order(2,5,8,14,17): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
ttgaagcgaagtgcacacggtc

SEQ ID NO: 95 - RNA, 19 nt, synthetic construct
  source 1..19: mol_type = other RNA
  modified_base order(1..3,6,10..19): mod_base = OTHER; note = 2-prime-O-methyl RNA nucleotides
  modified_base order(5,7..9): mod_base = OTHER; note = 2-prime-fluoro RNA nucleotides
  modified_base 4: mod_base = OTHER; note = 3-prime,4-prime seco modified nucleotide
  modified_base 1^2: mod_base = OTHER; note = phosphorothioate
  modified_base 2^3: mod_base = OTHER; note = phosphorothioate
ccgtgtgcacttcgcttca

SEQ ID NO: 96 - RNA, 19 nt, synthetic construct
  source 1..19: mol_type = other RNA
  modified_base order(1..4,10..19): mod_base = OTHER; note = 2-prime-O-methyl RNA nucleotides
  modified_base order(5,7..9): mod_base = OTHER; note = 2-prime-fluoro RNA nucleotides
  modified_base 6: mod_base = OTHER; note = 3-prime,4-prime seco modified nucleotide
  modified_base 1^2: mod_base = OTHER; note = phosphorothioate
  modified_base 2^3: mod_base = OTHER; note = phosphorothioate
ccgtgtgcacttcgcttca

SEQ ID NO: 97 - RNA, 19 nt, synthetic construct
  source 1..19: mol_type = other RNA
  modified_base order(1..4,6,11..19): mod_base = OTHER; note = 2-prime-O-methyl RNA nucleotides
  modified_base order(5,7..9): mod_base = OTHER; note = 2-prime-fluoro RNA nucleotides
  modified_base 10: mod_base = OTHER; note = 3-prime,4-prime seco modified nucleotide
  modified_base 1^2: mod_base = OTHER; note = phosphorothioate
  modified_base 2^3: mod_base = OTHER; note = phosphorothioate
ccgtgtgcacttcgcttca

SEQ ID NO: 98 - RNA, 19 nt, synthetic construct
  source 1..19: mol_type = other RNA
  modified_base order(1..4,6,10,12..19): mod_base = OTHER; note = 2-prime-O-methyl RNA nucleotides
  modified_base order(5,7..9): mod_base = OTHER; note = 2-prime-fluoro RNA nucleotides
  modified_base 11: mod_base = OTHER; note = 3-prime,4-prime seco modified nucleotide
  modified_base 1^2: mod_base = OTHER; note = phosphorothioate
  modified_base 2^3: mod_base = OTHER; note = phosphorothioate
ccgtgtgcacttcgcttca

SEQ ID NO: 99 - RNA, 19 nt, synthetic construct
  source 1..19: mol_type = other RNA
  modified_base order(1..4,6,10,11,13..19): mod_base = OTHER; note = 2-prime-O-methyl RNA nucleotides
  modified_base order(5,7..9): mod_base = OTHER; note = 2-prime-fluoro RNA nucleotides
  modified_base 12: mod_base = OTHER; note = 3-prime,4-prime seco modified nucleotide
  modified_base 1^2: mod_base = OTHER; note = phosphorothioate
  modified_base 2^3: mod_base = OTHER; note = phosphorothioate
ccgtgtgcacttcgcttca

SEQ ID NO: 100 - RNA, 19 nt, synthetic construct
  source 1..19: mol_type = other RNA
  modified_base order(1..4,6,10..12,14..19): mod_base = OTHER; note = 2-prime-O-methyl RNA nucleotides
  modified_base order(5,7..9): mod_base = OTHER; note = 2-prime-fluoro RNA nucleotides
  modified_base 13: mod_base = OTHER; note = 3-prime,4-prime seco modified nucleotide
  modified_base 1^2: mod_base = OTHER; note = phosphorothioate
  modified_base 2^3: mod_base = OTHER; note = phosphorothioate
ccgtgtgcacttcgcttca

SEQ ID NO: 101 - RNA, 19 nt, synthetic construct
  source 1..19: mol_type = other RNA
  modified_base order(1..4,6,10..13,15..19): mod_base = OTHER; note = 2-prime-O-methyl RNA nucleotides
  modified_base order(5,7..9): mod_base = OTHER; note = 2-prime-fluoro RNA nucleotides
  modified_base 14: mod_base = OTHER; note = 3-prime,4-prime seco modified nucleotide
  modified_base 1^2: mod_base = OTHER; note = phosphorothioate
  modified_base 2^3: mod_base = OTHER; note = phosphorothioate
ccgtgtgcacttcgcttca

SEQ ID NO: 102 - RNA, 19 nt, synthetic construct
  source 1..19: mol_type = other RNA
  modified_base order(1..4,6,10..14,16..19): mod_base = OTHER; note = 2-prime-O-methyl RNA nucleotides
  modified_base order(5,7..9): mod_base = OTHER; note = 2-prime-fluoro RNA nucleotides
  modified_base 15: mod_base = OTHER; note = 3-prime,4-prime seco modified nucleotide
  modified_base 1^2: mod_base = OTHER; note = phosphorothioate
  modified_base 2^3: mod_base = OTHER; note = phosphorothioate
ccgtgtgcacttcgcttca

SEQ ID NO: 103 - RNA, 19 nt, synthetic construct
  source 1..19: mol_type = other RNA
  modified_base order(1..4,6,10..15,17..19): mod_base = OTHER; note = 2-prime-O-methyl RNA nucleotides
  modified_base order(5,7..9): mod_base = OTHER; note = 2-prime-fluoro RNA nucleotides
  modified_base 16: mod_base = OTHER; note = 3-prime,4-prime seco modified nucleotide
  modified_base 1^2: mod_base = OTHER; note = phosphorothioate
  modified_base 2^3: mod_base = OTHER; note = phosphorothioate
ccgtgtgcacttcgcttca

SEQ ID NO: 104 - RNA, 19 nt, synthetic construct
  source 1..19: mol_type = other RNA
  modified_base order(1..4,6,10..16,18,19): mod_base = OTHER; note = 2-prime-O-methyl RNA nucleotides
  modified_base order(5,7..9): mod_base = OTHER; note = 2-prime-fluoro RNA nucleotides
  modified_base 17: mod_base = OTHER; note = 3-prime,4-prime seco modified nucleotide
  modified_base 1^2: mod_base = OTHER; note = phosphorothioate
  modified_base 2^3: mod_base = OTHER; note = phosphorothioate
ccgtgtgcacttcgcttca

SEQ ID NO: 105 - RNA, 19 nt, synthetic construct
  source 1..19: mol_type = other RNA
  modified_base order(1..4,6,10..17,19): mod_base = OTHER; note = 2-prime-O-methyl RNA nucleotides
  modified_base order(5,7..9): mod_base = OTHER; note = 2-prime-fluoro RNA nucleotides
  modified_base 18: mod_base = OTHER; note = 3-prime,4-prime seco modified nucleotide
  modified_base 1^2: mod_base = OTHER; note = phosphorothioate
  modified_base 2^3: mod_base = OTHER; note = phosphorothioate
ccgtgtgcacttcgcttca

SEQ ID NO: 106 - RNA, 19 nt, synthetic construct
  source 1..19: mol_type = other RNA
  modified_base order(1,2,4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 3: mod_base = OTHER; note = 3-prime,4-prime seco modified nucleotide
  modified_base 1^2: mod_base = OTHER; note = phosphorothioate
  modified_base 2^3: mod_base = OTHER; note = phosphorothioate
ccgtgtgcacttcgcttca

SEQ ID NO: 107 - RNA, 21 nt, synthetic construct
  source 1..21: mol_type = other RNA
  modified_base order(1,3,4,6,7,9..13,15,16,18..21): mod_base = OTHER; note = 2-prime-O-methyl RNA nucleotides
  modified_base order(2,5,8,14,17): mod_base = OTHER; note = 2-prime-fluoro RNA nucleotides
  modified_base 1^2: mod_base = OTHER; note = phosphorothioate
  modified_base 18^19: mod_base = OTHER; note = phosphorothioate
  modified_base 19^20: mod_base = OTHER; note = phosphorothioate
tgaagcgaagtgcacacggtc

SEQ ID NO: 108 - RNA, 19 nt, synthetic construct
  source 1..19: mol_type = other RNA
  modified_base order(1..4,6,10..15,17..19): mod_base = OTHER; note = 2-prime-O-methyl RNA nucleotides
  modified_base order(5,7..9): mod_base = OTHER; note = 2-prime-fluoro RNA nucleotides
  modified_base 16: mod_base = OTHER; note = 3-prime,4-prime seco modified nucleotide
  modified_base 1^2: mod_base = OTHER; note = phosphorothioate
  modified_base 2^3: mod_base = OTHER; note = phosphorothioate
ccgtgtgcacttcgcttca

SEQ ID NO: 109 - RNA, 19 nt, synthetic construct
  source 1..19: mol_type = other RNA
  modified_base order(1..4,6,10..16,18,19): mod_base = OTHER; note = 2-prime-O-methyl RNA nucleotides
  modified_base order(5,7..9): mod_base = OTHER; note = 2-prime-fluoro RNA nucleotides
  modified_base 17: mod_base = OTHER; note = 3-prime,4-prime seco modified nucleotide
  modified_base 1^2: mod_base = OTHER; note = phosphorothioate
  modified_base 2^3: mod_base = OTHER; note = phosphorothioate
ccgtgtgcacttcgcttca

SEQ ID NO: 110 - RNA, 19 nt, synthetic construct
  source 1..19: mol_type = other RNA
  modified_base order(1..4,6,10..17,19): mod_base = OTHER; note = 2-prime-O-methyl RNA nucleotides
  modified_base order(5,7..9): mod_base = OTHER; note = 2-prime-fluoro RNA nucleotides
  modified_base 18: mod_base = OTHER; note = 3-prime,4-prime seco modified nucleotide
  modified_base 1^2: mod_base = OTHER; note = phosphorothioate
  modified_base 2^3: mod_base = OTHER; note = phosphorothioate
ccgtgtgcacttcgcttca

SEQ ID NO: 111 - RNA, 19 nt, synthetic construct
  source 1..19: mol_type = other RNA
  modified_base order(1..4,6,10..19): mod_base = OTHER; note = 2-prime-O-methyl RNA nucleotides
  modified_base order(5,7..9): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
ccgtgtgcacttcgcttca

SEQ ID NO: 112 - RNA, 21 nt, synthetic construct
  source 1..21: mol_type = other RNA
  modified_base 1: mod_base = OTHER; note = Nucleotide Phosphate mimic
  modified_base order(3,4,6,7,9..13,15,16,18..21): mod_base = OTHER; note = 2-prime-O-methyl RNA nucleotides
  modified_base order(2,5,8,14,17): mod_base = OTHER; note = 2-prime-fluoro RNA nucleotides
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
tgaagcgaagtgcacacggtc

SEQ ID NO: 113 - DNA, 29903 bp, synthetic construct
  source 1..29903: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 114 - RNA, 21 nt, synthetic construct
  source 1..21: mol_type = other RNA
  modified_base order(1,3,4,6,7,9..13,15,16,18..21): mod_base = OTHER; note = 2-prime-O-methyl RNA nucleotides
  modified_base order(2,5,8,14,17): mod_base = OTHER; note = 2-prime-fluoro RNA nucleotides
  modified_base 1^2: mod_base = OTHER; note = phosphorothioate
  modified_base 18^19: mod_base = OTHER; note = phosphorothioate
  modified_base 19^20: mod_base = OTHER; note = phosphorothioate
tgaagcgaagtgcacacggtc

(19) United States
(12) Patent Application Publication
(10) Pub. No.: US 2026/0002157 A1
(43) Pub. Date: Jan. 1, 2026

(54) MODIFIED SHORT INTERFERING NUCLEIC ACID (siNA) MOLECULES AND USES THEREOF

(71) Applicant: Aligos Therapeutics, Inc., South San Francisco, CA (US)

(72) Inventors: Leonid Beigelman, San Mateo, CA (US); Vivek Kumar Rajwanshi, Cupertino, CA (US); Markus Hossbach, Kulmbach (DE); Rajendra K. Pandey, Foster City, CA (US); Jin Hong, Pacifica, CA (US); Laxman Eltepu, San Ramon, CA (US); Saul Martinez Montero, San Bruno, CA (US); N. Tilani S. De Costa, South San Francisco, CA (US)

(73) Assignee: Aligos Therapeutics, Inc., South San Francisco, CA (US)

(21) Appl. No.: 19/323,023

(22) Filed: Sep. 9, 2025

Related U.S. Application Data
(62) Division of application No. 17/939,896, filed on Sep. 7, 2022, now Pat. No. 12,454,691.
(60) Provisional application No. 63/241,935, filed on Sep. 8, 2021.

(51) Int. Cl. C12N 15/113; A61P 31/20; C07H 19/067; C07H 19/167; C07H 21/02

(52) U.S. Cl. CPC C12N 15/113; A61P 31/20; C07H 19/067; C07H 19/167; C07H 21/02; C12N 2310/11; C12N 2310/14; C12N 2310/314; C12N 2310/315

(57) ABSTRACT

Described are short interfering nucleic acid (siNA) molecules comprising modified nucleotides, compositions, and methods and uses thereof.

### CROSS-REFERENCE TO RELATED APPLICATION

This application is a Divisional of U.S. application Ser. No. 17/939,896, filed Sep. 7, 2022, which claims priority to U.S. Provisional Application No. 63/241,935, filed Sep. 8, 2021, the disclosures of which are hereby incorporated by reference in their entireties.

The instant application contains a Sequence Listing which has been submitted in XML format via Patent Center and is hereby incorporated by reference in its entirety. Said XML copy, created on Nov. 18, 2022, is named 122400-0305.xml and is 471,010 bytes.

### TECHNICAL FIELD

Described are short interfering nucleic acid (siNA) molecules comprising modified nucleotides, compositions, and methods and uses thereof.

### BACKGROUND

RNA interference (RNAi) is a biological response to double-stranded RNA that mediates resistance to both endogenous parasitic and exogenous pathogenic nucleic acids, and regulates the expression of protein-coding genes. The short interfering nucleic acids (siNA), such as siRNA, have been developed for RNAi therapy to treat a variety of diseases. For instance, RNAi therapy has been proposed for the treatment of metabolic diseases, neurodegenerative diseases, cancer, and pathogenic infections (See e.g., Rondindone, Biotechniques, 2018, 40(4S), doi.org/10.2144/000112163, Boudreau and Davidson, Curr Top Dev Biol, 2006, 75:73-92, Chalbatani et al., Int J Nanomedicine, 2019, 14:3111-3128, Arbuthnot, Drug News Perspect, 2010, 23(6):341-50, and Chernikov et. al., Front. Pharmacol., 2019, doi.org/10.3389/fphar.2019.00444, each of which are incorporated by reference in their entirety). However, major limitations of RNAi therapy are the ability to effectively deliver siRNA to target cells and the degradation of the siRNA.

The present disclosure improves the delivery and stability of siNA molecules by providing siNA molecules comprising modified nucleobases. The siNA molecules of the present disclosure provide optimized combinations and numbers of modified nucleotides, nucleotide lengths, design (e.g., blunt ends or overhangs, internucleoside linkages, conjugates), and modification patterns for improving the delivery and stability of siNA molecules.

### SUMMARY

Described herein are short interfering nucleic acid (siNA) molecules comprising novel modified nucleobase monomers, phosphate mimics, and/or other modifications. Also described herein are methods of using the disclosed siNA molecules for treating various diseases and conditions.

In a first aspect, the present disclosure provides a nucleotide comprising a structure of:

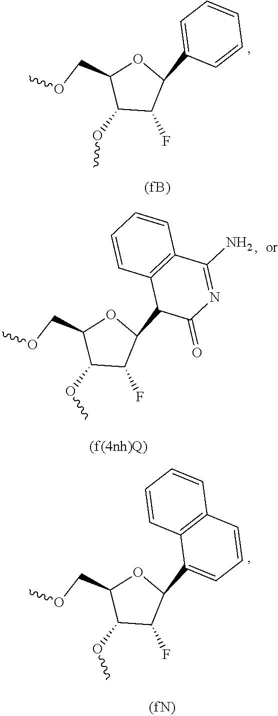

and nucleic acid sequences and siNA comprising any one of the foregoing nucleotides or a combination of nucleotides thereof.

In a second aspect, the present disclosure provides a nucleotide comprising a structure of:

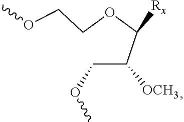

wherein Rx is a nucleobase, aryl, heteroaryl, or H. For example, the nucleotide may comprise a structure of:

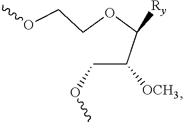

wherein R_{y} is a nucleobase.

In a third aspect, the present disclosure provides a nucleotide comprising a structure of:

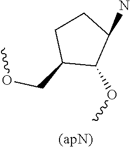

wherein R_{y} is a nucleobase, and nucleic acid sequences and siNA comprising the foregoing nucleotide. In some embodiments, the nucleotide may comprise a structure of:

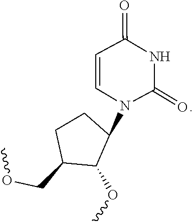

In a fourth aspect, the present disclosure provides a nucleotide phosphate mimic comprising a structure of:

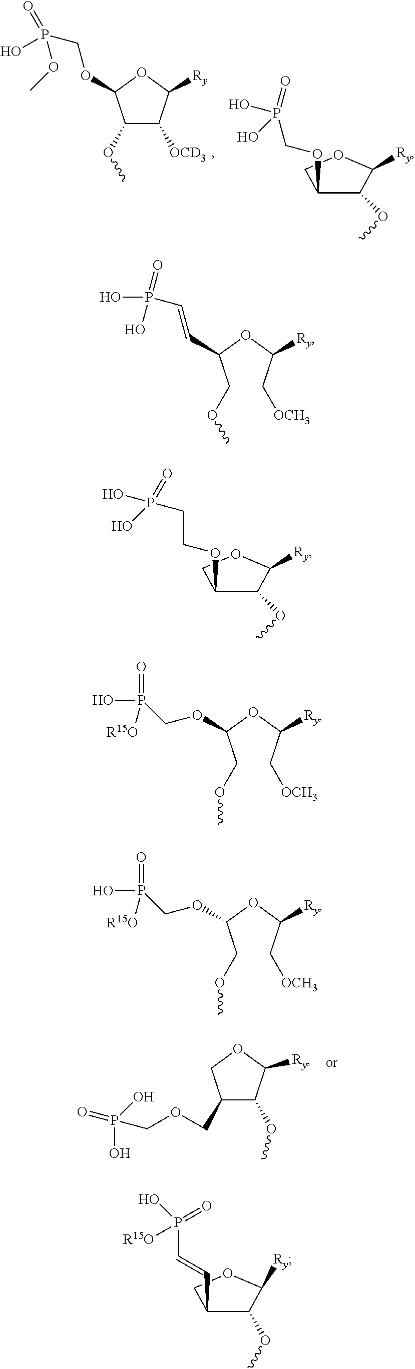

wherein R_{y} is a nucleobase and R¹⁵ is H or CH₃.

The present disclosure provides short interfering nucleic acid (siNA) molecules comprising at least one, at least two, at least 3, at least 4, or at least 5 nucleotide(s) according to the first, second, or third aspects, which optionally may be located in and/or capable of destabilizing a seed region of the siNA. In some embodiments, the antisense strand may comprise a 5′-stabilized end cap selected from:

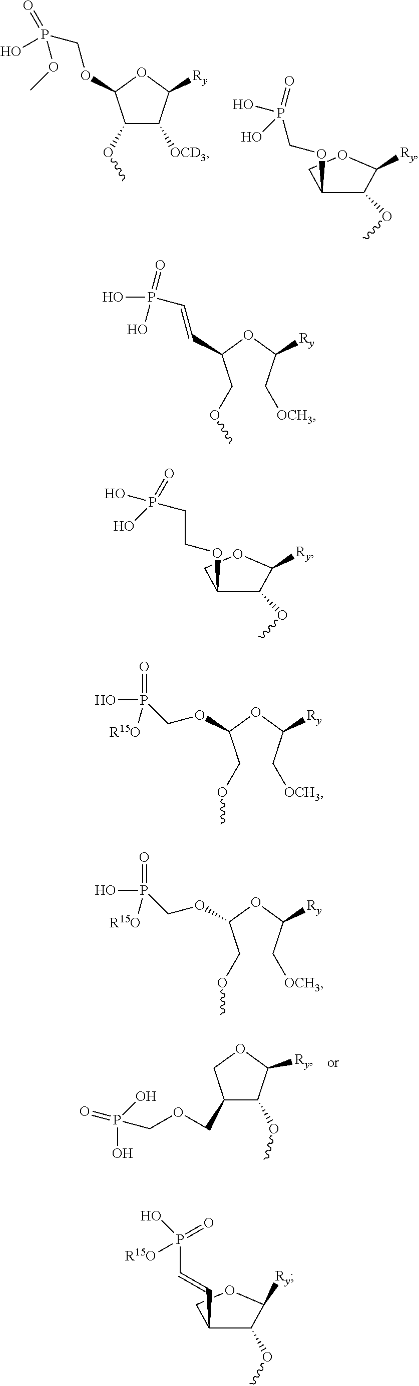

wherein R_{y} is a nucleobase and R¹⁵ is H or CH₃.

The present disclosure provides short interfering nucleic acid (siNA) molecules comprising a sense strand and an antisense strand, wherein the antisense comprises a nucleotide phosphate mimic according to the fourth aspect at its 5′ end.

The present disclosure provides short interfering nucleic acid (siNA) molecules comprising:
(a) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence:
is 15 to 30 nucleotides in length; and
comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 3, 5, 7, 8, 9, 10, 11, 12, 14, 17, and/or 19 from the 5′ end of the first nucleotide sequence is a 2′-fluoro nucleotide or wherein at least one modified nucleotide is a 2′-O-methyl nucleotide and at least one modified nucleotide is a 2′-fluoro nucleotide; and
an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence:
is 15 to 30 nucleotides in length; and
comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; or
(b) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence:
(i) is 15 to 30 nucleotides in length; and
(ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; and an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence:
(iii) is 15 to 30 nucleotides in length; and
(iv) comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 2, 5, 6, 8, 10, 14, 16, 17, and/or 18 from the 5′ end of the second nucleotide sequence is a 2′-fluoro nucleotide;
wherein the sense strand and/or the antisense strand comprise at least one, at least two, at least 3, at least 4, or at least 5 nucleotide(s) according to the first, second, or third aspects. In some embodiments, the antisense strand may comprise a 5′-stabilized end cap selected from:

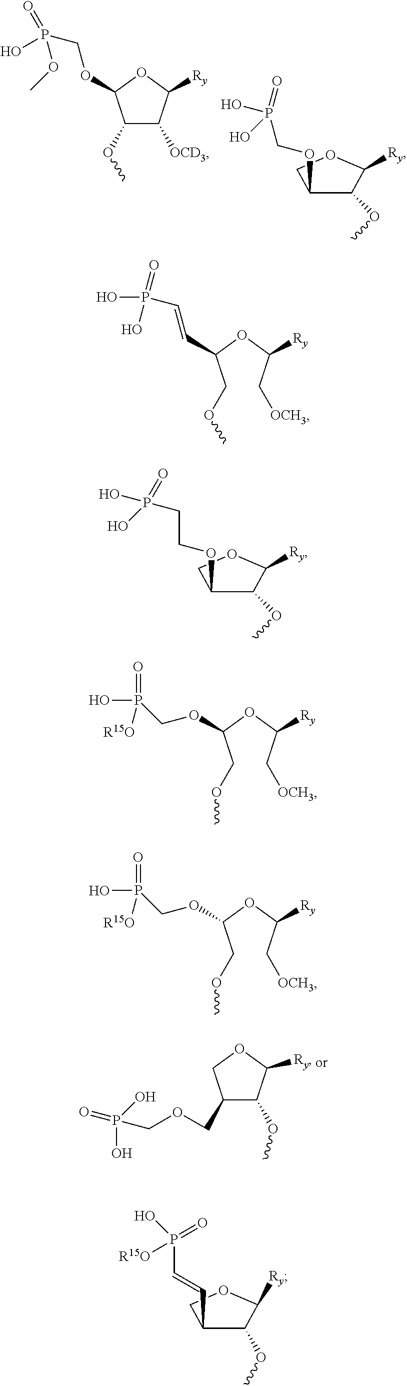

wherein R_{y} is a nucleobase and R¹⁵ is H or CH₃.

The present disclosure provides short interfering nucleic acid (siNA) molecules comprising:
(a) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence:
is 15 to 30 nucleotides in length; and
comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 3, 5, 7, 8, 9, 10, 11, 12, 14, 17, and/or 19 from the 5′ end of the first nucleotide sequence is a 2′-fluoro nucleotide or wherein at least one modified nucleotide is a 2′-O-methyl nucleotide and at least one modified nucleotide is a 2′-fluoro nucleotide; and
an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence:
is 15 to 30 nucleotides in length; and
comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; or
(b) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence:
(i) is 15 to 30 nucleotides in length; and
(ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; and an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence:
(iii) is 15 to 30 nucleotides in length; and
(iv) comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 2, 5, 6, 8, 10, 14, 16, 17, and/or 18 from the 5′ end of the second nucleotide sequence is a 2′-fluoro nucleotide;
wherein the antisense strand comprises a nucleotide phosphate mimic according the fourth aspect at its 5′ end.

In some embodiments of the disclosed siNA molecules, the sense strand and/or the antisense strand independently comprise 1 or more phosphorothioate internucleoside linkages.

In some embodiments of the disclosed siNA molecules, the sense strand and/or the antisense strand independently comprise 1 or more mesyl phosphoroamidate internucleoside linkages.

In some embodiments of the disclosed siNA molecules, the siNA further comprises a phosphorylation blocker, a galactosamine, and/or a 5′-stabilized end cap.

In some embodiments of the disclosed siNA molecules, the sense strand comprises at least 1, 2, 3, 4, 5, 6, 7, 8, 9, 10, 11, 12, 13, 14, 15 or more phosphorothioate internucleoside linkages. In some embodiments, (i) at least one phosphorothioate internucleoside linkage in the sense strand is between the nucleotides at positions 1 and 2 from the 5′ end of the first nucleotide sequence; (ii) at least one phosphorothioate internucleoside linkage is between the nucleotides at positions 2 and 3 from the 5′ end of the first nucleotide sequence.

In some embodiments of the disclosed siNA molecules, the antisense strand further comprises at least 1, 2, 3, 4, 5, 6, 7, 8, 9, 10, 11, 12, 13, 14, 15 or more phosphorothioate internucleoside linkages. In some embodiments, (i) at least one phosphorothioate internucleoside linkage in the antisense strand is between the nucleotides at positions 1 and 2 from the 5′ end of the second nucleotide sequence; (ii) at least one phosphorothioate internucleoside linkage in the antisense strand is between the nucleotides at positions 2 and 3 from the 5′ end of the second nucleotide sequence; (iii) at least one phosphorothioate internucleoside linkage in the antisense strand is between the nucleotides at positions 1 and 2 from the 3′ end of the second nucleotide sequence; and/or (iv) at least one phosphorothioate internucleoside linkage is between the nucleotides at positions 2 and 3 from the 3′ end of the second nucleotide sequence.

In some embodiments of the disclosed siNA molecules, the sense strand comprises at least 1, 2, 3, 4, 5, 6, 7, 8, 9, 10, 11, 12, 13, 14, 15 or more mesyl phosphoroamidate internucleoside linkages. In some embodiments, (i) at least one mesyl phosphoroamidate internucleoside linkage in the sense strand is between the nucleotides at positions 1 and 2 from the 5′ end of the first nucleotide sequence; (ii) at least one mesyl phosphoroamidate internucleoside linkage is between the nucleotides at positions 2 and 3 from the 5′ end of the first nucleotide sequence.

In some embodiments of the disclosed siNA molecules, the antisense strand further comprises at least 1, 2, 3, 4, 5, 6, 7, 8, 9, 10, 11, 12, 13, 14, 15 or more mesyl phosphoroamidate internucleoside linkages. In some embodiments, (i) at least one mesyl phosphoroamidate internucleoside linkage in the antisense strand is between the nucleotides at positions 1 and 2 from the 5′ end of the second nucleotide sequence; (ii) at least one mesyl phosphoroamidate internucleoside linkage in the antisense strand is between the nucleotides at positions 2 and 3 from the 5′ end of the second nucleotide sequence; (iii) at least one mesyl phosphoroamidate internucleoside linkage in the antisense strand is between the nucleotides at positions 1 and 2 from the 3′ end of the second nucleotide sequence; and/or (iv) at least one mesyl phosphoroamidate internucleoside linkage is between the nucleotides at positions 2 and 3 from the 3′ end of the second nucleotide sequence.

The present disclosure additionally provides short interfering nucleic acids (siNAs) comprising a sense strand and an antisense strand, wherein the sense strand and/or the antisense strand independently comprise at least 1, 2, 3, 4, 5, 6, 7, 8, 9, 10, 11, 12, 13, 14, 15 or more mesyl phosphoroamidate internucleoside linkages.

In some embodiments of the disclosed siNA molecules, the antisense strand comprises a 5′-stabilized end cap selected from the group consisting of Formula (1) to Formula (16), Formula (9X) to Formula (12X), Formula (16X), Formula (9Y) to Formula (12Y), Formula (16Y), Formula (21) to Formula (36), Formula 36X, Formula (41) to (56), Formula (49X) to (52X), Formula (49Y) to (52Y), Formula 56X, Formula 56Y, Formula (61), Formula (62), and Formula (63):

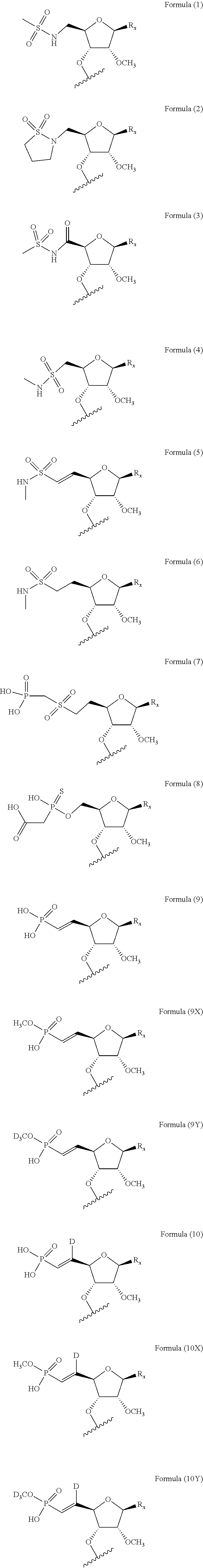

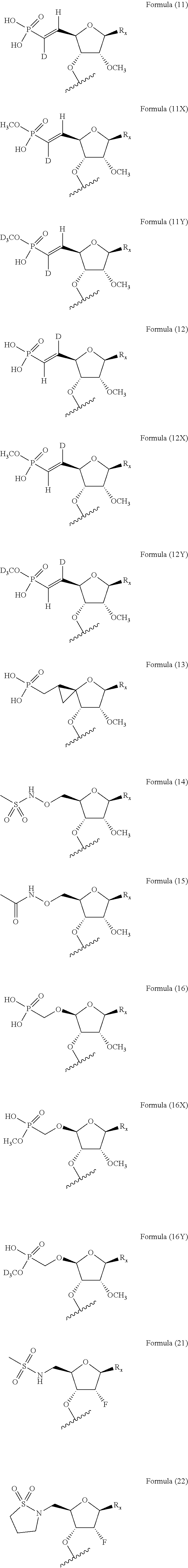

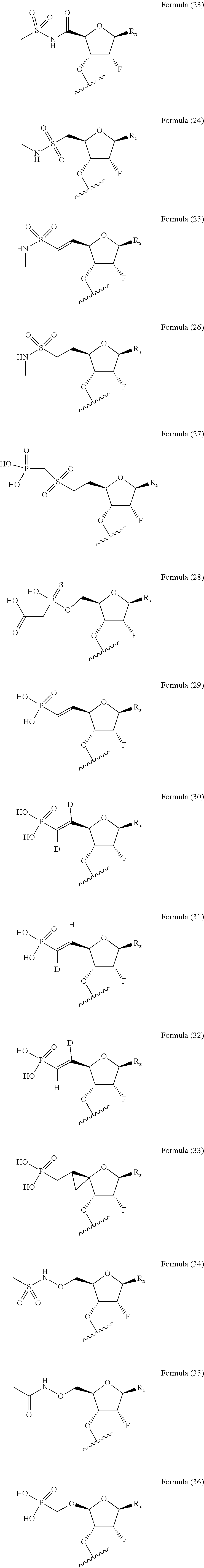

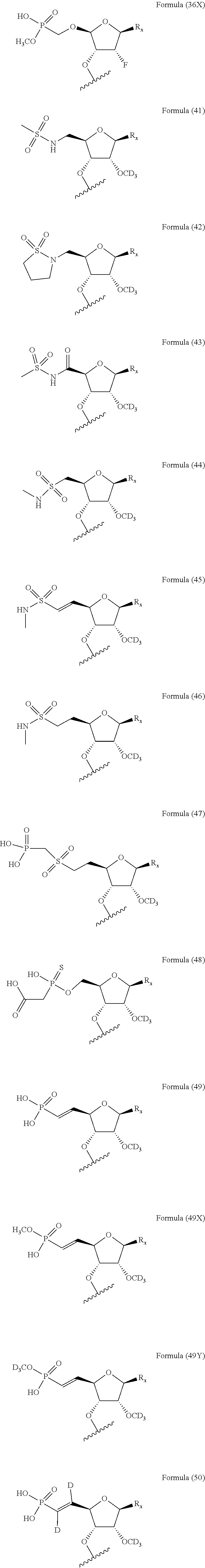

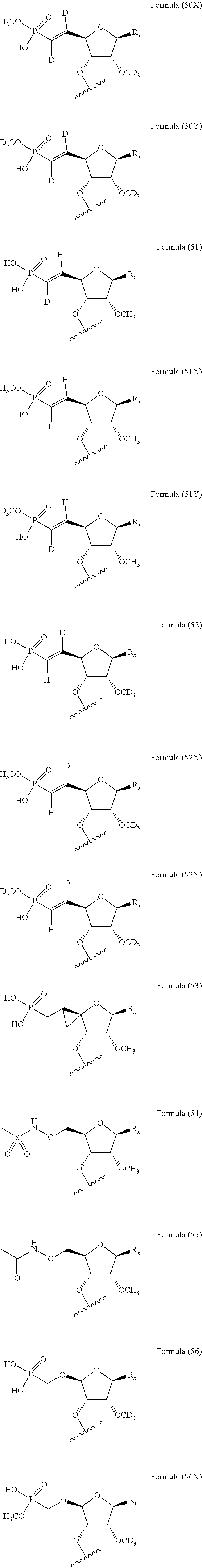

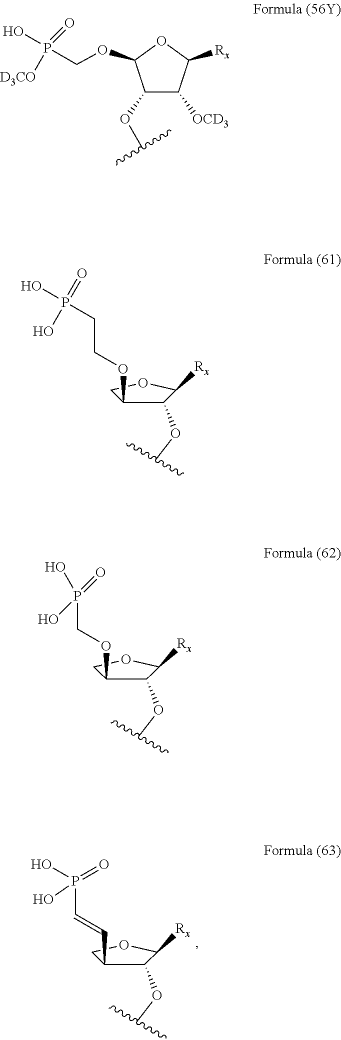

wherein Rₓ is a nucleobase, aryl, heteroaryl, or H.

In some embodiments of the disclosed siNA molecules, the antisense strand comprises a 5′-stabilized end cap selected from the group consisting of Formula (71) to Formula (86), Formula (79X) to Formula (82X), Formula (79Y) to (82Y), Formula 86X, Formula 86X′, Formula 86Y, and Formula 86Y′

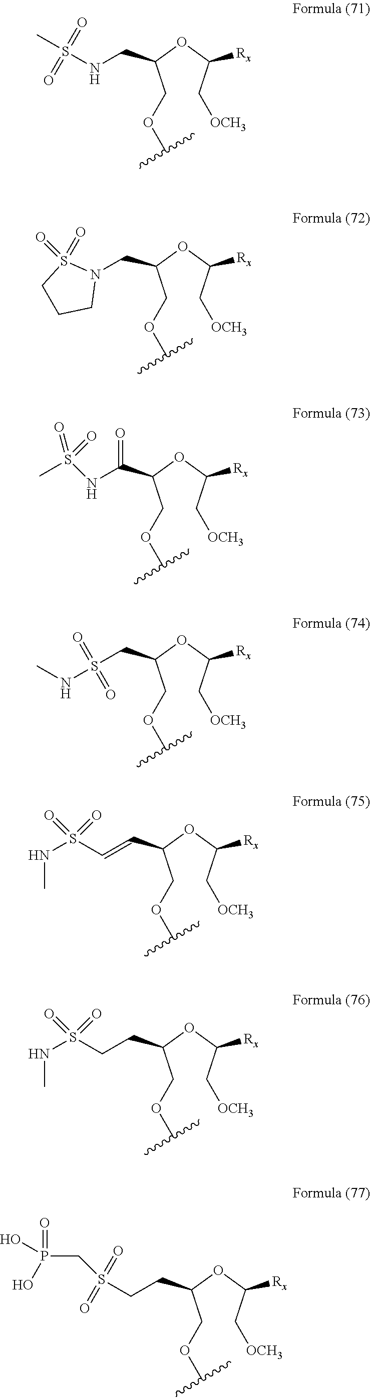

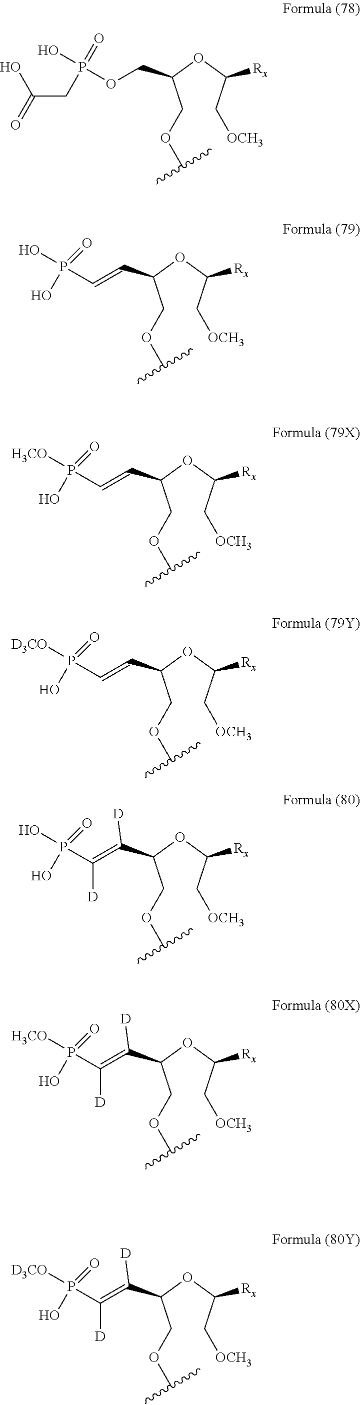

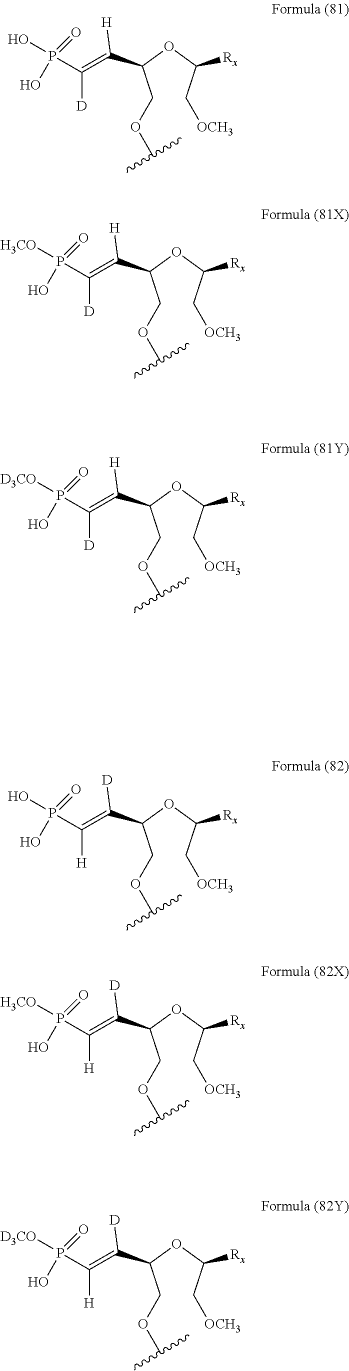

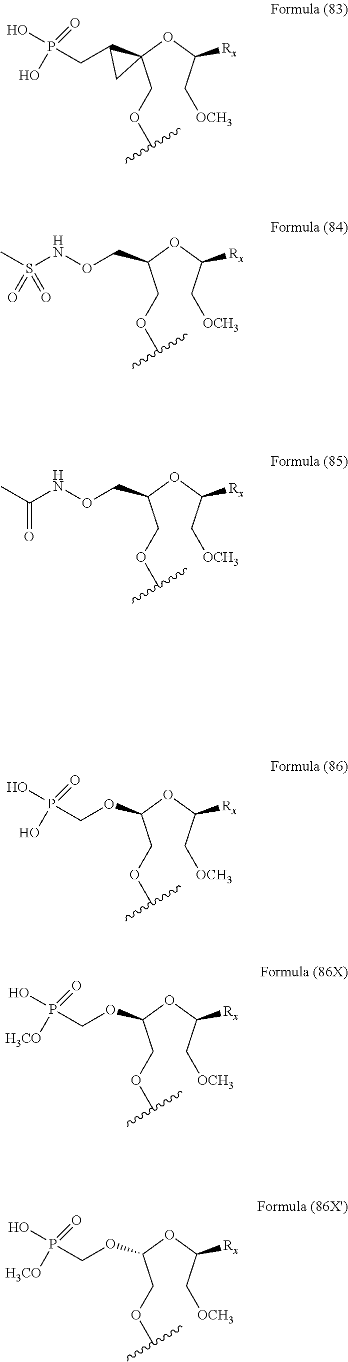

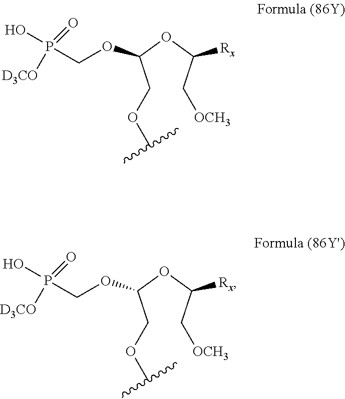

wherein Rₓ is a nucleobase, aryl, heteroaryl, or H.

In some embodiments of the disclosed siNA molecules, the antisense strand comprises a 5′-stabilized end cap selected from the group consisting of Formulas (1A)-(15A), Formulas (1A-1)-(7A-1), Formulas (1A-2)-(7A-2), Formulas (1A-3)-(7A-3), Formulas (1A-4)-(7A-4), Formulas (9B)-(12B), Formulas (9AX)-(12AX), Formulas (9AY)-(12AY), Formulas (9BX)-(12BX), and Formulas (9BY)-(12BY):

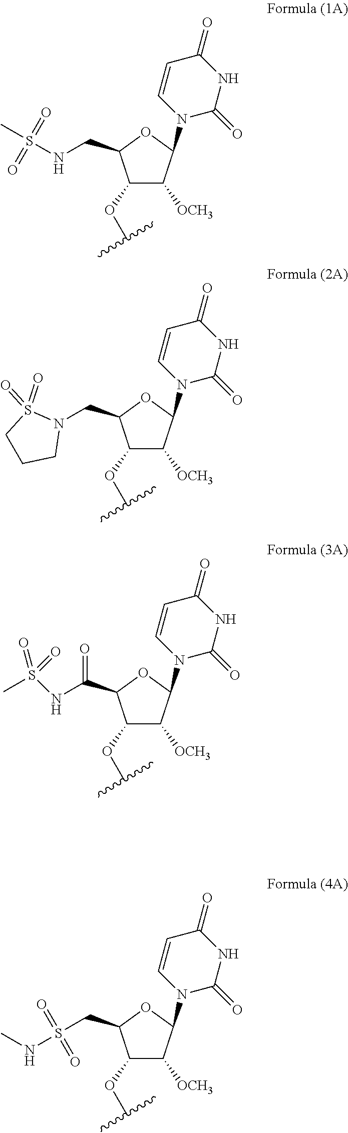 [SMILES: *O[C@H]1[C@@H](OC)[C@H](n2ccc(=O)[nH]c2=O)O[C@@H]1CNS(C)(=O)=O ; *O[C@H]1[C@@H](OC)[C@H](n2ccc(=O)[nH]c2=O)O[C@@H]1CN1CCCS1(=O)=O ; *O[C@H]1[C@@H](OC)[C@H](n2ccc(=O)[nH]c2=O)O[C@@H]1C(=O)NS(C)(=O)=O ; *O[C@H]1[C@@H](OC)[C@H](n2ccc(=O)[nH]c2=O)O[C@@H]1CS(=O)(=O)NC]

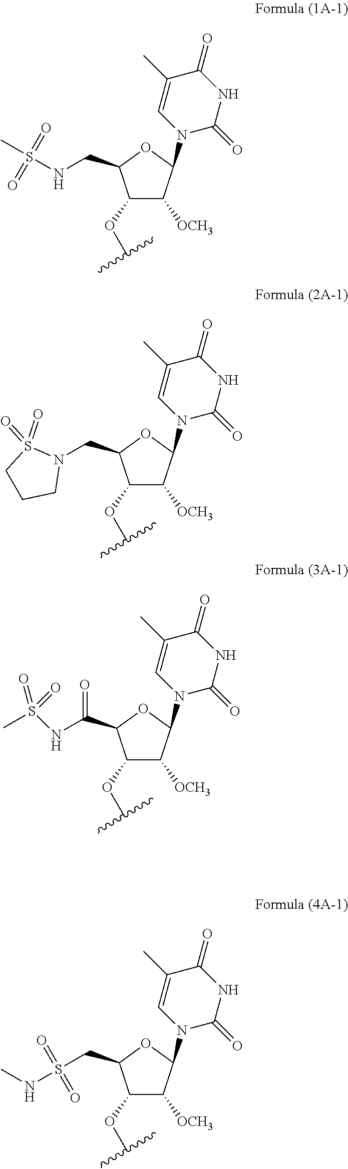 [SMILES: *O[C@H]1[C@@H](OC)[C@H](n2cc(C)c(=O)[nH]c2=O)O[C@@H]1CNS(C)(=O)=O ; *O[C@H]1[C@@H](OC)[C@H](n2cc(C)c(=O)[nH]c2=O)O[C@@H]1CN1CCCS1(=O)=O ; *O[C@H]1[C@@H](OC)[C@H](n2cc(C)c(=O)[nH]c2=O)O[C@@H]1C(=O)NS(C)(=O)=O ; *O[C@H]1[C@@H](OC)[C@H](n2cc(C)c(=O)[nH]c2=O)O[C@@H]1CS(=O)(=O)NC]

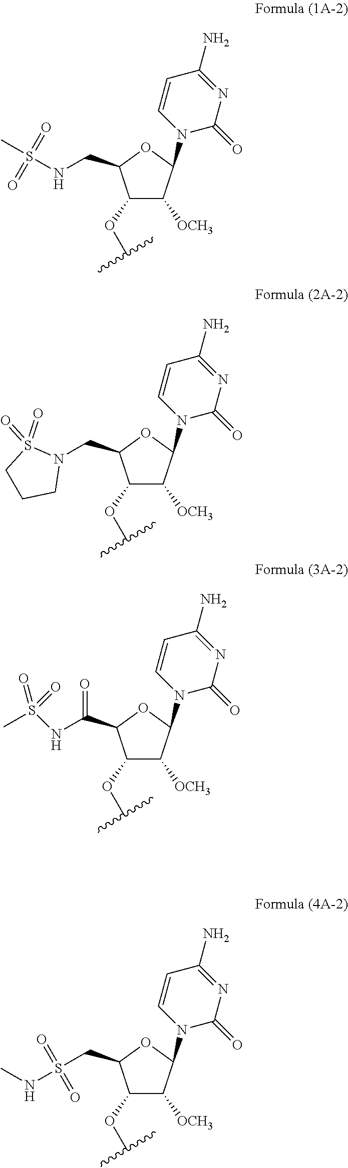 [SMILES: *O[C@H]1[C@@H](OC)[C@H](n2ccc(N)nc2=O)O[C@@H]1CNS(C)(=O)=O ; *O[C@H]1[C@@H](OC)[C@H](n2ccc(N)nc2=O)O[C@@H]1CN1CCCS1(=O)=O ; *O[C@H]1[C@@H](OC)[C@H](n2ccc(N)nc2=O)O[C@@H]1C(=O)NS(C)(=O)=O ; *O[C@H]1[C@@H](OC)[C@H](n2ccc(N)nc2=O)O[C@@H]1CS(=O)(=O)NC]

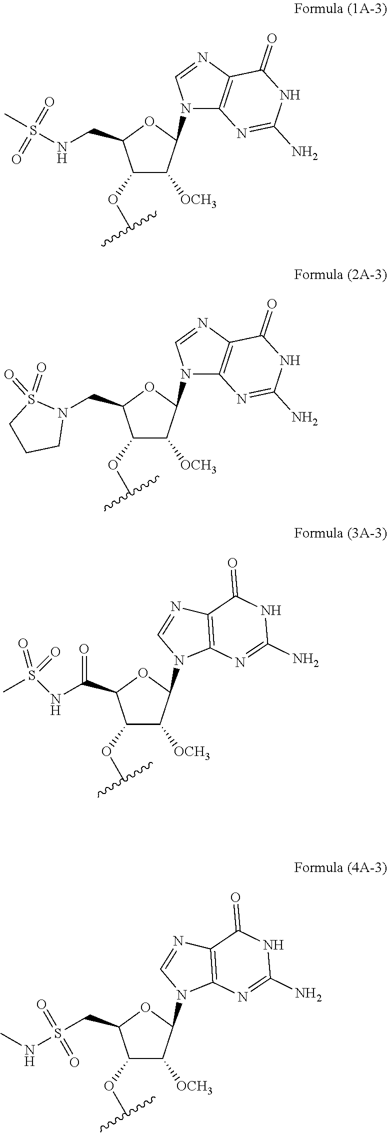 [SMILES: *O[C@H]1[C@@H](OC)[C@H](n2cnc3c(=O)[nH]c(N)nc32)O[C@@H]1CNS(C)(=O)=O ; *O[C@H]1[C@@H](OC)[C@H](n2cnc3c(=O)[nH]c(N)nc32)O[C@@H]1CN1CCCS1(=O)=O ; *O[C@H]1[C@@H](OC)[C@H](n2cnc3c(=O)[nH]c(N)nc32)O[C@@H]1C(=O)NS(C)(=O)=O ; *O[C@H]1[C@@H](OC)[C@H](n2cnc3c(=O)[nH]c(N)nc32)O[C@@H]1CS(=O)(=O)NC]

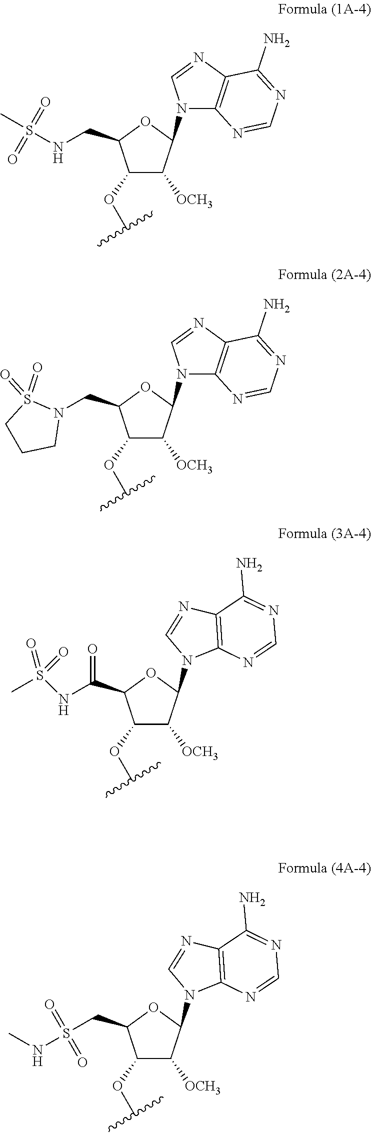 [SMILES: *O[C@H]1[C@@H](OC)[C@H](n2cnc3c(N)ncnc32)O[C@@H]1CNS(C)(=O)=O ; *O[C@H]1[C@@H](OC)[C@H](n2cnc3c(N)ncnc32)O[C@@H]1CN1CCCS1(=O)=O ; *O[C@H]1[C@@H](OC)[C@H](n2cnc3c(N)ncnc32)O[C@@H]1C(=O)NS(C)(=O)=O ; *O[C@H]1[C@@H](OC)[C@H](n2cnc3c(N)ncnc32)O[C@@H]1CS(=O)(=O)NC]

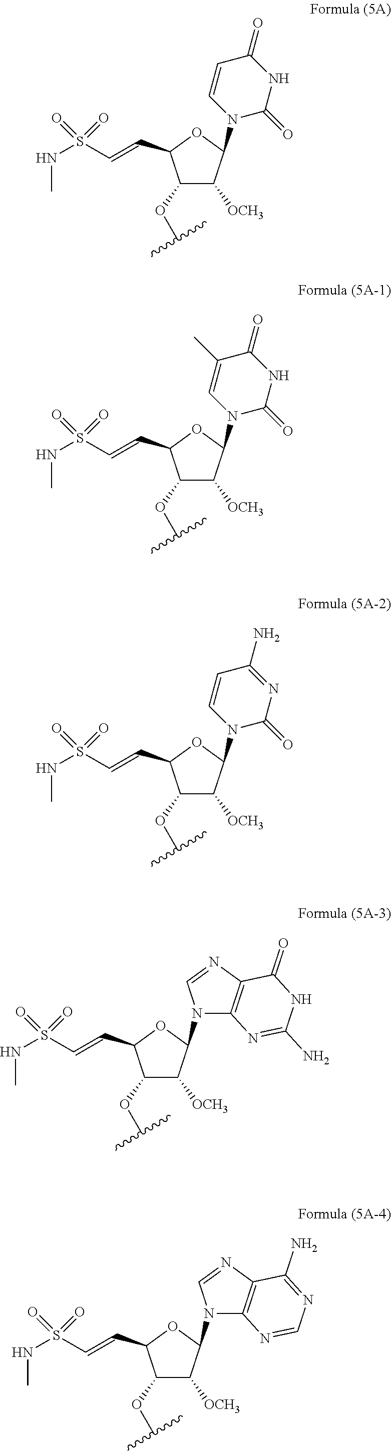 [SMILES: *O[C@H]1[C@@H](OC)[C@H](n2ccc(=O)[nH]c2=O)O[C@@H]1/C=C/S(=O)(=O)NC ; *O[C@H]1[C@@H](OC)[C@H](n2cc(C)c(=O)[nH]c2=O)O[C@@H]1/C=C/S(=O)(=O)NC ; *O[C@H]1[C@@H](OC)[C@H](n2ccc(N)nc2=O)O[C@@H]1/C=C/S(=O)(=O)NC ; *O[C@H]1[C@@H](OC)[C@H](n2cnc3c(=O)[nH]c(N)nc32)O[C@@H]1/C=C/S(=O)(=O)NC ; *O[C@H]1[C@@H](OC)[C@H](n2cnc3c(N)ncnc32)O[C@@H]1/C=C/S(=O)(=O)NC]

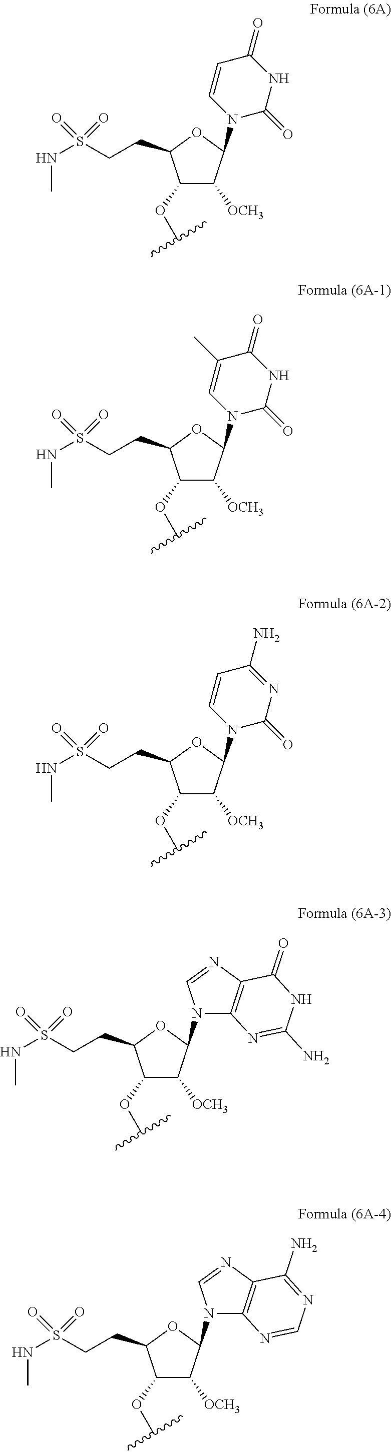 [SMILES: *O[C@H]1[C@@H](OC)[C@H](n2ccc(=O)[nH]c2=O)O[C@@H]1CCS(=O)(=O)NC ; *O[C@H]1[C@@H](OC)[C@H](n2cc(C)c(=O)[nH]c2=O)O[C@@H]1CCS(=O)(=O)NC ; *O[C@H]1[C@@H](OC)[C@H](n2ccc(N)nc2=O)O[C@@H]1CCS(=O)(=O)NC ; *O[C@H]1[C@@H](OC)[C@H](n2cnc3c(=O)[nH]c(N)nc32)O[C@@H]1CCS(=O)(=O)NC ; *O[C@H]1[C@@H](OC)[C@H](n2cnc3c(N)ncnc32)O[C@@H]1CCS(=O)(=O)NC]

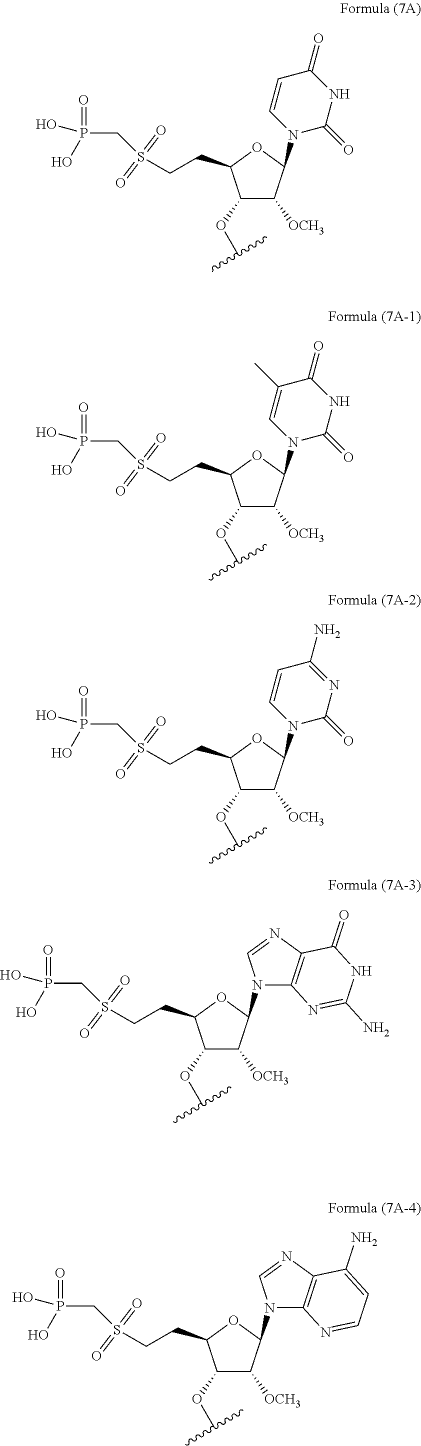 [SMILES: *O[C@H]1[C@@H](OC)[C@H](n2ccc(=O)[nH]c2=O)O[C@@H]1CCS(=O)(=O)CP(=O)(O)O ; *O[C@H]1[C@@H](OC)[C@H](n2cc(C)c(=O)[nH]c2=O)O[C@@H]1CCS(=O)(=O)CP(=O)(O)O ; *O[C@H]1[C@@H](OC)[C@H](n2ccc(N)nc2=O)O[C@@H]1CCS(=O)(=O)CP(=O)(O)O ; *O[C@H]1[C@@H](OC)[C@H](n2cnc3c(=O)[nH]c(N)nc32)O[C@@H]1CCS(=O)(=O)CP(=O)(O)O ; *O[C@H]1[C@@H](OC)[C@H](n2cnc3c(N)ccnc32)O[C@@H]1CCS(=O)(=O)CP(=O)(O)O]

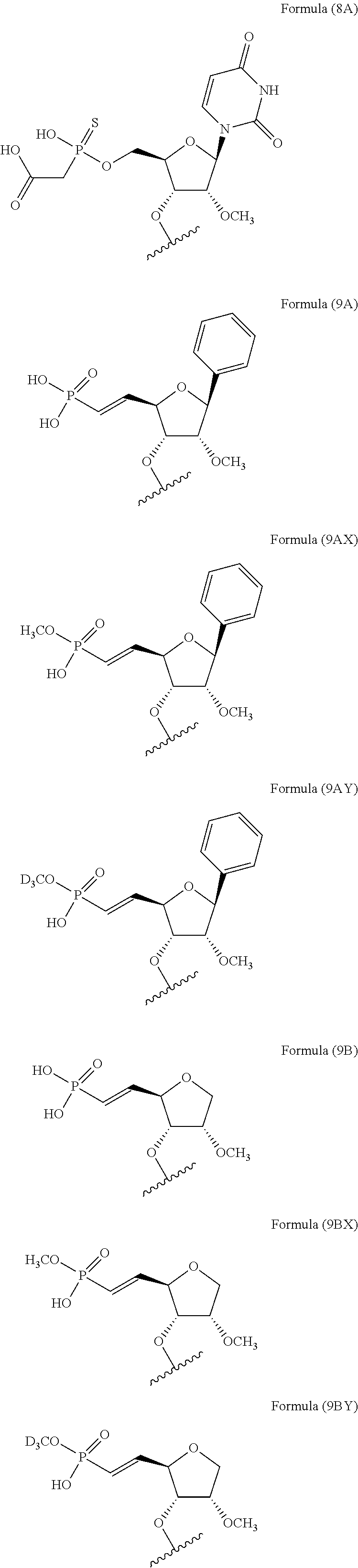

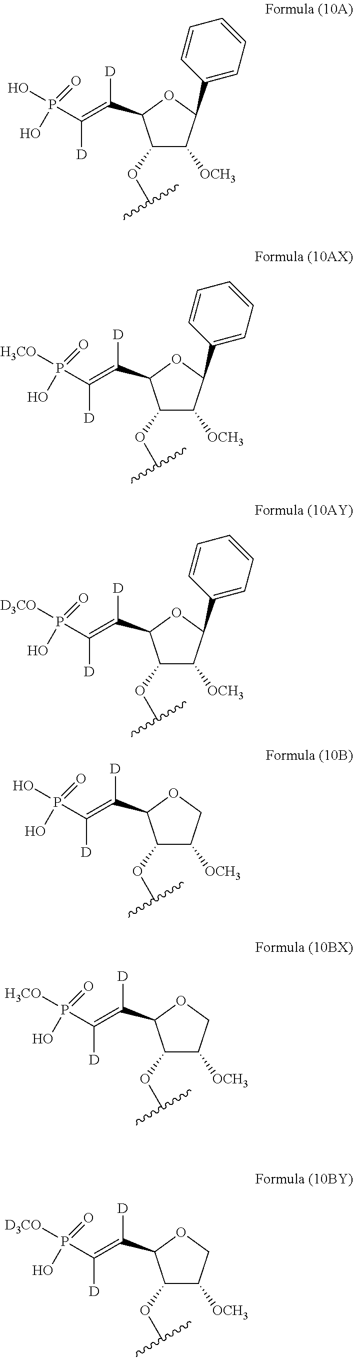

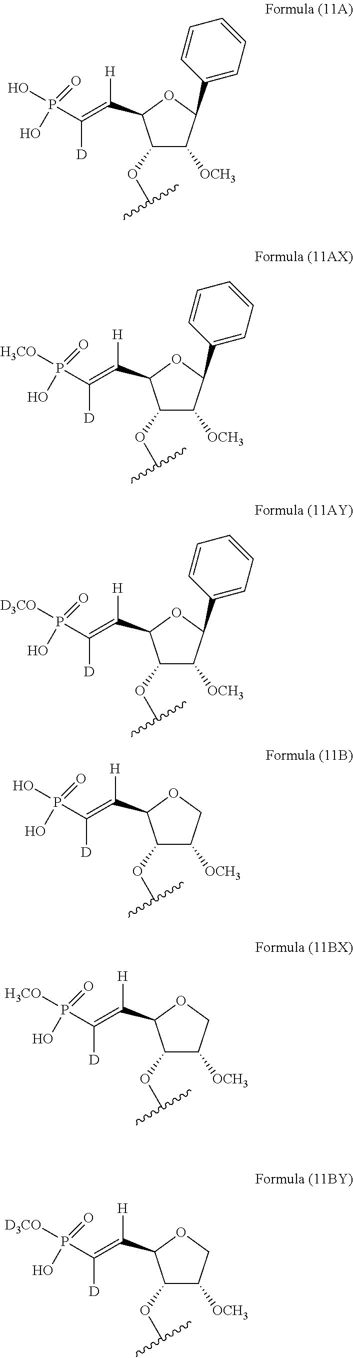

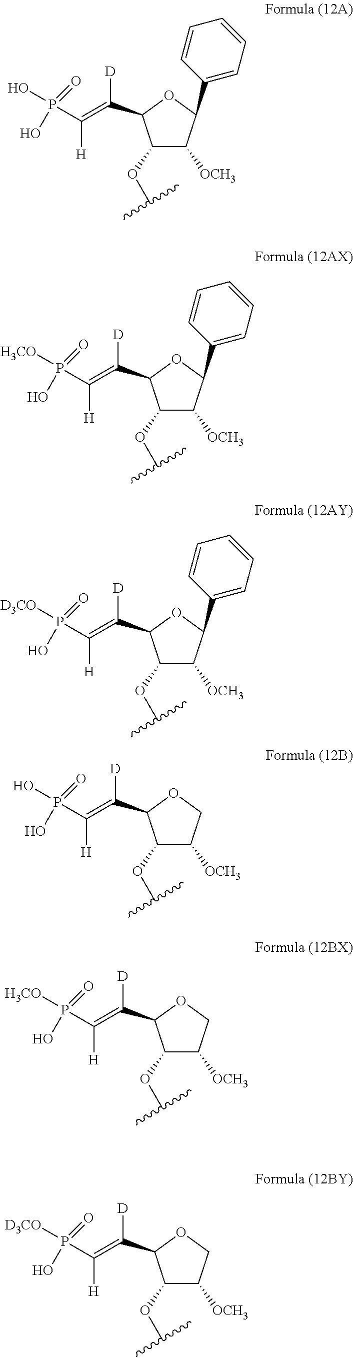

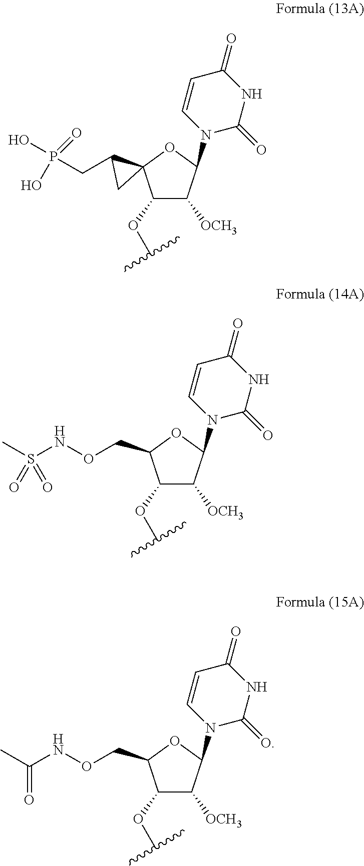

In some embodiments of the disclosed siNA molecules, the antisense strand comprises a 5′-stabilized end cap selected from the group consisting of Formulas (21A)-(35A), Formulas (29B)-(32B), Formulas (29AX)-(32AX), Formulas (29AY)-(32AY), Formulas (29BX)-(32BX), and Formulas (29BY)-(32BY):

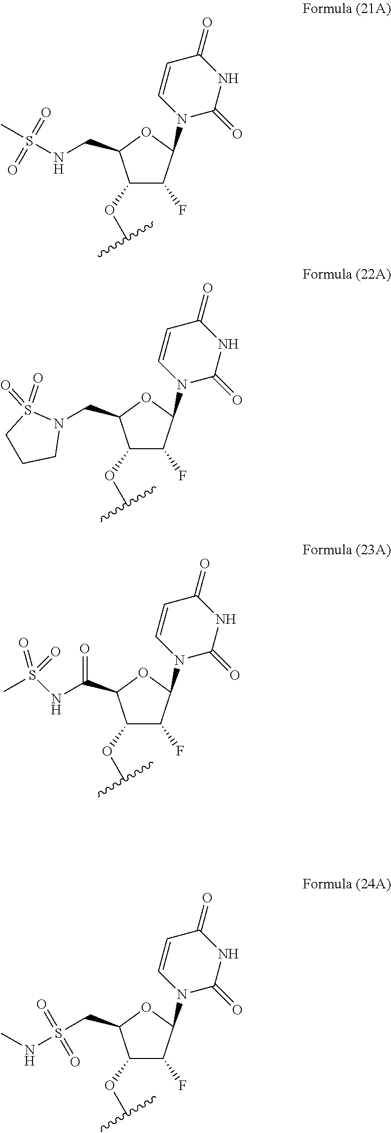 [SMILES: *O[C@H]1[C@@H](F)[C@H](n2ccc(=O)[nH]c2=O)O[C@@H]1CNS(C)(=O)=O ; *O[C@H]1[C@@H](F)[C@H](n2ccc(=O)[nH]c2=O)O[C@@H]1CN1CCCS1(=O)=O ; *O[C@H]1[C@@H](F)[C@H](n2ccc(=O)[nH]c2=O)O[C@@H]1C(=O)NS(C)(=O)=O ; *O[C@H]1[C@@H](F)[C@H](n2ccc(=O)[nH]c2=O)O[C@@H]1CS(=O)(=O)NC]

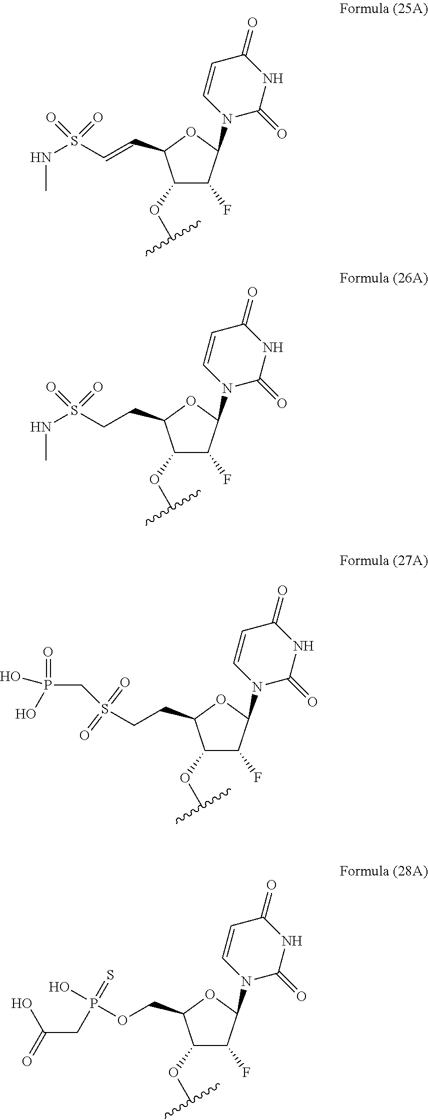 [SMILES: *O[C@H]1[C@@H](F)[C@H](n2ccc(=O)[nH]c2=O)O[C@@H]1/C=C/S(=O)(=O)NC ; *O[C@H]1[C@@H](F)[C@H](n2ccc(=O)[nH]c2=O)O[C@@H]1CCS(=O)(=O)NC ; *O[C@H]1[C@@H](F)[C@H](n2ccc(=O)[nH]c2=O)O[C@@H]1CCS(=O)(=O)CP(=O)(O)O ; *O[C@H]1[C@@H](F)[C@H](n2ccc(=O)[nH]c2=O)O[C@@H]1COP(O)(=S)CC(=O)O]

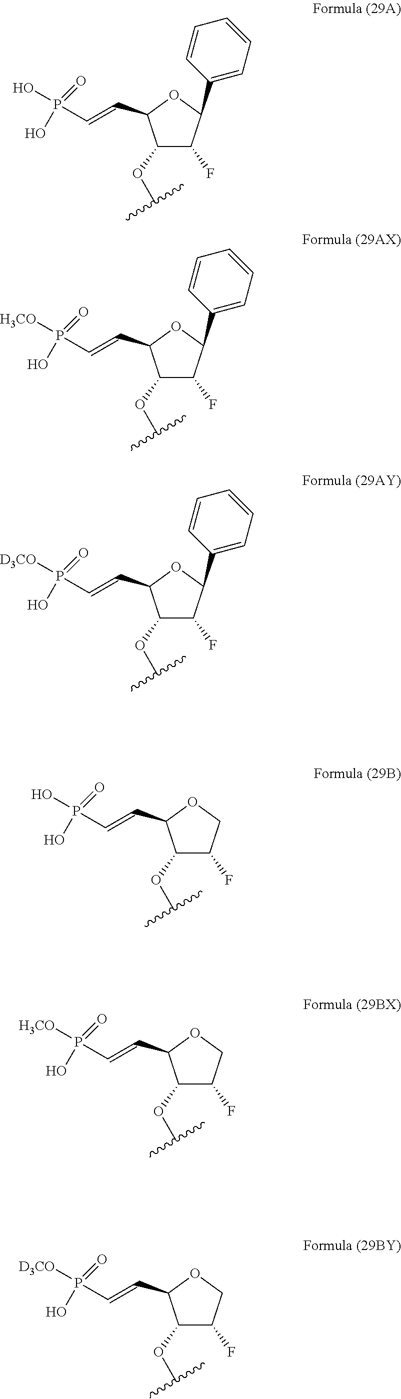

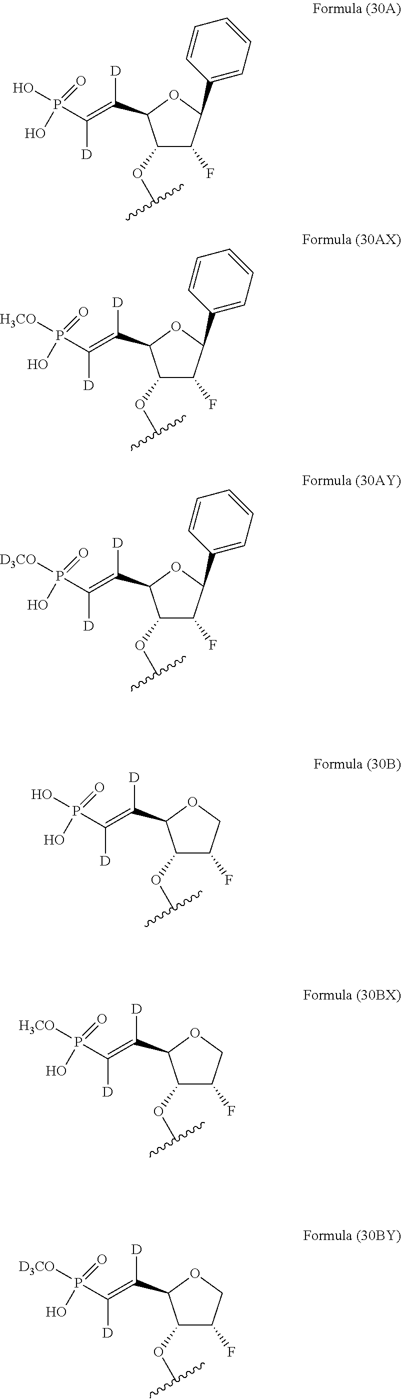

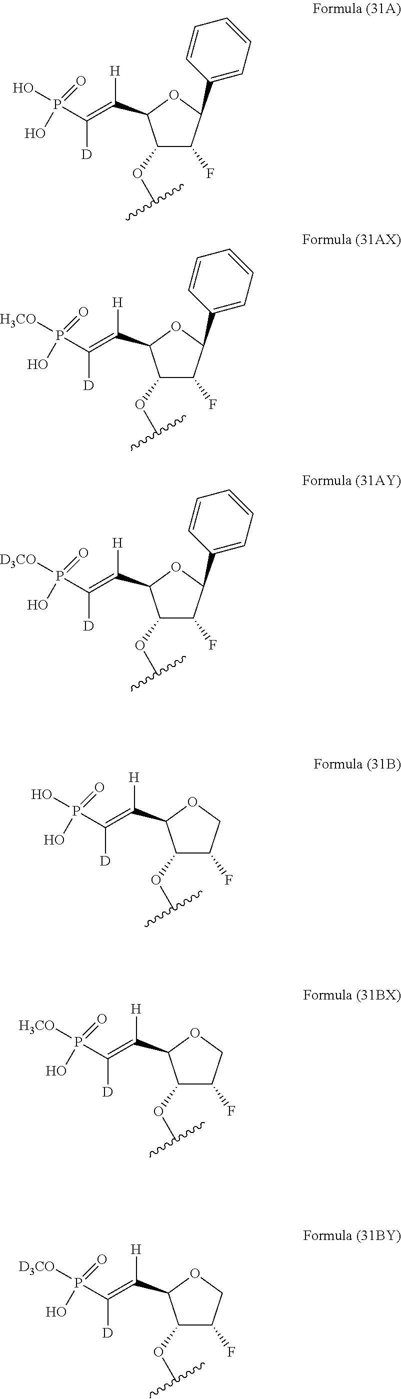

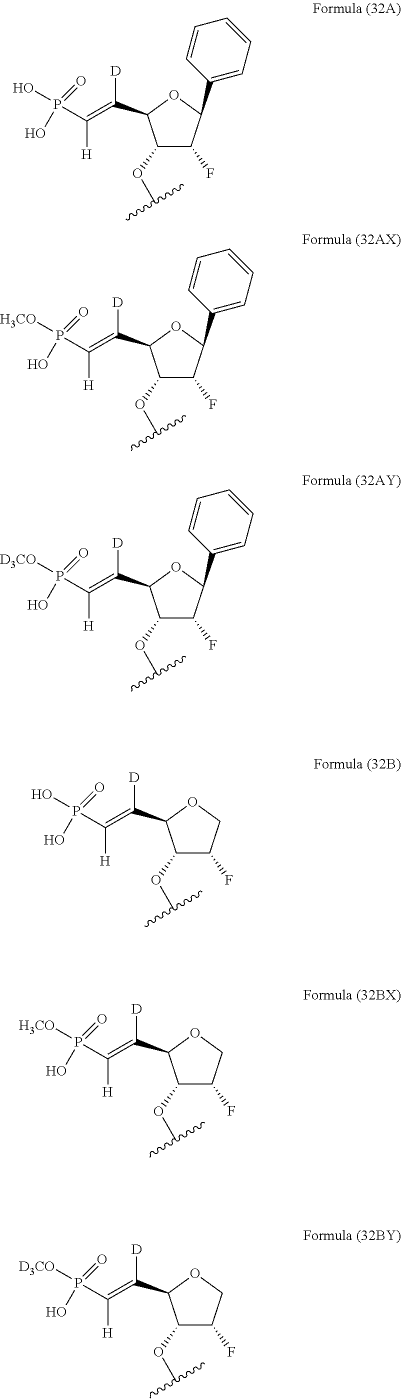

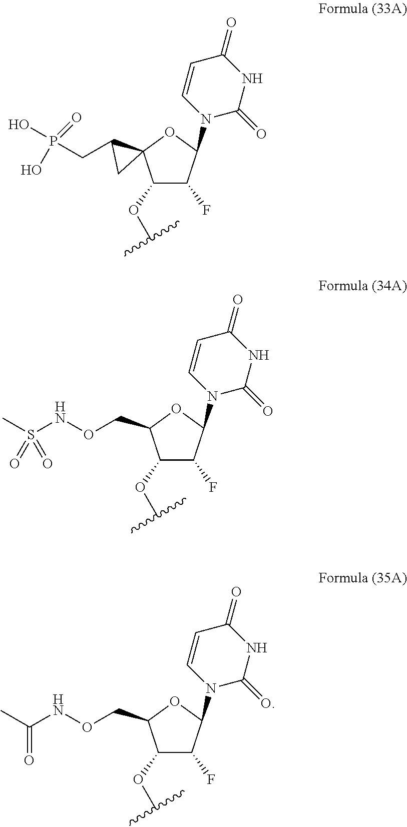

In some embodiments of the disclosed siNA molecules, the antisense strand comprises a 5′-stabilized end cap selected from the group consisting of Formulas (71A)-(86A), Formulas (79XA)-(82XA), Formulas (79YA)-(82YA); Formula (86XA), Formula (86X′A), Formula (86Y), and Formula (86Y′):

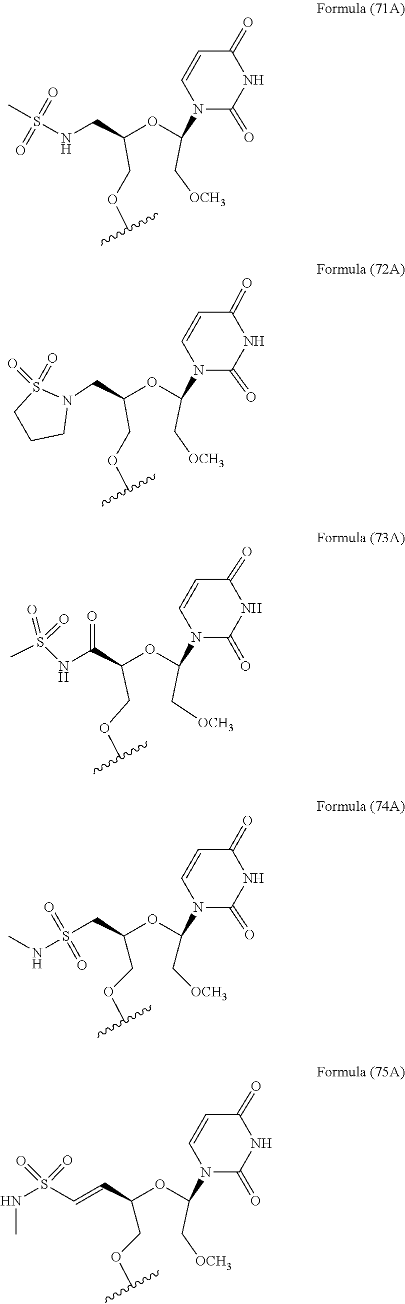 [SMILES: *OC[C@@H](CNS(C)(=O)=O)O[C@H](COC)n1ccc(=O)[nH]c1=O ; *OC[C@@H](CN1CCCS1(=O)=O)O[C@H](COC)n1ccc(=O)[nH]c1=O ; *OC[C@H](O[C@H](COC)n1ccc(=O)[nH]c1=O)C(=O)NS(C)(=O)=O ; *OC[C@@H](CS(=O)(=O)NC)O[C@H](COC)n1ccc(=O)[nH]c1=O ; *OC[C@@H](/C=C/S(=O)(=O)NC)O[C@H](COC)n1ccc(=O)[nH]c1=O]

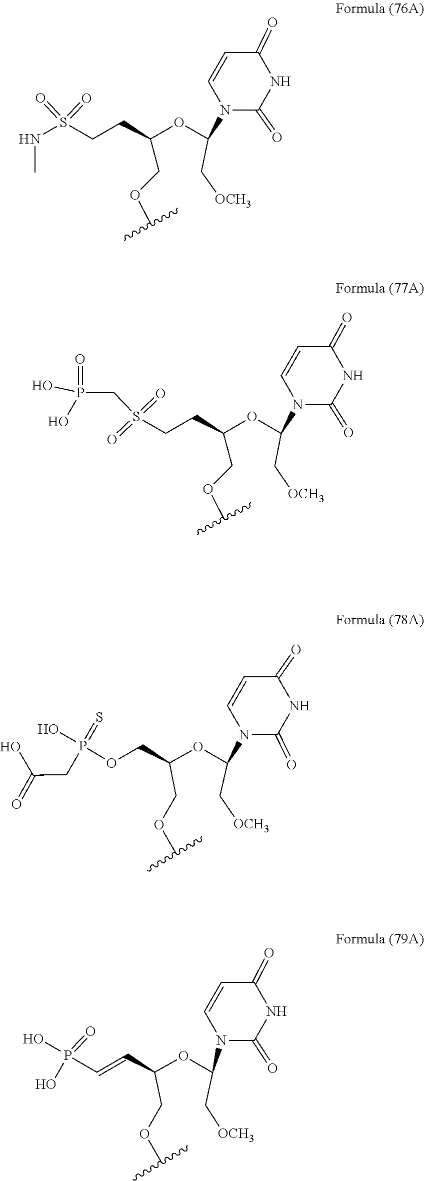 [SMILES: *OC[C@@H](CCS(=O)(=O)NC)O[C@H](COC)n1ccc(=O)[nH]c1=O ; *OC[C@@H](CCS(=O)(=O)CP(=O)(O)O)O[C@H](COC)n1ccc(=O)[nH]c1=O ; *OC[C@@H](COP(O)(=S)CC(=O)O)O[C@H](COC)n1ccc(=O)[nH]c1=O ; *OC[C@@H](/C=C/P(=O)(O)O)O[C@H](COC)n1ccc(=O)[nH]c1=O]

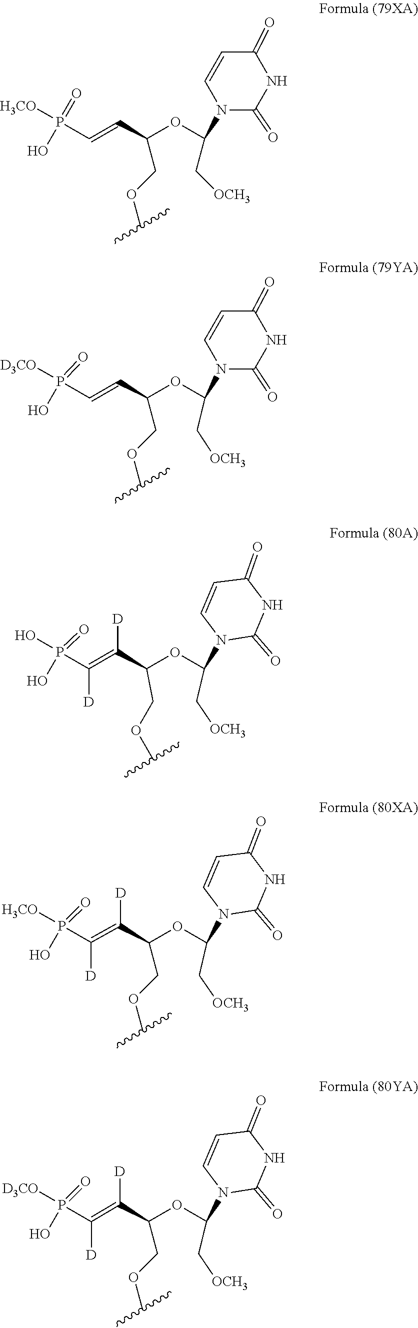

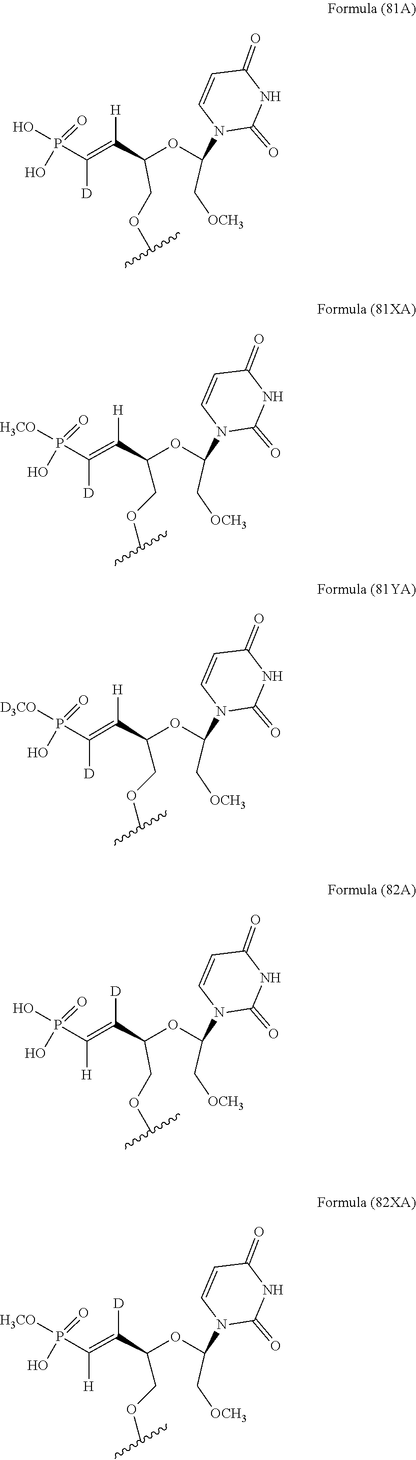

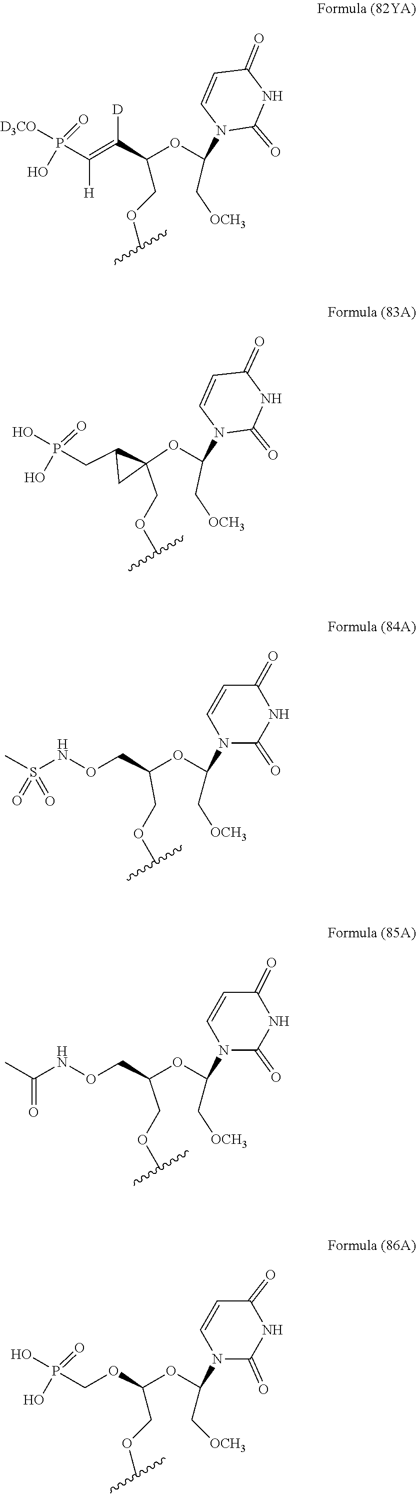

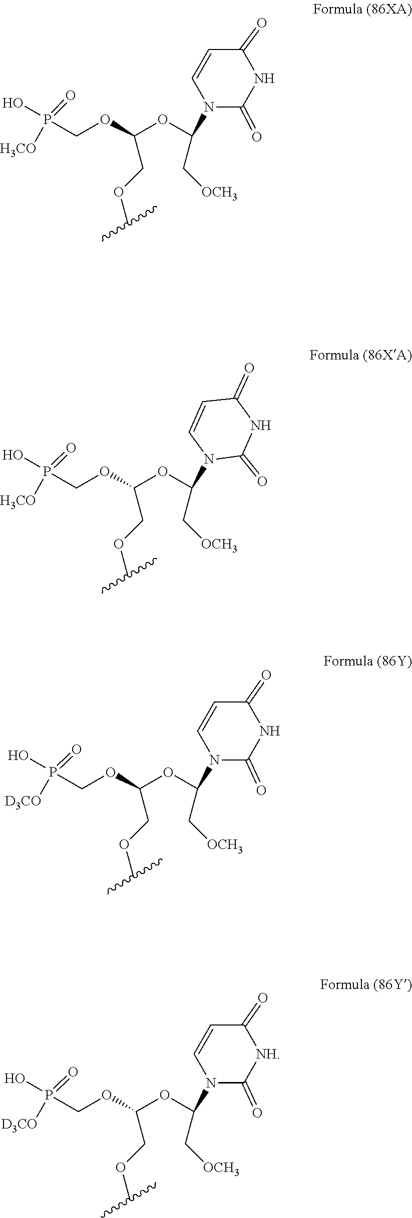

In some embodiments of the disclosed siNA molecules, the siNA further comprises a galactosamine. In some embodiments, the galactosamine is N-acetylgalactosamine (GalNAc) of Formula (VI):

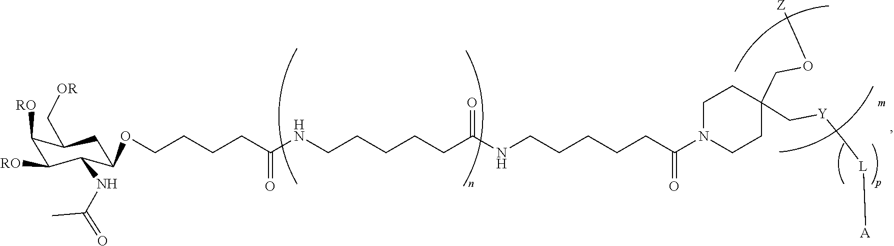

wherein
m is 1, 2, 3, 4, or 5;
each n is independently 1 or 2;
p is 0 or 1;
each R is independently H;
each Y is independently selected from —O—P(═O)(SH)—, —O—P(═O)(O)—, —O—P(═O)(OH)—, and —O—P(S)S—;
Z is H or a second protecting group;
either L is a linker or L and Y in combination are a linker; and
A is H, OH, a third protecting group, an activated group, or an oligonucleotide.

In some embodiments of the disclosed siNA molecules, the galactosamine is N-acetylgalactosamine (GalNAc) of Formula (VII):

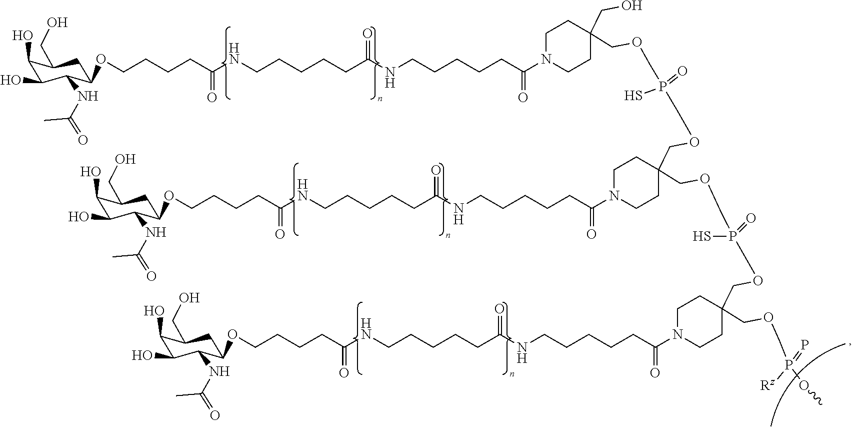

wherein R^{z} is OH or SH; and each n is independently 1 or 2.

In some embodiments of the disclosed siNA molecules, (i) at least one end of the siNA is a blunt end; (ii) at least one end of the siNA comprises an overhang, wherein the overhang comprises at least one nucleotide; or (iii) both ends of the siNA comprise an overhang, wherein the overhang comprises at least one nucleotide.

In some embodiments of the disclosed siNA molecules, (i) the target gene is a viral gene; (ii) the target gene is a gene is from a DNA virus; (iii) the target gene is a gene from a double-stranded DNA (dsDNA) virus; (iv) the target gene is a gene from a hepadnavirus; (v) the target gene is a gene from a a hepatitis B virus (HBV); (vi) the target gene is a gene from a HBV of any one of genotypes A-J; or (vii) the target gene is selected from the S gene or X gene of a HBV

The present disclosure provides siNA shown in Table 1, Table 2, Table 3, Table 4, and Table 5.

The present disclosure provides compositions comprising the siNA as disclosed herein; and a pharmaceutically acceptable excipient. In some embodiments, the compositions may further comprise 2, 3, 4, 5, 6, 7, 8, 9, 10 or more siNAs as disclosed herein. In some embodiments, the compositions may further comprise an additional treatment agent. For example, the additional treatment agent is selected from a nucleotide analog, nucleoside analog, a capsid assembly modulator (CAM), a recombinant interferon, an entry inhibitor, a small molecule immunomodulatory, and oligonucleotide therapy, such as an additional siNA, an antisense oligonucleotide (ASO), NAPs, or STOPS™

The present disclosure provides methods of treating a disease in a subject in need thereof, comprising administering to the subject the siNA disclosed herein or a composition comprising the siNA disclosed herein. The present disclosure further provides uses of the disclosed siNA and compositions for treating a disease in a subject. The present disclosure further provides siNA and compositions for use in treating a disease in a subject.

In some embodiments of the disclosed methods and uses, the disease is a viral disease, which is optionally caused by a DNA virus or a a double stranded DNA (dsDNA) virus. In some embodiments, the dsDNA virus is a hepadnavirus. In some embodiments, the hepadnavirus is a hepatitis B virus (HBV), and optionally wherein the HBV is selected from HBV genotypes A-J. In some embodiments, the methods and uses may further comprise administering an additional HBV treatment agent. In some embodiments, the siNA or the composition and the additional HBV treatment agent are administered concurrently or administered sequentially. In some embodiments, the additional HBV treatment agent is selected from a nucleotide analog, nucleoside analog, a capsid assembly modulator (CAM), a recombinant interferon, an entry inhibitor, a small molecule immunomodulator and oligonucleotide therapy. In some embodiments, the viral disease is a disease caused by a coronavirus, and optionally wherein the coronavirus is SARS-CoV-2.

In some embodiments of the disclosed methods and uses, the disease is a liver disease. In some embodiments, the liver disease is a nonalcoholic fatty liver disease (NAFLD) or hepatocellular carcinoma (HCC). In some embodiments, the NAFLD is nonalcoholic steatohepatitis (NASH). Some embodiments may further comprise administering to the subject a liver disease treatment agent. In some embodiments, the liver disease treatment agent is selected from a peroxisome proliferator-activator receptor (PPAR) agonist, farnesoid X receptor (FXR) agonist, lipid-altering agent, and incretin-based therapy. In some embodiments, (i) the PPAR agonist is selected from a PPARα agonist, dual PPARα/δ agonist, PPARγ agonist, and dual PPARα/γ agonist; (ii) the lipid-altering agent is aramchol; or (iii) the incretin-based therapy is a glucagon-like peptide 1 (GLP-1) receptor agonist or dipeptidyl peptidase 4 (DPP-4) inhibitor. In some embodiments, the siNA or composition and the liver disease treatment agent are administered concurrently or administered sequentially.

In some embodiments of the disclosed methods and uses, the siNA or the composition is administered at a dose of at least 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg 14 mg/kg, or 15 mg/kg.

In some embodiments of the disclosed methods and uses, the siNA or the composition is administered at a dose of between 0.5 mg/kg to 50 mg/kg, 0.5 mg/kg to 40 mg/kg 0.5 mg/kg to 30 mg/kg, 1 mg/kg to 50 mg/kg, 1 mg/kg to 40 mg/kg, 1 mg/kg to 30 mg/kg, 1 mg/kg to 20 mg/kg, 3 mg/kg to 50 mg/kg, 3 mg/kg to 40 mg/kg, 3 mg/kg to 30 mg/kg, 3 mg/kg to 20 mg/kg, 3 mg/kg to 15 mg/kg, 3 mg/kg to 10 mg/kg, 4 mg/kg to 50 mg/kg, 4 mg/kg to 40 mg/kg, 4 mg/kg to 30 mg/kg, 4 mg/kg to 20 mg/kg, 4 mg/kg to 15 mg/kg, 4 mg/kg to 10 mg/kg, 5 mg/kg to 50 mg/kg, 5 mg/kg to 40 mg/kg, 5 mg/kg to 30 mg/kg, 5 mg/kg to 20 mg/kg, 5 mg/kg to 15 mg/kg, or 5 mg/kg to 10 mg/kg.

In some embodiments of the disclosed methods and uses, the siNA or the composition is administered at least 1, 2, 3, 4, 5, 6, 7, 8, 9, or 10 times.

In some embodiments of the disclosed methods and uses, the siNA or the composition is administered at least 1, 2, 3, 4, 5, 6, 7, 8, 9, or 10 times a day, at least 1, 2, 3, 4, 5, 6, 7, 8, 9, or 10 times a week, or at least 1, 2, 3, 4, 5, 6, 7, 8, 9, or 10 times a month.

In some embodiments of the disclosed methods and uses, the siNA or the composition is administered at least once every 1, 2, 3, 4, 5, 6, 7, 8, 9, 10, 11, 12, 13, 14, 15, 16, 17, 18, 19, 20, or 21 days.

In some embodiments of the disclosed methods and uses, the siNA or the composition is administered for a period of at least 1, 2, 3, 4, 5, 6, 7, 8, 9, 10, 11, 12, 13, 14, 15, 16, 17, 18, 19, 20, or 21 days, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 51, 52, 53, 54, or 55 weeks.

In some embodiments of the disclosed methods and uses, the siNA or the composition is administered at a single dose of 5 mg/kg or 10 mg/kg, at three doses of 10 mg/kg once a week, at three doses of 10 mg/kg once every three days, or at five doses of 10 mg/kg once every three days.

In some embodiments of the disclosed methods and uses, the siNA or the composition is administered at six doses of ranging from 1 mg/kg to 15 mg/kg, 1 mg/kg to 10 mg/kg, 2 mg/kg to 15 mg/kg, 2 mg/kg to 10 mg/kg, 3 mg/kg to 15 mg/kg, or 3 mg/kg to 10 mg/kg; wherein the first dose and second dose are optionally administered at least 3 days apart; wherein the second dose and third dose are optionally administered at least 4 days apart; and wherein the third dose and fourth dose, fourth dose and fifth dose, and or fifth dose and sixth dose are optionally administered at least 7 days apart.

In some embodiments of the disclosed methods and uses, the siNA or the composition are administered in a particle or viral vector, wherein the viral vector is optionally selected from a vector of adenovirus, adeno-associated virus (AAV), alphavirus, flavivirus, herpes simplex virus, lentivirus, measles virus, picornavirus, poxvirus, retrovirus, and rhabdovirus. In some embodiments, the viral vector is a recombinant viral vector. In some embodiments, the viral vector is selected from AAVrh.74, AAVrh.10, AAVrh.20, AAV-1, AAV-2, AAV-3, AAV-4, AAV-5, AAV-6, AAV-7, AAV-8, AAV-9, AAV-10, AAV-11, AAV-12 and AAV-13.

In some embodiments of the disclosed methods and uses, the siNA or the composition is administered systemically or administered locally.

In some embodiments of the disclosed methods and uses, the siNA or the composition is administered intravenously, subcutaneously, or intramuscularly.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWINGS

FIG. 1 illustrates an exemplary siNA molecule.

FIG. 2 illustrates an exemplary siNA molecule.

FIGS. 3A-3H illustrate exemplary double-stranded siNA molecules.

FIG. 4 shows a graph of the change in serum HBsAg from AAV-HBV mice treated with vehicle (G01), CONTROL 2, ds-siNA-009, or ds-siNA-010.

FIG. 5A shows a graph of the change in serum HBsAg from AAV-HBV mice treated with vehicle (G01), CONTROL 2, ds-siNA-017 (with the addition of a GalNAc), or ds-siNA-018 (with the addition of a GalNAc).

FIG. 5B shows a graph of the change in serum HBsAg from AAV-HBV mice treated with vehicle (G01), CONTROL 2, CONTROL 7, or CONTROL 8.

FIG. 6 shows a graph of the change in serum HBsAg from AAV-HBV mice treated with vehicle (G01), CONTROL 2, ds-siNA-011, ds-siNA-012, or ds-siNA-013.

FIG. 7 shows shows a graph of the change in serum HBsAg from AAV-HBV mice treated with vehicle (G01), CONTROL 2, ds-siNA-026, ds-siNA-027, ds-siNA-028, ds-siNA-029, ds-siNA-030, ds-siNA-031, or ds-siNA-032.

FIG. 8 shows shows a graph of the change in serum HBsAg from AAV-HBV mice treated with vehicle (G01), CONTROL 2, ds-siNA-046, ds-siNA-047, ds-siNA-048, or ds-siNA-049.

### DETAILED DESCRIPTION

Disclosed herein are novel, modified nucelobase monomers that may comprise a unique chemical moiety in place of a base, lack a bond between the 3′ and 4′ carbons of the central furanose ring (i.e., an unlocked nucleotide), and/or possess a phosphate mimicking group (such nucleotides may henceforth be referred to as “nucleotide phosphate mimics”). Also disclosed herein are short interfering nucleic acid (siNA) molecules comprising modified nucleobases (i.e., nucleotides).

In general, the siNA molecules described herein may be double-stranded siNA (ds-siNA) molecules. The siNA molecules described herein may comprise modified nucleotides selected from 2′-O-methyl nucleotides and 2′-fluoro nucleotides. The siNA molecules described herein may comprise 1, 2, 3, 4, 5, 6, 7, 8, 9, or 10 or more phosphorothioate internucleoside linkages. The siNA molecules described herein may comprise 1, 2, 3, 4, 5, 6, 7, 8, 9, or 10 or more mesyl phosphoramidate internucleoside linkages. The siNA molecules described herein may comprise at least one phosphorylation blocker. The siNA molecules described herein may comprise a 5′-stabilized end cap (including but not limited to the disclosed nucleotide phosphate mimics). The siNA molecules described herein may comprise a galactosamine. The siNA molecules described herein may comprise one or more blunt ends. The siNA molecules described herein may comprise one or more overhangs.

For instance, the present disclosure provides modified nucleotides comprising a structure of:

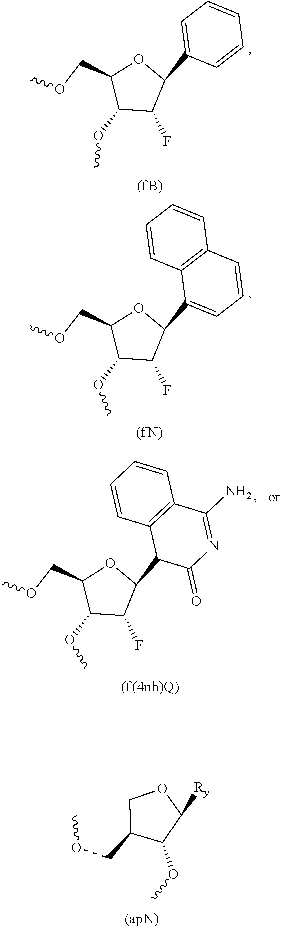

wherein R_{y} is a nucleobase, as well as modified nucleotides comprising a structure of:

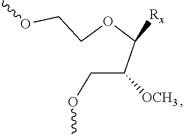

wherein Rₓ is a nucleobase, aryl, heteroaryl, or H. In some embodiments, the modified nucleotides may comprise a structure of:

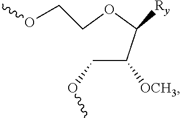

wherein R_{y} is a nucleobase. In some embodiments, the nucleobase is selected from thymine, cytosine, guanine, adenine, uracil, and an analogue or derivative thereof.

The present disclosure also provides nucleotide phosphate mimics that can serve as a stabilized end cap at the 5′ end of the antisense strand of any of the disclosed siNA. The disclosed nucleotide phosphate mimics include, but are not limited to, the structures:

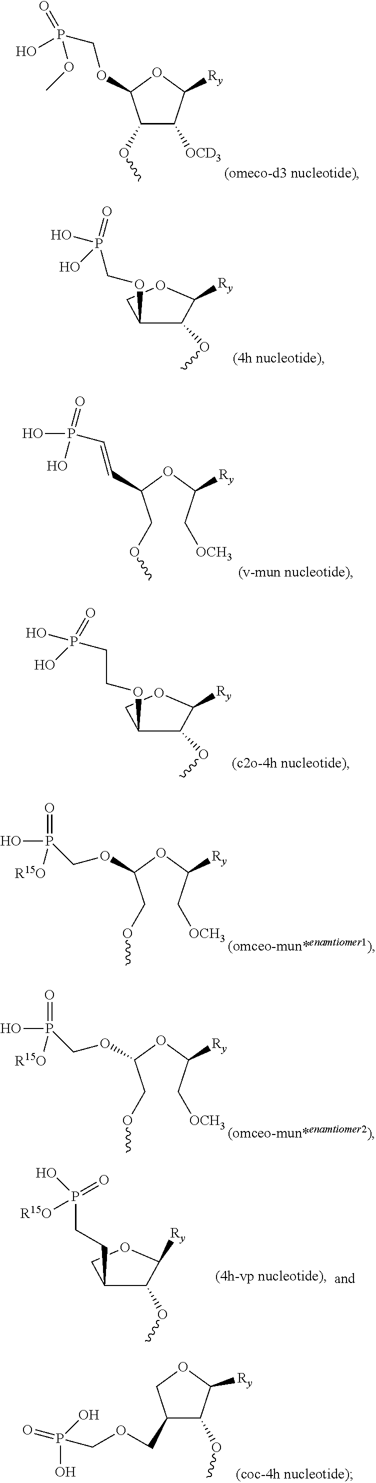

wherein R_{y} is a nucleobase and R¹⁵ is H or CH₃. In some embodiments, the nucleobase is selected from thymine, cytosine, guanine, adenine, uracil, and an analogue or derivative thereof. In some embodiments, the disclosed nucleotide phosphate mimics include, but are not limited to, the structures:

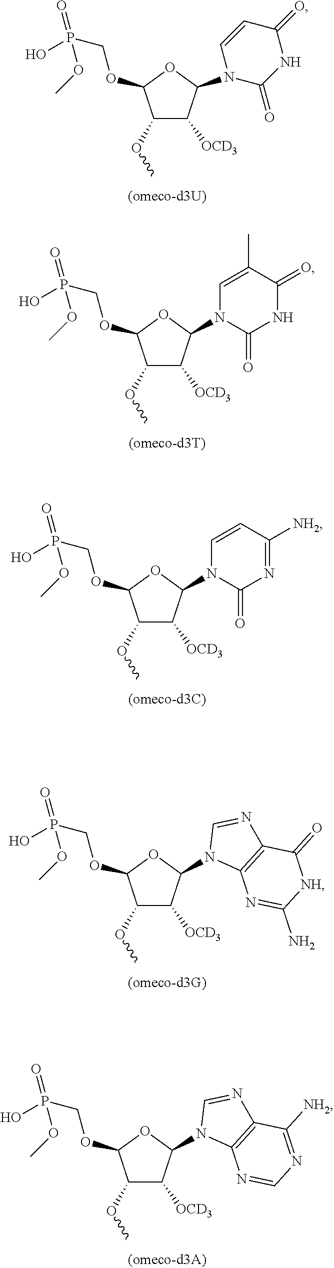

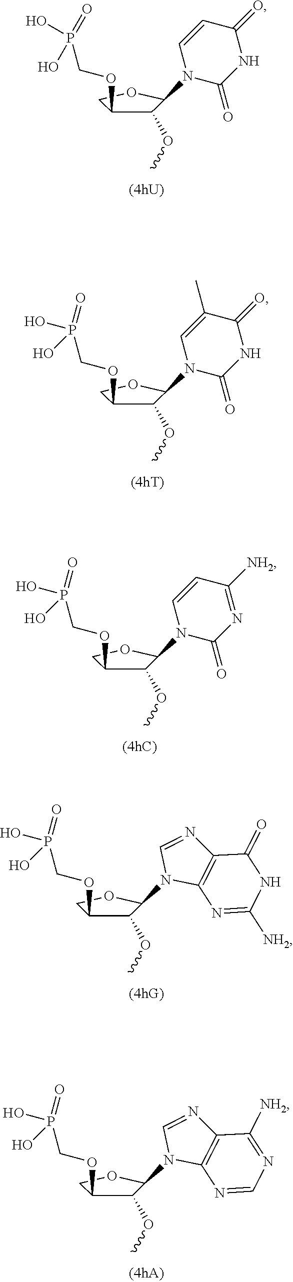

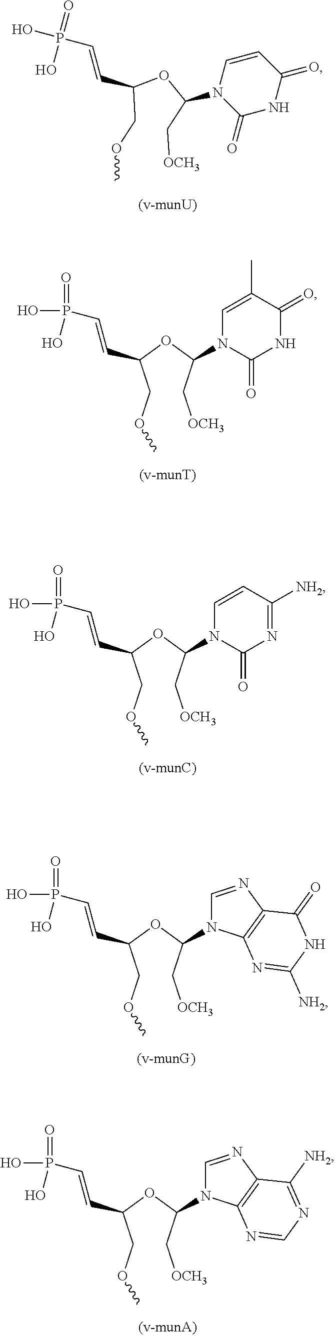

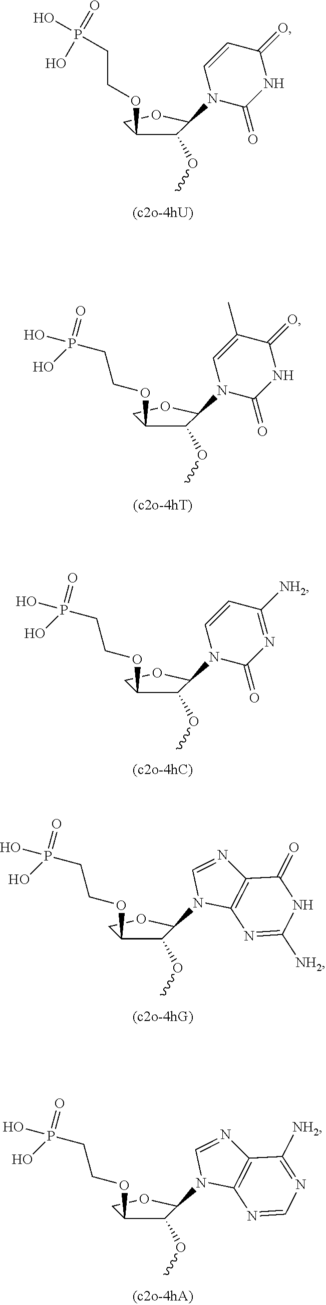

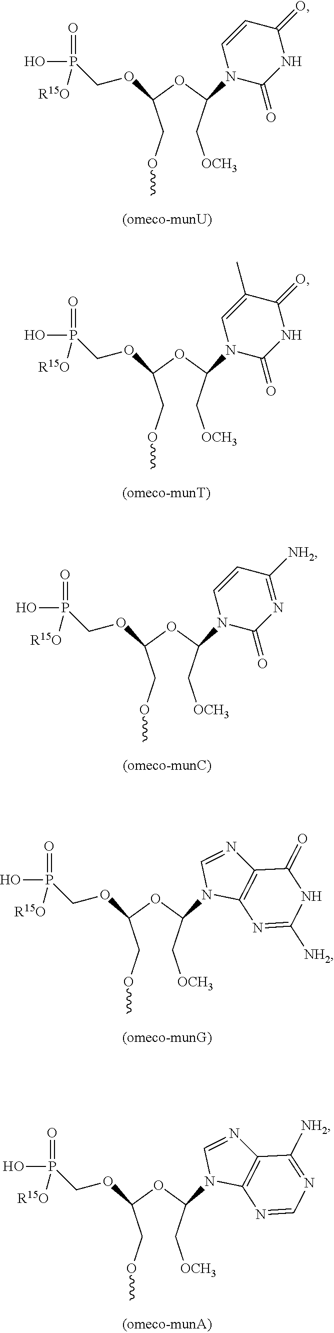

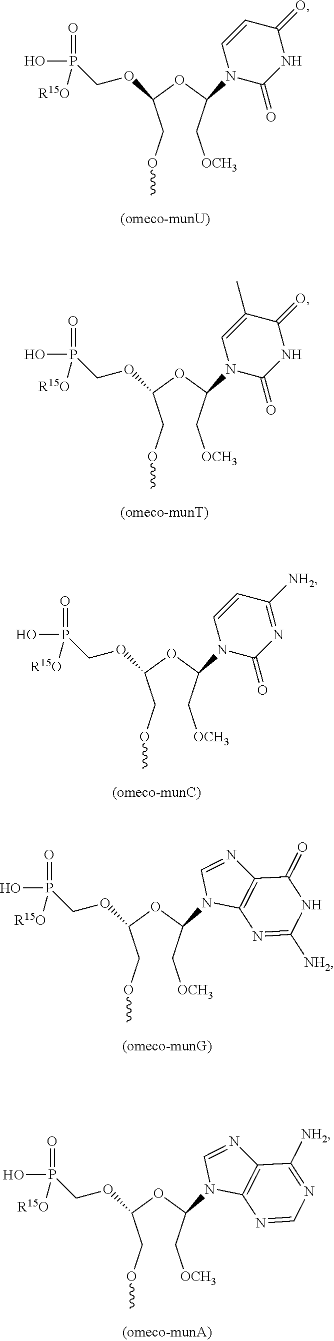

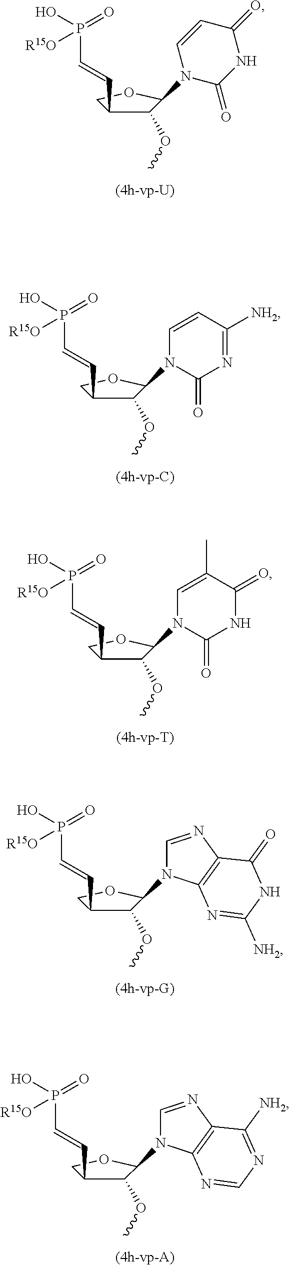

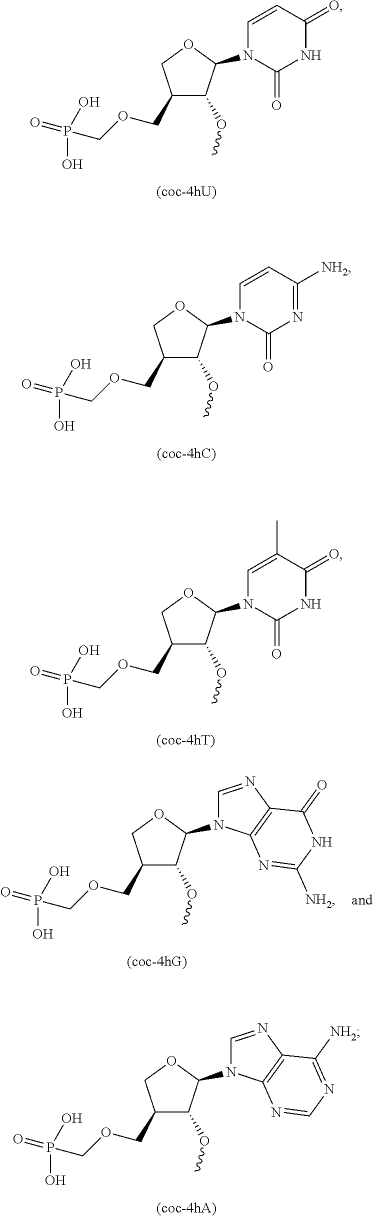

wherein R¹⁵ is H or CH₃.

The disclosed short interfering nucleic acid (siNA) molecules may comprise at least one, at least two, at least 3, at least 4, or at least 5 of the foregoing modified nucleotides and/or one of the foregoing nucleotide phosphate mimics at the 5′ end of the antisense strand. Indeed, a short interfering nucleic acid (siNA) molecule of the present disclosure may comprise:
(a) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence:
   (i) is 15 to 30 nucleotides in length; and
   (ii) comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 3, 5, 7, 8, 9, 10, 11, 12, 14, 17, and/or 19 from the 5′ end of the first nucleotide sequence is a 2′-fluoro nucleotide or wherein at least one modified nucleotide is a 2′-O-methyl nucleotide and at least one modified nucleotide is a 2′-fluoro nucleotide; and
   an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence:
   (iii) is 15 to 30 nucleotides in length; and
   (iv) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; or
(b) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence:
   (i) is 15 to 30 nucleotides in length; and
   (ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; and
an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence:
   (iii) is 15 to 30 nucleotides in length; and
   (iv)comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 2, 5, 6, 8, 10, 14, 16, 17, and/or 18 from the 5′ end of the second nucleotide sequence is a 2′-fluoro nucleotide; or
(c) a sense strand comprising a first nucleotide sequence that is at least about 60%, 65%, 70%, 75%, 80%, 85%, 90%, 95%, or 100% identical to an RNA corresponding to a target gene, wherein the first nucleotide sequence:
   (i) is 15 to 30 nucleotides in length; and
   (ii) comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 3, 5, 7, 8, 9, 10, 11, 12, 14, 17, and/or 19 from the 5′ end of the first nucleotide sequence is a 2′-fluoro nucleotide or wherein at least one modified nucleotide is a 2′-O-methyl nucleotide and at least one modified nucleotide is a 2′-fluoro nucleotide; and
an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence:
   (iii) is 15 to 30 nucleotides in length; and
   (iv) comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 2, 5, 6, 8, 10, 14, 16, 17, and/or 18 from the 5′ end of the second nucleotide sequence is a 2′-fluoro nucleotide;
      so long as the sense strand and/or the antisense strand comprise at least one, at least two, at least 3, at least 4, or at least 5 of the modified nucleotide(s) selected from

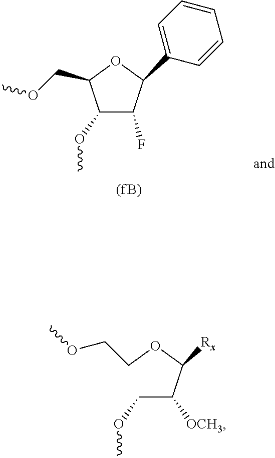

wherein Rx is a nucleobase, aryl, heteroaryl, or H; and/or so long as the antisense strand comprises a nucleotide phosphate mimic selected from:

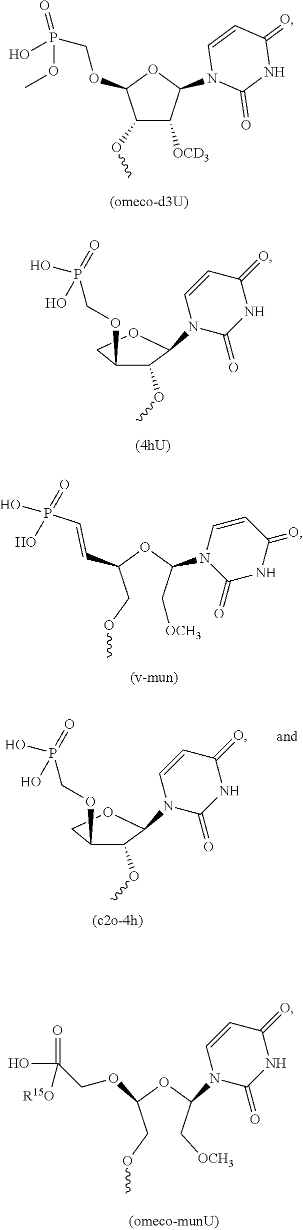

where R¹⁵ is H or CH₃.

Further, the siNA of the present disclosure may comprise a sense strand and/or an antisense strand that each independently comprise 1 or more phosphorothioate internucleoside linkages, 1 or more mesyl phosphoramidate internucleoside linkages, or a combination thereof. The siNA may comprise a phosphorylation blocker, a galactosamine, and/or a 5′-stabilized end cap (other than those noted above). The siNA may be conjugated to a targeting moiety, such as a galactosamine.

Further disclosed herein are compositions comprising two or more of the siNA molecules described herein.

Further disclosed herein are compositions comprising any of the siNA molecule described and a pharmaceutically acceptable carrier or diluent. Such compositions may also include an additional therapeutic agent, or may be administered in conjunction with an additional therapeutic agent (either concurrently or sequentially).

Further disclosed herein are compositions comprising two or more of the siNA molecules described herein for use as a medicament.

Further disclosed herein are compositions comprising any of the siNA molecule described and a pharmaceutically acceptable carrier or diluent for use as a medicament. Such medicaments may also include an additional therapeutic agent, or may be administered in conjunction with an additional therapeutic agent (either concurrently or sequentially).

Further disclosed herein are methods of treating a disease in a subject in need thereof, the methods comprising administering to the subject any of the siNA molecules (or a combination thereof) or compositions/medicaments described herein.

Further disclosed herein are uses of any of the siNA molecules described herein (or a combination thereof) in the manufacture of a medicament for treating a disease.

Short Interfering Nucleic Acid (siNA) Molecules

As indicated above, the present disclosure provides siNA molecules comprising modified nucleotides. Any of the siNA molecules described herein may be double-stranded siNA (ds-siNA) molecules. The terms “siNA molecules” and “ds-siNA molecules” may be used interchangeably. In some embodiments, the ds-siNA molecules comprise a sense strand and an antisense strand.

For the purposes of the present disclosure, the siNA molecules disclosed herein may generally comprise (a) at least one phosphorylation blocker, conjugated moiety, and/or 5′-stabilized end cap; and (b) a short interfering nucleic acid (siNA). In some embodiments, the phosphorylation blocker is a phosphorylation blocker disclosed herein. In some embodiments, the conjugated moiety is a galactosamine disclosed herein. In some embodiments, the 5′-stabilized end cap is a 5′-stabilized end cap disclosed herein.

The siNA may comprise any of the first nucleotide, second nucleotide, sense strand, or antisense strand sequences disclosed herein. The siNA may comprise 5 to 100, 5 to 90, 10 to 100, 10 to 90, 10 to 80, 10 to 70, 10 to 60, 10 to 50, 10 to 30, 10 to 25, 15 to 100, 15 to 90, 15 to 80, 15 to 70, 15 to 60, 15 to 50, 15 to 30, or 15 to 25 nucleotides. The siNA may comprise at least 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides. The siNA may comprise less than or equal to 50, 45, 40, 39, 38, 37, 36, 35, 34, 33, 32, 31, 30, 29, 28, 27, 26, 25, 24, 23, 22, 21, 20, or 19 nucleotides. The nucleotides may be modified nucleotides. The siNA may be single stranded (ss-siNA). The siNA may be double stranded (ds-siNA).

The ds-siNA may comprise (a) a sense strand comprising 15 to 30, 15 to 25, 15 to 24, 15 to 23, 15 to 22, 15 to 21, 17 to 30, 17 to 25, 17 to 24, 17 to 23, 17 to 22, 17 to 21, 18 to 30, 18 to 25, 18 to 24, 18 to 23, 18 to 22, 18 to 21, 19 to 30, 19 to 25, 19 to 24, 19 to 23, 19 to 22, 19 to 21, 20 to 25, 20 to 24, 20 to 23, 21 to 25, 21 to 24, or 21 to 23 nucleotides; and (b) an antisense strand comprising 15 to 30, 15 to 25, 15 to 24, 15 to 23, 15 to 22, 15 to 21, 17 to 30, 17 to 25, 17 to 24, 17 to 23, 17 to 22, 17 to 21, 18 to 30, 18 to 25, 18 to 24, 18 to 23, 18 to 22, 18 to 21, 19 to 30, 19 to 25, 19 to 24, 19 to 23, 19 to 22, 19 to 21, 20 to 25, 20 to 24, 20 to 23, 21 to 25, 21 to 24, or 21 to 23 nucleotides. The ds-siNA may comprise (a) a sense strand comprising about 15, 16, 17, 18, 19, 20, 21, 22, or 23 nucleotides; and (b) an antisense strand comprising about 15, 16, 17, 18, 19, 20, 21, 22, or 23 nucleotides. The ds-siNA may comprise (a) a sense strand comprising about 19 nucleotides; and (b) an antisense strand comprising about 21 nucleotides. The ds-siNA may comprise (a) a sense strand comprising about 21 nucleotides; and (b) an antisense strand comprising about 23 nucleotides.

Any of the siNA molecules disclosed herein may further comprise one or more linkers independently selected from a phosphodiester (PO) linker, phosphorothioate (PS) linker, phosphorodithioate linker, mesyl phosphoramidate (Ms), and PS-mimic linker. In some embodiments, the PS-mimic linker is a sulfur linker. In some embodiments, the linkers are internucleoside linkers. Alternatively or additionally, the linkers may connect a nucleotide of the siNA molecule to at least one phosphorylation blocker, conjugated moiety, or 5′-stabilized end cap. In some embodiments, the linkers connect a conjugated moiety to a phosphorylation blocker or 5′-stabilized end cap.

An exemplary siNA molecule of the present disclosure is shown in FIG. 1. As shown in FIG. 1, an exemplary siNA molecule comprises a sense strand (101) and an antisense strand (102). The sense strand (101) may comprise a first oligonucleotide sequence (103). The first oligonucleotide sequence (103) may comprise one or more phosphorothioate internucleoside linkages (109). The phosphorothioate internucleoside linkage (109) may be between the nucleotides at the 5′ or 3′ terminal end of the first oligonucleotide sequence (103). The phosphorothioate internucleoside linkage (109) may be between the first three nucleotides from the 5′ end of the first oligonucleotide sequence (103). The first oligonucleotide sequence (103) may comprise one or more 2′-fluoro nucleotides (110). The first oligonucleotide sequence (103) may comprise one or more 2′-O-methyl nucleotides (111). The first oligonucleotide sequence (103) may comprise 15 or more modified nucleotides independently selected from 2′-fluoro nucleotides (110) and 2′-O-methyl nucleotides (111). The sense strand (101) may further comprise a phosphorylation blocker (105). The sense strand (101) may further comprise a galactosamine (106). The antisense strand (102) may comprise a second oligonucleotide sequence (104). The second oligonucleotide sequence (104) may comprise one or more phophorothioate internucleoside linkages (109). The phosphorothioate internucleoside linkage (109) may be between the nucleotides at the 5′ or 3′ terminal end of the second oligonucleotide sequence (104). The phosphorothioate internucleoside linkage (109) may be between the first three nucleotides from the 5′ end of the second oligonucleotide sequence (104). The phosphorothioate internucleoside linkage (109) may be between the first three nucleotides from the 3′ end of the second oligonucleotide sequence (104). The second oligonucleotide sequence (104) may comprise one or more 2′-fluoro nucleotides (110). The second oligonucleotide sequence (104) may comprise one or more 2′-O-methyl nucleotides (111). The second oligonucleotide sequence (104) may comprise 15 or more modified nucleotides independently selected from 2′-fluoro nucleotides (110) and 2′-O-methyl nucleotides (111). The antisense strand (102) may further comprise a 5′-stabilized end cap (107). The siNA may further comprise one or more blunt ends. Alternatively, or additionally, one end of the siNA may comprise an overhang (108). The overhang (108) may be part of the sense strand (101). The overhang (108) may be part of the antisense strand (102). The overhang (108) may be distinct from the first nucleotide sequence (103). The overhang (108) may be distinct from the second nucleotide sequence (104). The overhang (108) may be part of the first nucleotide sequence (103). The overhang (108) may be part of the second nucleotide sequence (104). The overhang (108) may comprise 1 or more nucleotides. The overhang (108) may comprise 1 or more deoxyribonucleotides. The overhang (108) may comprise 1 or more modified nucleotides. The overhang (108) may comprise 1 or more modified ribonucleotides. The sense strand (101) may be shorter than the antisense strand (102). The sense strand (101) may be the same length as the antisense strand (102). The sense strand (101) may be longer than the antisense strand (102).

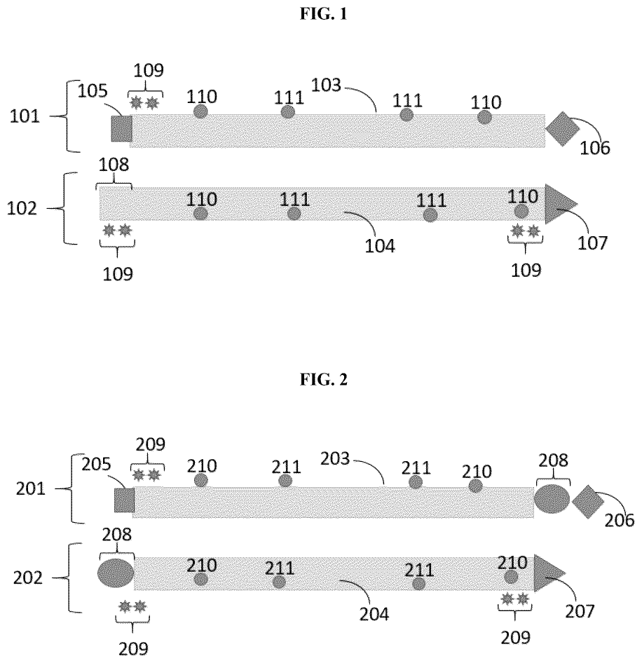

An exemplary siNA molecule of the present disclosure is shown in FIG. 2. As shown in FIG. 2, an exemplary siNA molecule comprises a sense strand (201) and an antisense strand (202). The sense strand (201) may comprise a first oligonucleotide sequence (203). The first oligonucleotide sequence (203) may comprise one or more phophorothioate internucleoside linkages (209). The phosphorothioate internucleoside linkage (209) may be between the nucleotides at the 5′ or 3′ terminal end of the first oligonucleotide sequence (203). The phosphorothioate internucleoside linkage (209) may be between the first three nucleotides from the 5′ end of the first oligonucleotide sequence (203). The first oligonucleotide sequence (203) may comprise one or more 2′-fluoro nucleotides (210). The first oligonucleotide sequence (203) may comprise one or more 2′-O-methyl nucleotides (211). The first oligonucleotide sequence (203) may comprise 15 or more modified nucleotides independently selected from 2′-fluoro nucleotides (210) and 2′-O-methyl nucleotides (211). The sense strand (201) may further comprise a phosphorylation blocker (205). The sense strand (201) may further comprise a galactosamine (206). The antisense strand (202) may comprise a second oligonucleotide sequence (204). The second oligonucleotide sequence (204) may comprise one or more phophorothioate internucleoside linkages (209). The phosphorothioate internucleoside linkage (209) may be between the nucleotides at the 5′ or 3′ terminal end of the second oligonucleotide sequence (204). The phosphorothioate internucleoside linkage (209) may be between the first three nucleotides from the 5′ end of the second oligonucleotide sequence (204). The phosphorothioate internucleoside linkage (209) may be between the first three nucleotides from the 3′ end of the second oligonucleotide sequence (204). The second oligonucleotide sequence (204) may comprise one or more 2′-fluoro nucleotides (210). The second oligonucleotide sequence (204) may comprise one or more 2′-O-methyl nucleotides (211). The second oligonucleotide sequence (204) may comprise 15 or more modified nucleotides independently selected from 2′-fluoro nucleotides (210) and 2′-O-methyl nucleotides (211). The antisense strand (202) may further comprise a 5′-stabilized end cap (207). The siNA may further comprise one or more overhangs (208). The overhang (208) may be part of the sense strand (201). The overhang (208) may be part of the antisense strand. (202). The overhang (208) may be distinct from the first nucleotide sequence (203). The overhang (208) may be distinct from the second nucleotide sequence (204). The overhang (208) may be part of the first nucleotide sequence (203). The overhang (208) may be part of the second nucleotide sequence (204). The overhang (208) may be adjacent to the 3′ end of the first nucleotide sequence (203). The overhang (208) may be adjacent to the 5′ end of the first nucleotide sequence (203). The overhang (208) may be adjacent to the 3′ end of the second nucleotide sequence (204). The overhang (208) may be adjacent to the 5′ end of the second nucleotide sequence (204). The overhang (208) may comprise 1 or more nucleotides. The overhang (208) may comprise 1 or more deoxyribonucleotides. The overhang (208) may comprise a TT sequence. The overhang (208) may comprise 1 or more modified nucleotides. The overhang (208) may comprise 1 or more modified nucleotides disclosed herein (e.g., 2-fluoro nucleotide, 2′-O-methyl nucleotide, 2′-fluoro nucleotide mimic, 2′-O-methyl nucleotide mimic, or a nucleotide comprising a modified nucleobase). The overhang (208) may comprise 1 or more modified ribonucleotides. The sense strand (201) may be shorter than the antisense strand (202). The sense strand (201) may be the same length as the antisense strand (202). The sense strand (201) may be longer than the antisense strand (202).

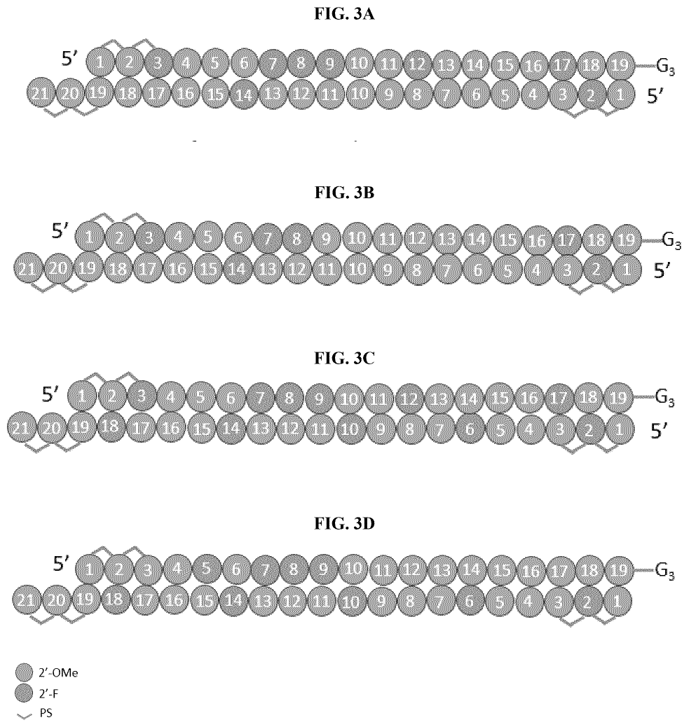

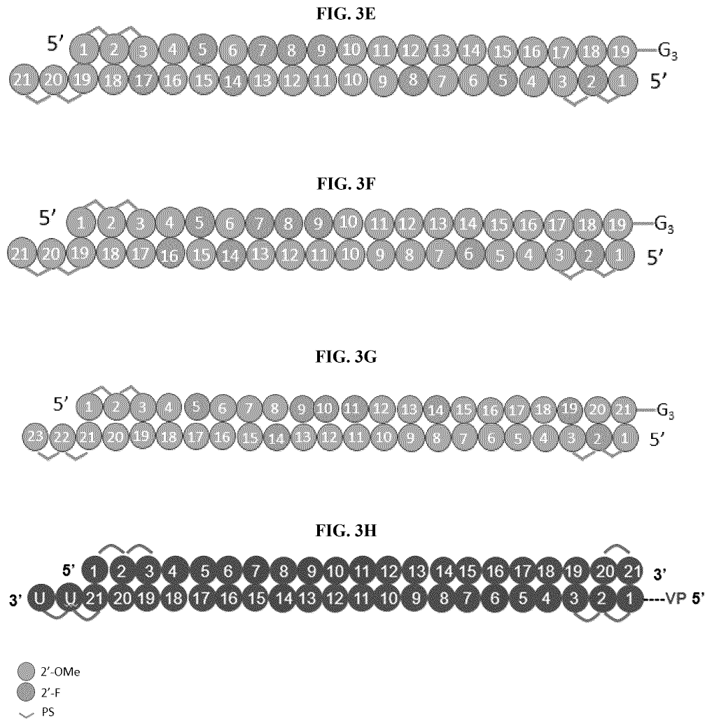

FIGS. 3A-3H depict exemplary ds-siNA modification patterns. As shown in FIGS. 3A-3G, an exemplary ds-siNA molecule may have the following formula:

5′-Aₙ¹Bₙ²Aₙ³Bₙ⁴Aₙ⁵Bₙ⁶Aₙ⁷Bₙ⁸Aₙ⁹-3′
3′-C_{q}¹A_{q}²B_{q}³A_{q}⁴B_{q}⁵A_{q}⁶B_{q}⁷A_{q}⁸B_{q}⁹A_{q}¹⁰B_{q}¹¹A_{q}¹²-5′

wherein:
the top strand is a sense strand comprising a first nucleotide sequence that is at least about 60%, 65%, 70%, 75%, 80%, 85%, 90%, 95%, or 100% identical to an RNA corresponding to a target gene, wherein the first nucleotide sequence comprises 15 to 30 nucleotides;
the bottom strand is an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence comprises 15 to 30 nucleotides;
each A is independently a 2′-O-methyl nucleotide or a nucleotide comprising a 5′
stabilized end cap or phosphorylation blocker;
B is a 2′-fluoro nucleotide;
C represents overhanging nucleotides and is a 2′-O-methyl nucleotide, a deoxy nucleotide, or uracil;
n¹=1-6 nucleotides in length;
each n², n⁶, n⁸, q³, q⁵, q⁷, q⁹, q¹¹, and q¹² is independently 0-1 nucleotides in length;
each n³ and n⁴ is independently 1-3 nucleotides in length;
n⁵ is 1-10 nucleotides in length;
n⁷ is 0-4 nucleotides in length;
each n⁹, q¹, and q² is independently 0-2 nucleotides in length;
q⁴ is 0-3 nucleotides in length;
q⁶ is 0-5 nucleotides in length;
q⁸ is 2-7 nucleotides in length; and
q¹⁰ is 2-11 nucleotides in length.
   The ds-siNA may further comprise a conjugated moiety. The conjugated moiety may comprise any of the galactosamines disclosed herein. The ds-siNA may further comprise (i) phosphorothioate internucleoside linkages between the nucleotides at positions 1 and 2 and positions 2 and 3 from the 5′ end of the sense strand; and (ii) phosphorothioate internucleoside linkages between the nucleotides at positions 1 and 2; positions 2 and 3; positions 19 and 20; and positions 20 and 21 from the 5′ end of the antisense strand. The ds-siNA may further comprise a 5′-stabilizing end cap. The 5′-stabilizing end cap may be a vinyl phosphonate. The 5′-stabilizing end cap may be attached to the 5′ end of the antisense strand. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker. An exemplary ds-siNA molecule may have the following formula:

5′-A₂₋₄B₁A₁₋₃B₂₋₃A₂₋₁₀B₀₋₁A₀₋₄B₀₋₁A₀₋₂-3′
3′-C₂A₀₋₂B₀₋₁A₀₋₃B₀₋₁A₀₋₅B₀₋₁A₂₋₇B₁A₂₋₁₁B₁A₁-5′

wherein:
the top strand is a sense strand comprising a first nucleotide sequence that is at least about 60%, 65%, 70%, 75%, 80%, 85%, 90%, 95%, or 100% identical to an RNA corresponding to a target gene, wherein the first nucleotide sequence comprises 15 to 30 nucleotides;
the bottom strand is an antisense strand comprising a second nucleotide sequence that is at least about 60%, 65%, 70%, 75%, 80%, 85%, 90%, 95%, or 100% complementary to the RNA corresponding to the target gene, wherein the second nucleotide sequence comprises 15 to 30 nucleotides;
each A is independently a 2′-O-methyl nucleotide or a nucleotide comprising a 5′
stabilized end cap or phosphorylation blocker;
B is a 2′-fluoro nucleotide;
C represents overhanging nucleotides and is a 2′-O-methyl nucleotide, a deoxy nucleotide, or uracil.
   The ds-siNA may further comprise a conjugated moiety. The conjugated moiety may comprise any of the galactosamines disclosed herein. The ds-siNA may further comprise (i) phosphorothioate internucleoside linkages between the nucleotides at positions 1 and 2 and positions 2 and 3 from the 5′ end of the sense strand; and (ii) phosphorothioate internucleoside linkages between the nucleotides at positions 1 and 2; positions 2 and 3; positions 19 and 20; and positions 20 and 21 from the 5′ end of the antisense strand. The ds-siNA may further comprise a 5′-stabilizing end cap. The 5′-stabilizing end cap may be a vinyl phosphonate. The vinyl phosphonate may be a deuterated vinyl phosphonate. The deuterated vinyl phosphonate may be a mono-deuterated vinyl phosphonate. The deuterated vinyl phosphonate may be a mono-di-deuterated vinyl phosphonate. The 5′-stabilizing end cap may be attached to the 5′ end of the antisense strand. The 5′-stabilizing end cap may be attached to the 3′ end of the antisense strand. The 5′-stabilizing end cap may be attached to the 5′ end of the sense strand. The 5′-stabilizing end cap may be attached to the 3′ end of the sense strand. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker.

The exemplary ds-siNA shown in FIGS. 3A-3H comprise (i) a sense strand comprising 19-21 nucleotides; and (ii) an antisense strand comprising 21-23 nucleotides. The ds-siNA may optionally further comprise (iii) a conjugated moiety, wherein the conjugated moiety (e.g., a GalNAc, noted as G3 in FIGS. 3A-3G) is attached to the 3′ end or the 5′ end of the sense strand or the antisense strand. The ds-siNA may comprise a 2 nucleotide overhang consisting of nucleotides at positions 20 and 21 from the 5′ end of the antisense strand. The ds-siNA may comprise a 2 nucleotide overhang consisting of nucleotides at positions 22 and 23 from the 5′ end of the antisense strand. The ds-siNA may further comprise 1, 2, 3, 4, 5, 6 or more phosphorothioate (ps) internucleoside linkages or mesyl phosphoramidate internucleoside linkage (Ms). At least one phosphorothioate internucleoside linkage or mesyl phosphoramidate internucleoside linkage (Ms) may be between the nucleotides at positions 1 and 2 or positions 2 and 3 from the 5′ end of the sense strand. At least one phosphorothioate internucleoside linkage or mesyl phosphoramidate internucleoside linkage (Ms) may be between the nucleotides at positions 1 and 2 or positions 2 and 3 from the 5′ end of the antisense strand. At least one phosphorothioate internucleoside linkage or mesyl phosphoramidate internucleoside linkage (Ms) may be between the nucleotides at positions 19 and 20, positions 20 and 21, positions 21 and 22, or positions 22 and 23 from the 5′ end of the antisense strand. As shown in FIGS. 3A-3H, 4-6 nucleotides in the sense strand may be 2′-fluoro nucleotides. As shown in FIGS. 3A-3H, 2-5 nucleotides in the antisense strand may be 2′-fluoro nucleotides. As shown in FIGS. 3A-3H, 13-15 nucleotides in the sense strand may be 2′-O-methyl nucleotides. As shown in FIGS. 3A-3H, 14-19 nucleotides in the antisense strand may be 2′-O-methyl nucleotides. As shown in FIGS. 3A-3H, the ds-siNA does not contain a base pair between 2′-fluoro nucleotides on the sense and antisense strands. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker.

As shown in FIG. 3A, a ds-siNA may comprise (a) a sense strand consisting of 19 nucleotides, wherein 2′-fluoro nucleotides are at positions 3, 7-9, 12, and 17 from the 5′ end of the sense strand, and wherein 2′-O-methyl nucleotides are at positions 1, 2, 4-6, 10, 11, 13-16, 18, and 19 from the 5′ end of the sense strand; (b) an antisense strand consisting of 21 nucleotides, wherein nucleotides at positions 2 and 14 from the 5′ end of the antisense strand are 2′-fluoro nucleotides; and wherein nucleotides at positions 1, 3-13, and 15-21 are 2′-O-methyl nucleotides. The ds-siNA may further comprise a conjugated moiety attached to the 3′ end of the sense strand. The ds-siNA may further comprise (i) phosphorothioate internucleoside linkages between the nucleotides at positions 1 and 2 and positions 2 and 3 from the 5′ end of the sense strand; and (ii) phosphorothioate internucleoside linkages between the nucleotides at positions 1 and 2; positions 2 and 3; positions 19 and 20; and positions 20 and 21 from the 5′ end of the antisense strand. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker. In some embodiments, the 2′-O-methyl nucleotide at position 1 from the 5′ end of the sense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, the 2′-O-methyl nucleotide at position 1 from the 5′ end of the antisense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, the 2′-O-methyl nucleotide at position 1 from the 3′ end of the sense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, the 2′-O-methyl nucleotide at position 1 from the 3′ end of the antisense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, at least 1, 2, 3, 4 or more 2′-fluoro nucleotides on the sense strand or antisense strand is a 2′-fluoro nucleotide mimic. In some embodiments, at least 1, 2, 3, 4 or more 2′-fluoro nucleotides on the sense strand or antisense strand is a fB, fN, f(4nh)Q, f4P, f2P, or fX nucleotide. In some embodiments, at least 1, 2, 3, 4 or more 2′-O-methyl nucleotide on the sense or antisense strand is a 2′-O-methyl nucleotide mimic. In some embodiments, one or more nucleotides in the sense strand and/or the antisense strand may be a 3′,4′ seco modified nucleotide in which the bond between the 3′ and 4′ positions of the furanose ring is broken (e.g., mun34).

As shown in FIG. 3B, a ds-siNA may comprise (a) a sense strand consisting of 19 nucleotides, wherein 2′-fluoro nucleotides are at positions 3, 7, 8, and 17 from the 5′ end of the sense strand, and wherein 2′-O-methyl nucleotides are at positions 1, 2, 4-6, 9-16, 18, and 19 from the 5′ end of the sense strand; (b) an antisense strand consisting of 21 nucleotides, wherein nucleotides at positions 2 and 14 from the 5′ end of the antisense strand are 2′-fluoro nucleotides; and wherein nucleotides at positions 1, 3-13, and 15-21 are 2′-O-methyl nucleotides. The ds-siNA may further comprise a conjugated moiety attached to the 3′ end of the sense strand. The ds-siNA may further comprise (i) phosphorothioate internucleoside linkages between the nucleotides at positions 1 and 2 and positions 2 and 3 from the 5′ end of the sense strand; and (ii) phosphorothioate internucleoside linkages between the nucleotides at positions 1 and 2; positions 2 and 3; positions 19 and 20; and positions 20 and 21 from the 5′ end of the antisense strand. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker. In some embodiments, the 2′-O-methyl nucleotide at position 1 from the 5′ end of the sense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, the 2′-O-methyl nucleotide at position 1 from the 5′ end of the antisense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, the 2′-O-methyl nucleotide at position 1 from the 3′ end of the sense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, the 2′-O-methyl nucleotide at position 1 from the 3′ end of the antisense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, at least 1, 2, 3, 4 or more 2′-fluoro nucleotides on the sense strand or antisense strand is a 2′-fluoro nucleotide mimic. In some embodiments, at least 1, 2, 3, 4 or more 2′-fluoro nucleotides on the sense strand or antisense strand is a fB, fN, f(4nh)Q, f4P, f2P, or fX nucleotide. In some embodiments, at least 1, 2, 3, 4 or more 2′-O-methyl nucleotide on the sense or antisense strand is a 2′-O-methyl nucleotide mimic. In some embodiments, one or more nucleotides in the sense strand and/or the antisense strand may be a 3′,4′ seco modified nucleotide in which the bond between the 3′ and 4′ positions of the furanose ring is broken (e.g., mun34).

As shown in FIG. 3C, a ds-siNA may comprise (a) a sense strand consisting of 19 nucleotides, wherein 2′-fluoro nucleotides are at positions 3, 7-9, 12 and 17 from the 5′ end of the sense strand, and wherein 2′-O-methyl nucleotides are at positions 1, 2, 4-6, 10, 11, 13-16, 18, and 19 from the 5′ end of the sense strand; (b) an antisense strand consisting of 21 nucleotides, wherein the nucleotides in the antisense strand comprise an alternating 1:3 modification pattern, and wherein 1 nucleotide is a 2′-fluoro nucleotide and 3 nucleotides are 2′-O-methyl nucleotides. The ds-siNA may further comprise a conjugated moiety attached to the 3′ end of the sense strand. The ds-siNA may further comprise (i) phosphorothioate internucleoside linkages between the nucleotides at positions 1 and 2 and positions 2 and 3 from the 5′ end of the sense strand; and (ii) phosphorothioate internucleoside linkages between the nucleotides at positions 1 and 2; positions 2 and 3; positions 19 and 20; and positions 20 and 21 from the 5′ end of the antisense strand. The ds-siNA may comprise 2-5 alternating 1:3 modification patterns on the antisense strand. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker. In some embodiments, the 2′-O-methyl nucleotide at position 1 from the 5′ end of the sense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, the 2′-O-methyl nucleotide at position 1 from the 5′ end of the antisense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, the 2′-O-methyl nucleotide at position 1 from the 3′ end of the sense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, the 2′-O-methyl nucleotide at position 1 from the 3′ end of the antisense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, at least 1, 2, 3, 4 or more 2′-fluoro nucleotides on the sense strand or antisense strand is a 2′-fluoro nucleotide mimic. In some embodiments, at least 1, 2, 3, 4 or more 2′-fluoro nucleotides on the sense strand is a fB, fN, f(4nh)Q, f4P, f2P, or fX nucleotide. In some embodiments, at least 1, 2, 3, 4 or more 2′-fluoro nucleotides on the antisense strand is a fB, fN, f(4nh)Q, f4P, f2P, or fX nucleotide. In some embodiments, at least 1, 2, 3, 4 or more 2′-O-methyl nucleotide on the sense or antisense strand is a 2′-O-methyl nucleotide mimic. In some embodiments, one or more nucleotides in the sense strand and/or the antisense strand may be a 3′,4′ seco modified nucleotide in which the bond between the 3′ and 4′ positions of the furanose ring is broken (e.g., mun34).

As shown in FIG. 3D, a ds-siNA may comprise (a) a sense strand consisting of 19 nucleotides, wherein 2′-fluoro nucleotides are at positions 5 and 7-9 from the 5′ end of the sense strand, and wherein 2′-O-methyl nucleotides are at positions 1-4, 6, and 10-19 from the 5′ end of the sense strand; (b) an antisense strand consisting of 21 nucleotides, wherein the nucleotides in the antisense strand comprise an alternating 1:3 modification pattern, and wherein 1 nucleotide is a 2′-fluoro nucleotide and 3 nucleotides are 2′-O-methyl nucleotides. The ds-siNA may further comprise a conjugated moiety attached to the 3′ end of the sense strand. The ds-siNA may further comprise (i) phosphorothioate internucleoside linkages between the nucleotides at positions 1 and 2 and positions 2 and 3 from the 5′ end of the sense strand; and (ii) phosphorothioate internucleoside linkages between the nucleotides at positions 1 and 2; positions 2 and 3; positions 19 and 20; and positions 20 and 21 from the 5′ end of the antisense strand. The ds-siNA may comprise 2-5 alternating 1:3 modification patterns on the antisense strand. The alternating 1:3 modification pattern may start at the nucleotide at any of positions 2, 6, 10, 14, and/or 18 from the 5′ end of the antisense strand. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker. In some embodiments, the 2′-O-methyl nucleotide at position 1 from the 5′ end of the sense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, the 2′-O-methyl nucleotide at position 1 from the 5′ end of the antisense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, the 2′-O-methyl nucleotide at position 1 from the 3′ end of the sense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, the 2′-O-methyl nucleotide at position 1 from the 3′ end of the antisense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, at least 1, 2, 3, 4 or more 2′-fluoro nucleotides on the sense strand or antisense strand is a 2′-fluoro nucleotide mimic. In some embodiments, at least 1, 2, 3, 4 or more 2′-fluoro nucleotides on the sense strand is a fB, fN, f(4nh)Q, f4P, f2P, or fX nucleotide. In some embodiments, at least 1, 2, 3, 4 or more 2′-fluoro nucleotides on the antisense strand is a fB, fN, f(4nh)Q, f4P, f2P, or fX nucleotide. In some embodiments, at least 1, 2, 3, 4 or more 2′-O-methyl nucleotide on the sense or antisense strand is a 2′-O-methyl nucleotide mimic. In some embodiments, one or more nucleotides in the sense strand and/or the antisense strand may be a 3′,4′ seco modified nucleotide in which the bond between the 3′ and 4′ positions of the furanose ring is broken (e.g., mun34).

As shown in FIG. 3E, a ds-siNA may comprise (a) a sense strand consisting of 19 nucleotides, wherein 2′-fluoro nucleotides are at positions 5 and 7-9 from the 5′ end of the sense strand, and wherein 2′-O-methyl nucleotides are at positions 1-4, 6, and 10-19 from the 5′ end of the sense strand; (b) an antisense strand consisting of 21 nucleotides, wherein the nucleotides in the antisense strand comprise an alternating 1:2 modification pattern, and wherein 1 nucleotide is a 2′-fluoro nucleotide and 2 nucleotides are 2′-O-methyl nucleotides. The ds-siNA may further comprise a conjugated moiety attached to the 3′ end of the sense strand. The ds-siNA may further comprise (i) phosphorothioate internucleoside linkages between the nucleotides at positions 1 and 2 and positions 2 and 3 from the 5′ end of the sense strand; and (ii) phosphorothioate internucleoside linkages between the nucleotides at positions 1 and 2; positions 2 and 3; positions 19 and 20; and positions 20 and 21 from the 5′ end of the antisense strand. The ds-siNA may comprise 2-5 alternating 1:2 modification patterns on the antisense strand. The alternating 1:2 modification pattern may start at the nucleotide at any of positions 2, 5, 8, 14, and/or 17 from the 5′ end of the antisense strand. In some embodiments, the ds-siNA comprises (a) a sense strand consisting of 19 nucleotides, wherein 2′-fluoro nucleotides are at positions 5 and 7-9 from the 5′ end of the sense strand, and wherein 2′-O-methyl nucleotides are at positions 1-4, 6, and 10-19 from the 5′ end of the sense strand; (b) an antisense strand consisting of 21 nucleotides, wherein 2′-fluoro nucleotides are at positions 2, 5, 8, 14, and 17 from the 5′ end of the antisense strand, and wherein 2′-O-methyl nucleotides are at positions 1, 3, 4, 6, 7, 9-13, 15, 16, and 18-21 from the 5′ end of the sense strand. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker. In some embodiments, the 2′-O-methyl nucleotide at position 1 from the 5′ end of the sense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, the 2′-O-methyl nucleotide at position 1 from the 5′ end of the antisense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, the 2′-O-methyl nucleotide at position 1 from the 3′ end of the sense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, the 2′-O-methyl nucleotide at position 1 from the 3′ end of the antisense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, at least 1, 2, 3, 4 or more 2′-fluoro nucleotides on the sense strand or antisense strand is a 2′-fluoro nucleotide mimic. In some embodiments, at least 1, 2, 3, 4 or more 2′-fluoro nucleotides on the sense strand is a fB, fN, f(4nh)Q, f4P, f2P, or fX nucleotide. In some embodiments, at least 1, 2, 3, 4 or more 2′-fluoro nucleotides on the antisense strand is a fB, fN, f(4nh)Q, f4P, f2P, or fX nucleotide. In some embodiments, at least 1, 2, 3, 4 or more 2′-O-methyl nucleotide on the sense or antisense strand is a 2′-O-methyl nucleotide mimic. In some embodiments, one or more nucleotides in the sense strand and/or the antisense strand may be a 3′,4′ seco modified nucleotide in which the bond between the 3′ and 4′ positions of the furanose ring is broken (e.g., mun34).

As shown in FIG. 3F, a ds-siNA may comprise (a) a sense strand consisting of 19 nucleotides, wherein 2′-fluoro nucleotides are at positions 5 and 7-9 from the 5′ end of the sense strand, and wherein 2′-O-methyl nucleotides are at positions 1-4, 6, and 10-19 from the 5′ end of the sense strand; (b) an antisense strand consisting of 21 nucleotides, wherein 2′-fluoro nucleotides are at positions 2, 6, 14, and 16 from the 5′ end of the antisense strand, and wherein 2′-O-methyl nucleotides are at positions 1, 3-5, 7-13, 15, and 17-21 from the 5′ end of the antisense strand. The ds-siNA may further comprise a conjugated moiety attached to the 3′ end of the sense strand. The ds-siNA may further comprise (i) phosphorothioate internucleoside linkages between the nucleotides at positions 1 and 2 and positions 2 and 3 from the 5′ end of the sense strand; and (ii) phosphorothioate internucleoside linkages between the nucleotides at positions 1 and 2; positions 2 and 3; positions 19 and 20; and positions 20 and 21 from the 5′ end of the antisense strand. In some embodiments, at least 1, 2, 3, 4 or more 2′-fluoro nucleotides on the sense strand or antisense strand is a fB, fN, f(4nh)Q, f4P, f2P, or fX nucleotide. In some embodiments, at least 1, 2, 3, 4 or more 2′-fluoro nucleotides on the sense strand or antisense strand is a f4P nucleotide. In some embodiments, at least 1, 2, 3, or 4 of the 2′-fluoro-nucleotides at positions 2, 6, 14, and 16 from the 5′ end of the antisense strand is a f4P nucleotide. In some embodiments, at least one of the 2′-fluoro-nucleotides at positions 2, 6, 14, and 16 from the 5′ end of the antisense strand is a f4P nucleotide. In some embodiments, at least two of the 2′-fluoro-nucleotides at positions 2, 6, 14, and 16 from the 5′ end of the antisense strand is a f4P nucleotide. In some embodiments, less than or equal to 3 of the 2′-fluoro-nucleotides at positions 2, 6, 14, and 16 from the 5′ end of the antisense strand is a f4P nucleotide. In some embodiments, less than or equal to 2 of the 2′-fluoro-nucleotides at positions 2, 6, 14, and 16 from the 5′ end of the antisense strand is a f4P nucleotide. In some embodiments, the 2′-fluoro-nucleotide at position 2 from the 5′ end of the antisense strand is a f4P nucleotide. In some embodiments, the 2′-fluoro-nucleotide at position 6 from the 5′ end of the antisense strand is a f4P nucleotide. In some embodiments, the 2′-fluoro-nucleotide at position 14 from the 5′ end of the antisense strand is a f4P nucleotide. In some embodiments, the 2′-fluoro-nucleotide at position 16 from the 5′ end of the antisense strand is a f4P nucleotide. In some embodiments, at least 1, 2, 3, 4 or more 2′-fluoro nucleotides on the sense strand or antisense strand is a f2P nucleotide. In some embodiments, at least 1, 2, 3, or 4 of the 2′-fluoro-nucleotides at positions 2, 6, 14, and 16 from the 5′ end of the antisense strand is a f2P nucleotide. In some embodiments, at least one of the 2′-fluoro-nucleotides at positions 2, 6, 14, and 16 from the 5′ end of the antisense strand is a f2P nucleotide. In some embodiments, at least two of the 2′-fluoro-nucleotides at positions 2, 6, 14, and 16 from the 5′ end of the antisense strand is a f2P nucleotide. In some embodiments, less than or equal to 3 of the 2′-fluoro-nucleotides at positions 2, 6, 14, and 16 from the 5′ end of the antisense strand is a f2P nucleotide. In some embodiments, less than or equal to 2 of the 2′-fluoro-nucleotides at positions 2, 6, 14, and 16 from the 5′ end of the antisense strand is a f2P nucleotide. In some embodiments, the 2′-fluoro-nucleotide at position 2 from the 5′ end of the antisense strand is a f2P nucleotide. In some embodiments, the 2′-fluoro-nucleotide at position 6 from the 5′ end of the antisense strand is a f2P nucleotide. In some embodiments, the 2′-fluoro-nucleotide at position 14 from the 5′ end of the antisense strand is a f2P nucleotide. In some embodiments, the 2′-fluoro-nucleotide at position 16 from the 5′ end of the antisense strand is a f2P nucleotide. In some embodiments, at least 1, 2, 3, 4 or more 2′-fluoro nucleotides on the sense strand or antisense strand is a fX nucleotide. In some embodiments, at least 1, 2, 3, or 4 of the 2′-fluoro-nucleotides at positions 2, 6, 14, and 16 from the 5′ end of the antisense strand is a fX nucleotide. In some embodiments, at least one of the 2′-fluoro-nucleotides at positions 2, 6, 14, and 16 from the 5′ end of the antisense strand is a fX nucleotide. In some embodiments, at least two of the 2′-fluoro-nucleotides at positions 2, 6, 14, and 16 from the 5′ end of the antisense strand is a fX nucleotide. In some embodiments, less than or equal to 3 of the 2′-fluoro-nucleotides at positions 2, 6, 14, and 16 from the 5′ end of the antisense strand is a fX nucleotide. In some embodiments, less than or equal to 2 of the 2′-fluoro-nucleotides at positions 2, 6, 14, and 16 from the 5′ end of the antisense strand is a fX nucleotide. In some embodiments, the 2′-fluoro-nucleotide at position 2 from the 5′ end of the antisense strand is a fX nucleotide. In some embodiments, the 2′-fluoro-nucleotide at position 6 from the 5′ end of the antisense strand is a fX nucleotide. In some embodiments, the 2′-fluoro-nucleotide at position 14 from the 5′ end of the antisense strand is a fX nucleotide. In some embodiments, the 2′-fluoro-nucleotide at position 16 from the 5′ end of the antisense strand is a fX nucleotide. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker. In some embodiments, the 2′-O-methyl nucleotide at position 1 from the 5′ end of the sense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, the 2′-O-methyl nucleotide at position 1 from the 5′ end of the antisense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, the 2′-O-methyl nucleotide at position 1 from the 3′ end of the sense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, the 2′-O-methyl nucleotide at position 1 from the 3′ end of the antisense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, at least 1, 2, 3, 4 or more 2′-fluoro nucleotides on the sense strand or antisense strand is a 2′-fluoro nucleotide mimic. In some embodiments, at least 1, 2, 3, 4 or more 2′-fluoro nucleotides on the sense strand is a fB, fN, f(4nh)Q, f4P, f2P, or fX nucleotide. In some embodiments, at least 1, 2, 3, 4 or more 2′-fluoro nucleotides on the antisense strand is a fB, fN, f(4nh)Q, f4P, f2P, or fX nucleotide. In some embodiments, at least 1, 2, 3, 4 or more 2′-O-methyl nucleotide on the sense or antisense strand is a 2′-O-methyl nucleotide mimic. In some embodiments, one or more nucleotides in the sense strand and/or the antisense strand may be a 3′,4′ seco modified nucleotide in which the bond between the 3′ and 4′ positions of the furanose ring is broken (e.g., mun34).

As shown in FIG. 3G, a ds-siNA may comprise (a) a sense strand consisting of 21 nucleotides, wherein 2′-fluoro nucleotides are at positions 5, 9-11, 14, and 19 from the 5′ end of the sense strand, and wherein 2′-O-methyl nucleotides are at positions 1-4, 6-8, 12, 13, 15-18, 20, and 21 from the 5′ end of the sense strand; and (b) an antisense strand consisting of 23 nucleotides, wherein 2′-flouro nucleodies are at positions 2 and 14 from the 5′ end of the antisense strand, and wherein 2′-O-methyl nucleotides are at positions 1, 3-13, and 15-23 from the 5′ end of the antisense strand. The ds-siNA may further comprise a conjugated moiety attached to the 3′ end of the sense strand. The ds-siNA may further comprise (i) phosphorothioate internucleoside linkages between the nucleotides at positions 1 and 2 and positions 2 and 3 from the 5′ end of the sense strand; and (ii) phosphorothioate internucleoside linkages between the nucleotides at positions 1 and 2; positions 2 and 3; positions 19 and 20; and positions 20 and 21 from the 5′ end of the antisense strand. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker. In some embodiments, the 2′-O-methyl nucleotide at position 1 from the 5′ end of the sense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, the 2′-O-methyl nucleotide at position 1 from the 5′ end of the antisense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, the 2′-O-methyl nucleotide at position 1 from the 3′ end of the sense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, the 2′-O-methyl nucleotide at position 1 from the 3′ end of the antisense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, at least 1, 2, 3, 4 or more 2′-fluoro nucleotides on the sense strand or antisense strand is a 2′-fluoro nucleotide mimic. In some embodiments, at least 1, 2, 3, 4 or more 2′-fluoro nucleotides on the sense strand is a fB, fN, f(4nh)Q, f4P, f2P, or fX nucleotide. In some embodiments, at least 1, 2, 3, 4 or more 2′-fluoro nucleotides on the antisense strand is a fB, fN, f(4nh)Q, f4P, f2P, or fX nucleotide. In some embodiments, at least 1, 2, 3, 4 or more 2′-O-methyl nucleotide on the sense or antisense strand is a 2′-O-methyl nucleotide mimic. In some embodiments, one or more nucleotides in the sense strand and/or the antisense strand may be a 3′,4′ seco modified nucleotide in which the bond between the 3′ and 4′ positions of the furanose ring is broken (e.g., mun34).

As shown in FIG. 3H, a ds-siNA may comprise (a) a sense strand consisting of 21 nucleotides, wherein 2′-fluoro nucleotides are at positions 7 and 9-11 from the 5′ end of the sense strand, and wherein 2′-O-methyl nucleotides are at positions 1-6, 8, and 12-21 from the 5′ end of the sense strand; and (b) an antisense strand consisting of 23 nucleotides, wherein 2′-flouro nucleodies are at positions 2, 6, 14, and 16 from the 5′ end of the antisense strand, and wherein 2′-O-methyl nucleotides are at positions 1, 3-5, 7-13, 15, and 17-23 from the 5′ end of the antisense strand. Optionally, the nucleotides at positions 22 and 23 of from the 5′ end of the antisense strand may be unlocked nucleotides. Optionally, the ds-siNA may further comprise a conjugated moiety attached to the 3′ end of the sense strand (not pictured). The ds-siNA may optionally comprise a vinyl phosphonate attached to the 5′ end of the antisense strand (pictured), but in some embodiments, a 5′ end cap disclosed herein may be suitable as well. The ds-siNA may further comprise (i) phosphorothioate internucleoside linkages between the nucleotides at positions 1 and 2, positions 2 and 3, and positions 20 and 21 from the 5′ end of the sense strand; and (ii) phosphorothioate internucleoside linkages between the nucleotides at positions 1 and 2; positions 2 and 3; positions 21 and 22, and positions 22 and 23 from the 5′ end of the antisense strand. In some embodiments, the 2′-O-methyl nucleotide at position 1 from the 5′ end of the sense strand is further modified to contain a 5′ stabilizing end cap. In some embodiments, the 2′-O-methyl nucleotide at position 1 from the 5′ end of the antisense strand is further modified to contain a 5′ stabilizing end cap. In some embodiments, the 2′-O-methyl nucleotide at position 1 from the 5′ end of the sense strand is further modified to contain a phosphorylation blocker. In some embodiments, the 2′-O-methyl nucleotide at position 1 from the 3′ end of the sense strand is further modified to contain a phosphorylation blocker. In some embodiments, the 2′-O-methyl nucleotide at position 1 from the 5′ end of the antisense strand is further modified to contain a phosphorylation blocker. In some embodiments, the 2′-O-methyl nucleotide at position 1 from the 3′ end of the antisense strand is further modified to contain a phosphorylation blocker. In some embodiments, the 2′-O-methyl nucleotide at position 1 from the 5′ end of the sense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide, a d2vd3U nucleotide, an omeco-d3U nucleotide, a 4hU nucleotide, a v-mun nucleotide, a c2o-4h nucleotide, an omeco-mun nucleotide, or a d2vmA nucleotide. In some embodiments, the 2′-O-methyl nucleotide at position 1 from the 5′ end of the antisense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, the 2′-O-methyl nucleotide at position 1 from the 3′ end of the sense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, the 2′-O-methyl nucleotide at position 1 from the 3′ end of the antisense strand is a d2vd3 nucleotide, a d2vd3U nucleotide, an omeco-d3 nucleotide, an omeco-d3U nucleotide, a 4h nucleotide, a 4hU nucleotide, a v-mun nucleotide, a c2o-4h nucleotide, an omeco-mun nucleotide, a d2vm nucleotide, or a d2vmA nucleotide. In some embodiments, at least 1, 2, 3, 4 or more 2′-fluoro nucleotides on the sense strand or antisense strand is a 2′-fluoro nucleotide mimic. In some embodiments, at least 1, 2, 3, 4 or more 2′-fluoro nucleotides on the sense strand is a fB, fN, f(4nh)Q, f4P, f2P, or fX nucleotide. In some embodiments, at least 1, 2, 3, 4 or more 2′-fluoro nucleotides on the antisense strand is a fB, fN, f(4nh)Q, f4P, f2P, or fX nucleotide. In some embodiments, at least 1, 2, 3, 4 or more 2′-O-methyl nucleotide on the sense or antisense strand is a 2′-O-methyl nucleotide mimic. In some embodiments, one or more nucleotides in the sense strand and/or the antisense strand may be a 3′,4′ seco modified nucleotide in which the bond between the 3′ and 4′ positions of the furanose ring is broken (e.g., mun34). siNA Sense Strand

Any of the siNA molecules described herein may comprise a sense strand. The sense strand may comprise a first nucleotide sequence. The first nucleotide sequence may be 15 to 30, 15 to 25, 15 to 23, 17 to 23, 19 to 23, or 19 to 21 nucleotides in length. In some embodiments, the first nucleotide sequence is 15, 16, 17, 18, 19, 20, 21, 22, 23, 24, 25, 26, 27, 28, 29, or 30 nucleotides in length. In some embodiments, the first nucleotide sequence is at least 19 nucleotides in length. In some embodiments, the first nucleotide sequence is at least 21 nucleotides in length.

In some embodiments, the sense strand is the same length as the first nucleotide sequence. In some embodiments, the sense strand is longer than the first nucleotide sequence. In some embodiments, the sense strand may further comprise 1, 2, 3, 4, or 5 or more nucleotides than the first nucleotide sequence. In some embodiments, the sense strand may further comprise a deoxyribonucleic acid (DNA). In some embodiments, the DNA is thymine (T). In some embodiments, the sense strand may further comprise a TT sequence. In some embodiments, the sense strand may further comprise one or more modified nucleotides that are adjacent to the first nucleotide sequence. In some embodiments, the one or more modified nucleotides are independently selected from any of the modified nucleotides disclosed herein (e.g., 2′-fluoro nucleotide, 2′-O-methyl nucleotide, 2′-fluoro nucleotide mimic, 2′-O-methyl nucleotide mimic, or a nucleotide comprising a modified nucleobase).

In some embodiments, the first nucleotide sequence comprises 15, 16, 17, 18, 19, 20, 21, 22, 23, or more modified nucleotides independently selected from a 2′-O-methyl nucleotide and a 2′-fluoro nucleotide. In some embodiments, 70%, 75%, 80%, 85%, 90%, 95% or 100% of the nucleotides in the first nucleotide sequence are modified nucleotides independently selected from a 2′-O-methyl nucleotide and a 2′-fluoro nucleotide. In some embodiments, 100% of the nucleotides in the first nucleotide sequence are modified nucleotides independently selected from a 2′-O-methyl nucleotide and a 2′-fluoro nucleotide. In some embodiments, the 2′-O-methyl nucleotide is a 2′-O-methyl nucleotide mimic. In some embodiments, the 2′-fluoro nucleotide is a 2′-fluoro nucleotide mimic.

In some embodiments, between about 15 to 30, 15 to 25, 15 to 24, 15 to 23, 15 to 22, 15 to 21, 17 to 30, 17 to 25, 17 to 24, 17 to 23, 17 to 22, 17 to 21, 18 to 30, 18 to 25, 18 to 24, 18 to 23, 18 to 22, 18 to 21, 19 to 30, 19 to 25, 19 to 24, 19 to 23, 19 to 22, 19 to 21, 20 to 25, 20 to 24, 20 to 23, 21 to 25, 21 to 24, or 21 to 23 modified nucleotides of the first nucleotide sequence are 2′-O-methyl nucleotides. In some embodiments, between about 2 to 20 modified nucleotides of the first nucleotide sequence are 2′-O-methyl nucleotides. In some embodiments, between about 5 to 25 modified nucleotides of the first nucleotide sequence are 2′-O-methyl nucleotides. In some embodiments, between about 10 to 25 modified nucleotides of the first nucleotide sequence are 2′-O-methyl nucleotides. In some embodiments, between about 12 to 25 modified nucleotides of the first nucleotide sequence are 2′-O-methyl nucleotides. In some embodiments, at least about 10, 11, 12, 13, 14, 15, 16, 17, 18, 19, 20, 21, or 22 modified nucleotides of the first nucleotide sequence are 2′-O-methyl nucleotides. In some embodiments, at least about 12 modified nucleotides of the first nucleotide sequence are 2′-O-methyl nucleotides. In some embodiments, at least about 13 modified nucleotides of the first nucleotide sequence are 2′-O-methyl nucleotides. In some embodiments, at least about 14 modified nucleotides of the first nucleotide sequence are 2′-O-methyl nucleotides. In some embodiments, at least about 15 modified nucleotides of the first nucleotide sequence are 2′-O-methyl nucleotides. In some embodiments, at least about 16 modified nucleotides of the first nucleotide sequence are 2′-O-methyl nucleotides. In some embodiments, at least about 17 modified nucleotides of the first nucleotide sequence are 2′-O-methyl nucleotides. In some embodiments, at least about 18 modified nucleotides of the first nucleotide sequence are 2′-O-methyl nucleotides. In some embodiments, at least about 19 modified nucleotides of the first nucleotide sequence are 2′-O-methyl nucleotides. In some embodiments, less than or equal to 25, 24, 23, 22, 21, 20, 19, 18, 17, 16, 15, 14, 13, 12, 11, 10, 9, 8, 7, 6, 5, 4, 3, or 2 modified nucleotides of the first nucleotide sequence are 2′-O-methyl nucleotides. In some embodiments, less than or equal to 21 modified nucleotides of the first nucleotide sequence are 2′-O-methyl nucleotides. In some embodiments, less than or equal to 20 modified nucleotides of the first nucleotide sequence are 2′-O-methyl nucleotides. In some embodiments, less than or equal to 19 modified nucleotides of the first nucleotide sequence are 2′-O-methyl nucleotides. In some embodiments, less than or equal to 18 modified nucleotides of the first nucleotide sequence are 2′-O-methyl nucleotides. In some embodiments, less than or equal to 17 modified nucleotides of the first nucleotide sequence are 2′-O-methyl nucleotides. In some embodiments, less than or equal to 16 modified nucleotides of the first nucleotide sequence are 2′-O-methyl nucleotides. In some embodiments, less than or equal to 15 modified nucleotides of the first nucleotide sequence are 2′-O-methyl nucleotides. In some embodiments, less than or equal to 14 modified nucleotides of the first nucleotide sequence are 2′-O-methyl nucleotides. In some embodiments, less than or equal to 13 modified nucleotides of the first nucleotide sequence are 2′-O-methyl nucleotides. In some embodiments, at least one modified nucleotide of the first nucleotide sequence is a 2′-O-methyl pyrimidine. In some embodiments, at least 5, 6, 7, 8, 9, or 10 modified nucleotides of the first nucleotide sequence are 2′-O-methyl pyrimidines. In some embodiments, at least one modified nucleotide of the first nucleotide sequence is a 2′-O-methyl purine. In some embodiments, at least 5, 6, 7, 8, 9, or 10 modified nucleotides of the first nucleotide sequence are 2′-O-methyl purines. In some embodiments, the 2′-O-methyl nucleotide is a 2′-O-methyl nucleotide mimic.

In some embodiments, between 2 to 15 modified nucleotides of the first nucleotide sequence are 2′-fluoro nucleotides. In some embodiments, between 2 to 10 modified nucleotides of the first nucleotide sequence are 2′-fluoro nucleotides. In some embodiments, between 2 to 6 modified nucleotides of the first nucleotide sequence are 2′-fluoro nucleotides. In some embodiments, 1 to 6, 1 to 5, 1 to 4, or 1 to 3 modified nucleotides of the first nucleotide sequence are 2′-fluoro nucleotides. In some embodiments, at least 1, 2, 3, 4, 5, or 6 modified nucleotides of the first nucleotide sequence are 2′-fluoro nucleotides. In some embodiments, at least 1 modified nucleotide of the first nucleotide sequence is a 2′-fluoro nucleotide. In some embodiments, at least 2 modified nucleotides of the first nucleotide sequence are 2′-fluoro nucleotides. In some embodiments, at least 3 modified nucleotides of the first nucleotide sequence are 2′-fluoro nucleotides. In some embodiments, at least 4 modified nucleotides of the first nucleotide sequence are 2′-fluoro nucleotides. In some embodiments, at least 5 modified nucleotides of the first nucleotide sequence are 2′-fluoro nucleotides. In some embodiments, at least 6 modified nucleotides of the first nucleotide sequence are 2′-fluoro nucleotides. In some embodiments, 10, 9, 8, 7, 6, 5, 4, 3 or fewer modified nucleotides of the first nucleotide sequence are 2′-fluoro nucleotides. In some embodiments, 10 or fewer modified nucleotides of the first nucleotide sequence are 2′-fluoro nucleotides. In some embodiments, 7 or fewer modified nucleotides of the first nucleotide sequence are 2′-fluoro nucleotides. In some embodiments, 6 or fewer modified nucleotides of the first nucleotide sequence are 2′-fluoro nucleotides. In some embodiments, 5 or fewer modified nucleotides of the first nucleotide sequence are 2′-fluoro nucleotides. In some embodiments, 4 or fewer modified nucleotides of the first nucleotide sequence are 2′-fluoro nucleotides. In some embodiments, 3 or fewer modified nucleotides of the first nucleotide sequence are 2′-fluoro nucleotides. In some embodiments, 2 or fewer modified nucleotides of the first nucleotide sequence are 2′-fluoro nucleotides. In some embodiments, at least one modified nucleotide of the first nucleotide sequence is a 2′-fluoro pyrimidine. In some embodiments, 1, 2, 3, 4, 5, or 6 modified nucleotides of the first nucleotide sequence are 2′-fluoro pyrimidines. In some embodiments, at least one modified nucleotide of the first nucleotide sequence is a 2′-fluoro purine. In some embodiments, 1, 2, 3, 4, 5, or 6 modified nucleotides of the first nucleotide sequence are 2′-fluoro purines. In some embodiments, the 2′-fluoro nucleotide is a 2′-fluoro nucleotide mimic.

In some embodiments, the nucleotide at position 3, 5, 7, 8, 9, 10, 11, 12, 14, 17, and/or 19 from the 5′ end of the first nucleotide sequence is a 2′-fluoro nucleotide. In some embodiments, at least two nucleotides at positions 3, 5, 7, 8, 9, 10, 11, 12, 14, 17, and/or 19 from the 5′ end of the first nucleotide sequence are 2′-fluoro nucleotides. In some embodiments, at least three nucleotides at positions 3, 5, 7, 8, 9, 10, 11, 12, 14, 17, and/or 19 from the 5′ end of the first nucleotide sequence are 2′-fluoro nucleotides. In some embodiments, at least four nucleotides at positions 3, 5, 7, 8, 9, 10, 11, 12, 14, 17, and/or 19 from the 5′ end of the first nucleotide sequence are 2′-fluoro nucleotides. In some embodiments, at least five nucleotides at positions 3, 5, 7, 8, 9, 10, 11, 12, 14, 17, and/or 19 from the 5′ end of the first nucleotide sequence are 2′-fluoro nucleotides. In some embodiments, the nucleotides at positions 3, 5, 7, 8, 9, 10, 11, 12, 14, 17, and/or 19 from the 5′ end of the first nucleotide sequence are 2′-fluoro nucleotides. In some embodiments, the nucleotide at position 3 from the 5′ end of the first nucleotide sequence is a 2′-fluoro nucleotide. In some embodiments, the nucleotide at position 7 from the 5′ end of the first nucleotide sequence is a 2′-fluoro nucleotide. In some embodiments, the nucleotide at position 8 from the 5′ end of the first nucleotide sequence is a 2′-fluoro nucleotide. In some embodiments, the nucleotide at position 9 from the 5′ end of the first nucleotide sequence is a 2′-fluoro nucleotide. In some embodiments, the nucleotide at position 12 from the 5′ end of the first nucleotide sequence is a 2′-fluoro nucleotide. In some embodiments, the nucleotide at position 17 from the 5′ end of the first nucleotide sequence is a 2′-fluoro nucleotide. In some embodiments, the 2′-fluoro nucleotide is a 2′-fluoro nucleotide mimic.

In some embodiments, at least 1, 2, 3, 4, 5, 6, or 7 nucleotides at position 3, 5, 7, 8, 9, 10, 11, 12, 14, 17, and/or 19 from the 5′ end of the first nucleotide sequence is a 2′-fluoro nucleotide. In some embodiments, the nucleotide at positions 3, 5, 7, 8, 9, 10, 11, 12, 14, 17, and/or 19 from the 5′ end of the first nucleotide sequence is a 2′-fluoro nucleotide. In some embodiments, at least two nucleotides at positions 3, 5, 7, 8, 9, 10, 11, 12, 14, 17, and/or 19 from the 5′ end of the first nucleotide sequence are 2′-fluoro nucleotides. In some embodiments, at least three nucleotides at positions 3, 5, 7, 8, 9, 10, 11, 12, 14, 17, and/or 19 from the 5′ end of the first nucleotide sequence are 2′-fluoro nucleotides. In some embodiments, the nucleotides at positions 3, 5, 7, 8, 9, 10, 11, 12, 14, 17, and/or 19 from the 5′ end of the first nucleotide sequence are 2′-fluoro nucleotides. In some embodiments, the nucleotide at position 3 from the 5′ end of the first nucleotide sequence is a 2′-fluoro nucleotide. In some embodiments, the nucleotide at position 5 from the 5′ end of the first nucleotide sequence is a 2′-fluoro nucleotide. In some embodiments, the nucleotide at position 7 from the 5′ end of the first nucleotide sequence is a 2′-fluoro nucleotide. In some embodiments, the nucleotide at position 8 from the 5′ end of the first nucleotide sequence is a 2′-fluoro nucleotide. In some embodiments, the nucleotide at position 9 from the 5′ end of the first nucleotide sequence is a 2′-fluoro nucleotide. In some embodiments, the nucleotide at position 10 from the 5′ end of the first nucleotide sequence is a 2′-fluoro nucleotide. In some embodiments, the nucleotide at position 11 from the 5′ end of the first nucleotide sequence is a 2′-fluoro nucleotide. In some embodiments, the nucleotide at position 12 from the 5′ end of the first nucleotide sequence is a 2′-fluoro nucleotide. In some embodiments, the nucleotide at position 14 from the 5′ end of the first nucleotide sequence is a 2′-fluoro nucleotide. In some embodiments, the nucleotide at position 17 from the 5′ end of the first nucleotide sequence is a 2′-fluoro nucleotide. In some embodiments, the nucleotide at position 19 from the 5′ end of the first nucleotide sequence is a 2′-fluoro nucleotide. In some embodiments, the nucleotide at position 3, 7, 8, 9, 12, and/or 17 from the 5′ end of the first nucleotide sequence is a 2′-fluoro nucleotide. In some embodiments, the nucleotide at position 3, 7, 8, and/or 17 from the 5′ end of the first nucleotide sequence is a 2′-fluoro nucleotide. In some embodiments, the nucleotide at position 3, 7, 8, 9, 12, and/or 17 from the 5′ end of the first nucleotide sequence is a 2′-fluoro nucleotide. In some embodiments, the nucleotide at position 5, 7, 8, and/or 9 from the 5′ end of the first nucleotide sequence is a 2′-fluoro nucleotide. In some embodiments, the nucleotide at position 5, 9, 10, 11, 12, and/or 19 from the 5′ end of the first nucleotide sequence is a 2′-fluoro nucleotide. In some embodiments, the 2′-fluoro nucleotide is a 2′-fluoro nucleotide mimic.

In some embodiments, the 2′-fluoro or 2′-O-methyl nucleotide mimic is a nucleotide mimic of Formula (V):

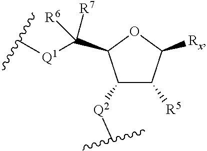

wherein Rx is independently a nucleobase, aryl, heteroaryl, or H, Q¹ and Q² are independently S or O, R⁵ is independently —OCD₃, —F, or —OCH₃, and R⁶ and R⁷ are independently H, D, or CD₃. In some embodiments, the nucleobase is selected from thymine, cytosine, guanine, adenine, uracil, and an analogue or derivative thereof.

In some embodiments, the 2′-fluoro or 2′-O-methyl nucleotide mimic is a nucleotide mimic of Formula (16) -Formula (20):

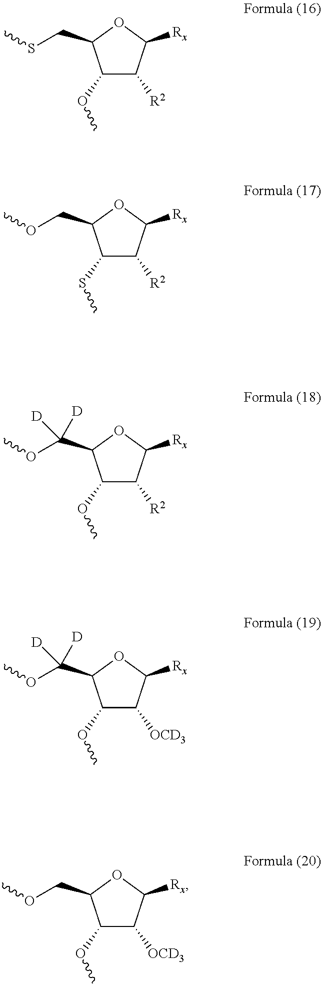

wherein Rₓ is independently a nucleobase, aryl, heteroaryl, or H and R² is F or —OCH₃. In some embodiments, the nucleobase is selected from thymine, cytosine, guanine, adenine, uracil, and an analogue or derivative thereof.

In some embodiments, the sense strand, the antisense strand, or both may each independently comprise at least 1, at least 2, at least 3, at least 4, or at least 5 or more modified nucleotide(s) having the following chemical structure:

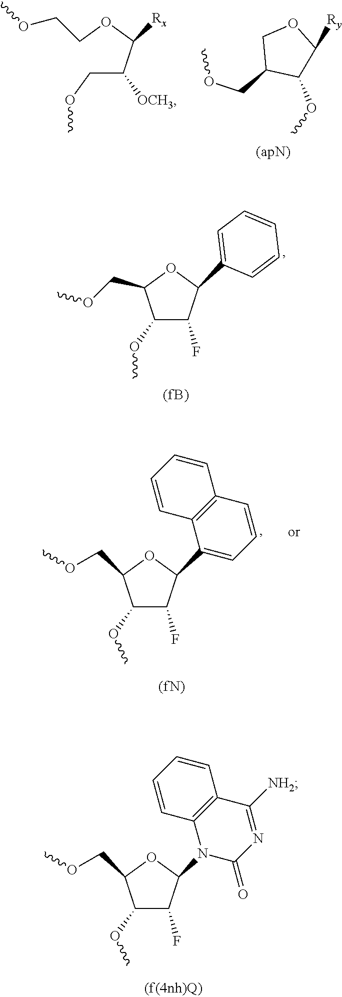

wherein R_{y} is a nucleobase and wherein Rₓ is a nucleobase, aryl, heteroaryl, or H. In some embodiments, the nucleobase is selected from thymine, cytosine, guanine, adenine, uracil, and an analogue or derivative thereof.

In some embodiments, the sense strand, the antisense strand, or both may each independently comprise at least 1, at least 2, at least 3, at least 4, or at least 5 or more modified nucleotide(s) having the following chemical structure:

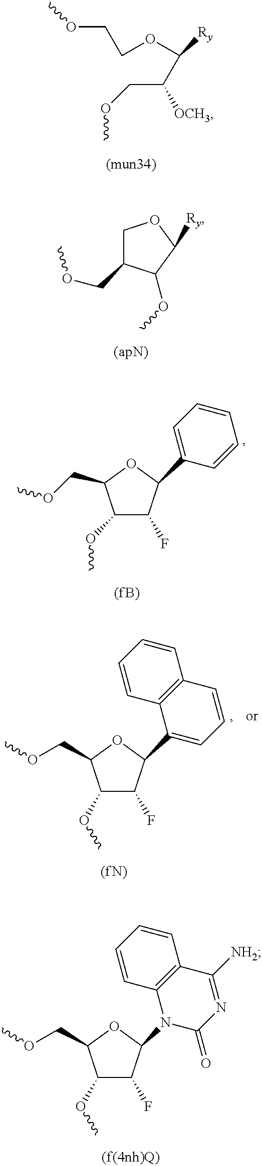

wherein R_{y} is a nucleobase. In some embodiments, the nucleobase is selected from thymine, cytosine, guanine, adenine, uracil, and an analogue or derivative thereof.

For the purposes of the present disclosure, the modified nucleotide may be in any position of the sense strand. In some embodiments, the modified nucleotide may be at position 1, 2, 3, 4, 5, 6, 7, 8, 9, 10, 11, 12, 13, 14, 15, 16, 17, 18, 19, 20, 21, 22, or 23 of the sense strand relative to the 5′ end. For example, when the modified nucleotide is

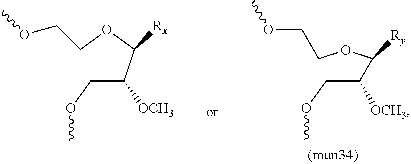

it may be located at position 1, 2, 3, 4, 5, 6, 7, 8, 9, 10, 11, 12, 13, 14, 15, 16, 17, 18, 19, 20, 21, 22, or 23 of the sense strand relative to the 5′ end. In some embodiments, when the modified nucleotide is

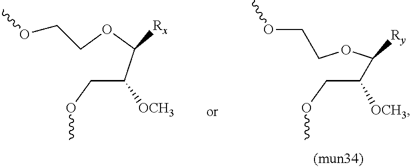

it may be located at position 3, 16, 17, or 18, relative to the 5′ end of the sense strand.

In some embodiments, the first nucleotide sequence comprises, consists of, or consists essentially of ribonucleic acids (RNAs). In some embodiments, the first nucleotide sequence comprises, consists of, or consists essentially of modified RNAs. In some embodiments, the modified RNAs are selected from a 2′-O-methyl RNA and 2′-fluoro RNA. In some embodiments, 15, 16, 17, 18, 19, 20, 21, 22, or 23 modified nucleotides of the first nucleotide sequence are independently selected from 2′-O-methyl RNA and 2′-fluoro RNA.

In some embodiments, the sense strand may further comprise one or more internucleoside linkages independently selected from a phosphodiester (PO) internucleoside linkage, phosphorothioate (PS) internucleoside linkage, mesyl phosphoramidate internucleoside linkage (Ms), phosphorodithioate internucleoside linkage, and PS-mimic internucleoside linkage. In some embodiments, the PS-mimic internucleoside linkage is a sulfo internucleoside linkage.

In some embodiments, the sense strand may further comprise at least 1, 2, 3, 4, 5, 6, 7, 8, 9, 10, 11, 12, 13, 14, or 15 or more phosphorothioate internucleoside linkages. In some embodiments, the sense strand comprises 20, 19, 18, 17, 16, 15, 14, 13, 12, 11, 10, 9, 8, 7, 6, 5, 4, or 3 or fewer phosphorothioate internucleoside linkages. In some embodiments, the sense strand comprises 2 to 10, 2 to 8, 2 to 6, 1 to 5, 1 to 4, 1 to 3, or 1 to 2 phosphorothioate internucleoside linkages. In some embodiments, the sense strand comprises 1 to 2 phosphorothioate internucleoside linkages. In some embodiments, the sense strand comprises 2 to 4 phosphorothioate internucleoside linkages. In some embodiments, at least one phosphorothioate internucleoside linkage is between the nucleotides at positions 1 and 2 from the 5′ end of the first nucleotide sequence. In some embodiments, at least one phosphorothioate internucleoside linkage is between the nucleotides at positions 2 and 3 from the 5′ end of the first nucleotide sequence. In some embodiments, the sense strand comprises two phosphorothioate internucleoside linkages between the nucleotides at positions 1 to 3 from the 5′ end of the first nucleotide sequence.

In some embodiments, the sense strand may further comprise at least 1, 2, 3, 4, 5, 6, 7, 8, 9, 10, 11, 12, 13, 14, or 15 or more mesyl phosphoramidate internucleoside linkages. In some embodiments, the sense strand comprises 20, 19, 18, 17, 16, 15, 14, 13, 12, 11, 10, 9, 8, 7, 6, 5, 4, or 3 or fewer mesyl phosphoramidate internucleoside linkages. In some embodiments, the sense strand comprises 2 to 10, 2 to 8, 2 to 6, 1 to 5, 1 to 4, 1 to 3, or 1 to 2 mesyl phosphoramidate internucleoside linkages. In some embodiments, the sense strand comprises 1 to 2 mesyl phosphoramidate internucleoside linkages. In some embodiments, the sense strand comprises 2 to 4 mesyl phosphoramidate internucleoside linkages.

In some embodiments, the sense strand may comprise any of the modified nucleotides disclosed in the sub-section titled “Modified Nucleotides” below. In some embodiments, the sense strand may comprise a 5′-stabilized end cap, and the 5′-stabilized end cap may be selected from those disclosed in the sub-section titled “5′-Stabilized End Cap” below.

siNA Antisense Strand

Any of the siNA molecules described herein may comprise an antisense strand. The antisense strand may comprise a second nucleotide sequence. The second nucleotide sequence may be 15 to 30, 15 to 25, 15 to 23, 17 to 23, 19 to 23, or 19 to 21 nucleotides in length. In some embodiments, the second nucleotide sequence is 15, 16, 17, 18, 19, 20, 21, 22, 23, 24, 25, 26, 27, 28, 29, or 30 nucleotides in length. In some embodiments, the second nucleotide sequence is at least 19 nucleotides in length. In some embodiments, the second nucleotide sequence is at least 21 nucleotides in length.

In some embodiments, the antisense strand is the same length as the second nucleotide sequence. In some embodiments, the antisense strand is longer than the second nucleotide sequence. In some embodiments, the antisense strand may further comprise 1, 2, 3, 4, or 5 or more nucleotides than the second nucleotide sequence. In some embodiments, the antisense strand is the same length as the sense strand. In some embodiments, the antisense strand is longer than the sense strand. In some embodiments, the antisense strand may further comprise 1, 2, 3, 4, or 5 or more nucleotides than the sense strand. In some embodiments, the antisense strand may further comprise a deoxyribonucleic acid (DNA). In some embodiments, the DNA is thymine (T). In some embodiments, the antisense strand may further comprise a TT sequence. In some embodiments, the antisense strand may further comprise one or more modified nucleotides that are adjacent to the second nucleotide sequence. In some embodiments, the one or more modified nucleotides are independently selected from any of the modified nucleotides disclosed herein (e.g., 2′-fluoro nucleotide, 2′-O-methyl nucleotide, 2′-fluoro nucleotide mimic, 2′-O-methyl nucleotide mimic, or a nucleotide comprising a modified nucleobase).

In some embodiments, the second nucleotide sequence comprises 15, 16, 17, 18, 19, 20, 21, 22, 23, or more modified nucleotides independently selected from a 2′-O-methyl nucleotide and a 2′-fluoro nucleotide. In some embodiments, 70%, 75%, 80%, 85%, 90%, 95% or 100% of the nucleotides in the second nucleotide sequence are modified nucleotides independently selected from a 2′-O-methyl nucleotide and a 2′-fluoro nucleotide. In some embodiments, 100% of the nucleotides in the second nucleotide sequence are modified nucleotides independently selected from a 2′-O-methyl nucleotide and a 2′-fluoro nucleotide.

In some embodiments, between about 15 to 30, 15 to 25, 15 to 24, 15 to 23, 15 to 22, 15 to 21, 17 to 30, 17 to 25, 17 to 24, 17 to 23, 17 to 22, 17 to 21, 18 to 30, 18 to 25, 18 to 24, 18 to 23, 18 to 22, 18 to 21, 19 to 30, 19 to 25, 19 to 24, 19 to 23, 19 to 22, 19 to 21, 20 to 25, 20 to 24, 20 to 23, 21 to 25, 21 to 24, or 21 to 23 modified nucleotides of the second nucleotide sequence are 2′-O-methyl nucleotides. In some embodiments, between about 2 to 20 modified nucleotides of the second nucleotide sequence are 2′-O-methyl nucleotides. In some embodiments, between about 5 to 25 modified nucleotides of the second nucleotide sequence are 2′-O-methyl nucleotides. In some embodiments, between about 10 to 25 modified nucleotides of the second nucleotide sequence are 2′-O-methyl nucleotides. In some embodiments, between about 12 to 25 modified nucleotides of the second nucleotide sequence are 2′-O-methyl nucleotides. In some embodiments, at least 10, 11, 12, 13, 14, 15, 16, 17, 18, 19, 20, 21, or 22 modified nucleotides of the second nucleotide sequence are 2′-O-methyl nucleotides. In some embodiments, at least about 12 modified nucleotides of the second nucleotide sequence are 2′-O-methyl nucleotides. In some embodiments, at least about 13 modified nucleotides of the second nucleotide sequence are 2′-O-methyl nucleotides. In some embodiments, at least about 14 modified nucleotides of the second nucleotide sequence are 2′-O-methyl nucleotides. In some embodiments, at least about 15 modified nucleotides of the second nucleotide sequence are 2′-O-methyl nucleotides. In some embodiments, at least about 16 modified nucleotides of the second nucleotide sequence are 2′-O-methyl nucleotides. In some embodiments, at least about 17 modified nucleotides of the second nucleotide sequence are 2′-O-methyl nucleotides. In some embodiments, at least about 18 modified nucleotides of the second nucleotide sequence are 2′-O-methyl nucleotides. In some embodiments, at least about 19 modified nucleotides of the second nucleotide sequence are 2′-O-methyl nucleotides. In some embodiments, less than or equal to 25, 24, 23, 22, 21, 20, 19, 18, 17, 16, 15, 14, 13, 12, 11, 10, 9, 8, 7, 6, 5, 4, 3, or 2 modified nucleotides of the second nucleotide sequence are 2′-O-methyl nucleotides. In some embodiments, less than or equal to 21 modified nucleotides of the second nucleotide sequence are 2′-O-methyl nucleotides. In some embodiments, less than or equal to 20 modified nucleotides of the second nucleotide sequence are 2′-O-methyl nucleotides. In some embodiments, less than or equal to 19 modified nucleotides of the second nucleotide sequence are 2′-O-methyl nucleotides. In some embodiments, less than or equal to 18 modified nucleotides of the second nucleotide sequence are 2′-O-methyl nucleotides. In some embodiments, less than or equal to 17 modified nucleotides of the second nucleotide sequence are 2′-O-methyl nucleotides. In some embodiments, less than or equal to 16 modified nucleotides of the second nucleotide sequence are 2′-O-methyl nucleotides. In some embodiments, less than or equal to 15 modified nucleotides of the second nucleotide sequence are 2′-O-methyl nucleotides. In some embodiments, less than or equal to 14 modified nucleotides of the second nucleotide sequence are 2′-O-methyl nucleotides. In some embodiments, less than or equal to 13 modified nucleotides of the second nucleotide sequence are 2′-O-methyl nucleotides. In some embodiments, at least one modified nucleotide of the second nucleotide sequence is a 2′-O-methyl pyrimidine. In some embodiments, at least 5, 6, 7, 8, 9, or 10 modified nucleotides of the second nucleotide sequence are 2′-O-methyl pyrimidines. In some embodiments, at least one modified nucleotide of the second nucleotide sequence is a 2′-O-methyl purine. In some embodiments, at least 5, 6, 7, 8, 9, or 10 modified nucleotides of the second nucleotide sequence are 2′-O-methyl purines. In some embodiments, the 2′-O-methyl nucleotide is a 2′-O-methyl nucleotide mimic.

In some embodiments, between 2 to 15 modified nucleotides of the second nucleotide sequence are 2′-fluoro nucleotides. In some embodiments, between 2 to 10 modified nucleotides of the second nucleotide sequence are 2′-fluoro nucleotides. In some embodiments, between 2 to 6 modified nucleotides of the second nucleotide sequence are 2′-fluoro nucleotides. In some embodiments, 1 to 6, 1 to 5, 1 to 4, or 1 to 3 modified nucleotides of the second nucleotide sequence are 2′-fluoro nucleotides. In some embodiments, at least 1, 2, 3, 4, 5, or 6 modified nucleotides of the second nucleotide sequence are 2′-fluoro nucleotides. In some embodiments, at least 1 modified nucleotide of the second nucleotide sequence is a 2′-fluoro nucleotide. In some embodiments, at least 2 modified nucleotides of the second nucleotide sequence are 2′-fluoro nucleotides. In some embodiments, at least 3 modified nucleotides of the second nucleotide sequence are 2′-fluoro nucleotides. In some embodiments, at least 4 modified nucleotides of the second nucleotide sequence are 2′-fluoro nucleotides. In some embodiments, at least 5 modified nucleotides of the second nucleotide sequence are 2′-fluoro nucleotides. In some embodiments, 10, 9, 8, 7, 6, 5, 4, 3 or fewer modified nucleotides of the second nucleotide sequence are 2′-fluoro nucleotides. In some embodiments, 10 or fewer modified nucleotides of the second nucleotide sequence are 2′-fluoro nucleotides. In some embodiments, 7 or fewer modified nucleotides of the second nucleotide sequence are 2′-fluoro nucleotides. In some embodiments, 6 or fewer modified nucleotides of the second nucleotide sequence are 2′-fluoro nucleotides. In some embodiments, 5 or fewer modified nucleotides of the second nucleotide sequence are 2′-fluoro nucleotides. In some embodiments, 4 or fewer modified nucleotides of the second nucleotide sequence are 2′-fluoro nucleotides. In some embodiments, 3 or fewer modified nucleotides of the second nucleotide sequence are 2′-fluoro nucleotides. In some embodiments, 2 or fewer modified nucleotides of the second nucleotide sequence are 2′-fluoro nucleotides. In some embodiments, at least one modified nucleotide of the second nucleotide sequence is a 2′-fluoro pyrimidine. In some embodiments, 1, 2, 3, 4, 5, or 6 modified nucleotides of the second nucleotide sequence are 2′-fluoro pyrimidines. In some embodiments, at least one modified nucleotide of the second nucleotide sequence is a 2′-fluoro purine. In some embodiments, 1, 2, 3, 4, 5, or 6 modified nucleotides of the second nucleotide sequence are 2′-fluoro purines. In some embodiments, the 2′-fluoro nucleotide is a 2′-fluoro nucleotide mimic.

In some embodiments, the 2′-fluoro nucleotide or 2′-O-methyl nucleotide is a 2′-fluoro or 2′-O-methyl nucleotide mimic. In some embodiments, the 2′-fluoro or 2′-O-methyl nucleotide mimic is a nucleotide mimic of Formula (V):

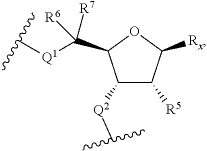

independently a nucleobase, aryl, heteroaryl, or H, Q¹ and Q² are independently S or O, R⁵ is independently —OCD₃, —F, or —OCH₃, and R⁶ and R⁷ are independently H, D, or CD₃. In some embodiments, the nucleobase is selected from thymine, cytosine, guanine, adenine, uracil, and an analogue or derivative thereof.

In some embodiments, the 2′-fluoro or 2′-O-methyl nucleotide mimic is a nucleotide mimic of Formula (16) -Formula (20):

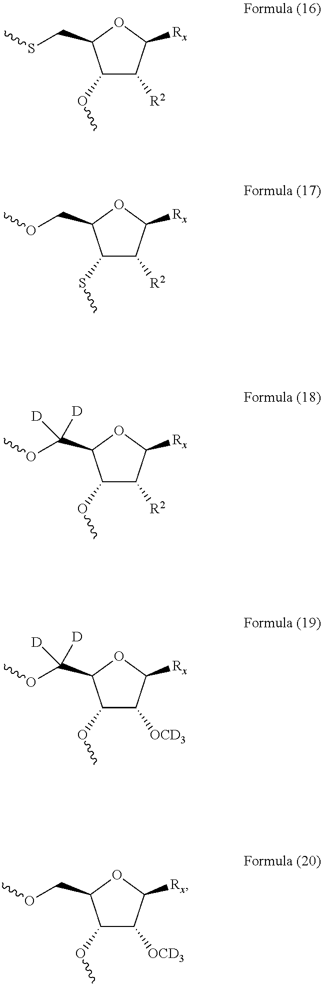

wherein Rₓ is a nucleobase, aryl, heteroaryl, or H and R² is independently F or —OCH₃. In some embodiments, the nucleobase is selected from thymine, cytosine, guanine, adenine, uracil, and an analogue or derivative thereof.

In some embodiments, the antisense strand, sense strand, or both may each independently comprise at least 1, at least 2, at least 3, at least 4, or at least 5 or more modified nucleotide(s) having the following chemical structure:

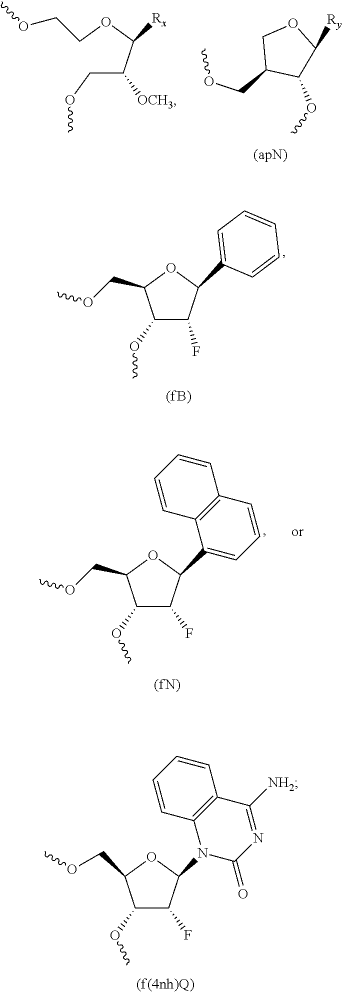

wherein R_{y} is a nucleobase and wherein Rₓ is a nucleobase, aryl, heteroaryl, or H. In some embodiments, the nucleobase is selected from thymine, cytosine, guanine, adenine, uracil, and an analogue or derivative thereof.

In some embodiments, the antisense strand, sense strand, or both may each independently comprise at least 1, at least 2, at least 3, at least 4, or at least 5 or more modified nucleotide(s) having the following chemical structure:

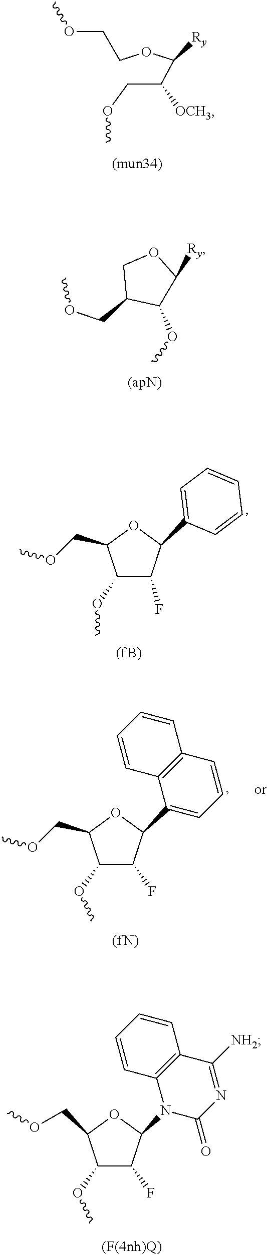

wherein R_{y} is a nucleobase. In some embodiments, the nucleobase is selected from thymine, cytosine, guanine, adenine, uracil, and an analogue or derivative thereof.

For the purposes of the present disclosure, the modified nucleotide may be in any position of the antisense strand. In some embodiments, the modified nucleotide may be at position 1, 2, 3, 4, 5, 6, 7, 8, 9, 10, 11, 12, 13, 14, 15, 16, 17, 18, 19, 20, 21, 22, or 23 of the antisense strand relative to the 5′ end.

In some embodiments, at least 1, 2, 3, 4, 5, 6, 7, 8, or 9 nucleotides at position 2, 5, 6, 8, 10, 14, 16, 17, and/or 18 from the 5′ end of the second nucleotide sequence is a 2′-fluoro nucleotide. In some embodiments, the nucleotide at position 2, 5, 6, 8, 10, 14, 16, 17, and/or 18 from the 5′ end of the second nucleotide sequence is a 2′-fluoro nucleotide. In some embodiments, at least two nucleotides at positions 2, 5, 6, 8, 10, 14, 16, 17, and/or 18 from the 5′ end of the second nucleotide sequence are 2′-fluoro nucleotides. In some embodiments, at least three nucleotides at positions 2, 5, 6, 8, 10, 14, 16, 17, and/or 18 from the 5′ end of the second nucleotide sequence are 2′-fluoro nucleotides. In some embodiments, at least four nucleotides at positions 2, 5, 6, 8, 10, 14, 16, 17, and/or 18 from the 5′ end of the second nucleotide sequence are 2′-fluoro nucleotides. In some embodiments, at least five nucleotides at positions 2, 5, 6, 8, 10, 14, 16, 17, and/or 18 from the 5′ end of the second nucleotide sequence are 2′-fluoro nucleotides. In some embodiments, the nucleotides at positions 2 and/or 14 from the 5′ end of the second nucleotide sequence are 2′-fluoro nucleotides. In some embodiments, the nucleotides at positions 2, 6, and/or 16 from the 5′ end of the second nucleotide sequence are 2′-fluoro nucleotides. In some embodiments, the nucleotides at positions 2, 6, 14, and/or 16 from the 5′ end of the second nucleotide sequence are 2′-fluoro nucleotides. In some embodiments, the nucleotides at positions 2, 6, 10, 14, and/or 18 from the 5′ end of the second nucleotide sequence are 2′-fluoro nucleotides. In some embodiments, the nucleotides at positions 2, 5, 8, 14, and/or 17 from the 5′ end of the second nucleotide sequence are 2′-fluoro nucleotides. In some embodiments, the nucleotide at position 2 from the 5′ end of the second nucleotide sequence is a 2′-fluoro nucleotide. In some embodiments, the nucleotide at position 5 from the 5′ end of the second nucleotide sequence is a 2′-fluoro nucleotide. In some embodiments, the nucleotide at position 6 from the 5′ end of the second nucleotide sequence is a 2′-fluoro nucleotide. In some embodiments, the nucleotide at position 8 from the 5′ end of the second nucleotide sequence is a 2′-fluoro nucleotide. In some embodiments, the nucleotide at position 10 from the 5′ end of the second nucleotide sequence is a 2′-fluoro nucleotide. In some embodiments, the nucleotide at position 14 from the 5′ end of the second nucleotide sequence is a 2′-fluoro nucleotide. In some embodiments, the nucleotide at position 16 from the 5′ end of the second nucleotide sequence is a 2′-fluoro nucleotide. In some embodiments, the nucleotide at position 17 from the 5′ end of the second nucleotide sequence is a 2′-fluoro nucleotide. In some embodiments, the nucleotide at position 18 from the 5′ end of the second nucleotide sequence is a 2′-fluoro nucleotide. In some embodiments, the 2′-fluoro nucleotide is a 2′-fluoro nucleotide mimic.

In some embodiments, the nucleotides in the second nucleotide sequence are arranged in an alternating 1:3 modification pattern, wherein 1 nucleotide is a 2′-fluoro nucleotide and 3 nucleotides are 2′-O-methyl nucleotides, and wherein the alternating 1:3 modification pattern occurs at least 2 times. In some embodiments, the alternating 1:3 modification pattern occurs 2-5 times. In some embodiments, at least two of the alternating 1:3 modification pattern occur consecutively. In some embodiments, at least two of the alternating 1:3 modification pattern occurs nonconsecutively. In some embodiments, at least 1, 2, 3, 4, or 5 alternating 1:3 modification pattern begins at nucleotide position 2, 6, 10, 14, and/or 18 from the 5′ end of the antisense strand. In some embodiments, at least one alternating 1:3 modification pattern begins at nucleotide position 2 from the 5′ end of the antisense strand. In some embodiments, wherein at least one alternating 1:3 modification pattern begins at nucleotide position 6 from the 5′ end of the antisense strand. In some embodiments, at least one alternating 1:3 modification pattern begins at nucleotide position 10 from the 5′ end of the antisense strand. In some embodiments, at least one alternating 1:3 modification pattern begins at nucleotide position 14 from the 5′ end of the antisense strand. In some embodiments, at least one alternating 1:3 modification pattern begins at nucleotide position 18 from the 5′ end of the antisense strand. In some embodiments, the 2′-fluoro nucleotide is a 2′-fluoro nucleotide mimic.

In some embodiments, the nucleotides in the second nucleotide sequence are arranged in an alternating 1:2 modification pattern, wherein 1 nucleotide is a 2′-fluoro nucleotide and 2 nucleotides are 2′-O-methyl nucleotides, and wherein the alternating 1:2 modification pattern occurs at least 2 times. In some embodiments, the alternating 1:2 modification pattern occurs 2-5 times. In some embodiments, at least two of the alternating 1:2 modification pattern occurs consecutively. In some embodiments, at least two of the alternating 1:2 modification pattern occurs nonconsecutively. In some embodiments, at least 1, 2, 3, 4, or 5 alternating 1:2 modification pattern begins at nucleotide position 2, 5, 8, 14, and/or 17 from the 5′ end of the antisense strand. In some embodiments, at least one alternating 1:2 modification pattern begins at nucleotide position 2 from the 5′ end of the antisense strand. In some embodiments, at least one alternating 1:2 modification pattern begins at nucleotide position 5 from the 5′ end of the antisense strand. In some embodiments, at least one alternating 1:2 modification pattern begins at nucleotide position 8 from the 5′ end of the antisense strand. In some embodiments, at least one alternating 1:2 modification pattern begins at nucleotide position 14 from the 5′ end of the antisense strand. In some embodiments, at least one alternating 1:2 modification pattern begins at nucleotide position 17 from the 5′ end of the antisense strand. In some embodiments, the 2′-fluoro nucleotide is a 2′-fluoro nucleotide mimic.

In some embodiments, the second nucleotide sequence comprises, consists of, or consists essentially of ribonucleic acids (RNAs). In some embodiments, the second nucleotide sequence comprises, consists of, or consists essentially of modified RNAs. In some embodiments, the modified RNAs are selected from a 2′-O-methyl RNA and 2′-fluoro RNA. In some embodiments, 15, 16, 17, 18, 19, 20, 21, 22, or 23 modified nucleotides of the second nucleotide sequence are independently selected from 2′-O-methyl RNA and 2′-fluoro RNA. In some embodiments, the 2′-fluoro nucleotide is a 2′-fluoro nucleotide mimic.

In some embodiments, the sense strand may further comprise one or more internucleoside linkages independently selected from a phosphodiester (PO) internucleoside linkage, phosphorothioate (PS) internucleoside linkage, phosphorodithioate internucleoside linkage, and PS-mimic internucleoside linkage. In some embodiments, the PS-mimic internucleoside linkage is a sulfo internucleoside linkage.

In some embodiments, the antisense strand may further comprise at least 1, 2, 3, 4, 5, 6, 7, 8, 9, 10, 11, 12, 13, 14, or 15 or more phosphorothioate internucleoside linkages. In some embodiments, the antisense strand comprises 20, 19, 18, 17, 16, 15, 14, 13, 12, 11, 10, 9, 8, 7, 6, 5, 4, or 3 or fewer phosphorothioate internucleoside linkages. In some embodiments, the antisense strand comprises 2 to 10, 2 to 8, 2 to 6, 1 to 5, 1 to 4, 1 to 3, or 1 to 2 phosphorothioate internucleoside linkages. In some embodiments, the antisense strand comprises 2 to 10, 2 to 8, 2 to 6, 1 to 5, 1 to 4, 1 to 3, or 1 to 2 phosphorothioate internucleoside linkages. In some embodiments, the antisense strand comprises 2 to 8 phosphorothioate internucleoside linkages. In some embodiments, the antisense strand comprises 3 to 8 phosphorothioate internucleoside linkages. In some embodiments, the antisense strand comprises 4 to 8 phosphorothioate internucleoside linkages. In some embodiments, at least one phosphorothioate internucleoside linkage is between the nucleotides at positions 1 and 2 from the 5′ end of the second nucleotide sequence. In some embodiments, at least one phosphorothioate internucleoside linkage is between the nucleotides at positions 2 and 3 from the 5′ end of the second nucleotide sequence. In some embodiments, at least one phosphorothioate internucleoside linkage is between the nucleotides at positions 1 and 2 from the 3′ end of the second nucleotide sequence. In some embodiments, at least one phosphorothioate internucleoside linkage is between the nucleotides at positions 2 and 3 from the 3′ end of the second nucleotide sequence. In some embodiments, the antisense strand comprises two phosphorothioate internucleoside linkages between the nucleotides at positions 1 to 3 from the 5′ end of the first nucleotide sequence. In some embodiments, the antisense strand comprises two phosphorothioate internucleoside linkages between the nucleotides at positions 1 to 3 from the 3′ end of the first nucleotide sequence. In some embodiments, the antisense strand comprises (a) two phosphorothioate internucleoside linkages between the nucleotides at positions 1 to 3 from the 5′ end of the first nucleotide sequence; and (b) two phosphorothioate internucleoside linkages between the nucleotides at positions 1 to 3 from the 3′ end of the first nucleotide sequence.

In some embodiments, the antisense strand may further comprise at least 1, 2, 3, 4, 5, 6, 7, 8, 9, 10, 11, 12, 13, 14, or 15 or more mesyl phosphoramidate internucleoside linkages. In some embodiments, the antisense strand comprises 20, 19, 18, 17, 16, 15, 14, 13, 12, 11, 10, 9, 8, 7, 6, 5, 4, or 3 or fewer mesyl phosphoramidate internucleoside linkages. In some embodiments, the antisense strand comprises 2 to 10, 2 to 8, 2 to 6, 1 to 5, 1 to 4, 1 to 3, or 1 to 2 mesyl phosphoramidate internucleoside linkages. In some embodiments, the antisense strand comprises 2 to 10, 2 to 8, 2 to 6, 1 to 5, 1 to 4, 1 to 3, or 1 to 2 mesyl phosphoramidate internucleoside linkages. In some embodiments, the antisense strand comprises 2 to 8 mesyl phosphoramidate internucleoside linkages. In some embodiments, the antisense strand comprises 3 to 8 mesyl phosphoramidate internucleoside linkages. In some embodiments, the antisense strand comprises 4 to 8 mesyl phosphoramidate internucleoside linkages.

In some embodiments, at least one end of the ds-siNA is a blunt end. In some embodiments, at least one end of the ds-siNA comprises an overhang, wherein the overhang comprises at least one nucleotide. In some embodiments, both ends of the ds-siNA comprise an overhang, wherein the overhang comprises at least one nucleotide. In some embodiments, the overhang comprises 1 to 5 nucleotides, 1 to 4 nucleotides, 1 to 3 nucleotides, or 1 to 2 nucleotides. In some embodiments, the overhang consists of 1 to 2 nucleotides.

In some embodiments, the sense strand may comprise any of the modified nucleotides disclosed in the sub-section titled “Modified Nucleotides” below. In some embodiments, the sense strand may comprise a 5′-stabilized end cap, and the 5′-stabilized end cap may be selected from those disclosed in the sub-section titled “5′-Stabilized End Cap” below.

### Modified Nucleotides

The siNA molecules disclosed herein comprise one or more modified nucleotides. In some embodiments, the sense strands disclosed herein comprise one or more modified nucleotides. In some embodiments, any of the first nucleotide sequences disclosed herein comprise one or more modified nucleotides. In some embodiments, the antisense strands disclosed herein comprise one or more modified nucleotides. In some embodiments, any of the second nucleotide sequences disclosed herein comprise one or more modified nucleotides. In some embodiments, the one or more modified nucleotides is adjacent to the first nucleotide sequence. In some embodiments, at least one modified nucleotide is adjacent to the 5′ end of the first nucleotide sequence. In some embodiments, at least one modified nucleotide is adjacent to the 3′ end of the first nucleotide sequence. In some embodiments, at least one modified nucleotide is adjacent to the 5′ end of the first nucleotide sequence and at least one modified nucleotide is adjacent to the 3′ end of the first nucleotide sequence. In some embodiments, the one or more modified nucleotides is adjacent to the second nucleotide sequence. In some embodiments, at least one modified nucleotide is adjacent to the 5′ end of the second nucleotide sequence. In some embodiments, at least one modified nucleotide is adjacent to the 3′ end of the second nucleotide sequence. In some embodiments, at least one modified nucleotide is adjacent to the 5′ end of the second nucleotide sequence and at least one modified nucleotide is adjacent to the 3′ end of the second nucleotide sequence. In some embodiments, a 2′-O-methyl nucleotide in any of sense strands or first nucleotide sequences disclosed herein is replaced with a modified nucleotide. In some embodiments, a 2′-O-methyl nucleotide in any of antisense strands or second nucleotide sequences disclosed herein is replaced with a modified nucleotide.

In some embodiments, any of the siNA molecules, siNAs, sense strands, first nucleotide sequences, antisense strands, and second nucleotide sequences disclosed herein comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more modified nucleotides. In some embodiments, 1%, 2%, 3%, 4%, 5%, 10%, 15%, 20%, 25%, 30%, 35%, 40%, 45%, 50%, 55%, 60%, 70%, 75%, 80%, 85%, 86%, 87%, 88%, 90%, 91%, 92%, 93%, 94%, 95%, 96%, 97%, 98%, 99% or 100% of the nucleotides in the siNA molecule, siNA, sense strand, first nucleotide sequence, antisense strand, or second nucleotide sequence are modified nucleotides.

In some embodiments, a modified nucleotide is selected from the group consisting of 2′-fluoro nucleotide, 2′-O-methyl nucleotide, 2′-fluoro nucleotide mimic, 2′-O-methyl nucleotide mimic, a locked nucleic acid, an unlocked nucleic acid, and a nucleotide comprising a modified nucleobase. In some embodiments, the unlocked nucleic acid is a 2′,3′-unlocked nucleic acid. In some embodiments, the unlocked nucleic acid is a 3′,4′-unlocked nucleic acid (e.g., mun34) in which the furanose ring lacks a bond between the 3′ and 4; carbons.

In some aspects, the siNA of the present disclosure will comprise at least one modified nucleotide selected from:

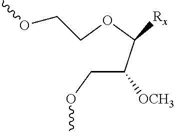

(wherein Rₓ is a nucleobase, aryl, heteroaryl, or H)

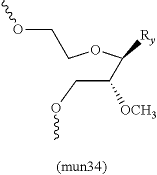

wherein R_{y} is a nucleobase,

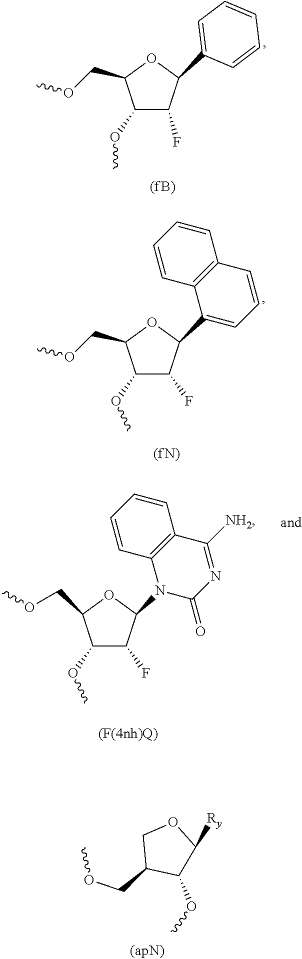

wherein R_{y} is a nucleobase, or combinations thereof. In some embodiments, the siNA may comprise at least 2, at least 3, at least 4, or at least 5 or more of these modified nucleotides. In some embodiments, the sense strand may comprise at least 1, at least 2, at least 3, at least 4, or at least 5 or more of

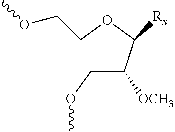

(wherein Rₓ is a nucleobase, aryl, heteroaryl, H),

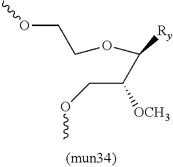

wherein R_{y} is a nucleobase,

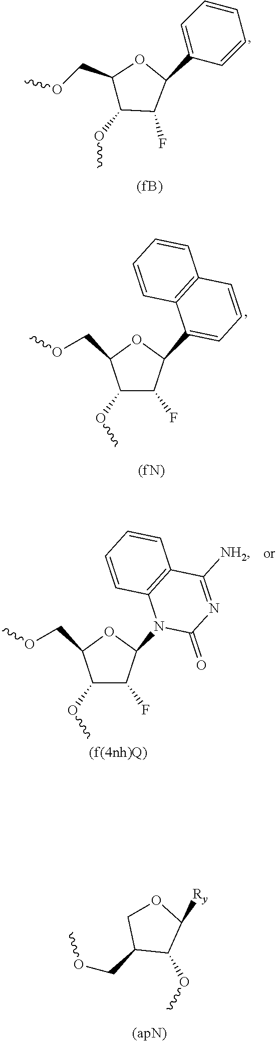

wherein R_{y} is a nucleobase, or combinations thereof. In some embodiments, the antisense strand may comprise at least 1, at least 2, at least 3, at least 4, or at least 5 or more of

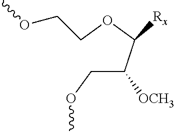

(wherein Rₓ is a nucleobase, aryl, heteroaryl, or H),

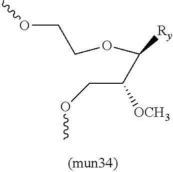

wherein R_{y} is a nucleobase,

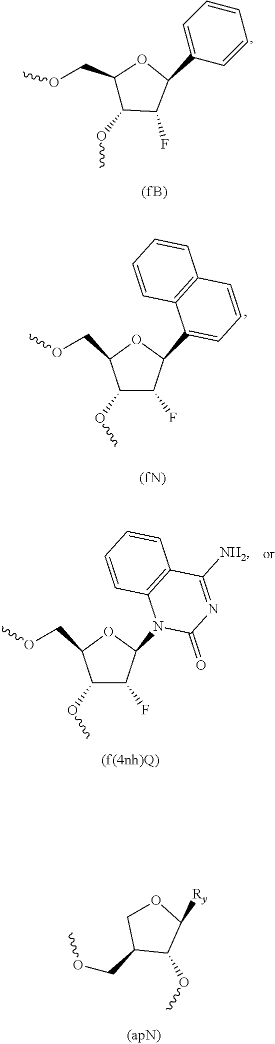

wherein R_{y} is a nucleobase, or combinations thereof. In some embodiments, both the sense strand and the antisense strand may each independently comprise at least 1, at least 2, at least 3, at least 4, or at least 5 or more of

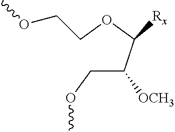

(wherein Rₓ is a nucleobase, aryl, heteroaryl, or H),

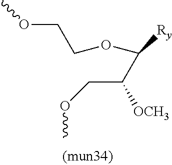

wherein R_{y} is a nucleobase,

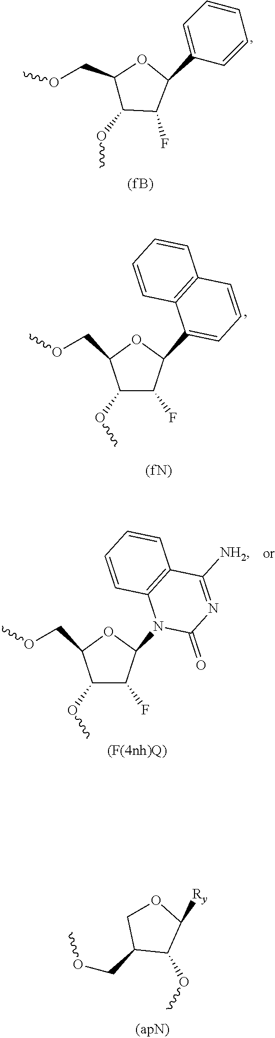

wherein R_{y} is a nucleobase, or combinations thereof. In some embodiments, the nucleobase is selected from thymine, cytosine, guanine, adenine, uracil, and an analogue or derivative thereof. For example, in some embodiments of

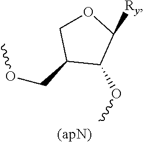

the modified nucleotide may have a structure of

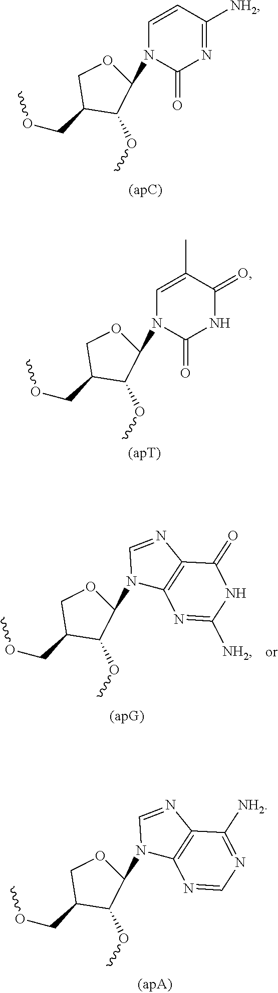

In some embodiments, any of the siRNAs disclosed herein may additionally comprise other modified nucleotides, such as 2′-fluoro or 2′-O-methyl nucleotide mimics. For example, the disclosed siNA may comprise at least 1, 2, 3, 4, 5, 6, 7, 8, 9, or 10 or more 2′-fluoro or 2′-O-methyl nucleotide mimics. In some embodiments, any of the sense strands disclosed herein comprise at least 1, 2, 3, 4, 5, 6, 7, 8, 9, or 10 or more 2′-fluoro or 2′-O-methyl nucleotide mimics. In some embodiments, any of the first nucleotide sequences disclosed herein comprise at least 1, 2, 3, 4, 5, 6, 7, 8, 9, or 10 or more 2′-fluoro or 2′-O-methyl nucleotide mimics. In some embodiments, any of the antisense strand disclosed herein comprise at least 1, 2, 3, 4, 5, 6, 7, 8, 9, or 10 or more 2′-fluoro or 2′-O-methyl nucleotide mimics. In some embodiments, any of the second nucleotide sequences disclosed herein comprise at least 1, 2, 3, 4, 5, 6, 7, 8, 9, or 10 or more 2′-fluoro or 2′-O-methyl nucleotide mimics. In some embodiments, the 2′-fluoro or 2′-O-methyl nucleotide mimic is a nucleotide mimic of Formula (16) -Formula (20):

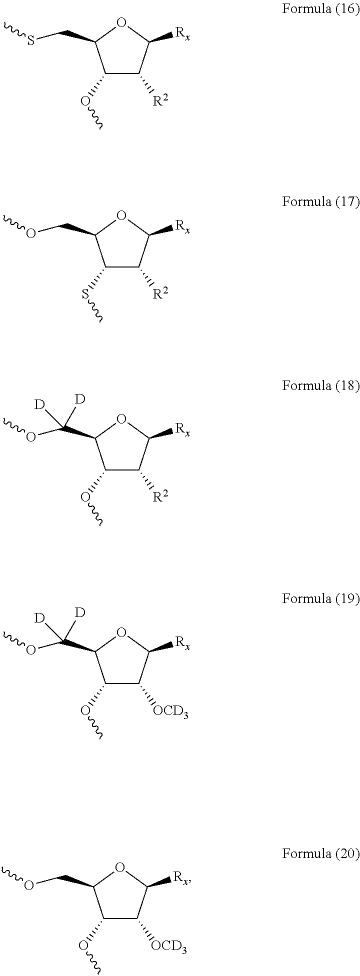

wherein Rₓ is a nucleobase, aryl, heteroaryl, or H and R² is independently F or —OCH₃. In some embodiments, the nucleobase is selected from thymine, cytosine, guanine, adenine, uracil, and an analogue or derivative thereof.

In some embodiments, the siNA molecules disclosed herein comprise at least one 2′-fluoro nucleotide, at least one 2′-O-methyl nucleotide, and at least one 2′-fluoro or 2′-O-methyl nucleotide mimic. In some embodiments, the at least one 2′-fluoro or 2′-O-methyl nucleotide mimic is adjacent to the first nucleotide sequence. In some embodiments, the at least one 2′-fluoro or 2′-O-methyl nucleotide mimic is adjacent to the 5′ end of first nucleotide sequence. In some embodiments, the at least one 2′-fluoro or 2′-O-methyl nucleotide mimic is adjacent to the 3′ end of first nucleotide sequence. In some embodiments, the at least one 2′-fluoro or 2′-O-methyl nucleotide mimic is adjacent to the second nucleotide sequence. In some embodiments, the at least one 2′-fluoro or 2′-O-methyl nucleotide mimic is adjacent to the 5′ end of second nucleotide sequence. In some embodiments, the at least one 2′-fluoro or 2′-O-methyl nucleotide mimic is adjacent to the 3′ end of second nucleotide sequence. In some embodiments, the first nucleotide sequence does not comprise a 2′-fluoro nucleotide mimic. In some embodiments, the first nucleotide sequence does not comprise a 2′-O-methyl nucleotide mimic. In some embodiments, the second nucleotide sequence does not comprise a 2′-fluoro nucleotide mimic. In some embodiments, the second nucleotide sequence does not comprise a 2′-O-methyl nucleotide mimic.

In some embodiments, any of the siRNAs, sense strands, first nucleotide sequences, antisense strands, or second nucleotide sequences disclosed herein comprise at least one modified nucleotide that is

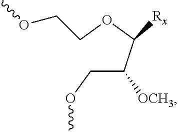

wherein Rₓ is a nucleobase, aryl, heteroaryl, or H; or

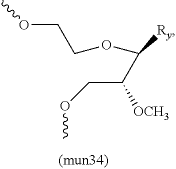

wherein R_{y} is a nucleobase.

### Phosphorylation Blocker

Further disclosed herein are siNA molecules comprising a phosphorylation blocker. In some embodiments, a 2′-O-methyl nucleotide in any of sense strands or first nucleotide sequences disclosed herein is replaced with a nucleotide containing a phosphorylation blocker. In some embodiments, a 2′-O-methyl nucleotide in any of antisense strands or second nucleotide sequences disclosed herein is replaced with a nucleotide containing a phosphorylation blocker. In some embodiments, a 2′-O-methyl nucleotide in any of sense strands or first nucleotide sequences disclosed herein is further modified to contain a phosphorylation blocker. In some embodiments, a 2′-O-methyl nucleotide in any of antisense strands or second nucleotide sequences disclosed herein is further modified to contain a phosphorylation blocker.

In some embodiments, any of the siNA molecules disclosed herein comprise a phosphorylation blocker of Formula (IV):

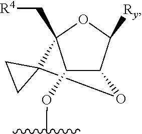

wherein R_{y} is a nucleobase, R⁴ is —O—R³⁰ or —NR³¹R³², R³⁰ is C₁-C₈ substituted or unsubstituted alkyl; and R³¹ and R³² together with the nitrogen to which they are attached form a substituted or unsubstituted heterocyclic ring. In some embodiments, the nucleobase is selected from thymine, cytosine, guanine, adenine, uracil, and an analogue or derivative thereof.

In some embodiments, any of the siNA molecules disclosed herein comprise a phosphorylation blocker of Formula (IV):

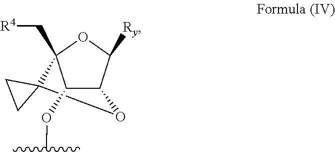

wherein R_{y} is a nucleobase, and R⁴ is —OCH₃ or —N(CH₂CH₂)₂O. In some embodiments, the nucleobase is selected from thymine, cytosine, guanine, adenine, uracil, and an analogue or derivative thereof.

In some embodiments, a siNA molecule comprises (a) a phosphorylation blocker of Formula (IV):

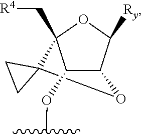

wherein R_{y} is a nucleobase, R⁴ is —O—R³⁰ or —NR³¹R³², R³⁰ is C₁-C₈ substituted or unsubstituted alkyl; and R³¹ and R³² together with the nitrogen to which they are attached form a substituted or unsubstituted heterocyclic ring; and (b) a short interfering nucleic acid (siNA), wherein the phosphorylation blocker is conjugated to the siNA. In some embodiments, the nucleobase is selected from thymine, cytosine, guanine, adenine, uracil, and an analogue or derivative thereof.

In some embodiments, a siNA molecule comprises (a) a phosphorylation blocker of Formula (IV):

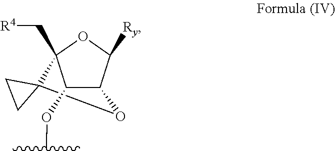

wherein R_{y} is a nucleobase, and R⁴ is —OCH₃ or —N(CH₂CH₂)₂O; and (b) a short interfering nucleic acid (siNA), wherein the phosphorylation blocker is conjugated to the siNA.

In some embodiments, the phosphorylation blocker is attached to the 3′ end of the sense strand or first nucleotide sequence. In some embodiments, the phosphorylation blocker is attached to the 3′ end of the sense strand or first nucleotide sequence via 1, 2, 3, 4, or 5 or more linkers. In some embodiments, the phosphorylation blocker is attached to the 5′ end of the sense strand or first nucleotide sequence. In some embodiments, the phosphorylation blocker is attached to the 5′ end of the sense strand or first nucleotide sequence via 1, 2, 3, 4, or 5 or more linkers. In some embodiments, the phosphorylation blocker is attached to the 3′ end of the antisense strand or second nucleotide sequence. In some embodiments, the phosphorylation blocker is attached to the 3′ end of the antisense strand or second nucleotide sequence via 1, 2, 3, 4, or 5 or more linkers. In some embodiments, the phosphorylation blocker is attached to the 5′ end of the antisense strand or second nucleotide sequence. In some embodiments, the phosphorylation blocker is attached to the 5′ end of the antisense strand or second nucleotide sequence via 1, 2, 3, 4, or 5 or more linkers. In some embodiments, the one or more linkers are independently selected from the group consisting of a phosphodiester linker, phosphorothioate linker, mesyl phosphoramidate linker and phosphorodithioate linker.

### Conjugated Moiety

Further disclosed herein are siNA molecules comprising a conjugated moiety. In some embodiments, the conjugated moiety is selected from galactosamine, peptides, proteins, sterols, lipids, phospholipids biotin, phenoxazines, active drug substance, cholesterols, phenanthridine, anthaquinone, acridine, fluoresceins, rhodamines, coumarins, and dyes. In some embodiments, the conjugated moiety is attached to the 3′ end of the sense strand or first nucleotide sequence. In some embodiments, the conjugated moiety is attached to the 3′ end of the sense strand or first nucleotide sequence via 1, 2, 3, 4, or 5 or more linkers. In some embodiments, the conjugated moiety is attached to the 5′ end of the sense strand or first nucleotide sequence. In some embodiments, the conjugated moiety is attached to the 5′ end of the sense strand or first nucleotide sequence via 1, 2, 3, 4, or 5 or more linkers. In some embodiments, the conjugated moiety is attached to the 3′ end of the antisense strand or second nucleotide sequence. In some embodiments, the conjugated moiety is attached to the 3′ end of the antisense strand or second nucleotide sequence via 1, 2, 3, 4, or 5 or more linkers. In some embodiments, the conjugated moiety is attached to the 5′ end of the antisense strand or second nucleotide sequence. In some embodiments, the conjugated moiety is attached to the 5′ end of the antisense strand or second nucleotide sequence via 1, 2, 3, 4, or 5 or more linkers. In some embodiments, the one or more linkers are independently selected from the group consisting of a phosphodiester linker, phosphorothioate linker, phosphorodithioate linker, and mesyl phosphoramidate linker.

In some embodiments, the conjugated moiety is galactosamine. In some embodiments, any of the siNAs disclosed herein are attached to a conjugated moiety that is galactosamine In some embodiments, the galactosamine is N-acetylgalactosamine (GalNAc). In some embodiments, any of the siNA molecules disclosed herein comprise GalNAc. In some embodiments, the GalNAc is of Formula (VI):

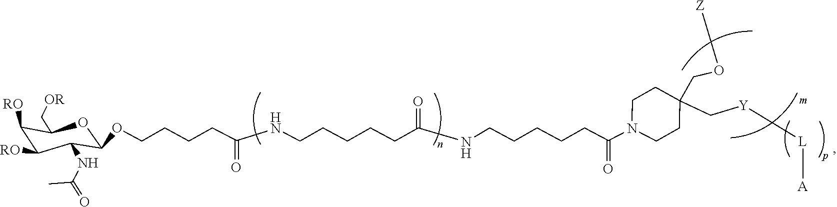

wherein m is 1, 2, 3, 4, or 5; each n is independently 1 or 2; p is 0 or 1; each R is independently H or a first protecting group; each Y is independently selected from —O—P(═O)(SH)—, —O—P(═O)(O)—, —O—P(═O)(OH)—, —O—P(S)S—, and —O—; Z is H or a second protecting group; either L is a linker or L and Y in combination are a linker; and A is H, OH, a third protecting group, an activated group, or an oligonucleotide. In some embodiments, the first protecting group is acetyl. In some embodiments, the second protecting group is trimethoxytrityl (TMT). In some embodiments, the activated group is a phosphoramidite group. In some embodiments, the phosphoramidite group is a cyanoethoxy N,N-diisopropylphosphoramidite group. In some embodiments, the linker is a C₆—NH₂ group. In some embodiments, A is a short interfering nucleic acid (siNA) or siNA molecule. In some embodiments, m is 3. In some embodiments, R is H, Z is H, and n is 1. In some embodiments, R is H, Z is H, and n is 2.

In some embodiments, the GalNAc is Formula (VII):

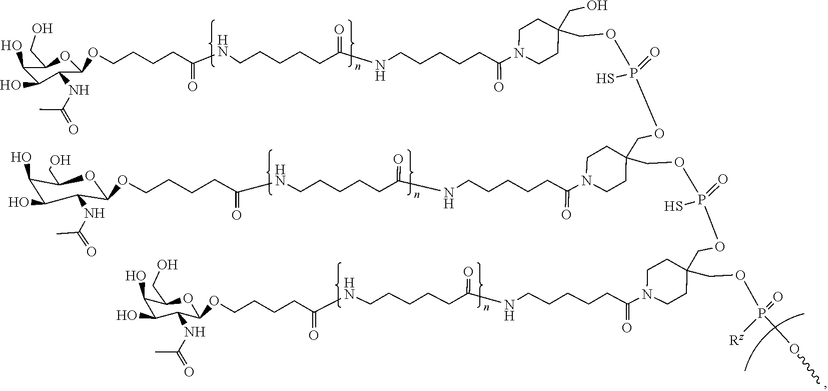

wherein R^{z} is OH or SH; and each n is independently 1 or 2. In some embodiments, the targeting ligand may be a GalNAc targeting ligand may comprise 1, 2, 3, 4, 5 or 6 GalNAc units. In some embodiments, the targeting ligand may be a GalNAc selected from GalNAc2, GalNAc3, GalNAc4 (the GalNAc of Formula VII, wherein n=1 and R^{z}=OH), GalNAc5, and GalNAc6.

In some embodiments, the GalNAc may be GalNAc amidite (i.e., compound 40-9, see Example 22), GalNAc 4 CPG (i.e., compound 40-8, see Example 22 and Example 23), GalNAc phophoramidite, or GalNAc4-ps-GalNAc4-ps-GalNAc4. These GalNAc moieties are shown below:

GalNAc 4 moieties
GalNAc4 phosphoramidite 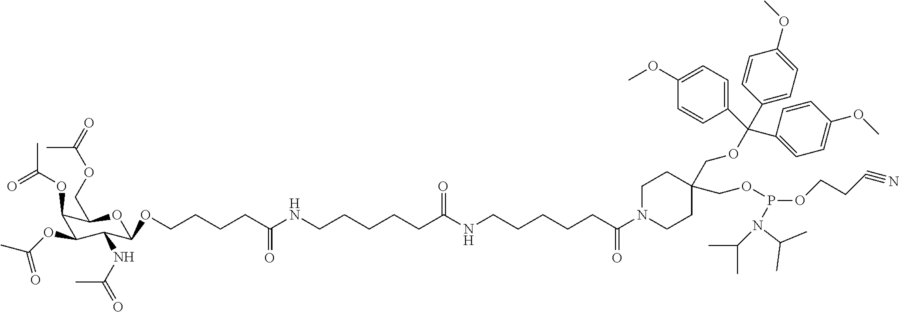
GalNAc4 CPG

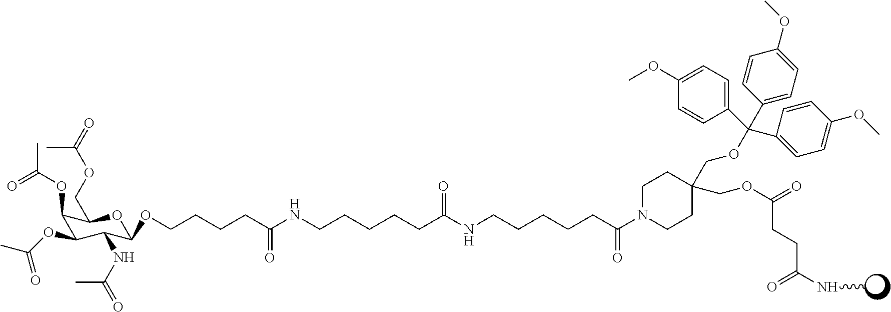

GalNAc3, GalNAc4, GalNAc5 and GalNAc6 may be conjugated to an siNA disclosed herein during synthesis with 1 2, or 3 moieties. Further GalNAc moieties, such as GalNAcl and GalNAc2, can be used to form 5′ and 3′-GalNAc using post synthesis conjugation.

GalNac Phosphoramidites
GalNAc building blocks

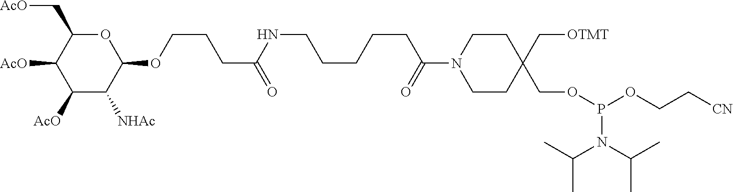

GalNAc-3 phosphoramidite

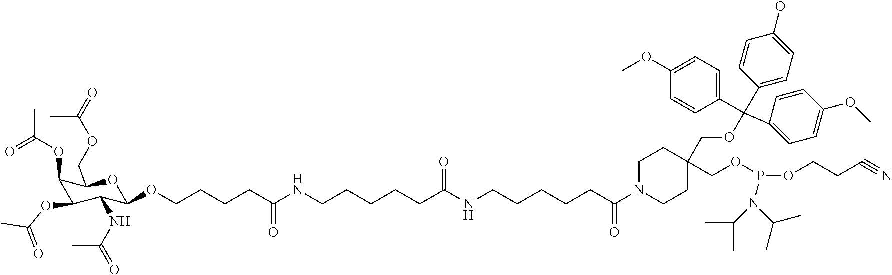

GalNAc-4 phosphoramidite

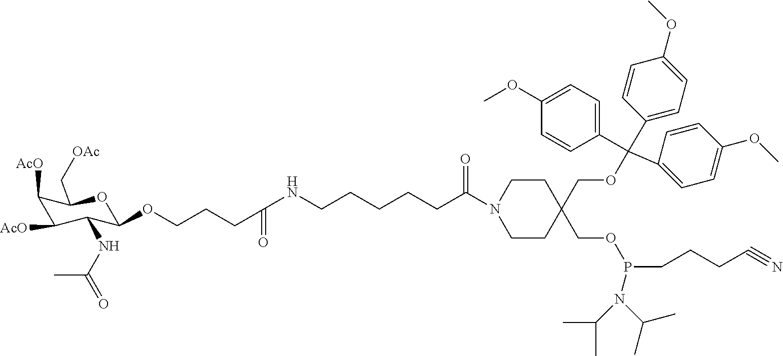

GalNAc-5 phosphoramidite

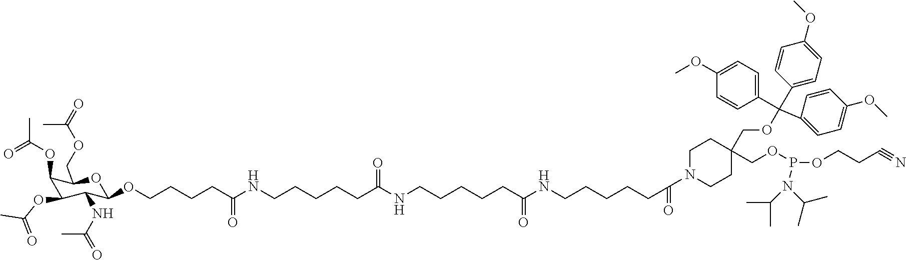

GalNAc-6 phosphoramidite
After Attachment to Oligos (Nomenclature)

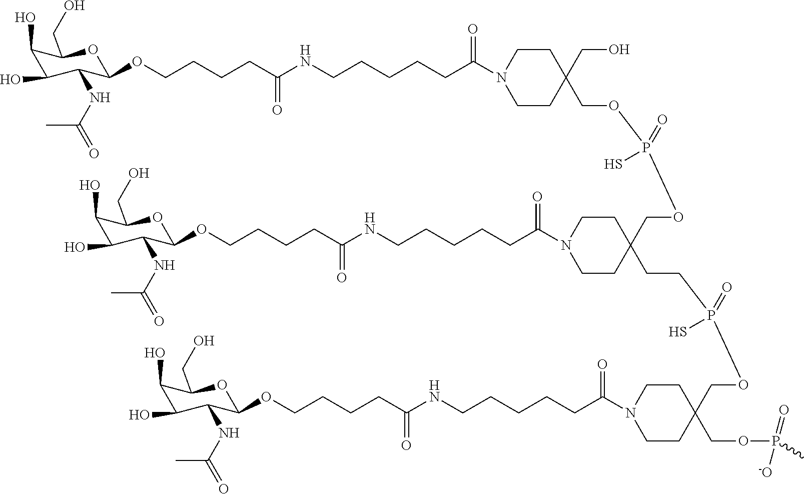

(GalNAc3-(PS)2-p)

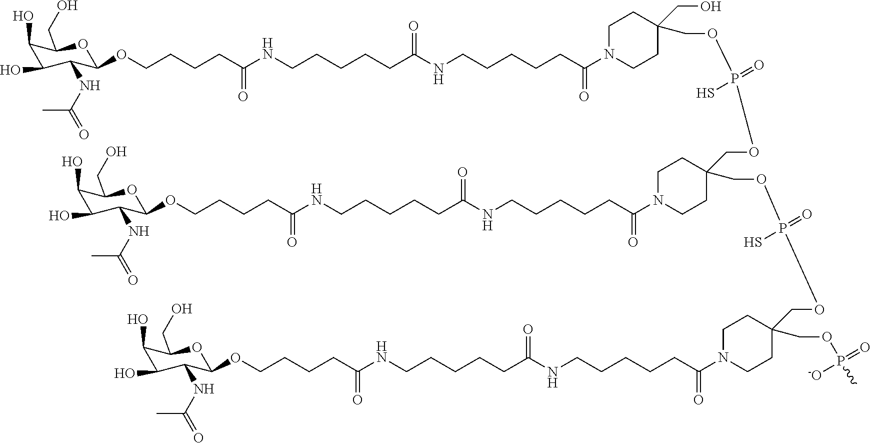

(GalNAc4-(PS)2-p)

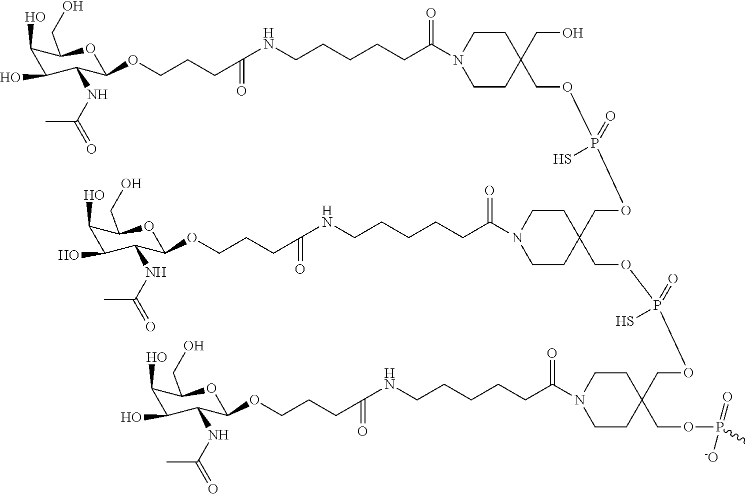

(GalNAc5-(PS)2-p)

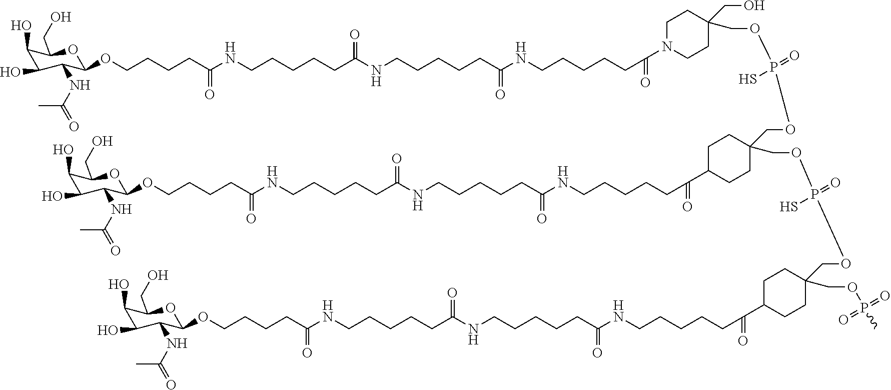

(GalNAc6-(PS)2-p)

In some embodiments, the galactosamine is attached to the 3′ end of the sense strand or first nucleotide sequence. In some embodiments, the galactosamine is attached to the 3′ end of the sense strand or first nucleotide sequence via 1, 2, 3, 4, or 5 or more linkers. In some embodiments, the galactosamine is attached to the 5′ end of the sense strand or first nucleotide sequence. In some embodiments, the galactosamine is attached to the 5′ end of the sense strand or first nucleotide sequence via 1, 2, 3, 4, or 5 or more linkers. In some embodiments, the galactosamine is attached to the 3′ end of the antisense strand or second nucleotide sequence. In some embodiments, the galactosamine is attached to the 3′ end of the antisense strand or second nucleotide sequence via 1, 2, 3, 4, or 5 or more linkers. In some embodiments, the galactosamine is attached to the 5′ end of the antisense strand or second nucleotide sequence. In some embodiments, the galactosamine is attached to the 5′ end of the antisense strand or second nucleotide sequence via 1, 2, 3, 4, or 5 or more linkers. In some embodiments, the one or more linkers are independently selected from the group consisting of a phosphodiester (p or po) linker, phosphorothioate (ps) linker, mesyl phosphoramidate linker (Ms), phosphoramidite (HEG) linker, triethylene glycol (TEG) linker, and/or phosphorodithioate linker. In some embodiments, the one or more linkers are independently selected from the group consisting of p-(PS)2, (PS)2-p-TEG-p, (PS)2-p-HEG-p, and (PS)2-p-(HEG-p)2.

In some embodiments, the conjugated moiety is a lipid moiety. In some embodiments, any of the siNAs disclosed herein are attached to a conjugated moiety that is a lipid moiety. Examples of lipid moieties include, but are not limited to, a cholesterol moiety, a thioether, e.g., hexyl-S-tritylthiol, a thiocholesterol, an aliphatic chain, e.g., dodecandiol or undecyl residues a phospholipid, e.g., di-hexadecyl-rac-glycerol or triethylammonium 1-di-O-hexadecyl-rac-glycero-S—H-phosphonate, a polyamine or a polyethylene glycol chain, adamantane acetic acid, a palmityl moiety, or an octadecylamine or hexylamino-carbonyl-oxycholesterol moiety.

In some embodiments, the conjugated moiety is an active drug substance. In some embodiments, any of the siNAs disclosed herein are attached to a conjugated moiety that is an active drug substance. Examples of active drug substances include, but are not limited to, aspirin, warfarin, phenylbutazone, ibuprofen, suprofen, fenbufen, ketoprofen, (5)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic.

### 5′-Stabilized End Cap

Further disclosed herein are siNA molecules comprising a 5′-stabilized end cap. As used herein the terms “5′-stabilized end cap” and “5′ end cap” are used interchangeably. In some embodiments, a 2′-O-methyl nucleotide in any of sense strands or first nucleotide sequences disclosed herein is replaced with a nucleotide containing a 5′-stabilized end cap. In some embodiments, a 2′-O-methyl nucleotide in any of antisense strands or second nucleotide sequences disclosed herein is replaced with a nucleotide containing a 5′-stabilized end cap. In some embodiments, a 2′-O-methyl nucleotide in any of sense strands or first nucleotide sequences disclosed herein is further modified to contain a 5′-stabilized end cap. In some embodiments, a 2′-O-methyl nucleotide in any of antisense strands or second nucleotide sequences disclosed herein is further modified to contain a 5′-stabilized end cap.

In some embodiments, the 5′-stabilized end cap is a 5′ phosphate mimic. In some embodiments, the 5′-stabilized end cap is a modified 5′ phosphate mimic. In some embodiments, the modified 5′ phosphate is a chemically modified 5′ phosphate. In some embodiments, the 5′-stabilized end cap is a 5′-vinyl phosphonate. In some embodiments, the 5′-vinyl phosphonate is a 5′-(E)-vinyl phosphonate or 5′-(Z)-vinyl phosphonate. In some embodiments, the 5′-vinyl phosphonate is a deuterated vinyl phosphonate. In some embodiments, the deuterated vinyl phosphonate is a mono-deuterated vinyl phosphonate. In some embodiments, the deuterated vinyl phosphonate is a di-deuterated vinyl phosphonate. In some embodiments, the 5′-stabilized end cap is a phosphate mimic. Examples of phosphate mimics are disclosed in Parmar et al., J Med Chem, 201861(3):734-744, International Publication Nos. WO2018/045317 and WO2018/044350, and U.S. Pat. No. 10,087,210, each of which is incorporated by reference in its entirety.

In some aspects, the present disclosure provides siNA comprising a nucleotide phosphate mimic selected from:

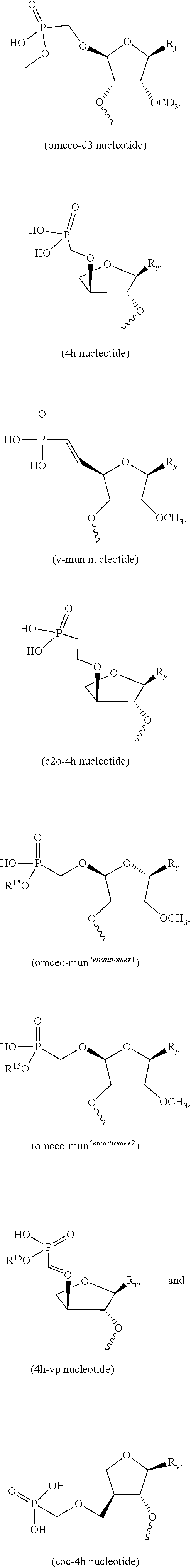

wherein R_{y} is a nucleobase and R¹⁵ is H or CH₃. In some embodiments, the nucleobase is selected from thymine, cytosine, guanine, adenine, uracil, and an analogue or derivative thereof. In some embodiments, the disclosed nucleotide phosphate mimics include, but are not limited to, the structures:

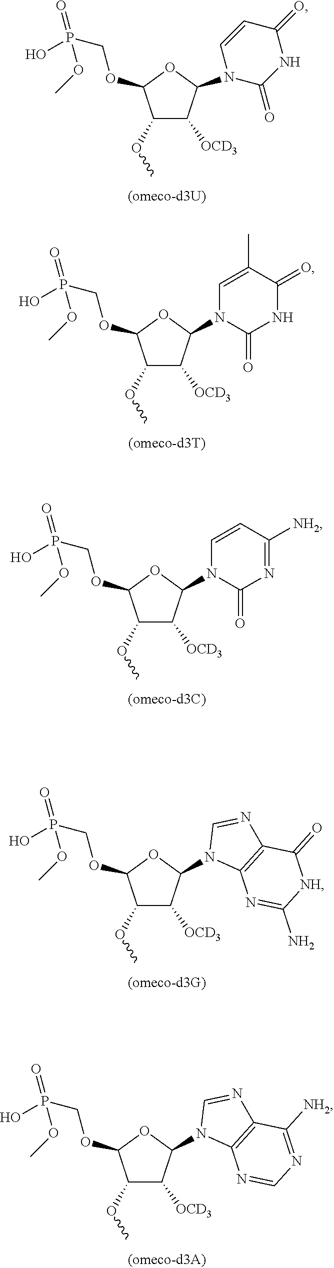

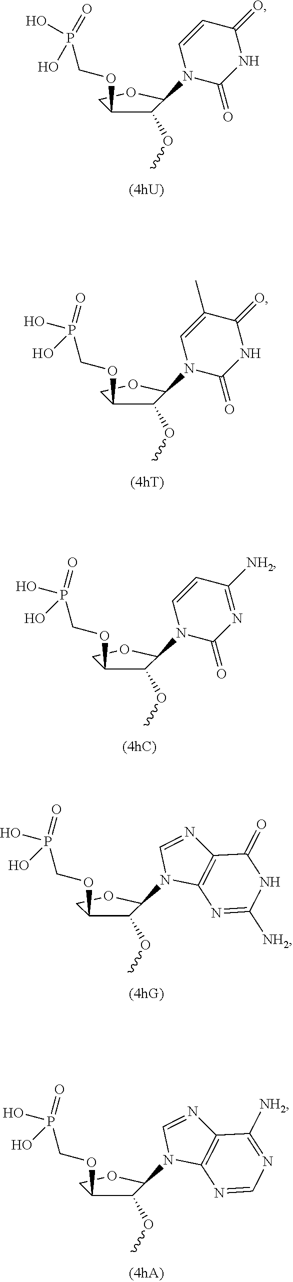

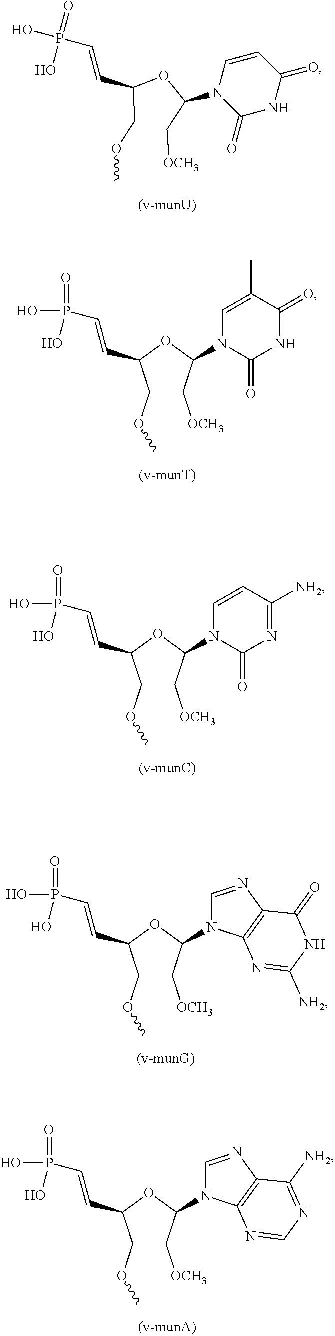

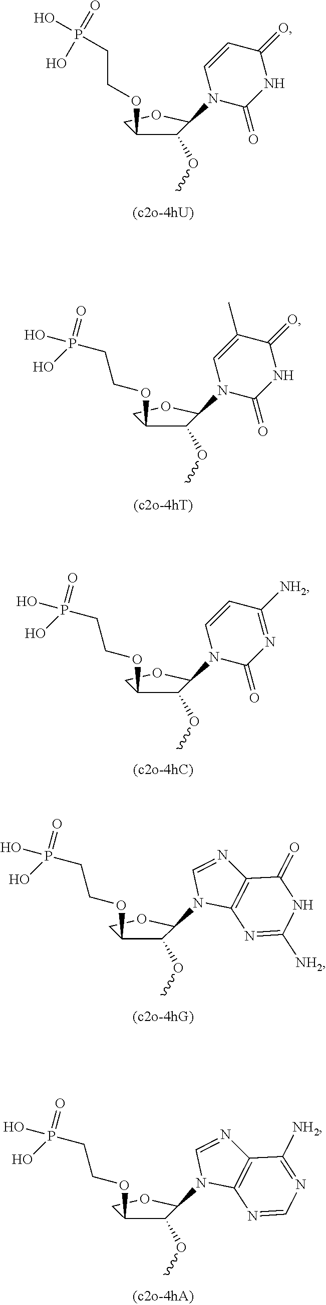

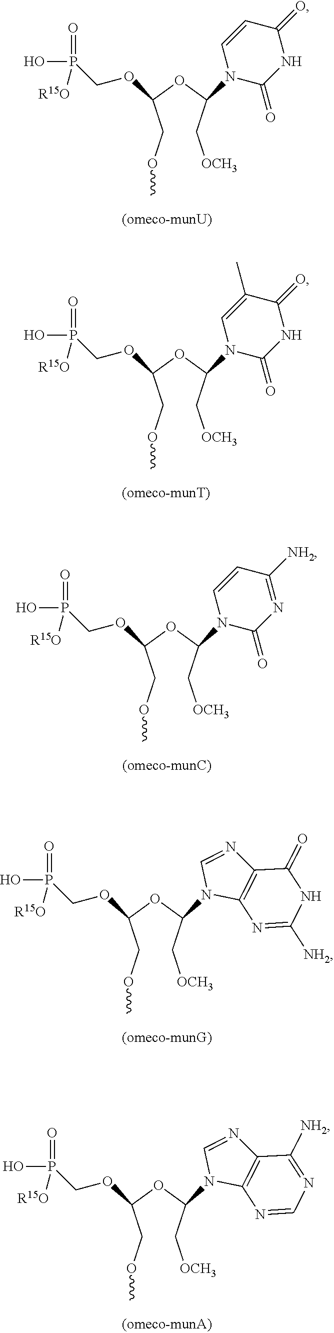

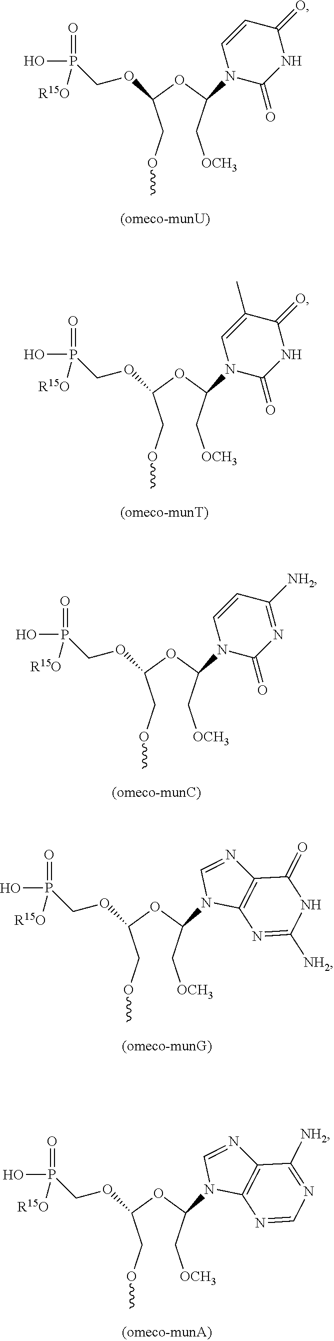

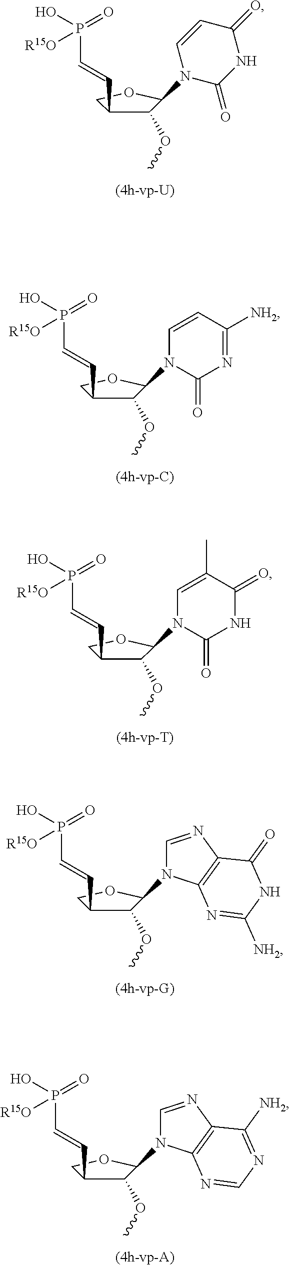

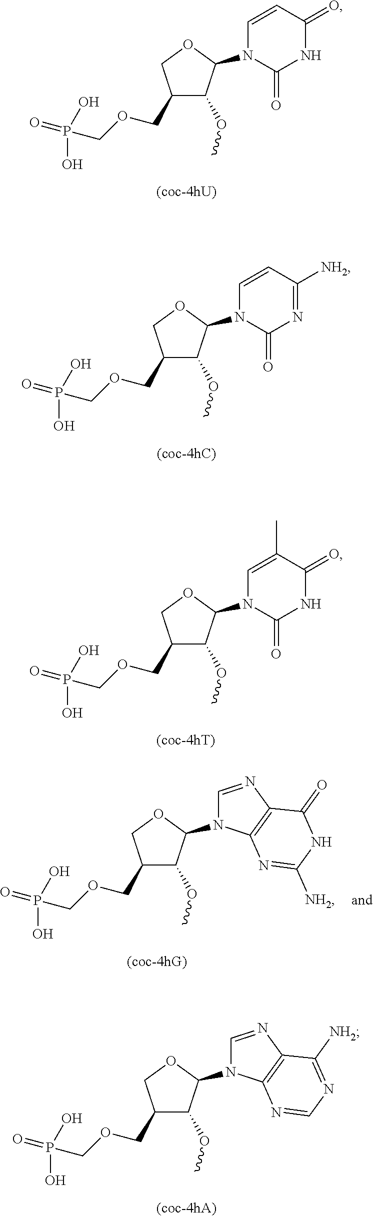

wherein R¹⁵ is H or CH₃.
In some aspects, the present disclosure provides siNA comprising a nucleotide phosphate mimic selected from:

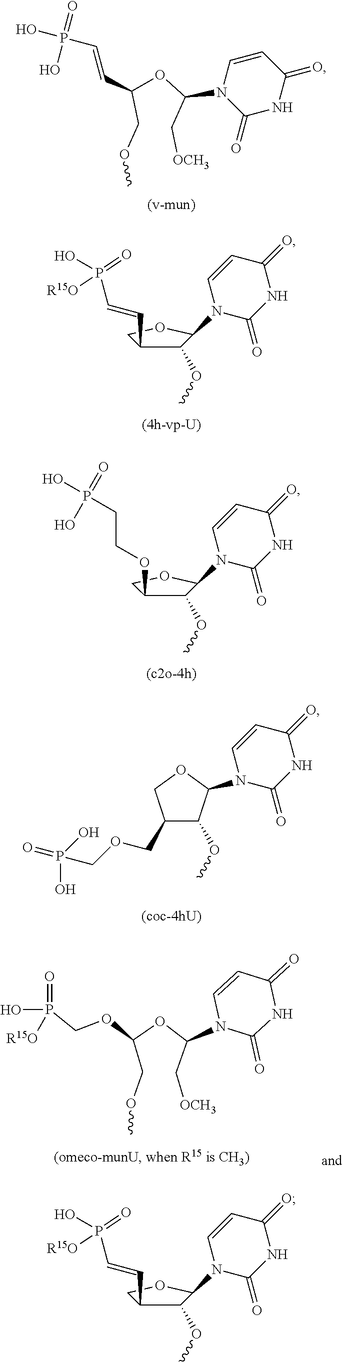

where R¹⁵ is H or CH₃. In some embodiments, one of these novel nucleotide phosphate mimics (e.g., omeco-d3 nucleotide, 4h nucleotide, v-mun nucleotide, c2o-4h nucleotide, coc-4h nucleotide, omeco-mun nucleotide, 4h-vp nucleotide, or d2vm nucleotide) are located at the 5′ end of the antisense strand; however, these novel nucleotide phosphate mimics may also be incorporated at the 5′ end of the sense strand, the 3′ end of the antisense strand, or the 3′ end of the sense strand.

Additionally or alternatively, the siNA molecules disclosed herein may comprise in the sense strand, the antisense strand, or both a 5′-stabilized end cap of Formula (Ia):

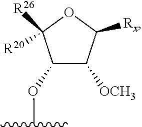

wherein Rₓ is H, a nucleobase, aryl, or heteroaryl; R²⁶ is

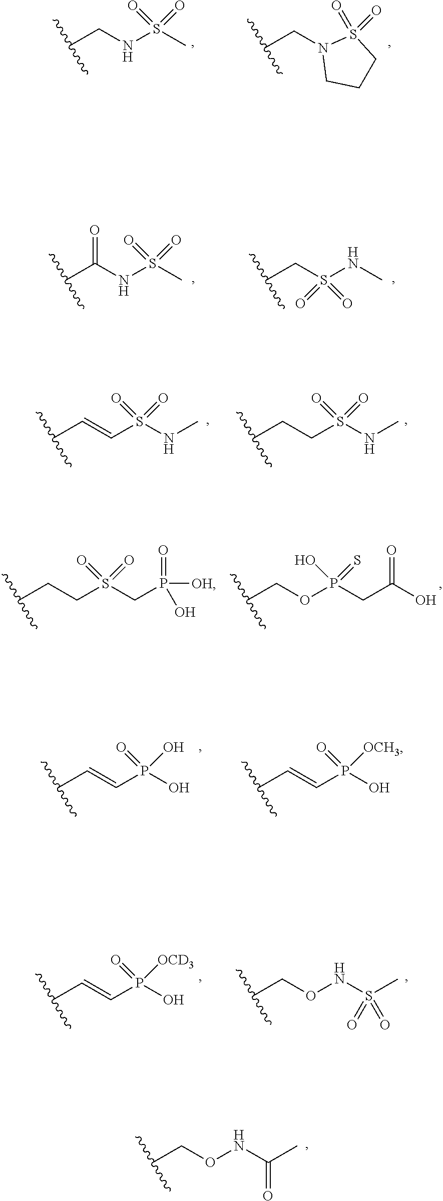

—CH═CD-Z, —CD═CH—Z, —CD═CD-Z, —(CR²¹R²²)ₙ—Z, or —(C₂-C₆ alkenylene)-Z and R²⁰ is H; or R²⁶ and R²⁰ together form a 3- to 7-membered carbocyclic ring substituted with —(CR²¹R²²)ₙ—Z or —(C₂-C₆ alkenylene)-Z; n is 1, 2, 3, or 4; Z is —ONR²³R²⁴, —OP(O)OH(CH₂)ₘCO₂R²³, —OP(S)OH(CH₂)ₘCO₂R²³, —P(O)(OH)2, —P(O)(OH)(OCH₃), —P(O)(OH)(OCD₃), —SO₂(CH₂)ₘP(O)(OH)₂, —SO₂NR²³R²⁵, —NR²³R²⁴, —NR²³SO₂R²⁴; either R²¹ and R²² are independently hydrogen or C₁-C₆ alkyl, or R²¹ and R²² together form an oxo group; R²³ is hydrogen or C₁-C₆ alkyl; R²⁴ is —SO₂R²⁵ or —C(O)R²⁵; or R²³ and R²⁴ together with the nitrogen to which they are attached form a substituted or unsubstituted heterocyclic ring; R²⁵ is C₁-C₆ alkyl; and m is 1, 2, 3, or 4. In some embodiments, R¹ is an aryl. In some embodiments, the aryl is a phenyl.

Additionally or alternatively, the siNA molecules disclosed herein may comprise in the sense strand, the antisense strand, or both a 5′-stabilized end cap of Formula (Ib):

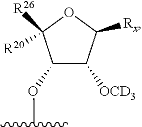

wherein Rₓ is H, a nucleobase, aryl, or heteroaryl; R²⁶ is

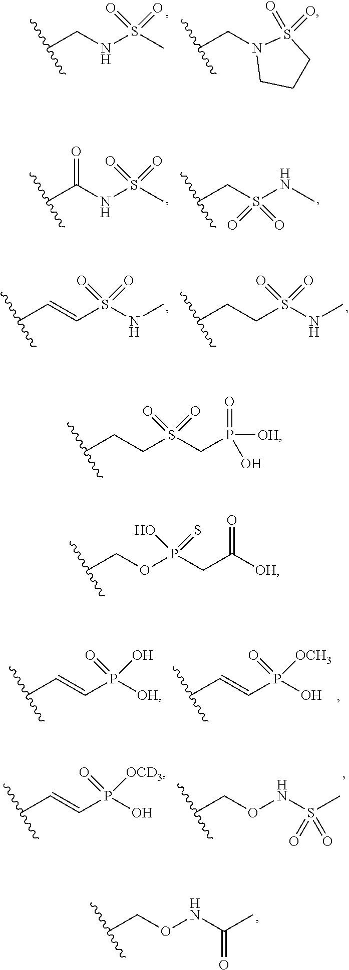

—CH═CD-Z, —CD═CH—Z, —CD═CD-Z, —(CR²¹R²²)ₙ—Z, or —(C₂-C₆ alkenylene)-Z and R²⁰ is H; or R²⁶ and R²⁰ together form a 3- to 7-membered carbocyclic ring substituted with —(CR²¹R²²)ₙ—Z or —(C₂-C₆ alkenylene)-Z; n is 1, 2, 3, or 4; Z is —ONR²³R²⁴, —OP(O)OH(CH₂)ₘCO₂R²³, —OP(S)OH(CH₂)ₘCO₂R²³, —P(O)(OH)₂, —P(O)(OH)(OCH₃), —P(O)(OH)(OCD₃), —SO₂(CH₂)ₘP(O)(OH)2, —SO₂NR²³R²⁵, —NR²³R²⁴, —NR²³SO₂R²⁴; either R²¹ and R²² are independently hydrogen or C₁-C₆ alkyl, or R²¹ and R²² together form an oxo group; R²³ is hydrogen or C₁-C₆ alkyl; R²⁴ is —SO₂R²⁵ or —C(O)R²⁵; or R²³ and R²⁴ together with the nitrogen to which they are attached form a substituted or unsubstituted heterocyclic ring; R²⁵ is C₁-C₆ alkyl; and m is 1, 2, 3, or 4. In some embodiments, R¹ is an aryl. In some embodiments, the aryl is a phenyl.

Additionally or alternatively, the siNA molecules disclosed herein may comprise in the sense strand, the antisense strand, or both a 5′-stabilized end cap of Formula (Ic):

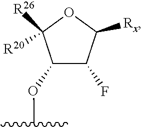

wherein Rₓ is a nucleobase, aryl, heteroaryl, or H, R²⁶ is

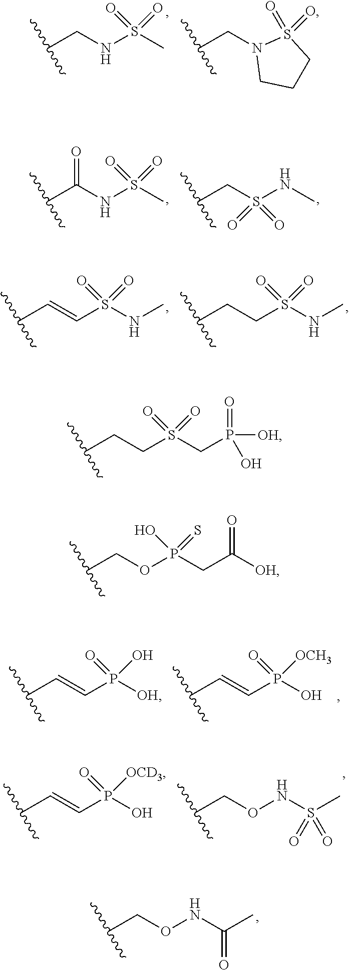

—CH═CD-Z, —CD═CH—Z, —CD═CD-Z, —(CR²¹R²²)ₙ—Z, or —(C₂-C₆alkenylene)-Z and R²⁰ is hydrogen; or R²⁶ and R²⁰ together form a 3- to 7-membered carbocyclic ring substituted with —(CR²¹R²²)ₙ—Z or —(C₂-C₆ alkenylene)-Z; n is 1, 2, 3, or 4; Z is —ONR²³R²⁴, —OP(O)OH(CH₂)ₘCO₂R²³, —OP(S)OH(CH₂)ₘCO₂R²³, —P(O)(OH)₂, —P(O)(OH)(OCH₃), —P(O)(OH)(OCD₃), —SO₂(CH₂)ₘP(O)(OH)₂, —SO₂NR²³R²⁵, —NR²³R²⁴, or —NR²³SO₂R²⁴; R²¹ and R²² either are independently hydrogen or C₁-C₆ alkyl, or R²¹ and R²² together form an oxo group; R²³ is hydrogen or C₁-C₆ alkyl; R²⁴ is —SO₂R²⁵ or —C(O)R²⁵; or
R²³ and R²⁴ together with the nitrogen to which they are attached form a substituted or unsubstituted heterocyclic ring; R²⁵ is C₁-C₆ alkyl; and m is 1, 2, 3, or 4. In some embodiments, R¹ is an aryl. In some embodiments, the aryl is a phenyl.

Additionally or alternatively, the siNA molecules disclosed herein may comprise in the sense strand, the antisense strand, or both a 5′-stabilized end cap of Formula (IIa):

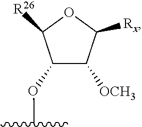

wherein Rₓ is a nucleobase, aryl, heteroaryl, or H, R²⁶ is

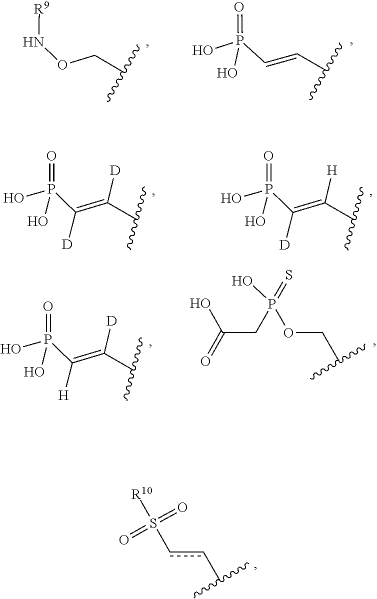

—CH₂SO₂NHCH₃, or

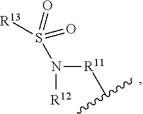

R⁹ is —SO₂CH₃ or —COCH₃, [symbol] is a double or single bond, R¹⁰=—CH₂PO₃H or —NHCH₃, R¹¹ is —CH₂— or —CO—, and R¹² is H and R¹³ is CH₃ or R¹² and R¹³ together form —CH₂CH₂CH₂—. In some embodiments, R¹ is an aryl. In some embodiments, the aryl is a phenyl.

Additionally or alternatively, the siNA molecules disclosed herein may comprise in the sense strand, the antisense strand, or both a 5′-stabilized end cap of Formula (IIb):

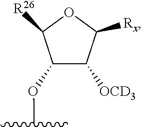

wherein Rₓ is a nucleobase, aryl, heteroaryl, or H, R²⁶ is

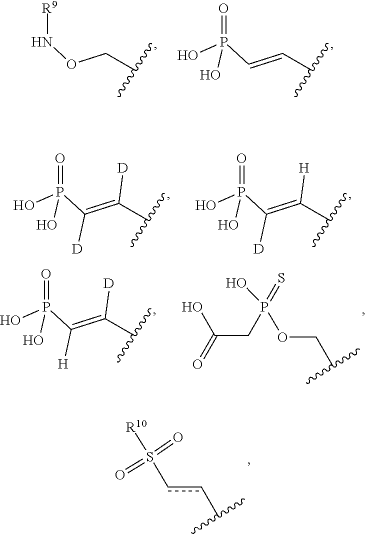

—CH₂SO₂NHCH₃, or

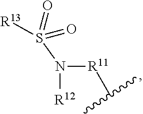

R⁹ is —SO₂CH₃ or —COCH₃, [symbol] is a double or single bond, R¹⁰=—CH₂PO₃H or —NHCH₃, R¹¹ is —CH₂— or —CO—, and R¹² is H and R¹³ is CH₃ or R¹² and R¹³ together form —CH₂CH₂CH₂—. In some embodiments, R¹ is an aryl. In some embodiments, the aryl is a phenyl.

Additionally or alternatively, the siNA molecules disclosed herein may comprise in the sense strand, the antisense strand, or both a 5′-stabilized end cap of Formula (III):

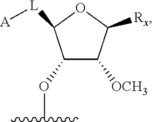

wherein Rₓ is a nucleobase, aryl, heteroaryl, or H, L is —CH₂—, —CH═CH—, —CO—, or —CH₂CH₂—, and A is —ONHCOCH₃, —ONHSO₂CH₃, —PO₃H, —OP(SOH)CH₂CO₂H, —SO₂CH₂PO₃H, —SO₂NHCH₃, —NHSO₂CH₃, or —N(SO₂CH₂CH₂CH₂). In some embodiments, R¹ is an aryl. In some embodiments, the aryl is a phenyl.

Additionally or alternatively, the siNA molecules disclosed herein may comprise a 5′-stabilized end cap selected from the group consisting of Formula (1) to Formula (16), Formula (9X) to Formula (12X), Formula (16X), Formula (9Y) to Formula (12Y), Formula (16Y), Formula (21) to Formula (36), Formula 36X, Formula (41) to (56), Formula (49X) to (52X), Formula (49Y) to (52Y), Formula 56X, Formula 56Y, Formula (61) and Formula (62):

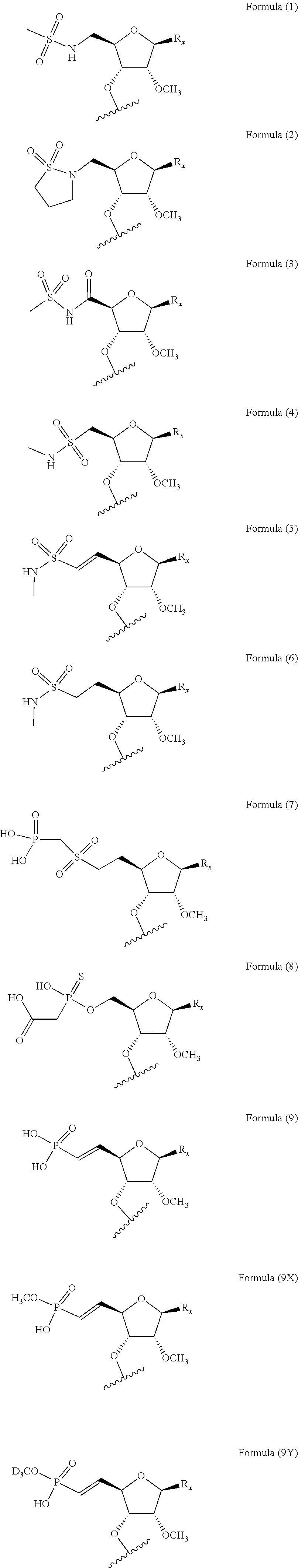

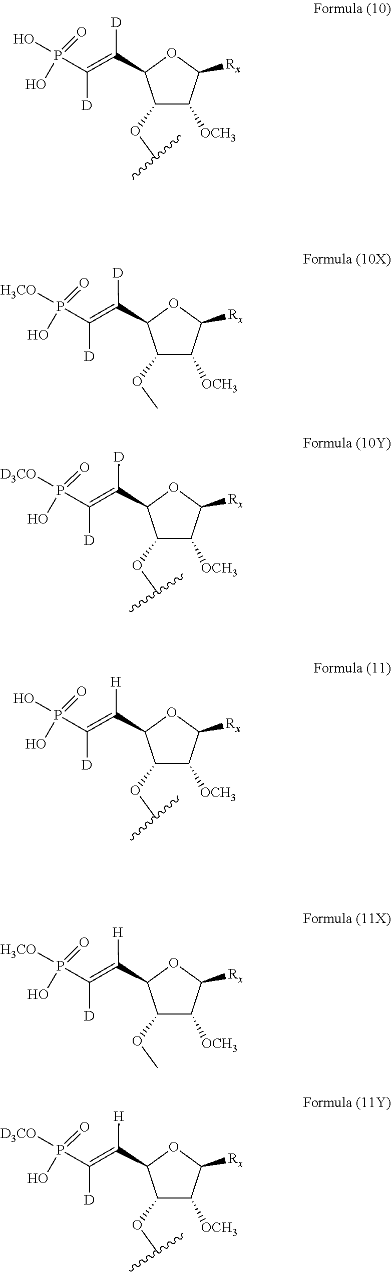

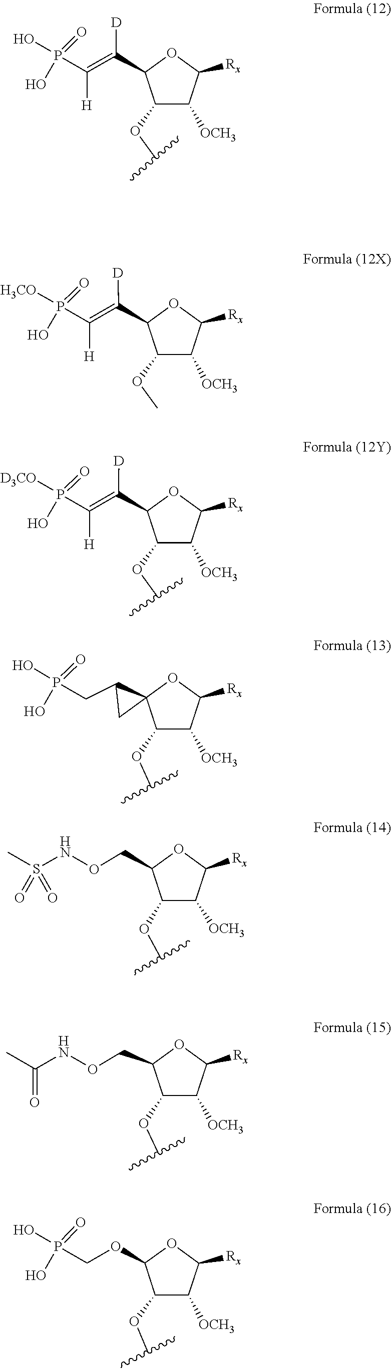

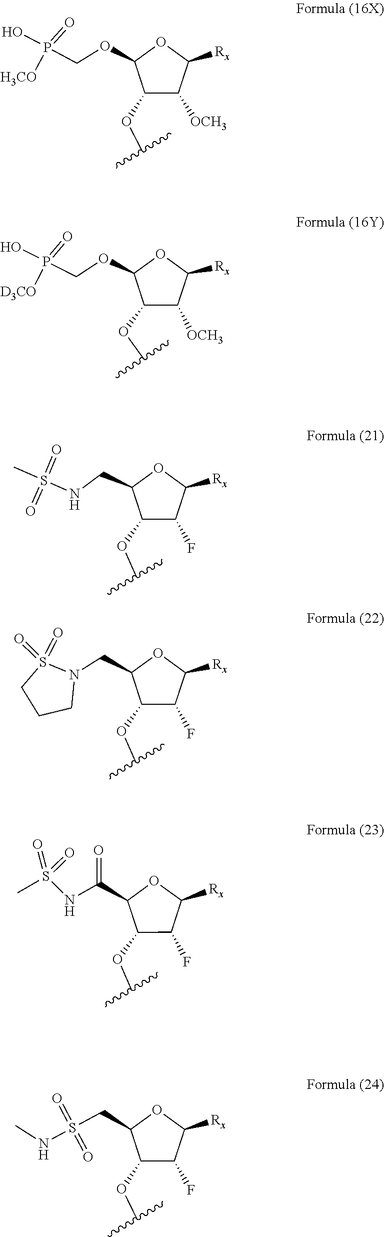

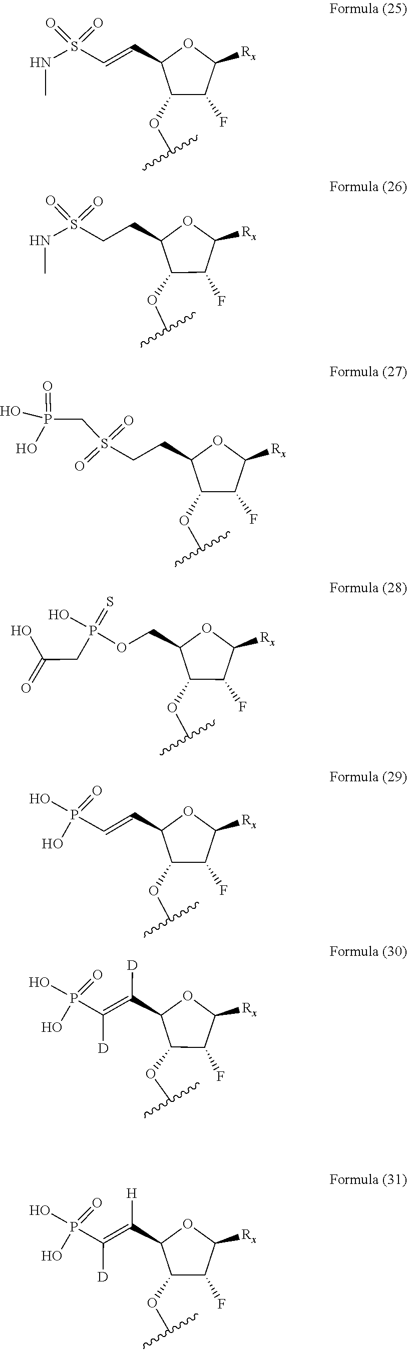

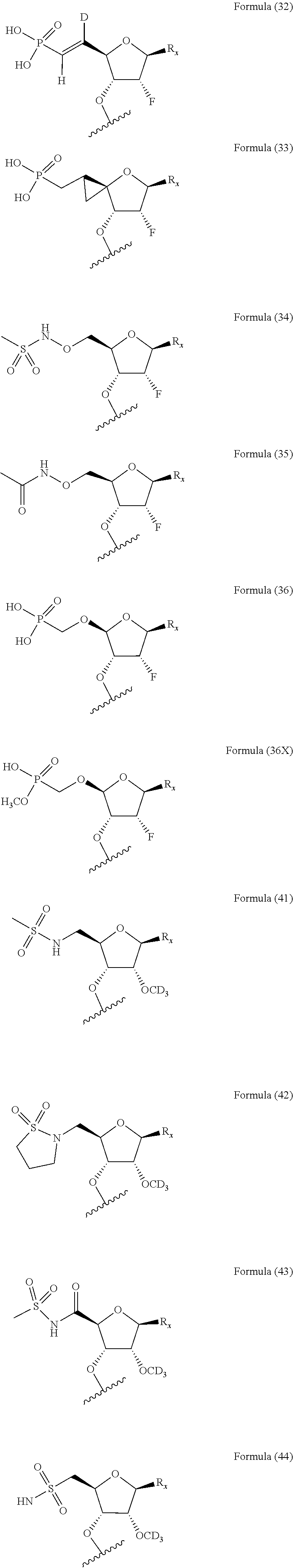

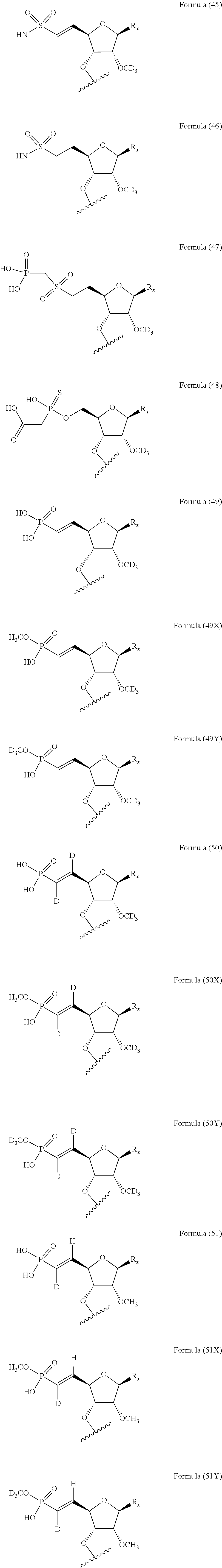

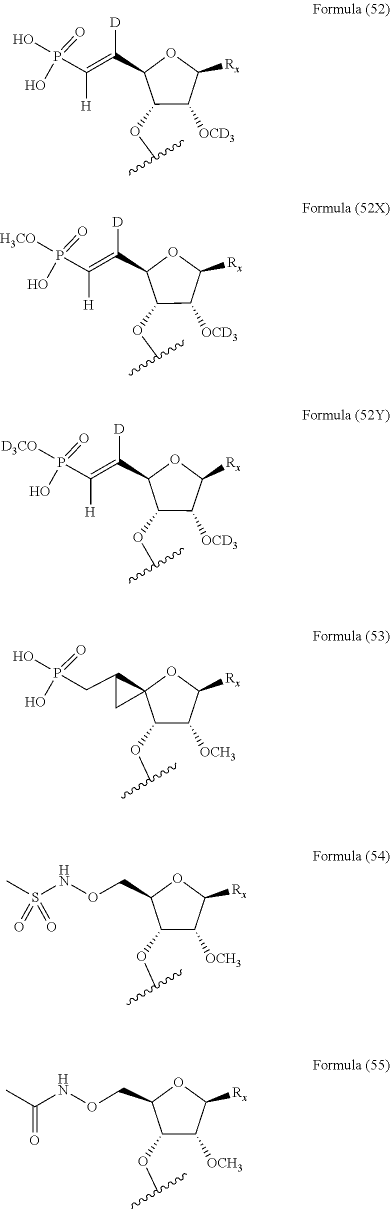

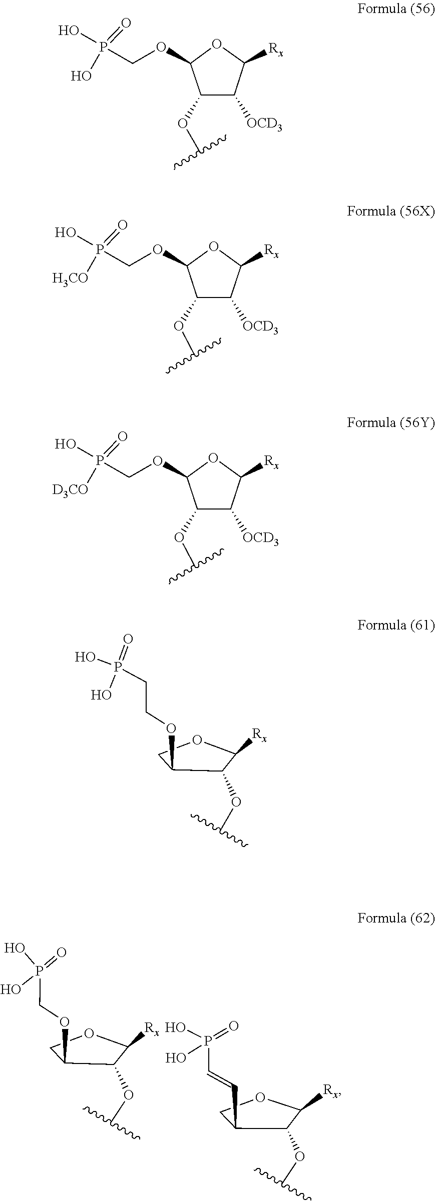

wherein Rₓ is a nucleobase, aryl, heteroaryl, or H.

In some embodiments, any of the siNA molecules disclosed herein comprise a 5′-stabilized end cap selected from the group consisting of Formula (50), Formula (50X), Formula (50Y), Formula (56), Formula (56X), Formula (56Y), Formula (61), Formula (62), and Formula (63):

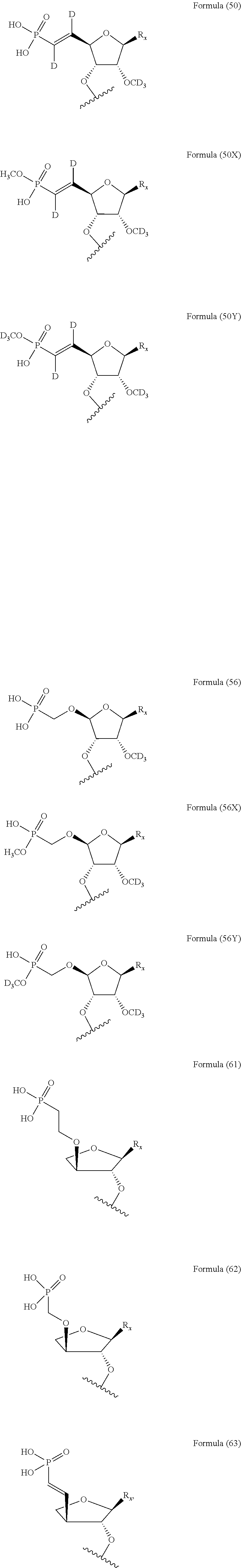

wherein Rₓ, is a nucleobase, aryl, heteroaryl, or H.

In some embodiments, any of the siNA molecules disclosed herein comprise a 5′-stabilized end cap selected from the group consisting of Formula (71) to Formula (86), Formula (79X) to Formula (82X), Formula (79Y) to (82Y), Formula 86X, Formula 86X′, Formula 86Y, and Formula 86Y′:

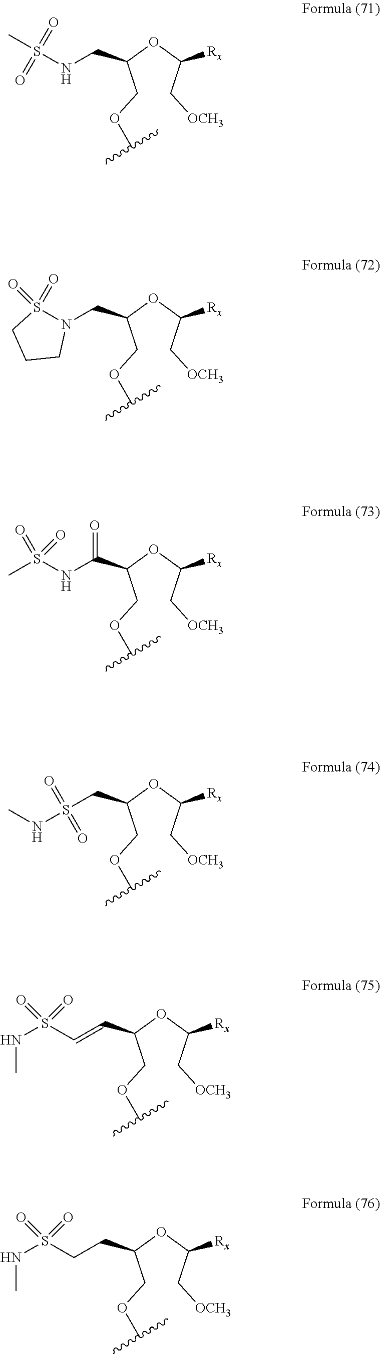

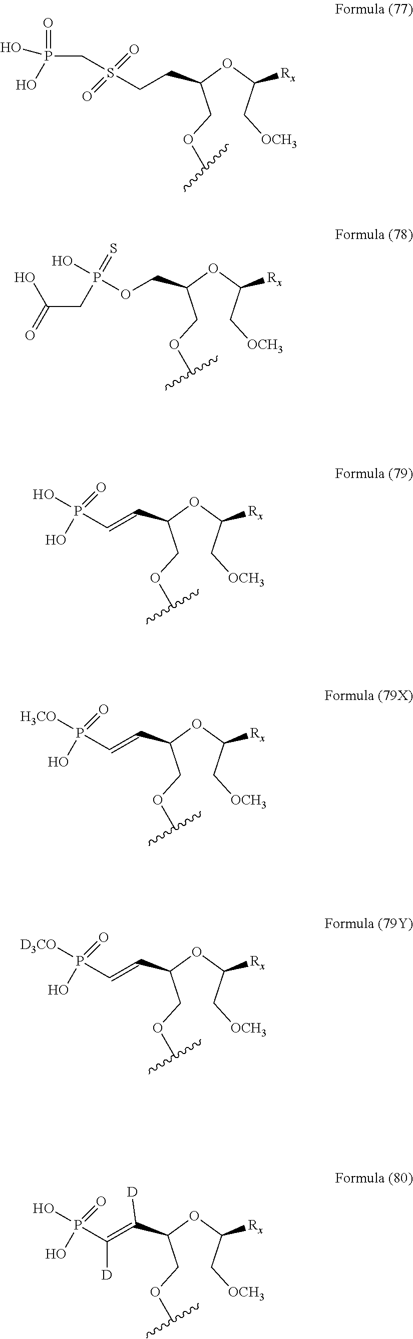

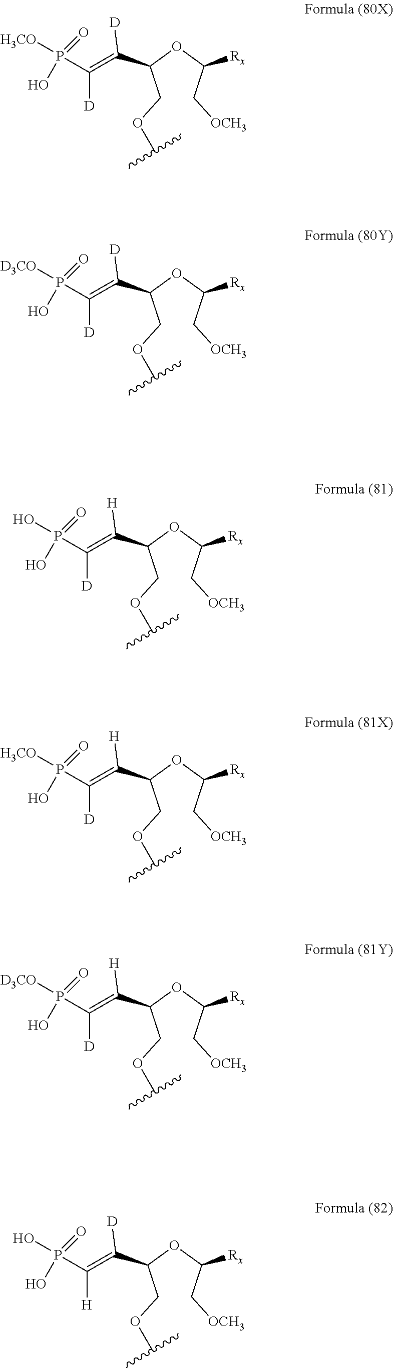

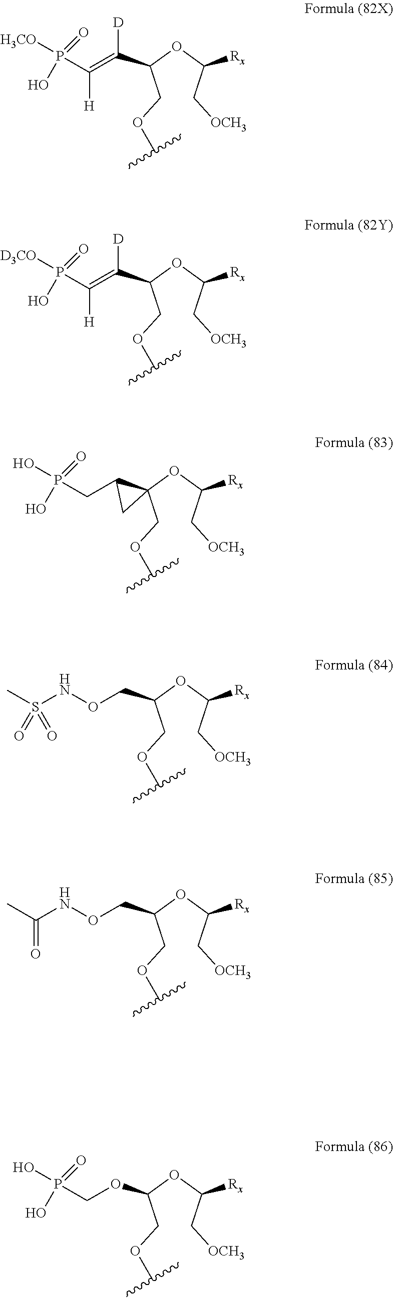

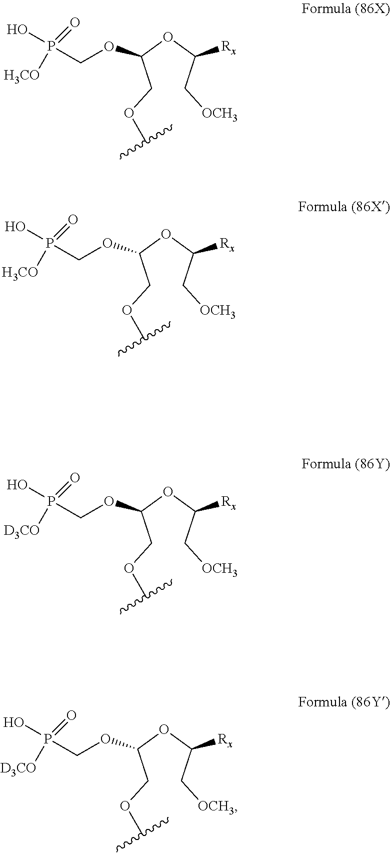

wherein Rₓ is a nucleobase, aryl, heteroaryl, or H.

In some embodiments, any of the siNA molecules disclosed herein comprise a 5′-stabilized end cap selected from the group consisting of Formula (78), Formula (79), Formula (79X), Formula (79Y), Formula (86), Formula (86X), and Formula (86X′):

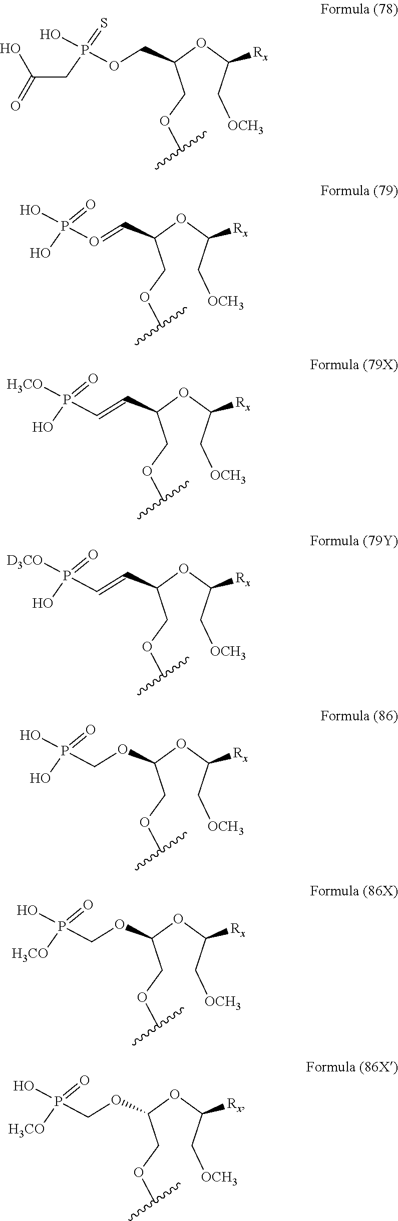

wherein Rₓ is a nucleobase, aryl, heteroaryl, or H.

In some embodiments, any of the siNA molecules disclosed herein comprise a 5′-stabilized end cap selected from the group consisting of Formulas (1A)-(15A), Formulas (1A-1)-(7A-1), Formulas (1A-2)-(7A-2), Formulas (1A-3)-(7A-3), Formulas (1A-4)-(7A-4), Formulas (9B)-(12B), Formulas (9AX)-(12AX), Formulas (9AY)-(12AY), Formulas (9BX)-(12BX), and Formulas (9BY)-(12BY):

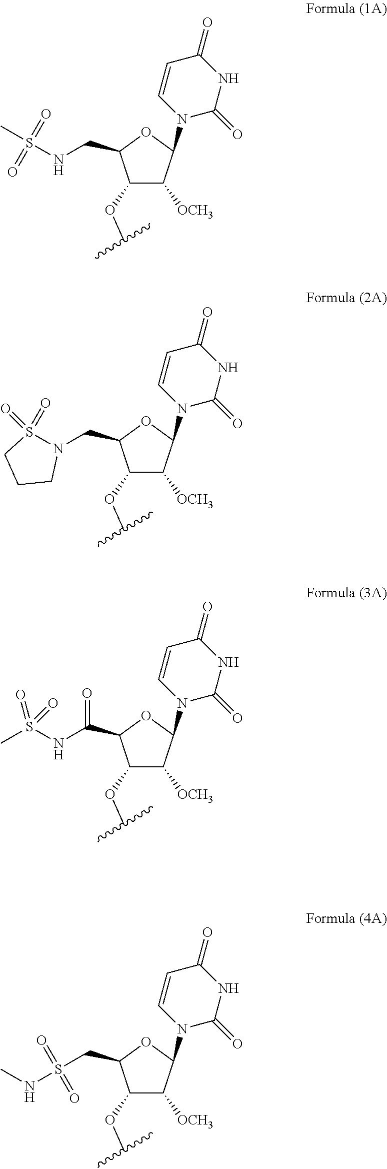 [SMILES: *O[C@H]1[C@@H](OC)[C@H](n2ccc(=O)[nH]c2=O)O[C@@H]1CNS(C)(=O)=O ; *O[C@H]1[C@@H](OC)[C@H](n2ccc(=O)[nH]c2=O)O[C@@H]1CN1CCCS1(=O)=O ; *O[C@H]1[C@@H](OC)[C@H](n2ccc(=O)[nH]c2=O)O[C@@H]1C(=O)NS(C)(=O)=O ; *O[C@H]1[C@@H](OC)[C@H](n2ccc(=O)[nH]c2=O)O[C@@H]1CS(=O)(=O)NC]

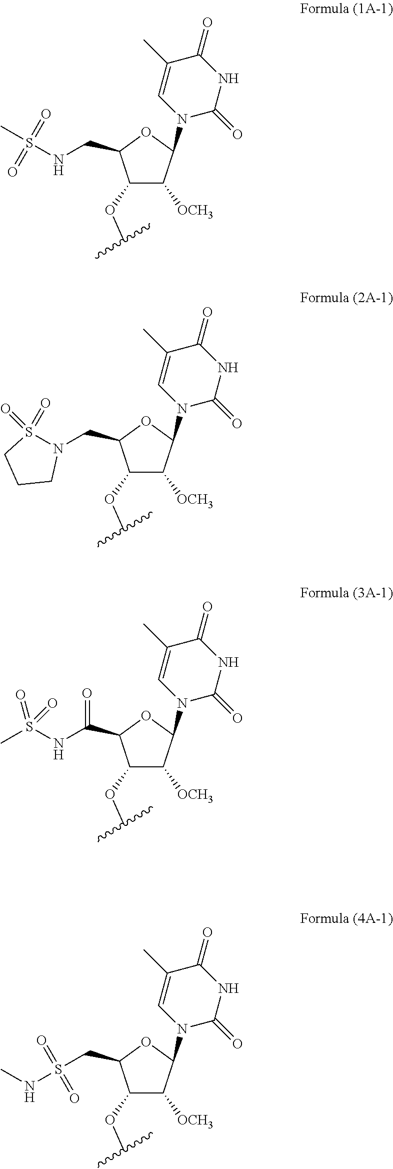 [SMILES: *O[C@H]1[C@@H](OC)[C@H](n2cc(C)c(=O)[nH]c2=O)O[C@@H]1CNS(C)(=O)=O ; *O[C@H]1[C@@H](OC)[C@H](n2cc(C)c(=O)[nH]c2=O)O[C@@H]1CN1CCCS1(=O)=O ; *O[C@H]1[C@@H](OC)[C@H](n2cc(C)c(=O)[nH]c2=O)O[C@@H]1C(=O)NS(C)(=O)=O ; *O[C@H]1[C@@H](OC)[C@H](n2cc(C)c(=O)[nH]c2=O)O[C@@H]1CS(=O)(=O)NC]

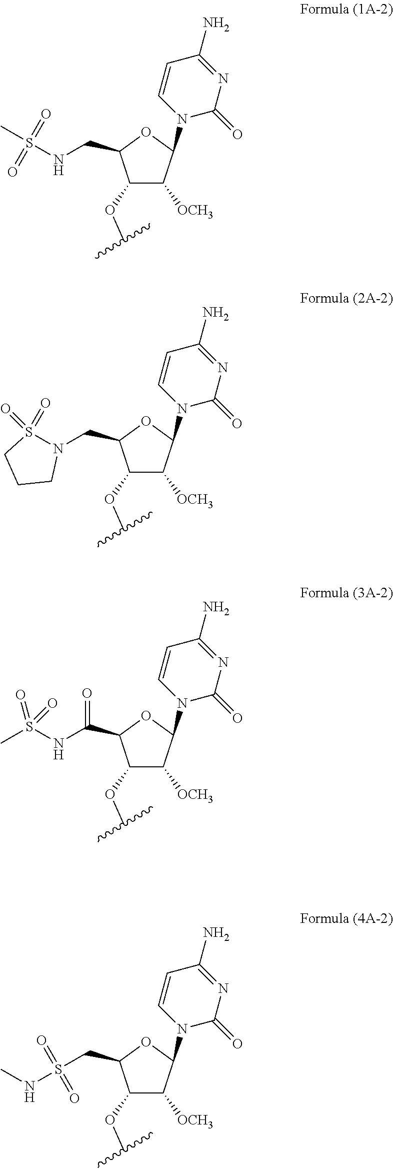 [SMILES: *O[C@H]1[C@@H](OC)[C@H](n2ccc(N)nc2=O)O[C@@H]1CNS(C)(=O)=O ; *O[C@H]1[C@@H](OC)[C@H](n2ccc(N)nc2=O)O[C@@H]1CN1CCCS1(=O)=O ; *O[C@H]1[C@@H](OC)[C@H](n2ccc(N)nc2=O)O[C@@H]1C(=O)NS(C)(=O)=O ; *O[C@H]1[C@@H](OC)[C@H](n2ccc(N)nc2=O)O[C@@H]1CS(=O)(=O)NC]

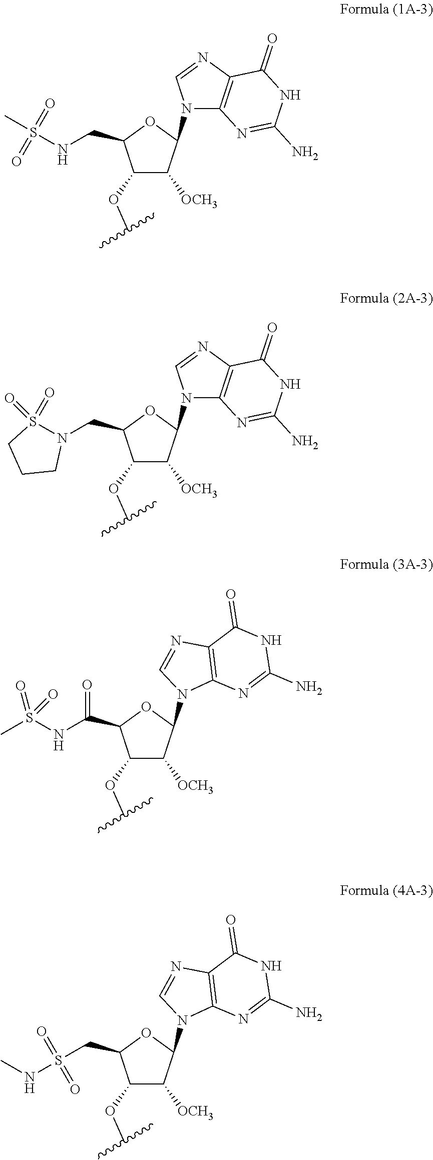 [SMILES: *O[C@H]1[C@@H](OC)[C@H](n2cnc3c(=O)[nH]c(N)nc32)O[C@@H]1CNS(C)(=O)=O ; *O[C@H]1[C@@H](OC)[C@H](n2cnc3c(=O)[nH]c(N)nc32)O[C@@H]1CN1CCCS1(=O)=O ; *O[C@H]1[C@@H](OC)[C@H](n2cnc3c(=O)[nH]c(N)nc32)O[C@@H]1C(=O)NS(C)(=O)=O ; *O[C@H]1[C@@H](OC)[C@H](n2cnc3c(=O)[nH]c(N)nc32)O[C@@H]1CS(=O)(=O)NC]

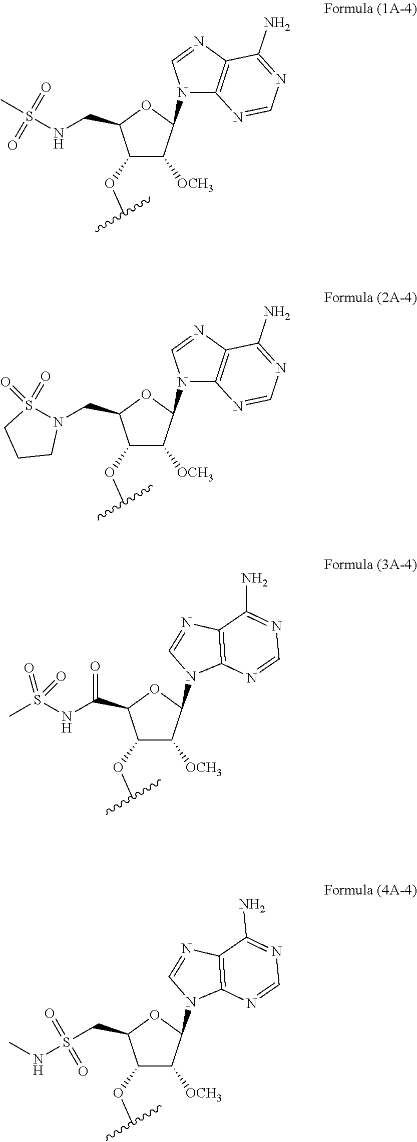 [SMILES: *O[C@H]1[C@@H](OC)[C@H](n2cnc3c(N)ncnc32)O[C@@H]1CNS(C)(=O)=O ; *O[C@H]1[C@@H](OC)[C@H](n2cnc3c(N)ncnc32)O[C@@H]1CN1CCCS1(=O)=O ; *O[C@H]1[C@@H](OC)[C@H](n2cnc3c(N)ncnc32)O[C@@H]1C(=O)NS(C)(=O)=O ; *O[C@H]1[C@@H](OC)[C@H](n2cnc3c(N)ncnc32)O[C@@H]1CS(=O)(=O)NC]

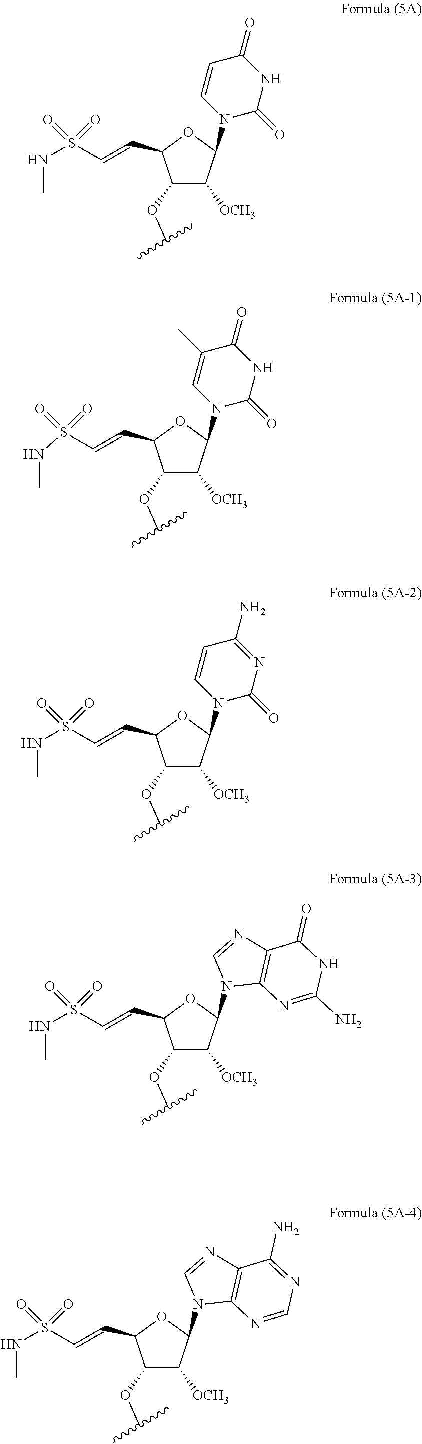 [SMILES: *O[C@H]1[C@@H](OC)[C@H](n2ccc(=O)[nH]c2=O)O[C@@H]1/C=C/S(=O)(=O)NC ; *O[C@H]1[C@@H](OC)[C@H](n2cc(C)c(=O)[nH]c2=O)O[C@@H]1/C=C/S(=O)(=O)NC ; *O[C@H]1[C@@H](OC)[C@H](n2ccc(N)nc2=O)O[C@@H]1/C=C/S(=O)(=O)NC ; *O[C@H]1[C@@H](OC)[C@H](n2cnc3c(=O)[nH]c(N)nc32)O[C@@H]1/C=C/S(=O)(=O)NC ; *O[C@H]1[C@@H](OC)[C@H](n2cnc3c(N)ncnc32)O[C@@H]1/C=C/S(=O)(=O)NC]

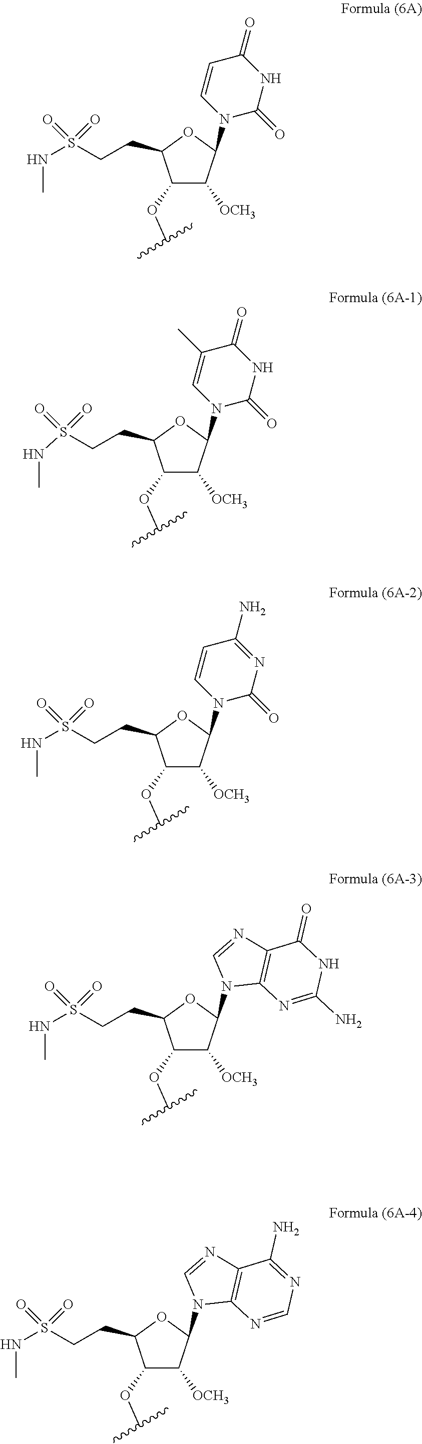 [SMILES: *O[C@H]1[C@@H](OC)[C@H](n2ccc(=O)[nH]c2=O)O[C@@H]1CCS(=O)(=O)NC ; *O[C@H]1[C@@H](OC)[C@H](n2cc(C)c(=O)[nH]c2=O)O[C@@H]1CCS(=O)(=O)NC ; *O[C@H]1[C@@H](OC)[C@H](n2ccc(N)nc2=O)O[C@@H]1CCS(=O)(=O)NC ; *O[C@H]1[C@@H](OC)[C@H](n2cnc3c(=O)[nH]c(N)nc32)O[C@@H]1CCS(=O)(=O)NC ; *O[C@H]1[C@@H](OC)[C@H](n2cnc3c(N)ncnc32)O[C@@H]1CCS(=O)(=O)NC]

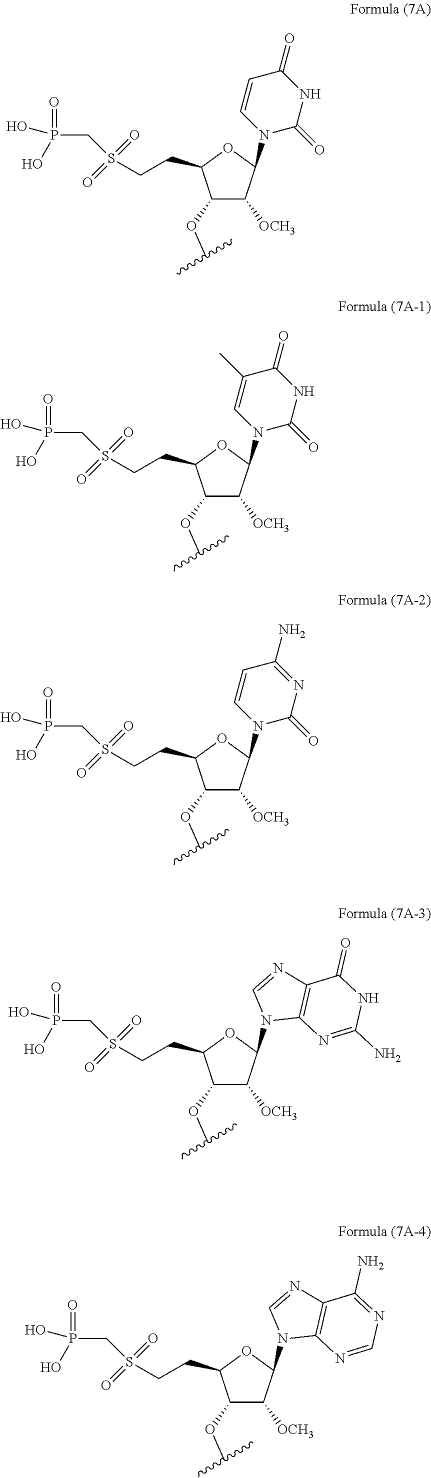 [SMILES: *O[C@H]1[C@@H](OC)[C@H](n2ccc(=O)[nH]c2=O)O[C@@H]1CCS(=O)(=O)CP(=O)(O)O ; *O[C@H]1[C@@H](OC)[C@H](n2cc(C)c(=O)[nH]c2=O)O[C@@H]1CCS(=O)(=O)CP(=O)(O)O ; *O[C@H]1[C@@H](OC)[C@H](n2ccc(N)nc2=O)O[C@@H]1CCS(=O)(=O)CP(=O)(O)O ; *O[C@H]1[C@@H](OC)[C@H](n2cnc3c(=O)[nH]c(N)nc32)O[C@@H]1CCS(=O)(=O)CP(=O)(O)O ; *O[C@H]1[C@@H](OC)[C@H](n2cnc3c(N)ncnc32)O[C@@H]1CCS(=O)(=O)CP(=O)(O)O]

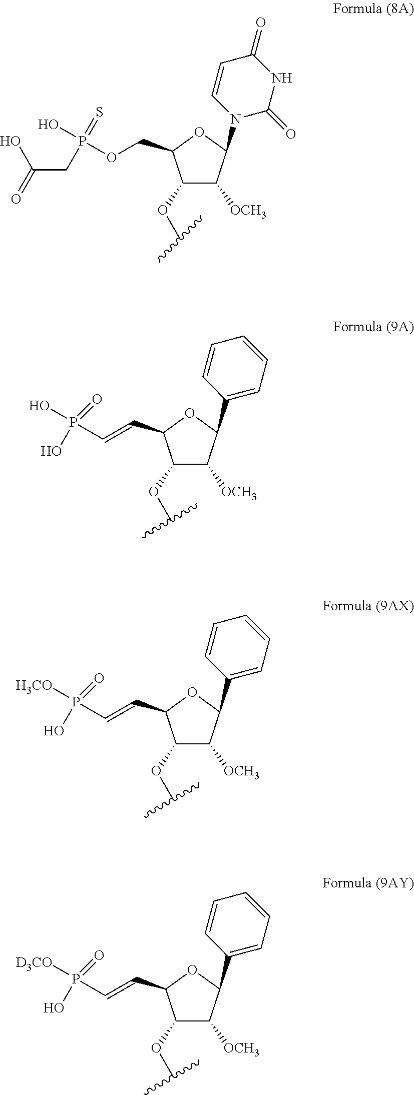

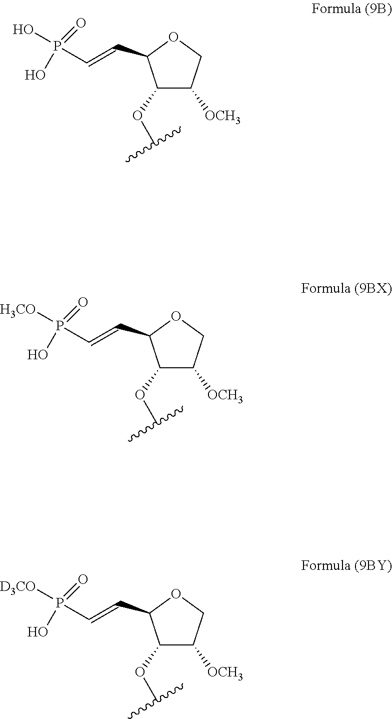

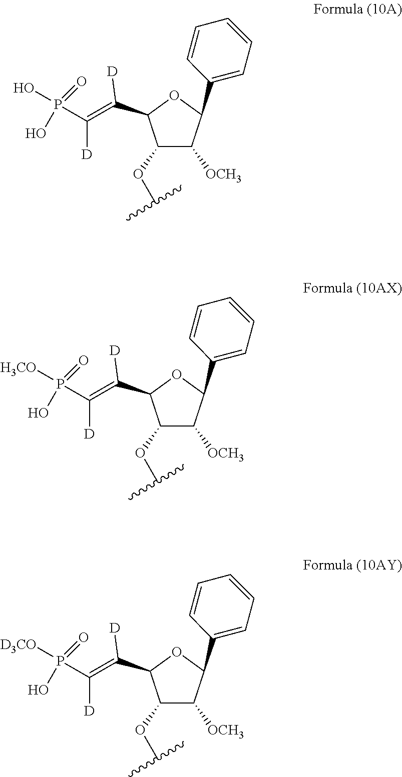

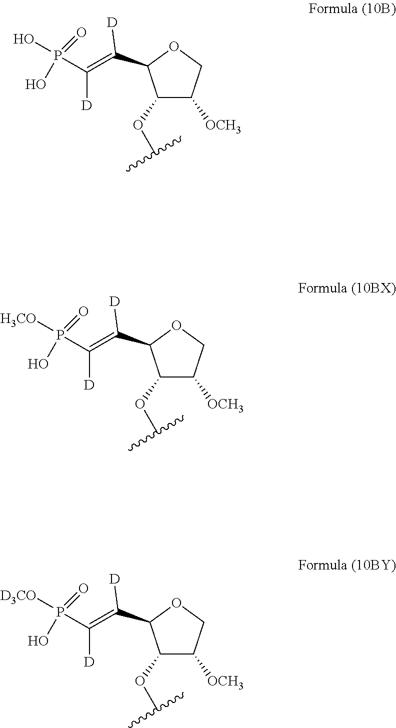

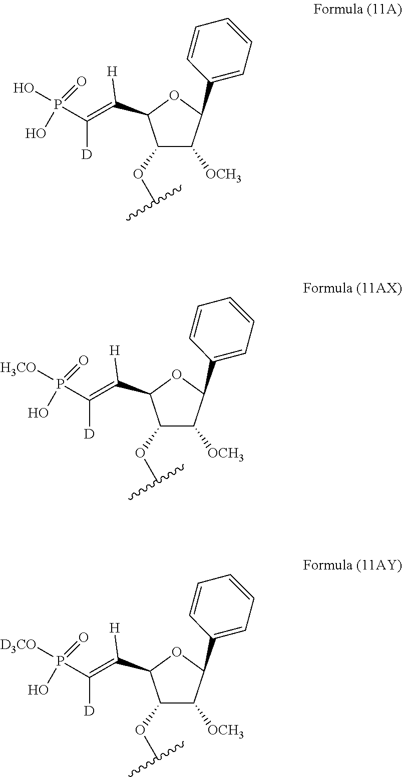

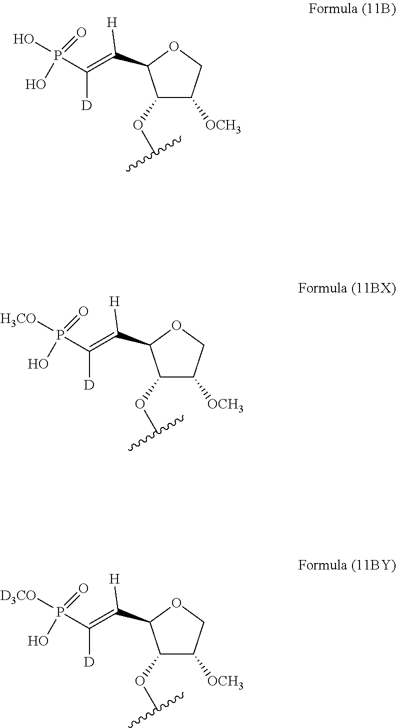

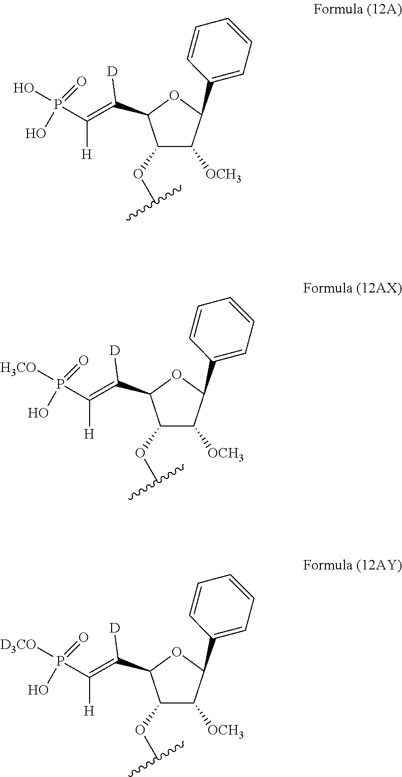

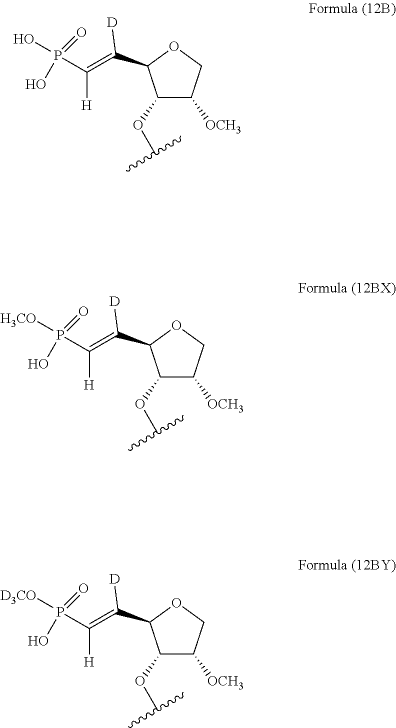

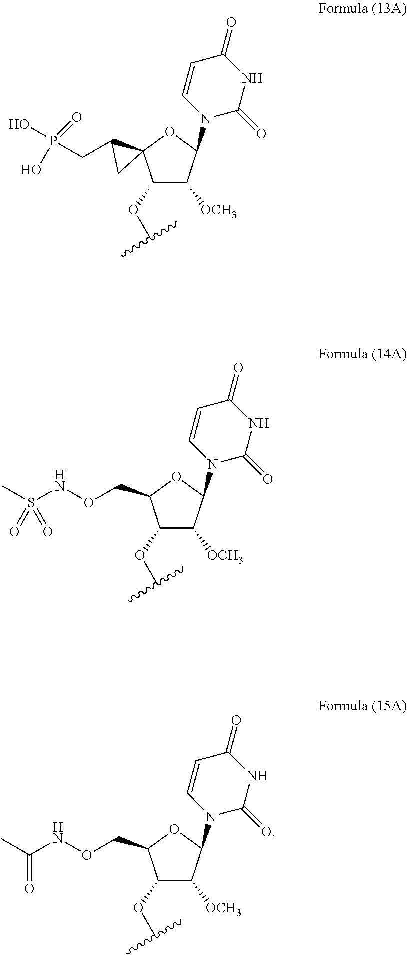

In some embodiments, any of the siNA molecules disclosed herein comprise a 5′-stabilized end cap selected from the group consisting of Formulas (21A)-(35A), Formulas (29B)-(32B), Formulas (29AX)-(32AX), Formulas (29AY)-(32AY), Formulas (29BX)-(32BX), and Formulas (29BY)-(32BY):

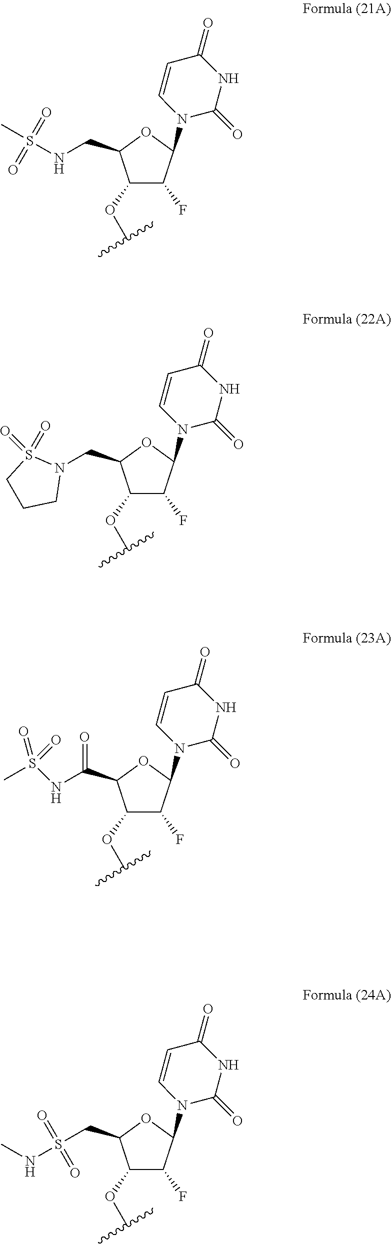 [SMILES: *O[C@H]1[C@@H](F)[C@H](n2ccc(=O)[nH]c2=O)O[C@@H]1CNS(C)(=O)=O ; *O[C@H]1[C@@H](F)[C@H](n2ccc(=O)[nH]c2=O)O[C@@H]1CN1CCCS1(=O)=O ; *O[C@H]1[C@@H](F)[C@H](n2ccc(=O)[nH]c2=O)O[C@@H]1C(=O)NS(C)(=O)=O ; *O[C@H]1[C@@H](F)[C@H](n2ccc(=O)[nH]c2=O)O[C@@H]1CS(=O)(=O)NC]

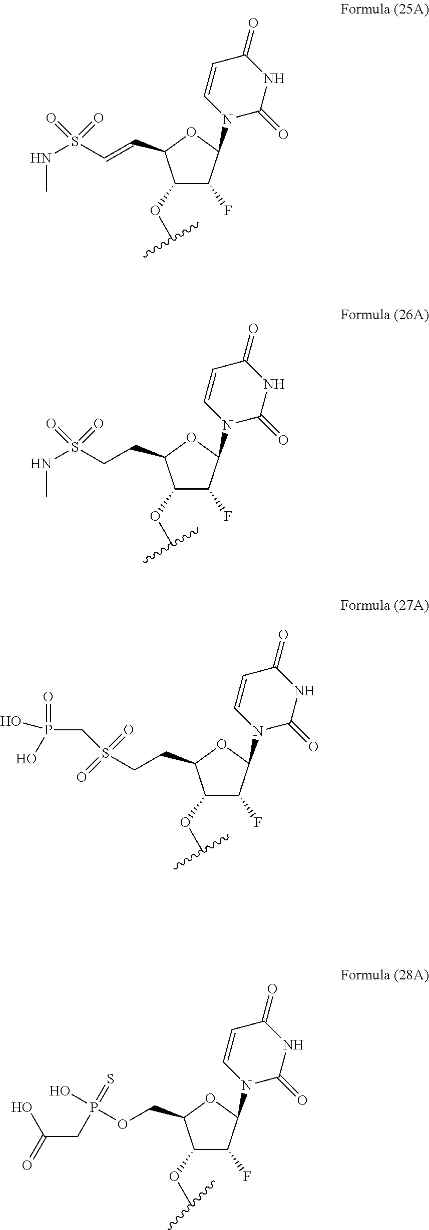 [SMILES: *O[C@H]1[C@@H](F)[C@H](n2ccc(=O)[nH]c2=O)O[C@@H]1/C=C/S(=O)(=O)NC ; *O[C@H]1[C@@H](F)[C@H](n2ccc(=O)[nH]c2=O)O[C@@H]1CCS(=O)(=O)NC ; *O[C@H]1[C@@H](F)[C@H](n2ccc(=O)[nH]c2=O)O[C@@H]1CCS(=O)(=O)CP(=O)(O)O ; *O[C@H]1[C@@H](F)[C@H](n2ccc(=O)[nH]c2=O)O[C@@H]1COP(O)(=S)CC(=O)O]

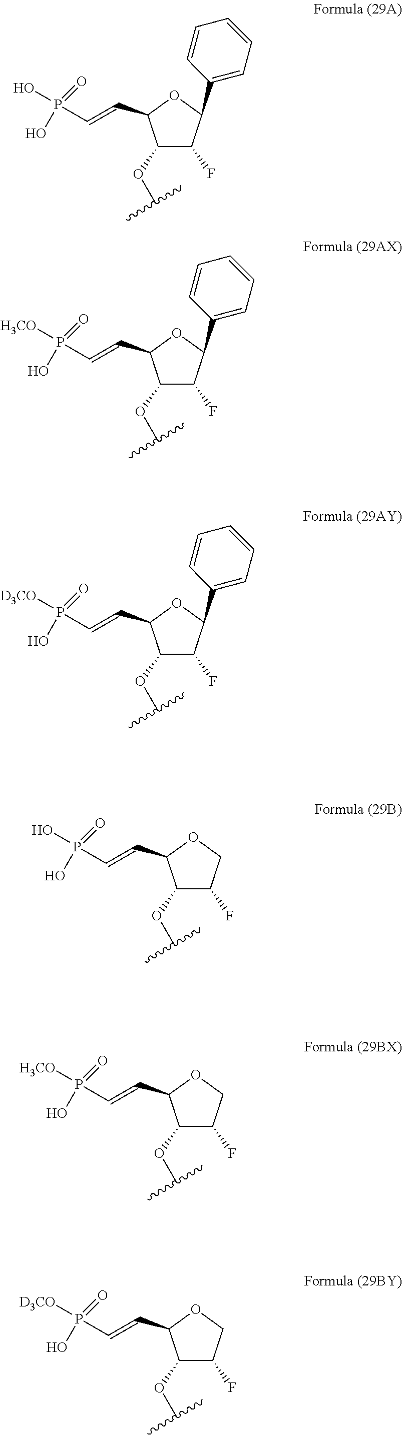

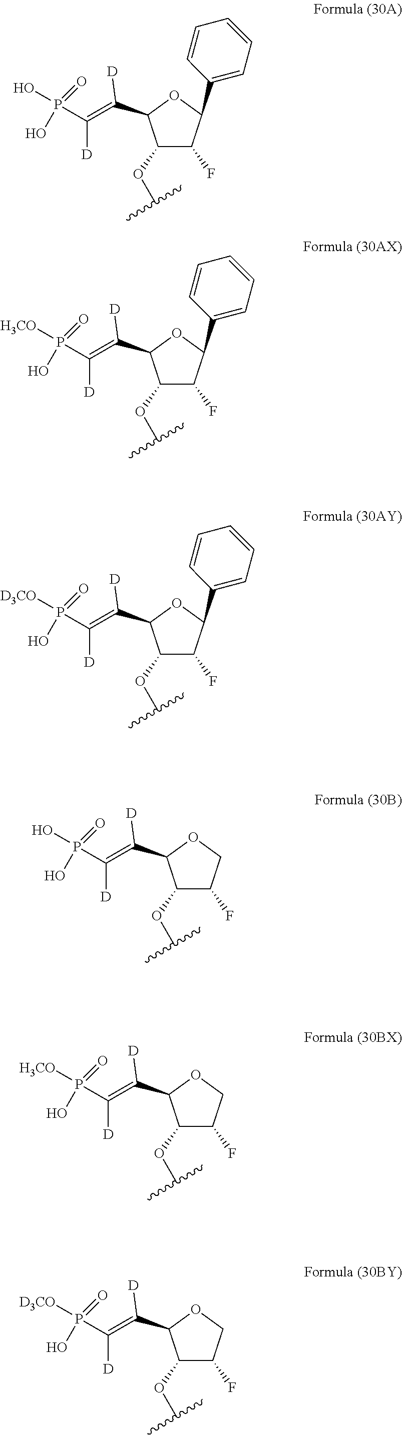

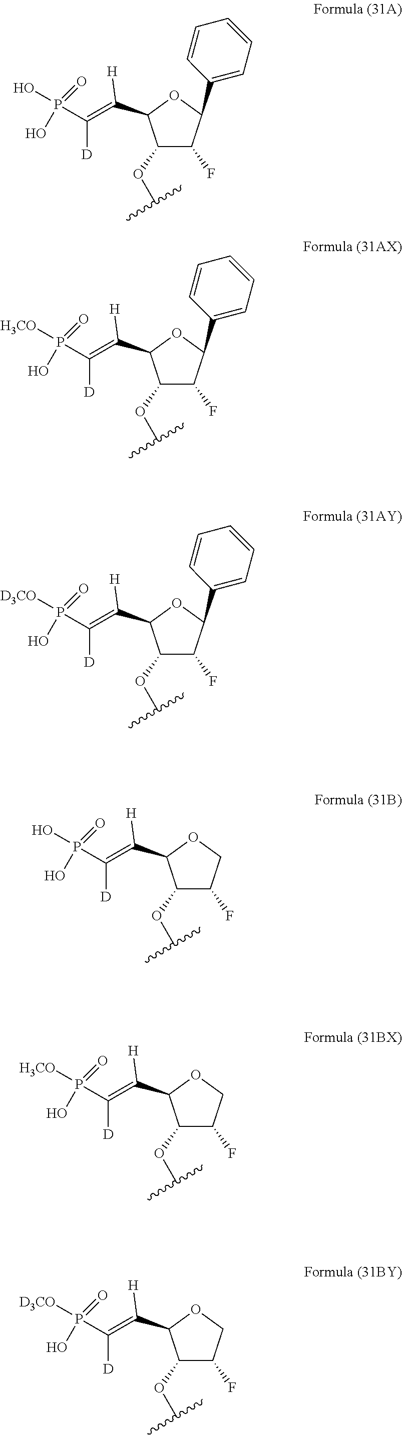

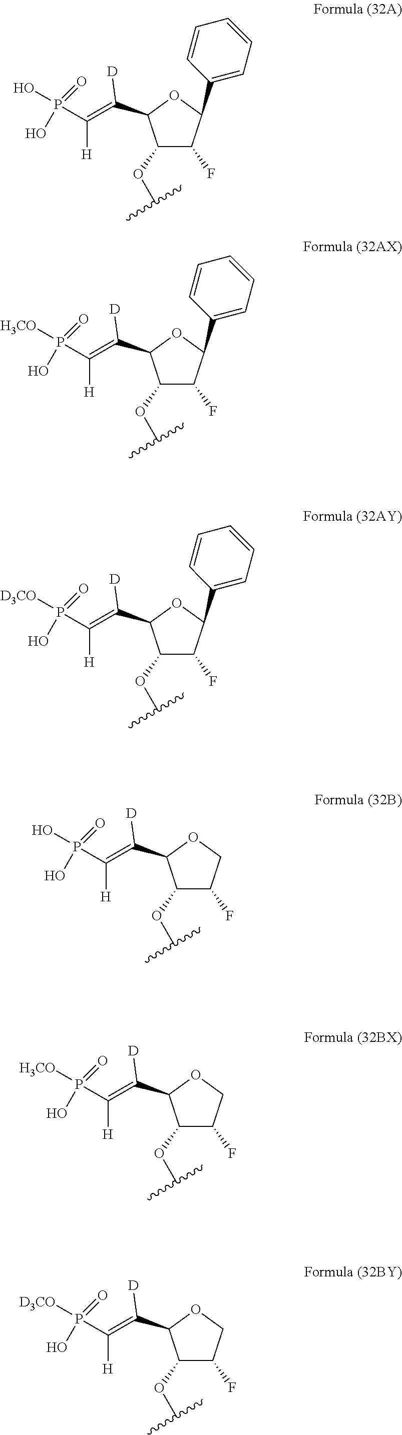

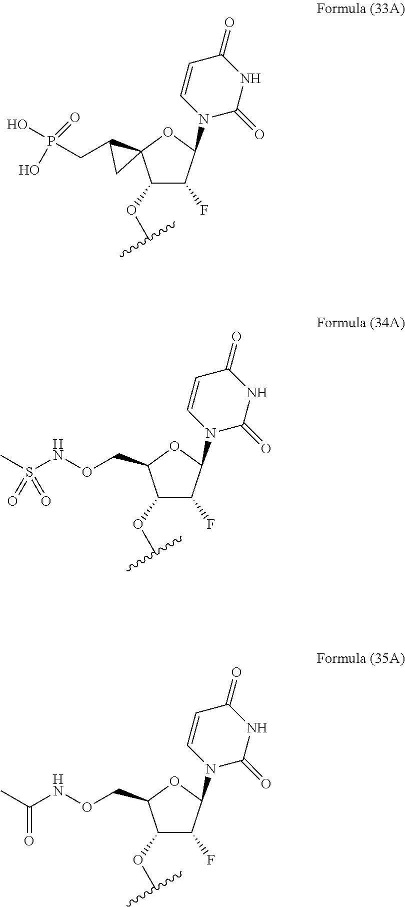

In some embodiments, any of the siNA molecules disclosed herein comprise a 5′-stabilized end cap selected from the group consisting of Formulas (71A)-(86A), Formulas (79XA)-(82XA), Formulas (79YA)-(82YA); Formula (86XA), Formula (86X′A), Formula (86Y), and Formula (86Y′):

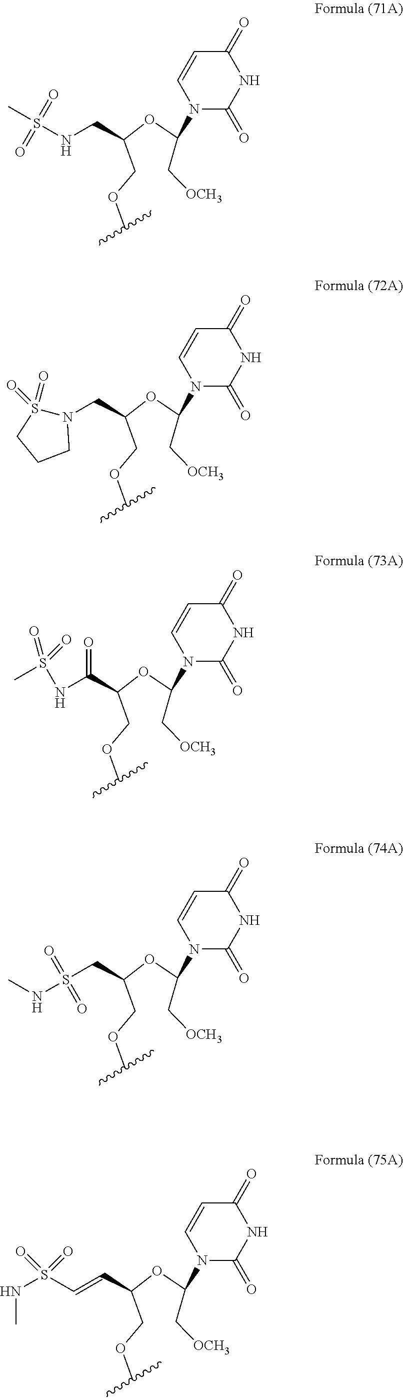 [SMILES: *OC[C@@H](CNS(C)(=O)=O)O[C@H](COC)n1ccc(=O)[nH]c1=O ; *OC[C@@H](CN1CCCS1(=O)=O)O[C@H](COC)n1ccc(=O)[nH]c1=O ; *OC[C@H](O[C@H](COC)n1ccc(=O)[nH]c1=O)C(=O)NS(C)(=O)=O ; *OC[C@@H](CS(=O)(=O)NC)O[C@H](COC)n1ccc(=O)[nH]c1=O ; *OC[C@@H](/C=C/S(=O)(=O)NC)O[C@H](COC)n1ccc(=O)[nH]c1=O]

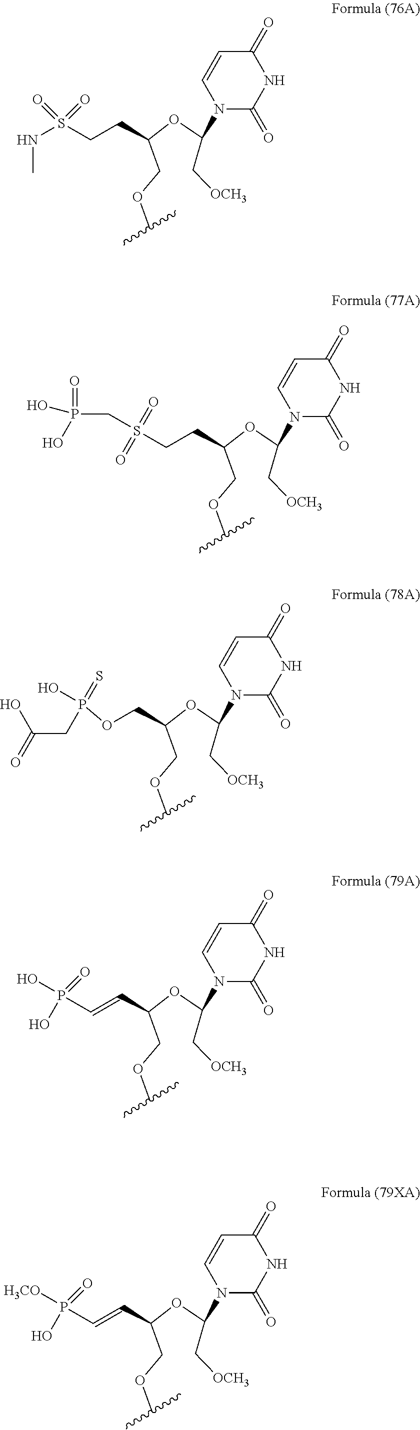 [SMILES: *OC[C@@H](CCS(=O)(=O)NC)O[C@H](COC)n1ccc(=O)[nH]c1=O ; *OC[C@@H](CCS(=O)(=O)CP(=O)(O)O)O[C@H](COC)n1ccc(=O)[nH]c1=O ; *OC[C@@H](COP(O)(=S)CC(=O)O)O[C@H](COC)n1ccc(=O)[nH]c1=O ; *OC[C@@H](/C=C/P(=O)(O)O)O[C@H](COC)n1ccc(=O)[nH]c1=O ; *OC[C@@H](/C=C/P(=O)(O)OC)O[C@H](COC)n1ccc(=O)[nH]c1=O]

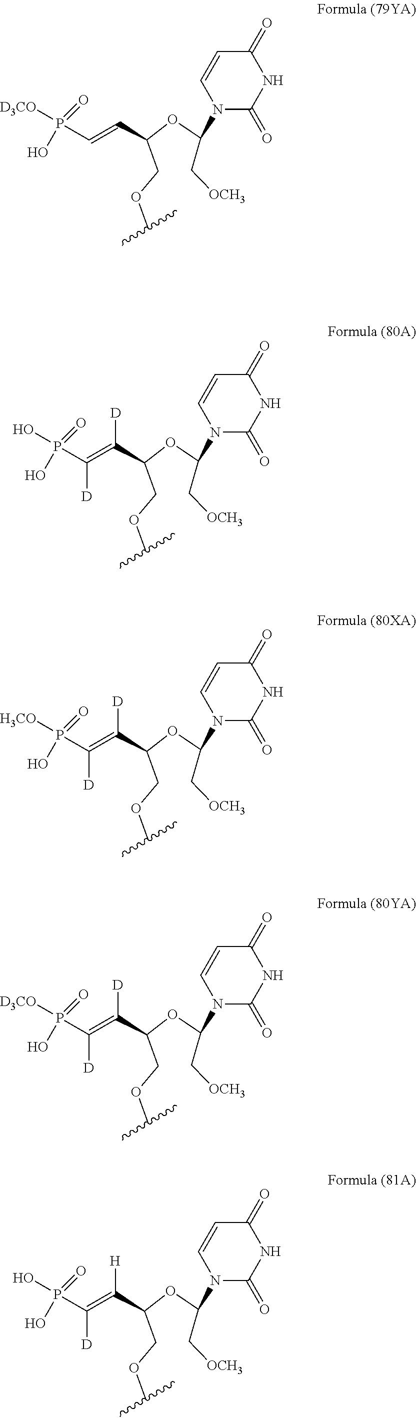

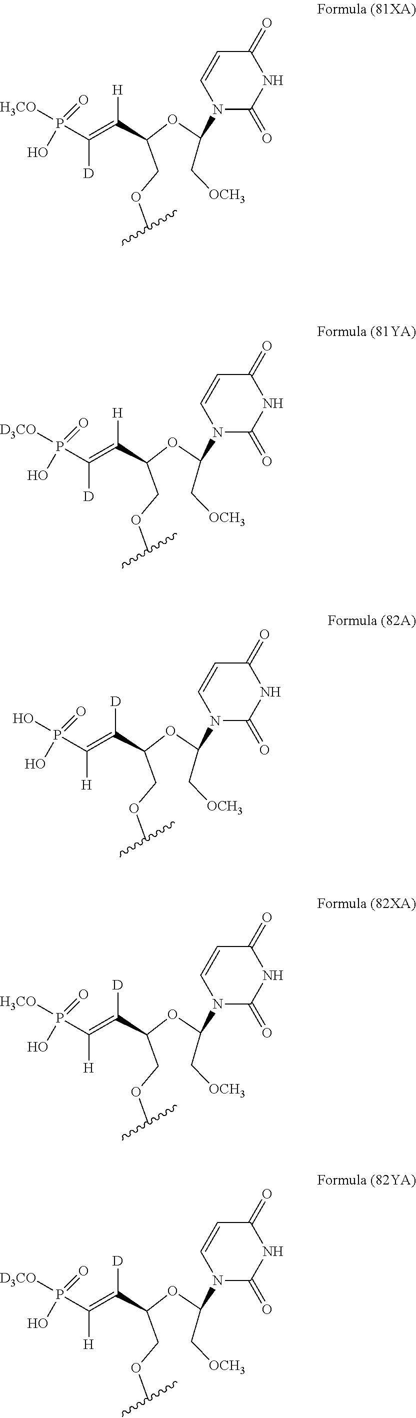

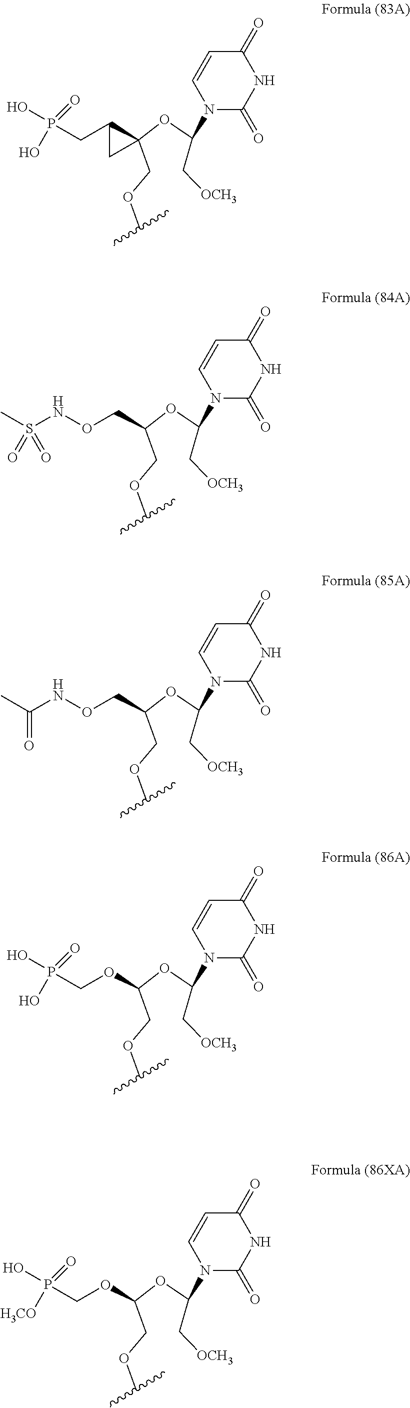

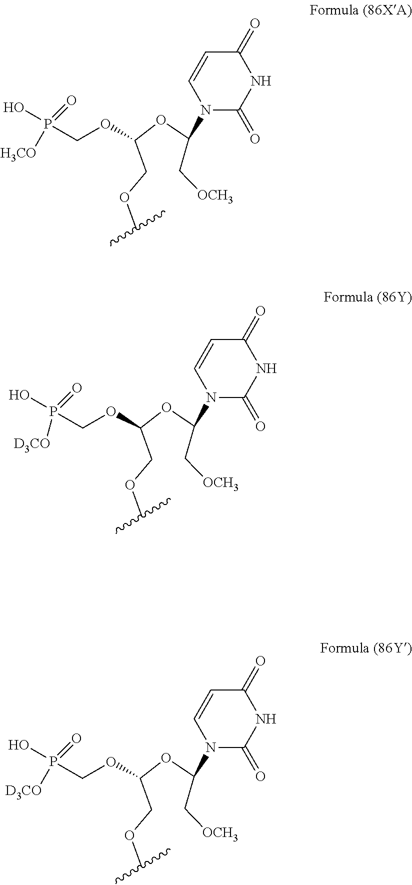

In some embodiments, any of the siNA molecules disclosed herein comprise a 5′-stabilized end cap selected from the group consisting of Formula (78A), Formula (79A), Formula (79XA), Formula (79YA), Formula (86A), Formula (86XA), and Formula (86X′A):

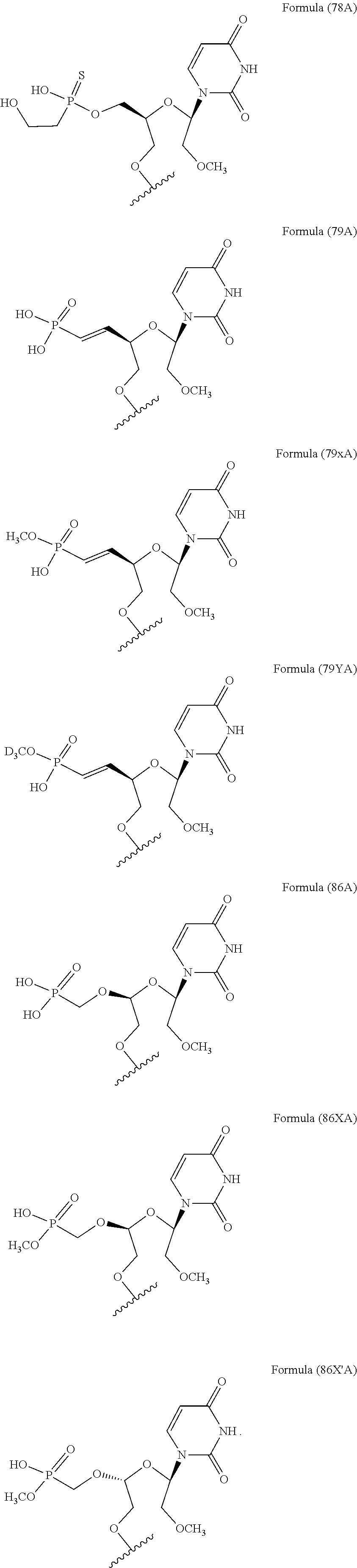

In some embodiments, the 5′-stabilized end cap is attached to the 5′ end of the antisense strand. In some embodiments, the 5′-stabilized end cap is attached to the 5′ end of the antisense strand via 1, 2, 3, 4, or 5 or more linkers. In some embodiments, the one or more linkers are independently selected from the group consisting of a phosphodiester (p or po) linker, phosphorothioate (ps) linker, mesyl phosphoramidate (Ms) linker, phosphoramidite (HEG) linker, triethylene glycol (TEG) linker, and/or phosphorodithioate linker. In some embodiments, the one or more linkers are independently selected from the group consisting of p-(PS)2, (PS)2-p-TEG-p, (PS)2-p-HEG-p, and (PS)2-p-(HEG-p)2.

As indicated above, the present disclosure provides compositions comprising any of the siNA molecules, sense strands, antisense strands, first nucleotide sequences, or second nucleotide sequences described herein. The disclosed siNA and compositions thereof can be used in the treatment of various diseases and conditions (e.g., viral diseases, liver disease, etc.). Linker

In some embodiments, any of the siRNAs, sense strands, first nucleotide sequences, antisense strands, and/or second nucleotide sequences disclosed herein comprise 1, 2, 3, 4, 5, 6, 7, 8, 9, 10, 11, 12, 13, 14, 15, 16, 17, 18, 19, 20, 21, 22, 23 or more internucleoside linkers. In some embodiments, 1, 2, 3, 4, 5, 6, 7, 8, 9, 10 or more internucleoside linkers are independently selected from the group consisting of a phosphodiester (p or po) linker, phosphorothioate (ps) linker, mesyl phosphoramidate (Ms) linker, or phosphorodithioate linker.

In some embodiments, any of the siRNAs, sense strands, first nucleotide sequences, antisense strands, and/or second nucleotide sequences disclosed herein further comprise 1, 2, 3, 4 or more linkers that attach a conjugated moiety, phosphorylation blocker, and/or 5′ end cap to the siRNA, sense strand, first nucleotide sequence, antisense strand, and/or second nucleotide sequences. In some embodiments, the 1, 2, 3, 4 or more linkers are independently selected from the group consisting of a phosphodiester (p or po) linker, phosphorothioate (ps) linker, mesyl phosphoramidate (Ms), phosphoramidite (HEG) linker, triethylene glycol (TEG) linker, and/or phosphorodithioate linker. In some embodiments, the one or more linkers are independently selected from the group consisting of p-(PS)2, (PS)2-p-TEG-p, (PS)2-p-HEG-p, and (PS)2-p-(HEG-p)2.

Exemplary siNA

As noted above, the siNA disclosed herein may comprise a modified nucleotide, such as the 2′-fluoro nucleotide fB, fN, or 4(4nh)Q. Other 2′-fluoro nucleotides, such as f2P, f4P, and fX may be incorporated into the disclosed siNA as well. A siNA comprising a disclosed 2′-fluoro nucleotide (e.g., fB, fN, or 4(4nh)Q and bolded in the Table) may comprise one or more of the disclosed 2′-fluoro nucleotides and the one or more 2′-fluoro nucleotides may be present in the sense strand or the antisense strand or both. Table 1 shows exemplary siNA comprising these 2′-fluoro nucleotides.

TABLE 1
siNA Comprising 2′-Fluoro Nucleotides

| Name | SS/AS 5′ to 3′ |
|---|---|
| ds-siNA-001 | mGpsmUpsmGmGfUmGfGfAfCmUmUmCmUmCmUmCmAmAmU |
| | (SEQ ID NO: 1) |
| | mApsf2PpsmUmGmAfGmAmGmAmAmGmUmCfCmAfCmCmAmCpsmGp |
| | smA |
| | (SEQ ID NO: 2) |
| ds-siNA-002 | mGpsmUpsmGmGfUmGfGfAfCmUmUmCmUmCmUmCmAmAmU |
| | (SEQ ID NO: 3) |
| | mApsfUpsmUmGmAfGmAmGmAmAmGmUmCf4PmAfCmCmAmCpsmGp |
| | smA |
| | (SEQ ID NO: 4) |
| ds-siNA-003 | mGpsmUpsmGmGfUmGfGfAfCmUmUmCmUmCmUmCmAmAmU |
| | (SEQ ID NO: 5) |
| | mApsfUpsmUmGmAfGmAmGmAmAmGmUmCfCmAf2PmCmAmCpsmGp |
| | smA |
| | (SEQ ID NO: 6) |
| ds-siNA-004 | mGpsmUpsmGmGfUmGfGfAfCmUmUmCmUmCmUmCmAmAmU |
| | (SEQ ID NO: 7) |
| | mApsfUpsmUmGmAfGmAmGmAmAmGmUmCf2PmAf2PmCmAmCpsmG |
| | psmA |
| | (SEQ ID NO: 8) |
| ds-siNA-005 | mGpsmUpsmGmGfUmGfGfAfCmUmUmCmUmCmUmCmAmAmU |
| | (SEQ ID NO: 9) |
| | mApsfBpsmUmGmAfGmAmGmAmAmGmUmCfCmAfCmCmAmCpsmGps |
| | mA |
| | (SEQ ID NO: 10) |
| ds-siNA-006 | mGpsmUpsmGmGfUmGfGfAfCmUmUmCmUmCmUmCmAmAmU |
| | (SEQ ID NO: 11) |
| | mApsfUpsmUmGmAfGmAmGmAmAmGmUmCfBmAfCmCmAmCpsmGps |
| | mA |
| | (SEQ ID NO: 12) |
| ds-siNA-007 | mGpsmUpsmGmGfUmGfGfAfCmUmUmCmUmCmUmCmAmAmU |
| | (SEQ ID NO: 13) |
| | mApsfUpsmUmGmAfGmAmGmAmAmGmUmCfBmAfBmCmAmCpsmGps |
| | mA |
| | (SEQ ID NO: 14) |
| ds-siNA-008 | mGpsmUpsmGmGfUmGfGfAfCmUmUmCmUmCmUmCmAmAmU |
| | (SEQ ID NO: 15) |
| | mApsfUpsmUmGmAfGmAmGmAmAmGmUmCf2PmAfCmCmAmCpsmGp |
| | smA |
| | (SEQ ID NO: 16) |
| ds-siNA-023 | 5′- |
| | mCpsmCpsmGmUfGmUfGf(4nh)QfAmCmUmUmCmGmCmUmUmCmA-p- |
| | (ps)2-GalNAc4 |
| | (SEQ ID NO: 63) |
| | 3′- |
| | mCpsmUpsmGmGfCmAmCfAmCmGmUmGmAfAmGmCfGmAmApsfGpsd |
| | 2vd3U-5′ |
| | (SEQ ID NO: 64) |
| ds-siNA-024 | 5 ′ -mCpsmCpsmGmUfGmUfGfCfAmCmUmUmCmGmCmUmUmCm A-p- |
| | (ps)2-GalNAc4 |
| | (SEQ ID NO: 65) |
| | 3′- |
| | mCpsmUpsmGmGfCmAmCfAmCmGmUmGmAfAmGf(4nh)QfGmAmApsf |
| | Gpsd2vd3U-5′ |
| | (SEQ ID NO: 66) |
| ds-siNA-025 | 5 ′ -mCpsmCpsmGmUfGmUfGfCfAmCmUmUmCmGmCmUmUmCm A-p- |
| | (ps)2GalNAc4 |
| | (SEQ ID NO: 67) |
| | 3′- |
| | mCpsmUpsmGmGf(4nh)QmAmCfAmCmGmUmGmAfAmGmCfGmAmApsf |
| | Gpsd2vd3U-5′ |
| | (SEQ ID NO: 68) |

mX = 2′-O-methyl nucleotide;
fX = 2′-fluoro nucleotide;
5dcd3X = nucleotide of Formula 17;
5dfX = nucleotide of Formula 16;
vX= 5′ vinyl phosphonate nucleotide;
d2vX = deuterated 5′ vinyl phosphonate nucleotide;
vmX = 5′ vinyl phosphonate, 2′-O-methyl nucleotide;
; f4P = 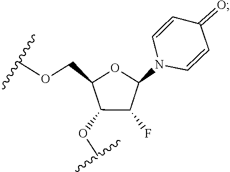 [SMILES: *OC[C@H]1O[C@@H](n2ccc(=O)cc2)[C@H](F)[C@@H]1O*]
f2P = 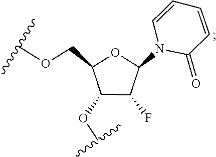 [SMILES: *OC[C@H]1O[C@@H](n2ccccc2=O)[C@H](F)[C@@H]1O*]
fB = 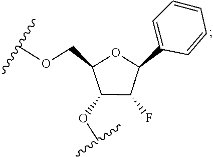 [SMILES: *OC[C@H]1O[C@@H](c2ccccc2)[C@H](F)[C@@H]1O*]
f(4nh)Q = 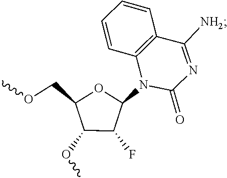
ps = phosphorothioate linkage;
X is a nucleobase (e.g. A, G, C, U or T)

Additionally or alternatively, the disclosed siNA may also incorporate a novel nucleotide phosphate mimic (e.g., omeco-d3U, 4hU, v-mun, c2o-4h, omeco-mun, d2vmA, coc-4 h, 4H-VP nucleotide). Table 2 shows exemplary siNA comprising these nucleotide phosphate mimics. A siNA comprising a disclosed novel phosphate mimic (e.g., omeco-d3U, 4hU, v-mun, c2o-4h, omeco-mun, coc-4h, or d2vmA and bolded in the Table) may comprise one or more of the disclosed novel phosphate mimic and the one or more novel phosphate mimics may be present in the sense strand or the antisense strand or both.

TABLE 2
siNA Comprising Nucleotide Phosphate Mimics

| Name | SS/AS 5′ to 3′ |
|---|---|
| ds-siNA-009 | mCpsmCpsmGmUfGmUfGfCfAmCmUmUmCmGmCmUmUmCmAp-ps2- |
| | GalNAc4 |
| | (SEQ ID NO: 17) |
| | omeco- |
| | d3UpsfGpsmAmAfGmCmGfAmAmGmUmGmCfAmCmAfCmGmGpsmUps |
| | mC |
| | (SEQ ID NO: 18) |
| ds-siNA-010 | mCpsmCpsmGmUfGmUfGfCfAmCmUmUmCmGmCmUmUmCmAp-ps2- |
| | GalNAc4 |
| | (SEQ ID NO: 19) |
| | 4hUpsfGpsmAmAfGmCmGfAmAmGmUmGmCfAmCmAfCmGmGpsmUps |
| | mC |
| | (SEQ ID NO: 20) |
| ds-siNA-011 | mCpsmCpsmGmUfGmUfGfCfAmCmUmUmCmGmCmUmUmCmAp-ps2- |
| | GalNAc4 |
| | (SEQ ID NO: 21) |
| | d2vd3UpsfGpsmAmAfGmCmGfAmAmGmUmGmCfAmCmAfCmGmGpsm |
| | UpsmC |
| | (SEQ ID NO: 22) |
| ds-siNA-012 | mCpsmCpsmGmUfGmUfGfCfAmCmUmUmCmGmCmUmUmCmA-p-ps2- |
| | GalNAc4 |
| | (SEQ ID NO: 23) |
| | v- |
| | munUpsfGpsmAmAfGmCmGfAmAmGmUmGmCfAmCmAfCmGmGpsmU |
| | psmC |
| | (SEQ ID NO: 24) |
| ds-siNA-013 | mCpsmCpsmGmUfGmUfGfCfAmCmUmUmCmGmCmUmUmCmA-p-ps2- |
| | GalNAc4 |
| | (SEQ ID NO: 25) |
| | c2o- |
| | 4hUpsfGpsmAmAfGmCmGfAmAmGmUmGmCfAmCmAfCmGmGpsmUps |
| | mC |
| | (SEQ ID NO: 26) |
| ds-siNA-014 | mCpsmCpsmGmUfGmUfGfCfAmCmUmUmCmGmCmUmUmCmA-p-ps2- |
| | GalNAc4 |
| | (SEQ ID NO: 27) |
| | omeco- |
| | munUpsfGpsmAmAfGmCmGfAmAmGmUmGmCfAmCmAfCmGmGpsmU |
| | psmC |
| | (SEQ ID NO: 28) |
| ds-siNA-015 | mCpsmCpsmGmUfGmUfGfCfAmCmUmUmCmGmCmUmUmCmA-p-ps2- |
| | GalNAc4 |
| | (SEQ ID NO: 29) |
| | omeco- |
| | munUpsfGpsmAmAfGmCmGfAmAmGmUmGmCfAmCmAfCmGmGpsmU |
| | psmC |
| | (SEQ ID NO: 30) |
| ds-siNA-016 | mGpsmUpsmGmGfUmGfGfAfCmUmUmCmUmCmUmCmAmAmU-p-ps2- |
| | GalNAc4 |
| | (SEQ ID NO: 33) |
| | d2vmApsfUpsmUmGmAfGmAmGmAmAmGmUmCfCmAf2PmCmAmCpsm |
| | GpsmA |
| | (SEQ ID NO: 34) |
| ds-siNA-050 | mCpsmCpsmGmUfGmUfGfCfAmCmUmUmCmGmCmUmUmCmA-p-(ps)2- |
| | GalNAc4 |
| | (SEQ ID NO: 111) |
| | coc-4hUpsfGpsmAmAfGmCmGfAmAmGmUmGmCfAmCmAfCmGmGpsm |
| | UpsmC |
| | (SEQ ID NO: 112) |

mX = 2′-O-methyl nucleotide;
fX = 2′-fluoro nucleotide;
5dcd3X = nucleotide of Formula 17;
5dfX = nucleotide of Formula 16;
vX= 5′ vinyl phosphonate nucleotide;
d2vX = deuterated 5′ vinyl phosphonate nucleotide;
vmX = 5′ vinyl phosphonate, 2′-O-methyl nucleotide;
omeco-d3U = 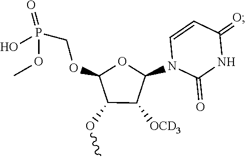
4hU = 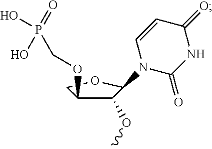
d2vd3U = 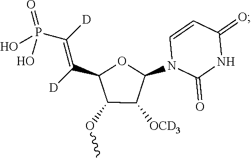
v-munU = 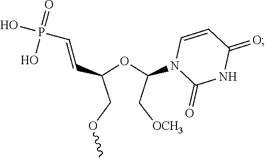
c2o-4hU = 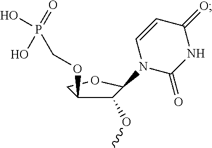
coc-4hU = 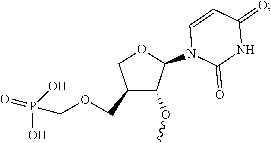
omeco-munU = 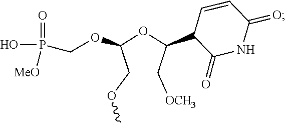
ps = phosphorothioate linkage;
X is a nucleobase (e.g. A, G, C, U or T)

Additionally or alternatively, the disclosed siNA may also incorporate a novel unlocked nucleotide monomers. These novel unlocked nucleotides may have of structure of

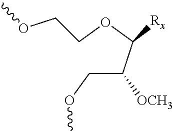

(wherein Rₓ is a nucleobase, aryl, heteroaryl, or H) or, more specifically,

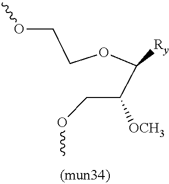

wherein R_{y} is a nucleobase. These unlocked nucleotides are distinct from unlock nucleic acids (UNA) known in the art in which the 2′ to 3′ bond is missing

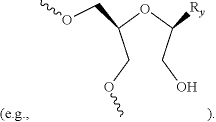

Table 3 shows exemplary siNA comprising these unlocked nucleotides. A siNA comprising a 3′,4′ UNA (e.g., mun34) may comprise one or more of the disclosed 3′, 4′ UNAs and the one or more 3′, 4′ UNAs may be present in the sense strand or the antisense strand or both.

TABLE 3
siNA Comprising Modified Unlocked Nucleotides

| Name | SS/AS (5′ to 3′) |
|---|---|
| ds-siNA-017 | mCpsmCpsmGmUfGmUfGfCfAmCmUmUmCmGmCmUmUmCmA |
| | (SEQ ID NO: 35) |
| | mUpsfGpsmAmAfGmun34CmGfAmAmGmUmGmCfAmCmAfCmGmGpsmU |
| | psmC |
| | (SEQ ID NO: 36) |
| ds-siNA-018 | mCpsmCpsmGmUfGmUfGfCfAmCmUmUmCmGmCmUmUmCmA |
| | (SEQ ID NO: 37) |
| | mUpsfGpsmAmAfGmCmun34GfAmAmGmUmGmCfAmCmAfCmGmGpsmU |
| | psmC |
| | (SEQ ID NO: 38) |
| ds-siNA-019 | mCpsmCpsmGmUfGmUfGfCfAmCmUmUmCmGmCmUmUmCmA-p-ps2- |
| | GalNAc4 |
| | (SEQ ID NO: 39) |
| | d2vd3UpsfGpsmAmAfGmCmun34GfAmAmGmUmGmCfAmCmAfCmGmGps |
| | mUpsmC |
| | (SEQ ID NO: 40) |
| ds-siNA-034 | mCpsmCpsmun34GmUfGmUfGfCfAmCmUmUmCmGmCmUmUmCmA |
| | (SEQ ID NO: 93) |
| | psmUpsfGmAmAfGmCmGfAmAmGmUmGmCfAmCmAfCmGpsmGpsmUmC |
| | (SEQ ID NO: 94) |
| ds-siNA-035 | mCpsmCpsmGmun34UfGmUfGfCfAmCmUmUmCmGmCmUmUmCmA |
| | (SEQ ID NO: 95) |
| | psmUpsfGmAmAfGmCmGfAmAmGmUmGmCfAmCmAfCmGpsmGpsmUmC |
| | (SEQ ID NO: 94) |
| ds-siNA-036 | mCpsmCpsmGmUfGmun34UfGfCfAmCmUmUmCmGmCmUmUmCmA |
| | (SEQ ID NO: 96) |
| | psmUpsfGmAmAfGmCmGfAmAmGmUmGmCfAmCmAfCmGpsmGpsmUmC |
| | (SEQ ID NO: 94) |
| ds-siNA-037 | mCpsmCpsmGmUfGmUfGfCfAmun34CmUmUmCmGmCmUmUmCmA |
| | (SEQ ID NO: 97) |
| | psmUpsfGmAmAfGmCmGfAmAmGmUmGmCfAmCmAfCmGpsmGpsmUmC |
| | (SEQ ID NO: 94) |
| ds-siNA-038 | mCpsmCpsmGmUfGmUfGfCfAmCmun34UmUmCmGmCmUmUmCmA |
| | (SEQ ID NO: 98) |
| | psmUpsfGmAmAfGmCmGfAmAmGmUmGmCfAmCmAfCmGpsmGpsmUmC |
| | (SEQ ID NO: 94) |
| ds-siNA-039 | mCpsmCpsmGmUfGmUfGfCfAmCmUmun34UmCmGmCmUmUmCmA |
| | (SEQ ID NO: 99) |
| | psmUpsfGmAmAfGmCmGfAmAmGmUmGmCfAmCmAfCmGpsmGpsmUmC |
| | (SEQ ID NO: 94) |
| ds-siNA-040 | mCpsmCpsmGmUfGmUfGfCfAmCmUmUmun34CmGmCmUmUmCmA |
| | (SEQ ID NO: 100) |
| | psmUpsfGmAmAfGmCmGfAmAmGmUmGmCfAmCmAfCmGpsmGpsmUmC |
| | (SEQ ID NO: 94) |
| ds-siNA-041 | mCpsmCpsmGmUfGmUfGfCfAmCmUmUmCmun34GmCmUmUmCmA |
| | (SEQ ID NO: 101) |
| | psmUpsfGmAmAfGmCmGfAmAmGmUmGmCfAmCmAfCmGpsmGpsmUmC |
| | (SEQ ID NO: 94) |
| ds-siNA-042 | mCpsmCpsmGmUfGmUfGfCfAmCmUmUmCmGmun34CmUmUmCmA |
| | (SEQ ID NO: 102) |
| | psmUpsfGmAmAfGmCmGfAmAmGmUmGmCfAmCmAfCmGpsmGpsmUmC |
| | (SEQ ID NO: 94) |
| ds-siNA-043 | mCpsmCpsmGmUfGmUfGfCfAmCmUmUmCmGmCmun34UmUmCmA |
| | (SEQ ID NO: 103) |
| | psmUpsfGmAmAfGmCmGfAmAmGmUmGmCfAmCmAfCmGpsmGpsmUmC |
| | (SEQ ID NO: 94) |
| ds-siNA-044 | mCpsmCpsmGmUfGmUfGfCfAmCmUmUmCmGmCmUmun34UmCmA |
| | (SEQ ID NO: 104) |
| | psmUpsfGmAmAfGmCmGfAmAmGmUmGmCfAmCmAfCmGpsmGpsmUmC |
| | (SEQ ID NO: 94) |
| ds-siNA-045 | mCpsmCpsmGmUfGmUfGfCfAmCmUmUmCmGmCmUmUmun34CmA |
| | (SEQ ID NO: 105) |
| | psmUpsfGmAmAfGmCmGfAmAmGmUmGmCfAmCmAfCmGpsmGpsmUmC |
| | (SEQ ID NO: 94) |

mX = 2′-O-methyl nucleotide;
fX = 2′-fluoro nucleotide;
5dcd3X = nucleotide of Formula 17;
5dfX = nucleotide of Formula 16;
vX = 5′ vinyl phosphonate nucleotide;
d2vX = deuterated 5′ vinyl phosphonate nucleotide;
vmX = 5′ vinyl phosphonate, 2′-O-methyl nucleotide;
d2vd3U = 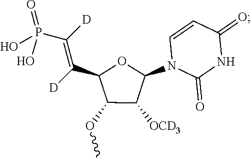
mun34C = 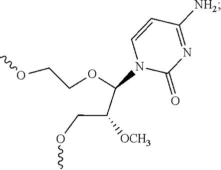
mun34G = 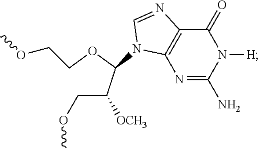
unC = 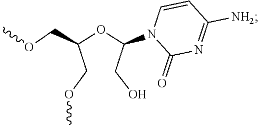
unG = 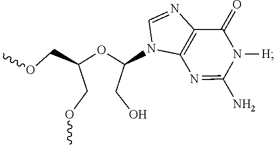
ps = phosphorothioate linkage;
X is a nucleobase (e.g. A, G, C, U or T)

Additionally or alternatively, the disclosed siNA may also incorporate 1 or more mesyl phosphoroamidate internucleoside linkages. The mesyl phosphoroamidate internucleoside linkage (also known as “yp”) may have the structure of

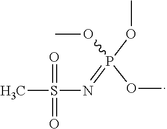

Table 4 shows exemplary siNA comprising these mesyl phosphoroamidate internucleoside linkages. A siNA comprising mesyl phosphoroamidate internucleoside linkage (denoted “yp” and bolded in the Table) may comprise one or more yp linkages and the one or more yp linkages may be present in the sense strand or the antisense strand or both.

TABLE 4
siNA Comprising Mesyl Phosphoroamidate Internucleoside Linkages

| Name | SS/AS (5′ to 3′) |
|---|---|
| ds-siNA-020 | 5′-mGpsmUpsmGmGfUmGfGfAfCmUmUmCmUmCmUmCm Am AmU-p- |
| | (ps)2-GalNAc4 |
| | (SEQ ID NO: 69) |
| | 3′- |
| | mApsmGpsmCmAmCfCmAfCmCmUmGmAmAmGmAfGmAmGmUypfUypm |
| | A-5′ |
| | (SEQ ID NO: 70) |
| ds-siNA-021 | 5′-mGpsmUpsmGmGfUmGfGfAfCmUmUmCmUmCmUmCm Am AmU-p- |
| | (ps)2-GalNAc4 |
| | (SEQ ID NO: 71) |
| | 3′- |
| | mAypmGypmCmAmCfCmAfCmCmUmGmAmAmGmAfGmAmGmUpsfUpsm |
| | A-5′ |
| | (SEQ ID NO: 72) |
| ds-siNA-022 | 5′-mGpsmUpsmGmGfUmGfGfAfCmUmUmCmUmCmUmCm Am AmU-p- |
| | (ps)2-GalNAc4 |
| | (SEQ ID NO: 73) |
| | 3′- |
| | mAypmGypmCmAmCfCmAfCmCmUmGmAmAmGmAfGmAmGmUypfUyp |
| | mA-5′ |
| | (SEQ ID NO: 74) |

mX = 2′-O-methyl nucleotide;
fX = 2′-fluoro nucleotide;
ps = phosphorothioate linkage;
X is a nucleobase (e.g. A, G, C, U or T);
yp =

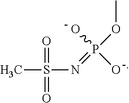

Additionally or alternatively, the disclosed siNA may also incorporate a novel monomer referred to herein as “apN,” which has a structure of

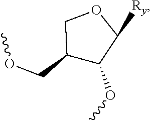

wherein R_{y} represents a nucleobase (e.g., U, A, G, T, C), and in some embodiments, the apN may be an “apU,” which has a structure of

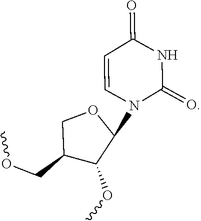

Table 5 shows exemplary siNA comprising these modified nucleotides. A siNA comprising an apU nucleotide (denoted “aU” and bolded in the Table) may comprise one or more apU nucleotides and the one or more apU nucleotides may be present in the sense strand or the antisense strand or both.

TABLE 5
siNA Comprising Modified apU Nucleotides

| Name | SS/AS (5′ to 3′) |
|---|---|
| ds-siNA-026 | mCpsmCpsmGaUfGmUfGfCfAmCmUmUmCmGmCmUmUmCmA-p-(ps)2- |
| | GalNAc4 |
| | (SEQ ID NO: 77) |
| | d2vd3UpsfGpsmAmAfGmCmGfAmAmGmUmGmCfAmCmAfCmGmGpsmUp |
| | smC |
| | (SEQ ID NO: 78) |
| ds-siNA-027 | mCpsmCpsmGmUfGaUfGfCfAmCmUmUmCmGmCmUmUmCmA-p-(ps)2- |
| | GalNAc4 |
| | (SEQ ID NO: 79) |
| | d2vd3UpsfGpsmAmAfGmCmGfAmAmGmUmGmCfAmCmAfCmGmGpsmUp |
| | smC |
| | (SEQ ID NO: 80) |
| ds-siNA-028 | mCpsmCpsmGmUfGmUfGfCfAmCmUaUmCmGmCmUmUmCmA-p-(ps)2- |
| | GalNAc4 |
| | (SEQ ID NO: 81) |
| | d2vd3UpsfGpsmAmAfGmCmGfAmAmGmUmGmCfAmCmAfCmGmGpsmUp |
| | smC |
| | (SEQ ID NO: 82) |
| ds-siNA-029 | mCpsmCpsmGmUfGmUfGfCfAmCmUmUmCmGmCaUmUmCmA-p-(ps)2- |
| | GalNAc4 |
| | (SEQ ID NO: 83) |
| | d2vd3UpsfGpsmAmAfGmCmGfAmAmGmUmGmCfAmCmAfCmGmGpsmUp |
| | smC |
| | (SEQ ID NO: 84) |
| ds-siNA-030 | mCpsmCpsmGmUfGmUfGfCfAmCmUmUmCmGmCmUaUmCmA-p-(ps)2- |
| | GalNAc4 |
| | (SEQ ID NO: 85) |
| | d2vd3UpsfGpsmAmAfGmCmGfAmAmGmUmGmCfAmCmAfCmGmGpsmUp |
| | smC |
| | (SEQ ID NO: 86) |
| ds-siNA-031 | mCpsmCpsmGmUfGmUfGfCfAmCmUmUmCmGmCmUmUmCmA-p-(ps)2- |
| | GalNAc4 |
| | (SEQ ID NO: 87) |
| | d2vd3UpsfGpsmAmAfGmCmGfAmAmGaUmGmCfAmCmAfCmGmGpsmUps |
| | mC |
| | (SEQ ID NO: 88) |
| ds-siNA-032 | mCpsmCpsmGmUfGmUfGfCfAmCmUmUmCmGmCmUmUmCmA-p-(ps)2- |
| | GalNAc4 |
| | (SEQ ID NO: 89) |
| | d2vd3UpsfGpsmAmAfGmCmGfAmAmGmUmGmCfAmCmAfCmGmGpsaUps |
| | mC |
| | (SEQ ID NO: 90) |
| ds-siNA-O33 | mCpsmCpsmGmUfGmUfGfCfAmCmUmUmCmGmCmUmUmCmA-p-(ps)2- |
| | GalNAc4 |
| | (SEQ ID NO: 91) |
| | aUpsfGpsmAmAfGmCmGfAmAmGmUmGmCfAmCmAfCmGmGpsmUpsmC |
| | (SEQ ID NO: 92) |

mX = 2′-O-methyl nucleotide;
fX = 2′-fluoro nucleotide;
5dcd3X = nucleotide of Formula 17;
5dfX = nucleotide of Formula 16;
vX= 5′ vinyl phosphonate nucleotide;
d2vX = deuterated 5′ vinyl phosphonate nucleotide;
vmX = 5′ vinyl phosphonate, 2′-O-methyl nucleotide;
d2vd3U = 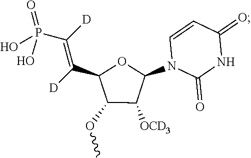
aU = 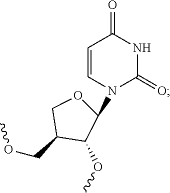
ps = phosphorothioate linkage;
X is a nucleobase (e.g. A, G, C, U or T)

### Target Gene

Without wishing to be bound by theory, upon entry into a cell, any of the ds-siNA molecules disclosed herein may interact with proteins in the cell to form a RNA-Induced Silencing Complex (RISC). Once the ds-siNA is part of the RISC, the ds-siNA may be unwound to form a single-stranded siNA (ss-siNA). The ss-siNA may comprise the antisense strand of the ds-siNA. The antisense strand may bind to a complementary messenger RNA (mRNA), which results in silencing of the gene that encodes the mRNA.

The target gene may be any gene in a cell. In some embodiments, the target gene is a viral gene. In some embodiments, the viral gene is from a DNA virus. In some embodiments, the DNA virus is a double-stranded DNA (dsDNA) virus. In some embodiments, the dsDNA virus is a hepadnavirus. In some embodiments, the hepadnavirus is a hepatitis B virus (HBV). In some embodiments, the HBV is selected from HBV genotypes A-J. In some embodiments, the viral disease is caused by an RNA virus. In some embodiments, the RNA virus is a single-stranded RNA virus (ssRNA virus). In some embodiments, the ssRNA virus is a positive-sense single-stranded RNA virus ((+)ssRNA virus). In some embodiments, the (+)ssRNA virus is a coronavirus. In some embodiments, the coronavirus is a P-coronaviruses. In some embodiments, the P-coronaviruses is selected from the group consisting of severe acute respiratory syndrome coronavirus 2 (SARS-CoV-2) (also known by the provisional name 2019 novel coronavirus, or 2019-nCoV), human coronavirus OC43 (hCoV-OC43), Middle East respiratory syndrome-related coronavirus (MERS-CoV, also known by the provisional name 2012 novel coronavirus, or 2012-nCoV), and severe acute respiratory syndrome-related coronavirus (SARS-CoV, also known as SARS-CoV-1). In some embodiments, the 0-coronaviruses is SARS-CoV-2, the causative agent of COVID-19.Some exemplary target genes are shown in Table 17 at the end of the specification.

In some embodiments, the target gene is selected from the S gene or X gene of the HBV. In some embodiments, the HBV has a genome sequence shown in the nucleotide sequence of SEQ ID NO: 55 which corresponds to the nucleotide sequence of GenBank Accession No. U95551.1, which is incorporated by reference in its entirety.

An exemplary HBV genome sequence is shown in SEQ ID NO: 60, corresponding to Genbank Accession No. KC315400.1, which is incorporated by reference in its entirety. Nucleotides 2307 . . . 3215, 1 . . . 1623 of SEQ ID NO: 60 correspond to the polymerase/RT gene sequence, which encodes for the polymerase protein. Nucleotides 2848 . . . 3215, 1 . . . 835 of SEQ ID NO: 60 correspond to the PreS1/S2/S gene sequence, which encodes for the large S protein. Nucleotides 3205 . . . 3215, 1 . . . 835 of SEQ ID NO: 60 correspond to the PreS2/S gene sequence, which encodes for the middle S protein. Nucleotides 155 . . . 835 of SEQ ID NO: 60 correspond to the S gene sequence, which encodes the small S protein. Nucleotides 1374 . . . 1838 of SEQ ID NO: 60 correspond to the X gene sequence, which encodes the X protein. Nucleotides 1814 . . . 2452 of SEQ ID NO: 60 correspond to the PreC/C gene sequence, which encodes the precore/core protein. Nucleotides 1901 . . . 2452 of SEQ ID NO: 60 correspond to the C gene sequence, which encodes the core protein. The HBV genome further comprises viral regulatory elements, such as viral promoters (preS2, preS1, Core, and X) and enhancer elements (ENH1 and ENH2). Nucleotides 1624 . . . 1771 of SEQ ID NO: 60 correspond to ENH2. Nucleotides 1742 . . . 1849 of SEQ ID NO: 60 correspond to the Core promoter. Nucleotides 1818.3215, 1.1930 of SEQ ID NO: 60 correspond to the pregenomic RNA (pgRNA), which encodes the core and polymerase proteins.

In some embodiments, the sense strand comprises a sequence that is at least about 60%, 65%, 70%, 75%, 80%, 85%, 90%, 95%, or 100% complementary or hybridizes to a viral target RNA sequence that begins in an X region of HBV or in an S region of HBV. The viral target may, e.g., begin at the 5′-end of target-site in acc. KC315400.1 (genotype B, “gt B”), or in any one of genotypes A, C, or D. The skilled person would understand the HBV position, e.g., as described in Wing-Kin Sung, et al., Nature Genetics 44:765 (2012). In some embodiments, the S region is defined as from the beginning of small S protein (in genotype B KC315400.1 isolate, position #155) to before beginning of X protein (in genotype B KC315400.1 isolate, position #1373). In some embodiments, the X region is defined as from the beginning X protein (in genotype B KC315400.1 isolate, position #1374) to end of DR2 site (in genotype B KC315400.1 isolate, position #1603).

In some embodiments, the second nucleotide sequence is at least about 60%, 65%, 70%, 75%, 80%, 85%, 90%, 95%, or 100% complementary to 15 to 30, 15 to 25, 15 to 23, 15 to 22, 15 to 21, 17 to 25, 17 to 23, 17 to 22, 17 to 21, or 19 to 21 nucleotides within positions 200-720 or 1100-1700 of SEQ ID NO: 55. In some embodiments, the second nucleotide sequence is at least about 60%, 65%, 70%, 75%, 80%, 85%, 90%, 95%, or 100% complementary to 15 to 30, 15 to 25, 15 to 23, 15 to 22, 15 to 21, 17 to 25, 17 to 23, 17 to 22, 17 to 21, or 19 to 21 nucleotides within positions 200-280, 300-445, 460-510, 650-720, 1170-1220, 1250-1300, or 1550-1630 of SEQ ID NO: 55. In some embodiments, the second nucleotide sequence is at least about 60%, 65%, 70%, 75%, 80%, 85%, 90%, 95%, or 100% complementary to 15 to 30, 15 to 25, 15 to 23, 15 to 22, 15 to 21, 17 to 25, 17 to 23, 17 to 22, 17 to 21, or 19 to 21 nucleotides within positions 200-230, 250-280, 300-330, 370-400, 405-445, 460-500, 670-700, 1180-1210, 1260-1295, 1520-1550, or 1570-1610 of SEQ ID NO: 55. In some embodiments, the second nucleotide sequence is at least about 60%, 65%, 70%, 75%, 80%, 85%, 90%, 95%, or 100% complementary to 15 to 30, 15 to 25, 15 to 23, 15 to 22, 15 to 21, 17 to 25, 17 to 23, 17 to 22, 17 to 21, or 19 to 21 nucleotides starting at position 203, 206, 254, 305, 375, 409, 412, 415, 416, 419, 462, 466, 467, 674, 676, 1182, 1262, 1263, 1268, 1526, 1577, 1578, 1580, 1581, 1583, or 1584 of SEQ ID NO: 55.

In some embodiments, the first nucleotide is at least about 60%, 65%, 70%, 75%, 80%, 85%, 90%, 95%, or 100% identical to a nucleotide region within SEQ ID NO: 55, with the exception that the thymines (Ts) in SEQ ID NO: 55 are replaced with uracil (U). In some embodiments, the first nucleotide sequence is at least about 60%, 65%, 70%, 75%, 80%, 85%, 90%, 95%, or 100% identical to 15 to 30, 15 to 25, 15 to 23, 15 to 22, 15 to 21, 17 to 25, 17 to 23, 17 to 22, 17 to 21, or 19 to 21 nucleotides within positions 200-720 or 1100-1700 of SEQ ID NO: 55. In some embodiments, the first nucleotide sequence is at least about 60%, 65%, 70%, 75%, 80%, 85%, 90%, 95%, or 100% identical to 15 to 30, 15 to 25, 15 to 23, 15 to 22, 15 to 21, 17 to 25, 17 to 23, 17 to 22, 17 to 21, or 19 to 21 nucleotides within positions 200-280, 300-445, 460-510, 650-720, 1170-1220, 1250-1300, or 1550-1630 of SEQ ID NO: 55. In some embodiments, the first nucleotide sequence is at least about 60%, 65%, 70%, 75%, 80%, 85%, 90%, 95%, or 100% identical to 15 to 30, 15 to 25, 15 to 23, 15 to 22, 15 to 21, 17 to 25, 17 to 23, 17 to 22, 17 to 21, or 19 to 21 nucleotides within positions 200-230, 250-280, 300-330, 370-400, 405-445, 460-500, 670-700, 1180-1210, 1260-1295, 1520-1550, or 1570-1610 of SEQ ID NO: 55. In some embodiments, the first nucleotide sequence is at least about 60%, 65%, 70%, 75%, 80%, 85%, 90%, 95%, or 100% identical to 15 to 30, 15 to 25, 15 to 23, 15 to 22, 15 to 21, 17 to 25, 17 to 23, 17 to 22, 17 to 21, or 19 to 21 nucleotides starting at position 203, 206, 254, 305, 375, 409, 412, 415, 416, 419, 462, 466, 467, 674, 676, 1182, 1262, 1263, 1268, 1526, 1577, 1578, 1580, 1581, 1583, or 1584 of SEQ ID NO: 55.

Several disease-causing coronaviruses share a high degree of homology in the regions of the genome encoding non-structural proteins (nsp), and more specifically, in the region encoding nsp8-nsp15. Indeed, there is roughly 65% identity across the roughly 7 kB sequence of P-coronaviruses from about nucleotide 12900 to about nucleotide 19900 of 2019-nCoV, and some subsections of the genomic span of nsp8 to nsp15 may comprise 95% or more identity. All of the genes in this region encode non-structural proteins associated with replication. Accordingly, this segment of the genome is suitable for targeting with an siNA that can provide a broad spectrum treatment for multiple different types of coronavirus, such as MERS-CoV, SARS-CoV-1, and SARS-CoV-2.

In some embodiments, the target gene is selected from genome of SARS-CoV-2. In some embodiments, SARS-CoV-2 has a genome sequence shown in the nucleotide sequence of SEQ ID NO: 113, which corresponds to the nucleotide sequence of GenBank Accession No. NC_045512.2, which is incorporated by reference in its entirety. In some embodiments, the target gene a sequence 15 to 30, 15 to 25, 15 to 23, 17 to 23, 19 to 23, or 19 to 21 nucleotides in length, and preferably 19 or 21 nucleotides in length, within SEQ ID NO: 113. In some embodiments, the antisense strand sequence is complementary to 15 to 30, 15 to 25, 15 to 23, 15 to 22, 15 to 21, 17 to 25, 17 to 23, 17 to 22, 17 to 21, or 19 to 21 nucleotides, and preferably 19 to 21 nucleotides, and more preferably 19 or 21 nucleotides, within positions 190-216, 233-279, 288-324, 455-477, 626-651, 704-723, 3352-3378, 5384-5403, 6406-6483, 7532-7551, 9588-9606, 10484-10509, 11609-11630, 11834-11853, 12023-12045, 12212-12234, 12401-12420, 12839-12867, 12885-12924, 12966-12990, 13151-13176, 13363-13386, 13388-13416, 13458-13416, 13458-13520, 13762-13790, 14290-14312, 14404-14429, 14500-14531, 14623-14642, 14650-14687, 14698-14717, 14722-14748, 14750-14777, 14821-14846, 14854-14873, 14875-14903, 14962-14990, 14992-15020, 15055-15140, 15172-15200, 15310-15332, 15346-15367, 15496-15518, 15622-15644, 15838-15869, 15886-15905, 15985-16010, 16057-16079, 16186-16205, 16430-16448, 16822-16865, 16954-16976, 17008-17042, 17080-17111, 17137-17156, 17269-17289, 17530-17549, 17563-17582, 17680-17699, 17746-17765, 17857-17876, 17956-17975, 18100-18122, 18196-18218, 19618-19639, 19783-19802, 19831-19850, 20107-20130, 20776-20795, 21502-21524, 24302-24325, 24446-24465, 24620-24651, 24662-24684, 25034-25057, 25104-25128, 25364-25387, 25502-25530, 26191-26227, 26232-26267, 26269-26330, 26332-26394, 26450-26481, 26574-26600, 27003-27064, 27093-27111, 27183-27212, 27382-27407, 27511-27533, 27771-27818, 28270-28296, 28397-28434, 28513-28546, 28673-28692, 28706-28726, 28744-28794, 28799-28827, 28946-28972, 28976-29034, 29144-29172, 29174-29196, 29228-29259, 29285-29305, 29342-29394, 29444-29463, 29543-29566, 29598-29630, 29652-29687, 29689-29731, 29733-29757, or 29770-29828 of SEQ ID NO: 113. In some embodiments, the sense strand sequence is identical to 15 to 30, 15 to 25, 15 to 23, 15 to 22, 15 to 21, 17 to 25, 17 to 23, 17 to 22, 17 to 21, or 19 to 21 nucleotides, and preferably 19 to 21 nucleotides, and more preferably 19 or 21 nucleotides, within positions 190-216, 233-279, 288-324, 455-477, 626-651, 704-723, 3352-3378, 5384-5403, 6406-6483, 7532-7551, 9588-9606, 10484-10509, 11609-11630, 11834-11853, 12023-12045, 12212-12234, 12401-12420, 12839-12867, 12885-12924, 12966-12990, 13151-13176, 13363-13386, 13388-13416, 13458-13416, 13458-13520, 13762-13790, 14290-14312, 14404-14429, 14500-14531, 14623-14642, 14650-14687, 14698-14717, 14722-14748, 14750-14777, 14821-14846, 14854-14873, 14875-14903, 14962-14990, 14992-15020, 15055-15140, 15172-15200, 15310-15332, 15346-15367, 15496-15518, 15622-15644, 15838-15869, 15886-15905, 15985-16010, 16057-16079, 16186-16205, 16430-16448, 16822-16865, 16954-16976, 17008-17042, 17080-17111, 17137-17156, 17269-17289, 17530-17549, 17563-17582, 17680-17699, 17746-17765, 17857-17876, 17956-17975, 18100-18122, 18196-18218, 19618-19639, 19783-19802, 19831-19850, 20107-20130, 20776-20795, 21502-21524, 24302-24325, 24446-24465, 24620-24651, 24662-24684, 25034-25057, 25104-25128, 25364-25387, 25502-25530, 26191-26227, 26232-26267, 26269-26330, 26332-26394, 26450-26481, 26574-26600, 27003-27064, 27093-27111, 27183-27212, 27382-27407, 27511-27533, 27771-27818,28270-28296, 28397-28434,28513-28546, 28673-28692, 28706-28726, 28744-28794, 28799-28827, 28946-28972, 28976-29034, 29144-29172, 29174-29196, 29228-29259, 29285-29305, 29342-29394, 29444-29463, 29543-29566, 29598-29630, 29652-29687, 29689-29731, 29733-29757, or 29770-29828 of SEQ ID NO: 113.

In some embodiments, the target gene is selected from genome of SARS-CoV. In some embodiments, SARS-CoV has a genome corresponding to the nucleotide sequence of GenBank Accession No. NC_004718.3, which is incorporated by reference in its entirety.

In some embodiments, the target gene is selected from the genome of MERS-CoV. In some embodiments, MERS-CoV has a genome corresponding to the nucleotide sequence of GenBank Accession No. NC_019843.3, which is incorporated by reference in its entirety.

In some embodiments, the target gene is selected from the genome of hCoV-OC43. In some embodiments, hCoV-OC43 has a genome corresponding to the nucleotide sequence of GenBank Accession No. NC_006213.1, which is incorporated by reference in its entirety.

In some embodiments, the target gene is involved in liver metabolism. In some embodiments, the target gene is an inhibitor of the electron transport chain. In some embodiments, the target gene encodes the MCJ protein (MCJ/DnaJC15 or Methylation-Controlled J protein). In some embodiments, the MCJ protein is encoded by the mRNA sequence of SEQ ID NO: 56, which corresponds to the nucleotide sequence of GenBank Accession No. NM_013238.3, which is incorporated by reference in its entirety.

In some embodiments, the target gene is TAZ. In some embodiments, TAZ comprises the nucleotide sequence of SEQ ID NO: 57, which corresponds to the nucleotide sequence of GenBank Accession No. NM_000116.5, which is incorporated by reference in its entirety.

In some embodiments, the target gene is angiopoietin like 3 (ANGPTL3). In some embodiments, ANGPTL3 comprises the nucleotide sequence of SEQ ID NO: 60, which corresponds to the nucleotide sequence of GenBank Accession No. NM_014495.4, which is incorporated by reference in its entirety. In some embodiments, the target gene is diacylglycerol acyltransferase 2 (DGAT2). In some embodiments, DGAT2 comprises the nucleotide sequence of SEQ ID NO: 59, which corresponds to the nucleotide sequence of GenBank Accession No. NM_001253891.1, which is incorporated by reference in its entirety.

### Compositions

As indicated above, the present disclosure provides compositions comprising any of the siNA molecules, sense strands, antisense strands, first nucleotide sequences, or second nucleotide sequences described herein. The compositions may comprise 1, 2, 3, 4, 5, 6, 7, 8, 9, 10, 11, 12 or more siNA molecules described herein. The compositions may comprise a first nucleotide sequence comprising a nucleotide sequence of any one SEQ ID NOs: 1 and 2. In some embodiments, the composition comprises a second nucleotide sequence comprising a nucleotide sequence of any one of SEQ ID NOs: 51-74. In some embodiments, the composition comprises a sense strand comprising a nucleotide sequence of any one of SEQ ID NOs: 1 and 2. In some embodiments, the composition comprises an antisense strand comprising a nucleotide sequence of any one of SEQ ID NOs: 51-74.

Alternatively, the compositions may comprise (a) a phosphorylation blocker; and (b) a short interfering nucleic acid (siNA). In some embodiments, the phosphorylation blocker is any of the phosphorylation blockers disclosed herein. In some embodiments, the siNA is any of the siNAs disclosed herein. In some embodiments, the siNA comprises any of the sense strands, antisense strands, first nucleotide sequences, or second nucleotide sequences described herein. In some embodiments, the siNA comprises any of the sense strands, antisense strands, first nucleotide sequences, or second nucleotide sequences described herein. In some embodiments, the siNA comprises one or more modified nucleotides. In some embodiments, the one or more modified nucleotides are independently selected from a 2′-fluoro nucleotide and a 2′-O-methyl nucleotide. In some embodiments, the 2′-fluoro nucleotide or the 2′-O-methyl nucleotide is independently selected from any of the 2′-fluoro or 2′-O-methyl nucleotide mimics disclosed herein. In some embodiments, the siNA comprises a nucleotide sequence comprising any of the modification patterns disclosed herein.

In some embodiments, the composition comprises (a) a conjugated moiety; and (b) a short interfering nucleic acid (siNA). In some embodiments, the conjugated moiety is any of the galactosamines disclosed herein. In some embodiments, the siNA is any of the siNAs disclosed herein. In some embodiments, the siNA comprises any of the sense strands, antisense strands, first nucleotide sequences, or second nucleotide sequences described herein. In some embodiments, the siNA comprises any of the sense strands, antisense strands, first nucleotide sequences, or second nucleotide sequences described herein. In some embodiments, the siNA comprises one or more modified nucleotides. In some embodiments, the one or more modified nucleotides are independently selected from a 2′-fluoro nucleotide and a 2′-O-methyl nucleotide. In some embodiments, the 2′-fluoro nucleotide or the 2′-O-methyl nucleotide is independently selected from any of the 2′-fluoro or 2′-O-methyl nucleotide mimics disclosed herein. In some embodiments, the siNA comprises a nucleotide sequence comprising any of the modification patterns disclosed herein.

In some embodiments, the composition comprises (a) a 5′-stabilized end cap; and (b) a short interfering nucleic acid (siNA). In some embodiments, the 5′-stabilized end cap is any of the 5-stabilized end caps disclosed herein. In some embodiments, the siNA is any of the siNAs disclosed herein. In some embodiments, the siNA comprises any of the sense strands, antisense strands, first nucleotide sequences, or second nucleotide sequences described herein. In some embodiments, the siNA comprises one or more modified nucleotides. In some embodiments, the one or more modified nucleotides are independently selected from a 2′-fluoro nucleotide and a 2′-O-methyl nucleotide. In some embodiments, the 2′-fluoro nucleotide or the 2′-O-methyl nucleotide is independently selected from any of the 2′-fluoro or 2′-O-methyl nucleotide mimics disclosed herein. In some embodiments, the siNA comprises a nucleotide sequence comprising any of the modification patterns disclosed herein.

In some embodiments, the composition comprises (a) at least one phosphorylation blocker, conjugated moiety, or 5′-stabilized end cap; and (b) a short interfering nucleic acid (siNA). In some embodiments, the phosphorylation blocker is any of the phosphorylation blockers disclosed herein. In some embodiments, the conjugated moiety is any of the galactosamines disclosed herein. In some embodiments, the 5′-stabilized end cap is any of the 5-stabilized end caps disclosed herein. In some embodiments, the siNA is any of the siNAs disclosed herein. In some embodiments, the siNA comprises any of the sense strands, antisense strands, first nucleotide sequences, or second nucleotide sequences described herein. In some embodiments, the siNA comprises one or more modified nucleotides. In some embodiments, the one or more modified nucleotides are independently selected from a 2′-fluoro nucleotide and a 2′-O-methyl nucleotide. In some embodiments, the 2′-fluoro nucleotide or the 2′-O-methyl nucleotide is independently selected from any of the 2′-fluoro or 2′-O-methyl nucleotide mimics disclosed herein. In some embodiments, the siNA comprises a nucleotide sequence comprising any of the modification patterns disclosed herein.

The composition may be a pharmaceutical composition. In some embodiments, the pharmaceutical composition comprises an amount of one or more of the siNA molecules described herein formulated with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “therapeutically-effective amount” as used herein means that amount of a compound, material, or composition comprising a siNA of the present disclosure which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present disclosure include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound (e.g., siNA molecule) which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present disclosure comprises an excipient selected from the group consisting of cyclodextrins, celluloses, liposomes, micelle forming agents, e.g., bile acids, and polymeric carriers, e.g., polyesters and polyanhydrides; and a compound (e.g., siNA molecule) of the present disclosure. In certain embodiments, an aforementioned formulation renders orally bioavailable a compound (e.g., siNA molecule) of the present disclosure.

Methods of preparing these formulations or compositions include the step of bringing into association a compound (e.g., siNA molecule) of the present disclosure with the carrier and, optionally, one or more accessory ingredients. In general, the formulations are prepared by uniformly and intimately bringing into association a compound (e.g., siNA molecule) of the present disclosure with liquid carriers, or finely divided solid carriers, or both, and then, if necessary, shaping the product.

Formulations of the disclosure suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound (e.g., siNA molecule) of the present disclosure as an active ingredient. A compound (e.g., siNA molecule) of the present disclosure may also be administered as a bolus, electuary or paste.

In solid dosage forms of the disclosure for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose.

In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried.

They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds (e.g., siNA molecules) of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (I particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds (e.g., siNA molecules), may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the disclosure for rectal or vaginal administration may be presented as a suppository, which may be prepared by mixing one or more compounds (e.g., siNA molecules) of the disclosure with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound (e.g., siNA molecule).

Formulations of the present disclosure which are suitable for vaginal administration also include pessaries, tampons, creams, gels, pastes, foams or spray formulations containing such carriers as are known in the art to be appropriate.

Dosage forms for the topical or transdermal administration of a compound (e.g., siNA molecule) of this disclosure include powders, sprays, ointments, pastes, creams, lotions, gels, solutions, patches and inhalants. The active compound (e.g., siNA molecule) may be mixed under sterile conditions with a pharmaceutically acceptable carrier, and with any preservatives, buffers, or propellants which may be required.

The ointments, pastes, creams and gels may contain, in addition to an active compound (e.g., siNA molecule) of this disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound (e.g., siNA molecule) of this disclosure, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound (e.g., siNA molecule) of the present disclosure to the body. Such dosage forms can be made by dissolving or dispersing the compound (e.g., siNA molecule) in the proper medium. Absorption enhancers can also be used to increase the flux of the compound (e.g., siNA molecule) across the skin. The rate of such flux can be controlled by either providing a rate controlling membrane or dispersing the compound (e.g., siNA molecule) in a polymer matrix or gel.

Ophthalmic formulations, eye ointments, powders, solutions and the like, are also contemplated as being within the scope of this disclosure.

Pharmaceutical compositions of this disclosure suitable for parenteral administration comprise one or more compounds (e.g., siNA molecules) of the disclosure in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds (e.g., siNA molecules) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds (e.g., siNA molecules) of the present disclosure are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

### Treatments and Administration

The siNA molecules of the present disclosure may be used to treat a disease in a subject in need thereof. In some embodiments, a method of treating a disease in a subject in need thereof comprises administering to the subject any of the siNA molecules disclosed herein. In some embodiments, a method of treating a disease in a subject in need thereof comprises administering to the subject any of the compositions disclosed herein.

The preparations (e.g., siNA molecules or compositions) of the present disclosure may be given orally, parenterally, topically, or rectally. They are of course given in forms suitable for each administration route. For example, they are administered in tablets or capsule form, administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds (e.g., siNA molecules) of the present disclosure, which may be used in a suitable hydrated form, and/or the pharmaceutical compositions of the present disclosure, are formulated into pharmaceutically-acceptable dosage forms by conventional methods known to those of skill in the art.

Actual dosage levels of the active ingredients in the pharmaceutical compositions of this disclosure may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound (e.g., siNA molecule) of the present disclosure employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds (e.g., siNA molecules) of the disclosure employed in the pharmaceutical composition at levels lower than that required in order to achieve the desired therapeutic effect and gradually increase the dosage until the desired effect is achieved.

In general, a suitable daily dose of a compound (e.g., siNA molecule) of the disclosure is the amount of the compound that is the lowest dose effective to produce a therapeutic effect. Such an effective dose generally depends upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. In some embodiments, the compound is administered at a dose equal to or greater than 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, 0.50, 0.55, 0.60, 0.65, 0.7, 0.75, 0.8, 0.85, 0.9, 0.95, or 1 mg/kg. In some embodiments, the compound is administered at a dose equal to or less than 200, 190, 180, 170, 160, 150, 140, 130, 120, 110, 100, 95, 90, 85, 80, 75, 70, 65, 60, 55, 50, 45, 40, 35, 30, 25, 20, or 15 mg/kg. In some embodiments, the total daily dose of the compound is equal to or greater than 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 100 mg.

When the compounds (e.g., siNA molecules) described herein are co-administered with another, the effective amount may be less than when the compound is used alone.

If desired, the effective daily dose of the active compound (e.g., siNA molecule) may be administered as two, three, four, five, six or more sub-doses administered separately at appropriate intervals throughout the day, optionally, in unit dosage forms. Preferred dosing is one administration per day. In some embodiments, the compound is administered at least 1, 2, 3, 4, 5, 6, 7, 8, 9, 10, 11, 12, 13, 14, 15, 16, 17, 18, 19, 20, or 21 times a week. In some embodiments, the compound is administered at least 1, 2, 3, 4, 5, 6, 7, 8, 9, 10, 11, 12, 13, 14, 15, 16, 17, 18, 19, 20, or 21 times a month. In some embodiments, the compound is administered once every 1, 2, 3, 4, 5, 6, 7, 8, 9, 10, 11, 12, 13, 14, 15, 16, 17, 18, 19, 20, or 21 days. In some embodiments, the compound is administered once every 1, 2, 3, 4, 5, 6, 7, or 8 weeks.

### Diseases

The siNA molecules and compositions described herein may be administered to a subject to treat a disease. Further disclosed herein are uses of any of the siNA molecules or compositions disclosed herein in the manufacture of a medicament for treating a disease.

In some embodiments, the disease is a viral disease. In some embodiments, the viral disease is caused by a DNA virus. In some embodiments, the DNA virus is a double stranded DNA (dsDNA virus). In some embodiments, the dsDNA virus is a hepadnavirus. In some embodiments, the hepadnavirus is a hepatitis B virus (HBV).

In some embodiments, the disease is a liver disease. In some embodiments, the liver disease is nonalcoholic fatty liver disease (NAFLD). In some embodiments, the NAFLD is nonalcoholic steatohepatitis (NASH). In some embodiments, the liver disease is hepatocellular carcinoma (HCC).

The siNA molecules of the present disclosure may be used to treat or prevent a disease in a subject in need thereof. In some embodiments, a method of treating or preventing a disease in a subject in need thereof comprises administering to the subject any of the siNA molecules disclosed herein. In some embodiments, a method of treating or preventing a disease in a subject in need thereof comprises administering to the subject any of the compositions disclosed herein.

In some embodiments of the disclosed methods and uses, the disease is a respiratory disease. In some embodiments, the respiratory disease is a viral infection. In some embodiments, the respiratory disease is viral pneumonia. In some embodiments, the respiratory disease is an acute respiratory infection. In some embodiments, the respiratory disease is a cold. In some embodiments, the respiratory disease is severe acute respiratory syndrome (SARS). In some embodiments, the respiratory disease is Middle East respiratory syndrome (MERS). In some embodiments, the disease is coronavirus disease 2019 (e.g., COVID-19). In some embodiments, the respiratory disease can include one or more symptoms selected from coughing, sore throat, runny nose, sneezing, headache, fever, shortness of breath, myalgia, abdominal pain, fatigue, difficulty breathing, persistent chest pain or pressure, difficulty waking, loss of smell and taste, muscle or joint pain, chills, nausea or vomiting, nasal congestion, diarrhea, haemoptysis, conjunctival congestion, sputum production, chest tightness, and palpitations. In some embodiments, the respiratory disease can include complications selected from sinusitis, otitis media, pneumonia, acute respiratory distress syndrome, disseminated intravascular coagulation, pericarditis, and kidney failure. In some embodiments, the respiratory disease is idiopathic.

In some embodiments, the present disclosure provides methods of treating or preventing a coronavirus infection, comprising administering to a subject in need thereof a therapeutically effective amount of one or more of the siNAs or a pharmaceutical composition as disclosed herein. In some embodiments, the coronavirus infection is selected from the group consisting of Middle East Respiratory Syndrome (MERS), Severe Acute Respiratory Syndrome (SARS), and COVID-19. In some embodiments, the subject has been treated with one or more additional coronavirus treatment agents. In some embodiments, the subject is concurrently treated with one or more additional coronavirus treatment agents.

Administration of siNA

Administration of any of the siNAs disclosed herein may be conducted by methods known in the art. In some embodiments, the siNA is administered by subcutaneous (SC) or intravenous (IV) delivery. The preparations (e.g., siNAs or compositions) of the present disclosure may be given orally, parenterally, topically, or rectally. They are of course given in forms suitable for each administration route. For example, they are administered in tablets or capsule form, administration by injection, infusion or inhalation; topical by lotion or ointment; and rectal by suppositories. In some embodiments, subcutaneous administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds (e.g., siNAs) of the present disclosure, which may be used in a suitable hydrated form, and/or the pharmaceutical compositions of the present disclosure, are formulated into pharmaceutically-acceptable dosage forms by conventional methods known to those of skill in the art.

Actual dosage levels of the active ingredients in the pharmaceutical compositions of this disclosure may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound (e.g., siNA) of the present disclosure employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds (e.g., siNAs) of the disclosure employed in the pharmaceutical composition at levels lower than that required in order to achieve the desired therapeutic effect and gradually increase the dosage until the desired effect is achieved.

In general, a suitable daily dose of a compound (e.g., siNA) of the disclosure is the amount of the compound that is the lowest dose effective to produce a therapeutic effect. Such an effective dose generally depends upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. In some embodiments, the compound is administered at about 1 mg/kg to about 40 mg/kg, about 1 mg/kg to about 30 mg/kg, about 1 mg/kg to about 20 mg/kg, about 1 mg/kg to about 15 mg/kg, or 1 mg/kg to about 10 mg/kg. In some embodiments, the compound is administered at a dose equal to or greater than 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, 0.50, 0.55, 0.60, 0.65, 0.7, 0.75, 0.8, 0.85, 0.9, 0.95, or 1 mg/kg. In some embodiments, the compound is administered at a dose equal to or greater than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mg/kg. In some embodiments, the compound is administered at a dose equal to or less than 200, 190, 180, 170, 160, 150, 140, 130, 120, 110, 100, 95, 90, 85, 80, 75, 70, 65, 60, 55, 50, 45, 40, 35, 30, 25, 20, or 15 mg/kg. In some embodiments, the total daily dose of the compound is equal to or greater than 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 100 mg.

If desired, the effective daily dose of the active compound (e.g., siNA) may be administered as two, three, four, five, six, seven, eight, nine, ten or more doses or sub-doses administered separately at appropriate intervals throughout the day, optionally, in unit dosage forms. In some embodiments, the compound is administered at least 1, 2, 3, 4, 5, 6, 7, 8, 9, 10, 11, 12, 13, 14, or 15 times. Preferred dosing is one administration per day. In some embodiments, the compound is administered at least 1, 2, 3, 4, 5, 6, 7, 8, 9, 10, 11, 12, 13, 14, 15, 16, 17, 18, 19, 20, or 21 times a week. In some embodiments, the compound is administered at least 1, 2, 3, 4, 5, 6, 7, 8, 9, 10, 11, 12, 13, 14, 15, 16, 17, 18, 19, 20, or 21 times a month. In some embodiments, the compound is administered once every 1, 2, 3, 4, 5, 6, 7, 8, 9, 10, 11, 12, 13, 14, 15, 16, 17, 18, 19, 20, or 21 days. In some embodiments, the compound is administered every 3 days. In some embodiments, the compound is administered once every 1, 2, 3, 4, 5, 6, 7, 8, 9, 10, 11, 12, 13, 14, or 15 weeks. In some embodiments, the compound is administered every month. In some embodiments, the compound is administered once every 1, 2, 3, 4, 5, 6, 7, 8, 9, 10, 11, 12, 13, 14, or 15 months. In some embodiments, the compound is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 times over a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days. In some embodiments, the compound is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 times over a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 weeks. In some embodiments, the compound is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 times over a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 months. In some embodiments, the compound is administered at least once a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the compound is administered at least once a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months. In some embodiments, the compound is administered at least twice a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the compound is administered at least twice a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months. In some embodiments, the compound is administered at least once every two weeks for a period of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the compound is administered at least once every two weeks for a period of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months. In some embodiments, the compound is administered at least once every four weeks for a period of at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the compound is administered at least once every four weeks for a period of at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months.

In some embodiments, any one of the siNAs or compositions disclosed herein is administered in a particle or viral vector. In some embodiments, the viral vector is a vector of adenovirus, adeno-associated virus (AAV), alphavirus, flavivirus, herpes simplex virus, lentivirus, measles virus, picornavirus, poxvirus, retrovirus, or rhabdovirus. In some embodiments, the viral vector is a recombinant viral vector. In some embodiments, the viral vector is selected from AAVrh.74, AAVrh.10, AAVrh.20, AAV-1, AAV-2, AAV-3, AAV-4, AAV-5, AAV-6, AAV-7, AAV-8, AAV-9, AAV-10, AAV-11, AAV-12 and AAV-13.

The subject of the described methods may be a mammal, and it includes humans and non-human mammals. In some embodiments, the subject is a human, such as an adult human.

Some embodiments include a method for treating an HBV virus in a subject infected with the virus comprising administering a therapeutically effective amount of one or more siNA of the present disclosure or a composition of the present disclosure to the subject in need thereof thereby reducing the viral load of the virus in the subject and/or reducing a level of a virus antigen in the subject. The siNA may be complementary or hybridize to a portion of the target RNA in the virus, e.g., an X region and/or an S region of HBV.

### Combination Therapies

Any of the methods disclosed herein may further comprise administering to the subject an additional HBV treatment agent. Any of the compositions disclosed herein may further comprise an additional HBV treatment agent. In some embodiments, the additional HBV treatment agent is selected from a nucleotide analog, nucleoside analog, a capsid assembly modulator (CAM), a recombinant interferon, an entry inhibitor, a small molecule immunomodulator and oligonucleotide therapy. In some embodiments, the additional HBV treatment agent is selected from HBV STOPS™ ALG-010133, HBV CAM ALG-000184, ASO 1 (SEQ ID NO: 61), ASO 2 (SEQ ID NO: 62) recombinant interferon alpha 2b, IFN-α, PEG-IFN-α-2a, lamivudine, telbivudine, adefovir dipivoxil, clevudine, entecavir, tenofovir alafenamide, tenofovir disoproxil, NVR3-778, BAY41-4109, JNJ-632, JNJ-3989 (ARO-HBV), RG6004, GSK3228836, REP-2139, REP-2165, AB-729, VIR-2218, RG6346 (DCR-HBVS), JNJ-6379, GLS4, ABI-HO731, JNJ-440, NZ-4, RG7907, EDP-514, AB-423, AB-506, ABI-H03733 and ABI-H2158. In some embodiments, the oligonucleotide therapy is selected from Nucleic Acid Polymers or S-Antigen Transport-inhibiting Oligonucleotide Polymers (NAPs or STOPS), siRNA, and ASO. In some embodiments, the oligonucleotide therapy is an additional siNA. In some embodiments, the additional siNA is selected from any of ds-siNA-001 to ds-siNA-025. In some embodiments, the oligonucleotide therapy is an antisense oligonucleotide (ASO). In some embodiments, the ASO is ASO 1 (SEQ ID NO: 61) or ASO 2 (SEQ ID NO: 62). In some embodiments, any of the siNAs disclosed herein are co-administered with STOPS. Exemplary STOPS are described in International Publication No. WO2020/097342 and U.S. Publication No. 2020/0147124, both of which are incorporated by reference in their entirety. In some embodiments, the STOPS is ALG-010133. In some embodiments, any of the siNAs disclosed herein are co-administered with tenofovir. In some embodiments, any of the siNAs disclosed herein are co-administered with a CAM. Exemplary CAMs are described in Berke et al., Antimicrob Agents Chemother, 2017, 61(8):e00560-17, Klumpp, et al., Gastroenterology, 2018, 154(3):652-662.e8, International Application Nos. PCT/US2020/017974, PCT/US2020/026116, and PCT/US2020/028349 and U.S. application Ser. Nos. 16/789,298, 16/837,515, and 16/849,851, each which is incorporated by reference in its entirety. In some embodiments, the CAM is ALG-000184, ALG-001075, ALG-001024, JNJ-632, BAY41-4109, or NVR3-778. In some embodiments, the siNA and the HBV treatment agent are administered simultaneously. In some embodiments, the siNA and the HBV treatment agent are administered concurrently. In some embodiments, the siNA and the HBV treatment agent are administered sequentially. In some embodiments, the siNA is administered prior to administering the HBV treatment agent. In some embodiments, the siNA is administered after administering the HBV treatment agent. In some embodiments, the siNA and the HBV treatment agent are in separate containers. In some embodiments, the siNA and the HBV treatment agent are in the same container.

Any of the methods disclosed herein may further comprise administering to the subject a liver disease treatment agent. Any of the compositions disclosed herein may further comprise a liver disease treatment agent. In some embodiments, the liver disease treatment agent is selected from a peroxisome proliferator-activator receptor (PPAR) agonist, farnesoid X receptor (FXR) agonist, lipid-altering agent, and incretin-based therapy. In some embodiments, the PPAR agonist is selected from a PPARα agonist, dual PPARα/6 agonist, PPARγ agonist, and dual PPARα/γ agonist. In some embodiments, the dual PPARα agonist is a fibrate. In some embodiments, the PPARα/6 agonist is elafibranor. In some embodiments, the PPARγ agonist is a thiazolidinedione (TZD). In some embodiments, TZD is pioglitazone. In some embodiments, the dual PPARα/γ agonist is saroglitazar. In some embodiments, the FXR agonist is obeticholic acis (OCA). In some embodiments, the lipid-altering agent is aramchol. In some embodiments, the incretin-based therapy is a glucagon-like peptide 1 (GLP-1) receptor agonist or dipeptidyl peptidase 4 (DPP-4) inhibitor. In some embodiments, the GLP-1 receptor agonist is exenatide or liraglutide. In some embodiments, the DPP-4 inhibitor is sitagliptin or vildapliptin. In some embodiments, the siNA and the liver disease treatment agent are administered concurrently. In some embodiments, the siNA and the liver disease treatment agent are administered sequentially. In some embodiments, the siNA is administered prior to administering the liver disease treatment agent. In some embodiments, the siNA is administered after administering the liver disease treatment agent. In some embodiments, the siNA and the liver disease treatment agent are in separate containers. In some embodiments, the siNA and the liver disease treatment agent are in the same container.

### Definitions

Unless defined otherwise, all technical and scientific terms used herein have the meaning commonly understood by a person skilled in the art to which this disclosure belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al., (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

The terms “a” and “an” as used herein mean “one or more” and include the plural unless the context is inappropriate.

As used herein, the terms “patient” and “subject” refer to organisms to be treated by the methods of the present disclosure. Such organisms are preferably mammals (e.g., marines, simians, equines, bovines, porcinis, canines, felines, and the like), and more preferably humans.

As used herein, the term “effective amount” refers to the amount of a compound (e.g., a siNA of the present disclosure) sufficient to effect beneficial or desired results. An effective amount can be administered in one or more administrations, applications, or dosages and is not intended to be limited to a particular formulation or administration route.

As used herein, the term “treating” includes any effect, e.g., lessening, reducing, modulating, ameliorating or eliminating, that results in the improvement of the condition, disease, disorder, and the like, or ameliorating a symptom thereof.

As used herein, the terms “alleviate” and “alleviating” refer to reducing the severity of the condition, such as reducing the severity by, for example, at least 10%, 20%, 30%, 40%, 50%, 60%, 70%, 80%, 90%, or 95%.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, for example, Martin, Remington's Pharmaceutical Sciences, 15th Ed., Mack Publ. Co., Easton, PA [1975].

The term “about” as used herein when referring to a measurable value (e.g., weight, time, and dose) is meant to encompass variations, such as ±10%, 5%, +1%, or ±0.1% of the specified value.

As used herein, the term “nucleobase” refers to a nitrogen-containing biological compound that forms a nucleoside. Examples of nucleobases include, but are not limited to, thymine, uracil, adenine, cytosine, guanine, and an analogue or derivative thereof.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

As a general matter, compositions specifying a percentage are by weight unless otherwise specified. Further, if a variable is not accompanied by a definition, then the previous definition of the variable controls.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided may be different from the actual publication dates that may need to be independently confirmed.

### EXAMPLES

### Example 1: siNA Synthesis

This example describes an exemplary method for synthesizing ds-siNAs, such as the siNAs disclosed in Tables 1-5 (as identified by the ds-siNA ID).

The 2′-O-Me phosphoramidite 5′-O-DMT-deoxy Adenosine (NH-Bz), 3′-O-(2-cyanoethyl-N,N-diisopropyl phosphoramidite, 5′-O-DMT-deoxy Guanosine (NH-ibu), 3′-O-(2-cyanoethyl-N,N-diisopropyl phosphoramidite, 5′-O-DMT-deoxy Cytosine (NH-Bz), 3′-O-(2-cyanoethyl-N,N-diisopropyl phosphoramidite, 5′-O-DMT-Uridine 3′-O-(2-cyanoethyl-N,N-diisopropyl phosphoramidite and solid supports were purchased from Chemgenes Corp. MA.

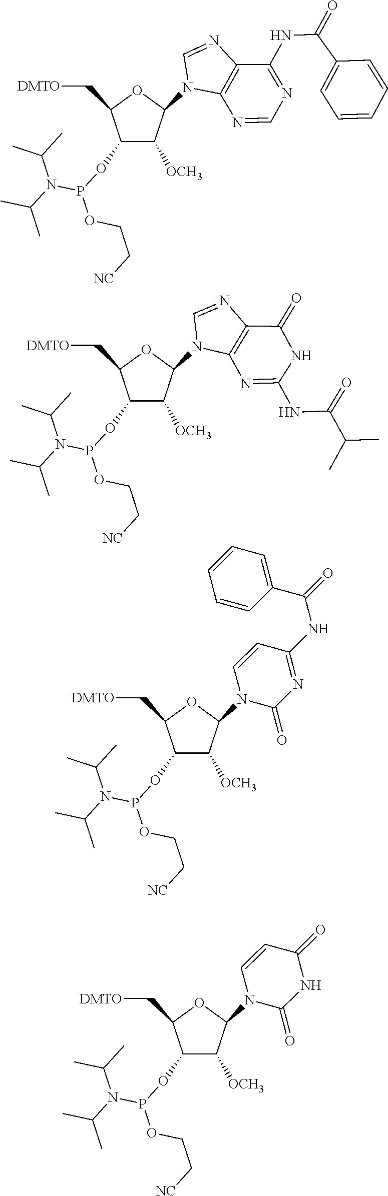

The 2′-F-5′-O-DMT-(NH-Bz) Adenosine-3′-O-(2-cyanoethyl-N,N-diisopropyl phosphoramidite, 2′-F-5′-O-DMT-(NH-ibu)-Guanosine, 3′-O-(2-cyanoethyl-N,N-diisopropyl phosphoramidite, 5′-O-DMT-(NH-Bz)-Cytosine, 2′-F-3′-O-(2-cyanoethyl-N,N-diisopropyl phosphoramidite, 5′-O-DMT-Uridine, 2′-F-3′-O-(2-cyanoethyl-N,N-diisopropyl phosphoramidite and solid supports were purchased from Thermo Fischer Milwaukee WI, USA.

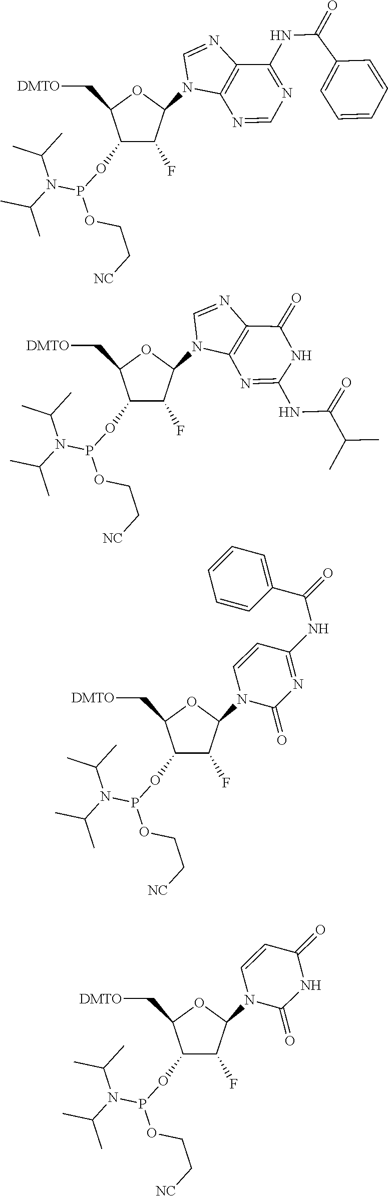

All the monomers were dried in vacuum desiccator with desiccants (P₂O₅, RT 24h). The solid supports (CPG) attached to the nucleosides and universal supports was obtained from LGC and Chemgenes. The chemicals and solvents for post synthesis workflow were purchased from commercially available sources like VWR/Sigma and used without any purification or treatment. Solvent (Acetonitrile) and solutions (amidite and activator) were stored over molecular sieves during synthesis.

The oligonucleotides were synthesized on a DNA/RNA Synthesizers (Expedite 8909 or ABI-394 or MM-48) using standard oligonucleotide phosphoramidite chemistry starting from the 3′ residue of the oligonucleotide preloaded on CPG support. An extended coupling of 0.1M solution of phosphoramidite in CH₃CN in the presence of 5-(ethylthio)-1H-tetrazole activator to a solid bound oligonucleotide followed by standard capping, oxidation and deprotection afforded modified oligonucleotides. The 0.1M 12, THF:Pyridine; Water-7:2:1 was used as oxidizing agent while DDTT ((dimethylamino-methylidene) amino)-3H-1,2,4-dithiazaoline-3-thione was used as the sulfur-transfer agent for the synthesis of oligoribonucleotide phosphorothioates. The stepwise coupling efficiency of all modified phosphoramidites was more than 98%.

| Reagents | Detailed Description |
|---|---|
| Deblock Solution | 3% Dichloroacetic acid (DCA) in |
| | Dichloromethane (DCM) |
| Amidite Concentration | 0.1M in Anhydrous Acetonitrile |
| Activator | 0.25M Ethyl-thio-Tetrazole (ETT) |
| Cap-A solution | Acetic anhydride in pyridine/THF |
| Cap-B Solution | 16% 1-Methylimidazole in THF |
| Oxidizing Solution | 0.02M I₂, THF:pyridine; water-7:2:l |
| Sulfurizing Solution | 0.2M DDTT in pyridine/acetonitrile 1:1 |

### Cleavage and Deprotection:

Deprotection and cleavage from the solid support was achieved with mixture of ammonia methylamine (1:1, AMA) for 15 min at 65° C. When the universal linker was used, the deprotection was left for 90 min at 65° C. or solid supports were heated with aqueous ammonia (28%) solution at 55° C. for 8-16 h to deprotect the base labile protecting groups.

Quantitation of Crude siNA or Raw Analysis

Samples were dissolved in deionized water (1.0 mL) and quantitated as follows: Blanking was first performed with water alone (2 ul) on Thermo Scientific™ Nanodrop UV spectrophotometer or BioTek™ Epoch™ plate reader then Oligo sample reading was obtained at 260 nm. The crude material is dried down and stored at −20° C.

### Crude HPLC/LC-MS Analysis

The 0.1 OD of the crude samples were analyzed for crude HPLC and LC-MS analysis. After Confirming the crude LC-MS data then purification step was performed if needed based on the purity.

### HPLC Purification

The unconjugated and GalNac modified oligonucleotides were purified by anion-exchange HPLC. The buffers were 20 mM sodium phosphate in 10% CH₃CN, pH 8.5 (buffer A) and 20 mM sodium phosphate in 10% CH₃CN, 1.0 M NaBr, pH 8.5 (buffer B). Fractions containing full-length oligonucleotides were pooled.

### Desalting of Purified SiNA

The purified dry siNA was then desalted using Sephadex G-25 M (Amersham Biosciences). The cartridge was conditioned with 10 mL of deionized water thrice. Finally, the purified siNA dissolved thoroughly in 2.5 mL RNAse free water was applied to the cartridge with very slow drop wise elution. The salt free siNA was eluted with 3.5 ml deionized water directly into a screw cap vial. Alternatively, some unconjugated siNA was deslated using Pall AcroPrep™ 3K MWCO desalting plates.

### IEX HPLC and Electrospray LC/MS Analysis

Approximately 0.10 OD of siNA is dissolved in water and then pipetted into HPLC autosampler vials for IEX-HPLC and LC/MS analysis. Analytical HPLC and ES LC-MS confirmed the identity and purity of the compounds.

### Duplex Preparation:

Single strand oligonucleotides (Sense and Antisense strands) were annealed (1:1 by molar equivalents, heat at 90° C. for 2 min followed by gradual cooling at room temperature) to give the duplex ds-siNA. The final compounds were analyzed on size exclusion chromatography (SEC).

### Example 2

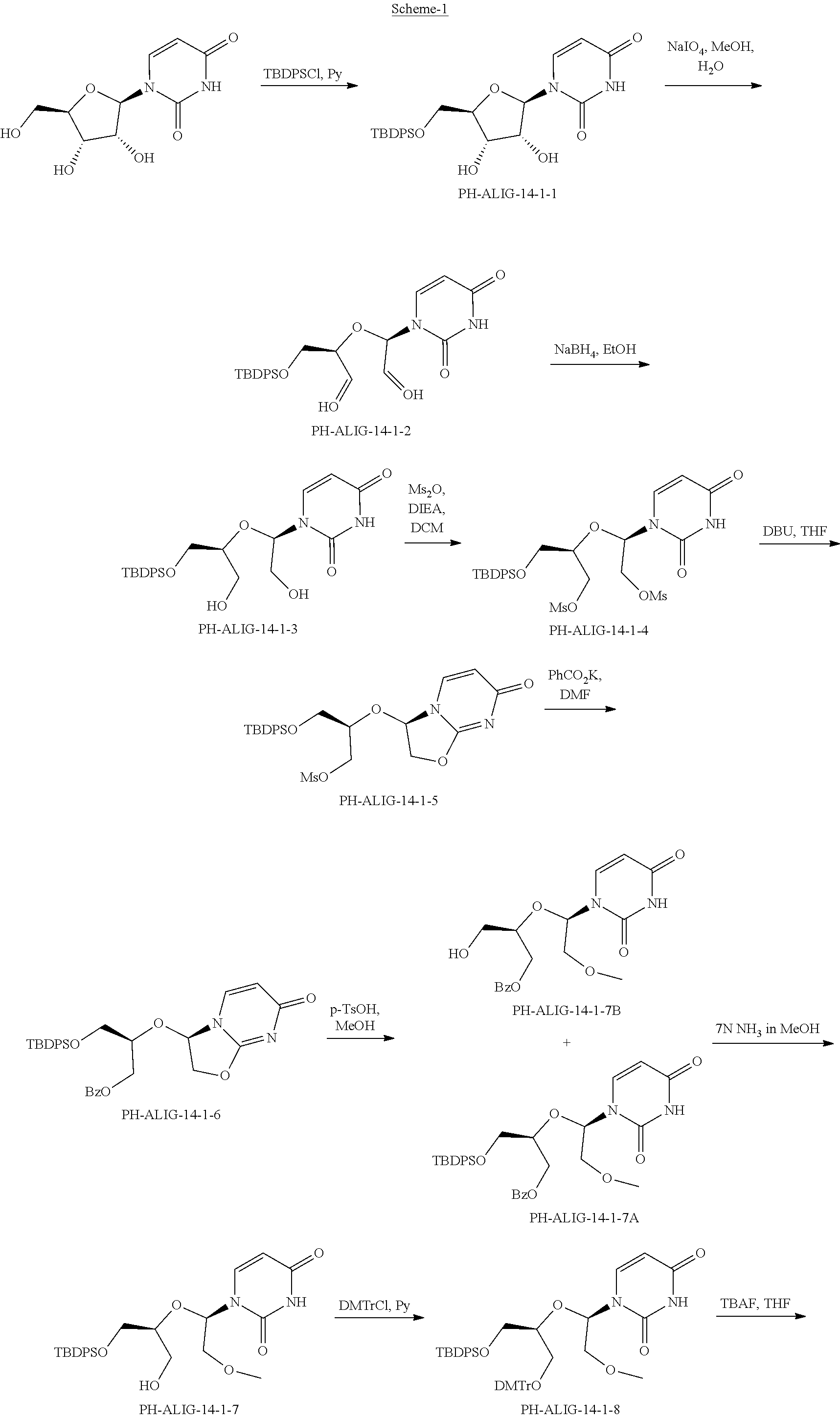

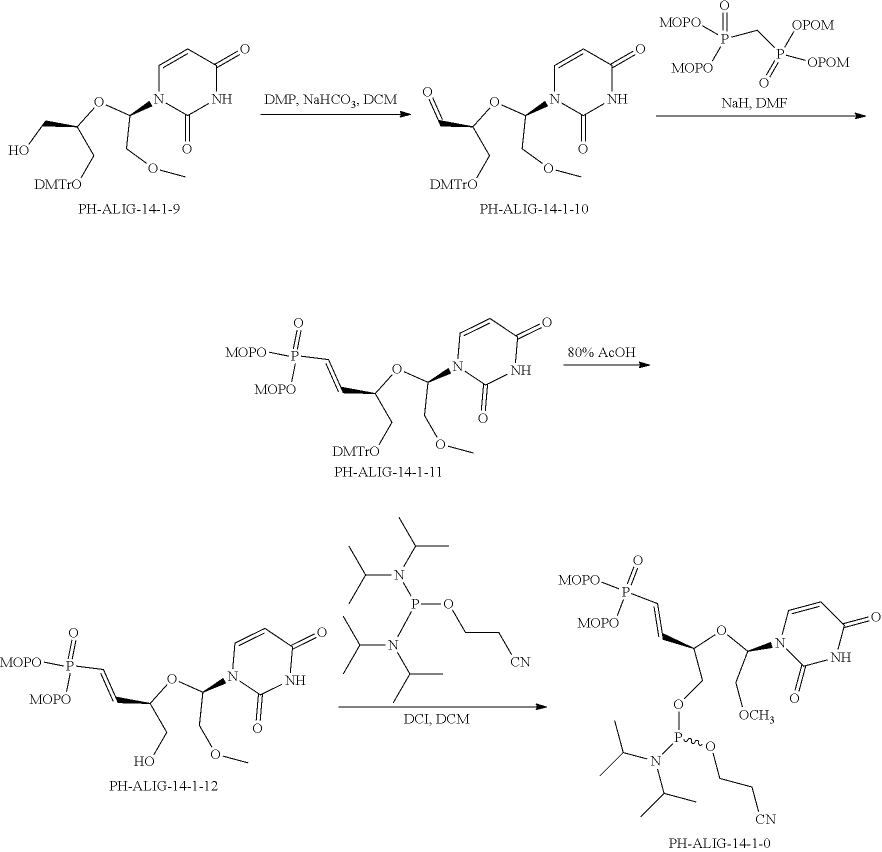

### Preparation of PH-ALIG-14-1-1

Into a 5000-mL 3-necked round-bottom flask purged and maintained with an inert atmosphere of argon, was placed uridine (150.00 g, 614.24 mmol, 1.00 eq), pyridine (2.2 L), TBDPSCl (177.27 g, 644.95 mmol, 1.05 eq). The resulting solution was stirred overnight at room temperature. The resulting mixture was concentrated. The resulting solution was extracted with 3×1000 mL of dichloromethane and the organic layers combined. The resulting mixture was washed with 3×1 L of 0.5N HCl(aq.) and 2×500 mL of 0.5N NaHCO₃ (aq.). The resulting mixture was washed with 2×1 L of H₂O. The mixture was dried over anhydrous sodium sulfate. The solids were filtered out. The filtrate was concentrated. This resulted in 262 g (crude) PH-ALIG-14-1-1. LC-MS (m/z) 483.00 [M+H]⁺; ¹H NMR (400 MHz, DMSO-d6) δ 11.35 (d, J=2.2 Hz, 1H), 7.70 (d, J=8.1 Hz, 1H), 7.64 (m, 4H), 7.52-7.40 (m, 6H), 5.80 (d, J=4.1 Hz, 1H), 5.50 (d, J=5.1 Hz, 1H), 5.28 (dd, J=8.0, 2.2 Hz, 1H), 5.17 (d, J=5.3 Hz, 1H), 4.15-4.05 (m, 2H), 4.00-3.85 (m, 2H), 3.85-3.73 (m, 1H), 1.03 (s, 9H).

### Preparation of PH-ALIG-14-1-2

Into a 10 L 3-necked round-bottom flask purged and maintained with an inert atmosphere of argon, was placed a solution of PH-ALIG-14-1-1 (260.00 g, 538.7 mmol, 1.0 eq.) in MeOH (5000 mL). This was followed by the addition of a solution of NaIO4 (126.8 g, 592.6 mmol, 1.1 eq.) in H₂O (1600 mL) in several batches at 0° C. The resulting solution was stirred for 1 hr at room temperature. The reaction was then quenched by the addition of 3 L of Na₂S₂O₃(sat.) at 0° C. The resulting solution was extracted with 3×1 L of dichloromethane and the organic layers combined and dried over anhydrous sodium sulfate. The solids were filtered out. The filtrate was concentrated. This resulted in 290 g (crude) of PH-ALIG-14-1-2 as a white solid.

### Preparation of PH-ALIG-14-1-3

Into a 5 L 3-necked round-bottom flask purged and maintained with an inert atmosphere of argon, was placed PH-ALIG-14-1-2 (290 g, 603.4 mmol, 1.0 eq), EtOH (3 L). This was followed by the addition of NaBH₄ (22.8 g, 603.4 mmol, 1.0 eq), in portions at 0° C. The resulting solution was stirred for 1 hr at room temperature. The reaction was then quenched by the addition of 2000 mL of water/ice. The resulting solution was extracted with 3×1000 mL of dichloromethane and the organic layers combined and dried over anhydrous sodium sulfate. The solids were filtered out. The filtrate was concentrated. This resulted in 230 g (crude) of PH-ALIG-14-1-3 as a white solid. LC-MS:m/z 485.10 [M+H]⁺. ¹H NMR (400 MHz, DMSO-d6) δ 11.28 (d, J=2.2 Hz, 1H), 7.63-7.37 (m, 11H), 5.84 (dd, J=6.4, 4.9 Hz, 1H), 5.44 (dd, J=8.0, 2.2 Hz, 1H), 5.11 (t, J=6.0 Hz, 1H), 4.78 (t, J=5.2 Hz, 1H), 3.65 (dd, J=11.4, 5.7 Hz, 1H), 3.60-3.52 (m, 5H), 3.18 (d, J=5.2 Hz, 1H), 0.96 (s, 9H).

### Preparation of PH-ALIG-14-1-4

Into a 5000-mL 3-necked round-bottom flask purged and maintained with an inert atmosphere of argon, was placed a solution of PH-ALG-14-1-3 (120 g, 1 eq) in DCM (1200 mL). This was followed by the addition of DIEA (95.03 g, 3 eq) at 0 degrees C. To this was added methanesulfonic anhydride (129 g, 3 eq), in portions at 0° C. The resulting solution was stirred for 1 hr at room temperature. The reaction was then quenched by the addition of 1000 mL of water/ice. The resulting solution was extracted with 3×500 mL of dichloromethane and the organic layers combined and dried over anhydrous magnesium sulfate. The solids were filtered out. The filtrate was concentrated. This resulted in 160 g (crude) of PH-ALG-14-1-4 as a yellow solid.; LC-MS (m/z) 641.05[M+H]+

### Preparation of PH-ALIG-14-1-5

Into a 1 L round-bottom flask, was placed a solution of PH-ALG-14-1-4 (160.00 g, 1.00 equiv) in THE (1600 mL), DBU (108 g, 2.8 equiv). The resulting solution was stirred for 1 hr at 30° C. The reaction was then quenched by the addition of 3000 mL of water/ice. The resulting solution was extracted with 3×500 mL of dichloromethane and the organic layers combined and dried over anhydrous sodium sulfate. The solids were filtered out. The filtrate was concentrated. This resulted in 150 g (crude) of PH-ALG-14-1-5 as brown oil.; LC-MS:(ES, m z):567.25[M+H]+¹HNMR(400 MHz, DMSO-d6) δ 7.83 (d, J=7.4 Hz, 1H), 7.67-7.55 (m, 4H), 7.55-7.35 (m, 6H), 6.05 (dd, J=5.9, 1.7 Hz, 1H), 5.72 (d, J=7.4 Hz, 1H), 4.81 (dd, J=10.4, 5.8 Hz, 1H), 4.58-4.46 (m, 2H), 4.42 (p, J=5.2, 4.6 Hz, 1H), 4.33 (dd, J=10.6, 5.9 Hz, 1H), 3.79-3.70 (m, 2H), 3.23 (s, 3H), 0.98 (s, 9H).

### Preparation of PH-ALIG-14-1-6

Into a 3000-mL round-bottom flask purged and maintained with an inert atmosphere of argon, was placed PH-ALIG-14-1-5 (150.00 g, 201.950 mmol, 1. eq), DMF (1300.00 mL), potassium benzoate (44.00 g, 1.0 eq). The resulting solution was stirred for 1.5 hr at 80° C. The reaction was then quenched by the addition of 500 mL of water/ice. The resulting solution was extracted with 3×500 mL of dichloromethane The resulting mixture was washed with 3×1000 ml of H₂O. The resulting mixture was concentrated. The residue was applied onto a silica gel column with EA/PE (99:1). The collected fractions were combined and concentrated. This resulted in 40 g of PH-ALIG-14-1-6 as yellow oil. LC-MS: m/z 571.20 [M+H]⁺; 1HNMR:(400 MHz, DMSO-d6) δ 7.97-7.91 (m, 2H), 7.89 (d, J=7.4 Hz, 1H), 7.74-7.51 (m, 7H), 7.51-7.31 (m, 6H), 6.16 (m, 1H), 5.76 (d, J=7.4 Hz, 1H), 4.78 (m, 1H), 4.61 (m, 1H), 4.55-4.46 (m, 2H), 4.38 (m, 1H), 3.82 (d, J=5.0 Hz, 2H), 0.97 (s, 9H)

### Preparation of PH-ALIG-14-1-7A

Into a 2-L round-bottom flask, was placed PH-ALIG-14-1-6 (30.00 g, 1 eq), MeOH (1.20 L), p-toluenesulfonic acid (4.50 g, 0.5 eq). The resulting solution was stirred for 2 hr at 70° C. The reaction was then quenched by the addition of 3 L of NaHCO₃ (sat.). The pH value of the solution was adjusted to 7 with NaHCO₃ (sat.). The resulting solution was extracted with 3×1 L of ethyl acetate and the organic layers combined and dried over anhydrous sodium sulfate. The solids were filtered out. The filtrate was concentrated under vacuum. The crude product was purified by Flash-Prep-HPLC with the following conditions (IntelFlash-1): Column, silica gel; mobile phase, PE/EA=50/50 increasing to PE/EA=25/75 within 30; Detector, 254. This resulted in 11.5 g (3.1% yield in seven steps) PH-ALIG-14-1-7A as a white solid. LC-MS: m/z 625.15[M+Na]+; ¹HNMR:(400 MHz, DMSO-d6) δ 11.37 (d, J=2.3 Hz, 1H), 7.99-7.93 (m, 2H), 7.74-7.65 (m, 1H), 7.63-7.50 (m, 7H), 7.50-7.33 (m, 6H), 6.08 (t, J=6.0 Hz, 1H), 5.49 (m, 1H), 4.60 (m, 1H), 4.43 (m, 1H), 4.03-3.96 (m, 1H), 3.70 (d, J=5.3 Hz, 2H), 3.62-3.49 (m, 2H), 3.21 (s, 3H), 0.97 (s, 9H).

### Preparation of PH-ALIG-14-1-7

Into a 2-L round-bottom flask, was placed PH-ALIG-14-1-7A (11.50 g). To the above 7M NH₃(g) in MeOH (690.00 mL) was introduced in at 30° C. The resulting solution was stirred overnight at 30 degrees C. The resulting mixture was concentrated under vacuum. The crude product was purified by Flash with the following conditions (IntelFlash-1): Column, silica gel; mobile phase, PE/EA=60/40 increasing to PE/EA=1/99 within 60; Detector, 254. This resulted in 8.1 g (97% yield) of PH-ALIG-14-1-7 as a white solid. LC-MS-: m/z 499.35 [M+H]⁺; ¹HNMR: (300 MHz, DMSO-d6) δ 11.31 (s, 1H), 7.64-7.50 (m, 5H), 7.48-7.35 (m, 6H), 6.02 (t, J=5.8 Hz, 1H), 5.45 (d, J=8.0 Hz, 1H), 4.80 (t, J=5.1 Hz, 1H), 3.58 (m, 7H), 3.27 (s, 3H), 0.96 (s, 9H).

### Preparation of PH-ALIG-14-1-8

Into a 250-mL round-bottom flask, was placed PH-ALIG-14-1-7(8.10 g, 1 equiv), pyridine (80.0 mL), DMTr-Cl (7.10 g, 1.3eq). The flask was evacuated and flushed three times with Argon. The resulting solution was stirred for 2 hr at room temperature. The reaction was then quenched by the addition of 500 mL of NaHCO₃ (sat.). The resulting solution was extracted with 2×500 mL of ethyl acetate and the organic layers combined and dried over anhydrous sodium sulfate. The solids were filtered out. The filtrate was concentrated under vacuum. The crude product was purified by Flash with the following conditions (IntelFlash-1): Column, C18; mobile phase, ACN/H₂O=5/95 increasing to ACN/H₂O=95/5 within 30; Detector, 254. This resulted in 11.5 g (88% yield) of PH-ALIG-14-1-8 as a white solid.; LC-MS: m/z 823.40 [M+Na]⁺; 1HNMR: (300 MHz, DMSO-d6) δ 11.37 (s, 1H), 7.55-7.18 (m, 20H), 6.92-6.83 (m, 4H), 6.14 (t, J=5.9 Hz, 1H), 5.48 (d, J=8.0 Hz, 1H), 3.74 (m, 7H), 3.57 (m, 4H), 3.25 (m, 5H), 0.84 (s, 9H).

### Preparation of PH-ALIG-14-1-9

Into a 1000-mL round-bottom flask, was placed PH-ALIG-14-1-8(11.5 g, 1.00 eq), THE (280.00 mL), TBAF (14.00 mL, 1.00 eq). The resulting solution was stirred for 3 hr at room temperature. The reaction was then quenched by the addition of 1 L of water. The resulting solution was extracted with 3×500 mL of ethyl acetate and the organic layers combined and dried over anhydrous sodium sulfate. The solids were filtered out. The filtrate was concentrated under vacuum. The crude product was purified by Flash with the following conditions (IntelFlash-1): Column, C18; mobile phase, ACN/H₂O=5/95 increasing to ACN/H₂O=95/5 within 30; Detector, 254. This resulted in 7.8 g (98% yield) of PH-ALIG-14-1-9 as a white solid. LC-MS: m/z 561.20 [M−H]⁻; ¹HNMR: (300 MHz, DMSO-d6) δ 11.32 (s, 1H), 7.66 (d, J=8.1 Hz, 1H), 7.52-7.39 (m, 2H), 7.39-7.20 (m, 7H), 6.96-6.83 (m, 4H), 6.17 (t, J=5.9 Hz, 1H), 5.63 (d, J=8.0 Hz, 1H), 4.63 (t, J=5.6 Hz, 1H), 3.90-3.46 (m, 9H), 3.26 (s, 5H), 3.19-2.98 (m, 2H).

### Preparation of PH-ALIG-14-1-10

Into a 3-L round-bottom flask, was placed PH-ALIG-14-1-9 (7.80 g, 1.00 eq), DCM (300.00 mL), NaHCO₃ (3.50 g, 3 eq). This was followed by the addition of Dess-Martin (7.06 g, 1.2 equiv) with stirring at 0° C., and the resulting solution was stirred for 20 min at 0° C. The resulting solution was stirred for 5 hr at room temperature. The reaction mixture was cooled to 0 degree c. with a water/ice bath. The reaction was then quenched by the addition of 500 mL of NaHCO₃:Na₂S₂O₃=1:1. The resulting solution was extracted with 3×500 mL of ethyl acetate and the organic layers combined and dried over anhydrous sodium sulfate. The solids were filtered out. The filtrate was concentrated under vacuum. The crude product was purified by Flash with the following conditions (IntelFlash-1): Column, C18; mobile phase, ACN/H₂O=5/95 increasing to ACN/H₂O=95/5 within 30; Detector, 254. This resulted in 5.8 g (75% yield) of PH-ALIG-14-1-10 as a white solid. LC-MS: m/z 558.80 [M−H]⁻; ¹HNMR-:(300 MHz, DMSO-d6) δ 11.35-11.22 (m, 1H), 9.43 (s, 1H), 7.75 (d, J=8.1 Hz, 1H), 7.49-7.19 (m, 8H), 6.90 (m, 5H), 6.00 (t, J=5.9 Hz, 1H), 5.66 (m, 1H), 4.40 (m, 1H), 3.75 (s, 7H), 3.70-3.56 (m, 3H), 3.29 (d, J=3.7 Hz, 3H).

### Preparation of PH-ALIG-14-1-11

Into a 250-mL 3-round-bottom flask, was placed THE (150.00 mL), NaH (1.07 g, 60% w, 3.00 equiv). The flask was evacuated and flushed three times with Argon, and the reaction mixture was cooled to −78° C. This was followed by the addition of [[(bis[[(2,2-dimethylpropanoyl)oxy]methoxy]phosphoryl)methyl([(2,2-dimethylpropanoyl)oxy]methoxy)phosphoryl]oxy]methyl 2,2-dimethylpropanoate (14.60 g, 2.6 eq, in 60 m L THF) dropwise with stirring at −78° C. in 10 min, and the resulting solution was stirred for 30 min at −78° C. This was followed by the addition of PH-ALIG-14-1-10 (5.00 g, 1.00 eq, in 50 mL THF) dropwise with stirring at −78° C. in 10 min. The resulting solution was stirred for 4 hr at room temperature. The reaction was then quenched by the addition of 400 mL of NH₄Cl(sat.). The resulting solution was extracted with 3×400 mL of ethyl acetate and the organic layers combined and dried over anhydrous sodium sulfate. The solids were filtered out. The filtrate was concentrated under vacuum. The crude product was purified by Flash with the following conditions (IntelFlash-1): Column, C18; mobile phase, ACN/H₂O=5/95 increasing to ACN/H₂O=95/5 within 30; Detector, 254. This resulted in 7.2 g (crude) of PH-ALIG-14-1-11a solid. LC-MS: m/z:865.10 [M−H]⁻

### Preparation of PH-ALIG-14-1-12

Into a 500-mL round-bottom flask, was placed PH-ALIG-14-1-11 (6.00 g), H₂O (30.00 mL), AcOH (120.00 mL). The resulting solution was stirred for 1 hr at 50 degrees C. The reaction mixture was cooled to 0 degree c. with a water/ice bath. The reaction was then quenched by the addition of 2 L of NaHCO₃ (sat.). The pH value of the solution was adjusted to 7 with NaHCO₃ (sat.). The resulting solution was extracted with 3×500 mL of ethyl acetate and the organic layers combined and dried over anhydrous sodium sulfate. The solids were filtered out. The filtrate was concentrated under vacuum. The crude product was purified by Flash with the following conditions (IntelFlash-1): Column, C18; mobile phase, ACN/H₂O=5/95 increasing to ACN/H₂O=95/5 within 30; Detector, 254. This resulted in 2.6 g (44% yield in two steps) of PH-ALIG-14-1-12 as yellow oil. LC-MS: m/z 587.25 [M+Na]+; ¹HNMR:(300 MHz, DMSO-d6) δ 11.31 (s, 1H), 7.73 (d, J=8.1 Hz, 1H), 6.63 (ddd, J=24.2, 17.2, 4.2 Hz, 1H), 6.14-5.96 (m, 2H), 5.65-5.48 (m, 5H), 5.09 (t, J=5.6 Hz, 1H), 4.17 (s, 1H), 3.65 (d, J=6.1 Hz, 2H), 3.52 (m, 2H), 3.27 (s, 3H), 1.15 (d, J=3.7 Hz, 18H); ³¹PNMR-:(162 MHz, DMSO-d6) δ 17.96.

### Preparation of PH-ALIG-14-1-0

Into a 250-mL 3-necked round-bottom flask, was placed DCM (60.00 mL), DCI (351.00 mg, 1.2 eq), 3-[[bis(diisopropylamino)phosphanyl]oxy]propanenitrile (971.00 mg, 1.3 eq), 4A MS. The flask was evacuated and flushed three times with Argon, and the reaction mixture was cooled to 0° C. This was followed by the addition of PH-ALIG-14-1-12 (1.40 g, 1.00 eq, in 30 mL DCM) dropwise with stirring at 0° C. in 30 second. The resulting solution was stirred for 1 hr at room temperature. The reaction was then quenched by the addition of 50 mL of water. The resulting solution was extracted with 3×50 mL of ethyl acetate and the organic layers combined. The resulting mixture was washed with 3×50 ml of NaCl(sat.). The mixture was dried over anhydrous magnesium sulfate. The solids were filtered out. The filtrate was concentrated under vacuum. The crude product was purified by Prep-Archiral-SFC with the following conditions: Column: Ultimate Diol, 2*25 cm, 5 _{|}^{|}Ìm; Mobile Phase A: CO₂, Mobile Phase B: ACN(0.2% TEA); Flow rate: 50 mL/min; Gradient: isocratic 30% B; Column Temperature(20° C.): 35; Back Pressure(bar): 100; Wave Length: 254 nm; RT1(min): 2.58; Sample Solvent: MeOH-HPLC; Injection Volume: 1 mL; Number Of Runs: 4. This resulted in 1.31 g (65% yield) PH-ALIG-14-1-0 as yellow oil. LC-MS: m/z 763.40 [M−H]⁻; 1HNMR-: (300 MHz, Acetonitrile-d3) δ 9.05 (s, 1H), 7.51 (d, J=8.1 Hz, 1H), 6.64 (dddd, J=23.8, 17.1, 4.8, 1.9 Hz, 1H), 6.23-5.92 (m, 2H), 5.70-5.51 (m, 5H), 4.38 (d, J=4.9 Hz, 1H), 3.96-3.56 (m, 8H), 3.35 (s, 3H), 2.70 (m, 2H), 1.33-1.14 (m, 30H); 31 PNMR-:(Acetonitrile-d3) δ 148.75, 148.53, 16.68.

### Example 3

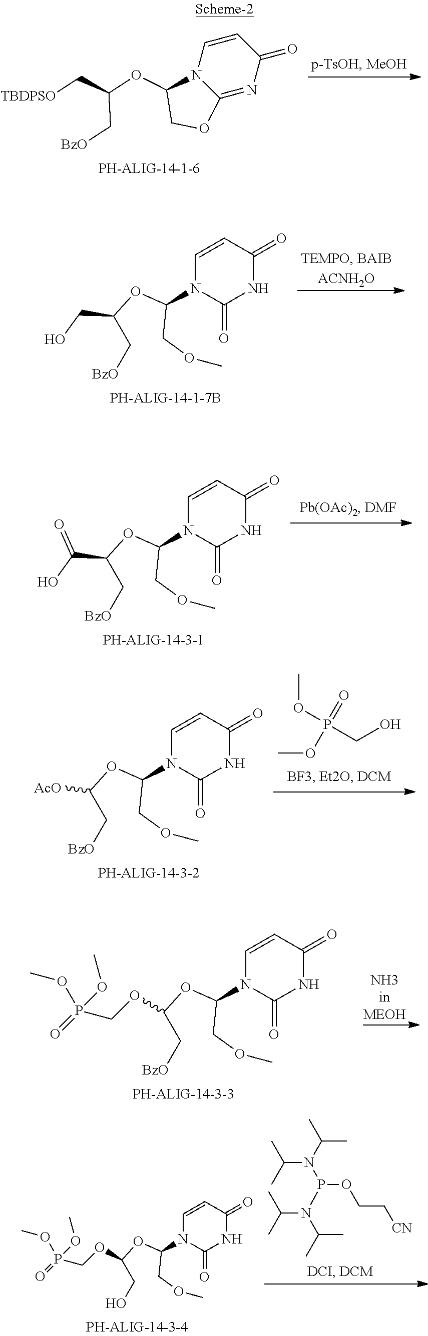

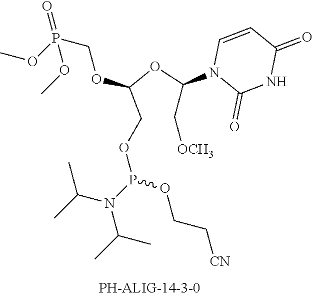 [SMILES: COC[C@@H](O[C@@H](COP(OCCC#N)N(C(C)C)C(C)C)OCP(=O)(OC)OC)n1ccc(=O)[nH]c1=O]

### Preparation of PH-ALIG-14-1-7B

A solution of PH-ALIG-14-1-6 (23 g, 40.300 mmol, 1.00 equiv) and p-TsOH (9.02 g, 52.390 mmol, 1.3 equiv) in MeOH (1000 mL) was stirred for overnight at 40° C. under argon atmosphere. The reaction was quenched with sat. sodium bicarbonate (aq.) at 0 degrees C. The resulting mixture was extracted with EtOAc (2×500 mL). The combined organic layers were washed with water (2×500 mL), dried over anhydrous MgSO₄. After filtration, the filtrate was concentrated under reduced pressure. The residue was purified by reverse flash chromatography with the following conditions: column, C18 silica gel; mobile phase, ACN in water, 10% to 90% gradient in 30 min; detector, UV 254 nm. This resulted in PH-ALIG-14-1-7B (5.3 g, 36.%) as a colorless oil.; LC-MS:(ES, m/z): 365 [M+H]; ¹H-NMR: (300 MHz, DMSO-d6) δ 11.20 (s, 1H), 8.09-7.78 (m, 2H), 7.63-7.50 (m, 2H), 7.51-7.35 (m, 2H), 5.95 (t, J=5.9 Hz, 1H), 5.51 (d, J=8.1 Hz, 1H), 4.73 (t, J=5.7 Hz, 1H), 4.41 (dd, J=11.9, 3.3 Hz, 1H), 4.17 (dd, J=11.9, 6.3 Hz, 1H), 3.69 (dq, J=10.1, 6.8, 6.3 Hz, 1H), 3.48-3.40 (m, 2H), 3.39-3.29 (m, 2H), 3.07 (s, 3H).

### Preparation s of PH-ALIG-14-3-1

Into a 250-mL 3-necked round-bottom flask, was placed PH-ALIG-14-1-7B (7.00 g, 19.212 mmol, 1.00 equiv), ACN (60.00 mL), H₂O (60.00 mL), TEMPO (0.72 g, 4.611 mmol, 0.24 equiv), BAIB (13.61 g, 42.267 mmol, 2.20 equiv). The resulting solution was stirred for 1 overnight at 30° C. The reaction was then quenched by the addition of 200 mL of water/ice. The resulting solution was extracted with 2×200 mL of ethyl acetate, The resulting mixture was washed with 2×200 ml of water. The mixture was dried over anhydrous sodium sulfate and concentrated. The crude product was purified by Flash-Prep-HPLC with the following conditions (IntelFlash-1): Column, C18 silica gel; mobile phase, ACN/H₂O=5/95 increasing to ACN/H₂O=95/5 within 30 min; Detector, UV 254 nm; product was obtained. This resulted in 5 g (68.8%) of PH-ALIG-14-3-1 as a solid. LC-MS:(ES, m/z): 379 [M+H]; ¹H NMR (300 MHz, DMSO-d6) δ 13.24 (s, 1H), 11.31 (d, J=2.2 Hz, 1H), 8.18-7.83 (m, 2H), 7.81-7.63 (m, 2H), 7.61-7.42 (m, 2H), 6.01 (t, J=6.0 Hz, 1H), 5.61 (dd, J=8.0, 2.2 Hz, 1H), 4.72-4.40 (m, 3H), 3.73-3.55 (m, 2H), 3.22 (s, 3H).

### Preparation of PH-ALIG-14-3-2

Into a 250-mL round-bottom flask, was placed PH-ALIG-14-3-1 (4.5 g, 11.894 mmol, 1.00 equiv), DMF (90.00 mL,), Pb(OAc)4 (15.82 g, 35.679 mmol, 3.00 equiv). The resulting solution was stirred overnight at 30° C. The reaction was then quenched by the addition of 200 mL of water/ice. The resulting solution was extracted with 2×200 mL of ethyl acetate The resulting mixture was washed with 2×200 ml of water. The mixture was dried over anhydrous sodium sulfate and concentrated. The crude product was purified by Flash with the following conditions (IntelFlash-1): Column, C18 silica gel; mobile phase, ACN/H₂O=5/95 increasing to ACN/H₂O=95/5 within 30 min; Detector, UV 254 nm; product was obtained. This resulted in 4 g PH-ALIG-14-3-2 as oil; LC-MS:(ES, m/z): 415 [M+Na]+; ¹H NMR (300 MHz, DMSO-d6) δ 11.39 (s, 1H), 7.93 (dd, J=24.2, 7.6 Hz, 2H), 7.75-7.46 (m, 4H), 6.35-6.03 (m, 2H), 5.71-5.47 (m, 1H), 4.60-4.14 (m, 2H), 3.88-3.54 (m, 2H), 3.26 (d, J=6.7 Hz, 3H), 2.03 (d, J=49.7 Hz, 3H).

### Preparation of PH-ALIG-14-3-3

Into a 250-mL 3-necked round-bottom flask purged and maintained with an inert atmosphere of argon, was placed PH-ALIG-14-3-2 (4.00 g, 10.195 mmol, 1.00 eq), DCM (80.00 mL), dimethyl hydroxymethylphosphonate (22.85 g, 163.114 mmol, 16.00 eq), BF₃·Et₂O (28.94 g, 203.91 mmol, 20 eq). The resulting solution was stirred overnight at room temperature. The reaction was then quenched by the addition of 500 mL of water/ice. The resulting solution was extracted with 2×500 mL of ethyl acetate The resulting mixture was washed with 2×500 ml of water. The mixture was dried over anhydrous sodium sulfate and concentrated. The residue was applied onto a silica gel column with dichloromethane/methanol (20/1). This resulted in 2 g (41.5%) of PH-ALIG-14-3-3 as a solid. LC-MS:(ES, m/z): 490 [M+H₂O]+; 1H-NMR (300 MHz, DMSO-d6) δ 11.39 (d, J=5.4 Hz, 1H), 7.96 (dt, J=11.5, 9.3 Hz, 2H), 7.81-7.40 (m, 4H), 6.29-5.98 (m, 1H), 5.56 (dd, J=12.2, 8.1 Hz, 1H), 5.28-4.99 (m, 1H), 4.29 (dp, J=25.1, 5.9 Hz, 2H), 4.16-3.84 (m, 2H), 3.75-3.53 (m, 7H), 3.28 (d, J=12.5 Hz, 2H).

### Preparation of PH-ALIG-14-3-4

Into a 100-mL round-bottom flask, was placed PH-ALIG-14-3-3 (2.00 g, 4.234 mmol, 1.00 equiv), 7M NH₃(g) in THE (20.00 mL) was added. The resulting solution was stirred overnight at 25° C. The resulting mixture was concentrated under vacuum. The crude product was purified by prep-sfc Column: Lux Sum i-Cellulose-5, 3*25 cm, 5 m; Mobile Phase A: CO₂, Mobile Phase B: MeOH(0.1% 2M NH₃-MEOH); Flow rate: 70 mL/min; Gradient: isocratic 50% B; Column Temperature(25° C.): 35; Back Pressure(bar): 100; Wave Length: 220 nm; RT1(min): 3.75; RT2(min): 4.92; Sample Solvent: MeOH:DCM=1:1; Injection Volume: 1 mL; Number Of Runs: 15, This resulted in 330 mg (21.2%) of PH-ALIG-14-3-4 as a solid. 1H-NMR-: (300 MHz, DMSO-d6) δ 11.14 (s, 1H), 7.63 (d, J=8.1 Hz, 1H), 6.06 (t, J=5.9 Hz, 1H), 5.64 (d, J=8.0 Hz, 1H), 4.89 (s, 1H), 4.63 (t, J=5.3 Hz, 1H), 3.98 (d, J=9.8 Hz, 2H), 3.70 (dd, J=10.7, 1.2 Hz, 8H), 3.63 (dd, J=6.0, 3.2 Hz, 1H), 3.29 (s, 3H).

### Preparation of PH-ALIG-14-3-0

To a stirred solution of 3-{[bis(diisopropylamino)phosphanyl]oxy}propanenitrile (324.10 mg, 1.075 mmol, 1.2 equiv) and 1H-imidazole-4,5-dicarbonitrile (126.99 mg, 1.075 mmol, 1.2 equiv) in DCM (10 mL) was added PH-ALIG-14-3-4 (330 mg, 0.9 mmol, 1.00 eq) dropwise at 25° C. under argon atmosphere. The resulting mixture was stirred for 30 min at 25 degrees C. The reaction was quenched with water/ice. The resulting mixture was extracted with EtOAc (2×10 mL). The combined organic layers were washed with water (2×10 mL), dried over anhydrous MgSO₄. After filtration, the filtrate was concentrated under reduced pressure. Column: Ultimate Diol, 2*25 cm, 5 m; Mobile Phase A: CO₂, Mobile Phase B: ACN; Flow rate: 50 mL/min; Gradient: isocratic 30% B; Column Temperature(25° C.): 35; Back Pressure(bar): 100; Wave Length: 254 nm; RT1(min): 3.95; Sample Solvent: ACN; Injection Volume: 1 mL; Number Of Runs: 10, This resulted in PH-ALIG-14-3-0 (349 mg, 68.4%) as a light yellow oil. LC-MS:(ES, m/z): 567.25 [M+H]⁺; 1H-NMR: (300 MHz, DMSO-d6) δ 11.38 (s, 1H), 7.64 (dd, J=8.0, 1.3 Hz, 1H), 6.09 (dt, J=5.8, 3.4 Hz, 1H), 5.65 (dd, J=8.0, 3.2 Hz, 1H), 4.83 (q, J=5.5 Hz, 1H), 4.03 (dt, J=9.7, 2.2 Hz, 2H), 3.83-3.40 (m, 14H), 3.30 (s, 3H), 2.77 (t, J=5.9 Hz, 2H), 1.12 (ddd, J=9.2, 6.7, 1.7 Hz, 12H); ³¹P NMR (DMSO-d6) δ 148.0, 147.6, 23.1

### Example 4

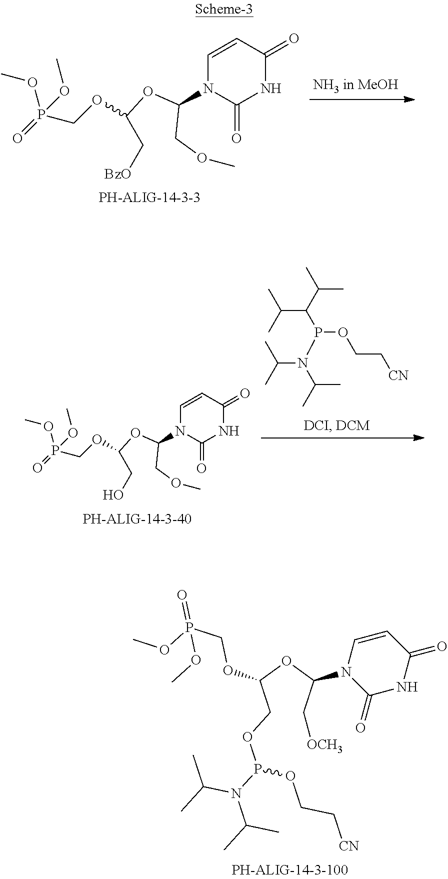

### Preparation of PH-ALIG-14-3-40

Into a 100-mL round-bottom flask, was placed 2 PH-ALIG-14-3-3 (2.00 g, 4.234 mmol, 1.00 equiv), 7M NH₃(g) in THE (20.00 mL) was added. The resulting solution was stirred overnight at 25° C. The resulting mixture was concentrated under vacuum. The crude product was purified by prep-sfc Column: Lux 5 um i-Cellulose-5, 3*25 cm, 5 m; Mobile Phase A: CO₂, Mobile Phase B: MeOH (0.1% 2M NH₃-MeOH); Flow rate: 70 mL/min; Gradient: isocratic 50% B; Column Temperature(° C.): 35; Back Pressure(bar): 100; Wave Length: 220 nm; RT1(min): 3.75; RT2(min): 4.92; Sample Solvent: MeOH:DCM=1:1; Injection Volume: 1 mL; Number Of Runs: 15, This resulted in 320 mg(22.8%) of PH-ALIG-14-3-40 as a solid. 1H-NMR- −14-3-40: (300 MHz, DMSO-d6) δ 11.11 (s, 1H), 7.70 (d, J=8.0 Hz, 1H), 6.03 (t, J=6.1 Hz, 1H), 5.64 (d, J=8.0 Hz, 1H), 4.97 (s, 1H), 4.76 (t, J=5.3 Hz, 1H), 4.07-3.85 (m, 1H), 3.79 (dd, J=13.9, 9.3 Hz, 1H), 3.73-3.55 (m, 9H), 3.41 (d, J=5.0 Hz, 2H), 3.28 (s, 3H).

### Preparation of PH-ALIG-14-3-100

To a stirred solution/mixture of 3-{[bis(diisopropylamino)phosphanyl]oxy}propanenitrile (517.58 mg, 1.717 mmol, 1.2 equiv) and 1H-imidazole-4,5-dicarbonitrile (202.79 mg, 1.717 mmol, 1.2 equiv) in DCM was added PH-ALIG-14-3-40 (527 mg, 1.431 mmol, 1.00 eq.) dropwise at 25° C. under argon atmosphere. The resulting mixture was stirred for 30 min at 25° C. The reaction was quenched with Water/Ice. The resulting mixture was extracted with EtOAc (2×10 mL). The combined organic layers were washed with water (2×10 mL), dried over anhydrous MgSO₄. After filtration, the filtrate was concentrated under reduced pressure. Column: Ultimate Diol, 2*25 cm, 5 m; Mobile Phase A: CO₂, Mobile Phase B: ACN(0.1% DEA)-HPLC--merk; Flow rate: 50 mL/min; Gradient: isocratic 30% B; Column Temperature(° C.): 35; Back Pressure(bar): 100; Wave Length: 254 nm; RT1(min): 4.57; Sample Solvent: ACN; Injection Volume: 1 mL; Number Of Runs: 10 to afford PH-ALIG-14-3-100 (264.8 mg, 31.7%) as a light yellow oil. LC-MS:(ES, m/z): 567.25 [M−H]⁻: 1H NMR (300 MHz, DMSO-d6) δ 13.24 (s, 1H), 11.31 (d, J=2.2 Hz, 1H), 8.18-7.83 (m, 2H), 7.81-7.63 (m, 2H), 7.61-7.42 (m, 2H), 6.01 (t, J=6.0 Hz, 1H), 5.61 (dd, J=8.0, 2.2 Hz, 1H), 4.72-4.40 (m, 3H), 3.73-3.55 (m, 2H), 3.22 (s, 3H); ³¹P NMR (DMSO-d6) δ 148.01, 147.67, 22.8

### Example 5

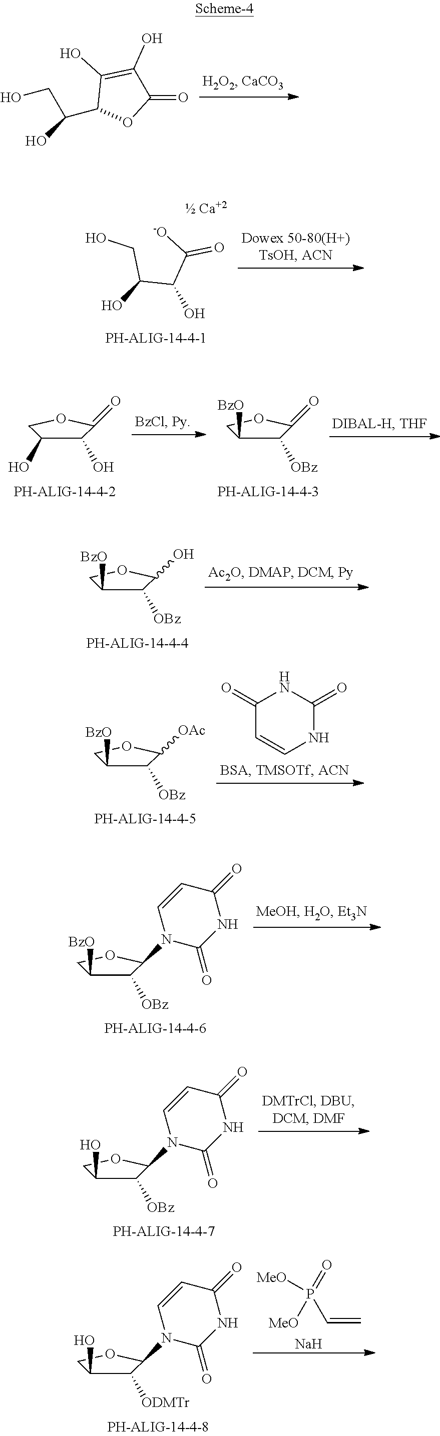

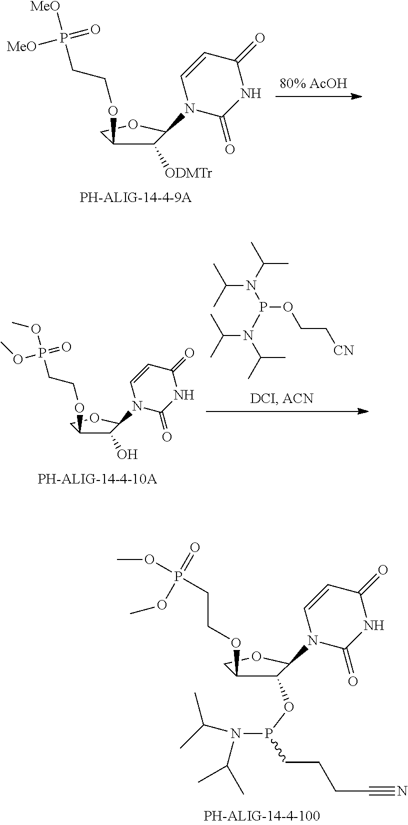

### Preparation of PH-ALIG-14-4-1

To a stirred mixture of ascorbic acid (100.00 g, 567.78 mmol, 1.00 equiv) and CaCO₃(113.0 g, 1129.02 mmol, 2 equiv) in H₂O (1.00 L) was added H₂O₂ (30%)(236.0 g, 6938.3 mmol, 12.22 equiv) dropwise at 0° C. The resulting mixture was stirred overnight at room temperature. The mixture was treat with charcoal and heat to 70 degrees until the no more peroxide was detected. The resulting mixture was filtered, the filter cake was washed with warm water (3×300 mL). The filtrate was concentrated under reduced pressure. The solid was diluted with MeOH (200 mL) and the mixture was stirred for 5 h. The resulting mixture was filtered, the filter cake was washed with MeOH (3×80 mL). The filtrate was concentrated under reduced pressure to afford L-threonate (86 g, 96.6%) as a white crude solid. 1H-NMR-: (300 MHz, Deuterium Oxide) δ 4.02 (dd, J=4.6, 2.4 Hz, 1H), 3.91 (ddt, J=7.6, 5.3, 2.2 Hz, 1H), 3.78-3.44 (m, 2H).

### Preparation of PH-ALIG-14-4-2

Into a 5 L round-bottom flask were added L-threonate (70.00 g, 518.150 mmol, 1.00 equiv) and H₂O (2 L) at room temperature. The residue was acidified to pH=1 with Dowex 50w×8, H(+)Form). The resulting mixture was stirred for 1 h at 70° C. The resulting mixture was filtered, the filter cake was washed with water (2×1 L). The filtrate was concentrated under reduced pressure. The solid was co-evaporated with (2×2 L). Then the solid was diluted with ACN (700.00 mL), and the TsOH(5.35 g, 31.089 mmol, 0.06 equiv) was added. The resulting mixture was stirred for 1 h at 80 degrees C. under air atmosphere. The resulting mixture was filtered, the filter cake was washed with ACN (2×500 mL). The filtrate was concentrated under reduced pressure to PH-ALIG-14-4-2 (70 g, crude) as a yellow oil.

### Preparation of PH-ALIG-14-4-3

To a stirred solution of (PH-ALIG-14-4-2 (70.0 g crude, 593.2 mmol, 1.00 eq.) in pyridine (280.00 mL) was added benzoyl chloride (207.62 g, 1.483 mol, 2.5 equiv) dropwise at 0° C. under argon atmosphere. The resulting mixture was stirred for 1 h at room temperature under argon atmosphere. The reaction was quenched by the addition of sat. NaHCO₃(aq.) (500 mL) at 0 degrees C. The resulting mixture was extracted with CH₂C12 (3×500 mL). The combined organic layers were washed with brine (2×300 mL), dried over anhydrous Na₂SO₄. After filtration, the filtrate was concentrated under reduced pressure. The residue was purified by silica gel column chromatography, eluted with PE/EtOAc to afford (PH-ALIG-14-4-3 (80 g, 41.4%) as an off-white solid. LC-MS: (ES, m/z): 327 [M+H]⁺; 1H-NMR: (300 MHz, CDCl₃) δ 8.18-8.04 (m, 4H), 7.68-7.61 (m, 2H), 7.50 (tt, J=7.1, 1.4 Hz, 4H), 5.96-5.57 (m, 2H), 5.11-5.00 (m, 1H), 4.45-4.35 (m, 1H).

### Preparation of PH-ALIG-14-4-4

To a stirred solution of PH-ALIG-14-4-3 (125 g, 383.078 mmol, 1.00 eq) in THF(1.50 L) was added DIBAL-H (1M)(600 mL, 2 eq) dropwise at −78° C. under argon atmosphere. The resulting mixture was stirred for 1 h at −78 degrees C. under argon atmosphere. Desired product was detected by LCMS. The reaction was quenched with MeOH at 0° C. The resulting mixture was diluted with EtOAc (600 mL). Then the resulting mixture was filtered, the filter cake was washed with EtOAc (3×800 mL). The filtrate was concentrated under reduced pressure. This resulted in PH-ALIG-14-4-4 (73 g, crude) as a colorless solid. LC-MS: (ES, m/z): 392 [M+Na+ACN]+; 1H-NMR-: (400 MHz, Chloroform-d) δ 8.22-7.99 (m, 8H), 7.62 (dtd, J=7.4, 4.4, 2.2 Hz, 4H), 7.48 (td, J=7.8, 2.4 Hz, 8H), 5.87 (d, J=4.3 Hz, 1H), 5.77 (dt, J=6.6, 3.6 Hz, 1H), 5.56 (d, J=4.9 Hz, 2H), 5.50 (t, J=4.3 Hz, 1H), 4.73 (s, 1H), 4.63 (ddd, J=10.4, 7.9, 6.1 Hz, 2H), 4.28 (dd, J=10.3, 3.8 Hz, 1H), 3.99 (dd, J=10.6, 3.2 Hz, 1H).

### Preparation of PH-ALIG-14-4-5

To a stirred solution of (PH-ALIG-14-4-4 (73.00 g, 222.344 mmol, 1.00 equiv) and DMAP (271.63 mg, 2.223 mmol, 0.01 equiv) and pyridine(365.00 mL) in DCM(365.00 mL) were added Ac₂O(24.97 g, 244.6 mmol, 1.1 equiv) dropwise at 0 degrees C. under argon atmosphere. The resulting mixture was stirred for 1 h at room temperature under argon atmosphere. The reaction was quenched with sat. NaHCO₃(aq.) at 0 degrees C. The resulting mixture was extracted with CH₂C2 (3×500 mL). The combined organic layers were washed with sat. CuSO₄ (3×200 mL), dried over anhydrous Na₂SO₄. After filtration, the filtrate was concentrated under reduced pressure. The residue was purified by silica gel column chromatography, eluted with PE/EtOAc to afford PH-ALIG-14-4-5 (60 g, 73%) as a colorless oil. LC-MS: (ES, m/z): 434 [M+Na+ACN]⁺; 1H-NMR: (400 MHz, Chloroform-d) δ 8.17-8.02 (m, 8H), 7.63 (tddd, J=7.9, 6.6, 3.2, 1.6 Hz, 4H), 7.57-7.44 (m, 8H), 6.66 (d, J=4.5 Hz, 1H), 6.40 (s, 1H), 5.83-5.53 (m, 4H), 4.67 (ddd, J=23.4, 10.5, 6.2 Hz, 2H), 4.24 (dd, J=10.5, 3.8 Hz, 1H), 4.19-4.01 (m, 1H), 2.18 (s, 3H), 2.06 (d, J=3.2 Hz, 3H).

### Preparation of PH-ALIG-14-4-6

To a stirred mixture of PH-ALIG-14-4-5 (50.00 g, 135.005 mmol, 1.00 eq) and uracil (15.13 g, 135.005 mmol, 1 eq) in can (500.00 mL) was added BSA (54.81 g, 270.010 mmol, 2 eq) in portions at room temperature under air atmosphere. The resulting mixture was stirred for 1 h at 60° C. under argon atmosphere. After that, the TMSOTf (90.02 g, 405.0 mmol, 3 eq) was added dropwise at 0° C. The resulting mixture was stirred for 2 h at 60° C. under argon atmosphere. The mixture was neutralized to pH=7 with saturated NaHCO₃(aq.) at 0° C. The resulting mixture was extracted with CH₂C2 (3×400 mL). The combined organic layers were washed with brine (2×400 mL), dried over anhydrous Na₂SO₄. After filtration, the filtrate was concentrated under reduced pressure. The residue was purified by silica gel column chromatography, eluted with PE/EtOAc (1:1) to afford PH-ALIG-14-4-6 (43 g, 75.4%) as a white solid. LC-MS: (ES, m/z): [M+H]; 423 464 [M+H+ACN]+; 1H-NMR-: (300 MHz, Chloroform-d) δ 9.08-8.89 (m, 1H), 8.17-7.94 (m, 4H), 7.70-7.43 (m, 7H), 6.19 (d, J=1.9 Hz, 1H), 5.84-5.71 (m, 2H), 5.62 (td, J=3.3, 2.8, 1.4 Hz, 1H), 4.59-4.44 (m, 2H), 4.14 (q, J=7.2 Hz, 1H).

### Preparation of PH-ALIG-14-4-7

A solution of PH-ALIG-14-4-6 (52.00 g, 123.108 mmol, 1 eq) was dissolved in 642 ml of MeOH/H₂O/TEA(5:1:1) at room temperature and heat to reflux until no more starting material was detected(2˜3h). The resulting mixture was concentrated under reduced pressure. The residue was dissolved in EtOAc (600 mL) and the organic layer was extracted with water (5×800 mL). The aqueous layer was concentrated under vacuum to afford PH-ALIG-14-4-7(21 g, crude) as a off-white solid. The crude product was used in the next step directly without further purification. LC-MS-: (ES, m/z): 213 [M−H]⁻; 1 H-NMR: (300 MHz, DMSO-d6) δ 11.26 (s, 1H), 7.68 (d, J=8.1 Hz, 1H), 5.75 (s, 1H), 5.65 (d, J=1.2 Hz, 1H), 5.59 (d, J=8.1 Hz, 1H), 5.39 (s, 1H), 4.10-3.97 (m, 4H).

### Preparation of PH-ALIG-14-4-8

To a stirred mixture of PH-ALIG-14-4-7 (16.00 g, 74.705 mmol, 1.00 equiv) and DBU (22.75 g, 149.409 mmol, 2 equiv) in DCM (80.00 mL) and DMF (200.00 mL) was added DMTr-Cl (7.88 g, 25.680 mmol, 1.1 equiv) dropwise at room temperature under argon atmosphere. The resulting mixture was stirred for 2 h at room temperature under argon atmosphere. The reaction was quenched by the addition of sat. NaHCO₃(aq.) (100 mL) at 0 degrees C. The resulting mixture was extracted with EtOAc (3×60 mL). The combined organic layers were washed with brine (2×50 mL), dried over anhydrous Na₂SO₄. After filtration, the filtrate was concentrated under reduced pressure. The residue was purified by silica gel column chromatography, eluted with PE(0.5% TEA)/EtOAc (2:3) to afford PH-ALIG-14-4-8 (25 g, 64.8%) as a off-white solid.; LC-MS: (ES, m/z): 515 [M−H]⁻; ¹H-NMR: (400 MHz, DMSO-d6) δ 11.33 (s, 1H), 7.57 (d, J=8.1 Hz, 1H), 7.45-7.13 (m, 9H), 6.86 (t, J=8.5 Hz, 4H), 5.94 (d, J=1.7 Hz, 1H), 5.58 (d, J=8.1 Hz, 1H), 5.15 (d, J=2.6 Hz, 1H), 3.97-3.79 (m, 3H), 3.73 (d, J=2.3 Hz, 6H), 3.33 (d, J=2.5 Hz, 1H).

### Preparation of PH-ALIG-14-4-9A

To a stirred solution of PH-ALIG-14-4-8 (6.00 g, 11.616 mmol, 1.00 eq) in THE (240.00 mL) was added NaH (60%) (1.40 g, 35.003 mmol, 3 eq) dropwise at 0° C. under argon atmosphere. The resulting mixture was stirred for 30 min at 0 degrees C. under argon atmosphere. Then the dimethyl ethenylphosphonate (15.81 g, 116.2 mmol, 10.00 eq) was added and the resulting mixture was stirred overnight at room temperature under argon atmosphere. The reaction was quenched with sat. NH₄Cl (aq.) at room temperature. The resulting mixture was extracted with EtOAc (3×100 mL). The combined organic layers were washed with brine (3×80 mL), dried over anhydrous Na₂SO₄. After filtration, the filtrate was concentrated under reduced pressure. The residue was purified by reverse flash chromatography with the following conditions: column, C18 mobile phase, ACN in water, 5% to 95% gradient in 30 min; detector, UV 254 nm to afford PH-ALIG-14-4-9A (3.65 g, 48.15%) as a white solid. LC-MS: (ES, m/z): 675 [M+Na]+; 1H-NMR-: (300 MHz, DMSO-d6) δ 11.39 (s, 1H), 7.44-7.36 (m, 3H), 7.34-7.21 (m, 7H), 6.93-6.83 (m, 4H), 6.08 (d, J=2.0 Hz, 1H), 5.55 (d, J=8.1 Hz, 1H), 4.08 (d, J=11.0 Hz, 1H), 3.92 (d, J=2.0 Hz, 1H), 3.82-3.71 (m, 7H), 3.57 (dd, J=10.9, 3.6 Hz, 6H), 3.30-3.23 (m, 1H), 3.06-2.86 (m, 2H), 1.96 (dt, J=18.1, 7.1 Hz, 2H).

### Preparation of PH-ALIG-14-4-10A

A solution of PH-ALIG-14-4-9A (2.80 g, 4.3 mmol, 1.00 equiv) in AcOH(12.00 mL) and H₂O(3.00 mL) was stirred for overnight at room temperature under air atmosphere. The reaction was quenched with sat. NaHCO₃(aq.) at 0 degrees C. The resulting mixture was washed with 3×20 mL of CH₂CO₂. The product in the water layer. The water layer was concentrated under reduced pressure. The product was purified by Prep-SFC with the following conditions (Prep SFC80-2): Column, Green Sep Basic, 3*15 cm; mobile phase, CO₂(70%) and IPA(0.5% 2M NH₃-MeOH)(30%); Detector, UV 254 nm; product was obtained. This resulted in 870 mg (57.89%) of PH-ALIG-14-4-10A as a white solid. LC-MS: (ES, m/z): 351 [M+Na]+; 1H-NMR-: (300 MHz, DMSO-d6) δ 11.28 (s, 1H), 7.56 (d, J=8.1 Hz, 1H), 5.86 (d, J=4.4 Hz, 1H), 5.65 (d, J=1.6 Hz, 1H), 5.56 (d, J=8.1 Hz, 1H), 4.17 (d, J=10.1 Hz, 1H), 4.10 (d, J=4.3 Hz, 1H), 4.00 (dd, J=10.1, 3.9 Hz, 1H), 3.87 (dt, J=4.1, 1.3 Hz, 1H), 3.72-3.49 (m, 8H), 2.08 (dd, J=7.1, 2.8 Hz, 1H), 2.05-1.96 (m, 1H).

### Preparation of PH-ALIG-14-4-100

Into a 250 mL 3-necked round-bottom flask were added Molecularsieve and ACN (30.00 mL) at room temperature. The resulting mixture was stirred for 10 min at room temperature under argon atmosphere. Then to the stirred solution were added 3-[[bis(diisopropylamino)phosphanyl]oxy]propanenitrile (1058.46 mg, 3.512 mmol, 1.5 equiv) and DCI (359.12 mg, 3.043 mmol, 1.30 equiv). Then the dimethyl PH-ALIG-14-4-10A (820.00 mg, 2.341 mmol, 1.00 equiv) in 30 mL ACN was added dropwise at room temperature under argon atmosphere. The resulting mixture was stirred for 1 h at room temperature under argon atmosphere. The resulting mixture was diluted with CH₂Cl₂ (60 mL). The combined organic layers were washed with water (3×40 mL) after filtration, dried over anhydrous MgSO₄. After filtration, the filtrate was concentrated under reduced pressure. The residue was purified by Prep-TLC (0.5% TEA in PE/10% EtOH in EtOAc 1:9) to afford PH-ALIG-14-4-100 (800 mg, 62.1%) as a colorless oil. LC-MS: (ES, m z): 549 [M−H]⁻; 1H-NMR: (300 MHz, DMSO-d6) δ 11.34 (s, 1H), 7.61 (dd, J=8.1, 1.7 Hz, 1H), 5.80 (dd, J=15.0, 1.8 Hz, 1H), 5.60 (d, J=8.1 Hz, 1H), 4.48-4.23 (m, 2H), 4.17-3.98 (m, 2H), 3.88-3.73 (m, 2H), 3.72-3.51 (m, 10H), 2.79 (q, J=5.9 Hz, 2H), 2.07 (dtt, J=17.9, 7.1, 3.2 Hz, 2H), 1.15 (ddd, J=6.3, 3.8, 2.1 Hz, 12H); ³¹P NMR (DMSO-d6) δ 149.71, 149.35, 30.85, 30.75

### Example 6

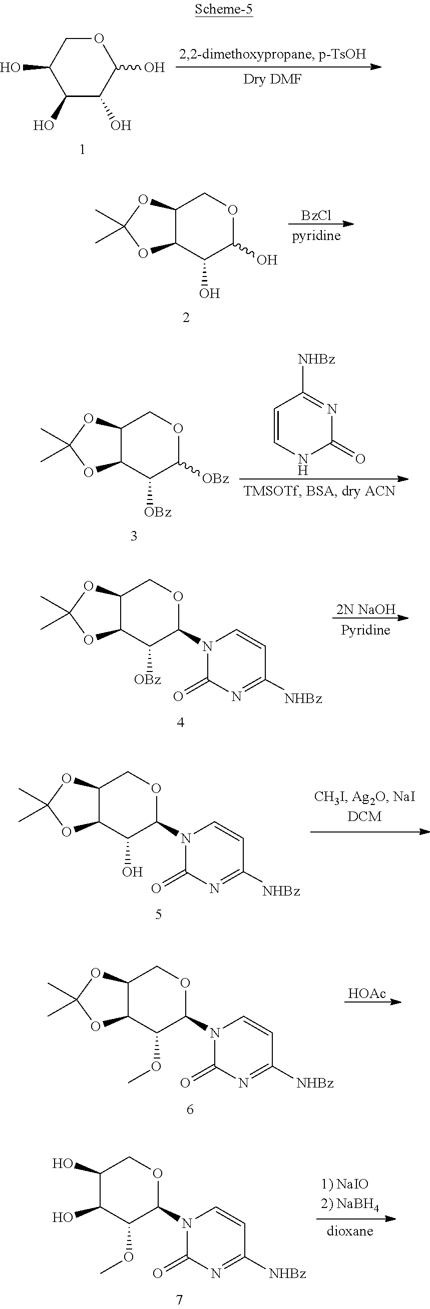

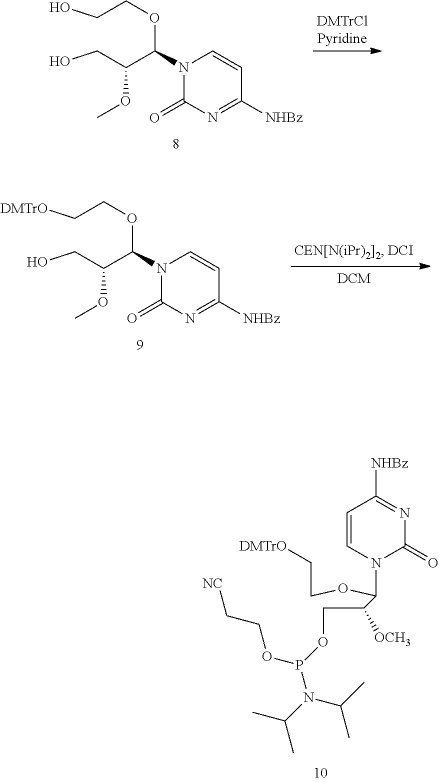

Preparation of 2: (J. Chem. Soc., Perkin Trans. 1, 1992, 1943-1952) To a solution of 1 (150.0 g, 1.0 mol) in DMF (2.0 L) was added 2, 2-dimethoxypropane (312.0 g, 3.0 mol) and p-TsOH (1.7 g, 10.0 mmol), then the reaction mixture was stirred at r.t. for 4 h, after the reaction, the solvent was concentrated to give the crude products which was used directly to next step.

Preparation of 3: (J. Chem. Soc., Perkin Trans. 1, 1992, 1943-1952) To a solution of 2 (190.0 g, 1.0 mol) in pyridine (2.0 L) was added BzCl (560.0 g, 4.0 mol) then the reaction mixture was stirred at r.t. for 2 h, after the reaction, the reaction mixture was poured into the ice water, EA was added for extraction, and the organic phase was washed with brine, dried over Na₂SO₄ and concentrated to give the crude product which was purified by silica gel column (EA:PE=1:5 to 1:1) to give 3 (350.0 g, 87.9% yield), ESI-LCMS: m/z=421.2 [M+Na]⁺.

Preparation of 4: (J. Chem. Soc., Perkin Trans. 1, 1992, 1943-1952) to a solution of 3 (240.0 g, 815.5 mmol) in MeCN (3.0 L) was added N-(2-oxo-1H-pyrimidin-4-yl) benzamide (193.0 g, 897.0 mmol) and BSA (496.6 g, 2.4 mol). then the reaction mixture was stirred at 50° C. for 30 min, then the reaction mixture was cooled to 0° C., and the TMSOTf (271.5 g, 1.2 mol) was added into the mixture at 0° C., then the reaction mixture was stirred at 70° C. for 2 h, after the reaction, the solvent was concentrated to give an oil, then the oil was poured into the solution of NaHCO₃ maintaining the mixture was slightly alkaline, EA was added for extraction, and the organic phase was washed with brine, dried over Na₂SO₄ and concentrated to give the crude product which was purified by silica gel column (EA:PE=1:3 to 1:1) to give 4 (180.0 g, 44.9% yield). ESI-LCMS: m/z=491.2 [M+H]⁺; ¹H NMR (400 MHz, DMSO-d6) δ 11.19 (s, 1H), 8.20 (d, J=7.6 Hz, 1H), 8.01-7.84 (m, 4H), 7.73-7.57 (m, 2H), 7.50 (dt, J=10.4, 7.7 Hz, 4H), 7.40 (d, J=7.4 Hz, 1H), 6.03 (d, J=9.4 Hz, 1H), 5.33 (dd, J=9.4, 7.3 Hz, 1H), 4.66 (dd, J=7.3, 5.3 Hz, 1H), 4.45-4.35 (m, 2H), 4.22 (dd, J=13.7, 2.5 Hz, 1H), 1.58 (s, 3H), 1.34 (s, 3H).

Preparation of 5: To a solution of 4 (78.0 g, 158.7 mmol) in pyridine (800.0 mL) was added a solution of NaOH (6.3 g, 158.7 mmol) in a mixture solvent of H₂O and MeOH (4:1, 2N), Then the reaction mixture was stirred at 0° C. for 20 min, LC-MS and TLC show that the raw material was disappeared, then the mixture was pour into a solution of NH₄Cl, EA was added for extraction, and the organic phase was washed with brine, dried over Na₂SO₄ and concentrated to give the crude product, which was purified by silica gel column (DCM:MeOH=30:1 to 10:1) to give 5 (56.0 g, 91.0% yield). ESI-LCMS: m/z=388.1 [M+H]; ¹H NMR (400 MHz, DMSO-d6) δ 11.29 (s, 1H), 8.16 (d, J=7.6 Hz, 1H), 8.08-7.99 (m, 2H), 7.67-7.60 (m, 1H), 7.53 (t, J=7.6 Hz, 2H), 7.35 (d, J=7.6 Hz, 1H), 5.63 (d, J=6.1 Hz, 1H), 5.51 (d, J=9.5 Hz, 1H), 4.35-4.13 (m, 3H), 3.78 (dt, J=9.6, 6.5 Hz, 1H), 3.19 (d, J=5.1 Hz, 1H), 1.53 (s, 3H), 1.32 (s, 3H).

Preparation of 6: To a solution of 5 (15.0 g, 38.7 mmol) in DCM (200.0 mL) was added Ag₂O (35.8 g, 154.8 mmol), CH₃I (54.6 g, 387.2 mmol) and NaI (1.1 g, 7.7 mmol), then the reaction mixture was stirred at r.t. overnight, after the reaction, filtrate was obtained through filtration, and the filtrate concentrated the solvent to obtain the product 6 (13.0 g, 75.2% yield,). ESI-LCMS: m/z=402.30 [M+H]*; ¹H NMR (400 MHz, DMSO-d6) δ 11.30 (s, 1H), 8.22 (s, 1H), 8.00 (d, J=7.6 Hz, 2H), 7.71-7.20 (m, 4H), 5.56 (d, J=9.3 Hz, 1H), 4.33 (t, J=6.1 Hz, 1H), 4.26 (dd, J=6.2, 2.1 Hz, 1H), 4.20 (d, J=13.5 Hz, 1H), 3.98 (dd, J=13.5, 2.5 Hz, 1H), 3.66 (dd, J=9.3, 6.6 Hz, 1H), 3.34 (s, 3H), 1.57 (s, 3H), 1.32 (s, 3H).

Preparation of 7: To a solution of 6 (12.0 g, 29.9 mmol) was added CH₃COOH (120.0 mL), then the mixture was stirred at r.t. for 2 h, LC-MS and TLC showed that the raw material was disappeared, then the solvent was concentrated to get the crude product 7 (10.0 g, 83.3% yield,). ESI-LCMS: m/z=362.1 [M+H]⁺.

Preparation of 8: To a solution of 7 (10.0 g, 24.9 mmol) in dioxane:H₂O=3:1 (120.0 mL) was added NaIO4(8.8 g, 41.5 mmol), then the reaction mixture was stirred at r.t. for 2 h, LC-MS and TLC showed that the raw material was disappeared, then the reaction mixture was cooled to 0° C., and NaBH₄ (2.4 g, 41.5 mmol) was added into the mixture and stirred at 0° C. for 0.5 h, LC-MS and TLC showed that the raw material was disappeared, then NH₄C1 was added into the mixture to adjust pH to be slightly alkaline, and concentrated to give the crude product, which was purified by silica gel column (PE:EA=5:1 to 1:1) to give 8 (8.0 g, 79.5% yield). ESI-LCMS: m/z=364.1 [M+H]⁺; ¹H NMR (400 MHz, DMSO-d6) δ 11.26 (s, 1H), 8.14 (d, J=7.5 Hz, 1H), 8.07-7.94 (m, 2H), 7.67-7.59 (m, 1H), 7.52 (t, J=7.6 Hz, 2H), 7.37 (s, 1H), 5.91 (d, J=6.0 Hz, 1H), 4.77 (t, J=5.6 Hz, 1H), 4.70 (t, J=5.1 Hz, 1H), 3.70 (ddd, J=11.5, 5.0, 2.5 Hz, 1H), 3.57-3.39 (m, 6H), 3.31 (s, 3H).

Preparation of 9: To a solution of 8 (4.0 g, 11.0 mmol) in pyridine (50.0 mL) was added DMTrCl (5.5 g, 16.5 mmol), then the reaction mixture was stirred at r.t. for 2 h, LC-MS showed that the raw material was 20.0% and The ratio of product to by-product was 3.5:1. then the solvent was concentrated to get residue which was purified by silica gel column to give the purified products and by-products was 5 g in total, then the product was purified by SFC to get 9 (3.0 g, 40.9% yield,). ESI-LCMS: m/z=666.2 [M+H]⁺; ¹H NMR (400 MHz, DMSO-d6) δ 11.33 (s, 1H), 8.20 (d, J=7.4 Hz, 1H), 8.04 (d, J=7.7 Hz, 2H), 7.64 (t, J=7.4 Hz, 1H), 7.53 (t, J=7.6 Hz, 2H), 7.40 (d, J=7.8 Hz, 3H), 7.36-7.18 (m, 7H), 6.89 (d, J=8.4 Hz, 4H), 5.96 (d, J=5.7 Hz, 1H), 4.79 (t, J=5.7 Hz, 1H), 3.73 (s, 6H), 3.66-3.46 (m, 4H), 3.37 (s, 3H), 3.16 (ddd, J=10.1, 7.1, 3.0 Hz, 1H), 3.04 (dt, J=10.9, 3.4 Hz, 1H), 2.08 (s, 1H).

Preparation of 10: To a solution of 9 (2.8 g, 4.2 mmol) in DCM (30.0 mL) was added CEP[N(iPr)2]2 (1.3 g, 4.2 mmol) and DCI (601.2 mg, 5.1 mmol). The mixture was stirred at r.t. for 1 h. LC-MS showed 9 was consumed completely. The solution was washed with a solution of NaHCO₃ twice and washed with brine and dried over Na₂SO₄. Then concentrated to give a residue which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=I/O within 20.0 min, the eluted product was collected at CH₃CN/H₂O (0.5% NH₄HCO₃)=90/10; Detector, UV 254 nm. This resulted in to give 10 (2.8 g, 76.8% yield,). ESI-LCMS: m/z=866.2 [M+H]; ¹H NMR (400 MHz, DMSO-d6) δ 11.34 (s, 1H), 8.22 (d, J=7.4 Hz, 1H), 8.09-7.98 (m, 2H), 7.64 (t, J=7.4 Hz, 1H), 7.53 (t, J=7.6 Hz, 2H), 7.45 (d, J=7.3 Hz, 1H), 7.39 (d, J=7.5 Hz, 2H), 7.31 (t, J=7.6 Hz, 2H), 7.24 (t, J=9.1 Hz, 5H), 6.89 (d, J=8.8 Hz, 4H), 5.96 (d, J=6.1 Hz, 1H), 4.02-3.86 (m, 1H), 3.84-3.63 (m, 11H), 3.56 (dtq, J=13.3, 6.6, 3.5, 3.1 Hz, 3H), 3.37 (s, 2H), 3.16 (ddd, J=10.0, 6.8, 3.3 Hz, 1H), 3.04 (ddd, J=10.7, 5.5, 3.0 Hz, 1H), 2.75 (td, J=5.9, 2.3 Hz, 2H), 1.18-1.07 (m, 12H); ³¹P NMR (DMSO-d6) δ 148.02 (d, J=12.0 Hz).

### Example 7

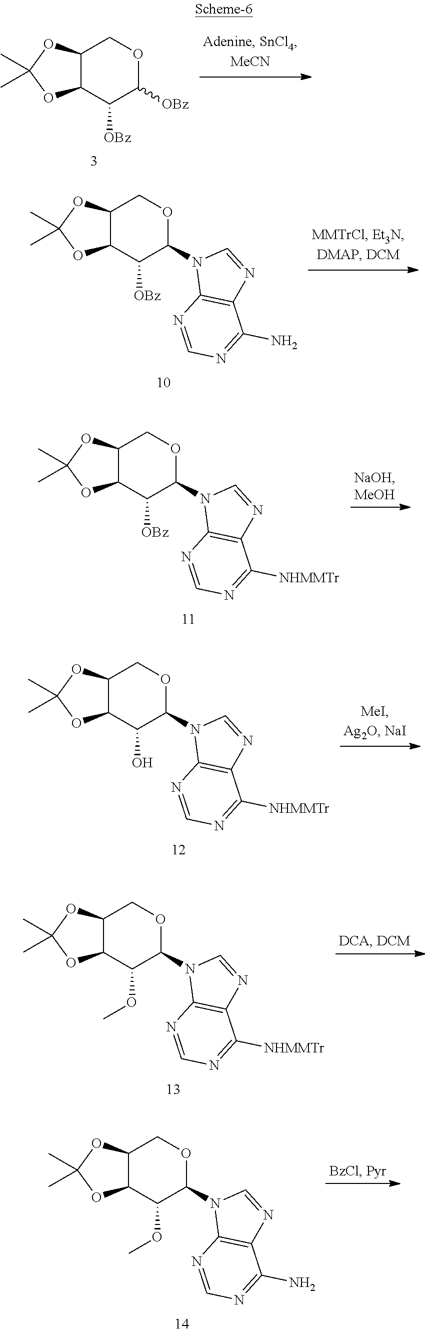

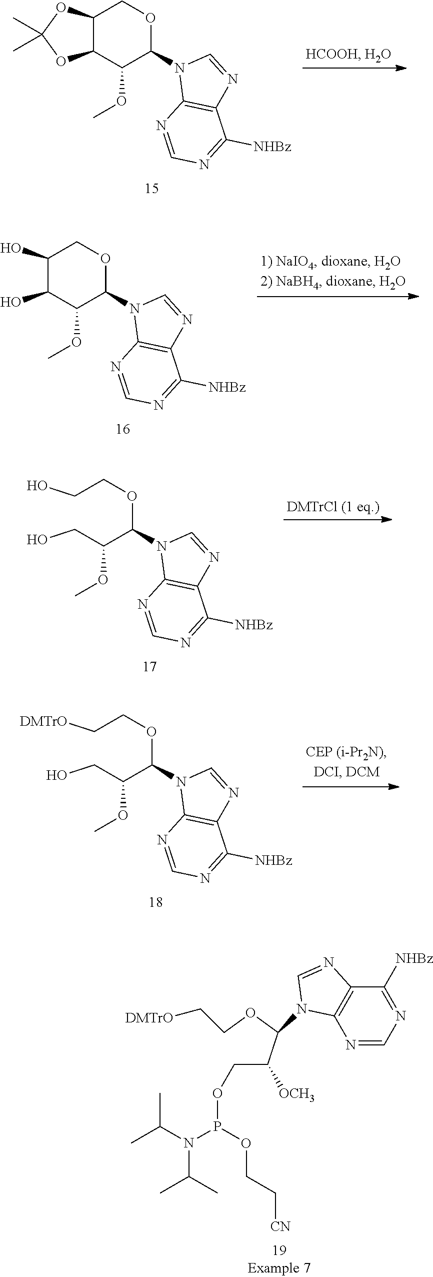

Preparation of 10: To the solution of 3 (200.0 g, 0.5 mol) in ACN (2000.0 mL) was added a solution of SnCl₄ in DCM (1000.0 mL) at 0° C. under N₂, and the reaction mixture was stirred at 0° C. for 4 h under N₂ atmosphere. Then the reaction solution was poured into saturated sodium bicarbonate solution, the resulting product was extracted with EA (3*500.0 mL). The combined organic layer was washed with water and brine, dried over Na₂SO₄, and concentrated to give the crude, which was purified by silica gel column (PE:EA=5:1 to 0:1) to give 10 (65.0 g, 31.4% yield) as a white solid. ESI-LCMS: m/z=412.0 [M+H]⁺; ¹H NMR (400 MHz, DMSO-d6) δ 8.27 (s, 1H), 8.09 (s, 1H), 7.74-7.60 (m, 2H), 7.59-7.57 (m, 1H), 7.44-7.40 (m, 2H), 7.24 (s, 2H), 5.90 (d, J=9.6 Hz, 1H), 5.73 (dd, J=7.4 Hz, 1H), 4.63 (t, 1H), 4.50-4.30 (m, 2H), 4.21 (dd, J=13.6 Hz, 1H), 1.61 (s, 3H), 1.35 (s, 3H).

Preparation of 11: To a solution of 10 (40.0 g, 97.3 mmol) in DCM (500.0 mL) was added Et₃N (30.0 g, 297.0 mmol) and DMAP (1.2 g, 9.8 mmol) at r.t.. The reaction mixture was replaced with N₂ over 3 times, then MMTrCl (45.0 g, 146.1 mmol) was added to the mixture. The reaction mixture was stirred at r.t. overnight. TLC and LC-MS showed that 10 was consumed, and the reaction mixture was added to an aqueous solution of NaHCO₃ in ice-water. Then extracted product with EA, washed the organic phase with brine, and dried the organic phase over Na₂SO₄, then concentrated to get 11 (66.5 g,) as a crude, used next step directly.

Preparation of 12: To a solution of 11 (66.5 g, 97.3 mmol) in pyridine (600.0 mL) was added 2N NaOH (H₂O:MeOH=4:1) (200.0 mL) at r.t.. Then the reaction mixture was stirred at 0° C. for 30 min, LC-MS and TLC showed that the raw material was disappeared, then the mixture was poured into a solution of NH₄Cl, EA was added for extraction, and the organic phase was washed with brine, dried over Na₂SO₄ and concentrated to give the crude product which was purified by silica gel column (EA:PE=1:5 to 1:1) to give 12 (50.0 g, 88.7% yield for two step). ESI-LCMS: m/z=580.4 [M+H]⁺; ¹H NMR (400 MHz, DMSO-d6) δ 8.44 (s, 1H), 7.92 (s, 1H), 7.36-7.16 (m, 13H), 6.89-6.80 (m, 2H), 5.59 (d, J=6.0 Hz, 1H), 5.35 (d, J=9.6 Hz, 1H), 4.32-4.12 (m, 4H), 4.08-3.95 (m, 3H), 3.72 (s, 3H), 1.99 (s, 3H), 1.54 (s, 3H), 1.32 (s, 3H), 1.17 (t, J=7.1 Hz, 3H).

Preparation of 13: To a solution of 12 (46.0 g, 79.4 mmol) in CH₃I (200.0 mL) was added Ag₂O (36.6 g, 158.4 mmol) and NaI (6.0 g, 42.5 mmol), then the reaction mixture was stirred at r.t. for 4 h, then the reaction mixture was filtrated and concentrated the solvent to obtain the product 13 (46.0 g, 97.6% yield), used next step directly. ESI-LCMS: m/z=594.3 [M+H]⁺.

Preparation of 14: To a stirred solution of DCA (22.5 mL) in DCM (750.0 mL) was added 13 (46.0 g, 77.5 mmol) and Et₃Si (185.0 mL) at r.t.. And the reaction mixture was stirred at r.t. for 12 h. The reaction solution was evaporated to dryness under reduced pressure to give a residue, which was slurry with a solution of NaHCO₃ (50.0 mL) to get 14 (19.0 g, 76% yield), which was used next step directly.

Preparation of 15: To a solution of 14 (16.0 g, 49.7 mmol) in pyridine (200.0 mL) was added BzCl (9.0 g, 64.7 mmol) at 0° C. Then the reaction mixture was stirred at r.t. for 2 h. LC-MS showed 6 was consumed completely, then the mixture was cooled to 0° C., and a solution of NaOH in MeOH and H₂O (2 N, 50.0 mL) was added into the reaction mixture, and the mixture was stirred for 1 h at 0° C., then the mixture was poured into a solution of NH₄Cl. The product was extracted with EA (300.0 mL) and the organic layer was washed with brine and dried over Na₂SO₄. Then the organic layer was concentrated to give a residue, which was purified by slurry with PE:EA (8:1, 900.0 mL) to get 15 (20.0 g, 95.0% yield). ESI-LCMS: m/z=426.2 [M+H]⁺; 1H NMR (400 MHz, DMSO-d6) δ 11.21 (s, 1H), 8.77-8.69 (m, 2H), 8.06 (d, J=7.6 Hz, 2H), 7.65 (t, J=7.4 Hz, 1H), 7.56 (t, J=7.6 Hz, 2H), 7.34-7.23 (m, 4H), 7.23-7.12 (m, 5H), 6.89-6.80 (m, 4H), 5.90 (d, J=7.9 Hz, 1H), 4.36-4.29 (m, 1H), 4.06 (t, J=8.8 Hz, 1H), 3.92 (dd, J=25.0, 6.9 Hz, OH), 3.72 (d, J=1.0 Hz, 7H), 3.59 (dt, J=10.4, 6.6 Hz, 1H), 3.24 (s, 3H), 2.97 (d, J=7.7 Hz, 1H), 2.76 (q, J=5.5 Hz, 2H), 1.14 (dd, J=9.2, 5.7 Hz, 12H).

Preparation of 16: To a mixture solution of HCOOH (180.0 mL) and H₂O (20.0 mL) was added 15 (19.0 g, 44.7 mmol). The reaction mixture was stirred at r.t. for 4 h. LC-MS showed 15 was consumed completely. Then the reaction mixture was concentrated to give a residue which was purified by slurry with MeOH (100.0 mL) to get 16 (16.0 g, 92.7% yield) as a white solid. ESI-LCMS: m/z=385.9 [M+H]⁺; 1H NMR (400 MHz, DMSO-d6) δ 11.21 (s, 1H), 8.77 (d, J=1.2 Hz, 2H), 8.09-8.02 (m, 2H), 7.70-7.61 (m, 1H), 7.56 (t, J=7.6 Hz, 2H), 5.56 (d, J=9.2 Hz, 1H), 5.21 (d, J=6.1 Hz, 1H), 4.94 (d, J=4.5 Hz, 1H), 4.18 (t, J=9.1 Hz, 1H), 4.09 (q, J=5.2 Hz, 1H), 3.88-3.71 (m, 4H), 3.21-3.14 (m, 6H).

Preparation of 17: To a solution of 16 (16.0 g, 41.4 mmol) in dioxane (200.0 mL) was added H₂O (32.0 mL), and NaIO4 (9.7 g, 45.5 mmol), then the reaction mixture was stirred at r.t. for 1 h, LC-MS and TLC showed that the raw material was disappeared, then the reaction mixture was cooled to 0° C., and NaBH₄ (1.7 g, 45.5 mmol) was added into the mixture and stirred at 0° C. for 0.5 h, LC-MS and TLC showed that the intermediate state was disappeared, then the NH₄Cl was added into the mixture to adjust pH to be slightly alkaline, and concentrated at r.t. to give the crude product which was purified by silica gel column (DCM:MeOH=20:1 to 8:1) to give 17 (16.0 g, 99.5% yield). ESI-LCMS: m/z=388.0 [M+H]⁺; ¹H NMR (400 MHz, DMSO-d6) δ 11.18 (s, 1H), 8.75 (s, 1H), 8.67 (s, 1H), 8.09-7.99 (m, 2H), 7.65 (t, J=7.4 Hz, 1H), 7.56 (t, J=7.6 Hz, 2H), 5.90 (d, J=7.6 Hz, 1H), 4.88 (t, J=5.7 Hz, 1H), 4.67 (t, J=5.5 Hz, 1H), 4.08-3.98 (m, 2H), 3.78 (ddd, J=12.1, 5.2, 3.1 Hz, 1H), 3.68-3.39 (m, 4H), 3.36 (s, OH), 3.20 (s, 3H), 1.99 (s, 1H), 1.17 (t, J=7.1 Hz, 1H).

Preparation of 18: To a solution of 17 (12.0 g, 31.0 mmol) in pyridine (50.0 mL) was added DMTrCl (11.5 g, 34.1 mmol), then the reaction mixture was stirred at r.t. for 2 h, LC-MS showed that the raw material was 15.0% remained and the ratio of product to by-product was 3.5:1. Then the reaction solution was poured into ice-water, and extracted with EA, wished with brine, dried over Na₂SO₄, filtered and concentrated to get residue which was purified by silica gel column to give the purified product and by-product were 13.0 g in total, then 4.0 g crude was purified by SFC to get 18 (3.3 g, 15.4% yield,). ESI-LCMS: m/z=690.3 [M+H]⁺; ¹H NMR (400 MHz, DMSO-d6) δ 11.21 (s, 1H), 8.75 (s, 1H), 8.69 (s, 1H), 8.10-8.03 (m, 2H), 7.70-7.61 (m, 1H), 7.56 (t, J=7.6 Hz, 2H), 7.35-7.12 (m, 9H), 6.90-6.80 (m, 4H), 5.94 (d, J=7.5 Hz, 1H), 4.88 (t, J=5.6 Hz, 1H), 4.36 (t, J=5.1 Hz, 1H), 4.11 (dt, J=7.4, 3.6 Hz, 1H), 3.82 (ddd, J=11.9, 5.1, 3.1 Hz, 1H), 3.72 (d, J=1.3 Hz, 7H), 3.64 (ddd, J=11.9, 6.2, 4.2 Hz, 1H), 3.45 (qd, J=7.0, 4.9 Hz, 2H), 3.24 (s, 3H), 3.09 (ddd, J=9.9, 6.4, 3.2 Hz, 1H), 2.97 (ddd, J=9.9, 5.7, 3.2 Hz, 1H), 1.23 (s, OH), 1.06 (t, J=7.0 Hz, 1H).

Preparation of 19: To a suspension of 18 (3.3 g, 4.8 mmol) in DCM (40.0 mL) was added DCI (0.5 g, 4.0 mmol) and CEP[N(iPr)2]2 (1.6 g, 5.3 mmol). The mixture was stirred at r.t. for 0.5 h. LC-MS showed 10 was consumed completely. The solution was washed with a solution of NaHCO₃ twice and washed with brine and dried over Na₂SO₄. Then concentrated to give a residue which was purified by Flash-Prep-HPLC with the following conditions (IntelFlash-1): Column, C₁₈ silica gel; mobile phase, CH₃CN/H₂O (0.5% NH₄HCO₃)=1/1 increasing to CH₃CN/H₂O (0.5% NH₄HCO₃)=I/O within 20 min, the eluted product was collected at CH₃CN/H₂O (0.5% NH₄HCO₃)=I/O; Detector, UV 254 nm. This resulted in to give 19 (3.0 g, 3.9 mmol, 81.2% yield) as a white solid. ESI-LCMS: m/z=765.3 [M+H]⁺; ¹H NMR (400 MHz, DMSO-d6) δ 11.22 (s, 1H), 8.80-8.71 (m, 2H), 8.11-8.04 (m, 2H), 7.65 (t, J=7.3 Hz, 1H), 7.56 (t, J=7.5 Hz, 2H), 7.36-7.24 (m, 4H), 7.24-7.15 (m, 5H), 6.89-6.82 (m, 4H), 5.92 (d, J=7.7 Hz, 1H), 4.34 (dt, J=7.5, 3.5 Hz, 1H), 4.08 (ddd, J=10.7, 7.3, 2.7 Hz, 1H), 4.03-3.89 (m, 1H), 3.80-3.72 (m,10H), 3.67-3.53 (m, 2H), 3.47 (dp, J=10.5, 3.4 Hz, 1H), 3.26 (s, 3H) 3.11 (ddd, J=10.3, 6.2, 3.5 Hz, 1H), 3.00 (q, J=6.6, 5.2 Hz, 1H), 2.77 (q, J=5.6 Hz, 2H), 2.08 (s, 1H), 1.15 (t, J=7.0 Hz, 12H).; ³¹P NMR (162 MHz, DMSO-d6) δ 148.30, 147.99.

### Example 8

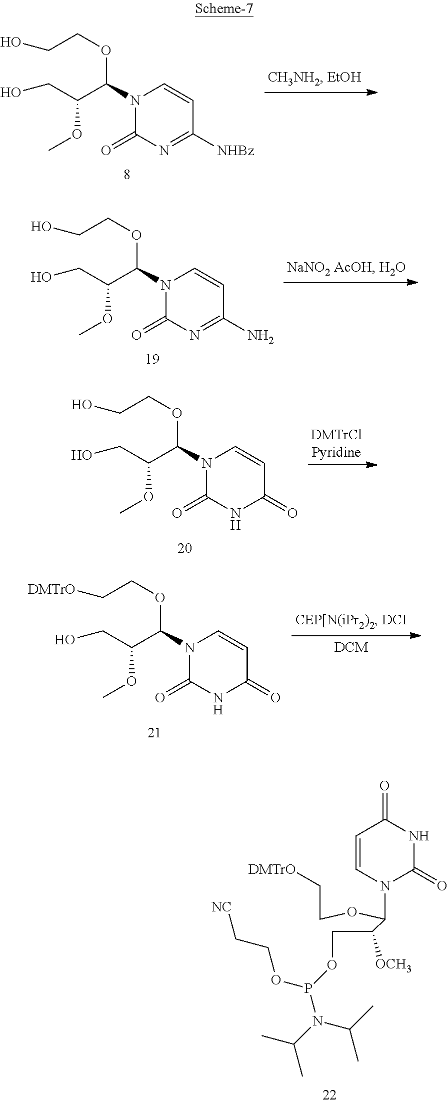

Preparation of 19: To a solution of 8 (8.0 g, 22.0 mmol) in EtOH (50.0 mL) was added a solution of CH₃NH₂ (50.0 mL), then the reaction mixture was stirred at r.t. for 4 h, after the reaction, the solvent was concentrated to give the crude, which was added into a mixture solvent of EA (20.0 mL) and PE (10.0 mL), then the mixture was stirred for 30 min and filtered to get 19 (5.5 g, 96.5% yield), which was used directly to next step.

Preparation of 20: (J. Chem. Soc., Perkin Trans. 1, 1992, 1943-1952) To a solution of 19 (5.0 g, 19.3 mmol) in H₂O (50.0 mL) and AcOH (50.0 mL) was added NaNO₂ (65.0 g, 772.0 mmol), then the reaction mixture was stirred at r.t. for 2 h, after the reaction, the reaction mixture was concentrated to give the crude product which was purified by silica gel column (DCM:MeOH=20:1 to 6:1) and MPLC (ACN:H₂O=0:100 to 10:90) to give 20 (3.0 g, 59.6% yield). ESI-LCMS: m/z=261.2 (M+H)*; ¹H NMR (400 MHz, DMSO-d6) δ 11.29 (s, 1H), 7.66 (d, J=8.0 Hz, 1H), 5.67 (dd, J=17.5, 7.6 Hz, 2H), 4.74 (d, J=36.0 Hz, 2H), 3.86-3.63 (m, 1H), 3.58-3.40 (m, 6H).

Preparation of 21: To a solution of 20 (3.0 g, 11.5 mmol) in pyridine (30.0 mL) was added DMTrCl (3.9 g, 11.5 mmol), then the reaction mixture was stirred at r.t. for 2 h, LC-MS showed that the raw material was 20.0% and The ratio of product to by-product was 3:1, then the mixture was poured into a solution of NaHCO₃ (100.0 mL), and extracted with EA(100.0 mL), washed with brine and dried over Na₂SO₄, filtered and concentrated to get residue, which was purified by silica gel column to give The purified products and by-products were 5.0 g in total, then the product was purified by SFC to give 21 (1.8 g,). ESI-LCMS: m/z=561.2 [M+H]⁺; ¹H NMR (400 MHz, DMSO-d6) δ 11.31 (s, 1H), 7.69 (d, J=8.1 Hz, 1H), 7.45-7.15 (m, 8H), 6.88 (d, J=8.5 Hz, 4H), 5.71 (d, J=6.8 Hz, 1H), 5.64 (d, J=8.0 Hz, 1H), 4.79 (t, J=5.5 Hz, 1H), 3.74 (s, 6H), 3.60 (s, 1H), 3.51 (d, J=5.5 Hz, 3H), 3.11 (d, J=6.7 Hz, 1H), 3.02 (d, J=7.0 Hz, 1H).

Preparation of 22: To a solution of 21 (1.8 g, 3.2 mmol) in DCM (20.0 mL) was added CEP[N(iPr)2]2 (1.0 g, 3.4 mmol) and DCI (321.0 mg, 2.7 mmol). The mixture was stirred at r.t. for 1 h. LC-MS showed 21 was consumed completely. The solution was washed with solution of NaHCO₃ twice and washed with brine and dried over Na₂SO₄. Then concentrated to give a residue, which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=I/O within 20.0 min, the eluted product was collected at CH₃CN/H₂O (0.5% NH₄HCO₃)=90/10; Detector, UV 254 nm. This resulted in to give 22(2.0 g, 82% yield). ESI-LCMS: m/z=761.2 [M+H]; ¹H NMR (400 MHz, DMSO-d6) δ 11.35 (s, 1H), 7.73 (dd, J=8.0, 2.0 Hz, 1H), 7.39 (d, J=7.4 Hz, 2H), 7.35-7.18 (m, 7H), 6.94-6.82 (m, 4H), 5.81-5.74 (m, 1H), 5.67 (d, J=8.0 Hz, 1H), 4.11-3.85 (m, 1H), 3.82-3.67 (m, 11H), 3.67-3.50 (m, 5H), 3.17-3.09 (m, 1H), 3.09-3.01 (m, 1H), 2.74 (td, J=5.8, 2.9 Hz, 2H), 1.13 (dd, J=9.2, 6.7 Hz, 13H); ³¹P NMR (DMSO-d6) δ 148.09 (d, J=41.8 Hz).

### Example 9

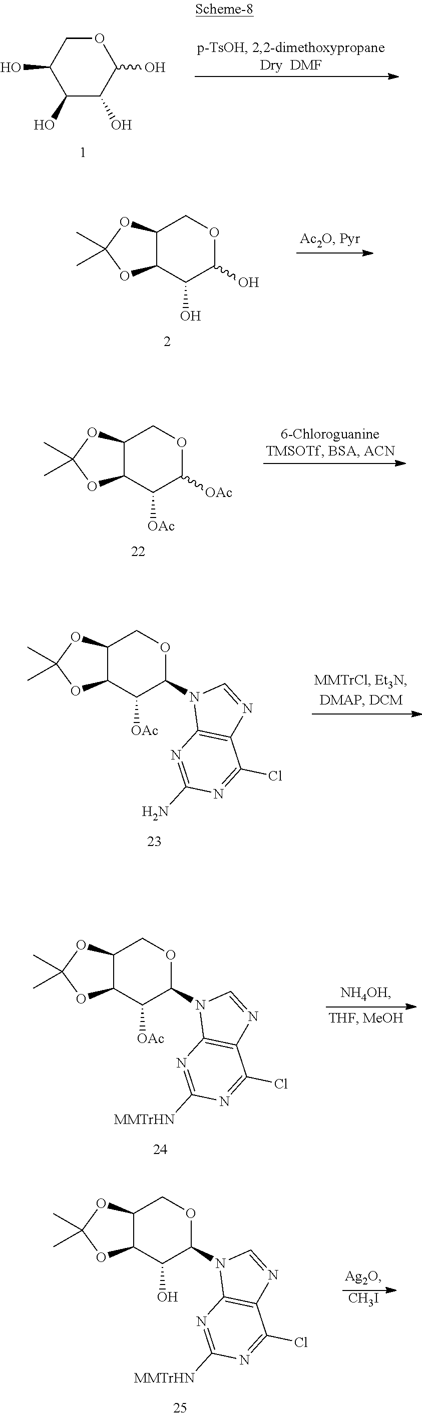

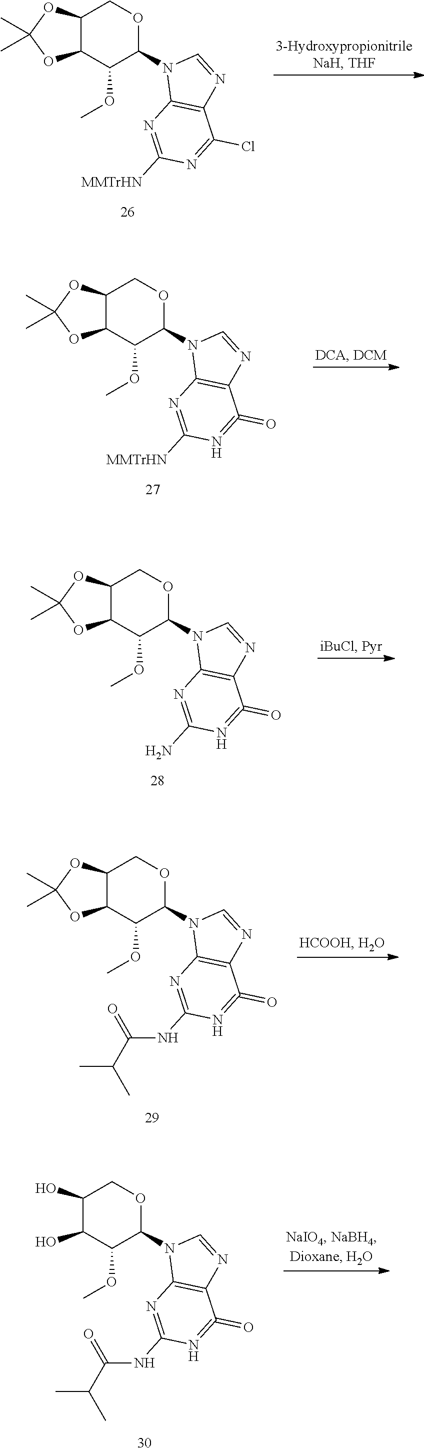

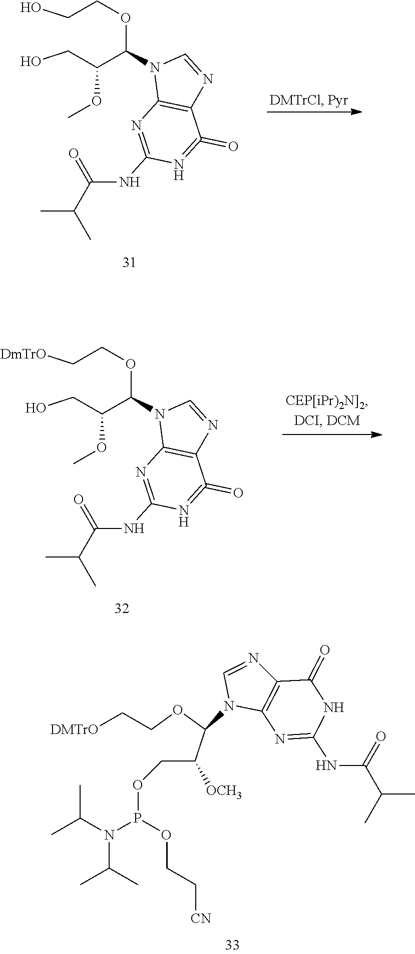

Preparation of 2 (J. Chem. Soc., Perkin Trans. 1, 1992, 1943-1952): To a solution of 1 (150.0 g, 999.1 mmol) in DMF (1000.0 mL) was added P-TsOH (1.7 g, 10.0 mmol), then 2,2-dimethoxy-propane(312.2 g, 3.0 mol) was added to the reaction mixture. The reaction mixture was stirred for 5 h at r.t.. 90.0% 1 was consumed by TLC. Then NaHCO₃ (8.4 g, 99.9 mmol) was added to the reaction mixture, filtered out the solid after 30 min, and concentrated the organic phase by vacuum to obtain crude, which was purified by c.c. (PE:EA=1:1 to 0:1) to get compound 2 (115.0 g, 60.5% yield) as a white solid.

Preparation of 22 (Rajkamal; Pathak, Navendu P.; Halder, Tanmoy; Dhara, Shubhajit; Yadav, Somnath[Chemistry-A European Journal, 2017, vol. 23, #47, p. 11323-11329]): A solution of 2 (115.0 g, 604.6 mmol) in pyridine (600.0 mL) was cooled to 0° C., then Ac₂₀ (185.2 g, 1.81 mol) was added drop wise to the reaction mixture. The reaction was stirred for 2 h at r.t., and the raw material was consumed by TLC. The reaction solution was added into water, extracted product with EA. The organic phase was washed with brine, and dried the organic phase with Na₂SO₄, and concentrated to get 22 (150.0 g, 90.4% yield), which was used for next step directly. ¹H NMR (400 MHz, Chloroform-d) δ 6.20 (d, J=3.4 Hz, 1H), 5.66 (d, J=6.8 Hz, 1H), 5.17 (t, J=6.9 Hz, 1H), 5.10 (dd, J=7.0, 3.4 Hz, 1H), 4.40-4.25 (m, 3H), 4.21 (dd, J=7.0, 6.1 Hz, 1H), 4.16-4.02 (m, 3H), 3.95 (dd, J=12.9, 4.4 Hz, 1H), 2.17 (s, 1H), 2.15-2.03 (m, 12H), 1.56 (d, J=4.0 Hz, 6H), 1.37 (d, J=3.1 Hz, 6H).

Preparation of 23: To a solution of 22 (150.0 g, 546.9 mmol) in ACN (2200.0 mL) was added 6-chloroguanine (139.1 g, 820.4 mmol) and BSA (333.7 g, 1.6 mol) at r.t., then the reaction mixture was replaced with N₂ over 3 times. The reaction was stirred for 30 min at 50° C. After that, the reaction mixture was cooled to 0° C. under N₂. Then TMSOTf (182.1 g, 820.4 mmol) was added into the mixture. After addition, the reaction was stirred for 1.5 h at 70° C. TLC and LC-MS showed the raw material was consumed. Concentrated the most organic solvent by vacuum, then the residual was added to an aqueous solution of NaHCO₃ in ice-water, extracted product with EA (4.0 L), dried the organic phase over Na₂SO₄, and filtered and concentrated to get crude, which was purified by c.c. (DCM to DCM:EA=5:1) to get compound 23 (82.0 g, 35.0% yield,) as a white solid. ESI-LCMS: m/z=384.8 [M+H]⁺; ¹H NMR (400 MHz, DMSO-d6) δ 8.23 (s, 1H), 7.04 (d, J=22.3 Hz, 2H), 5.57 (d, J=9.6 Hz, 1H), 5.40 (dd, J=9.6, 7.3 Hz, 1H), 4.48 (dd, J=7.4, 5.4 Hz, 1H), 4.40-4.30 (m, 2H), 4.11 (dd, J=13.6, 2.4 Hz, 1H), 1.81 (s, 3H), 1.55 (s, 3H), 1.34 (s, 3H).

Preparation of 24: To a solution of 23 (82.0 g, 192.3 mmol) in DCM (1000.0 mL) was added Et₃N (59.4 g, 576.9 mmol) and DMAP (2.4 g, 19.2 mmol) at r.t.. The reaction mixture was replaced with N₂ over 3 times, then MMTrCl (90.9 g, 288.4 mmol) was added into the mixture. The reaction mixture was stirred at r.t. overnight. TLC and LC-MS showed that 92.0% raw material was consumed, and the reaction mixture was added to an aqueous solution of NaHCO₃ in ice-water, then extracted product with EA. Washed the organic phase with brine, and dried the organic phase over Na₂SO₄, then concentrated to get crude, which was purified by c.c. (DCM) to give compound 24 (110.0 g, 86.4% yield) as a white solid. ESI-LCMS: m/z=657.1 [M+H]⁺; ¹H NMR (400 MHz, DMSO-d6) δ 8.21 (s, 1H), 7.37-7.31 (m, 4H), 7.29-7.23 (m, 6H), 7.20-7.15 (m, 2H), 6.86-6.80 (m, 2H), 5.75 (s, 1H), 5.23 (dd, J=9.6, 7.2 Hz, 1H), 4.85 (s, 1H), 4.44-4.16 (m, 3H), 3.71 (s, 4H), 1.70 (s, 3H), 1.49 (s, 3H), 1.31 (s, 3H).

Preparation of 25: To a solution of 24 (110.0 g, 164.3 mmol) in a mixed solvent of THE (500.0 mL) and MeOH (160.0 mL) was added NH₄₀H (330.0 mL). The reaction mixture was stirred overnight at r.t., and the raw material was consumed by TLC and LC-MS. The reaction liquid was added into water, extracted product with EA. Washed the organic phase with brine, then dried the organic phase over Na₂SO₄, then concentrated to get the crude, which was purified by c.c. (PE:EA=10:1-1:2) to give compound 25 (98.0 g, 94.2% yield) as a white solid. ESI-LCMS: m/z=615.1 [M+H]; ¹H NMR (400 MHz, DMSO-d6) δ 8.32 (s, 1H), 7.36 (dt, J=8.2, 1.4 Hz, 4H), 7.31-7.21 (m, 6H), 7.15 (t, J=7.2 Hz, 2H), 6.85-6.76 (m, 2H), 5.57 (d, J=4.6 Hz, 1H), 4.69 (s, 1H), 4.25 (dt, J=5.1, 2.4 Hz, 1H), 4.03 (q, J=7.1 Hz, 4H), 3.70 (s, 3H), 3.62-3.44 (m, 1H), 1.51 (s, 3H), 1.31 (s, 3H).

Preparation of 26 (Ref WO2011/95576, 2011, A1): To a solution of 25 (70.0 g, 114.0 mmol) in CH₃I (350.0 mL) was added Ag₂O (79.2 g, 342.0 mmol) at r.t.. Then the reaction mixture was stirred for 4 h at r.t.. TLC and LC-MS showed that the raw material was consumed. Filtered out the residue with diatomite, and concentrated the filtrate by vacuum to get crude, which was purified by c.c. (PE:EA=10:1-1:1) to get compound 26 (28.0 g, 31.3% yield) as a white solid. ESI-LCMS: m/z=629.1 [M+H]⁺.

Preparation of 27: A solution of 3-hydroxy-propionitrile (15.6 g, 219.7 mmol) in THE (200.0 mL) was cooled to 0° C. The reaction mixture was replaced by N₂ over 3 times. Then NaH (12.4 g, 310.0 mmol, 60.0%) was added to the reaction mixture in turn. The reaction was stirred for 30 min at r.t., and then the reaction was cooled to 0° C. again. A solution of 26 (26.0 g, 33.0 mmol) in THE (150.0 mL) was added drop wise to the reaction mixture. Then the reaction mixture was stirred at r.t. overnight. TLC and LC-MS showed the raw material was consumed. The reaction liquid was added into water, extracted product with EA. The organic phase was washed with brine, and dried over Na₂SO₄, then concentrated to get the crude, which was purified by c.c. (DCM:MeOH=50:1-30:1) to get compound 27 (18.0 g, 88.0% yield) as white solid. ESI-LCMS: m/z=610.7 [M+H]⁺; ¹H NMR (400 MHz, DMSO-d6) δ 10.68 (s, 1H), 7.90 (s, 1H), 7.69 (s, 1H), 7.34-7.15 (m, 12H), 6.92-6.81 (m, 2H), 4.46 (d, J=9.5 Hz, 1H), 4.22 (dt, J=5.5, 2.5 Hz, 1H), 4.07 (t, J=6.4 Hz, 1H), 3.84 (dd, J=13.5, 2.1 Hz, 1H), 3.64-3.54 (m, 1H), 3.36 (dd, J=13.3, 2.8 Hz, 1H), 3.08 (s, 3H), 2.59 (t, J=6.0 Hz, 3H), 1.49 (s, 3H), 1.30 (s, 3H).

Preparation of 28 (.; Beigelman, Leonid; Deval, Jerome; Jin, Zhinan WO2014/209979, 2014, Al,): To a solution of 27 (18.0 g, 29.5 mmol) in DCM (300.0 mL) was added triethylsilane (70.0 mL) and DCA (10.0 mL) at r.t.. Then the reaction mixture was stirred for 6 h at r.t., TLC and LC-MS showed that the raw material was consumed. Concentrated the almost organic solvent by vacuum, then PE (600.0 mL) was added to the reaction mixture. Filtered of the organic phase to get the solid, which was purified by MPLC (MeCN:H₂O=40:60 to 50:50) to get compound 28 (7.5 g, 75.0% yield) as a white solid. ESI-LCMS: m/z=338.3 [M+H]⁺; ¹H NMR (400 MHz, DMSO-d6) δ 10.70 (s, 1H), 8.03 (s, 1H), 6.49 (s, 2H), 5.15 (d, J=9.6 Hz, 1H), 4.28 (d, J=5.1 Hz, 2H), 4.20 (d, J=13.6 Hz, 1H), 3.93 (ddd, J=13.3, 10.6, 3.7 Hz, 2H), 3.26 (s, 3H), 1.59 (s, 3H), 1.33 (s, 3H);

Preparation of 29: A solution of 28 (7.0 g, 20.6 mmol) in Pyr (150.0 mL) was cooled to 0° C. Then the reaction mixture was added i-BuCl (6.6 g, 61.8 mmol) drop wise. The reaction mixture was stirred for 30 min, TLC and LC-MS showed the raw material was consumed. The reaction liquid was added to ice-water, extracted product with EA. The organic phase was washed with brine, and dried over Na₂SO₄, and filtered and concentrated to get the crude, which was purified by c.c. (DCM:MeOH=100:1-30:1) to get compound 29 (5.8 g, 68.6% yield) as a white solid. ESI-LCMS: m/z=409.4 [M+H]⁺; ¹H NMR (400 MHz, DMSO-d6) δ 12.13 (s, 1H), 11.66 (s, 1H), 8.39 (s, 1H), 5.24 (d, J=9.6 Hz, 1H), 4.36-4.23 (m, 3H), 3.99-3.88 (m, 2H), 3.27 (s, 4H), 2.78 (hept, J=6.8 Hz, 1H), 1.61 (s, 3H), 1.35 (s, 3H), 1.12 (d, J=6.8 Hz, 6H).

Preparation of 30: A solution of 29 (5.8 g, 14.1 mmol) was added into a mixed solvent of HCOOH (54.0 mL) and H₂O(6.0 mL) at r.t.. Then reaction mixture was stirred for 1 h at r.t.. TLC and LC-MS showed the raw material was consumed. Concentrated the reaction solution by vacuum at r.t. to get compound 30 (5.2 g, 14.0 mmol, 98.0% yield), which was used for next step directly. ESI-LCMS: m/z=368.4 [M+H]⁺; ¹H NMR (400 MHz, DMSO-d6) δ 12.13 (s, 1H), 11.72 (s, 1H), 8.30 (s, 1H), 8.14 (s, 2H), 5.19 (d, J=9.2 Hz, 1H), 3.93 (t, J=9.2 Hz, 1H), 3.85 (dd, J=12.4, 1.9 Hz, 1H), 3.77 (d, J=3.7 Hz, 1H), 3.69-3.62 (m, 2H), 3.20 (s, 3H), 2.79 (h, J=6.8 Hz, 1H), 1.13 (dd, J=6.9, 1.2 Hz, 6H).

Preparation of 31: To a solution of 30 (5.2 g, 14.0 mmol) in dioxane (90.0 mL) and H₂O (30.0 mL) was added NaIO4 (3.7 g, 15.4 mmol) at r.t.. The reaction mixture was stirred for 3 h at r.t.. LC-MS showed the raw material was consumed, and the reaction solution was cooled to 0° C. Then NaBH₄ (970.0 mg, 25.2 mmol) was added to the reaction mixture, and the raw material was consumed after 3 h by LC-MS. The reaction liquid was quenched with ammonium chloride, and adjusted the pH to 6-7 with 1N HCl, the mixture solution was concentrated to get the crude, which was purified by c.c. (DCM:MeOH=100:1-30:1) to get compound 31 (4.0 g, 68.6% yield) as a white solid. ESI-LCMS: m/z=370.4 [M+H]*; ¹H NMR (400 MHz, DMSO-d6) δ 11.91 (d, J=151.0 Hz, 2H), 8.62-8.51 (m, 1H), 8.18 (s, 1H), 7.44-7.33 (m, 1H), 5.62 (d, J=7.9 Hz, 1H), 4.84 (t, J=5.7 Hz, 1H), 4.65 (d, J=5.2 Hz, 1H), 3.84 (dd, J=7.7, 3.5 Hz, 1H), 3.76 (ddd, J=12.1, 4.7, 2.7 Hz, 1H), 3.60 (ddd, J=12.0, 5.8, 3.6 Hz, 1H), 3.46 (d, J=8.8 Hz, 2H), 3.16 (s, 3H), 2.77 (h, J=6.8 Hz, 1H), 1.12 (dd, J=6.8, 2.4 Hz, 6H);

Preparation of 32: A solution of 31 (4.0 g, 6.4 mmol) was dissolved in pyridine(100.0 mL), and the reaction mixture was replaced by N₂ over 3 times, and then DMTrCl (5.1 g, 8.9 mmol) was added to the reaction mixture at r.t.. Then the reaction was stirred for 30 min, TLC and LC-MS showed raw material was consumed. The reaction liquid was added into ice-water, and extracted product with EA. The organic phase was washed with brine, and dried the organic phase over Na₂SO₄, and concentrated to get crude, which was purified by c.c. (DCM:MeOH=100:1-30:1) and SFC to get compound 32 (2.7 g, 37.1% yield) as a white solid. ESI-LCMS: m/z=672.7 [M+H]; ¹H NMR (400 MHz, DMSO-d6) δ 11.50 (s, 2H), 8.22 (s, 1H), 7.32-7.24 (m, 4H), 7.22-7.12 (m, 5H), 6.84 (dd, J=9.0, 2.4 Hz, 4H), 5.63 (d, J=7.9 Hz, 1H), 4.85 (t, J=5.6 Hz, 1H), 3.95 (dt, J=7.4, 3.3 Hz, 1H), 3.85-3.77 (m, 1H), 3.73 (s, 7H), 3.65-3.57 (m, 1H), 3.43 (ddt, J=9.9, 6.9, 3.4 Hz, 1H), 3.05 (ddd, J=10.0, 6.2, 3.3 Hz, 1H), 2.96 (ddd, J=10.0, 5.6, 3.4 Hz, 1H), 2.78 (p, J=6.8 Hz, 1H), 1.11 (d, J=6.7 Hz, 6H).

Preparation of 33: To a solution of 32 (2.7 g, 2.4 mmol) in DCM (35.0 mL) was added DCI (390.0 mg, 2.0 mmol) at r.t.. Then CEP [N(iPr)2]2 (1.2 g, 2.5 mmol) was added to the reaction mixture, then reaction mixture was stirred for 30 min at r.t.. LC-MS showed raw material was consumed. The reaction liquid was added to an aqueous solution of NaHCO₃ into ice-water, and extracted product with DCM, washed the organic phase with brine, and dried the organic phase over Na₂SO₄, then filtered and concentrated to give a residue, which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=I/O within 20.0 min, the eluted product was collected at CH₃CN/H₂O (0.5% NH₄HCO₃)=100/0; Detector, UV 254 nm. This resulted in to give compound 33 (2.0 g, 56.4% yield) as a white solid. ESI-LCMS: m/z=872.3 [M+H]⁺; 1H NMR (400 MHz, DMSO-d6) δ 11.79 (s, 2H), 8.23 (d, J=1.7 Hz, 1H), 7.35-7.07 (m, 9H), 6.92-6.75 (m, 4H), 5.52 (d, J=8.0 Hz, 1H), 4.21 (s, 1H), 4.10-3.99 (m, 1H), 3.84-3.65 (m, 10H), 3.63-3.52 (m, 2H), 3.45 (ddd, J=10.2, 6.7, 3.6 Hz, 1H), 3.34 (s, 1H), 3.22 (s, 3H), 3.07 (ddd, J=10.2, 6.4, 3.4 Hz, 1H), 2.97 (ddd, J=10.0, 5.6, 3.5 Hz, 1H), 2.78 (dt, J=12.2, 6.4 Hz, 3H), 1.20-1.05 (m, 18H), ³¹P NMR (162 MHz, DMSO-d6) δ 148.20, 147.13.

### Example 10

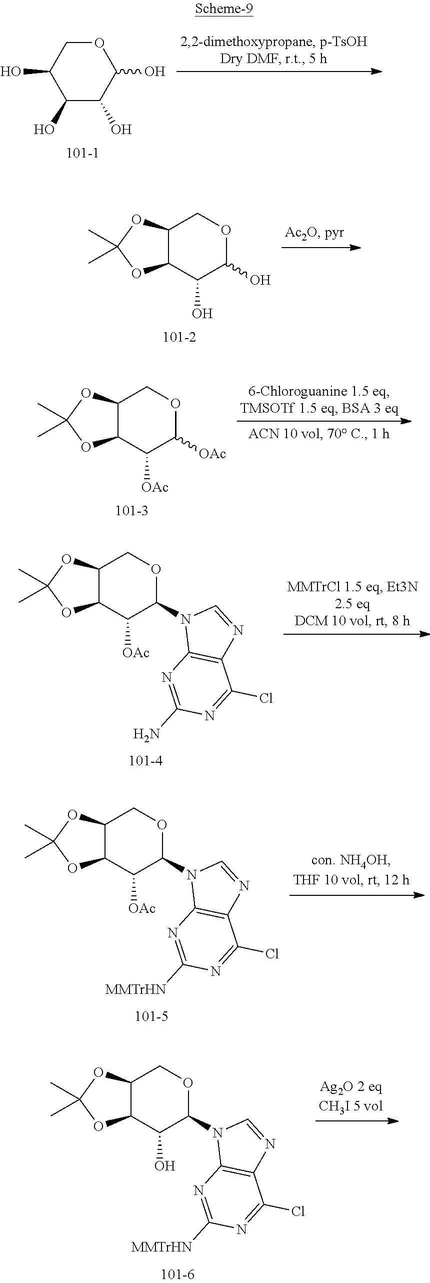

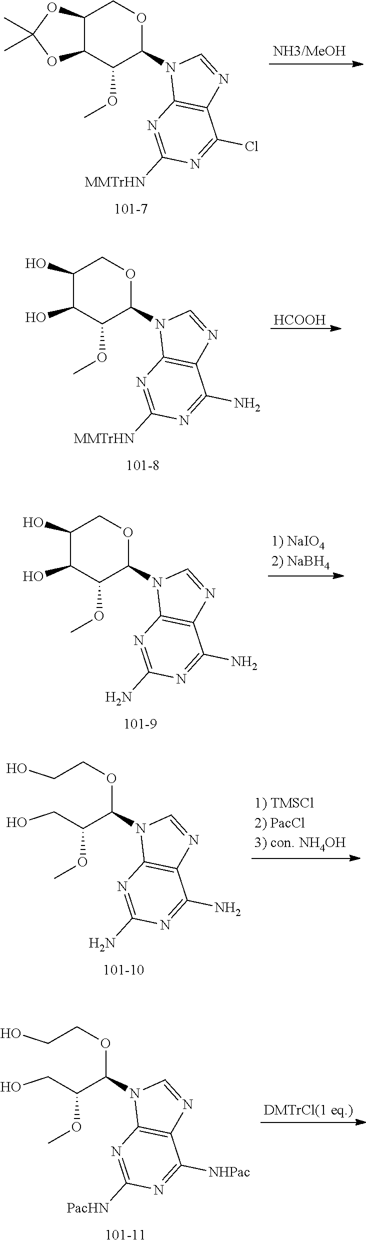

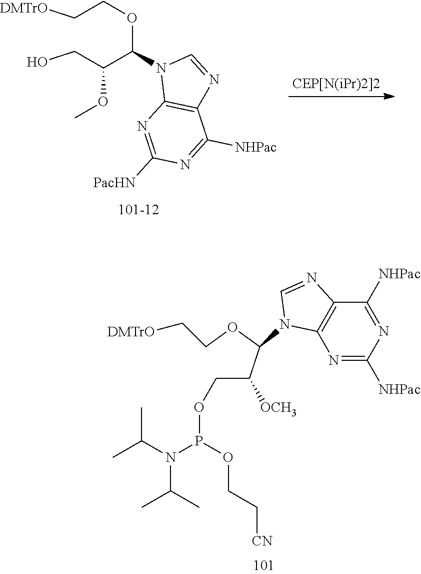

### Example 11

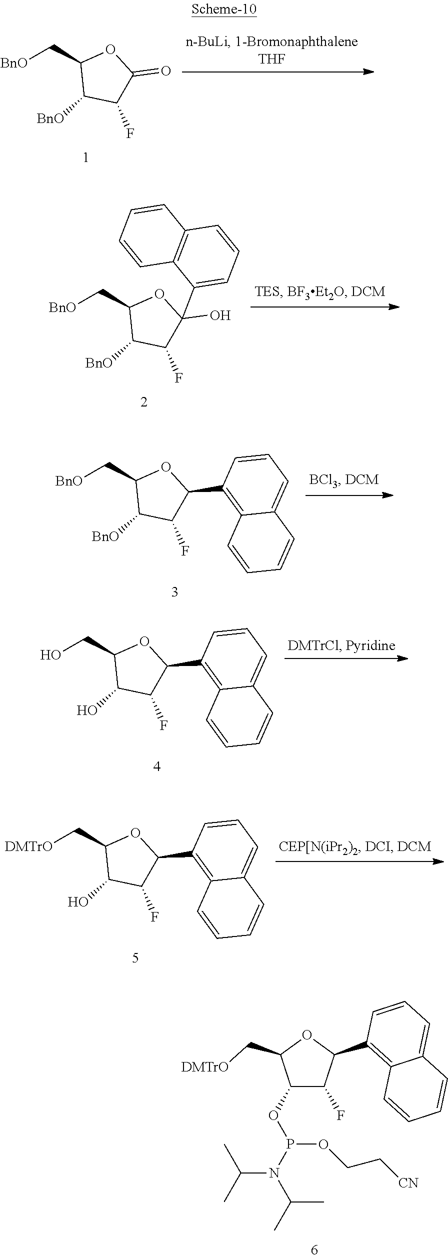

Preparation of 2: To a solution of 1-bromonaphthalene (5.2 g, 25.0 mmol) in dry THE (100.0 mL) was added n-BuLi (13.5 mL, 21.7 mmol, 1.6 M) drop wise at −78° C., then the mixture was stirred at −78° C. for 0.5 h, after that, a solution of 1 (5.5 g, 16.7 mmol) in THE (20.0 mL) was added into the mixture drop wise maintaining inner temperature below −70° C., then the reaction mixture was stirred for 1 h at −70° C. LC-MS showed 1 was consumed completely, the reaction was quenched with saturated ammonium chloride solution(80.0 mL) and extracted with EA, The organic layer was washed with brine, dried over Na₂SO₄, and concentrated under reduced pressure to give a residue, which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=4/1 within 25 min, the eluted product was collected at CH₃CN/H₂O (0.5% NH₄HCO₃)=3/2; Detector, UV 254 nm. This resulted in to give 2 (5.8 g, 76.3% yield) as a white solid. ESI-LCMS: m/z 441 [M−OH]⁻.

Preparation of 3: To the solution of 2 (5.8 g, 12.6 mmol) in DCM (100.0 mL) was added TES (1.7 g, 14.7 mmol) at −78° C., BF₃. Et₂₀ (2.7 g, 18.9 mmol) was added into the mixture drop-wise at −78° C. The mixture was stirred at −40° C. for 1 h. LC-MS showed 2 was consumed completely, the solution was added into a saturated sodium bicarbonate solution (50.0 mL) and extracted with DCM. The organic layer was washed with brine, dried over Na₂SO₄, and concentrated under reduced pressure to give a residue, which was purified by Flash-Prep-HPLC with the following conditions (IntelFlash-1): Column, C18 silica gel; mobile phase, CH₃CN/H₂O (0.5% NH₄HCO₃)=2/3 increasing to CH₃CN/H₂O (0.5% NH₄HCO₃)=4/1 within 25 min, the eluted product was collected at CH₃CN/H₂O (0.5% NH₄HCO₃)=7/3; Detector, UV 254 nm. This resulted in to give 3 (2.7 g, 48.2%) as a white solid. ESI-LCMS: m/z 460 [M+H₂O]; ¹H-NMR (600 MHz, CDCl₃): δ 8.01-8.00 (d, J=6.5 Hz, 1H), 7.88-7.87 (d, J=7.6 Hz, 2H), 7.77-7.76 (d, J=8.2 Hz, 1H), 7.56-7.49 (m, 2H), 7.38-7.23 (m, 11H), 6.98-5.94 (d, J=26.9 Hz, 1H), 5.09-4.99 (dd, J=61.1 Hz, 1H), 4.71-4.69 (d, J=11.6 Hz, 1H), 4.66-4.59 (m, 2H), 4.43-4.41 (d, J=11.6 Hz, 2H), 4.14-4.08 (m, 1H), 4.02-4.00 (dd, J=13.4 Hz, 1H), 3.81-3.78 (dd, J=14.8 Hz, 1H); ¹⁹F-NMR (CDCl₃): δ −193.24.

Preparation of 4: To a solution of 3 (2.7 g, 6.0 mmol) in dry DCM (40.0 mL) was added BCl₃ (36.0 mL, 36.0 mmol, 1 M) drop wise at −78° C., and the reaction mixture was stirred at −78° C. for 0.5 h. LC-MS showed 3 was consumed completely. After completion of reaction, the resulting mixture was quenched with MeOH (20.0 mL), then neutralized with sodium hydroxide solution (40.0 mL, 2 M). The mixture was extracted with DCM and concentrated to give a crude, the crude was dissolved in MeOH (30.0 mL) and added a sodium hydroxide solution (30.0 mL, 4 M), and the mixture was stirred at r.t. for 30 min. The mixture was extracted with EA, the organic layer was washed with brine, dried over Na₂SO₄, and concentrated under reduced pressure to give a residue, which was purified by silica gel column chromatography (DCM:MeOH=40:1-15:1) to give 4 (1.3 g, 81.2%) as a white solid. ESI-LCMS: m/z 261 [M−H]⁻; ¹H-NMR (DMSO-d6): δ 7.98-7.97 (d, J=10.2 Hz, 2H), 7.89-7.87 (m, 2H), 7.63-7.49 (m, 3H), 5.80-5.76 (d, J=26.3 Hz, 1H), 5.43 (s, 1H), 5.00 (s, 1H), 4.85-4.76 (d, J=58.4 Hz, 1H), 4.03-3.85 (m, 3H), 3.68-3.66 (m, 1H), 3.65-3.53 (m, 1H); ¹⁹F-NMR (DMSO-d6): δ −192.76.

Preparation of 5: To a solution of 4 (1.3 g, 5.0 mmol) in pyridine (20.0 mL) was added DMTrCl (6.1 g, 16.0 mmol) at r.t.. The reaction mixture was stirred at r.t. for 1 h. The LC-MS showed 4 was consumed and water (100.0 mL) was added. The product was extracted with EA and the organic layer was washed with brine and dried over Na₂SO₄, concentrated to give the crude, which was further purified by silica gel (EA:PE=1:30-1:10) to give 5 (2.2 g, 78.5%) as a yellow solid. ESI-LCMS: m/z 563 [M−H]; ¹H-NMR (600 MHz, DMSO-d6): δ 8.03-7.99 (m, 2H), 7.91-7.86 (m, 2H), 7.64-7.57 (m, 2H), 7.49-7.48 (d, J=6.8 Hz, 2H), 7.40-7.24 (m, 8H), 6.89-6.88 (m, 4H), 5.92-5.88 (d, J=26.6 Hz, 1H), 5.50-5.49 (d, J=4.5 Hz, 1H), 4.96-4.87 (d, J=56.2 Hz, 1H), 4.18-4.14 (m, 2H), 3.74 (s, 6H), 3.42-3.40 (d, J=9.9 Hz, 1H), 3.33 (m, 2H); ¹⁹F-NMR (DMSO-d6): δ −192.18.

Preparation of 6: To a suspension of 5 (2.2 g, 3.9 mmol) in DCM (20.0 mL) was added DCI (391.0 mg, 3.3 mmol) and CEP[N(iPr)2]2 (1.4 g, 4.7 mmol). The mixture was stirred at r.t. for 1 h. The LC-MS showed 5 was consumed completely. The solution was washed with a saturated sodium bicarbonate solution and brine successively, dried over Na₂SO₄, concentrated to give the crude, which was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=I/O within 20 min, the eluted product was collected at CH₃CN/H₂O (0.5% NH₄HCO₃)=I/O; Detector, UV 254 nm. This resulted in to give 6 (2.5 g, 83.8%) as a white solid. ESI-LCMS: m/z 765 [M+H]; ¹H-NMR (400 MHz, DMSO-d6): δ 8.07-7.86 (m, 4H), 7.64-7.56 (m, 2H), 7.49-7.45 (m, 2H), 7.41-7.21 (m, 8H), 6.89-6.84 (m, 4H), 6.02-5.93 (m, 1H), 5.19-4.98 (m, 1H), 4.61-4.34 (m, 1H), 4.26-4.24 (m, 1H), 3.74-3.73 (m, 6H), 3.70-3.61 (m, 1H), 3.57-3.42 (m, 4H), 3.29-3.24 (m, 1H), 2.67-2.64 (m, 1H), 2.56-2.52 (m, 1H), 1.09-1.04 (m, 1H), 0.98-0.97 (d, J=6.7 Hz, 3H), 0.89-0.87 (d, J=6.7 Hz, 3H); ¹⁹F-NMR (DMSO-d6): δ −191.75, −191.76, −191.84, −191.85; ³¹P-NMR (DMSO-d6): δ 149.51, 149.47, 149.16, 149.14.

### Example 12

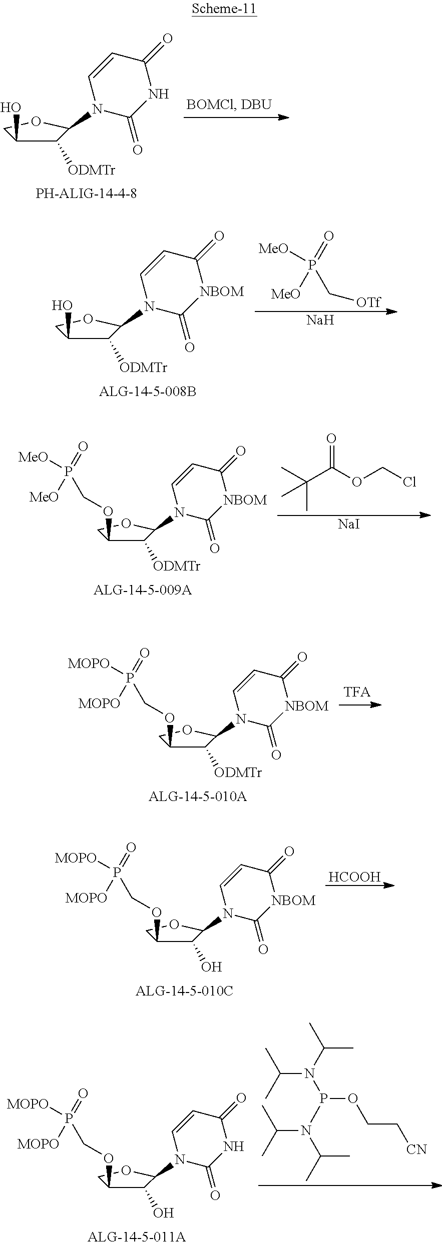

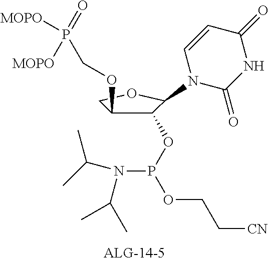

### Preparation of ALG-14-5-008B

To a solution of PH-ALIG-14-4-8 (from Example 5) (6.6 g, 10.86 mmol, 85% purity, 1 eq) and DBU (3.31 g, 21.72 mmol, 3.27 mL, 2 eq) in DMF (70 mL) was added BOMCl (2.55 g, 16.29 mmol, 2.26 mL, 1.5 eq) at 0° C. The mixture was stirred at 20° C. for 12 h. The mixture was diluted with EtOAc (180 mL) and washed with H₂O (80 mL*3), and brine (80 mL), dried over Na₂SO₄, filtered and concentrated under reduced pressure to give a residue. The residue was purified by flash silica gel chromatography (ISCO®; 80 g SepaFlash® Silica Flash Column, Eluent of 10-60%, EtOAc/PE gradient @60 mL/min) to give ALG-14-5-008B (5.2 g, 70% yield,) as a white foam. LCMS (ESI): m/z 659.1.; ¹H NMR (400 MHz, DMSO-d6) δ=7.63 (d, J=8.3 Hz, 1H), 7.40-7.15 (m, 14H), 6.85 (t, J=8.0 Hz, 4H), 5.97 (s, 1H), 5.75 (d, J=8.0 Hz, 1H), 5.39-5.26 (m, 2H), 5.24 (d, J=2.0 Hz, 1H), 4.61 (s, 2H), 3.97 (s, 1H), 3.94-3.83 (m, 2H), 3.68 (d, J=10.0 Hz, 6H), 3.38 (s, 1H)

### Preparation of ALG-14-5-009A

To a solution of ALG-14-5-008B (5.2 g, 8.17 mmol, 1 eq) and dimethoxyphosphorylmethyl trifluoromethanesulfonate (6.67 g, 24.50 mmol, 3 eq) in THE (50 mL) was added NaH (816.65 mg, 20.42 mmol, 60% purity, 2.5 eq) at −5° C. The mixture was stirred at 0° C. for 0.5 h. The reaction mixture was quenched by addition H₂O (50 mL) and diluted with EtOAc (100 mL), then washed with H₂O (50 mL), brine (50 mL), the organic layer was dried over Na₂SO₄, filtered and concentrated under reduced pressure. The residue was purified by flash silica gel chromatography (ISCO®; 80 g SepaFlash® Silica Flash Column, Eluent of 0-50%, EtOAc/DCM gradient @60 mL/min) to give ALG-14-5-009A (4.2 g, 66.42% yield,) as a white foam. LCMS (ESI): m/z 781.1 [M+Na]f, ¹H NMR (400 MHz, CDCl₃) δ=7.49-7.25 (m, 14H), 7.21-7.15 (m, 1H), 6.82 (d, J=8.8 Hz, 4H), 6.46 (s, 1H), 5.65 (d, J=8.2 Hz, 1H), 5.57-5.39 (m, 2H), 4.72 (s, 2H), 4.16-4.07 (m, 2H), 3.93 (dd, J=2.6, 10.8 Hz, 1H), 3.81-3.59 (m, 11H), 3.81-3.59 (m, 1H), 3.24 (dd, J=10.6, 13.5 Hz, 1H), 3.10 (dd, J=9.8, 13.3 Hz, 1H), 2.79 (d, J=2.2 Hz, 1H)³¹p NMR (CD₃CN) 6=22.37 (s)

### Preparation of ALG-14-5-010A

To a solution of ALG-14-5-009A (4.6 g, 6.06 mmol, 1 eq) and NaI (2.73 g, 18.19 mmol, 3 eq) in MeCN (15 mL) was added chloromethyl 2,2-dimethylpropanoate (3.65 g, 24.25 mmol, 3.51 mL, 4 eq). The mixture was stirred at 85° C. for 24 h. The reaction mixture was concentrated under reduced pressure to give a residue. The residue was purified by flash silica gel chromatography (ISCO®; 40 g SepaFlash® Silica Flash Column, Eluent of 0-50%, EtOAc/PE gradient @40 mL/min) to give ALG-14-5-010A (2.7 g, 44.6% yield) as a pale yellow solid. LCMS (m/z): 981.1 [M+Na]+.

### Preparation of ALG-14-5-010C

To a solution of ALG-14-5-010A (2.7 g, 2.82 mmol, 1 eq) in DCM (20 mL) was added Et₃SiH (645.45 mg, 2.82 mmol, 5 mL, 1 eq), followed by addition of TFA (1.54 g, 13.51 mmol, 1 mL, 4.80 eq). The mixture was stirred at 20° C. for 0.5 h. The reaction mixture was concentrated under reduced pressure to give a residue. The residue was purified by flash silica gel chromatography (ISCO®; 24 g SepaFlash® Silica Flash Column, Eluent of 0-50%, EtOAc/DCM gradient @30 mL/min) to give ALG-14-5-010C (1.6 g, 84.82% yield,) as a pale yellow solid. LCMS (ESI): m/z 679.1 [M+Na]+; ¹H NMR (400 MHz, CDCl₃) δ=7.44 (d, J=8.2 Hz, 1H), 7.38-7.26 (m, 5H), 5.76 (d, J=8.2 Hz, 1H), 5.69-5.62 (m, 4H), 5.51-5.43 (m, 1H), 5.51-5.43 (m, 1H), 4.70 (s, 2H), 4.30 (s, 1H), 4.26-4.06 (m, 4H), 3.90 (dd, J=4.9, 8.4 Hz, 2H), 3.22-3.06 (m, 1H), 1.22 (s, 18H)³¹P NMR (162 MHz, CD₃CN) 6=20.25 (s, 1P).

### Preparation of ALG-14-5-011A

To a mixture of ALG-14-5-010C (1.4 g, 2.13 mmol, 1 eq) in isopropanol (20 ml) and H₂O (2 mL) added Pd/C (1.4 g,) and HCOOH (51.22 mg, 1.07 mmol, 2 mL) under N₂. The suspension was degassed under vacuum and purged with H₂ several times. The mixture was stirred under H₂ (15 PSI) at 15° C. for 5 h. The reaction mixture was filtered and the filtrate was concentrated to give a residue. The residue was purified by flash silica gel chromatography (ISCO®; 24 g SepaFlash® Silica Flash Column, Eluent of 0-50%, EtOAc/DCM gradient @30 mL/min) to give ALG-14-5-011A (848 mg, 74.14% yield) as a white foam. LCMS (ESI): m/z 537.0 [M+H]^{+; 1}H NMR (400 MHz, CDCl₃) δ=10.01 (s, 1H), 7.53 (d, J=8.0 Hz, 1H), 5.78-5.63 (m, 6H), 4.40 (s, 1H), 4.35-4.22 (m, 3H), 4.11 (d, J=1.5 Hz, 1H), 3.88 (d, J=8.5 Hz, 2H), 1.22 (s, 18H)³¹P NMR (162 MHz, CD₃CN) 6=20.17 (s, 1P.)

### Preparation of ALG-14-5

To a solution of ALG-14-5-011A (848 mg, 1.58 mmol, 1 eq) in DCM (10 mL) was added 3-bis(diisopropylamino)phosphanyloxypropanenitrile (571.73 mg, 1.90 mmol, 602.45 uL, 1.2 eq) at 0° C., followed by addition of 1H-imidazole-4,5-dicarbonitrile (186.7 mg, 1.58 mmol, 1 eq). The mixture was stirred at 15° C. for 1 h. The reaction mixture was quenched by addition of sat. aq. NaHCO₃ (10 mL) and diluted with DCM (20 mL). Then the organic layer was washed with sat. aq. NaHCO₃ (10 mL*2), dried over Na₂SO₄, filtered and concentrated under reduced pressure. The residue was purified by flash silica gel chromatography (ISCO®; 12 g SepaFlash® Silica Flash Column, Eluent of 0-50%, phase A: PE with 0.5% TEA; phase B: EA with 10% EtOH, 30 mL/min) to give ALG-14-5 (720 mg, 61.21% yield,) as a colorless oil. LCMS (ESI): m/z 737.1 [M+H]⁺; ¹H NMR (400 MHz, CD₃CN) 6=9.17 (s, 1H), 7.49 (d, J=8.0 Hz, 1H), 5.91-5.77 (m, 1H), 5.65-5.54 (m, 5H), 4.49-4.26 (m, 2H), 4.23-4.07 (m, 2H), 3.92-3.55 (m, 6H), 2.71-2.61 (m, 2H), 1.24-1.16 (m, 30H)³¹P NMR (162 MHz, CD₃CN) δ=151.59.

### Example 13: Synthesis of 102

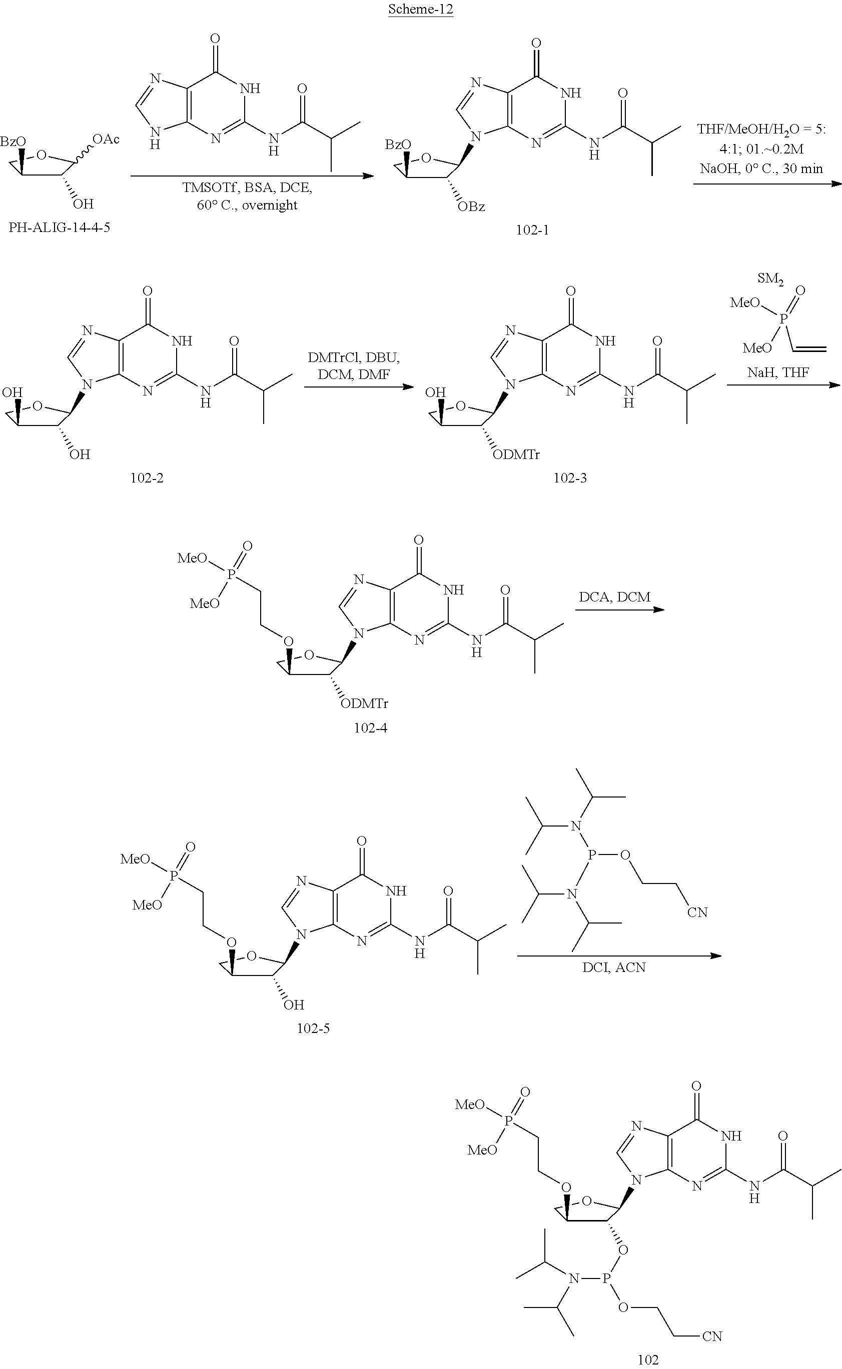

### Example 14: Synthesis of 103

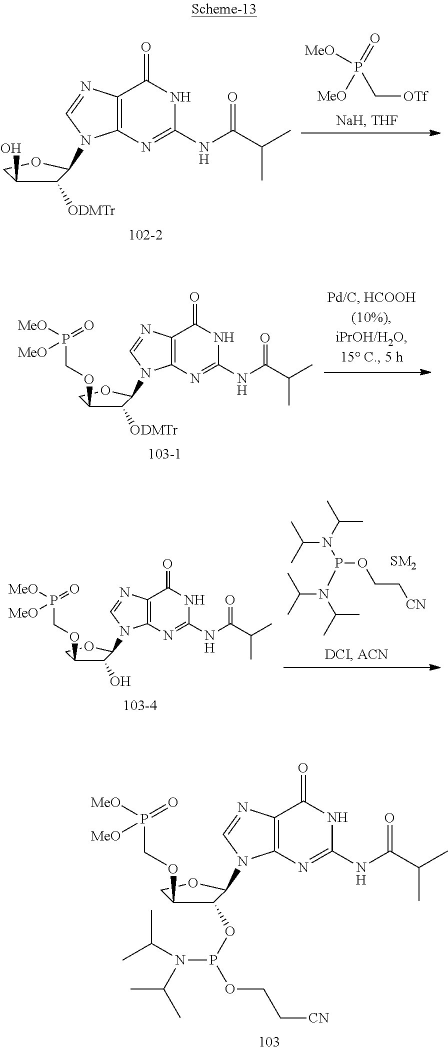

### Example 15: Synthesis of 104

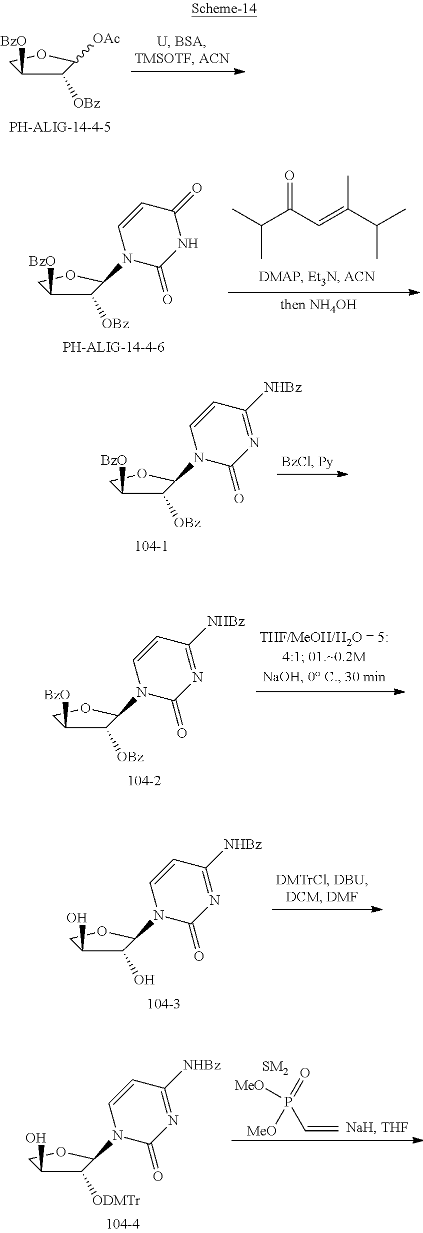

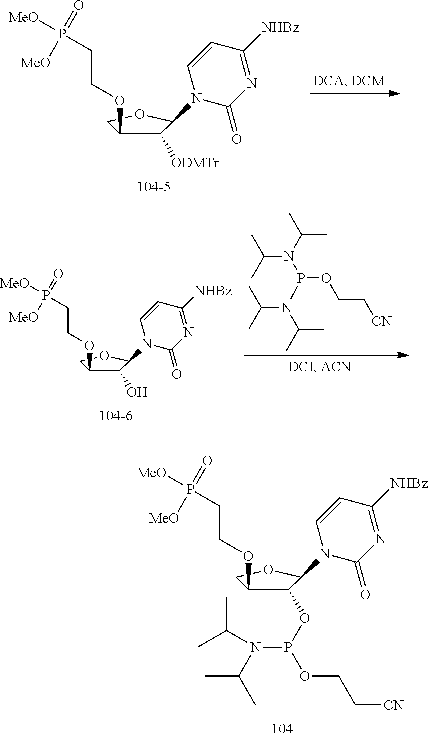

### Example 16: Synthesis of 105

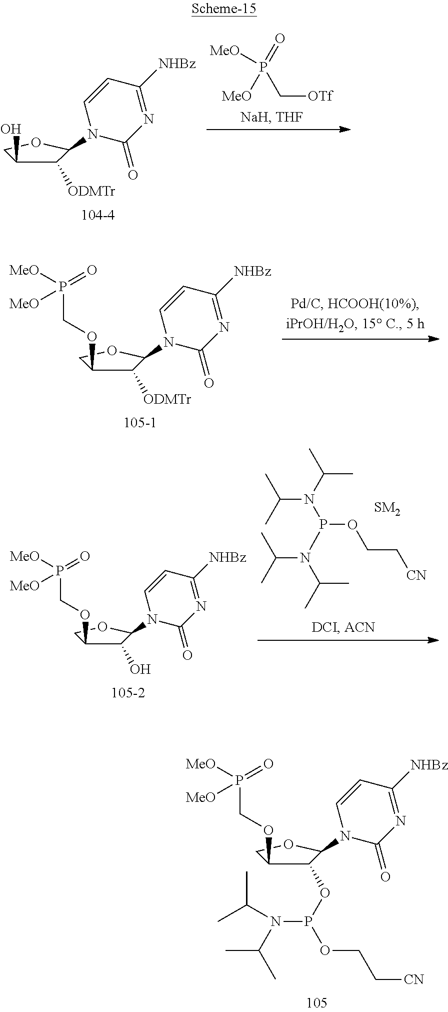

### Example 17

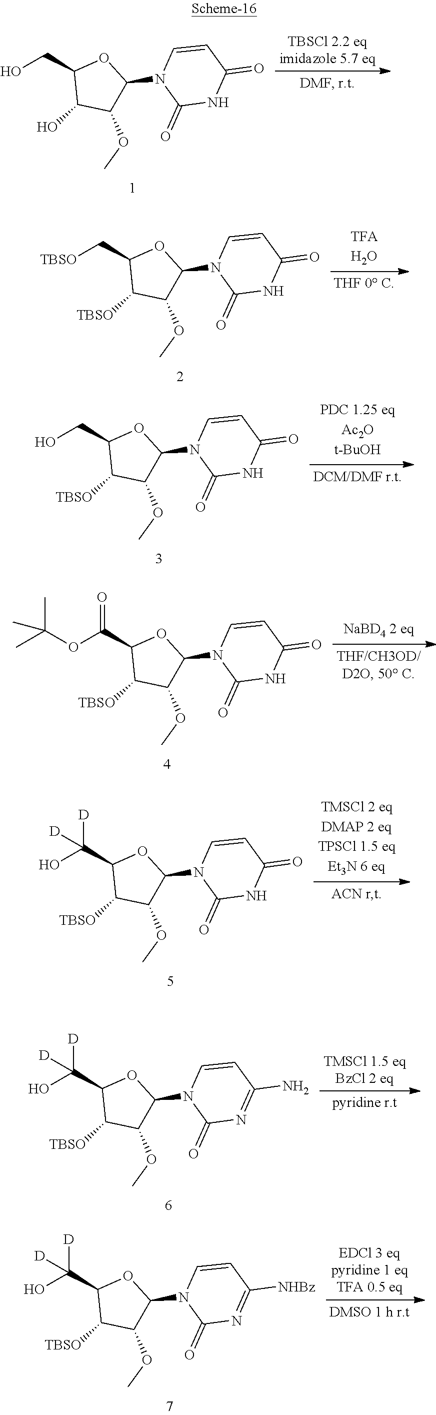

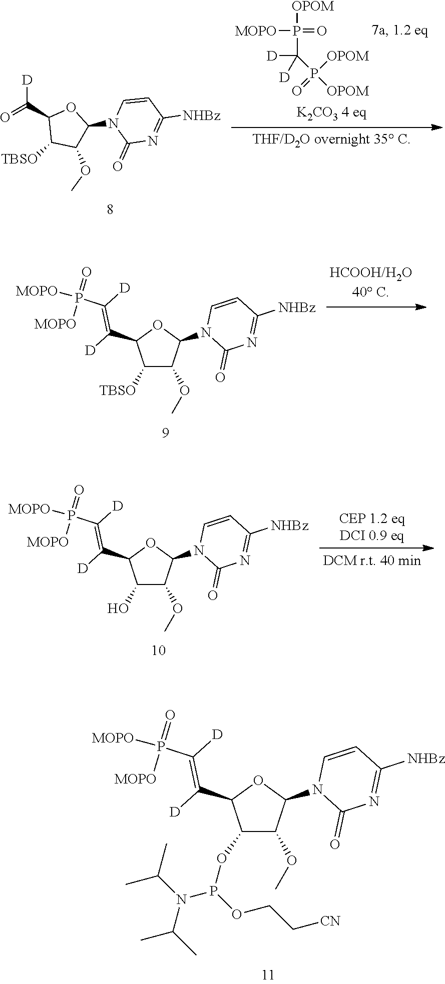

Preparation of 2: A 2 L three-necked round bottom flask equipped with magnetic stirrer and thermometer was charged with 1 (60.0 g, 228.8 mmol) in dry DMF (600.0 mL) at r.t., imidazole (95.2 g, 1.3 mol) was added into the mixture reaction, then the reaction mixture was cooled down to turn 5° C., TBSCl (76.8 g, 499.3 mmol) was added into the mixture reaction, the reaction mixture was allowed to stir for 12 h at r.t. 1 was consumed by LCMS, then the reaction mixture was added in the saturated sodium bicarbonate solution (1.0 L), after quenching the reaction, the aqueous layer was extracted with EA (400.0 mL*2), the combined organic layer was washed with saturated brine and dried over anhydrous sodium sulfate, the organic layer was concentrated to get crude 2 (110.2 g, 212.8 mmol, 93.1% yield) as a white solid, the crude product was used directly for the next step without purification. ESI-LCMS: m/z=487.3 [M+H]⁺.

Preparation of 3: A 3 L three-necked round bottom flask equipped with magnetic stirrer and thermometer was charged with 2 (117.0 g, 225.9 mmol) in THE (550.0 mL) at r.t., water (275.0 mL) was added into the mixture reaction, then the reaction mixture was cooled down to turn 0° C. and add TFA (275.0 mL) by constant pressure funnel after 4 h, the reaction mixture was allowed to stir for 2 h at 0° C. 2 was consumed by TLC. Then, reaction mixture was added in a mixture solvent of ammonium hydroxide (250.0 mL) and water (800.0 mL) at 0° C., after quenching the reaction, the aqueous layer was extracted with EA(500.0 mL*2), the combined organic layer was washed with saturated brine and dried over anhydrous sodium sulfate, the organic layer was concentrated to get crude which was purified by silica gel column chromatography (PE:EA=10:1 to 0:1) to give compound 3 (51.1 g, 59.3% yield) as a white solid. ¹H-NMR (600 MHz, DMSO-d6): δ=11.35 (s, 1H), 7.919 (d, J=6 Hz, 1H), 5.82 (s, 1H), 5.65 (d, J=6 Hz, 1H), 5.18 (s, 1H), 4.29 (s, 1H), 3.83 (s, 2H), 3.65 (d, J=12 Hz, 1H), 3.53 (d, J=6 Hz, 1H), 3.32 (d, J=6 Hz, 1H), 0.87 (s, 9H), 0.08 (s, 6H). ESI-LCMS: m/z=373.1 [M+H]⁺.

Preparation of 4: A 3 L three-necked round bottom flask equipped with magnetic stirrer and thermometer was charged with 3 (50.0 g, 131.5 mmol) in a mixture solvent of DCM (250.0 mL) and DMF (70.0 mL) at r.t., the mixture solution was cooled down to turn 5° C., PDC (63.1 g, 164.4 mmol) and t-BuOH (200.0 mL) were added into the mixture reaction, keep the reaction at 5° C. and add Ac₂₀ (130.0 mL) by constant pressure funnel after 0.5h, the reaction mixture was allowed to stir for 4 h at r.t.. 3 was consumed by lc-ms, then the reaction mixture was added in the saturated sodium bicarbonate (400.0 mL), after quenching the reaction, the aqueous layer was extracted with DCM (500.0 mL*2), the combined organic layer was washed with saturated brine and dried over anhydrous sodium sulfate, the organic layer was concentrated to get crude which was purified by silica gel column chromatography (PE:EA=10:1 to 2:1) to give compound 4 (29.8 g, 50.6% yield) as a white solid. ¹H-NMR (DMSO d6): 6=11.42 (s, 1H), 8.04 (d, J=6 Hz, 1H), 5.82 (s, 1H), 5.78 (d, J=6 Hz, 1H), 4.44 (s, 1H), 4.25 (s, 1H), 3.84 (s, 1H), 3.32 (s, 3H), 1.46 (s, 9H), 0.89 (s, 9H), 0.12 (s, 6H). ESI-LCMS: m/z=443.1 [M+H]⁺.

Preparation of 5: To a solution of 4 (33.0 g, 74.7 mmol) in dry THE (330.0 mL) was added CH₃₀D (66.0 mL) and D20 (33.0 mL) at r.t. Then the reaction mixture was added NaBD₄ (9.4 g, 224.0 mmol) three times per an hour at 50° C. The solution was stirred at 50° C. for 3 h. LCMS showed 4 was consumed. Water (300.0 mL) was added. The product was extracted with EA (2*300.0 mL). The organic layer was washed with brine and dry over by Na₂SO₄. Then the solution was concentrated under reduced pressure, crude was purified by by silica gel column chromatography (PE:EA=10:1 to 3:1) to give 5 (19.1 g, 68.5% yield) as a white solid. ¹H-NMR (600 MHz, DMSO d6): 6=11.35 (s, 1H), 7.92-7.91 (d, J=6 Hz, 1H), 5.83-5.82 (d, J=6 Hz, 1H), 5.66-5.65 (d, J=6 Hz, 1H), 5.14 (s, 1H), 4.30-4.28 (m, 1H), 3.84-3.82 (m, 2H), 3.34 (s, 3H), 0.88 (s, 9H), 0.09 (s, 6H). ESI-LCMS: m/z 375 [M+H]⁺. Preparation of 6: To a solution of 5 (19.1 g, 51.1 mmol) in dry ACN (190.0 mL) was added Et₃N (20.7 g, 204.6 mmol) at r.t. and TMSCl (11.1 g, 102.1 mmol) at 0° C. Then the reaction mixture was stirred at r.t. for 40 min. LCMS showed 5 was consumed and an intermediate was formed. Then the solution was added DMAP (12.5 g, 102.3 mmol), Et₃N (10.3 g, 102.1 mmol) and TPSCl (23.2 g, 76.6 mmol). The reaction mixture was stirred at r.t. for 15 h. LCMS showed the intermediate was consumed and conformed another intermediate. Then was added NH₄₀H (200.0 mL) and stirred at r.t. for 24 h to give the mixture of product. The product was extracted with EA (2*200.0 mL). The organic layer was washed with brine and dry over by Na₂SO₄. Then the solution was concentrated under reduced pressure, crude was purified by Flash-Prep-HPLC with the following conditions (IntelFlash-1): Column, C18 silica gel; mobile phase, CH₃CN/H₂O=1/2 increasing to CH₃CN/H₂O=I/O within 20 min, the eluted product was collected at CH₃CN/H₂O=I/O; Detector, UV 254 nm. This resulted in to give 6 (14.0 g, 73.7% yield). ¹H-NMR (DMSO-d₆): δ=7.89-7.88 (d, J=6 Hz, 1H), 7.20-7.18 (d, J=12 Hz, 2H), 5.85-5.84 (d, J=6 Hz, 1H), 5.73-5.72 (d, J=6 Hz, 1H), 5.09 (s, 1H), 4.24-4.23 (m, 1H), 3.81-3.80 (d, J=6 Hz, 1H), 3.69-3.68 (m, 1H), 3.36 (s, 3H), 0.87 (s, 9H), 0.07 (s, 6H). ESI-LCMS: m/z 374 [M+H]⁺.

Preparation of 7: To a solution of 6 (14.0 g, 37.5 mmol) in pyridine (140.0 mL) was added TMSCl (6.3 g, 58.0 mmol) at 0° C. and the mixture was stirred at r.t. for 1.5 h. LCMS showed 6 was consumed and an intermediate(a) was formed. Then was added BzCl (10.8 g, 76.8 mmol) at 0° C. and the mixture was stirred at r.t. for 1.5 h. LCMS showed the intermediate was consumed and another intermediate was formed. Then the mixture was added NH₄₀H (30.0 mL) and was stirred at r.t. for 15 h. LCMS showed the intermediate was consumed. Water (300.0 mL) was added. The solution was extracted with EA (2*200.0 mL). The organic layer was washed with brine and dry over by Na₂SO₄. Then the solution was concentrated under reduced pressure, crude was purified by Flash-Prep-HPLC with the following conditions (IntelFlash-1): Column, C18 silica gel; mobile phase, CH₃CN/H₂O=1/1 increasing to CH₃CN/H₂O=I/O within 20 min, the eluted product was collected at CH₃CN/H₂O=I/O; Detector, UV 254 nm. This resulted in to give 7 (10.5 g, 58.6% yield). ¹H-NMR (600 MHz, DMSO d6): 6=11.29 (s, 1H), 8.53-8.52 (d, J=6 Hz, 1H), 8.01-8.00 (d, J=6 Hz, 2H), 7.63-7.61 (m, 1H), 7.52-7.50 (m, 2H), 7.36 (s, 1H), 5.88 (s, 1H), 5.24 (s, 1H), 4.28-4.26 (m, 1H), 3.91 (s, 1H), 3.81-3.79 (m, 1H), 3.46 (s, 3H), 0.87 (s, 9H), 0.08 (s, 6H). ESI-LCMS: m/z 478 [M+H]⁺.

Preparation of 8: To a solution of 7 (10.5 g, 22.0 mmol) in DMSO (105.0 mL) was added EDCl (12.7 g, 66.0 mmol), dry pyridine (1.7 g, 22.0 mmol) at r.t. and TFA (1.3 g, 11.0 mmol) at 0° C. Then the reaction mixture was stirred for 1 h. LCMS showed 7 was consumed. Water (100.0 mL) was added. The solution was extracted with EA (2*200.0 mL). The organic layer was washed with brine and dry over by Na₂SO₄. Then the solution was concentrated under reduced pressure to give the crude product 8 which was used in next step directly. ESI-LCMS: m/z 475 [M+H]⁺.

Preparation of 9: To a solution of 8 in dry THE (120.0 mL) and D20 (40.0 mL) was added K₂CO₃ (12.2 g, 88.1 mmol) and 7a (16.8 g, 26.5 mmol) then the reaction mixture was stirred for 15 h at 35° C. under the N₂ atmosphere. LCMS showed 95% 7 was consumed. Water (60.0 mL) was added. The solution was extracted with EA (2*150.0 mL). The organic layer was washed with brine and dry over by Na₂SO₄. Then the solution was concentrated under reduced pressure, crude was purified by Flash-Prep-HPLC with the following conditions (IntelFlash-1): Column, C18 silica gel; mobile phase, CH₃CN/H₂O=1/1 increasing to CH₃CN/H₂O=I/O within 20 min, the eluted product was collected at CH₃CN/H₂O=4/1; Detector, UV 254 nm. This resulted in to give 9 (9.3 g, 54.1% yield). ¹H-NMR (DMSO-d6) δ=11.33 (s, 1H), 8.17-8.15 (d, J=12, 1H), 8.02-8.00 (d, J=12, 1H), 7.64-7.62 (m, 1H), 7.53-7.50 (m, 2H), 7.44-7.42 (d, J=12, 1H), 4.46-4.44 (d, J=12, 1H), 4.24-4.23 (d, J=6, 1H), 3.93-3.91 (d, J=12, 1H), 1.16 (s, 18H), 0.86 (s, 9H)), 0.08-0.06 (d, J=12, 6H). ESI-LCMS: m/z 782 [M+H]⁺. ³¹P-NMR (DMSO-d6) δ=16.77, 16.00.

Preparation of 10: 9 (9.3 g, 11.9 mmol) in the mixture solution of HOAc (140.0 mL) and H₂O (140.0 mL) was stirred at 30° C. for 15 h. LCMS showed 9 was consumed. The solution was added in the ice water and extracted with EA (2*300.0 mL). The organic layer was quenched to pH=6-7 and then washed with brine and dry over Na₂SO₄. Then the solution was concentrated under reduced, crude was purified by pressure Flash-Prep-HPLC with the following conditions (IntelFlash-1): Column, C18 silica gel; mobile phase, CH₃CN/H₂O=1/1 increasing to CH₃CN/H₂O=I/O within 20 min, the eluted product was collected at CH₃CN/H₂O=2.5/1; Detector, UV 254 nm. This resulted in to give 10 (5.1 g, 64.6% yield). ¹H-NMR (DMSO-d6) δ=9.09 (s, 1H), 7.92-7.85 (m, 3H), 7.60-7.48 (m, 4H), 6.02 (s, 1H), 5.71-5.64 (m, 4H), 4.53-4.51 (m, 1H), 3.94-3.70 (m, 5H), 3.31 (s, 1H), 1.21 (s, 18H). ³¹P-NMR (DMSO-d6) δ=16.45. ESI-LCMS: m/z 668 [M+H]⁺.

Preparation of 11: To a suspension of 10 (4.6 g, 6.9 mmol) in DCM (45.0 mL) added CEOP[N(ipr)₂]₂ (2.5 g, 8.3 mmol), DCI (730.4 mg, 6.2 mmol). The mixture was stirred at r.t. for 1 h. LCMS showed 10 was consumed completely. The solution was quenched by water (40.0 mL), washed with brine (2*20.0 mL) and dry over by Na₂SO₄. Then the solution was concentrated under reduced pressure and the residue was purified by Flash-Prep-HPLC with the following conditions (IntelFlash-1): Column, C18 silica gel; mobile phase, CH₃CN/H₂O=1/1 increasing to CH₃CN/H₂O=I/O within 20 min, the eluted product was collected at CH₃CN/H₂O=4/1; Detector, UV 254 nm. This resulted in to give 11 (4.7 g, 5.4 mmol, 78.3% yield) as a white solid. ¹H-NMR (600 MHz, DMSO-d6) δ=11.34 (s, 1H), 8.18-8.16 (m, 1H), 8.02-8.01 (d, J=6, 2H), 7.65-7.42 (m, 4H), 5.95-5.93 (m, 1H), 5.66-5.61 (m, 4H), 4.64-4.57 (m, 1H), 4.32-4.31 (d, J=6, 1H), 4.12-4.10 (m, 1H), 3.81-3.45 (m, 7H), 2.81-2.79 (m, 2H), 1.16-1.13 (m, 30H). ³¹P-NMR (CDCl₃-d6) δ=150.65, 150.20, 16.64, 15.41. ESI-LCMS: m/z 868 [M+H]⁺;

### Example 18

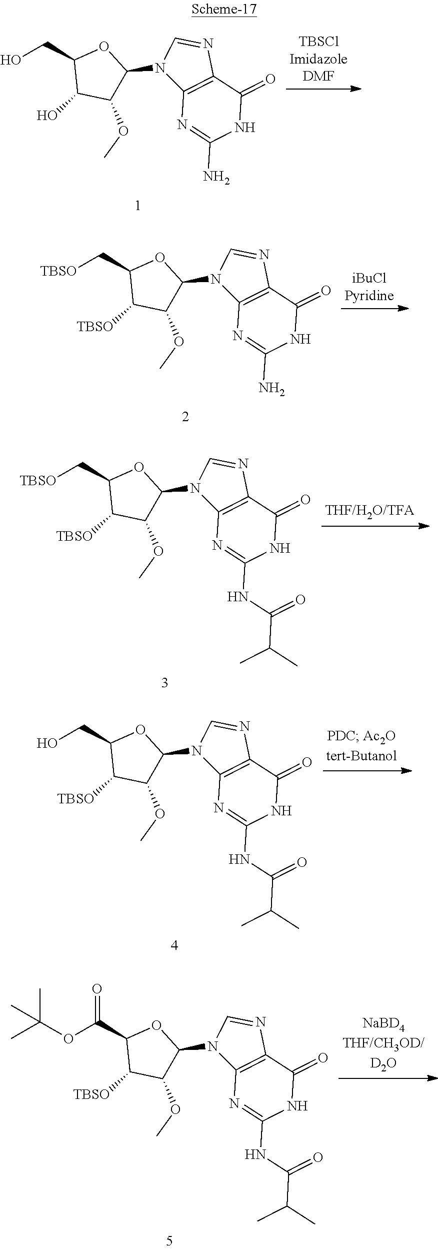 [SMILES: CO[C@@H]1[C@H](O)[C@@H](CO)O[C@H]1n1cnc2c(=O)[nH]c(N)nc21 ; CO[C@@H]1[C@H](O[Si](C)(C)C(C)(C)C)[C@@H](CO[Si](C)(C)C(C)(C)C)O[C@H]1n1cnc2c(=O)[nH]c(N)nc21 ; CO[C@@H]1[C@H](O[Si](C)(C)C(C)(C)C)[C@@H](CO[Si](C)(C)C(C)(C)C)O[C@H]1n1cnc2c(=O)[nH]c(NC(=O)C(C)C)nc21 ; CO[C@@H]1[C@H](O[Si](C)(C)C(C)(C)C)[C@@H](CO)O[C@H]1n1cnc2c(=O)[nH]c(NC(=O)C(C)C)nc21 ; CO[C@@H]1[C@H](O[Si](C)(C)C(C)(C)C)[C@@H](C(=O)OC(C)(C)C)O[C@H]1n1cnc2c(=O)[nH]c(NC(=O)C(C)C)nc21]

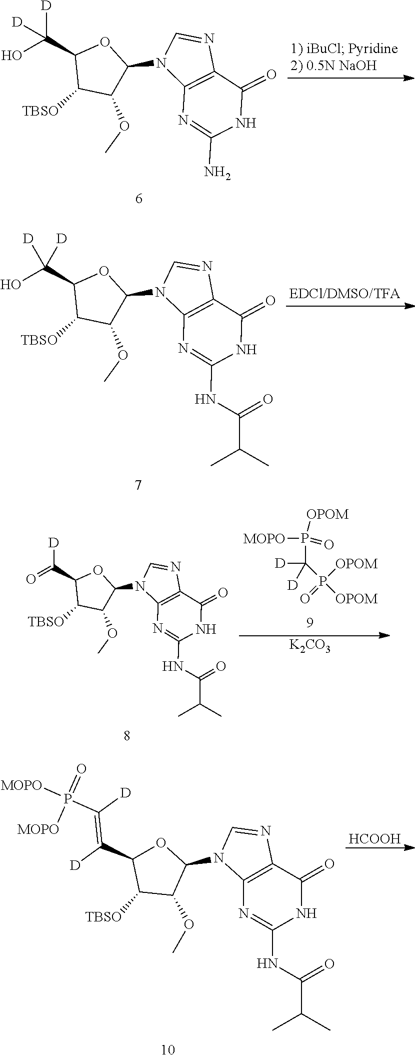

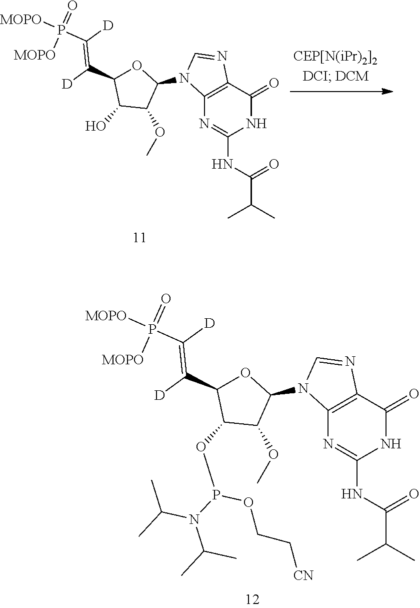

Preparation of 2: 1 (94.5 g, 317.9 mmol) was dissolved in dry DMF (1000 mL) under N₂ atmosphere. To the solution TBSCl (119.3 g, 794.7 mmol) and imidazole (75.8 g, 1.1 mol) was added at 25° C. and stirred for 17 hr. LCMS showed all of 1 consumed. The reaction mixture was washed with H₂O (3000*2 mL), EA (2000*2 mL) and brine (1500 mL). Dried over Na₂SO₄ and concentrated to give crude which goes to the next step. The reaction mixture was concentrated to give crude 2 (200 g, crude). ESI-LCMS: m/z 526 [M+H]⁺.

Preparation of 3: 2 (175.1 g, 333.0 mmol) was evaporated with pyridine and dried in vacuo for two times. The residue was dissolved in pyridine (1500 mL) under N₂. To the solution, i-BuC1 (88.7 g, 832.6 mmol) was added at 5° C. under N₂ atmosphere and stirred for 3 hr. LCMS showed all of 2 consumed. The reaction mixture was washed with H₂O (3000*2 mL), EA (2000*2 mL) and brine (1500 mL). Dried over Na₂SO₄ and concentrated to give crude which goes to the next step. The reaction mixture was concentrated to give crude 3 (228 g, crude). ESI-LCMS: m/z 596 [M+H]⁺.

Preparation of 4: A solution of 3 (225 g, 377.6 mmol) was in THE (2000 mL) was added H₂O (500 mL) and TFA (500 mL) was added at 5° C. Then the reaction mixture was stirred at 5° C. for 1 hr. LCMS showed all of 3 consumed. Con NH₄OH (aq) was added to mixture to quench the reaction until the pH=7-8, then washed with H₂O (2000*2 mL), EA (2000*2 mL) and brine (1500 mL). Dried over Na₂SO₄ and concentrated to give crude which was purified by cc. The reaction mixture was concentrated to give 4 (155.6 g, 83.9% yield). ESI-LCMS: m/z 482 [M+H]⁺.

Preparation of 5: 4 (100 g, 207.6 mmol) was dissolved in dry DMF (1000 mL) under N₂. To the solution, t-BuOH (307.8 g, 4.2 mol), PDC (156.1 g, 0.4 mol) and Ac₂O (212.0 g, 2.1 mol) was added at 25° C. under N₂ atmosphere and stirred at 25° C. for 2 hr. LCMS and TLC showed all of 4 consumed. NaHCO₃(aq) was added to mixture to quench the reaction until the pH=7-8, then washed with H₂O (500*2 mL), EA (500*2 mL) and brine (500 mL). Dried over Na₂SO₄ and concentrated to give crude which was purified by cc. and MPLC. The reaction mixture was concentrated to give 5 (77.3 g, 61.6% yield,). ESI-LCMS: m/z 552 [M−H]+.

Preparation of 6: 5 (40.0 g, 72.6 mmol) was dissolved in dry THE (400 mL) under N₂. To the solution, MeOD (80 mL) and D20 (40 mL) was added at 25° C. under N₂ atmosphere, then NaBD₄ (9.1 g, 217.4 mmol) was added for three times and stirred for 15 hr. LCMS and TLC showed all of 5 consumed. The mixture was concentrated to give crude which goes to the next step. The reaction mixture was concentrated to give crude 6 (30 g, crude). ESI-LCMS: m/z 414 [M+H]⁺

Preparation of 7: 6 (30 g, crude) was evaporated with pyridine and dried in vacuo for two times. The residue was dissolved in dry pyridine (300 mL) under N₂. Then iBuCl (15.5 g, 145.3 mmol) was slowly added to the reaction mixture at 0° C. under N₂ atmosphere and stirred at 25° C. for 1 hr. LCMS and TLC showed all of 6 consumed. NaHCO₃(aq) was added to mixture to quench the reaction until the pH=7.5, then washed with H₂O (1500 mL), EA (1000*2 mL) and brine (1500 mL). Dried over Na₂SO₄ and concentrated to give crude residue R1. NaOH (8 g, 0.2 mol), MeOH (80 mL) and H₂O (20 mL) made up NaOH (aq). The residue R¹ (40 g, 3.63 mmol) was dissolved in pyridine (20 mL). To the solution, 2N NaOH (aq) (100 ml) was added to the solution and stirred the reaction 15 min at 5° C. TLC showed all of R¹ consumed. The mixture was added NH₄C1 to pH=7-8 at 5° C., and concentrated to give crude which was purified by cc. The product was concentrated to give 7 (15.5 g, 33.00% yield over two steps,). ESI-LCMS: m/z 484[M+H]⁺.

Preparation of 8: To a stirred solution of 7 (15.5 g, 32.1 mmol) in DMSO (150 mL) were added EDCl (18.5 g, 96.3 mmol), pyridine (2.5 g, 32.1 mmol), TFA (1.8 g, 16.0 mmol) at room temperature under N₂ atmosphere. The reaction mixture was stirred for 1 h at room temperature. The reaction was quenched with water, extracted with EA (300.0 mL), washed with brine, dried over Na₂SO₄ and evaporated under reduced pressure give a crude 8 (17.3 g, crude) which was used directly to next step. ESI-LCMS: m/z=481 [M+H]⁺.

Preparation of 10: A solution of 8 (17.3 g, crude), 9 (21.4 g, 33.7 mmol) and K₂CO₃ (13.3 g, 96.3 mmol) in dry THE (204 mL) and D20 (34 mL) was stirred 5 h at 40° C. The mixture was quenched with water, extracted with EA (600.0 mL), washed with brine, dried over Na₂SO₄ and evaporated under reduced pressure. The residue was purified by silica gel (PE:EA=5:1˜1:1) to give 10 (9.3 g, 36.6% yield over 2 steps) as a white solid. ESI-LCMS m/z=787[M+H]⁺.

¹H-NMR (DMSO-d6): δ 11.24 (s, 1H, exchanged with D20), 8.74 (d, J=2.7 Hz, 2H), 8.05-8.04 (d, J=7.4 Hz, 2H), 7.65 (t, 1H), 7.57-7.54 (t, 2H), 6.20 (d, J=5.0 Hz, 1H), 5.64-5.58 (m, 4H), 4.77 (t, 1H), 4.70 (t, 1H), 4.57-4.56 (t, 1H), 3.35 (s, 3H), 1.09 (d, J=6.5 Hz, 18H), 0.93 (s, 9H), 0.15 (d, J=1.8 Hz, 6H); ³¹P NMR (DMSO-d6): δ 17.05;

Preparation of 11: To a round-bottom flask was added 10 (9.3 g, 11.5 mmol) in a mixture of H₂O (93 mL) and HCOOH (93 mL). The reaction mixture was stirred for 5 h at 50° C. and 15 h at 35° C. The mixture was extracted with EA (500.0 mL), washed with water, NaHCO₃ solution and brine successively, dried over Na₂SO₄ and evaporated under reduced pressure. The residue was purified by Flash-Prep-HPLC with the following conditions (IntelFlash-1): Column, C18 silica gel; mobile phase, CH₃CN/H₂O (0.5% NH₄HCO₃)=1/2 increasing to CH₃CN/H₂O (0.5% NH₄HCO₃)=I/O within 20 min, the eluted product was collected at CH₃CN/H₂O (0.5% NH₄HCO₃)=3/2; Detector, UV 254 nm. To give product 11 (6.3 g, 78% yield). ¹H-NMR (600 MHz, DMSO-d6): δ 12.17 (s, 1H, exchanged with D20), 11.51 (s, 1H), 8.28 (s, 1H), 6.02-6.03 (d, J=4.2 Hz, 1H), 5.63-5.72 (m, 5H), 4.60 (s, 1H), 4.43-4.45 (m, 2H), 3.40 (s, 1H), 3.38 (s, 1H), 2.83-2.88 (m, 1H), 1.15-1.23 (m, 24H); ³¹P NMR (DMSO-d6) δ=17.69. ESI-LCMS m/z=674 [M+H]⁺.

Preparation of 12: To a solution of 11 (5.6 g, 8.3 mmol) in DCM (55.0 mL) was added the DCI (835 mg, 7.1 mmol), then CEP[N(ipr)2]2 (3.3 g, 10.8 mmol) was added. The mixture was stirred at r.t. for 1 h. The reaction mixture was washed with H₂O (50.0 mL) and brine (50.0 mL), dried over Na₂SO₄ and evaporated under pressure. The residue was purified by Flash-Prep-HPLC with the following conditions (IntelFlash-1): Column, C18 silica gel; mobile phase, CH₃CN/H₂O (0.5% NH₄HCO₃)=1/1 increasing to CH₃CN/H₂O (0.5% NH₄HCO₃)=I/O within 20 min, the eluted product was collected at CH₃CN/H₂O (0.5% NH₄HCO₃)=9/1; Detector, UV 254 nm. The product was concentrated to give 12 (6.3 g, 87% yield) as a white solid. ¹H-NMR (DMSO-d6): δ 12.14 (s, 1H, exchanged with D20), 11.38 (s, 1H), 8.27-8.28 (d, J=6 Hz, 1H), 5.92-5.98 (m, 1H), 5.59-5.65 (m, 4H), 4.57-4.68 (m, 3H), 3.61-3.85 (m, 4H), 3.37 (s, 1H), 3.32 (s, 1H), 2.81-2.85 (m, 3H), 1.09-1.20 (m, 36H); ³¹P NMR (DMSO-d6): δ 150.60, 149.97, 17.59, 17.16; ESI-LCMS m/z=874 [M+H]⁺.

### Example 19: ds-siNA Activity

This example investigates the activity of the ds-siNAs synthesized in Example 1.

Homo sapiens HepG2.2.15 cells were cultured in Dulbecco's Modified Eagle's Medium (DMEM) (ATCC 30-2002) supplemented to also contain 10% fetal calf serum (FCS). Cells were incubated at 37° C. in an atmosphere with 5% CO₂ in a humidified incubator. For transfection of HepG2.2.15 cells with HBV targeting siRNAs, cells were seeded at a density of 15000 cells/well in 96-well regular tissue culture plates. Transfection of cells was carried out using RNAiMAX (Invitrogen/Life Technologies) according to the manufacturer's instructions. Dose-response experiments were done with oligo concentrations of 40, 20, 10, 5, 2.5, 1.25, 0.625, 0.3125, 0.15625 and 0.07813 nM. For each HBV targeting siRNA treatment (e.g., ds-siRNA, as identified by the ds-siNA ID in Table 6), four wells were transfected in parallel, and individual data points were collected from each well. After 24h of incubation with siRNA, media was removed, and cells were lysed and analyzed with a QuantiGene2.0 branched DNA (bDNA) probe set specific for HBV genotype D (also called Hepatitis B virus subtype ayw, complete genome of 3182 base-pairs) as present in cell line HepG2.2.15.

For each well, the HBV on-target mRNA levels were normalized to the GAPDH mRNA level. As shown in Tables 6-10, the activity of the HBV targeting ds-siRNAs was expressed as EC50, 50% reduction of normalized HBV RNA level from no drug control. As shown in Tables 6-10, the cytotoxicity of the HBV targeting ds-siRNAs was expressed by CC50 of 50% reduction of GAPDH mRNA from no drug control.

### Example 20: Use of Ds-siNAs to Treat Hepatitis B Virus Infection

In this example, the ds-siNAs synthesized in Example 1 are used to treat a hepatitis B virus infection in a subject. Generally, a composition comprising a ds-siNA from Tables 1-5 (as identified by the ds-siNA ID) and a pharmaceutically acceptable carrier is administered to the subject suffering from hepatitis B virus. The ds-siNA from Tables 1-5 are conjugated to N-acetylgalactosamine. The ds-siNA is administered at a dose of 0.3 to 5 mg/kg every three weeks by subcutaneous injection or intravenous infusion.

### Example 21: siNA Activity Assays

This example provides exemplary methods for testing the activity of the siNAs disclosed herein.

In vitro Assay:

HepG2.2.15 cells were cultured in Dulbecco's Modified Eagle's Medium (DMEM) (ATCC 30-2002) supplemented to also contain 10% fetal calf serum (FCS). Cells were incubated at 37° C. in an atmosphere with 5% CO₂ in a humidified incubator. For transfection of HepG2.2.15 cells with HBV targeting siRNAs, cells were seeded at a density of 15000 cells/well in 96-well regular tissue culture plates. Transfection of cells was carried out using RNAiMAX (Invitrogen/Life Technologies) according to the manufacturer's instructions. Dose-response experiments were done with oligo concentrations of 40, 20, 10, 5, 2.5, 1.25, 0.625, 0.3125, 0.15625 and 0.07813 nM. For each HBV targeting siRNA treatment (e.g., ds-siRNA, as identified by the ds-siNA ID in Tables 6-10), four wells were transfected in parallel, and individual data points were collected from each well. After 24h of incubation with siRNA, media was removed, and cells were lysed and analyzed with a QuantiGene2.0 branched DNA (bDNA) probe set specific for HBV genotype D (also called Hepatitis B virus subtype ayw, complete genome of 3182 base-pairs) as present in cell line HepG2.2.15.

For each well, the HBV on-target mRNA levels were normalized to the GAPDH mRNA level. As shown in Tables 6-10, the activity of the HBV targeting ds-siRNAs was expressed as EC50, 5000 reduction of normalized HBV RNA level from no drug control. As shown in Tables 6 and 10, the cytotoxicity of the HBV targeting ds-siRNAs was expressed by CC50 of 50% reduction of GAPDH mRNA from no drug control.

TABLE 6
siNA Comprising 2′-Fluoro Nucleotides

| | | | Emax | CC₅₀ |
|---|---|---|---|---|
| Name | SS/AS 5′ to 3′ | EC₅₀ | (%) | (nM) |
| ds-siNA-001 | mGpsmUpsmGmGfUmGfGfAfCmUmUmCmUmCmUmCmAmAmU | A | 71 | >45 |
| | (SEQ ID NO: 1) | | | |
| | mApsf2PpsmUmGmAfGmAmGmAmAmGmUmCfCmAfCmCmAmCpsmGpsmA | | | |
| | (SEQ ID NO: 2) | | | |
| ds-siNA-002 | mGpsmUpsmGmGfUmGfGfAfCmUmUmCmUmCmUmCmAmAmU | B | 59 | >45 |
| | (SEQ ID NO: 3) | | | |
| | mApsfUpsmUmGmAfGmAmGmAmAmGmUmCf4PmAfCmCmAmCpsmGpsmA | | | |
| | (SEQ ID NO: 4) | | | |
| ds-siNA-003 | mGpsmUpsmGmGfUmGfGfAfCmUmUmCmUmCmUmCmAmAmU | A | 68 | >45 |
| | (SEQ ID NO: 5) | | | |
| | mApsfUpsmUmGmAfGmAmGmAmAmGmUmCfCmAf2PmCmAmCpsmGpsmA | | | |
| | (SEQ ID NO: 6) | | | |
| ds-siNA-004 | mGpsmUpsmGmGfUmGfGfAfCmUmUmCmUmCmUmCmAmAmU | B | 61 | >45 |
| | (SEQ ID NO: 7) | | | |
| | mApsfUpsmUmGmAfGmAmGmAmAmGmUmCf2PmAf2PmCmAmCpsmGpsmA | | | |
| | (SEQ ID NO: 8) | | | |
| ds-siNA-005 | mGpsmUpsmGmGfUmGfGfAfCmUmUmCmUmCmUmCmAmAmU | B | 71 | >45 |
| | (SEQ ID NO: 9) | | | |
| | mApsfBpsmUmGmAfGmAmGmAmAmGmUmCfCmAfCmCmAmCpsmGpsmA | | | |
| | (SEQ ID NO: 10) | | | |
| ds-siNA-006 | mGpsmUpsmGmGfUmGfGfAfCmUmUmCmUmCmUmCmAmAmU | B | 70 | >45 |
| | (SEQ ID NO: 11) | | | |
| | mApsfUpsmUmGmAfGmAmGmAmAmGmUmCfBmAfCmCmAmCpsmGpsmA | | | |
| | (SEQ ID NO: 12) | | | |
| ds-siNA-007 | mGpsmUpsmGmGfUmGfGfAfCmUmUmCmUmCmUmCmAmAmU | D | — | >45 |
| | (SEQ ID NO: 13) | | | |
| | mApsfUpsmUmGmAfGmAmGmAmAmGmUmCfBmAfBmCmAmCpsmGpsmA | | | |
| | (SEQ ID NO: 14) | | | |
| ds-siNA-008 | mGpsmUpsmGmGfUmGfGfAfCmUmUmCmUmCmUmCmAmAmU | A | 67 | >45 |
| | (SEQ ID NO: 15) | | | |
| | mApsfUpsmUmGmAfGmAmGmAmAmGmUmCf2PmAfCmCmAmCpsmGpsmA | | | |
| | (SEQ ID NO: 16) | | | |
| CONTROL 1 | mGpsmUpsmGmGfUmGfGfAfCmUmUmCmUmCmUmCmAmAmU | A | 61 | >45 |
| | (SEQ ID NO: 41) | | | |
| | mApsfUpsmUmGmAfGmAmGmAmAmGmUmCf | | | |
| | CmAfCmCmAmCpsmGpsmA | | | |
| | (SEQ ID NO: 42) | | | |

For EC₅₀, A = EC₅₀ ≤ 0.1 nM;
B = 0.1 nM ≤ EC₅₀ < 1 nM;
C = 1 nM < EC₅₀ < 10;
D = EC₅₀ ≥10 nM.
mX = 2′-O-methyl nucleotide;
fX = 2′-fluoro nucleotide;
5dcd3X = nucleotide of Formula 17;
5dfX = nucleotide of Formula 16;
vX= 5′ vinyl phosphonate nucleotide;
d2vX = deuterated 5′
vinyl phosphonate nucleotide;
vmX = 5′ vinyl phosphonate, 2′-methyl nucleotide;
f4P = 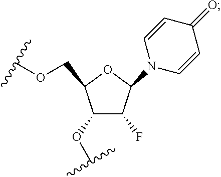 [SMILES: *OC[C@H]1O[C@@H](n2ccc(=O)cc2)[C@H](F)[C@@H]1O*]
f2P = 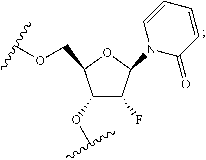 [SMILES: *OC[C@H]1O[C@@H](n2ccccc2=O)[C@H](F)[C@@H]1O*]
fB = 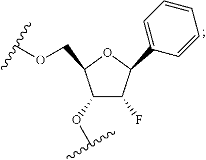 [SMILES: *OC[C@H]1O[C@@H](c2ccccc2)[C@H](F)[C@@H]1O*]
ps = phosphorothioate linkage;
X is a nucleobase (e.g. A, G, C, U or T)

TABLE 7
siNA Comprising Nucleotide Phosphate Mimics

| Name | SS/AS 5′ to 3′ | EC₅₀ |
|---|---|---|
| CONTROL 2 | mCpsmCpsmGmUfGmUfGfCfAmCmUmUmCmGmC | A |
| | mUmUmCmA-p-ps2-GalNAc4 | |
| | (SEQ ID NO: 43) | |
| | mUpsfGpsmAmAfGmCmGfAmAmGmUmGmCfAmC | |
| | mAfCmGmGpsmUpsmC | |
| | (SEQ ID NO: 44) | |
| ds-siNA-009 | mCpsmCpsmGmUfGmUfGfCfAmCmUmUmCmGmC | A |
| | mUmUmCmAp-ps2-GalNAc4 | |
| | (SEQ ID NO: 17) | |
| | omeco- | |
| | d3UpsfGpsmAmAfGmCmGfAmAmGmUmGmCfAm | |
| | CmAfCmGmGpsmUpsmC | |
| | (SEQ ID NO: 18) | |
| ds-siNA-010 | mCpsmCpsmGmUfGmUfGfCfAmCmUmUmCmGmC | A |
| | mUmUmCmAp-ps2-GalNAc4 | |
| | (SEQ ID NO: 19) | |
| | 4hUpsfGpsmAmAfGmCmGfAmAmGmUmGmCfAm | |
| | CmAfCmGmGpsmUpsmC | |
| | (SEQ ID NO: 20) | |
| ds-siNA-011 | mCpsmCpsmGmUfGmUfGfCfAmCmUmUmCmGmC | A |
| | mUmUmCmAp-ps2-GalNAc4 | |
| | (SEQ ID NO: 21) | |
| | d2vd3UpsfGpsmAmAfGmCmGfAmAmGmUmGmCf | |
| | AmCmAfCmGmGpsmUpsmC | |
| | (SEQ ID NO: 22) | |
| ds-siNA-012 | mCpsmCpsmGmUfGmUfGfCfAmCmUmUmCmGmC | A |
| | mUmUmCmA-p-ps2-GalNAc4 | |
| | (SEQ ID NO: 23) | |
| | v- | |
| | munUpsfGpsmAmAfGmCmGfAmAmGmUmGmCfA | |
| | mCmAfCmGmGpsmUpsmC | |
| | (SEQ ID NO: 24) | |
| ds-siNA-013 | mCpsmCpsmGmUfGmUfGfCfAmCmUmUmCmGmC | A |
| | mUmUmCmA-p-ps2-GalNAc4 | |
| | (SEQ ID NO: 25) | |
| | c2o- | |
| | 4hUpsfGpsmAmAfGmCmGfAmAmGmUmGmCfAm | |
| | CmAfCmGmGpsmUpsmC | |
| | (SEQ ID NO: 26) | |
| ds-siNA-014 | mCpsmCpsmGmUfGmUfGfCfAmCmUmUmCmGmC | B |
| | mUmUmCmA-p-ps2-GalNAc4 | |
| | (SEQ ID NO: 27) | |
| | omeco- | |
| | munUpsfGpsmAmAfGmCmGfAmAmGmUmGmCfA | |
| | mCmAfCmGmGpsmUpsmC | |
| | (SEQ ID NO: 28) | |
| ds-siNA-015 | mCpsmCpsmGmUfGmUfGfCfAmCmUmUmCmGmC | A |
| | mUmUmCmA-p-ps2-GalNAc4 | |
| | (SEQ ID NO: 29) | |
| | omeco- | |
| | munUpsfGpsmAmAfGmCmGfAmAmGmUmGmCfA | |
| | mCmAfCmGmGpsmUpsmC | |
| | (SEQ ID NO: 30) | |
| ds-siNA-016 | mGpsmUpsmGmGfUmGfGfAfCmUmUmCmUmCmU | A |
| | mCmAmAmU-p-ps2-GalNAc4 | |
| | (SEQ ID NO: 33) | |
| | d2vmApsfUpsmUmGmAfGmAmGmAmAmGmUmCf | |
| | CmAf2PmCmAmCpsmGpsmA | |
| | (SEQ ID NO: 34) | |
| ds-siNA-023 | 5′- | A |
| | mCpsmCpsmGmUfGmUfGf(4nh)QfAmCmUmUmCm | |
| | GmCmUmUmCmA-p-(ps)2-GalNAc4 | |
| | (SEQ ID NO: 63) | |
| | 3′- | |
| | mCpsmUpsmGmGfCmAmCfAmCmGmUmGmAfAm | |
| | GmCfGmAmApsfGpsd2vd3U-5′ | |
| | (SEQ ID NO: 64) | |
| ds-siNA-024 | 5′- | A |
| | mCpsmCpsmGmUfGmUfGfCfAmCmUmUmCmGmC | |
| | mUmUmCmA-p-(ps)2-GalNAc4 | |
| | (SEQ ID NO: 65) | |
| | 3′- | |
| | mCpsmUpsmGmGfCmAmCfAmCmGmUmGmAfAm | |
| | Gf(4nh)QfGmAmApsfGpsd2vd3U-5′ | |
| | (SEQ ID NO: 66) | |
| ds-siNA-025 | 5′- | A |
| | mCpsmCpsmGmUfGmUfGfCfAmCmUmUmCmGmC | |
| | mUmUmCmA-p-(ps)2GalNAc4 | |
| | (SEQ ID NO: 67) | |
| | 3′- | |
| | mCpsmUpsmGmGf(4nh)QmAmCfAmCmGmUmGm | |
| | AfAmGmCfGmAmApsfGpsd2vd3U-5′ | |
| | (SEQ ID NO: 68) | |
| ds-siNA-050* | mCpsmCpsmGmUfGmUfGfCfAmCmUmUmCmGmC | C |
| | mUmUmCmA-p-(ps)2-GalNAc4 | |
| | (SEQ ID NO: 93) | |
| | coc- | |
| | 4hUpsfGpsmAmAfGmCmGfAmAmGmUmGmCfAm | |
| | CmAfCmGmGpsm UpsmC | |
| | (SEQ ID NO: 114) | |

For EC50,
A = EC50 ≤ 0.1 nM;
B = 0.1 nM < EC50 ≤ 1 nM;
C = 1 nM < EC50 < 10;
D = EC50 ≥ 10 nM.
*ds-siNA-050 was also assessed for CC50 and the CC50 for ds-siNA-050 is >1 nm.
mX = 2′-O-methyl nucleotide;
fX = 2′-fluoro nucleotide;
5dcd3X = nucleotide of Formula 17;
5dfX = nucleotide of Formula 16;
vX = 5′ vinyl phosphonate nucleotide;
d2vX = deuterated 5’ vinyl phosphonate nucleotide;
vmX = 5′ vinyl phosphonate,
2′-O-methyl nucleotide;
omeco-d3U = 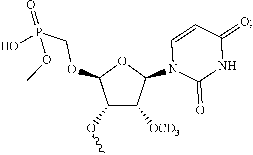
4hU = 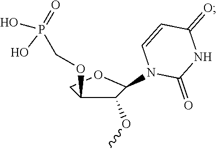
d2vd3U = 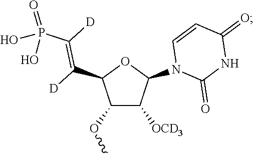
v-munU = 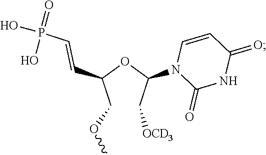
c2o-4hU = 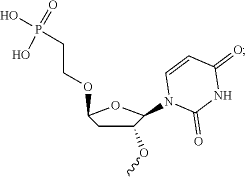
coc-4hU = 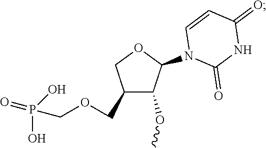
omeco-munU = 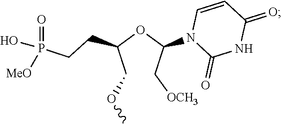
ps = phosphorothioate linkage; X is a nucleobase (e.g. A, G, C, U or T)

TABLE 8
siNA Comprising Modified Unlocked Nucleotides

| | | Position | |
|---|---|---|---|
| Name | SS/AS (5′ to 3′) | of mod. | EC₅₀ |
| CON- | mCpsmCpsmGmUfGmUfGfCfAmCmUmUmCmGmCmUmUmCmA | 6 | A |
| TROL 3 | (SEQ ID NO: 45) | | |
| | mUpsfGpsmAmAfGunCmGfAmAmGmUmGmCfAmCmAfCmGmGpsmUpsmC | | |
| | (SEQ ID NO: 46) | | |
| CON- | mCpsmCpsmGmUfGmUfGfCfAmCmUmUmCmGmCmUmUmCmA | 7 | A |
| TROL 4 | (SEQ ID NO: 47) | | |
| | mUpsfGpsmAmAfGmCunGfAmAmGmUmGmCfAmCmAfCmGmGpsmUpsmC | | |
| | (SEQ ID NO: 48) | | |
| CON- | mCpsmCpsmGmUfGmUfGfCfAmCmUmUmCmGmCmUmUmCmA | — | A |
| TROL 5 | (SEQ ID NO: 49) | | |
| | mUpsfGpsmAmAfGmCmGfAmAmGmUmGmCfAmCmAfCmGmGpsmUpsmC | | |
| | (SEQ ID NO: 50) | | |
| ds-siNA- | mCpsmCpsmGmUfGmUfGfCfAmCmUmUmCmGmCmUmUmCmA | 6 | B |
| 017 | (SEQ ID NO: 35) | | |
| | mUpsfGpsmAmAfGmun34CmGfAmAmGmUmGmCfAmCmAfCmGmGpsmUpsmC | | |
| | (SEQ ID NO: 36) | | |
| ds-siNA- | mCpsmCpsmGmUfGmUfGfCfAmCmUmUmCmGmCmUmUmCmA | 7 | A |
| 018 | (SEQ ID NO: 37) | | |
| | mUpsfGpsmAmAfGmCmun34GfAmAmGmUmGmCfAmCmAfCmGmGpsmUpsmC | | |
| | (SEQ ID NO: 38) | | |
| ds-siNA- | mCpsmCpsmGmUfGmUfGfCfAmCmUmUmCmGmCmUmUmCmA-p-ps2-GalNAc4 | 7 | A |
| 019 | (SEQ ID NO: 39) | | |
| | d2vd3UpsfGpsmAmAfGmCmun34GfAmAmGmUmGmCfAmCmAfCmGmGpsmUpsmC | | |
| | (SEQ ID NO: 40) | | |
| ds-siNA- | mCpsmCpsmun34GmUfGmUfGfCfAmCmUmUmCmGmCmUmUmCmA | 3 | A |
| 034 | (SEQ ID NO: 93) | | |
| | psmUpsfGmAmAfGmCmGfAmAmGmUmGmCfAmCmAfCmGpsmGpsmUmC | | |
| | (SEQ ID NO: 94) | | |
| ds-siNA- | mCpsmCpsmGmun34UfGmUfGfCfAmCmUmUmCmGmCmUmUmCmA | 4 | A |
| 035 | (SEQ ID NO: 95) | | |
| | psmUpsfGmAmAfGmCmGfAmAmGmUmGmCfAmCmAfCmGpsmGpsmUmC | | |
| | (SEQ ID NO: 94) | | |
| ds-siNA- | mCpsmCpsmGmUfGmun34UfGfCfAmCmUmUmCmGmCmUmUmCmA | 6 | A |
| 036 | (SEQ ID NO: 96) | | |
| | psmUpsfGmAmAfGmCmGfAmAmGmUmGmCfAmCmAfCmGpsmGpsmUmC | | |
| | (SEQ ID NO: 94) | | |
| ds-siNA- | mCpsmCpsmGmUfGmUfGfCfAmun34CmUmUmCmGmCmUmUmCmA | 10 | B |
| 037 | (SEQ ID NO: 97) | | |
| | psmUpsfGmAmAfGmCmGfAmAmGmUmGmCfAmCmAfCmGpsmGpsmUmC | | |
| | (SEQ ID NO: 94) | | |
| ds-siNA- | mCpsmCpsmGmUfGmUfGfCfAmCmun34UmUmCmGmCmUmUmCmA | 11 | A |
| 038 | (SEQ ID NO: 98) | | |
| | psmUpsfGmAmAfGmCmGfAmAmGmUmGmCfAmCmAfCmGpsmGpsmUmC | | |
| | (SEQ ID NO: 94) | | |
| ds-siNA- | mCpsmCpsmGmUfGmUfGfCfAmCmUmun34UmCmGmCmUmUmCmA | 12 | A |
| 039 | (SEQ ID NO: 99) | | |
| | psmUpsfGmAmAfGmCmGfAmAmGmUmGmCfAmCmAfCmGpsmGpsmUmC | | |
| | (SEQ ID NO: 94) | | |
| ds-siNA- | mCpsmCpsmGmUfGmUfGfCfAmCmUmUmun34CmGmCmUmUmCmA | 13 | A |
| 040 | (SEQ ID NO: 100) | | |
| | psmUpsfGmAmAfGmCmGfAmAmGmUmGmCfAmCmAfCmGpsmGpsmUmC | | |
| | (SEQ ID NO: 94) | | |
| ds-siNA- | mCpsmCpsmGmUfGmUfGfCfAmCmUmUmCmun34GmCmUmUmCmA | 14 | A |
| 041 | (SEQ ID NO: 101) | | |
| | psmUpsfGmAmAfGmCmGfAmAmGmUmGmCfAmCmAfCmGpsmGpsmUmC | | |
| | (SEQ ID NO: 94) | | |
| ds-siNA- | mCpsmCpsmGmUfGmUfGfCfAmCmUmUmCmGmun34CmUmUmCmA | 15 | A |
| 042 | (SEQ ID NO: 102) | | |
| | psmUpsfGmAmAfGmCmGfAmAmGmUmGmCfAmCmAfCmGpsmGpsmUmC | | |
| | (SEQ ID NO: 94) | | |
| ds-siNA- | mCpsmCpsmGmUfGmUfGfCfAmCmUmUmCmGmCmun34UmUmCmA | 16 | A |
| 043 | (SEQ ID NO: 103) | | |
| | psmUpsfGmAmAfGmCmGfAmAmGmUmGmCfAmCmAfCmGpsmGpsmUmC | | |
| | (SEQ ID NO: 94) | | |
| ds-siNA- | mCpsmCpsmGmUfGmUfGfCfAmCmUmUmCmGmCmUmun34UmCmA | 17 | A |
| 044 | (SEQ ID NO: 104) | | |
| | psmUpsfGmAmAfGmCmGfAmAmGmUmGmCfAmCmAfCmGpsmGpsmUmC | | |
| | (SEQ ID NO: 94) | | |
| ds-siNA- | mCpsmCpsmGmUfGmUfGfCfAmCmUmUmCmGmCmUmUmun34CmA | 18 | A |
| 045 | (SEQ ID NO: 105) | | |
| | psmUpsfGmAmAfGmCmGfAmAmGmUmGmCfAmCmAfCmGpsmGpsmUmC | | |
| | (SEQ ID NO: 94) | | |

For EC50,
A = EC50 ≤ 0.1 nM;
B = 0.1 nM < EC50 ≤ 1 nM;
C = l nM < ECso < 10;
D = EC₅₀ ≥ 10 nM.
mX = 2′--methyl nucleotide;
fX = 2′-fluoro nucleotide;
5dcd3X = nucleotide of Formula 17;
5dfX = nucleotide of Formula 16;
vX= 5′ vinyl phosphonate nucleotide;
d2vX = deuterated 5′ vinyl phosphonate nucleotide;
vmX = 5′ vinyl phosphonate, 2′-O-methyl nucleotide;
d2vd3U = 6; 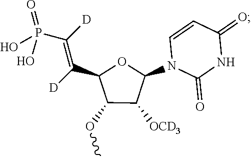
mun34C = 3; 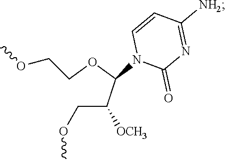
mun34G = 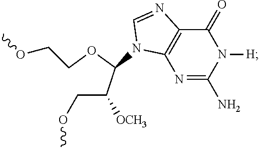
unC = 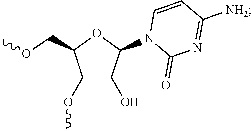
unG = 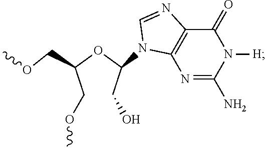
ps = phosphorothioate linkage;
X is a nucleobase (e.g. A, G, C, U or T)

TABLE 9
siNA Comprising Mesyl Phosphoroamidate Internucleoside Linkages

| Name | SS/AS (5′ to 3′) | EC₅₀ |
|---|---|---|
| ds-siNA-020 | 5′-mGpsmUpsmGmGfUmGfGfAfCmUmUmCmUmCmUmCmAmAmU-p-(ps)2-GalNAc4 | A |
| | (SEQ ID NO: 69) | |
| | 3′-mApsmGpsmCmAmCfCmAfCmCmUmGmAmAmGmAfGmAmGmUypfUypm A-5′ | |
| | (SEQ ID NO: 70) | |
| ds-siNA-021 | 5′-mGpsmUpsmGmGfUmGfGfAfCmUmUmCmUmCmUmCmAmAmU-p-(ps)2-GalNAc4 | C |
| | (SEQ ID NO: 71) | |
| | 3′-mAypmGypmCmAmCfCmAfCmCmUmGmAmAmGmAfGmAmGmUpsfUpsm A-5′ | |
| | (SEQ ID NO: 72) | |
| ds-siNA-022 | 5′-mGpsmUpsmGmGfUmGfGfAfCmUmUmCmUmCmUmCmAmAmU-p-(ps)2-GalNAc4 | C |
| | (SEQ ID NO: 73) | |
| | 3′-mAypmGypmCmAmCfCmAfCmCmUmGmAmAmGmAfGmAmGmUypfUypmA-5′ | |
| | (SEQ ID NO: 74) | |
| CONTROL 8 | 5′-mGpsmUpsmGmGfUmGfGfAfCmUmUmCmUmCmUmCmAmAmU-p-(ps)2-GalNAc4 | A |
| | (SEQ ID NO: 75) | |
| | 3′-mApsmGpsmCmAmCfCmAfCmCmUmGmAmAmGmAfGmAmGmUpsfUpsm A-5′ | |
| | (SEQ ID NO: 76) | |

For EC₅₀,
A = EC50 ≤ 0.1 nM;
B = 0.1 nM < EC₅₀ ≤ 1 nM;
C = 1 nM < EC₅₀ < 10;
D = EC₅₀ ≥ 10 nM.
mX = 2′-O-methyl nucleotide;
fX = 2′-fluoro nucleotide;
ps = phosphorothioate linkage;
X is a nucleobase (e.g. A, G, C, U or T);
yp =

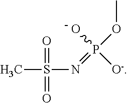

TABLE 10
siNA Comprising Modified apU Nucleotides

| Name | SS/AS (5′ to 3′) | EC₅₀ | CC₅₀ |
|---|---|---|---|
| ds-siNA-026 | mCpsmCpsmGaUfGmUfGfCfAmCmUmUmCmGmCmUmUmCmA-p-(ps)2-GalNAc4 | A | >1 |
| | (SEQ ID NO: 77) | | |
| | d2vd3UpsfGpsmAmAfGmCmGfAmAmGmUmGmCfAmCmAfCmGmGpsmUpsmC | | |
| | (SEQ ID NO: 78) | | |
| ds-siNA-027 | mCpsmCpsmGmUfGaUfGfCfAmCmUmUmCmGmCmUmUmCmA-p-(ps)2-GalNAc4 | A | >1 |
| | (SEQ ID NO: 79) | | |
| | d2vd3UpsfGpsmAmAfGmCmGfAmAmGmUmGmCfAmCmAfCmGmGpsmUpsmC | | |
| | (SEQ ID NO: 80) | | |
| ds-siNA-028 | mCpsmCpsmGmUfGmUfGfCfAmCmUaUmCmGmCmUmUmCmA-p-(ps)2-GalNAc4 | A | >1 |
| | (SEQ ID NO: 81) | | |
| | d2vd3UpsfGpsmAmAfGmCmGfAmAmGmUmGmCfAmCmAfCmGmGpsmUpsmC | | |
| | (SEQ ID NO: 82) | | |
| ds-siNA-029 | mCpsmCpsmGmUfGmUfGfCfAmCmUmUmCmGmCaUmUmCmA-p-(ps)2-GalNAc4 | A | >1 |
| | (SEQ ID NO: 83) | | |
| | d2vd3UpsfGpsmAmAfGmCmGfAmAmGmUmGmCfAmCmAfCmGmGpsmUpsmC | | |
| | (SEQ ID NO: 84) | | |
| ds-siNA-030 | mCpsmCpsmGmUfGmUfGfCfAmCmUmUmCmGmCmUaUmCmA-p-(ps)2-GalNAc4 | A | >1 |
| | (SEQ ID NO: 85) | | |
| | d2vd3UpsfGpsmAmAfGmCmGfAmAmGmUmGmCfAmCmAfCmGmGpsmUpsmC | | |
| | (SEQ ID NO: 86) | | |
| ds-siNA-031 | mCpsmCpsmGmUfGmUfGfCfAmCmUmUmCmGmCmUmUmCmA-p-(ps)2-GalNAc4 | A | >1 |
| | (SEQ ID NO: 87) | | |
| | d2vd3UpsfGpsmAmAfGmCmGfAmAmGaUmGmCfAmCmAfCmGmGpsmUpsmC | | |
| | (SEQ ID NO: 88) | | |
| ds-siNA-032 | mCpsmCpsmGmUfGmUfGfCfAmCmUmUmCmGmCmUmUmCmA-p-(ps)2-GalNAc4 | A | >1 |
| | (SEQ ID NO: 89) | | |
| | d2vd3UpsfGpsmAmAfGmCmGfAmAmGmUmGmCfAmCmAfCmGmGpsaUpsmC | | |
| | (SEQ ID NO: 90) | | |
| ds-siNA-033 | mCpsmCpsmGmUfGmUfGfCfAmCmUmUmCmGmCmUmUmCmA-p-(ps)2-GalNAc4 | A | >1 |
| | (SEQ ID NO: 91) | | |
| | aUpsfGpsmAmAfGmCmGfAmAmGmUmGmCfAmCmAfCmGmGpsmUpsmC | | |
| | (SEQ ID NO: 92) | | |

For EC₅₀,
A = EC₅₀ ≤ 0.1 nM;
B = 0.1 nM < EC₅₀ ≤ 1 nM;
C = 1 nM < EC₅₀ < 10;
D = EC₅₀ ≥ 10 nM.
mX = 2′-O-methyl nucleotide;
fX = 2′-fluoro nucleotide;
5dcd3X = nucleotide of Formula 17;
5dfX = nucleotide of Formula 16;
vX = 5′ vinyl phosphonate nucleotide;
d2vX = deuterated 5′
vinyl phosphonate nucleotide;
vmX = 5′ vinyl phosphonate, 2′-O-methyl nucleotide;
d2vd3U = 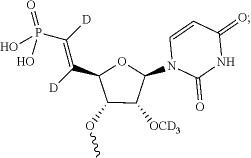
aU = 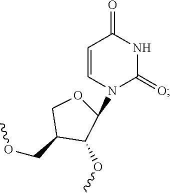
ps = phosphorothioate linkage;
X is a nucleobase (e.g. A, G, C, U or T)

### In Vivo Assay:

AAV/HBV is a recombinant AAV carrying replicable HBV genome. Taking advantage of the highly hepatotropic feature of genotype 8 AAV, the HBV genome can be efficiently delivered to the mouse liver cells. Infection of immune competent mouse with AAV/HBV can result in long term HBV viremia, which mimics chronic HBV infection in patients. The AAV/HBV model can be used to evaluate the in vivo activity of various types of anti-HBV agents. Mice were infected with AAV-HBV on day −28 of the study. The test articles or negative control (PBS) were dosed subcutaneously (unless specified otherwise) as single dose on days 0 at 5 mg/kg. Serial blood collections were usually taken every 5 days on day 0, 5, 10 and 15 etc. until the termination of studies. Serum HBV S antigen (HBsAg) was assayed through ELISA.

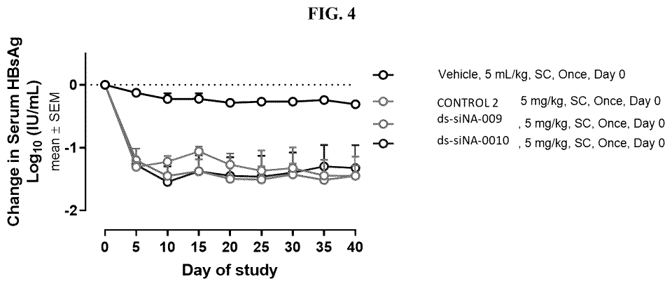

Table 11 shows the siNA that were assessed to determine the impact of some of the exemplary nucleotide phosphate mimics. The results of this assessment are shown in FIG. 4, which provides a graph of the change in serum HBsAg from AAV-HBV mice treated with vehicle (G01), CONTROL 2, ds-siNA-009, or ds-siNA-010.

TABLE 11

| Name | SS/AS 5′ to 3′ |
|---|---|
| CONTROL 2 | mCpsmCpsmGmUfGmUfGfCfAmCmUmUmCmGm |
| | CmUmUmCmAp-ps2-GalNAc4 |
| | (SEQ ID NO: 43) |
| | mUpsfGpsmAmAfGmCmGfAmAmGmUmGmCfAm |
| | CmAfCmGmGpsmUpsmC |
| | (SEQ ID NO: 44) |
| ds-siNA-009 | mCpsmCpsmGmUfGmUfGfCfAmCmUmUmCmGm |
| | CmUmUmCmAp-ps2-GalNAc4 |
| | (SEQ ID NO: 17) |
| | omeco-d3UpsfGpsmAmAfGmCmGfAmAmGmUm |
| | GmCfAmCmAfCmGmGpsmUpsmC |
| | (SEQ ID NO: 18) |
| ds-siNA-010 | mCpsmCpsmGmUfGmUfGfCfAmCmUmUmCmGm |
| | CmUmUmCmAp-ps2-GalNAc4 |
| | (SEQ ID NO: 19) |
| | 4hUpsfGpsmAmAfGmCmGfAmAmGmUmGmCfAm |
| | CmAfCmGmGpsmUpsmC |
| | (SEQ ID NO: 20) |

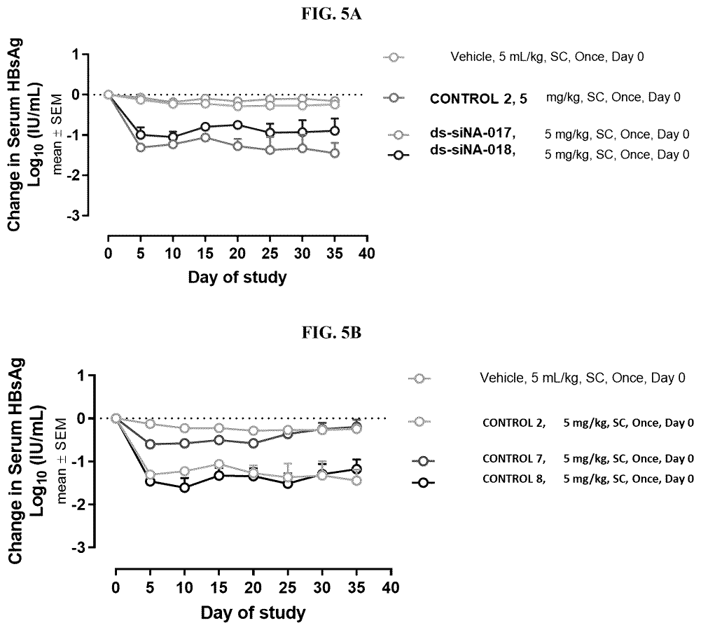

Table 12 shows the siNA that were assessed to determine the impact of some of the exemplary nucleotide phosphate mimics. The results of this assessment are shown in FIG. 5A, which provides a graph of the change in serum HIBsAg from AAV-HIBV mice treated with vehicle (GO 1), CONTROL 2, ds-siNA-017 (with the addition of a GalNAc), or ds-siNA-018 (with the addition of a GalNAc).

TABLE 12

| Name | SS/AS 5′ to 3′ |
|---|---|
| ds-siNA-0017 | mCpsmCpsmGmUfGmUfGfCfAmCmUmUmCmGm |
| | CmUmUmCmA-p-ps2-GalNac4 |
| | (SEQ ID NO: 35) |
| | mUpsfGpsmAmAfGmun34CmGfAmAmGmUmGm |
| | CfAmCmAfCmGmGpsmUpsmC |
| | (SEQ ID NO: 36) |
| ds-siNA-0018 | mCpsmCpsmGmUfGmUfGfCfAmCmUmUmCmGm |
| | CmUmUmCmA-p-ps2-GalNac4 |
| | (SEQ ID NO: 37) |
| | mUpsfGpsmAmAfGmCmun34GfAmAmGmUmGm |
| | CfAmCmAfCmGmGpsmUpsmC |
| | (SEQ ID NO: 38) |
| CONTROL 2 | mCpsmCpsmGmUfGmUfGfCfAmCmUmUmCmGm |
| | CmUmUmCmAp-ps2-GalNAc4 |
| | (SEQ ID NO: 43) |
| | mUpsfGpsmAmAfGmCmGfAmAmGmUmGmCfAm |
| | CmAfCmGmGpsmUpsmC |
| | (SEQ ID NO: 44) |

Table 13 shows siAN comprising traditional UNA that were also assessed. These siNA can be considered controls of the novel 3′,4′ seco modified nucleotides disclosed herein. FIG. 5B provides a graph of the change in serum HIBsAg from AAV-HIBV mice treated with vehicle (G01), CONTROL 2, CONTROL 7, or CONTROL 8.

TABLE 13

| Name | SS/AS |
|---|---|
| CONTROL 7 | mCpsmCpsmGmUfGmUfGfCfAmCmUmUmCmGmCm |
| | UmUmCmA-p-ps2-GalNAc4 |
| | (SEQ ID NO: 51) |
| | mUpsfGpsmAmAfGunCmGfAmAmGmUmGmCfAm |
| | CmAfCmGmGpsmUpsmC |
| | (SEQ ID NO: 52) |
| CONTROL 8 | mCpsmCpsmGmUfGmUfGfCfAmCmUmUmCmGmCm |
| | UmUmCmA-p-ps2-GalNac4 |
| | (SEQ ID NO: 53) |
| | mUpsfGpsmAmAfGmCunGfAmAmGmUmGmCfAm |
| | CmAfCmGmGpsmUpsmC |
| | (SEQ ID NO: 54) |

Table 14 shows the siNA that were assessed to determine the impact of some of the exemplary nucleotide phosphate mimics. The results of this assessment are shown in FIG. 6, which provides a graph of the change in serum HIBsAg from AAV-HIBV mice treated with vehicle (G01), CONTROL 2, ds-siNA-01 1, ds-siNA-012, or ds-siNA-013.

TABLE 14

| Name | SS/AS 5′ to 3′ |
|---|---|
| CONTROL 2 | mCpsmCpsmGmUfGmUfGfCfAmCmUmUmCmGm |
| | CmUmUmCmAp-ps2-GalNAc4 |
| | (SEQ ID NO: 43) |
| | mUpsfGpsmAmAfGmCmGfAmAmGmUmGmCfAm |
| | CmAfCmGmGpsmUpsmC |
| | (SEQ ID NO: 44) |
| ds-siNA-011 | mCpsmCpsmGmUfGmUfGfCfAmCmUmUmCmGm |
| | CmUmUmCmAp-ps2-GalNAc4 |
| | (SEQ ID NO: 21) |
| | d2vd3UpsfGpsmAmAfGmCmGfAmAmGmUmGm |
| | CfAmCmAfCmGmGpsmUpsmC |
| | (SEQ ID NO: 22) |
| ds-siNA-012 | mCpsmCpsmGmUfGmUfGfCfAmCmUmUmCmGm |
| | CmUmUmCmA-p-ps2-GalNAc4 |
| | (SEQ ID NO: 23) |
| | v-munUpsfGpsmAmAfGmCmGfAmAmGmUmGm |
| | CfAmCmAfCmGmGpsmUpsmC |
| | (SEQ ID NO: 24) |
| ds-siNA-013 | mCpsmCpsmGmUfGmUfGfCfAmCmUmUmCmGm |
| | CmUmUmCmA-p-ps2-GalNAc4 |
| | (SEQ ID NO: 25) |
| | c2o-4hUpsfGpsmAmAfGmCmGfAmAmGmUm |
| | GmCfAmCmAfCmGmGpsmUpsmC |
| | (SEQ ID NO: 26) |

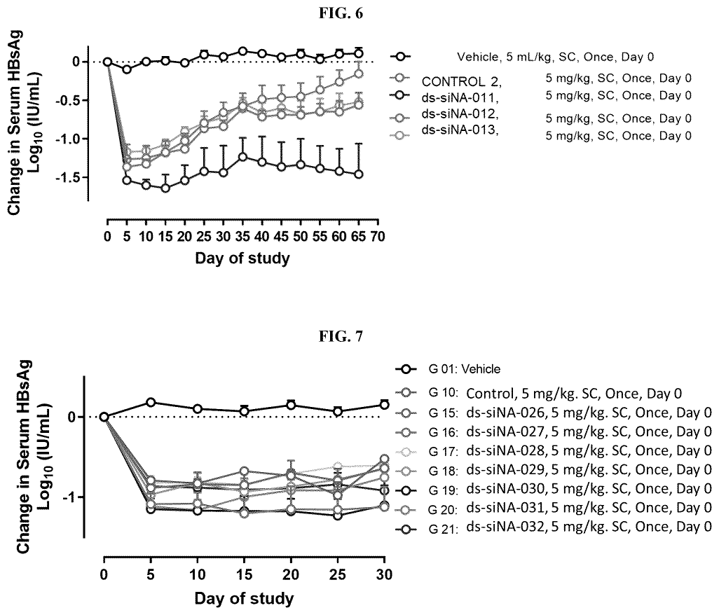

Table 15 shows the siNA that were assessed to determine the impact of incorporation of an apU nucleotide. The results of this assessment are shown in FIG. 7, which provides a graph of the change in serum HIBsAg from AAV-HIBV mice treated with vehicle (G01), CONTROL 2, ds-siNA-026, ds-siNA-027, ds-siNA-028, ds-siNA-029, ds-siNA-030, ds-siNA-031, or ds-siNA-032.

TABLE 15

| Name | SS/AS 5′ to 3′ |
|---|---|
| CONTROL 2 | mCpsmCpsmGmUfGmUfGfCfAmCmUmUmCmGm |
| | CmUmUmCmAp-ps2-GalNAc4 |
| | (SEQ ID NO: 43) |
| | mUpsfGpsmAmAfGmCmGfAmAmGmUmGmCfAm |
| | CmAfCmGmGpsmUpsmC |
| | (SEQ ID NO: 44) |
| ds-siNA-026 | mCpsmCpsmGaUfGmUfGfCfAmCmUmUmCmGm |
| | CmUmUmCmA-p-(ps)2-GalNAc4 |
| | (SEQ ID NO: 77) |
| | d2vd3UpsfGpsmAmAfGmCmGfAmAmGmUmGm |
| | CfAmCmAfCmGmGpsmUpsmC |
| | (SEQ ID NO: 78) |
| ds-siNA-027 | mCpsmCpsmGmUfGaUfGfCfAmCmUmUmCmGm |
| | CmUmUmCmA-p-(ps)2-GalNAc4 |
| | (SEQ ID NO: 79) |
| | d2vd3UpsfGpsmAmAfGmCmGfAmAmGmUmGm |
| | CfAmCmAfCmGmGpsmUpsmC |
| | (SEQ ID NO: 80) |
| ds-siNA-028 | mCpsmCpsmGmUfGmUfGfCfAmCmUaUmCmGm |
| | CmUmUmCmA-p-(ps)2-GalNAc4 |
| | (SEQ ID NO: 81) |
| | d2vd3UpsfGpsmAmAfGmCmGfAmAmGmUmGm |
| | CfAmCmAfCmGmGpsmUpsmC |
| | (SEQ ID NO: 82) |
| ds-siNA-029 | mCpsmCpsmGmUfGmUfGfCfAmCmUmUmCmGm |
| | CaUmUmCmA-p-(ps)2-GalNAc4 |
| | (SEQ ID NO: 83) |
| | d2vd3UpsfGpsmAmAfGmCmGfAmAmGmUmGm |
| | CfAmCmAfCmGmGpsmUpsmC |
| | (SEQ ID NO: 84) |
| ds-siNA-030 | mCpsmCpsmGmUfGmUfGfCfAmCmUmUmCmGm |
| | CmUaUmCmA-p-(ps)2-GalNAc4 |
| | (SEQ ID NO: 85) |
| | d2vd3UpsfGpsmAmAfGmCmGfAmAmGmUmGm |
| | CfAmCmAfCmGmGpsmUpsmC |
| | (SEQ ID NO: 86) |
| ds-siNA-031 | mCpsmCpsmGmUfGmUfGfCfAmCmUmUmCmGm |
| | CmUmUmCmA-p-(ps)2-GalNAc4 |
| | (SEQ ID NO: 87) |
| | d2vd3UpsfGpsmAmAfGmCmGfAmAmGaUmGm |
| | CfAmCmAfCmGmGpsmUpsmC |
| | (SEQ ID NO: 88) |
| ds-siNA-032 | mCpsmCpsmGmUfGmUfGfCfAmCmUmUmCmGm |
| | CmUmUmCmA-p-(ps)2-GalNAc4 |
| | (SEQ ID NO: 89) |
| | d2vd3UpsfGpsmAmAfGmCmGfAmAmGmUmGm |
| | CfAmCmAfCmGmGpsaUpsmC |
| | (SEQ ID NO: 90) |

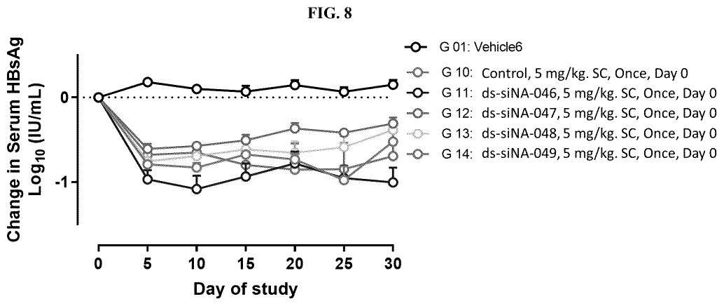

Additionally, the most active compounds from the in vitro screening of ds-siNA-034 to ds-siNA-045 were further modified to attach a GalNAc to the 3′ end of the sense strand and incorporated a deuterated vinyl phosphonate into the antisense strand. The most active compounds among ds-siNA-034 to ds-siNA-045 were ds-siNA-034 (mun34 at position 3 of sense strand), ds-siNA-043 (mun34 at position 16 of sense strand), ds-siNA-044 (mun34 at position 17 of sense strand), and ds-siNA-045 (mun34 at position 18 of sense strand). The GalNAc-conjugated/deuterated versions of these compounds were assigned ds-siNA-046 to ds-siNA-049 (shown in Table 16) and FIG. 8 provides a graph of the change in serum HBsAg from AAV-HBV mice treated with vehicle (G01), CONTROL 2, ds-siNA-046, ds-siNA-047, ds-siNA-048, or ds-siNA-049.

TABLE 16

| Name | SS/AS 5′ to 3′ |
|---|---|
| CONTROL 2 | mCpsmCpsmGmUfGmUfGfCfAmCmUmUmCmGm |
| | CmUmUmCmAp-ps2-GalNAc4 |
| | (SEQ ID NO: 43) |
| | mUpsfGpsmAmAfGmCmGfAmAmGmUmGmCfAm |
| | CmAfCmGmGpsmUpsmC |
| | (SEQ ID NO: 44) |
| ds-si-NA-046 | mCpsmCpsmun34GmUfGmUfGfCfAmCmUmUm |
| | CmGmCmUmUmCmA-p-(ps)2-GalNAc |
| | (SEQ ID NO: 106) |
| | psd2vd3UpsfGmAmAfGmCmGfAmAmGmUmGm |
| | CfAmCmAfCmGpsmGpsmUmC |
| | (SEQ ID NO: 107) |
| ds-si-NA-047 | mCpsmCpsmGmUfGmUfGfCfAmCmUmUmCmGm |
| | Cmun34UmUmCmA-p-(ps)2-GalNAc |
| | (SEQ ID NO: 108) |
| | psd2vd3UpsfGmAmAfGmCmGfAmAmGmUmGm |
| | CfAmCmAfCmGpsmGpsmUmC |
| | (SEQ ID NO: 107) |
| ds-si-NA-048 | mCpsmCpsmGmUfGmUfGfCfAmCmUmUmCmGm |
| | CmUmun34UmCmA-p-(ps)2-GalNAc |
| | (SEQ ID NO: 109) |
| | psd2vd3UpsfGmAmAfGmCmGfAmAmGmUmGm |
| | CfAmCmAfCmGpsmGpsmUmC |
| | (SEQ ID NO: 107) |
| ds-si-NA-049 | mCpsmCpsmGmUfGmUfGfCfAmCmUmUmCmGm |
| | CmUmUmun34CmA-p-(ps)2-GalNAc |
| | (SEQ ID NO: 110) |
| | psd2vd3UpsfGmAmAfGmCmGfAmAmGmUmGm |
| | CfAmCmAfCmGpsmGpsmUmC |
| | (SEQ ID NO: 107) |

### Example 22: Preparation of Compound 40-9 (GalNAc4 Amidite)

Compound 40-9 can be conjugated to any siNA disclosed herein as a targeting moiety. This compound, pictured below, can be prepared according to the following brief description.

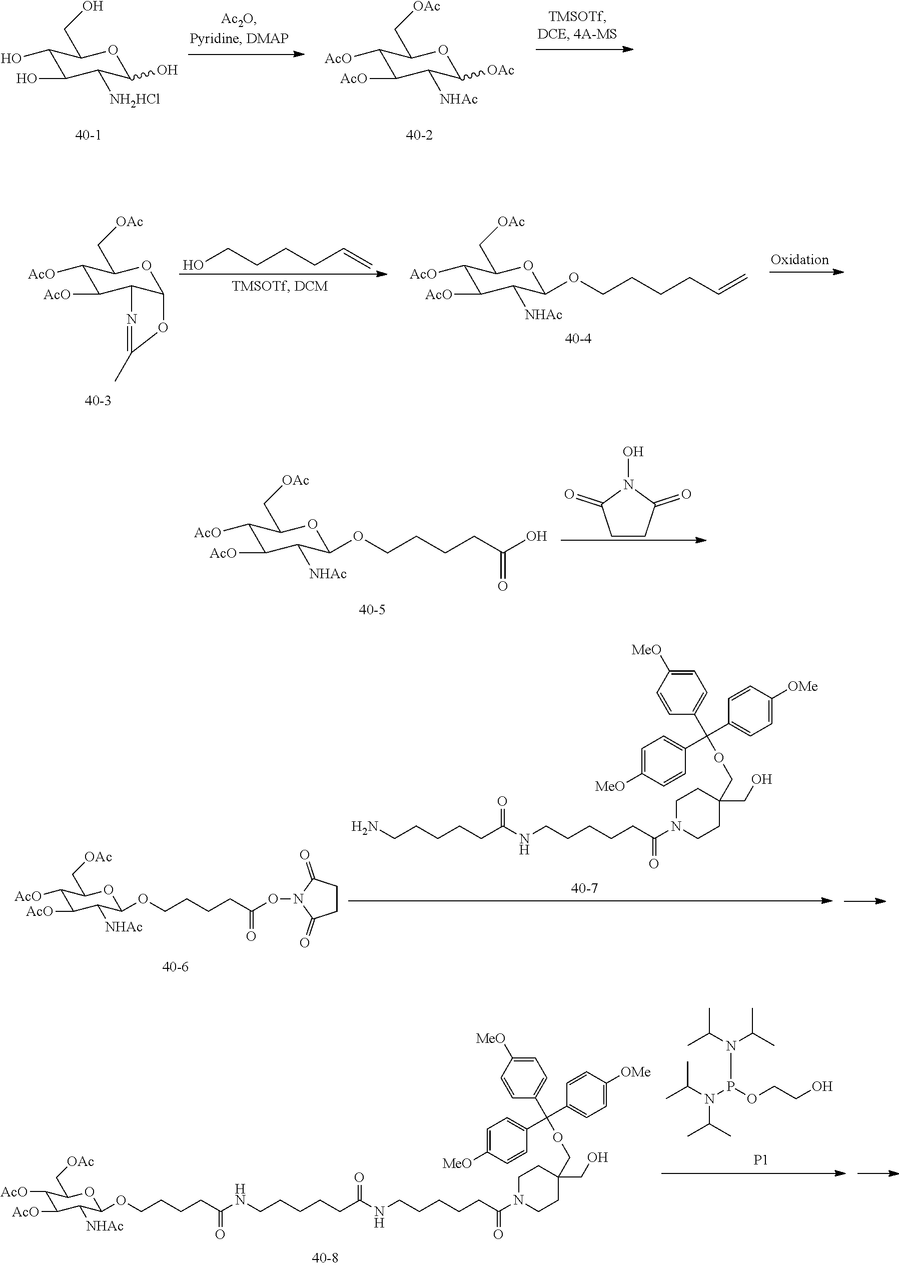

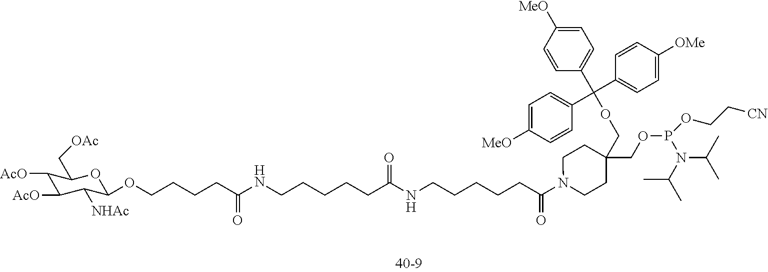 [SMILES: COc1ccc(C(OCC2(COP(OCCC#N)N(C(C)C)C(C)C)CCN(C(=O)CCCCCNC(=O)CCCCCNC(=O)CCCCOC3OC(COC(C)=O)C(OC(C)=O)C(OC(C)=O)C3NC(C)=O)CC2)(c2ccc(OC)cc2)c2ccc(OC)cc2)cc1]

The building block compound 40-9 is useful for making embodiments of modified phosphorothioated oligonucleotides. The compound 40-9 was prepared as follows:

Preparation of compound 40-2: To a solution of commercially available glucosamine hydrochloride 40-1 (60 g, 278.25 mmol, 1 eq) in DCM (300 mL) at 0° C. was added Ac₂₀ (323.83 g, 3.17 mol, 297.09 mL, 11.4 eq) dropwise, followed by pyridine (300 mL) and DMAP (3.40 g, 27.83 mmol, 0.1 eq). The mixture was allowed to gradually warm to 20° C. and stirred at 20° C. for 24 hours. Upon completion as monitored by LCMS, the mixture was concentrated under reduced pressure, diluted with DCM (900 mL), and extracted with NaHCO₃ (sat., aqueous 300 mL*3). The combined organic layers were washed with brine (300 mL), dried over Na₂SO₄, filtered and concentrated under reduced pressure to give compound 40-2 (89.5 g, crude) as a yellow solid. ¹H NMR (400 MHz, CDCl₃) δ=6.16 (d, J=3.8 Hz, 1H), 5.62 (d, J=9.0 Hz, 1H), 5.27-5.16 (m, 2H), 4.54-4.43 (m, 1H), 4.24 (dd, J=4.0, 12.5 Hz, 1H), 4.10-3.94 (m, 2H), 2.18 (s, 3H), 2.08 (s, 3H), 2.04 (d, J=4.0 Hz, 6H), 1.93 (s, 3H; LCMS (ESI): m/z calcd. for C₁₆H₂₃NaNO₁₀412.34 [M+Na]⁺, found 412.0).

Preparation of compound 40-3: To a solution of compound 40-2 (40 g, 102.73 mmol, 1 eq) in DCE (320 mL) at 25° C. was added dropwise TMSOTf (23.98 g, 107.87 mmol, 19.49 mL, 1.05 eq), and the mixture was stirred at 60° C. for 4 hours. Upon completion as monitored by LCMS, the mixture was quenched by addition of TEA (60 mL) at 20° C., stirred for 15 min, diluted with DCM (500 mL), and washed with NaHCO₃ (sat., aqueous 300 mL*2). The organic layer was washed with brine (300 mL), dried over Na₂SO₄, filtered, and concentrated under reduced pressure to give compound 40-3 (32.5 g, crude) as a yellow oil. ¹H NMR (400 MHz, CDCl₃) δ=5.96 (d, J=7.3 Hz, 1H), 5.25 (t, J=2.4 Hz, 1H), 4.95-4.88 (m, 1H), 4.19-4.08 (m, 3H), 3.59 (m, 1H), 2.13-2.05 (m, 12H).

Preparation of compound 40-4: To a mixture of compound 40-3 (32.5 g, 98.69 mmol, 1 eq) in DCM (250 mL) was added hex-5-en-1-ol (11.86 g, 118.43 mmol, 13.96 mL, 1.2 eq) and 4A MS (32.5 g). The mixture was stirred at 30° C. for 0.5 h, followed by dropwise addition of TMSOTf (13.16 g, 59.22 mmol, 10.70 mL, 0.6 eq). The mixture was stirred at 30° C. for 16 hours. Upon completion as monitored by LCMS, the reaction mixture was filtered, and the filtrate was diluted with DCM (300 mL) and washed with NaHCO₃ (sat., aqueous 150 mL*2). The organic layer was washed with brine (150 mL), dried over Na₂SO₄, filtered and concentrated under reduced pressure. The residue was purified by flash silica gel chromatography (ISCO®; 220 g SepaFlash® Silica Flash Column, Eluent of 0˜70% PE/EA gradient at 100 mL/min) to give compound 40-4 (12.3 g, 28.64 mmol, 29.02% yield) as a white solid. ¹H NMR (400 MHz, CDCl₃) δ=5.78 (m, 1H), 5.45 (d, J=8.8 Hz, 1H), 5.31 (dd, J=9.4, 10.7 Hz, 1H), 5.06 (t, J=9.5 Hz, 1H), 5.02-4.92 (m, 2H), 4.68 (d, J=8.3 Hz, 1H), 4.30-4.23 (m, 1H), 4.16-4.10 (m, 1H), 3.91-3.76 (m, 2H), 3.73-3.66 (m, 1H), 3.48 (td, J=6.7, 9.5 Hz, 1H), 2.09-2.01 (m, 11H), 1.94 (s, 3H), 1.60-1.36 (m, 4H); LCMS (ESI): m/z calcd. for C₂₀H₃₂NO₉, 430.47 [M+H]⁺, found 430.1.

Preparation of compound 40-5: To a solution of compound 40-4 (12.3 g, 28.64 mmol, 1 eq) in a mixed solvent of DCM (60 mL) and MeCN (60 mL) was added NaIO4 (2.5 M, 57.28 mL, 5 eq), and the mixture was stirred at 20° C. for 0.5 hours. RuCl₃ (123.00 mg, 592.97 umol, 0.02 eq) was added, and the mixture was stirred at 20° C. for 2 hours. Upon completion as monitored by LCMS, saturated aqueous NaHCO₃ was added to the mixture to adjust pH>7. The mixture was diluted with DCM (300 mL) and subjected to extraction. The aqueous layer was adjusted to pH<7 by citric acid, and the aqueous layer was extracted with DCM (300 mL*3). The combined organic layers were washed with brine (300 mL), dried over Na₂SO₄, filtered, and concentrated under reduced pressure to give compound 40-5 (8.9 g, 69.31% yield, as a brown solid. ¹H NMR (400 MHz, CDCl₃) δ=6.14 (d, J=8.8 Hz, 1H), 5.34-5.20 (m, 1H), 5.08-5.01 (m, 1H), 4.67 (d, J=8.3 Hz, 1H), 4.24 (dd, J=4.8, 12.3 Hz, 1H), 4.17-4.05 (m, 1H), 3.90-3.83 (m, 2H), 3.75-3.62 (m, 2H), 3.50 (d, J=5.9, 9.9 Hz, 1H), 2.44-2.27 (m, 2H), 2.09-1.93 (m, 12H), 1.75-1.53 (m, 4H); LCMS (ESI): m/z calcd. for C₁₉H₃₀NO₁₁, 448.44 [M+H]⁺, found 448.1.

Preparation of compound 40-6: To a solution of compound 40-5 (10 g, 22.35 mmol, 1 eq) and 1-hydroxypyrrolidine-2,5-dione (2.83 g, 24.58 mmol, 1.1 eq) in DCM (100 mL) was added EDCl HCl (5.57 g, 29.05 mmol, 1.3 eq), and the mixture was stirred at 20° C. for 2 hour. Upon completion as monitored by LCMS, the reaction mixture was diluted with DCM (200 mL) and washed with H₂O (100 mL). The organic layer was washed with NaHCO₃ (sat. aqueous) (100 mL*2) and brine (100 mL), dried over Na₂SO₄, filtered, and concentrated under reduced pressure to give compound 40-6 (10.1 g, 82.66%) as a white solid. ¹H NMR (400 MHz, CDCl₃) δ=5.85 (d, J=8.8 Hz, 1H), 5.31-5.26 (m, 1H), 5.06 (t, J=9.7 Hz, 1H), 4.69 (d, J=8.3 Hz, 1H), 4.25 (dd, J=4.7, 12.2 Hz, 1H), 4.12 (dd, J=2.3, 12.2 Hz, 1H), 3.94-3.79 (m, 2H), 3.75-3.65 (m, 1H), 3.63-3.53 (m, 1H), 2.87 (br d, J=4.3 Hz, 4H), 2.76-2.56 (m, 2H), 2.08 (s, 3H), 2.02 (d, J=1.8 Hz, 6H), 1.92 (s, 3H), 1.86-1.66 (m, 4H); LCMS (ESI): m/z calcd. for C₂₃H₃₃N₂O₁₃, 545.51 [M+H]⁺, found 545.1.

Preparation of compound 40-8: To a solution of compound 40-7 (40-7 prepared by following the general procedure described in WO 2018013999 Al) (9.8 g, 13.92 mmol, 1 eq) in DCM (100 mL) was added DIEA (3.60 g, 27.84 mmol, 4.85 mL, 2 eq), followed by addition of (2,5-dioxopyrrolidin-1-yl) 5-[3-acetamido-4,5-diacetoxy-6-(acetoxymethyl)tetrahydropyran-2-yl]oxypentanoate (compound 40-6) (9.86 g, 18.10 mmol, 1.3 eq), and the mixture was stirred at 20° C. for 2 hours. Upon completion as monitored by LCMS, the reaction mixture was diluted with water (100 mL), and then extracted with DCM (100 mL*2). The combined organic layers were washed brine (100 mL), dried over anhydrous Na₂SO₄, filtered and the filtrate was concentrated under reduced pressure to give a residue. The residue was purified by flash silica gel chromatography (ISCO®; 120 g SepaFlash® Silica Flash Column, Eluent of 0˜6% MeOH/DCM gradient at 80 mL/min) to give compound 40-8 (13.1 g, 80.95% yield,) as a white solid. ¹H NMR (400 MHz, DMSO-d6) δ=8.06 (d, J=9.3 Hz, 1H), 7.81 (q, J=5.4 Hz, 2H), 7.21 (d, J=8.8 Hz, 6H), 6.84 (d, J=9.0 Hz, 6H), 5.04 (t, J=10.0 Hz, 1H), 4.78 (t, J=9.7 Hz, 1H), 4.55 (d, J=8.5 Hz, 1H), 4.17 (dd, J=4.5, 12.3 Hz, 1H), 3.97 (d, J=10.0 Hz, 1H), 3.77 (dd, J=2.6, 9.9 Hz, 1H), 3.72-3.64 (m, 11H), 3.46-3.25 (m, 5H), 3.05-2.84 (m, 8H), 2.18 (t, J=7.2 Hz, 2H), 2.05-1.95 (m, 7H), 1.93 (s, 3H), 1.88 (s, 3H), 1.74 (s, 3H), 1.47-1.13 (m, 20H); LCMS (ESI): RT=2.017 min, m/z calcd. for C₆₀H₈₄NaN₄O₁₇, 1156.32[M+Na]₊, 1155.5.

Preparation of compound 40-9: To a mixture of compound 40-8 (5 g, 4.41 mmol, 1 eq) and 4A MS (5 g) in DCM (50 mL) was added 3-bis(diisopropylamino)phosphanyloxypropanenitrile (1.73 g, 5.74 mmol, 1.82 mL, 1.3 eq) at −10° C., followed by addition of 1H-imidazole-4,5-dicarbonitrile (573.12 mg, 4.85 mmol, 1.1 eq), and the mixture was stirred at 0° C. for 2 hours. Upon completion as monitored by LCMS, the reaction mixture was diluted with DCM (100 mL), washed with NaHCO₃ (sat., aqueous, 50 mL*2), dried over Na₂SO₄, and concentrated under reduced pressure to give a pale yellow foam. The residue was purified by flash silica gel chromatography (ISCO®; 40 g SepaFlash® Silica Flash Column, 0% to 10% i-PrOH in DCM contain 2% TEA) to give compound 40-9 (3.35 g, 56.60% yield,) as a white solid. ¹H NMR (400 MHz, CD₃CN) 6=7.35-7.25 (m, 6H), 6.88-6.82 (m, 6H), 6.79 (d, J=9.3 Hz, 1H), 6.63-6.46 (m, 2H), 5.17-5.08 (m, 1H), 4.93 (t, J=9.7 Hz, 1H), 4.59 (d, J=8.6 Hz, 1H), 4.22 (dd, J=4.9, 12.2 Hz, 1H), 4.04 (dd, J=2.4, 12.2 Hz, 1H), 3.85-3.32 (m, 22H), 3.15-3.00 (m, 8H), 2.59 (t, J=5.8 Hz, 2H), 2.23 (br t, J=6.6 Hz, 3H), 2.12-2.04 (m, 4H), 2.00 (s, 3H), 1.96 (s, 3H), 1.93 (s, 3H), 1.82 (s, 3H), 1.66-1.45 (m, 12H), 1.42-1.21 (m, 6H), 1.19-1.07 (m, 12H); LCMS (ESI) m/z calcd. for C₆₉H₁₀₁NaN₆O₁₈P 1355.68 [M+Na]+, found 1355.7; ³¹P NMR (CD₃CN) 6=147.00.

### Example 23: Preparation of GalNAc4 CPG

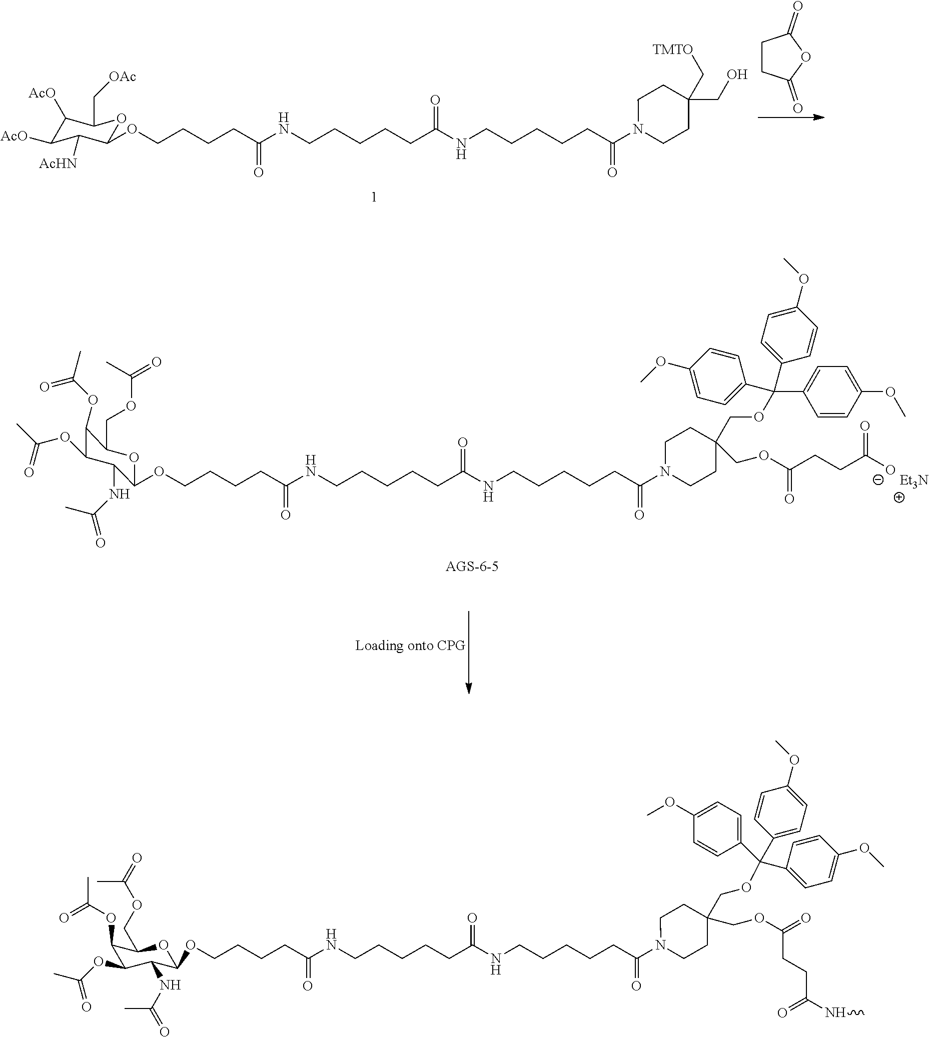

To a solution of 40-8 (21 g, 18.53 mmol, 1 eq) and succinic anhydride (9.27 g, 92.65 mmol, 5 eq) in DCM (160 mL) were added TEA (18.75 g, 185.30 mmol, 25.79 mL, 10 eq) and DMAP (2.26 g, 18.53 mmol, 1 eq) at 15° C. The mixture was stirred at 15° C. for 16 h. TLC (DCM:MeOH=10:1) showed the reaction was complete. The reaction mixture was diluted with water (200 mL), and then extracted with DCM (300 mL*2). The combined organic layers were washed with brine (300 mL*3), dried over anhydrous Na₂SO₄, concentrated under reduced pressure. The residue was purified by flash silica gel chromatography (ISCO®; 220 g SepaFlash® Silica Flash Column, Eluent of 0˜10% MeOH/DCM/TEA DCM was added 0.5 @100 mL/min) to give AGS-6-5 (12.8 g, 56% yield) LCMS: (ESI): m/z 1233.6 [M+H]⁺. Further Succinate AGS-6-5 was loaded onto LCAA (CNA) 500 A CPG by following the general procedure to give GalNAc 4 CPG.

### Example 24: Synthesis of Monomer

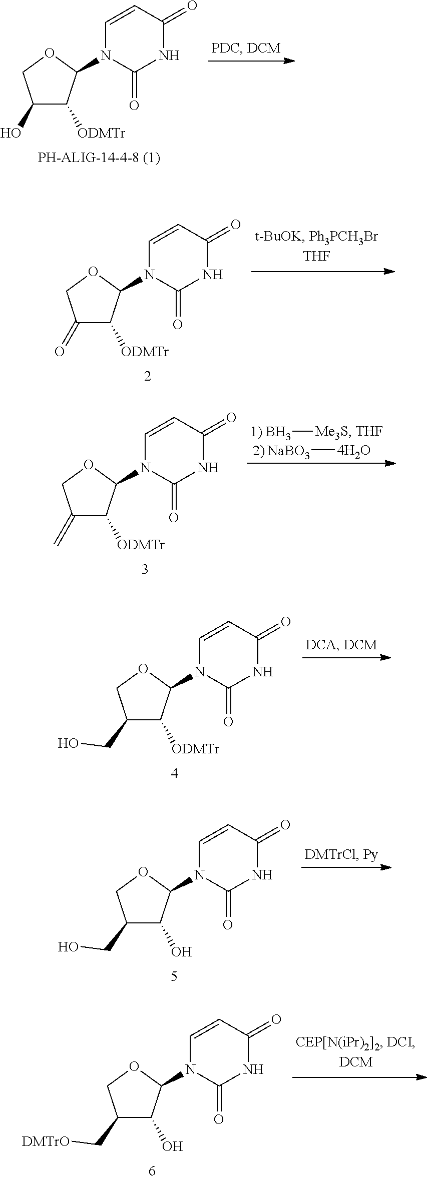

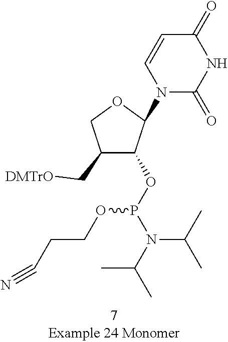

Preparation of (2): Into a 1000 mL round-bottom flask were added PDC (8.48 g, 22.53 mmol, 1.2 equiv) and 4A-MS (18 g) and DCM (300 mL) and PH-ALG-14-4-8 (from example 5) (9.7 g, 18.8 mmol) at room temperature. The resulting mixture was stirred for 5 h at room temperature under argon atmosphere. The resulting mixture was diluted with ethyl acetate (30 mL). The resulting mixture was filtered, the filter cake was washed with ethyl acetate (4×30 mL). The filtrate was concentrated under reduced pressure. This resulted 2 (9 g, crude) as a yellow solid. LC-MS: m/z 513.2 [M−H]⁻

Preparation of (3): Into a 1000 mL 3-necked round-bottom flask were added methyl triphenyl-phosphonium bromide (15.62 g, 43.73 mmol) and THE (180 mL) and t-BuOK (43.7 mL, 2M in TIF) at 0° C. The resulting mixture was stirred for 30 min at 0° C. under argon atmosphere. To a stirred mixture, 3′-ketone 2 (9 g, 17.49 mmol) in THE was added dropwise at 0° C. under argon atmosphere. The resulting mixture was stirred for 2 h at room temperature under argon atmosphere. The reaction was quenched by the addition of water (1 mL) at room temperature. The resulting mixture was filtered, the filter cake was washed with ethyl acetate (4×20 mL). The filtrate was washed with water (3×20 mL), dried over anhydrous Na₂SO₄. After filtration, the filtrate was concentrated under reduced pressure. The residue was purified by reverse flash chromatography with the following conditions: column, C18; mobile phase, ACN in water, 45% to 70% gradient in 30 min; detector, UV 254 to afford 3 (5.6 g, 56% yield in two steps) as a white solid. LC-MS: m/z 511.15 [M−H]⁻; ¹H-NMR (400 MHz, DMSO-d6) δ 11.26 (s, 1H), 7.38 (d, J=7.3 Hz, 2H), 7.33-7.18 (m, 8H), 6.90-6.78 (m, 4H), 5.71 (d, J=3.7 Hz, 1H), 5.42 (dd, J=8.1, 1.9 Hz, 1H), 4.96 (d, J=2.1 Hz, 1H), 4.88 (d, J=3.6 Hz, 1H), 4.47 (d, J=15.1 Hz, 3H), 3.72 (d, J=3.8 Hz, 6H).

Preparation of (4): Into a 500 mL round-bottom flask were added intermediate 3 (5.5 g, 10.73 mmol) and THE (140 mL) and BH₃-Me₂S (24.14 mL, 48.28 mmol) at 0° C. The resulting mixture was stirred for 5 days at 0° C. under argon atmosphere. To a stirred mixture, MeOH (56 mL) was added dropwise at 0° C. under argon atmosphere. The resulting mixture was stirred for 20 min at 0° C. under argon atmosphere. To a stirred mixture, H₂O (84 mL) were added dropwise at 0° C. under argon atmosphere. Further NaBO_{3·}4H₂O (29.7 g, 193.14 mmol) were added in several batches at 0° C. under argon atmosphere. The resulting mixture was stirred for 1 day at room temperature under argon atmosphere. The resulting mixture was diluted with ethyl acetate (200 mL). The resulting mixture was extracted with EtOAc (3×200 mL). The combined organic layers were washed with brine (2×100 mL), dried over anhydrous Na₂SO₄. After filtration, the filtrate was concentrated under reduced pressure. The residue was purified by reverse flash chromatography with the following conditions: column, C18; mobile phase, ACN in water, 35% to 70% gradient in 30 min; detector, UV 254 nm. to give compound 4 (1.4 g, 24% yield) as a white solid. LC-MS: m/z 529.15 [M−H]⁻; ¹H-NMR: (400 MHz, DMSO-d6) δ 11.19 (s, 1H), 7.41 (d, J=7.3 Hz, 2H), 7.35-7.16 (m, 8H), 6.92-6.77 (m, 4H), 5.61 (d, J=3.6 Hz, 1H), 5.41 (d, J=8.0 Hz, 1H), 4.79 (s, 1H), 4.29 (t, J=3.7 Hz, 1H), 4.02 (dd, J=9.0, 7.4 Hz, 1H), 3.84 (m, 1H), 3.71 (d, J=5.6 Hz, 6H), 3.17 (m, 2H), 2.85-2.67 (m, 1H), 2.45 (m, 1H).

Preparation of (5): A solution of compound 4 (980 mg, 1.85 mmol) in 2,2-dichloroacetic acid (20 mL, 3% in DCM) was stirred for 30 min at 0° C. under argon atmosphere. The resulting mixture was diluted with pyridine (2 mL). The resulting mixture was concentrated under reduced pressure, and resulted crude 5, which was used without purification.

Preparation of (6): A solution compound 5 and DMTrCl (1.47 g, 4.34 mmol) in pyridine (20 mL) was stirred for 2 h at room temperature under argon atmosphere. Reaction was quenched with water at room temperature. The resulting mixture was extracted with EtOAc (3×20 mL). The combined organic layers were washed with brine (2×30 mL), dried over anhydrous Na₂SO₄. After filtration, the filtrate was concentrated under reduced pressure. The residue was purified by reverse flash chromatography with the following conditions: column, C18; mobile phase, ACN in water, 10% to 90% gradient in 30 min; detector, UV 254 nm to give 6 (780 mg) as white solid.

Preparation of (7): A mixture of 1H-imidazole-4,5-dicarbonitrile (257.53 mg, 2.18 mmol) and CEP[N(iPr)2]2 (606.7 mg, 2.01 mmol) in DCM (8 mL) was stirred for 10 min at room temperature under argon atmosphere followed by the addition of compound 6 (890 mg, 1.68 mmol) dropwise/in portions at room temperature. The resulting mixture was stirred for 1 h at room temperature under argon atmosphere. The reaction was quenched with NaHCO₃(aqueous). The resulting mixture was extracted with CH₂C2 (3×10 mL). The combined organic layers were washed with brine (2×5 mL), dried over anhydrous Na₂SO₄. After filtration, the filtrate was concentrated under reduced pressure. The residue was purified by silica gel column chromatography, eluted with PE/EA with 0.5% TEA (1:1) to afford 7 (example 24 monomer) (950 mg, 75.56%) as a white solid. ESI-LCMS: m/z 731 [M+H]⁺; ¹HNMR: (300 MHz, DMSO-d6) δ 11.34 (d, J=8.4 Hz, 1H), 7.63 (t, J=7.9 Hz, 1H), 7.40-7.26 (m, 4H), 7.29-7.15 (m, 5H), 6.92-6.82 (m, 4H), 5.70 (d, J=5.0 Hz, 1H), 5.53 (d, J=8.1 Hz, 1H), 4.42 (s, 1H), 4.26 (q, J=8.2 Hz, 1H), 4.10-3.92 (m, 1H), 3.72 (d, J=1.6 Hz, 6H), 3.69-3.56 (m, 1H), 3.54-3.35 (m, 3H), 3.20-3.02 (m, 2H), 2.67 (q, J=7.3, 6.0 Hz, 2H), 1.04 (dd, J=6.7, 3.8 Hz, 6H), 0.92 (dd, J=18.1, 6.7 Hz, 6H); ³¹P NMR: (DMSO-d6) δ 149.57, 149.07

### Example 25: Synthesis of Monomer

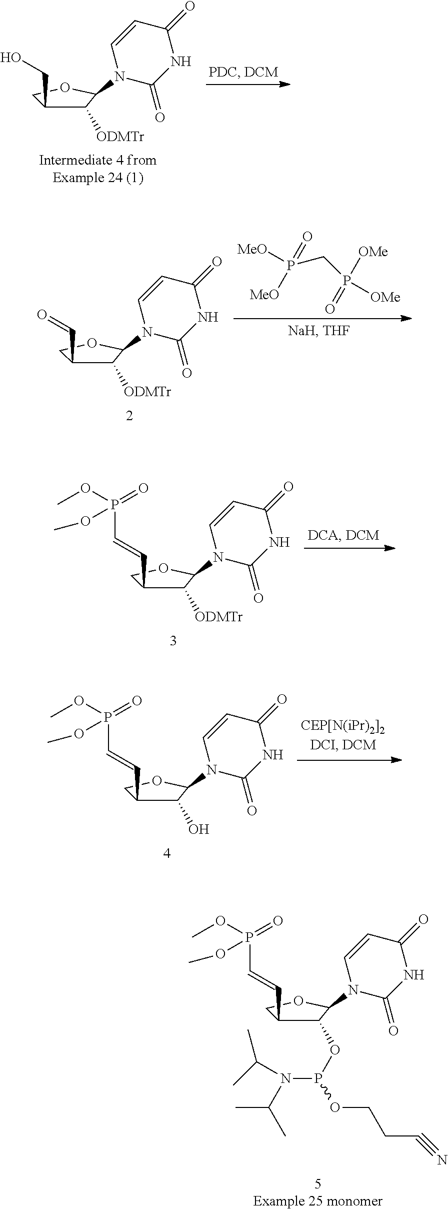

Preparation of (2): Into a 100 mL round-bottom flask were added compound 1 (intermediate 4, example 24) (1 g, 1.83 mmol), molecular sieve(1.7 g) and PDC (0.83 g, 2.2 mmol) at room temperature. To the above mixture was added DCM (30.00 mL). The resulting mixture was stirred for 2 h at room temperature under argon atmosphere. The precipitated solids were collected by filtration and washed with EtOAc (3×20 mL). The resulting mixture was concentrated under reduced pressure. This resulted in compound 2 (1.2 g, 124.10%) as a brown solid. The crude product 2 was used in the next step directly without further purification.

Preparation of (3): To a solution of dimethyl (dimethoxyphosphoryl)methylphosphonate (0.79 g, 3.4 mmol) in 18 mL THE was added sodium hydride (60%, 0.27 g) at −50° C. The mixture was stirred for 30 min. Compound 2 (1.2 g, 2.27 mmol) in 18 mL THE was added and the mixture was allowed to warm to room temperature and stirred for 1 h. The reaction was quenched with sat. NH₄Cl (aq.) at room temperature. The resulting mixture was extracted with EtOAc (3×30 mL). The combined organic layers were washed with brine (3×10 mL), dried over anhydrous Na₂SO₄. After filtration, the filtrate was concentrated under reduced pressure. The residue was purified by reverse flash chromatography with the following conditions: column, C18 silica gel; mobile phase, MeCN in water, 5% to 95% gradient in 30 min; detector, UV 254 nm. This resulted in 3 (680 mg, 47.20%) as a white solid. ESI-LCMS: m/z 633 [M+H]⁺; ¹H-NMR (400 MHz, Acetonitrile-d₃) δ 8.80 (s, 1H), 7.45-7.30 (m, 2H), 7.26-7.07 (m, 7H), 6.81-6.67 (m, 4H), 6.57 (d, J=8.0 Hz, 1H), 6.45 (ddd, J=21.7, 17.2, 8.5 Hz, 1H), 5.70 (dd, J=20.1, 17.3 Hz, 1H), 5.23 (d, J=8.0 Hz, 1H), 5.10 (d, J=3.1 Hz, 1H), 4.65 (dd, J=4.6, 3.1 Hz, 1H), 4.05 (t, J=8.4 Hz, 1H), 3.65 (d, J=5.4 Hz, 6H), 3.51 (dd, J=11.0, 6.1 Hz, 6H), 3.40-3.26 (m, 1H).

Preparation of (4): To a stirred solution of compound 3 (680 mg, 1.07 mmol) in DCM (20 mL) was added dichloroacetic acid (0.6 mL) dropwise at 0° C. under air atmosphere. The resulting mixture was stirred for 30 min at 0° C. under air atmosphere. The reaction was quenched with sat. NaHCO₃(aq.) at 0° C. The mixture was extracted with water (2×20 mL). The resulting mixture was concentrated to 25 mL under reduced pressure. The residue was purified by reverse flash chromatography with the following conditions: column, C18 silica gel; mobile phase, MeCN in water, 5% to 95% gradient in 115 min; detector, UV 254 nm. This resulted compound 4 (299 mg, 84%) as a white solid. ESI-LCMS: m/z 333 [M−H]⁻; ¹H NMR (400 MHz, Deuterium Oxide) δ 7.60 (d, J=8.1 Hz, 1H), 6.64 (ddd, J=22.6, 17.4, 7.2 Hz, 1H), 5.98-5.87 (m, 1H), 5.77 (d, J=8.1 Hz, 1H), 5.66 (d, J=4.7 Hz, 1H), 4.45 (dd, J=6.8, 4.7 Hz, 1H), 4.27 (dd, J=9.1, 7.8 Hz, 1H), 4.15 (dd, J=9.1, 8.0 Hz, 1H), 3.63 (d, J=11.2 Hz, 6H), 3.22 (qdd, J=7.0, 2.6, 1.3 Hz, 1H).

Preparation of (5): A solution of CEP[N(iPr)2]2 (272.16 mg, 0.9 mmol) in DCM (12 mL) was treated with molecular sieve under argon atmosphere followed by the addition of 1H-imidazole-4,5-dicarbonitrile (106.6 mg, 0.9 mmol). To the resulted solution, compound 4 (200 mg, 0.6 mmol) in 10 mL DCM was added dropwise slowly at room temperature. The resulting mixture was stirred for 1 h at room temperature under argon atmosphere. The resulting mixture was diluted with 0.5% TEA in DCM (20 mL). The reaction was quenched with Water at room temperature. The resulting mixture was extracted with 0.5% TEA in CH₂Cl₂ (3×15 mL). The combined organic layers were washed with brine (2×10 mL), dried over anhydrous MgSO₄. After filtration, the filtrate was concentrated under reduced pressure. The residue was purified by Prep-TLC (0.5% TEA in CH₂Cl₂/MeOH 12:1) to afford final monomer 5 (example 25) (170 mg, 50.60%) as off-white semi-solid. ESI-LCMS: m/z 533 [M+H]⁺; ¹H NMR (400 MHz, Acetonitrile-d3) δ 8.95 (s, 1H), 7.38 (dd, J=8.1, 6.1 Hz, 1H), 6.61 (dddd, J=21.5, 17.1, 11.1, 8.0 Hz, 1H), 5.92-5.64 (m, 2H), 5.56 (d, J=8.1 Hz, 1H), 4.64-4.44 (m, 1H), 4.11 (td, J=8.5, 4.5 Hz, 1H), 4.02 (td, J=8.9, 6.4 Hz, 1H), 3.82-3.36 (m, 10H), 3.24 (tq, J=16.6, 8.0 Hz, 1H), 2.59 (t, J=6.0 Hz, 1H), 2.54-2.45 (m, 1H), 1.09-0.97 (m, 12H); ³¹P NMR: δ 149.67, 149.32, 19.25, 19.13.

### Additional Tables

TABLE 17

| SEQ ID | | |
|---|---|---|
| NO: | Description | Sequence⁺ |
| 55 | Hepatitis B | aattccacaacctttcaccaaactctgcaagatcccagagtgagaggcctgtatttccctg |
| | virus | ctggtggctccagttcaggagcagtaaaccctgttccgactactgcctctcccttatcgtca |
| | (Genbank | atcttctcgaggattggggaccctgcgctgaacatggagaacatcacatcaggattccta |
| | Accession | ggaccccttctcgtgttacaggcggggtttttcttgttgacaagaatcctcacaataccgca |
| | No. | gagtctagactcgtggtggacttctctcaattttctagggggaactaccgtgtgtcttggcc |
| | U95551.1) | aaaattcgcagtccccaacctccaatcactcaccaacctcctgtcctccaacttgtcctggt |
| | | tatcgctggatgtgtctgcggcgttttatcatcttcctcttcatcctgctgctatgcctcatcttc |
| | | ttgttggttcttctggactatcaaggtatgttgcccgtttgtcctctaattccaggatcctcaac |
| | | caccagcacgggaccatgccgaacctgcatgactactgctcaaggaacctctatgtatcc |
| | | ctcctgttgctgtaccaaaccttcggacggaaattgcacctgtattcccatcccatcatcct |
| | | gggctttcggaaaattcctatgggagtgggcctcagcccgtttctcctggctcagtttacta |
| | | gtgccatttgttcagtggttcgtagggctttcccccactgtttggctttcagttatatggatgat |
| | | gtggtattgggggccaagtctgtacagcatcttgagtccctttttaccgctgttaccaattttc |
| | | ttttgtctttgggtatacatttaaaccctaacaaaacaaagagatggggttactctctgaatttt |
| | | atgggttatgtcattggaagttatgggtccttgccacaagaacacatcatacaaaaaatca |
| | | aagaatgttttagaaaacttcctattaacaggcctattgattggaaagtatgtcaacgaattg |
| | | tgggtcttttgggttttgctgccccatttacacaatgtggttatcctgcgttaatgcccttgtat |
| | | gcatgtattcaatctaagcaggctttcactttctcgccaacttacaaggcctttctgtgtaaac |
| | | aatacctgaacctttaccccgttgcccggcaacggccaggtctgtgccaagtgtttgctga |
| | | cgcaacccccactggctggggcttggtcatgggccatcagcgcgtgcgtggaacctttt |
| | | cggctcctctgccgatccatactgcggaactcctagccgcttgttttgctcgcagcaggtc |
| | | tggagcaaacattatcgggactgataactctgttgtcctctcccgcaaatatacatcgtatc |
| | | catggctgctaggctgtgctgccaactggatcctgcgcgggacgtcctttgtttacgtccc |
| | | gtcggcgctgaatcctgcggacgacccttctcggggtcgcttgggactctctcgtcccctt |
| | | ctccgtctgccgttccgaccgaccacggggcgcacctctctttacgcggactccccgtct |
| | | gtgccttctcatctgccggaccgtgtgcacttcgcttcacctctgcacgtcgcatggagac |
| | | caccgtgaacgcccaccgaatgttgcccaaggtcttacataagaggactcttggactctct |
| | | gcaatgtcaacgaccgaccttgaggcatacttcaaagactgtttgtttaaagactgggagg |
| | | agttgggggaggagattagattaaaggtctttgtactaggaggctgtaggcataaattggt |
| | | ctgcgcaccagcaccatgcaactttttcacctctgcctaatcatctcttgttcatgtcctactg |
| | | ttcaagcctccaagctgtgccttgggtggctttggggcatggacatcgacccttataaaga |
| | | atttggagctactgtggagttactctcgtttttgccttctgacttctttccttcagtacgagatct |
| | | tctagataccgcctcagctctgtatcgggaagccttagagtctcctgagcattgttcacctc |
| | | accatactgcactcaggcaagcaattctttgctggggggaactaatgactctagctacctg |
| | | ggtgggtgttaatttggaagatccagcatctagagacctagtagtcagttatgtcaacacta |
| | | atatgggcctaaagttcaggcaactcttgtggtttcacatttcttgtctcacttttggaagaga |
| | | aaccgttatagagtatttggtgtctttcggagtgtggattcgcactcctccagcttatagacc |
| | | accaaatgcccctatcctatcaacacttccggaaactactgttgttagacgacgaggcag |
| | | gtcccctagaagaagaactccctcgcctcgcagacgaaggtctcaatcgccgcgtcgca |
| | | gaagatctcaatctcgggaacctcaatgttagtattccttggactcataaggtggggaactt |
| | | tactggtctttattcttctactgtacctgtctttaatcctcattggaaaacaccatcttttcctaat |
| | | atacatttacaccaagacattatcaaaaaatgtgaacagtttgtaggcccacttacagttaat |
| | | gagaaaagaagattgcaattgattatgcctgctaggttttatccaaaggttaccaaatattta |
| | | ccattggataagggtattaaaccttattatccagaacatctagttaatcattacttccaaacta |
| | | gacactatttacacactctatggaaggcgggtatattatataagagagaaacaacacatag |
| | | cgcctcattttgtgggtcaccatattcttgggaacaagatctacagcatggggcagaatctt |
| | | tccaccagcaatcctctgggattctttcccgaccaccagttggatccagccttcagagcaa |
| | | acacagcaaatccagattgggacttcaatcccaacaaggacacctggccagacgccaa |
| | | caaggtaggagctggagcattcgggctgggtttcaccccaccgcacggaggccttttgg |
| | | ggtggagccctcaggctcagggcatactacaaactttgccagcaaatccgcctcctgcct |
| | | ccaccaatcgccagacaggaaggcagcctaccccgctgtctccacctttgagaaacact |
| | | catcctcaggccatgcagtgg |
| 56 | MCJ mRNA | agtcactgccgcggcgccttgagtctccgggccgccttgccatggctgcccgtggtgtcatc |
| | (GenBank | gctccagttggcgagagtttgcgctacgctgagtacttgcagccctcggccaaacggccag |
| | Accession | acgccgacgtcgaccagcagagactggtaagaagtttgatagctgtaggactgggtgttgc |
| | No. | agctcttgcatttgcaggtcgctacgcatttcggatctggaaacctctagaacaagttatcaca |
| | NM_013238.3) | gaaactgcaaagaagatttcaactcctagcttttcatcctactataaaggaggatttgaacaga |
| | | aaatgagtaggcgagaagctggtcttattttaggtgtaagcccatctgctggcaaggctaaga |
| | | ttagaacagctcataggagagtcatgattttgaatcacccagataaaggtggatctccttacgt |
| | | agcagccaaaataaatgaagcaaaagacttgctagaaacaaccaccaaacattgatgcttaa |
| | | ggaccacactgaaggaaaaaaaaagaggggacttcgaaaaaaaaaaaagccctgcaaaa |
| | | tattctaaaacatggtcttcttaattttctatatggattgaccacagtcttatcttccaccattaagct |
| | | gtataacaataaaatgttaatagtcttgctttttattatcttttaaagatctccttaaattctataactg |
| | | atcttttttcttattttgtttgtgacattcatacatttttaagatttttgttatgttctgaattcccccctac |
| | | acacacacacacacacacacacacacacacgtgcaaaaaatatgatcaagaatgcaattgg |
| | | gatttgtgagcaatgagtagacctcttattgtttatatttgtaccctcattgtcaatttttttttaggg |
| | | aatttgggactctgcctatataaggtgttttaaatgtcttgagaacaagcactggctgatacctc |
| | | ttggagatatgatctgaaatgtaatggaatttattaaatggtgtttagtaaagtaggggttaagg |
| | | acttgttaaagaaccccactatctctgagaccctatagccaaagcatgaggacttggagagct |
| | | actaaaatgattcaggtttacaaaatgagccctgtgaggaaaggttgagagaagtctgagga |
| | | gtttgtatttaattatagtcttccagtactgtatattcattcattactcattctacaaatatttattgacc |
| | | ccttttgatgtgcaaggcactatcgtgcgtcccctgagagttgcaagtatgaagcagtcatgg |
| | | atcatgaaccaaaggaacttatatgtagaggaaggataaatcacaaatagtgaatactgttag |
| | | atacagatgatatattttaaaagttcaaaggaagaaaagaatgtgttaaacactgcatgagag |
| | | gaggaataagtggcatagagctaggctttagaaaagaaaaatattccgataccatatgattgg |
| | | tgaggtaagtgttattctgagatgagaattagcagaaatagatatatcaatcggagtgattaga |
| | | gtgcagggtttctggaaagcaaggtttggacagagtggtcatcaaaggccagccctgtgact |
| | | tacactgcattaaattaatttcttagaacatagtccctgatcattatcactttactattccaaaggt |
| | | gagagaacagattcagatagagtgccagcattgtttcccagtattcctttacaaatcttgggttc |
| | | attccaggtaaactgaactactgcattgtttctatcttaaaatactttttagatatcctagatgcatc |
| | | tttcaacttctaacattctgtagtttaggagttctcaaccttggcattattgacatgttaggccaaa |
| | | taattttttttgtgggaggtctcttgtgcgttttagatgattagcaataatccctgacctgttatcta |
| | | ctaaagactagtcgtttctcatcagttgtgacaacaaaaatggttccagatattgccaaatgcc |
| | | ctttagaggacagtaatcgcccccagttgagaaccatttcagtaaaactttaattactattttttct |
| | | tttggtttataaaataatgatcctgaattaaattgatggaaccttgaagtcgataaaatatatttctt |
| | | gctttaaagtccccatacgtgtcctactaattttctcatgctttagtgttttcacttttctcctgttatc |
| | | cttgtacctaagaatgccatcccaatccccagatgtccacctgcccaaagtctaggcatagct |
| | | gaaggccaagctaaaatgtatccctctttttctggtacatgcagcaaaagtaatatgaattatca |
| | | gctttctgagagcaggcattgtatctgtcttgtttggtgttacattggcacccaataaatatttgtt |
| | | gagtgaatgaataaattcccatagcactttattcttcacatggtacataactataggggctatag |
| | | cttggtaccttgtgaagcaactcttggtgtaacataccttatttctcatactaaaatgcaagaacc |
| | | tttagagcaaggatcttgccattcatctttgtaacctctttactctggagcacttgcatttagcag |
| | | gcatcataaagttttacgtaccaagaaaatgttgctgttttctgaatactatgcatcaaaaaatgt |
| | | taccactaatttttaaagctctgctaaggaatattggggcaccctcagatgcaccttttaattgat |
| | | gtcatattttcctaatccatactttattcatgagaatttgagtcaccccagcattagcttggaatttc |
| | | cttatttcccatttgctttgcaggtgccttggagtcagatctggttttgaatactatcttcctgttat |
| | | gtgatcttgggcagttacttaattttctagtcaataacccgtatctataaaatagagaaaataatc |
| | | ctacacaccggggcctgttgtggggcggggagaggggggagggatcgcatttggagatat |
| | | actaatgtaaatgacaagttaattggtgcagcacaccaacatggctcatgtctacatatgtaac |
| | | aaacctgcacgttgtgcacatgtgccctagaacttaaagtataataaaaagaaattttaaaaaa |
| | | tcctgtcaaataaggttatagtagagaataaggatgtgtaaagcatttagtcacgtaaatgctta |
| | | aaaaaatgtaatttttacttctttcactgcctcatttaattagttttatctttaataataccttggattca |
| | | gggtaaagtttcagttatgtcccagtaatcatttattttaccctcgaatctgcaatttggatagaa |
| | | catggtggggacagctcgtctctattccttgcagcattaacaggctggaggcaccacttctct |
| | | ggccagcaagttgggcctggttgttggctgagagcctcagttcctttctgcacaggttcctctt |
| | | tacataggcttctcaacagggctactagagcatcgtcaccatagcagctgtcttataacagag |
| | | agtggtcggtctgagagacaaaaaatggaagctgccaaattgttctgggtctggaaactgtc |
| | | agggcatcacttgtgccatattcagttggcctaagaattacagagcctgcctcgattcaaagg |
| | | gagaggatagagaggactgaaggaatcagtgctcatctttaatatgcagcaggacaggtttg |
| | | ggattttttttcccccttgagtctgtgaaggcattacttaagaacaaagtcaggcatgtataattg |
| | | aactacagttacttgaaatataagcccagaaagtttcagataataaatacaactatttttctgctg |
| | | ttacccttgtacctaaagatgccatcctaatccccagatctccacaactatacctacatagtaga |
| | | aggttaaaatgtatccctctttttctggtgcatccagcaaaagtaatatcatgaattatgagctct |
| | | ctgagagcaaggatcatatcagtcttgtttattgttgcagtgaacaagtacagttgcagatattc |
| | | aggagtaattatctaaatggcagtaggcttataaaactgaattttcaccagccacaccctcccc |
| | | ccaactccttatctgtaaaaagcttatttgagtggttacctgtcttcagtaaagattgcgcttgca |
| | | tatttgctgtcattgcatattctgcttaattaagctctgttgatattgcagtttctgtgcatacttacat |
| | | cttagatgcaatctgagggcctaggaaggccttttaaaaataaaacaattccgattgcagaga |
| | | aagtgtaagtcaaggacagttaattcaaggggaacatagaaagctatttagattttagttgatg |
| | | gtgccagtcttcagcgtaaagtcaaaagtggagggaagtttagtaaggaaaaaatgttgggc |
| | | ttggaatacattgtttagtcttcaaagcactttactttttatgaaatatattttagacattcagcaaat |
| | | attgaatacttactatatcaggcagtaaagatataaattcattcttaaaatgtgcaacatgttcaa |
| | | actgaaaaaaatacattcttaaacaggaaactttttccttcatactttttaattaacaagacatata |
| | | agagttgcattaatgggcgtgcttatgattgatcacccagcagcatcattagaaataatatatttt |
| | | attcatgtgcagaaatcttttggttgtcctggggaaccttgaacacagaaaagagcttttattga |
| | | taaggtaattgaacacacttgacaattagcttaatatggtttaataccatttgtgggagaagatg |
| | | aatcagccaggctctttacgtcaagaatatgaagtttctcttgagtcaaccaacttaagatgag |
| | | ctacggagactgcagtgaaaagttaaatatccaagtacaccagccaatttcacacagtggaa |
| | | ccatgctgtcctcgggcaccctgcacctcgcccaacagtcatcaactagatggaggctcctg |
| | | gctgcaaggaggatttgatgggaatgagtaaatgtgtcagcatagtccgtcccttctaatgga |
| | | aaagcaacccaaagagcaaatcctattaatggctggatcagtatcatctacttgtcaaaaaca |
| | | ttccatgaattatgagtcaaaattttatttatggtggcattacacacattaagagatgaggacttc |
| | | tgttagcataatttattagctggaaaagttgagaaggttctctggactcatttttataggtggaac |
| | | ctaagtgatctggataattgcccaccagcaaaattgctgggcatggtggacaaagaaaatgtt |
| | | ccttctaatgattttttatgagctgagtagctattgttcccagctgagtgctcttttcctctttttattg |
| | | ttgctgagcaaaagaatttataaaaagctctttcttttgtattaaaaaccctgctcaattgaaatg |
| | | caagttcattaagtaatcttcatttctcttcctgccataataaccctttccctctctgttcgattcaa |
| | | cagtatctagcagcactgctccaaattttaagtctgaacagactatattacatagatgtagaga |
| | | aatactcaatcttcagcattaagagggagcttaatttcacacgggtggaatatgatcactcagg |
| | | ctagatgttggccataaatttcaaattagtatctcaacttagcaggggggatcaacagtggca |
| | | aacttcaattatgacaggataaaaatcacatagagatattggttcaatatggacatctaaactat |
| | | aatgctaaaagccaataattagaataagttcattttaagaaaagcattaataatattagctaacg |
| | | tttagtacctgtgccaaacattctacctatgttaccttgattttcatagccagcctaagaggtact |
| | | attatgtatccccattttacaggttaagaaacaggctcagaggagtttaggatcttttccaagatt |
| | | acatagccagtaagtggtggcactaggaaccaaattcagactctgaatcgcatgctgtttatat |
| | | tatattgcactcattctaaatatgtgggaatcagaatgaaggggcttgtatgacttttggctcatt |
| | | ttttgatgcatgtgacctgggattataaatgtgaaattaggtttacgaaaggatccagtgtcatt |
| | | gtgcatcatgggcaaggagtacctaatctctttaattcttccctggaagcttacgatgtccatcc |
| | | aagtgcacatagcaaaagttctgttgtaaagtttagcagagtgactttctttgactcagagtgat |
| | | gacggaggaagctttgataagattttatctgaaatgttcatggacaagagctttcaaggagaa |
| | | catccagagcaaggttctgaagacagctcatgaaggtgaagcagcagacctggcacaaga |
| | | aatgaagagagagctcagtgtattaaagatgaaaacaagaaaaccgaatatattgaaagga |
| | | gcagagaggcaatgaaaacaagacaactgaaatgaggtaacttgcagcaattgaaaggga |
| | | atttcagtacttttatagaattcttaaaaattgtttcctgctgtttattttcaattttgaacagggttatt |
| | | tgtccatgccatactttttttgccaaattccaaaattgtgtatagttctatagttgtctggtggagtc |
| | | aatggaactttagttaccagtctaagaatgtgtctttgagattgtccagttaattctctatttccagt |
| | | agctgtaataaatggtgaaaaggtttctgactcctggagaaagtttctaactccttatgactaat |
| | | attcataacagacttgtgagttccttgaacatggatacacctatatgcaagagtgtattccaaag |
| | | ctaactcagtgatctttccatttatctattcttggattagtggtgcctttgctctttccttctgtaaatg |
| | | tgaatagttaagagttgactgcagaagtgtttacactttggcttccatgcctctggaatgtttgtg |
| | | ctttggtggtgagatgtgagactatatttgtatagtctgcatctctcaggctgccccagaatgtt |
| | | gtacagtgcagtgctgaagaaagcagcaggtacacacagaaatgcagcctttcctggttaa |
| | | ccctgcttggatctgagttacactttgtttcctgacttcttgggacttaggtaatcagtttgccttct |
| | | actctatctcattttgtactcgcttacatactacattcttgtttgggctttcgtttcttcttgtaagcag |
| | | agattttttaaaatccaatatgtgaaaatacggatgcactacaattaaataaataaaatgctgttg |
| | | tgtttgttttgctttaaaattgtaaaggataaacaataagatagttttatctatgtggttttcccgatg |
| | | cagttaaaataaaacctaatctgctaaaattgaa |
| 57 | TAZ | gctttccggcggttgcaccgggccggggtgccagcgcccgccttcccgtttcctcccgttcc |
| | (GenBank | gcagcgcgcccacggcctgtgaccccggcgaccgctccccagtgacgagagagcgggg |
| | Accession | ccgggcgctgctccggcctgacctgcgaagggacctcggtccagtcccctgttgcgccgc |
| | No. | gcccccgtccgtccgtgcgcgggccagtcaggggccagtgtctcgagcggtcgaggtcg |
| | NM_000116.5) | cagacctagaggcgccccacaggccggcccggggcgctgggagcgccggccgcgggc |
| | | cgggtggggatgcctctgcacgtgaagtggccgttccccgcggtgccgccgctcacctgg |
| | | accctggccagcagcgtcgtcatgggcttggtgggcacctacagctgcttctggaccaagt |
| | | acatgaaccacctgaccgtgcacaacagggaggtgctgtacgagctcatcgagaagcgag |
| | | gcccggccacgcccctcatcaccgtgtccaatcaccagtcctgcatggacgaccctcatctc |
| | | tgggggatcctgaaactccgccacatctggaacctgaagttgatgcgttggacccctgcagc |
| | | tgcagacatctgcttcaccaaggagctacactcccacttcttcagcttgggcaagtgtgtgcct |
| | | gtgtgccgaggagcagaatttttccaagcagagaatgaggggaaaggtgttctagacacag |
| | | gcaggcacatgccaggtgctggaaaaagaagagagaaaggagatggcgtctaccagaag |
| | | gggatggacttcattttggagaagctcaaccatggggactgggtgcatatcttcccagaagg |
| | | gaaagtgaacatgagttccgaattcctgcgtttcaagtggggaatcgggcgcctgattgctg |
| | | agtgtcatctcaaccccatcatcctgcccctgtggcatgtcggaatgaatgacgtccttcctaa |
| | | cagtccgccctacttcccccgctttggacagaaaatcactgtgctgatcgggaagcccttca |
| | | gtgccctgcctgtactcgagcggctccgggcggagaacaagtcggctgtggagatgcgga |
| | | aagccctgacggacttcattcaagaggaattccagcatctgaagactcaggcagagcagct |
| | | ccacaaccacctccagcctgggagataggccttgcttgctgccttctggattcttggcccgca |
| | | cagagctggggctgagggatggactgatgcttttagctcaaacgtggcttttagacagatttg |
| | | ttcatagaccctctcaagtgccctctccgagctggtaggcattccagctcctccgtgcttcctc |
| | | agttacacaaaggacctcagctgcttctcccacttggccaagcagggaggaagaagcttag |
| | | gcagggctctctttccttcttgccttcagatgttctctcccaggggctggcttcaggagggagc |
| | | atagaaggcaggtgagcaaccagttggctaggggagcagggggcccaccagagctgtg |
| | | gagaggggaccctaagactcctcggcctggctcctacccaccgcccttgccgaaccagga |
| | | gctgctcactacctcctcagggatggccgttggccacgtcttccttctgcctgagcttcccccc |
| | | caccacaggccctttcctcaggcaaggtctggcctcaggtgggccgcaggcgggaaaag |
| | | cagcccttggccagaagtcaagcccagccacgtggagcctagagtgagggcctgaggtct |
| | | ggctgcttgcccccatgctggcgccaacaacttctccatcctttctgcctctcaacatcacttg |
| | | aatcctagggcctgggttttcatgtttttgaaacagaaccataaagcatatgtgttggcttgttgt |
| | | aaaa |
| 58 | ANGPTL3 | agaagaaaacagttccacgttgcttgaaattgaaaatcaagataaaaatgttcacaattaagct |
| | (GenBank | ccttctttttattgttcctctagttatttcctccagaattgatcaagacaattcatcatttgattctctat |
| | Accession | ctccagagccaaaatcaagatttgctatgttagacgatgtaaaaattttagccaatggcctcctt |
| | No. | cagttgggacatggtcttaaagactttgtccataagacgaagggccaaattaatgacatatttc |
| | NM_014495.4) | aaaaactcaacatatttgatcagtctttttatgatctatcgctgcaaaccagtgaaatcaaagaa |
| | | gaagaaaaggaactgagaagaactacatataaactacaagtcaaaaatgaagaggtaaag |
| | | aatatgtcacttgaactcaactcaaaacttgaaagcctcctagaagaaaaaattctacttcaac |
| | | aaaaagtgaaatatttagaagagcaactaactaacttaattcaaaatcaacctgaaactccag |
| | | aacacccagaagtaacttcacttaaaacttttgtagaaaaacaagataatagcatcaaagacc |
| | | ttctccagaccgtggaagaccaatataaacaattaaaccaacagcatagtcaaataaaagaa |
| | | atagaaaatcagctcagaaggactagtattcaagaacccacagaaatttctctatcttccaag |
| | | ccaagagcaccaagaactactccctttcttcagttgaatgaaataagaaatgtaaaacatgat |
| | | ggcattcctgctgaatgtaccaccatttataacagaggtgaacatacaagtggcatgtatgcc |
| | | atcagacccagcaactctcaagtttttcatgtctactgtgatgttatatcaggtagtccatggac |
| | | attaattcaacatcgaatagatggatcacaaaacttcaatgaaacgtgggagaactacaaata |
| | | tggttttgggaggcttgatggagaattttggttgggcctagagaagatatactccatagtgaag |
| | | caatctaattatgttttacgaattgagttggaagactggaaagacaacaaacattatattgaata |
| | | ttctttttacttgggaaatcacgaaaccaactatacgctacatctagttgcgattactggcaatgt |
| | | ccccaatgcaatcccggaaaacaaagatttggtgttttctacttgggatcacaaagcaaaagg |
| | | acacttcaactgtccagagggttattcaggaggctggtggtggcatgatgagtgtggagaaa |
| | | acaacctaaatggtaaatataacaaaccaagagcaaaatctaagccagagaggagaagag |
| | | gattatcttggaagtctcaaaatggaaggttatactctataaaatcaaccaaaatgttgatccat |
| | | ccaacagattcagaaagctttgaatgaactgaggcaaatttaaaaggcaataatttaaacatta |
| | | acctcattccaagttaatgtggtctaataatctggtattaaatccttaagagaaagcttgagaaa |
| | | tagattttttttatcttaaagtcactgtctatttaagattaaacatacaatcacataaccttaaagaa |
| | | taccgtttacatttctcaatcaaaattcttataatactatttgttttaaattttgtgatgtgggaatcaa |
| | | ttttagatggtcacaatctagattataatcaataggtgaacttattaaataacttttctaaataaaa |
| | | aatttagagacttttattttaaaaggcatcatatgagctaatatcacaactttcccagtttaaaaaa |
| | | ctagtactcttgttaaaactctaaacttgactaaatacagaggactggtaattgtacagttcttaa |
| | | atgttgtagtattaatttcaaaactaaaaatcgtcagcacagagtatgtgtaaaaatctgtaatac |
| | | aaatttttaaactgatgcttcattttgctacaaaataatttggagtaaatgtttgatatgatttatttat |
| | | gaaacctaatgaagcagaattaaatactgtattaaaataagttcgctgtctttaaacaaatgga |
| | | gatgactactaagtcacattgactttaacatgaggtatcactataccttatttgttaaaatatatac |
| | | tgtatacattttatatattttaacacttaatactatgaaaacaaataattgtaaaggaatcttgtcag |
| | | attacagtaagaatgaacatatttgtggcatcgagttaaagtttatatttcccctaaatatgctgt |
| | | gattctaatacattcgtgtaggttttcaagtagaaataaacctcgtaacaagttactgaacgttta |
| | | aacagcctgacaagcatgtatatatgtttaaaattcaataaacaaagacccagtccctaaatta |
| | | tagaaatttaaattattcttgcatgtttatcgacatcacaacagatccctaaatccctaaatcccta |
| | | aagattagatacaaattttttaccacagtatcacttgtcagaatttatttttaaatatgattttttaaa |
| | | actgccagtaagaaattttaaattaaacccatttgttaaaggatatagtgcccaagttatatggt |
| | | gacctacctttgtcaatacttagcattatgtatttcaaattatccaatatacatgtcatatatattttta |
| | | tatgtcacatatataaaagatatgtatgatctatgtgaatcctaagtaaatattttgttccagaaaa |
| | | gtacaaaataataaaggtaaaaataatctataattttcaggaccacagactaagctgtcgaaat |
| | | taacgctgatttttttagggccagaataccaaaatggctcctctcttcccccaaaattggacaat |
| | | ttcaaatgcaaaataattcattatttaatatatgagttgcttcctctatttggtttcc |
| 59 | DGAT2 | tgccccgttgtgaggtgataaagtgttgcgctccgggacgccagcgccgcggctgccgcct |
| | (GenBank | ctgctggggtctaggctgtttctctcgcgccaccactggccgccggccgcagctccaggtgt |
| | Accession | cctagccgcccagcctcgacgccgtcccgggacccctgtgctctgcgcgaagccctggcc |
| | No. | ccgggggccggggcatgggccaggggcgcggggtgaagcggcttcccgcggggccgt |
| | NM_001253891.1) | gactgggcgggcttcagccatgaagaccctcatagccgcctactccggggtcctgcgcgg |
| | | cgagcgtcaggccgaggctgaccggagccagcgctctcacggaggacctgcgctgtcgc |
| | | gcgaggggtctgggagatggggagtggcctgcagtgccatcctcatgtacatattctgcact |
| | | gattgctggctcatcgctgtgctctacttcacttggctggtgtttgactggaacacacccaaga |
| | | aaggtggcaggaggtcacagtgggtccgaaactgggctgtgtggcgctactttcgagacta |
| | | ctttcccatccagctggtgaagacacacaacctgctgaccaccaggaactatatctttggata |
| | | ccacccccatggtatcatgggcctgggtgccttctgcaacttcagcacagaggccacagaa |
| | | gtgagcaagaagttcccaggcatacggccttacctggctacactggcaggcaacttccgaa |
| | | tgcctgtgttgagggagtacctgatgtctggaggtatctgccctgtcagccgggacaccata |
| | | gactatttgctttcaaagaatgggagtggcaatgctatcatcatcgtggtcgggggtgcggct |
| | | gagtctctgagctccatgcctggcaagaatgcagtcaccctgcggaaccgcaagggctttgt |
| | | gaaactggccctgcgtcatggagctgacctggttcccatctactcctttggagagaatgaagt |
| | | gtacaagcaggtgatcttcgaggagggctcctggggccgatgggtccagaagaagttcca |
| | | gaaatacattggtttcgccccatgcatcttccatggtcgaggcctcttctcctccgacacctgg |
| | | gggctggtgccctactccaagcccatcaccactgttgtgggagagcccatcaccatcccca |
| | | agctggagcacccaacccagcaagacatcgacctgtaccacaccatgtacatggaggccc |
| | | tggtgaagctcttcgacaagcacaagaccaagttcggcctcccggagactgaggtcctgga |
| | | ggtgaactgagccagccttcggggccaattccctggaggaaccagctgcaaatcactttttt |
| | | gctctgtaaatttggaagtgtcatgggtgtctgtgggttatttaaaagaaattataacaattttgct |
| | | aaaccattacaatgttaggtcttttttaagaaggaaaaagtcagtatttcaagttctttcacttcca |
| | | gcttgccctgttctaggtggtggctaaatctgggcctaatctgggtggctcagctaacctctctt |
| | | cttcccttcctgaagtgacaaaggaaactcagtcttcttggggaagaaggattgccattagtg |
| | | acttggaccagttagatgattcactttttgcccctagggatgagaggcgaaagccacttctcat |
| | | acaagcccctttattgccactaccccacgctcgtctagtcctgaaactgcaggaccagtttctc |
| | | tgccaaggggaggagttggagagcacagttgccccgttgtgtgagggcagtagtaggcat |
| | | ctggaatgctccagtttgatctcccttctgccacccctacctcacccctagtcactcatatcgga |
| | | gcctggactggcctccaggatgaggatgggggtggcaatgacaccctgcaggggaaagg |
| | | actgccccccatgcaccattgcagggaggatgccgccaccatgagctaggtggagtaact |
| | | ggtttttcttgggtggctgatgacatggatgcagcacagactcagccttggcctggagcacat |
| | | gcttactggtggcctcagtttaccttccccagatcctagattctggatgtgaggaagagatccc |
| | | tcttcagaaggggcctggccttctgagcagcagattagttccaaagcaggtggcccccgaa |
| | | cccaagcctcacttttctgtgccttcctgagggggttgggccggggaggaaacccaaccctc |
| | | tcctgtgtgttctgttatctcttgatgagatcattgcaccatgtcagacttttgtatatgccttgaaa |
| | | ataaatgaaagtgagaatcctctaaaaaaaaaaaa |
| 60 | HBV | ctccaccactttccaccaaactcttcaagatcccagagtcagggccctgtactttcctgctggt |
| | Genbank | ggctcaagttccggaacagtaaaccctgctccgactactgcctctcccatatcgtcaatcttct |
| | Accession | cgaggactggggaccctgtaccgaatatggagagcaccacatcaggattcctaggacccct |
| | No. | gctcgtgttacaggcggggtttttcttgttgacaagaatcctcacaataccacagagtctagac |
| | KC315400.1 | tcgtggtggacttctctcaattttctagggggagcacccacgtgtcctggccaaaatttgcagt |
| | | ccccaacctccaatcactcaccaacctcttgtcctccaatttgtcctggttatcgctggatgtgt |
| | | ctgcggcgttttatcatcttcctcttcatcctgctgctatgcctcatcttcttgttggttcttctggac |
| | | taccaaggtatgttgcccgtttgtcctctacttccaggaacatcaactaccagcaccggaccat |
| | | gcaaaacctgcacaactactgctcaagggacctctatgtttccctcatgttgctgtacaaaacc |
| | | tacggacggaaactgcacctgtattcccatcccatcatcttgggctttcgcaaaatacctatgg |
| | | gagtgggcctcagtccgtttctcttggctcagtttactagtgccatttgttcagtggttcgtagg |
| | | gctttcccccactgtctggctttcagttatatggatgatgtggttttgggggccaagtctgtaca |
| | | acatcttgagtccctttataccgctgttaccaattttcttttatctttgggtatacatttaaaccctca |
| | | caaaacaaaaagatggggatattcccttaacttcatgggatatgtaattgggagttggggcac |
| | | tttgcctcaggaacatattgtacaaaaaatcaagcaatgttttaggaaacttcctgtaaacagg |
| | | cctattgattggaaagtatgtcaacraattgtgggtcttttggggtttgccgcccctttcacgca |
| | | atgtggatatcctgctttaatgcctttatatgcatgtatacaagctaagcaggcttttactttctcg |
| | | ccaacttacaaggcctttctgtgtaaacaatatctgaacctttaccccgttgctcggcaacggt |
| | | caggtctttgccaagtgtttgctgacgcaacccccactggttggggcttggccataggccatc |
| | | agcgcatgcgtggaacctttgtggctcctctgccgatccatactgcggaactcctagcagctt |
| | | gttttgctcgcagccggtctggagcaaaacttatcggcaccgacaactctgttgtcctctctcg |
| | | gaaatacacctcctttccatggctgctaggatgtgctgccaactggatcctgcgcgggacgt |
| | | cctttgtctacgtcccgtcggcgctgaatcccgcggacgacccatctcggggccgtttggga |
| | | ctctaccgtccccttctgcgtctgccgttccgcccgaccacggggcgcacctctctttacgcg |
| | | gtctccccgtctgtgccttctcatctgccggaccgtgtgcacttcgcttcacctctgcacgtcg |
| | | catggagaccaccgtgaacgcccacgggaacctgcccaaggtcttgcataagaggactctt |
| | | ggactttcagcaatgtcaacgaccgaccttgaggcatacttcaaagactgtgtgtttactgagt |
| | | gggaggagttgggggaggaggttaggttaaaggtctttgtactaggaggctgtaggcataa |
| | | attggtgtgttcaccagcaccatgcaactttttcacctctgcctaatcatctcatgttcatgtccta |
| | | ctgttcaagcctccaagctgtgccttgggtggctttggggcatggacattgacccgtataaag |
| | | aatttggagcttctgtggagttactctcttttttgccttctgacttctttccttctattcgagatctcct |
| | | cgacaccgcctctgctctgtatcgggaggccttagagtctccggaacattgttcacctcacca |
| | | tacggcactcaggcaagcaattctgtgttggggtgagttaatgaatctagccacctgggtgg |
| | | gaagtaatttggaagatccagcatccagggaattagtagtcagctatgtcaacgttaatatgg |
| | | gcctaaaaatcagacaactattgtggtttcacatttcctgtcttacttttgggagagaaactgttc |
| | | ttgaatatttggtgtcttttggagtgtggattcgcactcctcctgcatatagaccacaaaatgcc |
| | | cctatcttatcaacacttccggaaactactgttgttagacgaagaggcaggtcccctagaaga |
| | | agaactccctcgcctcgcagacgaaggtctcaatcgccgcgtcgcagaagatctcaatctc |
| | | gggaatctcaatgttagtattccttggacacataaggtgggaaactttacggggctttattcttct |
| | | acggtaccttgctttaatcctaaatggcaaactccttcttttcctgacattcatttgcaggaggac |
| | | attgttgatagatgtaagcaatttgtggggccccttacagtaaatgaaaacaggagacttaaat |
| | | taattatgcctgctaggttttatcccaatgttactaaatatttgcccttagataaagggatcaaac |
| | | cgtattatccagagtatgtagttaatcattacttccagacgcgacattatttacacactctttgga |
| | | aggcggggatcttatataaaagagagtccacacgtagcgcctcattttgcgggtcaccatatt |
| | | cttgggaacaagatctacagcatgggaggttggtcttccaaacctcgaaaaggcatgggga |
| | | caaatctttctgtccccaatcccctgggattcttccccgatcatcagttggaccctgcattcaaa |
| | | gccaactcagaaaatccagattgggacctcaacccacacaaggacaactggccggacgcc |
| | | aacaaggtgggagtgggagcattcgggccagggttcacccctcctcatgggggactgttg |
| | | gggtggagccctcaggctcagggcatattcacaacagtgccagcagctcctcctcctgcct |
| | | ccaccaatcggcagtcaggaaggcagcctactcccttctctccacctctaagagacactcat |
| | | cctcaggccatgcagtggaa |
| 61 | ASO 1 | GalNAc4-ps-GalNAc4-ps-GalNAc4-po-mA-po- |
| | | lnGpslnApslnTpslnApslnApsApsAps(5OH)CpsGps(5m)Cps(5m) |
| | | CpsGps(5m)CpslnApslnGpslnApscp(5m)C |
| 62 | ASO 2 | mA-po- |
| | | lnGpslnApslnTpslnApslnApsApsAps(5OH)CpsGps(5m)Cps(5m) |
| | | CpsGps(5m)CpslnApslnGpslnApscp(5m)C |
| 113 | SARS-CoV- | attaaaggtttataccttcccaggtaacaaaccaaccaactttcgatctcttgtagatctgttctct |
| | 2 genome | aaacgaactttaaaatctgtgtggctgtcactcggctgcatgcttagtgcactcacgcagtata |
| | (Genbank | attaataactaattactgtcgttgacaggacacgagtaactcgtctatcttctgcaggctgctta |
| | Accession | cggtttcgtccgtgttgcagccgatcatcagcacatctaggtttcgtccgggtgtgaccgaaa |
| | No. | ggtaagatggagagccttgtccctggtttcaacgagaaaacacacgtccaactcagtttgcct |
| | NC_045512.2) | gttttacaggttcgcgacgtgctcgtacgtggctttggagactccgtggaggaggtcttatca |
| | | gaggcacgtcaacatcttaaagatggcacttgtggcttagtagaagttgaaaaaggcgttttg |
| | | cctcaacttgaacagccctatgtgttcatcaaacgttcggatgctcgaactgcacctcatggtc |
| | | atgttatggttgagctggtagcagaactcgaaggcattcagtacggtcgtagtggtgagaca |
| | | cttggtgtccttgtccctcatgtgggcgaaataccagtggcttaccgcaaggttcttcttcgtaa |
| | | gaacggtaataaaggagctggtggccatagttacggcgccgatctaaagtcatttgacttag |
| | | gcgacgagcttggcactgatccttatgaagattttcaagaaaactggaacactaaacatagca |
| | | gtggtgttacccgtgaactcatgcgtgagcttaacggaggggcatacactcgctatgtcgata |
| | | acaacttctgtggccctgatggctaccctcttgagtgcattaaagaccttctagcacgtgctgg |
| | | taaagcttcatgcactttgtccgaacaactggactttattgacactaagaggggtgtatactgct |
| | | gccgtgaacatgagcatgaaattgcttggtacacggaacgttctgaaaagagctatgaattg |
| | | cagacaccttttgaaattaaattggcaaagaaatttgacaccttcaatggggaatgtccaaattt |
| | | tgtatttcccttaaattccataatcaagactattcaaccaagggttgaaaagaaaaagcttgatg |
| | | gctttatgggtagaattcgatctgtctatccagttgcgtcaccaaatgaatgcaaccaaatgtg |
| | | cctttcaactctcatgaagtgtgatcattgtggtgaaacttcatggcagacgggcgattttgtta |
| | | aagccacttgcgaattttgtggcactgagaatttgactaaagaaggtgccactacttgtggtta |
| | | cttaccccaaaatgctgttgttaaaatttattgtccagcatgtcacaattcagaagtaggacctg |
| | | agcatagtcttgccgaataccataatgaatctggcttgaaaaccattcttcgtaagggtggtcg |
| | | cactattgcctttggaggctgtgtgttctcttatgttggttgccataacaagtgtgcctattgggtt |
| | | ccacgtgctagcgctaacataggttgtaaccatacaggtgttgttggagaaggttccgaaggt |
| | | cttaatgacaaccttcttgaaatactccaaaaagagaaagtcaacatcaatattgttggtgactt |
| | | taaacttaatgaagagatcgccattattttggcatctttttctgcttccacaagtgcttttgtggaa |
| | | actgtgaaaggtttggattataaagcattcaaacaaattgttgaatcctgtggtaattttaaagtt |
| | | acaaaaggaaaagctaaaaaaggtgcctggaatattggtgaacagaaatcaatactgagtc |
| | | ctctttatgcatttgcatcagaggctgctcgtgttgtacgatcaattttctcccgcactcttgaaa |
| | | ctgctcaaaattctgtgcgtgttttacagaaggccgctataacaatactagatggaatttcaca |
| | | gtattcactgagactcattgatgctatgatgttcacatctgatttggctactaacaatctagttgta |
| | | atggcctacattacaggtggtgttgttcagttgacttcgcagtggctaactaacatctttggcac |
| | | tgtttatgaaaaactcaaacccgtccttgattggcttgaagagaagtttaaggaaggtgtagag |
| | | tttcttagagacggttgggaaattgttaaatttatctcaacctgtgcttgtgaaattgtcggtgga |
| | | caaattgtcacctgtgcaaaggaaattaaggagagtgttcagacattctttaagcttgtaaata |
| | | aatttttggctttgtgtgctgactctatcattattggtggagctaaacttaaagccttgaatttaggt |
| | | gaaacatttgtcacgcactcaaagggattgtacagaaagtgtgttaaatccagagaagaaac |
| | | tggcctactcatgcctctaaaagccccaaaagaaattatcttcttagagggagaaacacttcc |
| | | cacagaagtgttaacagaggaagttgtcttgaaaactggtgatttacaaccattagaacaacc |
| | | tactagtgaagctgttgaagctccattggttggtacaccagtttgtattaacgggcttatgttgct |
| | | cgaaatcaaagacacagaaaagtactgtgcccttgcacctaatatgatggtaacaaacaata |
| | | ccttcacactcaaaggcggtgcaccaacaaaggttacttttggtgatgacactgtgatagaag |
| | | tgcaaggttacaagagtgtgaatatcacttttgaacttgatgaaaggattgataaagtacttaat |
| | | gagaagtgctctgcctatacagttgaactcggtacagaagtaaatgagttcgcctgtgttgtg |
| | | gcagatgctgtcataaaaactttgcaaccagtatctgaattacttacaccactgggcattgattt |
| | | agatgagtggagtatggctacatactacttatttgatgagtctggtgagtttaaattggcttcac |
| | | atatgtattgttctttctaccctccagatgaggatgaagaagaaggtgattgtgaagaagaaga |
| | | gtttgagccatcaactcaatatgagtatggtactgaagatgattaccaaggtaaacctttggaa |
| | | tttggtgccacttctgctgctcttcaacctgaagaagagcaagaagaagattggttagatgatg |
| | | atagtcaacaaactgttggtcaacaagacggcagtgaggacaatcagacaactactattcaa |
| | | acaattgttgaggttcaacctcaattagagatggaacttacaccagttgttcagactattgaagt |
| | | gaatagttttagtggttatttaaaacttactgacaatgtatacattaaaaatgcagacattgtgga |
| | | agaagctaaaaaggtaaaaccaacagtggttgttaatgcagccaatgtttaccttaaacatgg |
| | | aggaggtgttgcaggagccttaaataaggctactaacaatgccatgcaagttgaatctgatg |
| | | attacatagctactaatggaccacttaaagtgggtggtagttgtgttttaagcggacacaatctt |
| | | gctaaacactgtcttcatgttgtcggcccaaatgttaacaaaggtgaagacattcaacttcttaa |
| | | gagtgcttatgaaaattttaatcagcacgaagttctacttgcaccattattatcagctggtattttt |
| | | ggtgctgaccctatacattctttaagagtttgtgtagatactgttcgcacaaatgtctacttagct |
| | | gtctttgataaaaatctctatgacaaacttgtttcaagctttttggaaatgaagagtgaaaagca |
| | | agttgaacaaaagatcgctgagattcctaaagaggaagttaagccatttataactgaaagtaa |
| | | accttcagttgaacagagaaaacaagatgataagaaaatcaaagcttgtgttgaagaagttac |
| | | aacaactctggaagaaactaagttcctcacagaaaacttgttactttatattgacattaatggca |
| | | atcttcatccagattctgccactcttgttagtgacattgacatcactttcttaaagaaagatgctc |
| | | catatatagtgggtgatgttgttcaagagggtgttttaactgctgtggttatacctactaaaaag |
| | | gctggtggcactactgaaatgctagcgaaagctttgagaaaagtgccaacagacaattatat |
| | | aaccacttacccgggtcagggtttaaatggttacactgtagaggaggcaaagacagtgctta |
| | | aaaagtgtaaaagtgccttttacattctaccatctattatctctaatgagaagcaagaaattcttg |
| | | gaactgtttcttggaatttgcgagaaatgcttgcacatgcagaagaaacacgcaaattaatgc |
| | | ctgtctgtgtggaaactaaagccatagtttcaactatacagcgtaaatataagggtattaaaata |
| | | caagagggtgtggttgattatggtgctagattttacttttacaccagtaaaacaactgtagcgtc |
| | | acttatcaacacacttaacgatctaaatgaaactcttgttacaatgccacttggctatgtaacac |
| | | atggcttaaatttggaagaagctgctcggtatatgagatctctcaaagtgccagctacagtttct |
| | | gtttcttcacctgatgctgttacagcgtataatggttatcttacttcttcttctaaaacacctgaag |
| | | aacattttattgaaaccatctcacttgctggttcctataaagattggtcctattctggacaatctac |
| | | acaactaggtatagaatttcttaagagaggtgataaaagtgtatattacactagtaatcctacca |
| | | cattccacctagatggtgaagttatcacctttgacaatcttaagacacttctttctttgagagaag |
| | | tgaggactattaaggtgtttacaacagtagacaacattaacctccacacgcaagttgtggaca |
| | | tgtcaatgacatatggacaacagtttggtccaacttatttggatggagctgatgttactaaaata |
| | | aaacctcataattcacatgaaggtaaaacattttatgttttacctaatgatgacactctacgtgtt |
| | | gaggcttttgagtactaccacacaactgatcctagttttctgggtaggtacatgtcagcattaaa |
| | | tcacactaaaaagtggaaatacccacaagttaatggtttaacttctattaaatgggcagataac |
| | | aactgttatcttgccactgcattgttaacactccaacaaatagagttgaagtttaatccacctgct |
| | | ctacaagatgcttattacagagcaagggctggtgaagctgctaacttttgtgcacttatcttagc |
| | | ctactgtaataagacagtaggtgagttaggtgatgttagagaaacaatgagttacttgtttcaa |
| | | catgccaatttagattcttgcaaaagagtcttgaacgtggtgtgtaaaacttgtggacaacagc |
| | | agacaacccttaagggtgtagaagctgttatgtacatgggcacactttcttatgaacaatttaa |
| | | gaaaggtgttcagataccttgtacgtgtggtaaacaagctacaaaatatctagtacaacagga |
| | | gtcaccttttgttatgatgtcagcaccacctgctcagtatgaacttaagcatggtacatttacttg |
| | | tgctagtgagtacactggtaattaccagtgtggtcactataaacatataacttctaaagaaactt |
| | | tgtattgcatagacggtgctttacttacaaagtcctcagaatacaaaggtcctattacggatgttt |
| | | tctacaaagaaaacagttacacaacaaccataaaaccagttacttataaattggatggtgttgtt |
| | | tgtacagaaattgaccctaagttggacaattattataagaaagacaattcttatttcacagagca |
| | | accaattgatcttgtaccaaaccaaccatatccaaacgcaagcttcgataattttaagtttgtat |
| | | gtgataatatcaaatttgctgatgatttaaaccagttaactggttataagaaacctgcttcaaga |
| | | gagcttaaagttacatttttccctgacttaaatggtgatgtggtggctattgattataaacactac |
| | | acaccctcttttaagaaaggagctaaattgttacataaacctattgtttggcatgttaacaatgca |
| | | actaataaagccacgtataaaccaaatacctggtgtatacgttgtctttggagcacaaaacca |
| | | gttgaaacatcaaattcgtttgatgtactgaagtcagaggacgcgcagggaatggataatctt |
| | | gcctgcgaagatctaaaaccagtctctgaagaagtagtggaaaatcctaccatacagaaag |
| | | acgttcttgagtgtaatgtgaaaactaccgaagttgtaggagacattatacttaaaccagcaaa |
| | | taatagtttaaaaattacagaagaggttggccacacagatctaatggctgcttatgtagacaat |
| | | tctagtcttactattaagaaacctaatgaattatctagagtattaggtttgaaaacccttgctactc |
| | | atggtttagctgctgttaatagtgtcccttgggatactatagctaattatgctaagccttttcttaa |
| | | caaagttgttagtacaactactaacatagttacacggtgtttaaaccgtgtttgtactaattatatg |
| | | ccttatttctttactttattgctacaattgtgtacttttactagaagtacaaattctagaattaaagca |
| | | tctatgccgactactatagcaaagaatactgttaagagtgtcggtaaattttgtctagaggcttc |
| | | atttaattatttgaagtcacctaatttttctaaactgataaatattataatttggtttttactattaagtg |
| | | tttgcctaggttctttaatctactcaaccgctgctttaggtgttttaatgtctaatttaggcatgcctt |
| | | cttactgtactggttacagagaaggctatttgaactctactaatgtcactattgcaacctactgta |
| | | ctggttctataccttgtagtgtttgtcttagtggtttagattctttagacacctatccttctttagaaa |
| | | ctatacaaattaccatttcatcttttaaatgggatttaactgcttttggcttagttgcagagtggtttt |
| | | tggcatatattcttttcactaggtttttctatgtacttggattggctgcaatcatgcaattgtttttca |
| | | gctattttgcagtacattttattagtaattcttggcttatgtggttaataattaatcttgtacaaatgg |
| | | ccccgatttcagctatggttagaatgtacatcttctttgcatcattttattatgtatggaaaagttat |
| | | gtgcatgttgtagacggttgtaattcatcaacttgtatgatgtgttacaaacgtaatagagcaac |
| | | aagagtcgaatgtacaactattgttaatggtgttagaaggtccttttatgtctatgctaatggag |
| | | gtaaaggcttttgcaaactacacaattggaattgtgttaattgtgatacattctgtgctggtagta |
| | | catttattagtgatgaagttgcgagagacttgtcactacagtttaaaagaccaataaatcctact |
| | | gaccagtcttcttacatcgttgatagtgttacagtgaagaatggttccatccatctttactttgata |
| | | aagctggtcaaaagacttatgaaagacattctctctctcattttgttaacttagacaacctgaga |
| | | gctaataacactaaaggttcattgcctattaatgttatagtttttgatggtaaatcaaaatgtgaa |
| | | gaatcatctgcaaaatcagcgtctgtttactacagtcagcttatgtgtcaacctatactgttacta |
| | | gatcaggcattagtgtctgatgttggtgatagtgcggaagttgcagttaaaatgtttgatgctta |
| | | cgttaatacgttttcatcaacttttaacgtaccaatggaaaaactcaaaacactagttgcaactg |
| | | cagaagctgaacttgcaaagaatgtgtccttagacaatgtcttatctacttttatttcagcagctc |
| | | ggcaagggtttgttgattcagatgtagaaactaaagatgttgttgaatgtcttaaattgtcacatc |
| | | aatctgacatagaagttactggcgatagttgtaataactatatgctcacctataacaaagttgaa |
| | | aacatgacaccccgtgaccttggtgcttgtattgactgtagtgcgcgtcatattaatgcgcagg |
| | | tagcaaaaagtcacaacattgctttgatatggaacgttaaagatttcatgtcattgtctgaacaa |
| | | ctacgaaaacaaatacgtagtgctgctaaaaagaataacttaccttttaagttgacatgtgcaa |
| | | ctactagacaagttgttaatgttgtaacaacaaagatagcacttaagggtggtaaaattgttaat |
| | | aattggttgaagcagttaattaaagttacacttgtgttcctttttgttgctgctattttctatttaataa |
| | | cacctgttcatgtcatgtctaaacatactgacttttcaagtgaaatcataggatacaaggctatt |
| | | gatggtggtgtcactcgtgacatagcatctacagatacttgttttgctaacaaacatgctgatttt |
| | | gacacatggtttagccagcgtggtggtagttatactaatgacaaagcttgcccattgattgctg |
| | | cagtcataacaagagaagtgggttttgtcgtgcctggtttgcctggcacgatattacgcacaa |
| | | ctaatggtgactttttgcatttcttacctagagtttttagtgcagttggtaacatctgttacacacca |
| | | tcaaaacttatagagtacactgactttgcaacatcagcttgtgttttggctgctgaatgtacaatt |
| | | tttaaagatgcttctggtaagccagtaccatattgttatgataccaatgtactagaaggttctgtt |
| | | gcttatgaaagtttacgccctgacacacgttatgtgctcatggatggctctattattcaatttcct |
| | | aacacctaccttgaaggttctgttagagtggtaacaacttttgattctgagtactgtaggcacg |
| | | gcacttgtgaaagatcagaagctggtgtttgtgtatctactagtggtagatgggtacttaacaat |
| | | gattattacagatctttaccaggagttttctgtggtgtagatgctgtaaatttacttactaatatgttt |
| | | acaccactaattcaacctattggtgctttggacatatcagcatctatagtagctggtggtattgta |
| | | gctatcgtagtaacatgccttgcctactattttatgaggtttagaagagcttttggtgaatacagt |
| | | catgtagttgcctttaatactttactattccttatgtcattcactgtactctgtttaacaccagtttact |
| | | cattcttacctggtgtttattctgttatttacttgtacttgacattttatcttactaatgatgtttctttttt |
| | | agcacatattcagtggatggttatgttcacacctttagtacctttctggataacaattgcttatatc |
| | | atttgtatttccacaaagcatttctattggttctttagtaattacctaaagagacgtgtagtctttaat |
| | | ggtgtttcctttagtacttttgaagaagctgcgctgtgcacctttttgttaaataaagaaatgtatc |
| | | taaagttgcgtagtgatgtgctattacctcttacgcaatataatagatacttagctctttataataa |
| | | gtacaagtattttagtggagcaatggatacaactagctacagagaagctgcttgttgtcatctc |
| | | gcaaaggctctcaatgacttcagtaactcaggttctgatgttctttaccaaccaccacaaacct |
| | | ctatcacctcagctgttttgcagagtggttttagaaaaatggcattcccatctggtaaagttgag |
| | | ggttgtatggtacaagtaacttgtggtacaactacacttaacggtctttggcttgatgacgtagt |
| | | ttactgtccaagacatgtgatctgcacctctgaagacatgcttaaccctaattatgaagatttact |
| | | cattcgtaagtctaatcataatttcttggtacaggctggtaatgttcaactcagggttattggaca |
| | | ttctatgcaaaattgtgtacttaagcttaaggttgatacagccaatcctaagacacctaagtata |
| | | agtttgttcgcattcaaccaggacagactttttcagtgttagcttgttacaatggttcaccatctg |
| | | gtgtttaccaatgtgctatgaggcccaatttcactattaagggttcattccttaatggttcatgtg |
| | | gtagtgttggttttaacatagattatgactgtgtctctttttgttacatgcaccatatggaattacca |
| | | actggagttcatgctggcacagacttagaaggtaacttttatggaccttttgttgacaggcaaa |
| | | cagcacaagcagctggtacggacacaactattacagttaatgttttagcttggttgtacgctgc |
| | | tgttataaatggagacaggtggtttctcaatcgatttaccacaactcttaatgactttaaccttgt |
| | | ggctatgaagtacaattatgaacctctaacacaagaccatgttgacatactaggacctctttct |
| | | gctcaaactggaattgccgttttagatatgtgtgcttcattaaaagaattactgcaaaatggtat |
| | | gaatggacgtaccatattgggtagtgctttattagaagatgaatttacaccttttgatgttgttag |
| | | acaatgctcaggtgttactttccaaagtgcagtgaaaagaacaatcaagggtacacaccact |
| | | ggttgttactcacaattttgacttcacttttagttttagtccagagtactcaatggtctttgttctttttt |
| | | ttgtatgaaaatgcctttttaccttttgctatgggtattattgctatgtctgcttttgcaatgatgtttg |
| | | tcaaacataagcatgcatttctctgtttgtttttgttaccttctcttgccactgtagcttattttaatat |
| | | ggtctatatgcctgctagttgggtgatgcgtattatgacatggttggatatggttgatactagttt |
| | | gtctggttttaagctaaaagactgtgttatgtatgcatcagctgtagtgttactaatccttatgaca |
| | | gcaagaactgtgtatgatgatggtgctaggagagtgtggacacttatgaatgtcttgacactc |
| | | gtttataaagtttattatggtaatgctttagatcaagccatttccatgtgggctcttataatctctgtt |
| | | acttctaactactcaggtgtagttacaactgtcatgtttttggccagaggtattgtttttatgtgtgt |
| | | tgagtattgccctattttcttcataactggtaatacacttcagtgtataatgctagtttattgtttctta |
| | | ggctatttttgtacttgttactttggcctcttttgtttactcaaccgctactttagactgactcttggt |
| | | gtttatgattacttagtttctacacaggagtttagatatatgaattcacagggactactcccaccc |
| | | aagaatagcatagatgccttcaaactcaacattaaattgttgggtgttggtggcaaaccttgtat |
| | | caaagtagccactgtacagtctaaaatgtcagatgtaaagtgcacatcagtagtcttactctca |
| | | gttttgcaacaactcagagtagaatcatcatctaaattgtgggctcaatgtgtccagttacacaa |
| | | tgacattctcttagctaaagatactactgaagcctttgaaaaaatggtttcactactttctgttttg |
| | | ctttccatgcagggtgctgtagacataaacaagctttgtgaagaaatgctggacaacagggc |
| | | aaccttacaagctatagcctcagagtttagttcccttccatcatatgcagcttttgctactgctca |
| | | agaagcttatgagcaggctgttgctaatggtgattctgaagttgttcttaaaaagttgaagaagt |
| | | ctttgaatgtggctaaatctgaatttgaccgtgatgcagccatgcaacgtaagttggaaaagat |
| | | ggctgatcaagctatgacccaaatgtataaacaggctagatctgaggacaagagggcaaaa |
| | | gttactagtgctatgcagacaatgcttttcactatgcttagaaagttggataatgatgcactcaa |
| | | caacattatcaacaatgcaagagatggttgtgttcccttgaacataatacctcttacaacagca |
| | | gccaaactaatggttgtcataccagactataacacatataaaaatacgtgtgatggtacaacat |
| | | ttacttatgcatcagcattgtgggaaatccaacaggttgtagatgcagatagtaaaattgttcaa |
| | | cttagtgaaattagtatggacaattcacctaatttagcatggcctcttattgtaacagctttaagg |
| | | gccaattctgctgtcaaattacagaataatgagcttagtcctgttgcactacgacagatgtcttg |
| | | tgctgccggtactacacaaactgcttgcactgatgacaatgcgttagcttactacaacacaac |
| | | aaagggaggtaggtttgtacttgcactgttatccgatttacaggatttgaaatgggctagattcc |
| | | ctaagagtgatggaactggtactatctatacagaactggaaccaccttgtaggtttgttacaga |
| | | cacacctaaaggtcctaaagtgaagtatttatactttattaaaggattaaacaacctaaatagag |
| | | gtatggtacttggtagtttagctgccacagtacgtctacaagctggtaatgcaacagaagtgc |
| | | ctgccaattcaactgtattatctttctgtgcttttgctgtagatgctgctaaagcttacaaagattat |
| | | ctagctagtgggggacaaccaatcactaattgtgttaagatgttgtgtacacacactggtactg |
| | | gtcaggcaataacagttacaccggaagccaatatggatcaagaatcctttggtggtgcatcgt |
| | | gttgtctgtactgccgttgccacatagatcatccaaatcctaaaggattttgtgacttaaaaggt |
| | | aagtatgtacaaatacctacaacttgtgctaatgaccctgtgggttttacacttaaaaacacagt |
| | | ctgtaccgtctgcggtatgtggaaaggttatggctgtagttgtgatcaactccgcgaacccat |
| | | gcttcagtcagctgatgcacaatcgtttttaaacgggtttgcggtgtaagtgcagcccgtctta |
| | | caccgtgcggcacaggcactagtactgatgtcgtatacagggcttttgacatctacaatgata |
| | | aagtagctggttttgctaaattcctaaaaactaattgttgtcgcttccaagaaaaggacgaaga |
| | | tgacaatttaattgattcttactttgtagttaagagacacactttctctaactaccaacatgaagaa |
| | | acaatttataatttacttaaggattgtccagctgttgctaaacatgacttctttaagtttagaatag |
| | | acggtgacatggtaccacatatatcacgtcaacgtcttactaaatacacaatggcagacctcg |
| | | tctatgctttaaggcattttgatgaaggtaattgtgacacattaaaagaaatacttgtcacataca |
| | | attgttgtgatgatgattatttcaataaaaaggactggtatgattttgtagaaaacccagatatatt |
| | | acgcgtatacgccaacttaggtgaacgtgtacgccaagctttgttaaaaacagtacaattctgt |
| | | gatgccatgcgaaatgctggtattgttggtgtactgacattagataatcaagatctcaatggta |
| | | actggtatgatttcggtgatttcatacaaaccacgccaggtagtggagttcctgttgtagattctt |
| | | attattcattgttaatgcctatattaaccttgaccagggctttaactgcagagtcacatgttgaca |
| | | ctgacttaacaaagccttacattaagtgggatttgttaaaatatgacttcacggaagagaggtt |
| | | aaaactctttgaccgttattttaaatattgggatcagacataccacccaaattgtgttaactgtttg |
| | | gatgacagatgcattctgcattgtgcaaactttaatgttttattctctacagtgttcccacctacaa |
| | | gttttggaccactagtgagaaaaatatttgttgatggtgttccatttgtagtttcaactggatacc |
| | | acttcagagagctaggtgttgtacataatcaggatgtaaacttacatagctctagacttagtttta |
| | | aggaattacttgtgtatgctgctgaccctgctatgcacgctgcttctggtaatctattactagata |
| | | aacgcactacgtgcttttcagtagctgcacttactaacaatgttgcttttcaaactgtcaaaccc |
| | | ggtaattttaacaaagacttctatgactttgctgtgtctaagggtttctttaaggaaggaagttct |
| | | gttgaattaaaacacttcttctttgctcaggatggtaatgctgctatcagcgattatgactactat |
| | | cgttataatctaccaacaatgtgtgatatcagacaactactatttgtagttgaagttgttgataag |
| | | tactttgattgttacgatggtggctgtattaatgctaaccaagtcatcgtcaacaacctagacaa |
| | | atcagctggttttccatttaataaatggggtaaggctagactttattatgattcaatgagttatga |
| | | ggatcaagatgcacttttcgcatatacaaaacgtaatgtcatccctactataactcaaatgaatc |
| | | ttaagtatgccattagtgcaaagaatagagctcgcaccgtagctggtgtctctatctgtagtact |
| | | atgaccaatagacagtttcatcaaaaattattgaaatcaatagccgccactagaggagctact |
| | | gtagtaattggaacaagcaaattctatggtggttggcacaacatgttaaaaactgtttatagtga |
| | | tgtagaaaaccctcaccttatgggttgggattatcctaaatgtgatagagccatgcctaacatg |
| | | cttagaattatggcctcacttgttcttgctcgcaaacatacaacgtgttgtagcttgtcacaccgt |
| | | ttctatagattagctaatgagtgtgctcaagtattgagtgaaatggtcatgtgtggcggttcact |
| | | atatgttaaaccaggtggaacctcatcaggagatgccacaactgcttatgctaatagtgttttta |
| | | acatttgtcaagctgtcacggccaatgttaatgcacttttatctactgatggtaacaaaattgcc |
| | | gataagtatgtccgcaatttacaacacagactttatgagtgtctctatagaaatagagatgttga |
| | | cacagactttgtgaatgagttttacgcatatttgcgtaaacatttctcaatgatgatactctctgac |
| | | gatgctgttgtgtgtttcaatagcacttatgcatctcaaggtctagtggctagcataaagaacttt |
| | | aagtcagttctttattatcaaaacaatgtttttatgtctgaagcaaaatgttggactgagactgac |
| | | cttactaaaggacctcatgaattttgctctcaacatacaatgctagttaaacagggtgatgatta |
| | | tgtgtaccttccttacccagatccatcaagaatcctaggggccggctgttttgtagatgatatcg |
| | | taaaaacagatggtacacttatgattgaacggttcgtgtctttagctatagatgcttacccactta |
| | | ctaaacatcctaatcaggagtatgctgatgtctttcatttgtacttacaatacataagaaagctac |
| | | atgatgagttaacaggacacatgttagacatgtattctgttatgcttactaatgataacacttcaa |
| | | ggtattgggaacctgagttttatgaggctatgtacacaccgcatacagtcttacaggctgttgg |
| | | ggcttgtgttctttgcaattcacagacttcattaagatgtggtgcttgcatacgtagaccattctta |
| | | tgttgtaaatgctgttacgaccatgtcatatcaacatcacataaattagtcttgtctgttaatccgt |
| | | atgtttgcaatgctccaggttgtgatgtcacagatgtgactcaactttacttaggaggtatgagc |
| | | tattattgtaaatcacataaaccacccattagttttccattgtgtgctaatggacaagtttttggttt |
| | | atataaaaatacatgtgttggtagcgataatgttactgactttaatgcaattgcaacatgtgactg |
| | | gacaaatgctggtgattacattttagctaacacctgtactgaaagactcaagctttttgcagca |
| | | gaaacgctcaaagctactgaggagacatttaaactgtcttatggtattgctactgtacgtgaag |
| | | tgctgtctgacagagaattacatctttcatgggaagttggtaaacctagaccaccacttaaccg |
| | | aaattatgtctttactggttatcgtgtaactaaaaacagtaaagtacaaataggagagtacacct |
| | | ttgaaaaaggtgactatggtgatgctgttgtttaccgaggtacaacaacttacaaattaaatgtt |
| | | ggtgattattttgtgctgacatcacatacagtaatgccattaagtgcacctacactagtgccaca |
| | | agagcactatgttagaattactggcttatacccaacactcaatatctcagatgagttttctagca |
| | | atgttgcaaattatcaaaaggttggtatgcaaaagtattctacactccagggaccacctggtac |
| | | tggtaagagtcattttgctattggcctagctctctactacccttctgctcgcatagtgtatacagc |
| | | ttgctctcatgccgctgttgatgcactatgtgagaaggcattaaaatatttgcctatagataaat |
| | | gtagtagaattatacctgcacgtgctcgtgtagagtgttttgataaattcaaagtgaattcaaca |
| | | ttagaacagtatgtcttttgtactgtaaatgcattgcctgagacgacagcagatatagttgtcttt |
| | | gatgaaatttcaatggccacaaattatgatttgagtgttgtcaatgccagattacgtgctaagca |
| | | ctatgtgtacattggcgaccctgctcaattacctgcaccacgcacattgctaactaagggcac |
| | | actagaaccagaatatttcaattcagtgtgtagacttatgaaaactataggtccagacatgttcc |
| | | tcggaacttgtcggcgttgtcctgctgaaattgttgacactgtgagtgctttggtttatgataata |
| | | agcttaaagcacataaagacaaatcagctcaatgctttaaaatgttttataagggtgttatcacg |
| | | catgatgtttcatctgcaattaacaggccacaaataggcgtggtaagagaattccttacacgta |
| | | accctgcttggagaaaagctgtctttatttcaccttataattcacagaatgctgtagcctcaaag |
| | | attttgggactaccaactcaaactgttgattcatcacagggctcagaatatgactatgtcatatt |
| | | cactcaaaccactgaaacagctcactcttgtaatgtaaacagatttaatgttgctattaccagag |
| | | caaaagtaggcatactttgcataatgtctgatagagacctttatgacaagttgcaatttacaagt |
| | | cttgaaattccacgtaggaatgtggcaactttacaagctgaaaatgtaacaggactctttaaag |
| | | attgtagtaaggtaatcactgggttacatcctacacaggcacctacacacctcagtgttgaca |
| | | ctaaattcaaaactgaaggtttatgtgttgacatacctggcatacctaaggacatgacctatag |
| | | aagactcatctctatgatgggttttaaaatgaattatcaagttaatggttaccctaacatgtttatc |
| | | acccgcgaagaagctataagacatgtacgtgcatggattggcttcgatgtcgaggggtgtca |
| | | tgctactagagaagctgttggtaccaatttacctttacagctaggtttttctacaggtgttaacct |
| | | agttgctgtacctacaggttatgttgatacacctaataatacagatttttccagagttagtgctaa |
| | | accaccgcctggagatcaatttaaacacctcataccacttatgtacaaaggacttccttggaat |
| | | gtagtgcgtataaagattgtacaaatgttaagtgacacacttaaaaatctctctgacagagtcg |
| | | tatttgtcttatgggcacatggctttgagttgacatctatgaagtattttgtgaaaataggacctg |
| | | agcgcacctgttgtctatgtgatagacgtgccacatgcttttccactgcttcagacacttatgcc |
| | | tgttggcatcattctattggatttgattacgtctataatccgtttatgattgatgttcaacaatgggg |
| | | ttttacaggtaacctacaaagcaaccatgatctgtattgtcaagtccatggtaatgcacatgtag |
| | | ctagttgtgatgcaatcatgactaggtgtctagctgtccacgagtgctttgttaagcgtgttgac |
| | | tggactattgaatatcctataattggtgatgaactgaagattaatgcggcttgtagaaaggttca |
| | | acacatggttgttaaagctgcattattagcagacaaattcccagttcttcacgacattggtaacc |
| | | ctaaagctattaagtgtgtacctcaagctgatgtagaatggaagttctatgatgcacagccttgt |
| | | agtgacaaagcttataaaatagaagaattattctattcttatgccacacattctgacaaattcaca |
| | | gatggtgtatgcctattttggaattgcaatgtcgatagatatcctgctaattccattgtttgtagatt |
| | | tgacactagagtgctatctaaccttaacttgcctggttgtgatggtggcagtttgtatgtaaataa |
| | | acatgcattccacacaccagcttttgataaaagtgcttttgttaatttaaaacaattaccatttttct |
| | | attactctgacagtccatgtgagtctcatggaaaacaagtagtgtcagatatagattatgtacc |
| | | actaaagtctgctacgtgtataacacgttgcaatttaggtggtgctgtctgtagacatcatgcta |
| | | atgagtacagattgtatctcgatgcttataacatgatgatctcagctggctttagcttgtgggttt |
| | | acaaacaatttgatacttataacctctggaacacttttacaagacttcagagtttagaaaatgtg |
| | | gcttttaatgttgtaaataagggacactttgatggacaacagggtgaagtaccagtttctatcat |
| | | taataacactgtttacacaaaagttgatggtgttgatgtagaattgtttgaaaataaaacaacatt |
| | | acctgttaatgtagcatttgagctttgggctaagcgcaacattaaaccagtaccagaggtgaa |
| | | aatactcaataatttgggtgtggacattgctgctaatactgtgatctgggactacaaaagagat |
| | | gctccagcacatatatctactattggtgtttgttctatgactgacatagccaagaaaccaactga |
| | | aacgatttgtgcaccactcactgtcttttttgatggtagagttgatggtcaagtagacttatttag |
| | | aaatgcccgtaatggtgttcttattacagaaggtagtgttaaaggtttacaaccatctgtaggtc |
| | | ccaaacaagctagtcttaatggagtcacattaattggagaagccgtaaaaacacagttcaatt |
| | | attataagaaagttgatggtgttgtccaacaattacctgaaacttactttactcagagtagaaatt |
| | | tacaagaatttaaacccaggagtcaaatggaaattgatttcttagaattagctatggatgaattc |
| | | attgaacggtataaattagaaggctatgccttcgaacatatcgtttatggagattttagtcatagt |
| | | cagttaggtggtttacatctactgattggactagctaaacgttttaaggaatcaccttttgaatta |
| | | gaagattttattcctatggacagtacagttaaaaactatttcataacagatgcgcaaacaggttc |
| | | atctaagtgtgtgtgttctgttattgatttattacttgatgattttgttgaaataataaaatcccaaga |
| | | tttatctgtagtttctaaggttgtcaaagtgactattgactatacagaaatttcatttatgctttggtg |
| | | taaagatggccatgtagaaacattttacccaaaattacaatctagtcaagcgtggcaaccggg |
| | | tgttgctatgcctaatctttacaaaatgcaaagaatgctattagaaaagtgtgaccttcaaaatta |
| | | tggtgatagtgcaacattacctaaaggcataatgatgaatgtcgcaaaatatactcaactgtgt |
| | | caatatttaaacacattaacattagctgtaccctataatatgagagttatacattttggtgctggtt |
| | | ctgataaaggagttgcaccaggtacagctgttttaagacagtggttgcctacgggtacgctgc |
| | | ttgtcgattcagatcttaatgactttgtctctgatgcagattcaactttgattggtgattgtgcaact |
| | | gtacatacagctaataaatgggatctcattattagtgatatgtacgaccctaagactaaaaatgt |
| | | tacaaaagaaaatgactctaaagagggttttttcacttacatttgtgggtttatacaacaaaagc |
| | | tagctcttggaggttccgtggctataaagataacagaacattcttggaatgctgatctttataag |
| | | ctcatgggacacttcgcatggtggacagcctttgttactaatgtgaatgcgtcatcatctgaag |
| | | catttttaattggatgtaattatcttggcaaaccacgcgaacaaatagatggttatgtcatgcatg |
| | | caaattacatattttggaggaatacaaatccaattcagttgtcttcctattctttatttgacatgagt |
| | | aaatttccccttaaattaaggggtactgctgttatgtctttaaaagaaggtcaaatcaatgatatg |
| | | attttatctcttcttagtaaaggtagacttataattagagaaaacaacagagttgttatttctagtg |
| | | atgttcttgttaacaactaaacgaacaatgtttgtttttcttgttttattgccactagtctctagtcag |
| | | tgtgttaatcttacaaccagaactcaattaccccctgcatacactaattctttcacacgtggtgttt |
| | | attaccctgacaaagttttcagatcctcagttttacattcaactcaggacttgttcttacctttctttt |
| | | ccaatgttacttggttccatgctatacatgtctctgggaccaatggtactaagaggtttgataac |
| | | cctgtcctaccatttaatgatggtgtttattttgcttccactgagaagtctaacataataagaggc |
| | | tggatttttggtactactttagattcgaagacccagtccctacttattgttaataacgctactaatg |
| | | ttgttattaaagtctgtgaatttcaattttgtaatgatccatttttgggtgtttattaccacaaaaaca |
| | | acaaaagttggatggaaagtgagttcagagtttattctagtgcgaataattgcacttttgaatat |
| | | gtctctcagccttttcttatggaccttgaaggaaaacagggtaatttcaaaaatcttagggaattt |
| | | gtgtttaagaatattgatggttattttaaaatatattctaagcacacgcctattaatttagtgcgtga |
| | | tctccctcagggtttttcggctttagaaccattggtagatttgccaataggtattaacatcactag |
| | | gtttcaaactttacttgctttacatagaagttatttgactcctggtgattcttcttcaggttggacag |
| | | ctggtgctgcagcttattatgtgggttatcttcaacctaggacttttctattaaaatataatgaaaa |
| | | tggaaccattacagatgctgtagactgtgcacttgaccctctctcagaaacaaagtgtacgttg |
| | | aaatccttcactgtagaaaaaggaatctatcaaacttctaactttagagtccaaccaacagaat |
| | | ctattgttagatttcctaatattacaaacttgtgcccttttggtgaagtttttaacgccaccagattt |
| | | gcatctgtttatgcttggaacaggaagagaatcagcaactgtgttgctgattattctgtcctatat |
| | | aattccgcatcattttccacttttaagtgttatggagtgtctcctactaaattaaatgatctctgcttt |
| | | actaatgtctatgcagattcatttgtaattagaggtgatgaagtcagacaaatcgctccagggc |
| | | aaactggaaagattgctgattataattataaattaccagatgattttacaggctgcgttatagctt |
| | | ggaattctaacaatcttgattctaaggttggtggtaattataattacctgtatagattgtttaggaa |
| | | gtctaatctcaaaccttttgagagagatatttcaactgaaatctatcaggccggtagcacacctt |
| | | gtaatggtgttgaaggttttaattgttactttcctttacaatcatatggtttccaacccactaatggt |
| | | gttggttaccaaccatacagagtagtagtactttcttttgaacttctacatgcaccagcaactgtt |
| | | tgtggacctaaaaagtctactaatttggttaaaaacaaatgtgtcaatttcaacttcaatggttta |
| | | acaggcacaggtgttcttactgagtctaacaaaaagtttctgcctttccaacaatttggcagag |
| | | acattgctgacactactgatgctgtccgtgatccacagacacttgagattcttgacattacacc |
| | | atgttcttttggtggtgtcagtgttataacaccaggaacaaatacttctaaccaggttgctgttctt |
| | | tatcaggatgttaactgcacagaagtccctgttgctattcatgcagatcaacttactcctacttg |
| | | gcgtgtttattctacaggttctaatgtttttcaaacacgtgcaggctgtttaataggggctgaaca |
| | | tgtcaacaactcatatgagtgtgacatacccattggtgcaggtatatgcgctagttatcagact |
| | | cagactaattctcctcggcgggcacgtagtgtagctagtcaatccatcattgcctacactatgt |
| | | cacttggtgcagaaaattcagttgcttactctaataactctattgccatacccacaaattttactat |
| | | tagtgttaccacagaaattctaccagtgtctatgaccaagacatcagtagattgtacaatgtac |
| | | atttgtggtgattcaactgaatgcagcaatcttttgttgcaatatggcagtttttgtacacaattaa |
| | | accgtgctttaactggaatagctgttgaacaagacaaaaacacccaagaagtttttgcacaag |
| | | tcaaacaaatttacaaaacaccaccaattaaagattttggtggttttaatttttcacaaatattacc |
| | | agatccatcaaaaccaagcaagaggtcatttattgaagatctacttttcaacaaagtgacactt |
| | | gcagatgctggcttcatcaaacaatatggtgattgccttggtgatattgctgctagagacctca |
| | | tttgtgcacaaaagtttaacggccttactgttttgccacctttgctcacagatgaaatgattgctc |
| | | aatacacttctgcactgttagcgggtacaatcacttctggttggacctttggtgcaggtgctgc |
| | | attacaaataccatttgctatgcaaatggcttataggtttaatggtattggagttacacagaatgt |
| | | tctctatgagaaccaaaaattgattgccaaccaatttaatagtgctattggcaaaattcaagact |
| | | cactttcttccacagcaagtgcacttggaaaacttcaagatgtggtcaaccaaaatgcacaag |
| | | ctttaaacacgcttgttaaacaacttagctccaattttggtgcaatttcaagtgttttaaatgatatc |
| | | ctttcacgtcttgacaaagttgaggctgaagtgcaaattgataggttgatcacaggcagacttc |
| | | aaagtttgcagacatatgtgactcaacaattaattagagctgcagaaatcagagcttctgctaa |
| | | tcttgctgctactaaaatgtcagagtgtgtacttggacaatcaaaaagagttgatttttgtggaa |
| | | agggctatcatcttatgtccttccctcagtcagcacctcatggtgtagtcttcttgcatgtgactt |
| | | atgtccctgcacaagaaaagaacttcacaactgctcctgccatttgtcatgatggaaaagcac |
| | | actttcctcgtgaaggtgtctttgtttcaaatggcacacactggtttgtaacacaaaggaattttt |
| | | atgaaccacaaatcattactacagacaacacatttgtgtctggtaactgtgatgttgtaatagga |
| | | attgtcaacaacacagtttatgatcctttgcaacctgaattagactcattcaaggaggagttaga |
| | | taaatattttaagaatcatacatcaccagatgttgatttaggtgacatctctggcattaatgcttca |
| | | gttgtaaacattcaaaaagaaattgaccgcctcaatgaggttgccaagaatttaaatgaatctc |
| | | tcatcgatctccaagaacttggaaagtatgagcagtatataaaatggccatggtacatttggct |
| | | aggttttatagctggcttgattgccatagtaatggtgacaattatgctttgctgtatgaccagttg |
| | | ctgtagttgtctcaagggctgttgttcttgtggatcctgctgcaaatttgatgaagacgactctg |
| | | agccagtgctcaaaggagtcaaattacattacacataaacgaacttatggatttgtttatgaga |
| | | atcttcacaattggaactgtaactttgaagcaaggtgaaatcaaggatgctactccttcagattt |
| | | tgttcgcgctactgcaacgataccgatacaagcctcactccctttcggatggcttattgttggc |
| | | gttgcacttcttgctgtttttcagagcgcttccaaaatcataaccctcaaaaagagatggcaact |
| | | agcactctccaagggtgttcactttgtttgcaacttgctgttgttgtttgtaacagtttactcacac |
| | | cttttgctcgttgctgctggccttgaagccccttttctctatctttatgctttagtctacttcttgcag |
| | | agtataaactttgtaagaataataatgaggctttggctttgctggaaatgccgttccaaaaacc |
| | | cattactttatgatgccaactattttctttgctggcatactaattgttacgactattgtataccttaca |
| | | atagtgtaacttcttcaattgtcattacttcaggtgatggcacaacaagtcctatttctgaacatg |
| | | actaccagattggtggttatactgaaaaatgggaatctggagtaaaagactgtgttgtattaca |
| | | cagttacttcacttcagactattaccagctgtactcaactcaattgagtacagacactggtgttg |
| | | aacatgttaccttcttcatctacaataaaattgttgatgagcctgaagaacatgtccaaattcac |
| | | acaatcgacggttcatccggagttgttaatccagtaatggaaccaatttatgatgaaccgacg |
| | | acgactactagcgtgcctttgtaagcacaagctgatgagtacgaacttatgtactcattcgtttc |
| | | ggaagagacaggtacgttaatagttaatagcgtacttctttttcttgctttcgtggtattcttgcta |
| | | gttacactagccatccttactgcgcttcgattgtgtgcgtactgctgcaatattgttaacgtgagt |
| | | cttgtaaaaccttctttttacgtttactctcgtgttaaaaatctgaattcttctagagttcctgatcttc |
| | | tggtctaaacgaactaaatattatattagtttttctgtttggaactttaattttagccatggcagatt |
| | | ccaacggtactattaccgttgaagagcttaaaaagctccttgaacaatggaacctagtaatag |
| | | gtttcctattccttacatggatttgtcttctacaatttgcctatgccaacaggaataggtttttgtat |
| | | ataattaagttaattttcctctggctgttatggccagtaactttagcttgttttgtgcttgctgctgttt |
| | | acagaataaattggatcaccggtggaattgctatcgcaatggcttgtcttgtaggcttgatgtg |
| | | gctcagctacttcattgcttctttcagactgtttgcgcgtacgcgttccatgtggtcattcaatcc |
| | | agaaactaacattcttctcaacgtgccactccatggcactattctgaccagaccgcttctagaa |
| | | agtgaactcgtaatcggagctgtgatccttcgtggacatcttcgtattgctggacaccatctag |
| | | gacgctgtgacatcaaggacctgcctaaagaaatcactgttgctacatcacgaacgctttctt |
| | | attacaaattgggagcttcgcagcgtgtagcaggtgactcaggttttgctgcatacagtcgct |
| | | acaggattggcaactataaattaaacacagaccattccagtagcagtgacaatattgctttgct |
| | | tgtacagtaagtgacaacagatgtttcatctcgttgactttcaggttactatagcagagatattac |
| | | taattattatgaggacttttaaagtttccatttggaatcttgattacatcataaacctcataattaaa |
| | | aatttatctaagtcactaactgagaataaatattctcaattagatgaagagcaaccaatggaga |
| | | ttgattaaacgaacatgaaaattattcttttcttggcactgataacactcgctacttgtgagcttta |
| | | tcactaccaagagtgtgttagaggtacaacagtacttttaaaagaaccttgctcttctggaacat |
| | | acgagggcaattcaccatttcatcctctagctgataacaaatttgcactgacttgctttagcact |
| | | caatttgcttttgcttgtcctgacggcgtaaaacacgtctatcagttacgtgccagatcagtttca |
| | | cctaaactgttcatcagacaagaggaagttcaagaactttactctccaatttttcttattgttgcg |
| | | gcaatagtgtttataacactttgcttcacactcaaaagaaagacagaatgattgaactttcatta |
| | | attgacttctatttgtgctttttagcctttctgctattccttgttttaattatgcttattatcttttggttctc |
| | | acttgaactgcaagatcataatgaaacttgtcacgcctaaacgaacatgaaatttcttgttttctt |
| | | aggaatcatcacaactgtagctgcatttcaccaagaatgtagtttacagtcatgtactcaacat |
| | | caaccatatgtagttgatgacccgtgtcctattcacttctattctaaatggtatattagagtagga |
| | | gctagaaaatcagcacctttaattgaattgtgcgtggatgaggctggttctaaatcacccattc |
| | | agtacatcgatatcggtaattatacagtttcctgtttaccttttacaattaattgccaggaacctaa |
| | | attgggtagtcttgtagtgcgttgttcgttctatgaagactttttagagtatcatgacgttcgtgtt |
| | | gttttagatttcatctaaacgaacaaactaaaatgtctgataatggaccccaaaatcagcgaaa |
| | | tgcaccccgcattacgtttggtggaccctcagattcaactggcagtaaccagaatggagaac |
| | | gcagtggggcgcgatcaaaacaacgtcggccccaaggtttacccaataatactgcgtcttg |
| | | gttcaccgctctcactcaacatggcaaggaagaccttaaattccctcgaggacaaggcgttc |
| | | caattaacaccaatagcagtccagatgaccaaattggctactaccgaagagctaccagacg |
| | | aattcgtggtggtgacggtaaaatgaaagatctcagtccaagatggtatttctactacctagga |
| | | actgggccagaagctggacttccctatggtgctaacaaagacggcatcatatgggttgcaac |
| | | tgagggagccttgaatacaccaaaagatcacattggcacccgcaatcctgctaacaatgctg |
| | | caatcgtgctacaacttcctcaaggaacaacattgccaaaaggcttctacgcagaagggag |
| | | cagaggcggcagtcaagcctcttctcgttcctcatcacgtagtcgcaacagttcaagaaattc |
| | | aactccaggcagcagtaggggaacttctcctgctagaatggctggcaatggcggtgatgct |
| | | gctcttgctttgctgctgcttgacagattgaaccagcttgagagcaaaatgtctggtaaaggcc |
| | | aacaacaacaaggccaaactgtcactaagaaatctgctgctgaggcttctaagaagcctcg |
| | | gcaaaaacgtactgccactaaagcatacaatgtaacacaagctttcggcagacgtggtcca |
| | | gaacaaacccaaggaaattttggggaccaggaactaatcagacaaggaactgattacaaac |
| | | attggccgcaaattgcacaatttgcccccagcgcttcagcgttcttcggaatgtcgcgcattg |
| | | gcatggaagtcacaccttcgggaacgtggttgacctacacaggtgccatcaaattggatgac |
| | | aaagatccaaatttcaaagatcaagtcattttgctgaataagcatattgacgcatacaaaacatt |
| | | cccaccaacagagcctaaaaaggacaaaaagaagaaggctgatgaaactcaagccttacc |
| | | gcagagacagaagaaacagcaaactgtgactcttcttcctgctgcagatttggatgatttctcc |
| | | aaacaattgcaacaatccatgagcagtgctgactcaactcaggcctaaactcatgcagacca |
| | | cacaaggcagatgggctatataaacgttttcgcttttccgtttacgatatatagtctactcttgtg |
| | | cagaatgaattctcgtaactacatagcacaagtagatgtagttaactttaatctcacatagcaat |
| | | ctttaatcagtgtgtaacattagggaggacttgaaagagccaccacattttcaccgaggccac |
| | | gcggagtacgatcgagtgtacagtgaacaatgctagggagagctgcctatatggaagagc |
| | | cctaatgtgtaaaattaattttagtagtgctatccccatgtgattttaatagcttcttaggagaatg |
| | | acaaaaaaaaaaaaaaaaaaaaaaaaaaaaaaaaa |

⁺ln = Locked nucleic acid (LNA) = 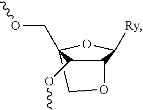
wherein Ry is a nucleobase;
lnA = Locked nucleic acid (LNA) A;
ln(5m)C = ln(5m)C = Locked nucleic acid (LNA)-5 methyl C;
lnG = Locked nucleic acid (LNA) G;
lnT = Locked nucleic acid (LNA) T;
(5m)C = 5 methyl C;
scp = = spirocyclopropyl;
scp(5m)C = cyclopropyl-5 methyl C;
(5OH)C = 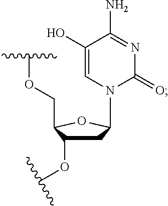
po = phosphodiester linkage;
ps = phosphorothioate linkage

What is claimed is:

1. A nucleotide phosphate mimic comprising a structure of: 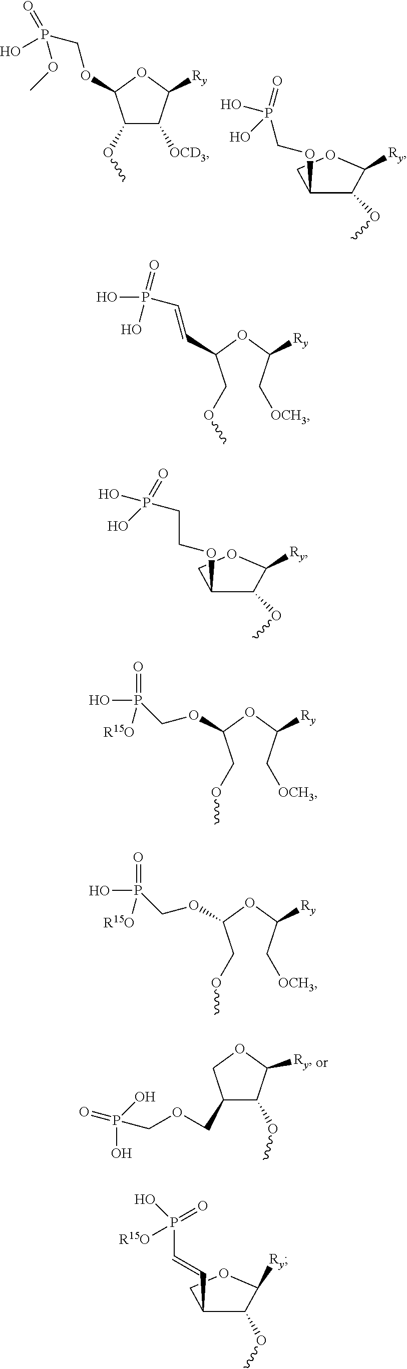
wherein R_{y} is a nucleobase and R¹⁵ is H or CH₃, and wherein a represents a phosphodiester linkage.

2. The nucleotide phosphate mimic of claim 1, wherein the nucleotide phosphate mimic comprises a structure of:

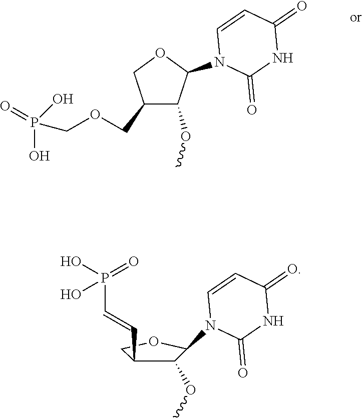

3. A short interfering nucleic acid (siNA), comprising a sense strand and an antisense strand, and comprising at least one nucleotide phosphate mimic selected from: 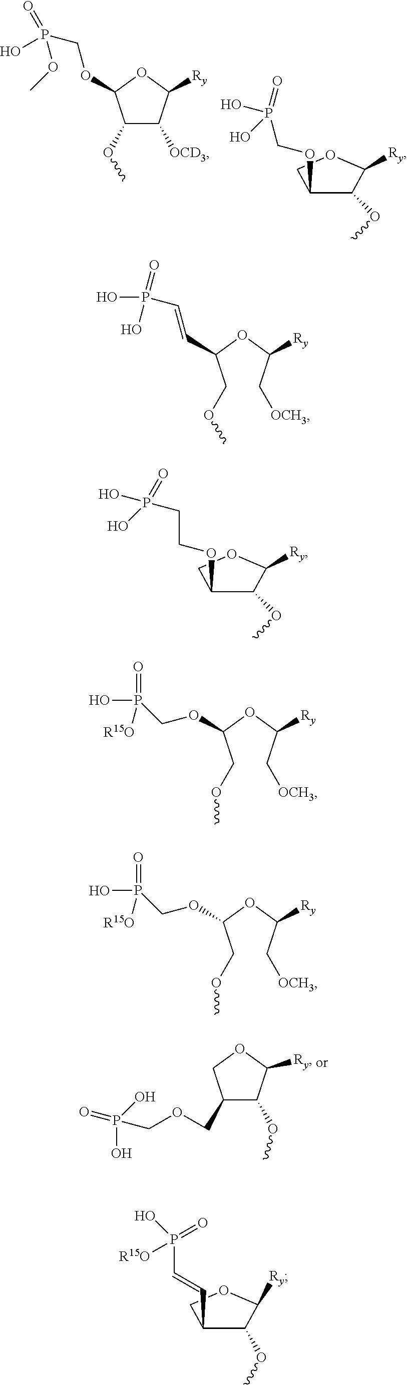
wherein R_{y} is a nucleobase and R¹⁵ is H or CH₃, and wherein ξ represents a phosphodiester linkage.

4. The siNA of claim 3, wherein the at least one nucleotide phosphate mimic is located at the 5′ end of the sense strand.

5. The siNA of claim 3, wherein the at least one nucleotide phosphate mimic is located at the 3′ end of the sense strand.

6. The siNA of claim 3, wherein the at least one nucleotide phosphate mimic is located at the 5′ end of the antisense strand.

7. The siNA of claim 3, wherein the at least one nucleotide phosphate mimic is located at the 3′ end of the antisense strand.

8. A short interfering nucleic acid (siNA) comprising:
(a) a sense strand comprising a first nucleotide sequence that is at least about 90% identical to an RNA corresponding to a target gene, wherein the first nucleotide sequence:
(i) is 15 to 30 nucleotides in length; and
(ii) comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 3, 5, 7, 8, 9, 10, 11, 12, 14, 17, and/or 19 from the 5′ end of the first nucleotide sequence is a 2′-fluoro nucleotide or wherein at least one modified nucleotide is a 2′-O-methyl nucleotide and at least one modified nucleotide is a 2′-fluoro nucleotide; and
an antisense strand comprising a second nucleotide sequence that is at least about 90% complementary to the RNA corresponding to the target gene, wherein the second nucleotide sequence:
(iii) is 15 to 30 nucleotides in length; and
(iv) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; or
(b) a sense strand comprising a first nucleotide sequence that is at least about 90% identical to an RNA corresponding to a target gene, wherein the first nucleotide sequence:
(i) is 15 to 30 nucleotides in length; and
(ii) comprises 15 or more modified nucleotides independently selected from a 2′-O-methyl nucleotide and a 2′-fluoro nucleotide, wherein at least one modified nucleotide is a 2′-O-methyl nucleotide and at least one modified nucleotide is a 2′-fluoro nucleotide; and
an antisense strand comprising a second nucleotide sequence that is at least about 90% complementary to the RNA corresponding to the target gene, wherein the second nucleotide sequence:
(iii) is 15 to 30 nucleotides in length; and
(iv) comprises 15 or more modified nucleotides independently selected from a 2′-O-methyl nucleotide and a 2′-fluoro nucleotide, wherein at least one modified nucleotide is a 2′-O-methyl nucleotide and the nucleotide at position 2, 5, 6, 8, 10, 14, 16, 17, and/or 18 from the 5′ end of the second nucleotide sequence is a 2′-fluoro nucleotide;
wherein the sense strand and/or the antisense strand comprise at least one nucleotide phosphate mimic according to claim 1.

9. The siNA of claim 8, wherein the antisense strand comprises the at least one nucleotide phosphate mimic at its 5′ end.

10. The siNA of claim 8, wherein the sense strand and/or the antisense strand independently comprise 1 or more phosphorothioate internucleoside linkages, 1 or more mesyl phosphoroamidate internucleoside linkages, or a combination thereof.

11. The siNA molecule of claim 10, wherein:
(i) at least one phosphorothioate internucleoside linkage in the sense strand is between the nucleotides at positions 1 and 2 from the 5′ end of the first nucleotide sequence;
(ii) at least one phosphorothioate internucleoside linkage is between the nucleotides at positions 2 and 3 from the 5′ end of the first nucleotide sequence;
(ii) at least one phosphorothioate internucleoside linkage in the antisense strand is between the nucleotides at positions 1 and 2 from the 5′ end of the second nucleotide sequence;
(iii) at least one phosphorothioate internucleoside linkage in the antisense strand is between the nucleotides at positions 2 and 3 from the 5′ end of the second nucleotide sequence;
(iv) at least one phosphorothioate internucleoside linkage in the antisense strand is between the nucleotides at positions 1 and 2 from the 3′ end of the second nucleotide sequence;
(v) at least one phosphorothioate internucleoside linkage is between the nucleotides at positions 2 and 3 from the 3′ end of the second nucleotide sequence;
(vi) at least one mesyl phosphoroamidate internucleoside linkage in the sense strand is between the nucleotides at positions 1 and 2 from the 5′ end of the first nucleotide sequence;
(vii) at least one mesyl phosphoroamidate internucleoside linkage is between the nucleotides at positions 2 and 3 from the 5′ end of the first nucleotide sequence;
(viii) at least one mesyl phosphoroamidate internucleoside linkage in the antisense strand is between the nucleotides at positions 1 and 2 from the 5′ end of the second nucleotide sequence;
(ix) at least one mesyl phosphoroamidate internucleoside linkage in the antisense strand is between the nucleotides at positions 2 and 3 from the 5′ end of the second nucleotide sequence;
(x) at least one mesyl phosphoroamidate internucleoside linkage in the antisense strand is between the nucleotides at positions 1 and 2 from the 3′ end of the second nucleotide sequence; and/or
(xi) at least one mesyl phosphoroamidate internucleoside linkage is between the nucleotides at positions 2 and 3 from the 3′ end of the second nucleotide sequence.

12. The siNA of claim 8, wherein the siNA further comprises a phosphorylation blocker.

13. The siNA of claim 8, wherein the siNA further comprises a galactosamine.

14. The siNA of claim 13, wherein the galactosamine is N-acetylgalactosamine (GalNAc) of Formula (VI): 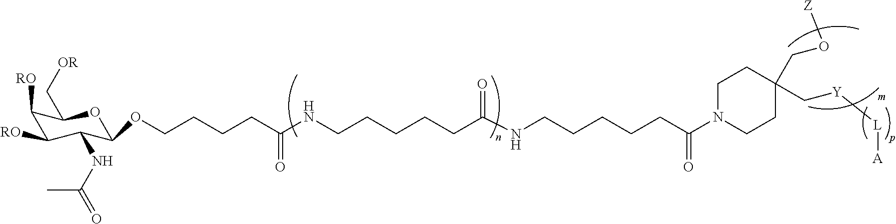
wherein
m is 1, 2, 3, 4, or 5;
each n is independently 1 or 2; p is 0 or 1;
each R is independently H;
each Y is independently selected from —O—P(═O)(SH)—, —O—P(═O)(O)—, —O—P(═O)(OH)—, and —O—P(S)S—;
Z is H or a second protecting group;
either L is a linker or L and Y in combination are a linker; and
A is H, OH, a third protecting group, an activated group, or an oligonucleotide.

15. The siNA of claim 14, wherein the galactosamine is N-acetylgalactosamine (GalNAc) of Formula (VII):

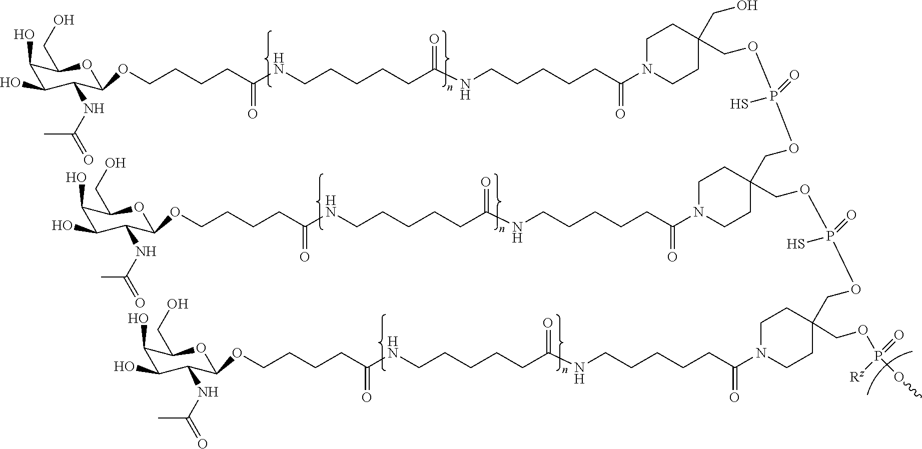

16. The siNA molecule of claim 8, wherein the target gene is a viral gene.

17. The siNA of claim 8, further comprising at least one nucleotide selected from 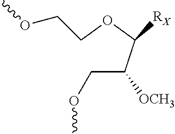
wherein Rₓ is a nucleobase, aryl, heteroaryl, or H; 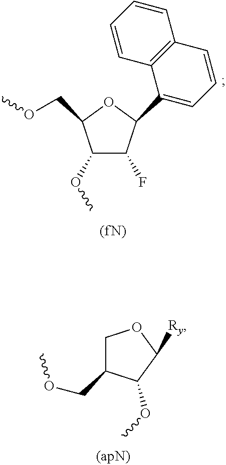
wherein R_{y} is a nucleobase; and any combination thereof.

18. A pharmaceutical composition, comprising the siNA according to claim 8 and a pharmaceutically acceptable carrier.

19. A method of treating a disease in a subject in need thereof, comprising administering to the subject the siNA according to claim 8.

20. A short interfering nucleic acid (siNA) comprising a sense strand and an antisense strand, wherein the sense strand and/or the antisense strand independently comprise at least 1 mesyl phosphoroamidate internucleoside linkages.
